(19) United States
(12) Patent Application Publication
(10) Pub. No.: US 2026/0077130 A1
(43) Pub. Date: Mar. 19, 2026

(54) SYSTEMS AND METHODS FOR PRECISION DOSE DELIVERY, STERLIZATION, AND PACKAGING OF MEDICAL DEVICES

(71) Applicant: Regeneron Pharmaceuticals, Inc., Tarrytown, NY (US)

(72) Inventors: Kenneth F. LORENZE, Red Hook, NY (US); Dustin BLODGETT, Altamont, NY (US); Trevor LANGLEY, Rensselaer, NY (US)

(73) Assignee: Regeneron Pharmaceuticals, Inc., Tarrytown, NY (US)

(21) Appl. No.: 19/331,614

(22) Filed: Sep. 17, 2025

Related U.S. Application Data
(60) Provisional application No. 63/696,350, filed on Sep. 18, 2024.
(60) Provisional application No. 63/796,227, filed on Apr. 28, 2025.

(51) Int. Cl. A61M 5/31; A61K 9/00; A61K 38/17; A61L 2/00; A61L 2/20; A61L 101/02; A61M 5/315

(52) U.S. Cl. CPC A61M 5/3146; A61K 9/0019; A61K 38/179; A61M 5/31505; A61M 5/31513; A61L 2/20; A61L 2/208; A61L 2101/02; A61L 2103/05; A61L 2103/23; A61M 2210/0612

(57) ABSTRACT

A drug delivery device that includes a body storing a VEGF antagonist, a plunger rod contacting a stopper inside the body, and a flange coupled to the body. The body is silicone-free. The plunger rod includes protrusions and the flange includes a proximal collar and slots positioned along the proximal collar. The flange is configured such that when the protrusions and slots are misaligned, distal movement of the plunger rod relative to the body is limited upon the protrusions contacting the proximal collar to prime the drug delivery device. The flange is configured such that when the protrusions and slots are aligned, distal movement of the plunger rod relative to the body is limited upon the protrusions extending into the slots to deliver the VEGF antagonist.

### CROSS-REFERENCE TO RELATED APPLICATIONS

This application claims priority to U.S. Provisional Application No. 63/696,350, filed Sep. 18, 2024, and U.S. Provisional Application No. 63/796,227, filed Apr. 28, 2025, each of which is incorporated by reference herein in their entireties.

### FIELD OF DISCLOSURE

Aspects of the present disclosure relate to devices, systems, and methods for priming or otherwise configuring a dose delivery device, e.g., a syringe, to promote precision dose delivery, along with systems and methods for sterilizing and/or packaging such dose delivery devices. More specifically, embodiments of the present disclosure relate to devices and methods for loading, storing, transporting, and/or delivering precise doses of a drug substance or other fluid substance. Some embodiments of the present disclosure relate to systems and methods for chemical sterilization (e.g., moist chemical sterilization), including terminal sterilization of dose delivery devices using vaporized sterilant, such as vaporized hydrogen peroxide. Additionally, embodiments of the present disclosure relate to a blister packaging for housing the dose delivery device.

Various embodiments of the present disclosure further relate to sterilization systems and methods for sterilization. More specifically, some embodiments of the present disclosure relate to systems and methods for chemical sterilization (e.g., moist chemical sterilization) of medical products, including terminal sterilization of drug delivery devices (e.g., pre-filled and packaged syringes) using vaporized sterilant. Additionally, embodiments of the present disclosure relate to systems and methods for monitoring and controlling environments and/or conditions within a sterilization apparatus or process.

### INTRODUCTION

### Dose Delivery Device and Medicament

Drug products including fluid drug substances may be deliverable to patients in a variety of ways, including via injection. In many cases, the precision and accuracy of a liquid drug product's volume is crucial. For example, medical professionals may have an interest in ensuring that an approved or prescribed volume of a drug substance is consistently delivered to each patient requiring the drug. Additionally, over- or under-dosing a patient with a drug substance, even slightly, may have an undesired (or even negative) clinical impact on the patient. Moreover, some drug products are prescribed at low volumes (e.g., under 100 μL). At low volumes, human error in preparing and delivering an accurate dose of a drug substance for injection may impact the drug's efficacy in a patient and the subsequent clinical effect on the patient.

Additional aspects of fluid drug delivery can complicate the goal of accurate dose delivery via injection. For example, for a correct dose of a drug substance to be dispensed from a device (e.g., a syringe), a corresponding accurate volume of the substance must be loaded into the device. Furthermore, handling, storage, packaging, and/or transportation of loaded devices must not result in inadvertent expulsion of drug substance from the devices. Additionally, prior to administration of a drug substance from a device, the device may need to be primed, e.g., to remove air bubbles and excess drug substance from within the device's needle and barrel. Incorrectly priming a device may result in expulsion of too much or too little drug substance from the device, which likewise may result in a decreased dose being delivered to a patient, or air bubbles being injected from the device into the patient.

The present disclosure also addresses needs unmet by prior publications WO 2018/232408, published on Dec. 20, 2018; WO 2018/224640, published on Dec. 13, 2018; and WO 2018/224644, published on Dec. 13, 2018. Further, at least some embodiments of the present disclosure include features that are different than those features disclosed in publication WO 2019/118588, published on Jun. 20, 2019. Some features include, for example, a plunger rod including one or more extensions extending distally from an actuation portion of the plunger rod and having hook or clip shaped parts for receipt with side openings of a flange piece. The plunger rod may include a neck having three or more sections, each having a different cross-sectional profile and/or shape relative to one another. Further features may include, for example, a flange piece including a collar having one or more internal grooves for receiving the hook or clip shaped parts of the one or more extensions, thereby allowing the extensions to flex radially-outward from a compressed configuration to an expanded configuration. The flange piece may include an opening configured to receive each of the three or more sections of the neck based on, for example, a rotational arrangement of the plunger rod relative to the flange piece.

A flange piece of the present disclosure may further include one or more movable ribs and/or one or more movable tabs for engaging a syringe body to couple the flange piece to the syringe body. For example, one or more movable ribs may be positioned proximal to a lip and lateral opening of the flange piece for receiving a top flange of the syringe body. The movable ribs may move, deflect, and/or deform in response to receiving the top flange through the lateral opening, and may be configured to apply a distally-directed force onto the top flange to secure the syringe body to the flange piece. By way of further example, the one or more movable tabs may be positioned distal to the lip and lateral opening. The movable tabs may move, deflect, and/or deform in response to the flange piece receiving the syringe body, and may be configured to apply a radially-directed force onto the syringe body to secure the syringe body to the flange piece. It should be appreciated that embodiments of the present disclosure include various other features shown and described herein that are different than those features disclosed in publication WO2019/118588.

### Methods of Treatment & Administration of Medicament

Neovascular (wet) AMD (nAMD) is a major health issue in aging populations globally. Vision loss in nAMD results from the abnormal growth and leakage of blood vessels in the macula. In elderly patients affected by nAMD, vision loss frequently has an even greater impact, as it substantially reduces the visual compensation of functional impairment by other age-related comorbidities, such as arthritis and osteoporosis. Diabetic retinopathy (DR) is the most common microvascular complication of diabetes. Diabetic macular edema (DME), a manifestation of diabetic retinopathy, is the primary cause of vision loss and blindness in subjects with diabetes and the most frequent cause of blindness in young and middle-aged adults. If left untreated, approximately half of subjects with DME will lose 2 or more lines of visual acuity (VA) within 2 years. Among subjects with diabetes, the prevalence of clinically significant macular edema (CSME) ranges from 2.77% to 7.6%.

Intravitreally (IVT) administered anti-vascular endothelial growth factor (VEGF) therapies like EYLEA® inhibit neovascular vessel growth and leakage in the retina, and they are currently the standard-of-care for patients with nAMD or DME. They not only maintain visual function but also provide clinically meaningful visual gains. Treatment of nAMD and/or DME is chronic and life-long in most patients to suppress retinal edema and recurrences of choroidal neovascularization (CNV). Although the currently approved IVT anti-VEGF therapies are efficacious and well-tolerated, the need for IVT injections every 4 to 8 weeks, specifically in the initial phase and during maintenance of treatment, represents a significant burden to physicians, patients, and caregivers. While the procedure is straightforward and relatively easy to perform, capacity issues for ensuring an appropriate injection frequency in order to achieve patient outcomes similar to those seen in the pivotal studies represent an increasing challenge to individual practices and the healthcare system, overall. Moreover, high frequency dosing leads to increased burdens on patients, e.g., to find transportation and miss work. A secondary effect of this burden is a lower probability of non-compliance with the prescribed treatment regimen.

While the efficacy and safety of currently approved VEGF antagonist therapies have been established for the treatment of nAMD and/or DME, there remains an unmet medical need for the development of therapies with the potential to reduce treatment burden while providing at least similar or even improved visual outcomes over currently available standard-of-care.

Increasing the molar concentration of VEGF antagonist therapeutic protein in the dosing formulation is a potential way to bring further benefits to subjects with chorioretinal vascular diseases, including DME. A higher dose of aflibercept administered IVT has the potential to prolong the drug's therapeutic effects. The resulting extension of treatment intervals early after the initiation of treatment to every 12 weeks or 16 weeks would reduce the number of injections in the first treatment year. A potential decrease in injection-related treatment burden and safety events with fewer injections could be a significant contribution to subject care and healthcare services.

EYLEA (2 mg dose, administered at a concentration of 40 mg/mL, also called intravitreal aflibercept injection [IAI]) is currently approved in the United States (US) for the treatment of nAMD, and is also approved for the treatment of macular edema following retinal vein occlusion (RVO), diabetic macular edema (DME), and diabetic retinopathy (DR).

### Packaging of Dose Delivery Device

Many medical devices are packaged for distribution to healthcare facilities and/or for storage, for example. Once a healthcare provider is ready to use the medical device, the medical device is removed from the packaging and prepped, if necessary, for use on a patient. In order to remove medical devices from packaging, users often need to directly handle the devices and to push, pull, lift, slide, or otherwise directly touch the medical device with their hands. In some instances, it may not be desirable to handle the medical devices too much, e.g., for reasons of sterility or to inhibit damage to the medical devices, when removing the medical devices from their packaging.

For example, syringes, especially pre-filled syringes, may be contaminated when they are handled during removal of packaging, which may jeopardize the sterility of the syringe body, needle, needle attachment, and/or any medicament or other fluid contained within. If the pre-filled syringe is an ophthalmic syringe, handling during removal from packaging may lead to contamination of the syringe and/or medicament or other fluid contained within, which may in turn lead to infectious inflammation of the eye, e.g., intraocular infections, such as post-injection endophthalmitis.

Additionally, in the case of pre-filled syringes, handling of the syringe may jeopardize dose accuracy, e.g., if the syringe plunger is bumped during removal from the packaging. With ophthalmic syringes, dose accuracy may be more important, because the dose size may be relatively small, e.g., less than or equal to 20 ml, less than or equal to 15 ml, less than or equal to 10 ml, less than or equal to 0.5 ml, or less than or equal to 1 ml. For example, the dose may be less than or equal to 200 μl, less than or equal to 150 μl, or less than or equal to 100 μl. Accordingly, there exists a need for medical device packaging that decreases the amount of direct handling of the medical device, particularly pre-filled ophthalmic syringes, during removal from the packaging and/or provides easier removal of the medical device from the packaging.

### Systems & Methods of Sterilization

Chemical sterilization processes, such as processes using ethylene oxide, vaporized hydrogen peroxide, vaporized peracetic acid, and the like, offer many advantages, such as the ability to sterilize a load at relatively low temperatures (e.g., at less than 50° C.), and without the need to enter a deep vacuum (e.g., without decreasing a pressure to below 100 millibars). Such sterilization processes may be particularly useful in the sterilization of medical devices and medical products that are sensitive to extreme temperatures and/or pressures.

Processes using chemical sterilants include steps to ensure that the sterilant reaches all parts of a load that need to be sterilized, and that after sterilization has taken place, the sterilant is removed from the load to a degree that ensures the safety and efficacy of any products that were sterilized. Removing sterilant from the load may be referred to as aerating the load. Additionally, sterilization processes may benefit from improvements that reduce the time and resources required to sterilize and aerate/dry a load.

In particular, the use of vaporized chemicals, such as vaporized hydrogen peroxide, may raise certain challenges. The distribution of vaporized sterilant in a sterilization system, its behavior (e.g., condensation, evaporation, etc.), and its interaction with a sterilization load (e.g., adsorption to materials of the load), may affect its efficacy and how easily it may be removed from the load. Conventional sterilization processes may be less effective for some sterilization loads. For example, material and physical properties of a sterilization load may impact the distribution and movement of vaporized sterilant.

### SUMMARY

Disclosed herein are drug delivery devices, methods for treatment and administration using drug delivery devices, packaging for drug delivery devices, and systems and methods for sterilizing packaged drug delivery devices. In some aspects, the techniques described herein relate to a drug delivery device, including: a body storing a medicament, wherein the body is silicone-free; a plunger rod having a proximal portion, a distal portion contacting a stopper inside the body, and a pair of protrusions extending outwards from the proximal portion; and a flange coupled to the body, the flange including a proximal collar and a pair of slots positioned along the proximal collar; wherein the flange is configured such that when the pair of protrusions and the pair of slots are misaligned with one another, distal movement of the plunger rod relative to the body is limited upon the pair of protrusions contacting the proximal collar to prime the drug delivery device by expelling air or at least a portion of the medicament stored in the body; and wherein the flange is configured such that when the pair of protrusions and the pair of slots are aligned with one another, distal movement of the plunger rod relative to the body is limited upon the pair of protrusions extending into the pair of slots to deliver the medicament from the body.

In some aspects, the techniques described herein relate to a drug delivery device, wherein the plunger rod includes a pair of extensions extending distally from the proximal portion, and the flange includes a channel defined by an interior surface and a pair of grooves formed on the interior surface.

In some aspects, the techniques described herein relate to a drug delivery device, wherein the flange is configured such that the pair of extensions are misaligned with the pair of grooves when the pair of protrusions and the pair of slots are misaligned with one another.

In some aspects, the techniques described herein relate to a drug delivery device, wherein the interior surface is configured to engage the pair of extensions when the pair of protrusions and the pair of slots are misaligned and the plunger rod translates distally through the channel via a first stroke; and wherein the pair of grooves is configured to receive the pair of extensions when the pair of protrusions and the pair of slots are aligned and the plunger rod translates distally through the channel via a second stroke.

In some aspects, the techniques described herein relate to a drug delivery device, wherein the flange is configured to compress the pair of extensions radially inwards relative to the proximal portion of the plunger rod upon the interior surface engaging the pair of extensions as the plunger rod translates distally relative to the flange by the first stroke while the pair of protrusions and the pair of slots are misaligned.

In some aspects, the techniques described herein relate to a drug delivery device, wherein the flange is configured to receive the pair of extensions in the pair of grooves upon the plunger rod rotating relative to the flange to align the pair of protrusions with the pair of slots.

In some aspects, the techniques described herein relate to a drug delivery device, wherein the pair of extensions are configured to expand radially outwards relative to the proximal portion upon the pair of grooves receiving the pair of extensions as the plunger rod rotates relative to the flange to align the pair of protrusions with the pair of slots.

In some aspects, the techniques described herein relate to a drug delivery device, wherein the pair of extensions are configured to translate distally through the pair of grooves in response to the plunger rod translating distally relative to the flange by the second stroke to position the pair of protrusions into the pair of slots.

In some aspects, the techniques described herein relate to a drug delivery device, wherein the flange includes a pair of openings formed in the proximal collar, the pair of openings are configured to at least partially receive the pair of extensions when the plunger rod is coupled to the flange prior to the pair of protrusions contacting the proximal collar to prime the drug delivery device.

In some aspects, the techniques described herein relate to a drug delivery device, wherein the flange is configured to inhibit proximal and rotational movement of the plunger rod relative to the body while the pair of extensions are received within the pair of openings; and wherein the pair of extensions are configured to exit the pair of openings upon distal movement of the plunger rod relative to the body to prime the drug delivery device.

In some aspects, the techniques described herein relate to a drug delivery device, wherein the body and the stopper are silicone-free such that an interior surface of the body and an exterior surface of the stopper each excludes a coating layer that includes silicone.

In some aspects, the techniques described herein relate to a drug delivery device, wherein the distal portion of the plunger rod includes a neck and a stem that is positioned distally of the neck, the plunger rod includes a cross-sectional shape that varies along a longitudinal length of the neck; and wherein the flange includes an opening that is configured to interface with the neck and the stem to control longitudinal and rotational movement of the plunger rod relative to the flange.

In some aspects, the techniques described herein relate to a drug delivery device, wherein the neck includes a first portion having a first cross-sectional shape that is configured to permit distal movement of the plunger rod relative to the flange and inhibit rotational movement of the plunger rod relative to the flange when the first portion is received within the opening.

In some aspects, the techniques described herein relate to a drug delivery device, wherein the neck includes a second portion that is positioned distally relative to the first portion, the second portion having a second cross-sectional shape that is configured to permit distal movement and rotational movement of the plunger rod relative to the flange when the second portion is received within the opening.

In some aspects, the techniques described herein relate to a drug delivery device, wherein the neck includes a third portion that is positioned distally relative to the second portion, the third portion having a third cross-sectional shape that is configured to permit distal movement of the plunger rod relative to the flange when the third portion is received within the opening and inhibit proximal movement of the plunger rod relative to the flange when the third portion is rotatably misaligned with the opening.

In some aspects, the techniques described herein relate to a drug delivery device, including: a body containing an aqueous pharmaceutical formulation comprising a VEGF antagonist; a plunger rod having a proximal end, a distal end contacting a stopper inside the body, and a pair of protrusions extending outwards from the proximal end; and a flange coupled to the body, the flange including a proximal collar and a pair of slots positioned along the proximal collar; wherein the flange is configured such that when the pair of protrusions and the pair of slots are misaligned with one another, distal movement of the plunger rod relative to the body is limited upon the pair of protrusions contacting the proximal collar to prime the drug delivery device by expelling air or at least a portion of the aqueous pharmaceutical formulation in the body; and wherein the flange is configured such that when the pair of protrusions and the pair of slots are aligned with one another, distal movement of the plunger rod relative to the body is limited upon the pair of protrusions extending into the pair of slots to deliver the VEGF antagonist from the body.

In some aspects, the techniques described herein relate to a drug delivery device, wherein the VEGF antagonist includes ranibizumab, bevacizumab, conbercept, pegaptanib, brolucizumab, aflibercept.

In some aspects, the techniques described herein relate to a drug delivery device, wherein the VEGF antagonist is aflibercept in a concentration of aflibercept of about 114.3 mg/mL.

In some aspects, the techniques described herein relate to a drug delivery device, wherein the VEGF antagonist is aflibercept and the aqueous pharmaceutical formulation further comprises a stabilizing agent and a buffering agent.

In some aspects, the techniques described herein relate to a drug delivery device, wherein the stabilizing agent includes one of arginine monohydrochloride, Sucrose, and Polysorbate 20; and wherein the buffering agent includes histidine.

In some aspects, the techniques described herein relate to a drug delivery device, wherein the body is configured to store a predetermined volume of the VEGF antagonist in a volume from about 150 μL to about 250 μL.

In some aspects, the techniques described herein relate to a drug delivery device, wherein the aqueous pharmaceutical formulation includes: about 100 mg/ml of a VEGF receptor fusion protein; about 10-100 mM L-arginine; sucrose; a histidine-based buffer; and a surfactant.

In some aspects, the techniques described herein relate to a drug delivery device, wherein the VEGF receptor fusion protein includes two polypeptides that each includes an immunoglobin-like (Ig) domain 2 of VEGFR1, an Ig domain 3 of VEGFR2, and a multimerizing component.

In some aspects, the techniques described herein relate to a drug delivery device, wherein the VEGF receptor fusion protein has less than about 3.5% high molecular weight species.

In some aspects, the techniques described herein relate to a drug delivery device, wherein the aqueous pharmaceutical formulation has a pH of about 5.0 to about 6.8.

In some aspects, the techniques described herein relate to a drug delivery device, wherein the aqueous pharmaceutical formulation comprises a VEGF receptor fusion protein having a mass ranging between about 2 mg to about 8 mg.

In some aspects, the techniques described herein relate to a drug delivery device, wherein the VEGF receptor fusion protein includes aflibercept.

In some aspects, the techniques described herein relate to a drug delivery device, wherein the aqueous pharmaceutical formulation comprises about 114.3 mg/ml of a VEGF receptor fusion protein as the VEGF antagonist, about 50 mM arginine monohydrochloride as the stabilizing agent, and about 10 mM histidine as the buffering agent.

In some aspects, the techniques described herein relate to a drug delivery device, wherein the aqueous pharmaceutical formulation includes: about 40 mg/ml VEGF receptor fusion protein; about 10 mM sodium phosphate; about 40 mM NaCl; about 0.03% polysorbate 20; about 5% sucrose; and a pH of about 6.2.

In some aspects, the techniques described herein relate to a drug delivery device, wherein the VEGF antagonist is prefilled within the body during an assembly of the body, the stopper, the plunger rod, and the flange of the drug delivery device such that the drug delivery device is a pre-filled syringe.

In some aspects, the techniques described herein relate to a packaging for housing a drug delivery device, including: a flexible tray and a removable cover adhered to the flexible tray, wherein the flexible tray includes: an opening; a cavity including a plurality of cavity portions; a plurality of sidewalls at least partially defining the cavity; a base at least partially defining the cavity; and a lip surrounding the opening and extending radially outwards from the cavity to define a periphery of the flexible tray, wherein the removable cover is adhered to the lip; wherein the flexible tray is configured to house the drug delivery device in the cavity with at least a portion of the drug delivery device contained within each of the plurality of cavity portions; and wherein the drug delivery device is a pre-filled syringe including an aqueous pharmaceutical formulation comprising a single dose of about 8 mg or more of a VEGF antagonist.

In some aspects, the techniques described herein relate to a packaging, wherein the plurality of cavity portions are connected to one another by narrowed portions, and each of the narrowed portions includes a first sidewall, a second sidewall, and a base that are collectively configured to receive at least a portion of a body of the drug delivery device.

In some aspects, the techniques described herein relate to a packaging, wherein one of the first sidewall, the second sidewall, or the base of each of the narrowed portions includes a geometric feature extending away from the first sidewall, the second sidewall, and the base and projecting into the narrowed portion.

In some aspects, the techniques described herein relate to a packaging, wherein the geometric feature is configured to abut against the portion of the body of the drug delivery device that is contained within the narrowed portion to position the body away from the first sidewall, the second sidewall, and the base of the narrowed portion.

In some aspects, the techniques described herein relate to a packaging, wherein the geometric feature is configured to suspend the body of the drug delivery device within the cavity, thereby forming a gap between an exterior of the body and the plurality of sidewalls and the base of the flexible tray.

In some aspects, the techniques described herein relate to a packaging, wherein the drug delivery device includes a body, a plunger rod, and a flange that are securely disposed inside the plurality of cavity portions and sealed inside the flexible tray by the removable cover closing the opening; and wherein the packaging and the drug delivery device are sterilized.

In some aspects, the techniques described herein relate to a packaging, wherein the removable cover is permeable to a gaseous sterilant that is configured to sterilize the flexible tray and the drug delivery device disposed inside the cavity during a sterilization of the packaging.

In some aspects, the techniques described herein relate to a packaging, wherein the flexible tray includes a projection positioned along the base and aligned with one of the plurality of cavity portions, wherein the projection extends outwards from the flexible tray in a direction opposite from the cavity.

In some aspects, the techniques described herein relate to a packaging, wherein the projection is hollow and open towards the cavity of the flexible tray.

In some aspects, the techniques described herein relate to a packaging, wherein the projection is configurable to be at least partially deformable in response to an application of force on the projection.

In some aspects, the techniques described herein relate to a packaging, wherein the projection is configured to flex the flexible tray in response to an application of force on the projection, thereby at least partially deforming one or more of the plurality of cavity portions and partially decoupling the drug delivery device from the cavity.

In some aspects, the techniques described herein relate to a packaging, wherein the projection is dome-shaped and a height of the projection from an apex of the projection to a base of the projection is greater than a distance between the base of the projection and the drug delivery device contained within the cavity and overlying the projection.

In some aspects, the techniques described herein relate to a packaging, wherein the projection includes a bulbous shape, and a diameter of the projection defines a widest portion of the bulbous shape where the projection meets the base of the flexible tray.

In some aspects, the techniques described herein relate to a packaging, wherein the projection includes a dome having a widest diameter at the base of the flexible tray, wherein the projection extends directly outwards from the base of the flexible tray such that the widest diameter of the dome is connected at the base.

In some aspects, the techniques described herein relate to a packaging, wherein the VEGF antagonist that is pre-filled in the drug delivery device includes aflibercept with a concentration of about 114.3 mg/mL.

In some aspects, the techniques described herein relate to a method of assembling a drug delivery device, including: filling a body of the drug delivery device with a substance; inserting a stopper of the drug delivery device into the body to fluidly seal the substance inside the body; coupling the body with a finger flange of the drug delivery device by inserting a proximal flange of the body into a lateral slot of the finger flange; inserting a plunger rod of the drug delivery device through the finger flange and into the body to an extent such that the plunger rod contacts the stopper; and coupling the plunger rod to the finger flange by inserting an extension of the plunger rod into a lateral opening of the finger flange.

In some aspects, the techniques described herein relate to a method, wherein filling the body with the substance includes: filling the body to a maximum target fill volume, wherein the maximum target fill volume includes a single dose having a predetermined volume that is less than the maximum target fill volume.

In some aspects, the techniques described herein relate to a method, wherein inserting the stopper into the body includes: forming a space within the body between the stopper and the substance as the stopper is inserted into the body, wherein the space defines an air pocket.

In some aspects, the techniques described herein relate to a method, wherein the space includes a headspace volume such that the maximum target fill volume of the substance and the headspace volume of the air pocket are collectively disposed between a distal end of the body and the stopper.

In some aspects, the techniques described herein relate to a method, wherein the substance includes a VEGF antagonist, the maximum target fill volume is between about 180 μL and about 250 μL, the predetermined volume of the single dose delivered from the drug delivery device is about 90 μL or less, and the body and the stopper are silicone-free.

In some aspects, the techniques described herein relate to a method, wherein inserting the stopper into the body includes: positioning the stopper near a proximal portion of the body that is adjacent to the proximal flange; and generating a vacuum pressure within the body to reposition the stopper from the proximal portion of the body towards a distal portion of the body that contains the substance, thereby sealing the substance in the body with the stopper.

In some aspects, the techniques described herein relate to a method, wherein the vacuum pressure generated within the body is to an extent such that the stopper is repositioned to a predetermined location within the distal portion of the body to maintain a maximum target fill volume of the substance that is pre-filled within the body.

In some aspects, the techniques described herein relate to a method, wherein coupling the body and the finger flange includes: engaging an upper surface of the proximal flange with a pair of ribs of the finger flange and a lower surface of the proximal flange with a lip of the finger flange as the proximal flange is inserted into the lateral slot.

In some aspects, the techniques described herein relate to a method, wherein the pair of ribs are at least partially deflectable in response to engaging the proximal flange, the pair of ribs being positioned inside the lateral slot of the finger flange.

In some aspects, the techniques described herein relate to a method, wherein coupling the body and the finger flange includes: engaging a side surface of the body with a pair of movable tabs of the finger flange as the proximal flange is inserted into the lateral slot.

In some aspects, the techniques described herein relate to a method, wherein the pair of movable tabs are at least partially deflectable in response to engaging the body, the pair of movable tabs being positioned outside the lateral slot of the finger flange.

In some aspects, the techniques described herein relate to a method, wherein inserting the plunger rod through the finger flange and into the body includes: applying an insertion force of about 15 N or less to the plunger rod for positioning a distal end of the plunger rod within the body adjacent to the stopper.

In some aspects, the techniques described herein relate to a method, wherein inserting the plunger rod through the finger flange and into the body includes: abutting a distal end of the plunger rod against a proximal end of the stopper without coupling the plunger rod to the stopper.

In some aspects, the techniques described herein relate to a method, wherein inserting the plunger rod through the finger flange and into the body includes: inserting a distal end of the plunger rod into a proximal end of the stopper without coupling the plunger rod to the stopper.

In some aspects, the techniques described herein relate to a method, wherein coupling the plunger rod to the finger flange includes: deflecting the extension of the plunger rod laterally inwards upon engaging a proximal edge of the finger flange as the plunger rod is inserted distally through the finger flange; and deflecting the extension laterally outwards upon entering the lateral opening of the finger flange as the plunger rod is inserted further distally through the finger flange.

In some aspects, the techniques described herein relate to a method of sterilizing a packaging assembly housing a drug delivery device, the method including: positioning the packaging assembly inside a sterilization chamber, wherein the drug delivery device housed within the packaging assembly is pre-filled with a VEGF antagonist; sterilizing the packaging assembly by conducting: (a) a primary sterilant exposure that includes: (i) adjusting a pressure in the sterilization chamber to a first sterilization pressure; (ii) injecting at least a first amount of a gaseous sterilant into the sterilization chamber; and (iii) injecting a first amount of a distribution gas into the sterilization chamber to raise the pressure in the sterilization chamber to a first transition pressure that is greater than the first sterilization pressure; (b) a secondary sterilant exposure that includes: (i) adjusting the pressure in the sterilization chamber to a second sterilization pressure; (ii) injecting at least a second amount of the gaseous sterilant into the sterilization chamber; and (iii) injecting a second amount of the distribution gas into the sterilization chamber to raise the pressure in the sterilization chamber to a second transition pressure that is greater than the second sterilization pressure.

In some aspects, the techniques described herein relate to a method, wherein each of the primary sterilant exposure and the secondary sterilant exposure comprises: directing the gaseous sterilant through a removable cover of the packaging assembly and into a cavity of the packaging assembly that houses the drug delivery device, wherein the drug delivery device includes a body that is silicone-free and stores the VEGF antagonist.

In some aspects, the techniques described herein relate to a method, wherein the first sterilization pressure and the second sterilization pressure each includes at least about 500 millibars, and the first transition pressure and the second transition pressure each include at least about 900 millibars.

In some aspects, the techniques described herein relate to a method, wherein the gaseous sterilant includes vaporized hydrogen peroxide; and wherein the first amount of the gaseous sterilant includes between about 125 grams to about 150 grams of the vaporized hydrogen peroxide, and the second amount of the gaseous sterilant includes between about 50 grams to about 75 grams of the vaporized hydrogen peroxide.

In some aspects, the techniques described herein relate to a method, wherein prior to sterilizing the packaging assembly, the method includes: conditioning the sterilization chamber by: (i) adjusting the pressure in the sterilization chamber to a conditioning pressure; and (ii) adding a conditioning gas into the sterilization chamber to raise the pressure in the sterilization chamber to at least about 150 millibar greater than the conditioning pressure.

In some aspects, the techniques described herein relate to a method, wherein the conditioning pressure is between about 450 millibars to about 650 millibars.

In some aspects, the techniques described herein relate to a method, wherein conditioning the sterilization chamber includes one of a plurality of conditioning pulses.

In some aspects, the techniques described herein relate to a method, wherein the first transition pressure is at least about 400 millibar greater than the first sterilization pressure, and the second transition pressure is at least about 400 millibar greater than the second sterilization pressure.

In some aspects, the techniques described herein relate to a method, further including: aerating the sterilization chamber by: (i) adjusting the pressure within the sterilization chamber to a first aeration pressure that is less than or equal to the first sterilization pressure; and (ii) adding an aeration gas into the sterilization chamber to raise the pressure in the sterilization chamber to an exhaust pressure that is greater than the first sterilization pressure.

In some aspects, the techniques described herein relate to a method, wherein the first aeration pressure includes between about 400 millibars to about 500 millibars, and the exhaust pressure includes between about 700 millibars to about 1000 millibars.

In some aspects, the techniques described herein relate to a method, wherein aerating the sterilization chamber further includes: adjusting the pressure within the sterilization chamber to a second aeration pressure over an exhaust time, wherein the second aeration pressure is equal to or greater than the first aeration pressure.

In some aspects, the techniques described herein relate to a method, wherein the second aeration pressure is at least 900 millibar, and the exhaust time is at least about 25 minutes.

In some aspects, the techniques described herein relate to a method, further including: sterilizing the packaging assembly by conducting a tertiary sterilant exposure that includes: (i) adjusting the pressure in the sterilization chamber to a third sterilization pressure; (ii) injecting at least a third amount of the gaseous sterilant into the sterilization chamber; and (iii) injecting a third amount of the distribution gas into the sterilization chamber to raise the pressure in the sterilization chamber to a third transition pressure that is greater than the second sterilization pressure.

In some aspects, the techniques described herein relate to a method, wherein the third transition pressure is at least about 400 millibar greater than the third sterilization pressure.

In some aspects, the techniques described herein relate to a method, wherein a total amount of the gaseous sterilant injected into the sterilization chamber during the primary sterilant exposure, the secondary sterilant exposure, and the tertiary sterilant exposure is greater than or equal to about 800 grams.

In some aspects, the techniques described herein relate to a method of treating or preventing an angiogenic eye disorder, including: administering a single initial dose of about 8 mg or more of a VEGF antagonist that is pre-filled in a first drug delivery device to an eye of a patient; administering one or more secondary doses of about 8 mg or more of the VEGF antagonist to the eye of the patient by one or more second drug delivery devices that are pre-filled with the VEGF antagonist; and administering one or more tertiary doses of about 8 mg or more of the VEGF antagonist to the eye of the patient by one or more third drug delivery devices that are pre-filled with the VEGF antagonist; wherein the first drug delivery device, the one or more second drug delivery devices, and the one or more third drug delivery devices each include a body storing the VEGF antagonist, a finger flange coupled to the body, and a plunger rod movably coupled to the finger flange for priming the body prior to administering the single initial dose, the one or more secondary doses, and the one or more tertiary doses.

In some aspects, the techniques described herein relate to a method, wherein the plunger rod of each of the first drug delivery device, the one or more second drug delivery devices, and the one or more third drug delivery devices includes a protrusion extending outwards from a proximal end of the plunger rod; and wherein the finger flange of each of the first drug delivery device, the one or more second drug delivery devices, and the one or more third drug delivery devices includes a proximal collar and a slot extending distally from the proximal collar.

In some aspects, the techniques described herein relate to a method, wherein the protrusion is configured to abut against the proximal collar upon the plunger rod moving distally relative to the finger flange to prime the body; and wherein the protrusion is configured to abut against the slot upon the plunger rod moving distally relative to the finger flange to administer the single initial dose, the one or more secondary doses, and the one or more tertiary doses.

In some aspects, the techniques described herein relate to a method, wherein the body of each of the first drug delivery device, the one or more second drug delivery devices, and the one or more third drug delivery devices is silicone-free.

In some aspects, the techniques described herein relate to a method, wherein the body of each of the first drug delivery device, the one or more second drug delivery devices, and the one or more third drug delivery devices is a syringe having a maximum volume of 0.5 mL or 1.0 mL.

In some aspects, the techniques described herein relate to a method, wherein the body of each of the first drug delivery device, the one or more second drug delivery devices, and the one or more third drug delivery devices includes a stopper that is silicone-free.

In some aspects, the techniques described herein relate to a method, wherein administering the one or more secondary doses includes: administering each of the one or more secondary doses about 2 weeks to about 4 weeks after an immediately preceding dose from the one or more secondary doses.

In some aspects, the techniques described herein relate to a method, wherein a time interval for administering each of the one or more secondary doses is based on an indication of the angiogenic eye disorder.

In some aspects, the techniques described herein relate to a method, wherein administering the one or more tertiary doses includes: administering each of the one or more tertiary doses about 4 weeks, about 8 weeks, or about 12 weeks after an immediately preceding dose from the one or more tertiary doses.

In some aspects, the techniques described herein relate to a method, wherein the VEGF antagonist includes aflibercept and the single initial dose, the one or more secondary doses, and the one or more tertiary doses have a volume of about 100 μL or less.

In some aspects, the techniques described herein relate to a method, wherein the first drug delivery device, the one or more second drug delivery devices, and the one or more third drug delivery devices are each configured to treat or prevent the angiogenic eye disorder by administering doses of aflibercept at an interval and a quantity whereby a clearance of free aflibercept from an ocular compartment in the eye is about 0.3 mL/day to about 0.46 mL/day after intravitreal injection of aflibercept to the eye.

In some aspects, the techniques described herein relate to a method, wherein a time for the clearance for free aflibercept to reach a lower limit of quantitation (LLOQ) in the ocular compartment of the eye after said intravitreal injection of aflibercept is about 15 weeks.

In some aspects, the techniques described herein relate to a method, wherein a time for free aflibercept to reach a lower limit of quantitation (LLOQ) in a plasma of the eye after said intravitreal injection of aflibercept is about 3.5 weeks to about 3.8 weeks.

In some aspects, the techniques described herein relate to a method, further including: administering one or more non-scheduled doses of the VEGF antagonists to the eye of the patient by one or more additional drug delivery devices that are pre-filled with the VEGF antagonist on a pro re nata basis in response to anatomic findings from the eye of the patient; wherein the one or more non-scheduled doses are capped at a minimum dosing frequency of one at least every 2 months to 4 months.

In some aspects, the techniques described herein relate to a method, wherein the angiogenic eye disorder includes one of age-related macular degeneration (AMD), macular edema (ME), retinal vein occlusion (RVO), diabetic macular edema (DME), neovascularization, neovascular glaucoma, post-surgical fibrosis in glaucoma, proliferative vitreoretinopathy (PVR), pannus, pterygium, vascular retinopathy, and diabetic retinopathy.

### BRIEF DESCRIPTION OF THE DRAWINGS

The accompanying drawings, which are incorporated into and constitute a part of this specification, illustrate various exemplary embodiments and, together with the description, serve to explain principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements in various embodiments, other than those specifically shown, are contemplated and are within the scope of the present disclosure.

There are many embodiments described and illustrated herein. The described devices and methods are neither limited to any single aspect nor embodiment thereof, nor to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein.

FIGS. 1A-IE depict an exemplary delivery device and components thereof, according to some embodiments of the present disclosure.

FIGS. 1F-2T depict additional aspects and embodiments of the exemplary delivery device of FIGS. 1A-IE.

FIGS. 3A and 3B depict an exemplary method of assembling the delivery device depicted in FIGS. 1A-IE, according to aspects of the present disclosure.

FIGS. 3C-3F depict an exemplary method of assembling an embodiment of the delivery device depicted in FIGS. 1A-IE, according to aspects of the present disclosure.

FIGS. 4A-4F depict an exemplary method of using the delivery device depicted in FIGS. 1A-1E, according to aspects of the present disclosure.

FIGS. 4G-4J depict an exemplary method of using an embodiment of the delivery device depicted in FIGS. 1A-IE, according to aspects of the present disclosure.

FIGS. 4K-4S depict exemplary aspects of plunger rods for use in embodiments of the delivery device depicted in FIGS. 1A-IE.

FIGS. 4T-4X depict views of an exemplary neck portion of a plunger rod and opening of a flange piece in embodiments of the delivery device depicted in FIGS. 1A-1E.

FIGS. 4Y-4Z depict exemplary aspects of plunger rods for use in embodiments of the delivery device depicted in FIGS. 1A-IE.

FIGS. 5A-5C depict another exemplary delivery device according to additional embodiments of the present disclosure.

FIGS. 6A-6E depict a further exemplary delivery device according to additional embodiments of the present disclosure.

FIGS. 7A-7F depict an exemplary method of using the delivery device depicted in FIGS. 6A-6E, according to aspects of the present disclosure.

FIGS. 8A-8E depict a further exemplary delivery device according to embodiments of the present disclosure.

FIGS. 8F and 8G depict a blocking component of the delivery device depicted in FIGS. 8A-8E according to embodiments of the present disclosure.

FIGS. 9A-9E depict an exemplary method of using the delivery device depicted in FIGS. 8A-8E, according to aspects of the present disclosure.

FIGS. 10A-10C depict an exemplary method of assembling the delivery device depicted in FIGS. 8A-8E, according to aspects of the present disclosure.

FIGS. 10D-10G depict another exemplary method of assembling a variation of the delivery device depicted in FIGS. 8A-8E, according to aspects of the present disclosure.

FIGS. 11A-11E depict an exemplary method of using the delivery device depicted in FIGS. 10D-10G according to aspects of the present disclosure.

FIGS. 12A-12D depict a close-up view of aspects of the exemplary method depicted in FIGS. 11A-11E.

FIGS. 13A and 13B depict a further exemplary delivery device and method of assembling said delivery device, according to additional embodiments of the present disclosure.

FIG. 14A-14F depict a method of using the delivery device depicted in FIGS. 12A and 12B.

FIGS. 15A-15B, and 16A-16B depict exemplary plunger rod dials according to further embodiments of the present disclosure.

FIG. 17 depicts an exemplary plunger rod and dial according to further embodiments of the present disclosure.

FIGS. 18A and 18B depict a further exemplary plunger rod and dial according to additional embodiments of the present disclosure.

FIGS. 19A and 19B depict an exemplary rotation lock mechanism according to additional embodiments of the present disclosure.

FIG. 20 depicts an exemplary plunger rod snap feature according to additional embodiments of the present disclosure.

FIG. 21 depicts an exemplary plunger rod with a bump feature according to additional embodiments of the present disclosure.

FIG. 22 depicts exemplary visual feedback features according to some embodiments of the present disclosure.

FIGS. 23A-23C depict a further exemplary delivery device according to aspects of the present disclosure.

FIGS. 24A-24E depict a further exemplary delivery device and method of using said delivery device, according to aspects of the present disclosure.

FIGS. 25A-25E depict a further exemplary delivery device and method of using said delivery device, according to aspects of the present disclosure.

FIGS. 26A-26G depict further exemplary delivery devices and a method of using one such delivery device, according to aspects of the present disclosure.

FIGS. 27A-27H depict a further exemplary delivery device and method of using said delivery device, according to aspects of the present disclosure.

FIGS. 28A-28Z depict further exemplary delivery devices and methods of using said delivery devices, according to aspects of the present disclosure.

FIGS. 29A-29C depict a further exemplary delivery device and method of using said delivery device, according to aspects of the present disclosure.

FIGS. 30-31 depict a further exemplary delivery device and method of using said delivery device, according to aspects of the present disclosure.

FIGS. 32-33 depict a further exemplary delivery device and method of using said delivery device, according to aspects of the present disclosure.

FIGS. 34-40C depict a further exemplary delivery device and method of using said delivery device, according to aspects of the present disclosure.

FIGS. 41A-41D depict exemplary flange pieces according to further embodiments of the present disclosure.

FIGS. 42A-42C depict an exemplary method of assembling the delivery device depicted in FIGS. 34-40C, according to aspects of the present disclosure.

FIG. 43 illustrates Mean Change from Baseline (BL) in Central Retinal Thickness (CRT; micrometers) over time through Week 44. Syringes on the X-axis indicate scheduled dose visits. Week 16 data for patients receiving treatment at week 16 are carried forward. LOCF: Patients receiving treatment at week 16 were considered not dry from week 16 onward. Inset box shows changes in CRT between weeks 12 and 20; 24 and 32; or 36 and 44 in the HD and IAI groups.

FIG. 44 illustrates Median Change from Baseline (BL) in Central Retinal Thickness (CRT; micrometers) over time through Week 44. Syringes on the X-axis indicate scheduled dose visit. LOCF: Patients receiving treatment at week 16 were considered not dry from week 16 onward.

FIG. 45 illustrates Mean Change from Baseline (BL) in Best Corrected Visual Acuity (BCVA; ETDRS letters) over time through Week 44. Syringes on the X-axis indicate scheduled dose visit. LOCF: Patients receiving treatment at week 16 were considered not dry from week 16 onward.

FIG. 46 depicts Ocular Treatment Emergent Adverse Events (TEAEs) in the Study Eye Occurring in ≥2% of Patients (Pts). Percentages and number of patients out of 53 (n/53) in HD and IAI groups are shown.

FIG. 47 depicts Ocular Serious Treatment Emergent Adverse Events (TEAEs) in the Study Eye. Percentages and number of patients out of 53 (n/53) in HD and IAI groups are shown.

FIG. 48 depicts a Summary of Intraocular Pressure Events in the HD and IAI Study Groups. Safety analysis set. Percentages are calculated based on N. Percentages and number of patients out of 53 (n/53) in HD and IAI groups are shown.

FIG. 49 depicts Non-Ocular Treatment Emergent Adverse Events (TEAEs) Occurring in ≥2% of Patients. Percentages and number of patients out of 53 (n/53) in HD and IAI groups are shown.

FIG. 50 depicts Non-Ocular Serious Treatment Emergent Adverse Events (TEAEs) in the HD and IAI study groups. Percentages and number of patients out of 53 (n/53) are shown.

FIG. 51 depicts Hypertension Treatment Emergent Adverse Events (TEAEs) in the HD and IAI study groups. Percentages and number of patients out of 53 (n/53) in HD and IAI groups are shown.

FIG. 52 depicts a dosing schedule and does regimen modification (DRM) criteria of PHOTON clinical trial.

FIG. 53 depicts criteria for dose regimen modifications of PHOTON clinical trial.

FIG. 54 depicts a CANDELA Clinical Trial Plan and Dosing Schedule according to embodiments of the present disclosure.

FIG. 55 depicts Proportion of Patients Losing and Gaining Vision (ETDRS letters) from Baseline (BL) at Week 44. Percentages and number of patients out of 53 (n/53) are shown.

FIG. 56 depicts Intraocular Inflammation Treatment Emergent Adverse Events (TEAEs) in the Study Eye. Percentages and number of patients out of 53 (n/53) in HD and IAI groups are shown.

FIG. 57 depicts Mean Pre-Dose Intraocular Pressure, Change from Baseline in the HD and IAI study groups over time (mmHg). Safety analysis set.

FIG. 58 provides a perspective view of exemplary packaging, according to an embodiment of the present disclosure.

FIG. 59 provides a bottom view of exemplary packaging containing a syringe, according to an embodiment of the present disclosure.

FIG. 60A provides a cross-sectional view of the packaging of FIG. 59 containing a syringe, according to an embodiment of the present disclosure.

FIG. 60B provides a cross-sectional view of the packaging of FIG. 59 containing a partially ejected syringe while in a flexed configuration, according to an embodiment of the present disclosure.

FIG. 61 provides a top view of exemplary packaging, according to an embodiment of the present disclosure.

FIG. 62 provides a perspective view of exemplary packaging, according to an embodiment of the present disclosure.

FIG. 63 provides a bottom view of exemplary packaging containing a syringe, according to an embodiment of the present disclosure.

FIG. 64 provides a perspective view of exemplary packaging, according to an embodiment of the present disclosure.

FIG. 65 provides a bottom view of exemplary packaging containing a syringe, according to an embodiment of the present disclosure.

FIG. 66 provides a bottom view of exemplary packaging containing syringes that are printed according to an embodiment of the present disclosure.

FIG. 67 provides a top view of exemplary packaging, according to an embodiment of the present disclosure.

FIG. 68 provides a bottom view of exemplary packaging, according to an embodiment of the present disclosure.

FIG. 69 provides a perspective view of exemplary packaging, according to an embodiment of the present disclosure.

FIG. 70 provides a side view of an exemplary packaging, according to an embodiment of the present disclosure. The dashed line identifies the cross-sectional view shown in FIG. 71.

FIG. 71 provides a cross-sectional view of exemplary packaging, according to an embodiment of the present disclosure. The cross-sectional view is taken from the dashed line shown in FIG. 70.

FIG. 72 provides a top view of exemplary packaging according to an embodiment of the present disclosure.

FIG. 73 provides a bottom view of exemplary packaging, according to an embodiment of the present disclosure.

FIG. 74 provides a perspective view of exemplary packaging according to an embodiment of the present disclosure.

FIG. 75 provides a side view of an exemplary packaging, according to an embodiment of the present disclosure. The dashed line identifies the cross-sectional view shown in FIG. 76.

FIG. 76 provides a cross-sectional view of an exemplary packaging, according to an embodiment of the present disclosure. The cross-sectional view is taken from the dashed line shown in FIG. 75.

FIGS. 77A-77B provide perspective views of exemplary packaging according to embodiments of the present disclosure.

FIG. 78A is a schematic drawing of an exemplary sterilization system that may be used for sterilization of medical products, such as the exemplary delivery devices according to aspects of the present disclosure.

FIG. 78B is another schematic drawing of an exemplary sterilization system that may be used for sterilization of medical products, such as the exemplary delivery devices according to aspects of the present disclosure.

FIG. 78C is a schematic drawing showing an expanded view of a portion of the system shown in FIG. 78A.

FIGS. 79A-79B are flow diagrams of steps in an exemplary method of sterilizing medical products using vaporized chemicals.

FIGS. 80A-80B are flow diagrams of steps in an exemplary method of performing a sterilization phase.

FIG. 81 is a flow diagram of steps in an exemplary method of performing an aeration phase.

FIG. 82 is a flow diagram of steps in an exemplary method of performing another aeration phase.

FIG. 83 is a flow diagram of steps in an exemplary method of sterilizing medical products using vaporized chemicals, according to aspects of the present disclosure.

FIG. 84 is a flow diagram of steps in an exemplary method of conditioning a sterilization apparatus, according to aspects of the present disclosure.

FIGS. 85A-85B are flow diagrams of steps in an exemplary method of exposing a sterilization load to vaporized sterilant, according to aspects of the present disclosure.

FIG. 86 is a flow diagram of steps in an exemplary method of aerating a sterilization apparatus, according to aspects of the present disclosure.

FIGS. 87-90B show sterilization chamber pressure and load temperatures during exemplary sterilization methods.

FIG. 91 is a plot showing a relationship between pressure and movement of a plunger in a prefilled syringe, according to aspects of the present disclosure.

FIG. 92 is a plot of pressure and temperature within an exemplary sterilization chamber during an exemplary sterilization method, according to aspects of the present disclosure.

There are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

### DETAILED DESCRIPTION

Embodiments of the present disclosure may be used in addition to and/or in combination with aspects of International Application No. PCT/US2018/021013, filed Mar. 6, 2018; International Application No. PCT/US2018/065192, filed Dec. 12, 2018; International Application No. PCT/US2020/036200, filed Jun. 4, 2020; International Application No. PCT/US2022/071436, filed Mar. 30, 2022; International Application No. PCT/US2022/029462, filed May 16, 2022; International Application No. PCT/US2023/061946, filed Feb. 3, 2023; International Application No. PCT/US2023/015223, filed Mar. 14, 2023; and U.S. Provisional Application No. 63/796,157, filed Apr. 28, 2025; each of which is incorporated by reference in its entirety herein.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Notably, an embodiment or implementation described herein as an “example” or “exemplary” is not to be construed as preferred or advantageous, for example, over other embodiments or implementations; rather, it is intended reflect or indicate the embodiment(s) is/are one “example,” rather than “ideal.” In addition, the terms “first,” “second,” and the like, herein do not denote any order, quantity, or importance, but rather are used to distinguish an element, a structure, a step or a process from another. Moreover, the terms “a” and “an” herein do not denote a limitation of quantity, but rather denote the presence of one or more of the referenced items. Similarly, terms of relative orientation, such as “front side, “top side,” “back side,” “bottom side,” “upper,” “lower,” etc. are referenced relative to the described figures. Additionally, the terms “about,” “approximately,” “substantially,” and the like, when used in describing a numerical value, denote a variation of +/−10% of that value, unless specified otherwise. It is completed that the terms “about,” “approximately,” “substantially,” and the like, are used interchangeably herein and intended to convey a similar numerical value and/or range of numerical values.

As used in the present disclosure, the term “sterilization” refers to achieving a level of sterility appropriate for a formulated drug substance or drug product for commercial distribution and use. Such a level of sterility may be defined in, for example, regulatory guidelines or regulations, such as guidelines released by the U.S. Food and Drug Administration. In some embodiments, such a level of sterility may include, for example, a 6-log, 9-log, and/or 12-log reduction in microbial populations of biological indicators placed on an outside or inside surface of a drug product (e.g., an outside surface of a syringe or an inside surface of a blister pack). In other embodiments, such a level of sterility may include, for example, a 9-log or 12-log reduction in microbial populations of biological indicators. Sterilization refers to achieving such an appropriate level of sterility while also achieving a sufficiently low level of residual sterilizing chemicals (e.g., vaporized hydrogen peroxide, ethylene oxide, etc.) for commercial distribution and use. Such a low level of residual sterilizing chemical may also be defined in regulatory guidelines or regulations.

As used in the present disclosure, the terms “terminal sterilization” and “external sterilization” refer to the sterilization of a drug product and/or drug delivery device in a container or packaging, such as in a primary packaging component, or in both primary and secondary packaging components, suitable for commercial distribution and use.

As used in the present disclosure, the term “medical product” refers to a product for medical use on a living animal. The term “medical product” includes, for example, drug products, formulated drug substances, medical implants, medical instruments, dose delivery devices, drug delivery devices, or combinations thereof. For example, the term “medical product” may refer to a syringe containing a formulated drug substance, such as a parenteral or an ophthalmic syringe. Other exemplary medical products include, e.g., suppository applicators and medication, transdermal drug delivery devices, medical implants, needles, cannulas, medical instruments, and any other product requiring sterilization prior to an intended medical use. In some examples, the term “medical product” may refer to one or more of the exemplary dose delivery devices and/or drug delivery devices depicted and described in the present disclosure.

As used in the present disclosure, the term “formulated drug substance” refers to a composition containing at least one active ingredient (e.g., a small molecule, a protein, a nucleic acid, or a gene therapy medicament) and an excipient, prepared for medical distribution and use. A formulated drug substance may include fillers, coloring agents, and other active or inactive ingredients.

As used in the present disclosure, the term “drug product” refers to a dosage form that contains a formulated drug substance, such as a finished dosage form for an active ingredient. A drug product may include packaging for commercial distribution or use, such as a bottle, vial, or syringe.

As used in the present disclosure, the term “vaporized chemical” refers to a chemical that has been converted into a substance that may be diffused or suspended in air. In some instances, a vaporized chemical may be a chemical that has been combined with water and then converted into a substance that may be diffused or suspended in air.

As used in the present disclosure, the term “fluid” refers to a liquid, semi-liquid, vapor, or gas including oxygen, hydrogen, nitrogen, or a combination thereof.

Embodiments of the present disclosure may be used with any type of fluid-containing products, such as liquid drug substances, liquid placebos, or other liquids that may be dispensed in a dose form. As used herein, the term “drug substance” may refer to a formulated substance including an active ingredient or ingredients, such as, e.g., small or large molecules, such as pain medications, steroids, or biologics. As used herein, the term “biologic” may refer to a large molecule (e.g., having a size greater than 15 kDa, greater than 30 kDa, greater than 50 kDa, greater than 75 kDa, or greater than 100 kDa) created in a living system such as a cell. Biologics may include proteins (e.g., antibodies), nucleic acids, large sugars, etc. Unlike small molecules that may have well-defined chemical structures, biologics may have highly complex structures that cannot be easily quantified by laboratory methods. As used herein, the term “drug product” may refer to a volume of a drug substance apportioned into a primary packaging component for packaging, transportation, delivery, and/or administration to a patient.

The term “primary packaging component” refers to a packaging component for a drug product, such as a drug container, that is designed and manufactured to be in direct physical contact with the formulated drug substance. (See, for example, Guidance for Industry on Container Closure Systems for Packaging Human Drugs and Biologics, U.S. Department of Health and Human Services, Food and Drug Administration, Center for Drug Evaluation and Research, and Center for Biologics Evaluation and Research (May 1999), which is incorporated by reference herein.) Examples of primary packaging components include pre-fillable syringes, Luer syringes, cartridges, and vials made of glass, plastic, other polymers or co-polymers, and/or other materials. In further examples, the primary packaging components may be made of a hybrid configuration in which one or more materials may be combined with one another, including but not limited to, a combination of glass and plastic.

As used herein, the terms “distal” and “distally” refer to a location (or portion of a device) relatively closer to, or in the direction of, a patient delivery site, and the terms “proximal” and “proximally” refer to a location (or portion of a device) relatively closer to, or in the direction of, a user end opposite a distal location/portion of a device.

As used herein, the term “body,” when used in reference to a part of a device, may refer to a component of the device suitable for containing a volume of a drug substance. A body may include, e.g., a barrel (such as a syringe barrel), tube, cylinder, or other containing portion of a device. In some embodiments, a body may also include a distal end portion having a nozzle, needle, needle attachment site, and/or distal cap.

### Dose Delivery Device & Medicament

Embodiments of the present disclosure may be used with products typically having small dose volumes, such as, e.g., ophthalmic drug products, vaccinations, insulin, etc. In some embodiments, devices of the present disclosure may be used with drug products including a large molecule, e.g., a molecular weight of 30 kDA or greater. In some embodiments, devices of the present disclosure may be used with drug products including a fragment of a large molecule. For example, in some embodiments, devices of the present disclosure may be used with drug products including an antigen-binding molecule. In some aspects, the antigen-binding molecule may be an antibody or antigen-binding fragment. In some embodiments, devices of the present disclosure may be suitable for use with drug products including ingredients such as, e.g., aflibercept, alirocumab, abicipar pegol, bevacizumab, brolucizumab, conbercept, dupilumab, evolocumab, tocilizumab, certolizumab, abatacept, rituximab, infliximab, ranibizumab, sarilumab, adalimumab, anakinra, trastuzumab, pegfilgrastim, interferon beta-1a, insulin glargine [rDNA origin], epoetin alpha, darbepoetin, filigrastim, golimumab, etanercept, antigen-binding fragments of any of the above, or combinations of such binding domains, such as a bispecific antibody to VEGF or angiopoietin-2, among others.

In some embodiments, devices and aspects of the present disclosure can be used with any therapies for ophthalmic diseases, including for the treatment of patients with Diabetic Eye Disease, post-injection noninfectious Endophthalmitis, Neovascular (Wet) Age-related Macular Degeneration (AMD), Macular Edema following Retinal Vein Occlusion (RVO), Diabetic Macular Edema (DME), and Diabetic Retinopathy (DR). In addition, in some embodiments, devices and aspects of the present disclosure can be used with various other therapies other than ophthalmic diseases including, but not limited to, vaccines, insulin, small dose treatments, or a combination thereof. In particular, large molecule and small molecule antagonists of VEGF and/or ANG-2, such as aflibercept, ranibizumab, bevacizumab, conbercept, OPT-302, RTH258 (brolocizumab), abicipar pegol (a pegylated designed ankyrin repeating protein (DARPin)), RG7716, or fragments thereof and in any concentration. Intravitreal (IVT) administration of therapeutic agents may be an effective treatment for such eye disorders (e.g., macular degeneration, retinal vein occlusion, macular edema, retinopathy, etc.), however, IVT administration includes various challenges such as drug product development, administration procedure and adverse events. For example, providing accurate and precise delivery of small volumes (10-100 μL) requires precise design of container components. Accordingly, inaccuracies in a dosage delivery (e.g., over or under-dosing) may provide undesired adverse events or lack of efficacy resulting in unpredictable and variable clinical responses.

In some embodiments, the medicament or drug substance stored within a drug delivery device of the present disclosure may conform to the US Pharmacopeia (USP) standard 789 (USP789) for particles within a solution.

In some embodiments, the drug product manufacturing process may include one or more of the following steps: (1) quality control inspection of drug product; (2) identity testing of drug product; (3) storage of drug product in a controlled environment (e.g., at a temperature of less than or equal to approximately 80 degrees Celsius); (4) thawing of drug product; (5) pooling and mixing of thawed formulated drug substance; (6) bioburden reduction filtration; (7) sterilizing filtration; (8) aseptic filling and stoppering of dose delivery device; (9) visual inspection; (10) time limits; and/or (11) shipping of dose delivery device. It should be appreciated that the manufacturing process may include additional and/or fewer steps than those described above without departing from a scope of this disclosure. Thawing the drug product may include removing one or more bottles from a freezer and thawing the bottles at room temperature. A thaw check may be performed throughout the thawing process as instructed by batch records. Thawing will be complete until no ice is visually present. A thaw time may be measured from when the bottles are removed from the freezer or other storage unit until thawing is complete. Thawed drug products may be held between approximately 2 degrees Celsius and 8 degrees Celsius until further processing and/or use. The thaw duration may be less than or equal to approximately 16 hours. The drug product may be transferred to cold storage (e.g., 2-8° C.) after thawing for a hold time until removal from the cold storage before pooling, such as for less than or equal to approximately 36 hours.

Pooling and mixing of the thawed formulation drug substance may take place within an environment that is under high efficiency particulate air (HEPA) filtered (unidirectional) airflow. Controls for pooling and mixing may include a pooled weight of approximately 9.8 kg to 31.8 kg, a mixing duration of approximately 30 minutes+/−10 minutes, and a mixing speed of approximately 250 rpm+/−20 rpm. The pooling hold time at ambient storage conditions may include approximately less than or equal to 24 hours from when the drug substance is removed from cold storage before pooling to the start of pooling.

The bioburden reduction filtration may be performed in an environment under HEPA filtered (unidirectional) airflow in which a pooled drug substance may be filtered using a 0.22 μm filter into a pre-sterilized single-use holding bag. Exemplary time limits for bioburden reduction filtration may include a filter flush volume (weight) of greater than or equal to approximately 90 mL (95.31 g); a flow rate of less than or equal to approximately 0.75 L/min; and a product-filter contact time for bioburden reduction and sterile filtration of less than or equal to approximately 53 hours. Exemplary performance parameters for bioburden reduction filtration may include a pre-BBR (“bioburden reduction”) filtration bioburden of approximately less than or equal to 10 CFU/100 mL, a pre-use FIT (“filter integrity test”) passing a threshold of greater than or equal to approximately 50 psi, and a post-use FIT passing a threshold of greater than or equal to approximately 50 psi.

The sterilization filtration may be performed using a redundant sterilizing filer assembly consisting of two 0.22 μm filters. Exemplary process parameters for sterilizing filtration may include a filter flush volume (weight) of greater than or equal to approximately 180 mL per filter to 190 mL per filter (190 g per filter to 95 g per filter); a flow rate of less than or equal to approximately 0.75 L/min (30 rpm); and a product-filter contact time for bioburden reduction and sterile filtration of less than or equal to approximately 53 hours. Exemplary performance parameters may include a pre-sterile filtration bioburden of less than or equal to approximately 10 CFU/100 mL; a pre-sterile filtration endotoxin of less than or equal to approximately 0.2 EU/mL; a filtration pressure of less than or equal to approximately 25 psi; a post-sterile filtration FIT (bubble point) passing a threshold of greater than or equal to approximately 50 psi; and a pre-sterile filtration solution temperature of greater than or equal to approximately 15° C. A pre-sterile filtration FIT, the upstream and downstream filters, may be measured for passing a threshold of greater than or equal to approximately 50 psi.

Aseptic time pressure filing of syringes may be performed in an enclosed/isolated environment under HEPA filtered (unidirectional) airflow. One or more exemplary fill machines may pick up a nest of approximately 100 dose delivery devices (e.g., syringes) from each tub of one or more tubs for placement on a table that is transported through the fill machine for filling and stoppering the syringes. Each of the syringes may be filled with a sterile filtered product and a weight check may be performed by the filling machine, such as for a minimum of approximately 2% of the filled syringes. Any syringes with fill weights outside of a segregation limit may be segregated and rejected for use. Consecutive weight checks may be performed for numerous syringes and the corresponding fill weights may be selectively adjusted until a predetermined number (e.g., at least three) are obtained. After the syringes are filled, stoppers may be inserted into the syringes via vacuum stopper insertion at a predetermined depth to allow for sufficient headspace disposed within the syringes. Once syringe filing, weight check, and stoppering is completed, the syringes may be set aside for visual inspection. The control parameters for aseptic filling and stoppering may include a fill weight between approximately 0.17 g to approximately 0.19 g, a fill duration of less than or equal to approximately 49 hours, and a stopper position within a body of the syringe of approximately 29.0 mm+/−10.0 mm. As described herein, the filling machine may be configured and operable to selectively locate the stopper within the body of the syringe at the predetermined position noted above by, for example, inserting the stopper into the syringe body via an insertion rod and generating a vacuum pressure within the syringe body to further move the stopper into position during the stoppering process.

As discussed above, a 100% visual inspection may be performed for each of the filled syringes, such as for identifying any defects relating to the particles, container closure, the drug product, and process handling. In some embodiments, a performance parameter that may be used for the visual inspection is in accordance with ASNI/ASQ Z1.4 (acceptance quality limit). Following visual inspection, the syringes may be bulk packaged and stored at approximately 2 degrees Celsius to 8 degrees Celsius prior to shipment for assembly, labeling, and secondary packaging.

Time limitations may be placed on the drug product manufacturing process, such as a duration from the end of thawing the drug product until the commencement of storage after filling, including after inspection and packaging, which may be referred to herein as a time out of refrigeration (“TOR”). For example, such time limit controls may include a total filter contact time of less than or equal to approximately 53 hours; a bulk TOR (e.g., end of thaw to start of storage) of less than or equal to approximately 139 hours; a DP TOR (e.g., end of thaw to start of storage after inspection) of less than or equal to approximately 201 hours; a post-thaw bulk between 2° C. to 8° C. of less than or equal to 36 hours; and a post-BBR filtration bulk between 2° C. to 8° C. of less than or equal to 36 hours.

Shipping procedures for packaging and labeling bulk drug products may include various measures, including but not limited to certain transportation modes (e.g., ground transportation via temperature-controlled truck), shipping temperatures between 2° C. to 8° C., and a maximum shipping duration of approximately 120 hours of continuous movement, such as when consistent shipping temperatures are not maintained. A load configuration during shipping may include a minimum load and a maximum load with respect to a number of bulk PFS contained within each tub, a number of tubs shipped per each shipper, and a total quantity of bulk PFS being transported. For example, a minimum load may include approximately 100 bulk PFS per tub, with approximately 8 tubs per shipper, and a total of approximately 800 bulk PFS. A maximum load may include 100 bulk PFS per tub, with approximately 8 tubs per shipper, and a total of approximately 12,800 bulk PFS.

In some embodiments, devices and aspects of the present disclosure may provide accurate dose delivery while also providing a container closure system for maintaining the agent in a sterile, stable, and safe condition to increase an intended shelf-life and efficacy of the agent. IVT drug products are primarily presented in glass vials, however, pre-filled syringes offer a more convenient administration by reducing the number of steps required for dose preparation. Preassembling the agent in the devices of the present disclosure may minimize the steps necessary for preparing a dose for delivery to a patient. Product development studies may focus on primary container component characterization, material compatibility with the formulation, formulation stability, fill volume determination, extractable/leachable and terminal sterilization.

Additionally, careful selection of ancillary components such as disposable syringes and needles, and a detailed administration procedure that includes dosing instructions can ensure successful administration of the product. Despite significant efforts in improving the drug product and administration procedures, ocular safety concerns such as endophthalmitis, increased intraocular pressure and presence of silicone floaters have been reported. Devices and aspects of the present disclosure may provide detailed administration procedures (e.g., priming instructions, dosing instructions, etc.) to ensure successful administration of the agent to a patient to minimize such ocular safety concerns. In some embodiments, devices and aspects of the present disclosure can also be used for cosmetic applications or medical dermatology, such as treatment or diagnosis of allergic responses.

In some embodiments, devices and aspects of the present disclosure can be used to perform various eye injection procedures, such as, for example, intraocular treatments and surgeries involving an intravitreal injection of a drug product. Devices and aspects of the present disclosure may be used to dispense drug products of varying protein concentration and/or viscosity, including, for example, drug products having a viscosity ranging from about 1 centipoise to about 10 centipoise, from about 2 centipoise to about 9 centipoise, from about 3 centipoise to about 8 centipoise, from about 4 centipoise to about 7 centipoise, or from about 5 centipoise to about 6 centipoise. Drug products having still other viscosities also are contemplated. Providing a precise dose with a device of the present disclosure may be important given a possible variability in protein concentration or viscosity of a drug product being delivered to a patient. Devices and aspects of the present disclosure may be further used to dispense varying volumes and/or quantities of a drug product, such as, for example, volumes ranging from about 1 μL to about 200 μL, from about 10 μL to about 190 μL, from about 50 μL to about 150 μL, from about 75 μL to about 125 μL, from about 90 μL to about 110 μL, or about 100 μL. Devices of the present disclosure may be configured and operable to require application of a minimum force exceeding a threshold for performing one or more procedures, such as, for example, priming a device, delivering a dosage, and the like. By requiring application of the minimum force, devices of the present disclosure may promote control in administering a consistent dose of a drug product, and promote safety by minimizing inadvertent movement of the device's components, thereby potentially reducing pain, discomfort, and injury to a patient.

In some embodiments, once the syringe is filled with the medicament, a stopper may be positioned within the syringe. For example, once the syringe is filled with the medicament, the stopper may be positioned approximately 29.0 mm+/−10.0 mm from the proximal end of the syringe. In other examples, the stopper may be positioned in a range of about 28.0 mm to about 31.0 mm+/−10.0 mm from the proximal end of the syringe. As described herein, a position of the stopper relative to the syringe body may be predetermined and/or selectively adjusted using a vacuum pressure that is generated within the syringe body after filling the medicament within the syringe body and prior to assembling of one or more of a plunger rod and a flange piece onto the syringe body.

As an example, a pre-filled 0.5 mL silicone-free syringe may include a nominal target placement of the stopper within the syringe body (i.e., the stopper height) of approximately 29.0 mm+/− approximately 10.0 mm relative to a proximal end of the syringe body, and a target fill weight for the fluid disposed therein ranging from approximately 0.15 g to approximately 0.25 g, such as, for example, approximately 0.18 g (0.17 mL). A measurement of the stopper placement may be performed during assembly of the pre-filled syringe with an insertion rod moving distally into the syringe body to a depth position ranging between approximately 430 mm to approximately 480 mm, such as, for example, approximately 456 mm+/−5.0 mm (i.e., in the z-direction), such as with a vacuum pressurization generated inside the syringe body within a range between approximately 50 mbar to approximately 100 mbar, such as, for example, approximately 80 mbar+/−5 mbar. In other words, vacuum assisted stoppering may be performed during assembly of the pre-filled syringe, in conjunction with the insertion rod advancing the stopper into the syringe body, to accurately position the stopper within the syringe body. In the example, the 0.5 mL silicone-free syringe may include a 0.5 mL silicone-free stopper with the target fill weight of approximately 0.18 g. In some examples, the 0.5 mL silicone-free syringe may include a 0.5 mL long luer lock cap, such as a TELC® Tip Cap or V-OVS®. It should be appreciated that a luer lock cap may be configured to twist off a body of the silicone-free syringe, and a tip cap may be configured to snap off the body of a syringe. In the embodiments, the silicone-free syringe includes a luer lock cap. It should be appreciated that the height of the stopper disposed within the syringe body is measured from a proximal (top) end of the syringe, such as adjacent to a proximal flange of the syringe body, towards the proximal end (i.e., the non-drug product contacting side) of the stopper.

In some embodiments, the maximum injection force of the syringe may be less than approximately 28 N, such as, for example, in a range between approximately 8 N to approximately 18 N, and by further example in a range from approximately 10 N to approximately 11 N. The contents of the device may be considered sterile when the sterility of the container is found to meet USP and/or Ph. Eur. Requirements. The endotoxin content may be in the range from less than approximately 0.05 EU/mL to less than 0.15 EU/mL.

In some embodiments, devices according to the present disclosure may be manufactured and/or otherwise prepared to be completely silicone free, such that a body (e.g., a vial, a syringe, a cartridge, etc.) storing a substance (e.g., a drug, a medicament, etc.) may not include any amounts of silicone, such as along an interior surface defining a lumen of the body. In other embodiments, devices according to the present disclosure may be manufactured and/or otherwise prepared to be substantially (e.g., 90% or greater) silicone free. In further embodiments, devices according to the present disclosure may be manufactured and/or otherwise prepared to include plasma-cured silicone oil or baked-on silicone, such as along the interior surface defining the lumen of the body. In such instances, it should be appreciated that an amount of baked-on silicone is relatively minimal.

In further embodiments, a maximum injection force of the pre-filled silicone-free syringe with approximately 8 mg of a drug substance, such as a VEGF antagonist (e.g., aflibercept), may include a minimum of approximately 13 N to approximately 14 N, a maximum of approximately 17 N to approximately 18 N, and an average of approximately 15 N to approximately 16 N with a standard deviation of approximately 0.8 N.

In other embodiments, a delivered volume from the pre-filled silicone-free syringe may include an average delivered volume ranging from approximately 0.08 mL to 0.09 mL with a standard deviation ranging from approximately 0.001 mL to approximately 0.002 mL. In some examples, the silicone-free syringe may be pre-filled with approximately 8 mg of a drug product, such as a VEGF antagonist, with a stoppering pressure ranging from approximately 40 mbar to approximately 50 mbar.

In further embodiments, a finger flange of the pre-filled silicone-free syringe may be configured to receive a maximum force during assembly with the syringe body. For example, a flange of a syringe body by Gerresheimer (GH) may be configured to receive an average maximum force ranging from approximately 51 N to approximately 53 N with a standard deviation of approximately 4 N. As a further example, a flange of a syringe body by Ompi may be configured to receive an average maximum force of approximately 42 N to approximately 44 N with a standard deviation of approximately 6 N.

In further embodiments, a tip cap (e.g., needle attachment portion, such as TELC® Tip Cap) of the pre-filled silicone-free syringe may be configured to receive a torque, such as by twisting/rotating at a rate of approximately 19 rev/minute to approximately 23 rev/minute, for detaching the tip cap from the syringe body during use. For example, a minimum tip cap torque may range between approximately 6 N*cm to approximately 7 N*cm, a maximum tip cap torque may range between approximately 9 N*cm to approximately 10 N*cm, and an average tip cap torque may range between approximately 7 N*cm to approximately 8 N*cm with a standard deviation of approximately 0.5 N*cm.

In other embodiments, the pre-filled silicone-free syringe may include a dead volume distribution disposed inside the syringe body between a distal end (fluid contacting side) of the stopper and a distal end of the syringe body. For example, a minimum dead volume distribution included within the body of the pre-filled silicone-free syringe may be approximately 0.01 mL, a maximum dead volume distribution may be approximately 0.05 mL, and an average dead volume distribution may be approximately 0.03 mL with a standard deviation of approximately 0.01 mL. Numerous volumes may collectively define the dead volume distribution, including but not limited to, a fill volume, an expelled volume, a total dead volume, a needle dead volume, and a syringe dead volume. In this instance, exemplary volumes measured inside the syringe body may include, for example, a fill volume of approximately 0.17 mL with a standard deviation of approximately 0.001 mL, an expelled volume of approximately 0.13 mL with a standard deviation of approximately 0.01 mL, a total dead volume of approximately 0.03 mL with a standard deviation of approximately 0.11 mL, a needle dead volume of approximately 0.01 mL with a standard deviation of approximately 0.01 mL, and a syringe dead volume of approximately 0.02 mL with a standard deviation of approximately 0.01 mL.

In some embodiments, a measurement of the stopper and a delivered volume of drug product (e.g., VEGF antagonist) from the pre-filled silicone-free syringe may be selectively determined such that the delivered volume of a dose is relatively independent of a fill volume. For example, when the silicone-free syringe is pre-filled with approximately 174 μL of 8 mg of the drug product (e.g., aflibercept), an average delivered volume may be approximately 0.08 mL with a standard deviation of approximately 0.003 mL. In another example, the average delivered volume may be approximately 0.09 mL with a standard deviation of approximately 0.002 mL. A distribution of the stopper position within the syringe body may include a minimum position ranging between approximately 27 mm to approximately 29 mm, a maximum position ranging between approximately 29 mm to approximately 31 mm, and an average position ranging between approximately 29 mm to approximately 30 mm with a standard deviation of approximately 0.4 mm. A distribution of the delivered volume from the syringe body may include a minimum volume of approximately 0.07 mL, a maximum volume of approximately 0.09 mL, and an average volume of approximately 0.08 mL with a standard deviation of approximately 0.003 mL.

In some embodiments, a compressed length of the stopper and a delivered volume of the pre-filled silicone-free syringe may be selectively determined and/or adjusted. For example, the pre-filled silicone-free syringe may include a target fill of approximately 0.2 g+/− approximately 0.01 g and a target position of the stopper within the syringe body of approximately 29.0 mm+/− approximately 10.0 mm. In the example, the compressed length of the stopper may include a minimum length of approximately 7.4 mm+/− approximately 0.5 mm, a maximum length of approximately 7.5 mm+/− approximately 0.5 mm, and an average length of approximately 7.5 mm+/− approximately 0.5 mm with a standard deviation of approximately 0.03 mm. The position of the stopper within the syringe body may include a minimum position of approximately 28 mm+/− approximately 10 mm, a maximum position of approximately 30 mm+/− approximately 10 mm, and an average position of approximately 29 mm+/− approximately 10 mm with a standard deviation of approximately 0.1 mm. The deliverable mass of the drug product stored in the syringe body may include a minimum mass of approximately 0.08 g+/− approximately 0.05 g, a maximum mass of approximately 0.09 g+/− approximately 0.05 g, and an average mass of approximately 0.084 g with a standard deviation of approximately 0.002 g.

In some embodiments, an axial pull-off force for decoupling a luer lock adapter collar from the pre-filled silicone-free syringe may be selectively determined, in which the tip cap (e.g., TELC® Tip Cap) is coupled to the luer lock adapter collar. In this instance, an instrument (e.g., a chuck) may be inserted between the luer lock adapter collar and a body of the pre-filled silicone-free syringe to remove the luer lock adapter collar from the body. For example, a minimum axial pull-off force may range from approximately 70 N to approximately 100 N, a maximum axial pull-off force may range from approximately 140 N to 170 N, and an average axial pull-off force may range from approximately 120 N with a standard deviation of approximately 10 N to approximately 140 N with a standard deviation of approximately 8 N.

In further embodiments, a fill height of the drug product stored in the pre-filled silicone-free syringe may be selectively determined relative to the proximal (top) end of the syringe flange to a bottom of the meniscus. For example, a minimum fill height may be approximately 37.34 mL, a maximum fill height may be approximately 39.22 mL, and an average fill height may be approximately 38.53 mL with a standard deviation of approximately 0.43 mL.

In further embodiments, the pre-filled silicone-free syringe may be configured to include a seal integrity between the syringe body and the stopper. In this instance, the syringe body may be filled with the drug product (e.g., VEGF antagonist) to a nominal capacity that requires expelling at least a portion of air disposed therein. For example, an axial force applied to the plunger rod may force the stopper distally to generate a pressure of approximately 300 kPa and maintain such pressure for approximately 30 seconds. A minimum force during such application may range from approximately 5 N to approximately 7 N for approximately 30 seconds to approximately 40 seconds, with a maximum force of approximately 5 N for approximately 40 seconds.

In additional embodiments, a position of the stopper relative to the syringe body of the pre-filled silicone-free syringe and a delivered mass from the syringe body may be selectively determined and/or adjusted relative to one another. In this instance, the position of the stopper within the syringe body may be measured from a proximal end of the syringe flange to the proximal (top) end of the stopper. For example, the position of the stopper may include a minimum position of approximately 27.7 mm, a maximum position of approximately 29.4 mm, and an average position of approximately 28.7 mm with a standard deviation of approximately 0.4 mm. By further example, the delivered mass may include a minimum mass of approximately 0.07 g, a maximum mass of approximately 0.09 g, and an average mass of approximately 0.08 g with a standard deviation of approximately 0.002 g.

In additional embodiments, a delivered volume of the drug product (e.g., VEGF antagonist) from the pre-filled silicone-free syringe having a target fill volume of approximately 174 μL may include a minimum volume of approximately 0.07 mL, a maximum volume of approximately 0.09 mL, and an average volume of approximately 0.08 mL with a standard deviation of approximately 0.002 mL.

In some embodiments, 0.5 mL pre-filled silicone-free syringes may be packaged in a 100-count nest configuration, as described in detail above. An exemplary syringe filling machine may utilize 0.5 mL standard change parts to handle the 0.5 mL standard nest configuration. In some examples, each of the 0.5 mL syringes may include an inner diameter ranging between approximately 4.5 mm to approximately 4.8 mm, an outer diameter ranging between approximately 6.7 mm to approximately 7.0 mm, and a total length ranging between approximately 66 mm to approximately 69 mm (max). The 0.5 mL stoppers may include a total height ranging between approximately 5.7 mm to approximately 6.3 mm and an outer neck diameter ranging between approximately 4.8 mm to approximately 5.2 mm. It should be appreciated that the outer neck diameter may correspond to a diameter of the stopper at an outer rib of the stopper.

Dose delivery devices available on the market, such as pre-filled syringes or syringes for use with vials, may not necessarily assist with accurately loading a desired volume of a substance, priming the devices, expelling an excessive volume of drug substance from the devices, and/or removing air bubbles from the devices. In dose delivery devices containing a small volume of a drug substance in particular (e.g., about 500 μL or less, about 300 μL or less, about 250 μL or less, about 200 μL or less, about 150 μL or less, about 100 L or less, about 50 μL or less, or about 25 μL or less, such as between about 25 μL and about 50 μL, between about 50 μL and about 100 μL, between about 25 μL and about 100 L, between about 50 μL and about 150 μL, between about 100 μL and about 250 μL, between about 100 μL and about 150 μL, between about 150 μL and about 250 μL, between about 200 μL and about 250 μL, between about 200 μL and about 500 μL, or between about 250 μL and about 500 L), it may also be difficult to confirm the presence of the correct dose of a drug substance in the device with the naked eye. Currently in the dose delivery device market, and specifically in the syringe market, there is a need for mechanisms that allow a user to set precisely for delivery a small volume of a product in a syringe (e.g., a pre-filled or fillable/refillable syringe), prime the syringe, remove air bubbles from the syringe, and/or confirm or be assured that the dose volume in the syringe is correct. Embodiments of the present disclosure may assist manufacturers, drug product providers, medical professionals, and/or patients with accurately making, filling, or otherwise preparing a dose administration device, priming the device, removing bubbles from the device, confirming the dose, and/or administering a dose from the device to a patient. Moreover, embodiments of the present disclosure may assist in preventing or mitigating errors or variation in device manufacture or use, such as errors or variation in placement of dose lines on devices, variation in device geometry (e.g., variation in syringe neck geometry), variations in component manufacturing tolerance, and/or variation or errors in setting a dose line prior to delivery of a product.

In some instances, embodiments of the present disclosure may be of particular assistance to individuals who may have difficulty setting doses with precision and accuracy. For example, embodiments of the present disclosure may assist elderly individuals, young children, or persons with physical or mental disabilities in setting accurate doses.

Described herein are various embodiments of dose delivery devices, and in particular, for syringes. In some instances, embodiments or aspects of embodiments disclosed herein may be used in conjunction with existing syringe body parts to modify off-the-shelf products, which may reduce the development and manufacturing time for the dose delivery devices. In other instances, embodiments or aspects of embodiments disclosed herein may be included in devices during their manufacture. The syringes described herein may be pre-filled or may be fillable/refillable.

Embodiments of the present disclosure may include syringes having rotating parts, threaded parts, springs, gears, detents, channels, grooves, and the like, that may allow a user to precisely control the movement of priming and dosage delivery elements such as, e.g., plungers and/or stoppers. Such parts may be intended to reduce human error and/or increase accuracy.

In some embodiments, visualization devices, such as magnifiers, may be provided with, attached to, or otherwise disposed on, delivery devices, in order to help enhance visibility of dose measurement markers on the devices. It is contemplated that aspects of one embodiment (such as sleeves, channels, blocking components, protrusions, detents, threaded parts, grips, visual, tactile, or auditory indicators, etc.) may be combined with aspects of one or more other embodiments, to create various combinations and permutations of features in a single device.

In some embodiments, devices according to the present disclosure may be depicted as including one type of plunger rod and plunger, or as including a general schematic representation of a plunger rod and plunger. For example, some devices according to the present disclosure may be depicted or described as including, e.g., a plunger rod having a ball-tipped end (e.g., non-threaded), which engages with a stopper such that the plunger rod and the stopper may be attached together. It is contemplated that multiple and/or different configurations of plunger rods and stoppers may be appropriate for each of the embodiments disclosed herein. For example, in some cases, the aforementioned ball-tipped plunger rod may be used with embodiments disclosed herein. In some embodiments, a plunger rod may not be affixed to a stopper, and instead may be disposed near, next to, or flush against a stopper such that pressure from the plunger rod towards the stopper may push the stopper, but withdrawal, twisting, or other movement of the plunger rod may not cause the stopper to likewise be withdrawn, twisted, or otherwise moved. As another example, in some embodiments, a plunger rod may be affixed to a stopper by threads, a clip, or an adhesive, or may be of a single piece with a stopper (e.g., may have been manufactured in a single mold with a stopper).

In some embodiments, devices according to the present disclosure may include various cosmetic features relevant to intended users of the devices. For example, devices according to the present disclosure may be manufactured and sold for use with pediatric, elderly, or differently-abled patients. In such cases, devices according to the present disclosure may include child-friendly coloring, cartoon images, or other cosmetic features to appeal to children, or high-contrast coloring, textured surfaces, or other features to enhance ease of identification and/or use. In some cases, devices according to the present disclosure may include lettering, labeling, or other features designed to be easily recognized by the intended users. For example, lettering on a pediatric device or a device for use by a disabled or differently-abled person or an elderly person may have larger, more accessible labeling so that it may be more easily recognized and read by the user(s) of the device. In some embodiments, lettering or labeling may be raised, molded, or embossed. In some embodiments, the drug delivery device (DDD), includes a body; a plunger rod disposed partially inside the body; a protrusion extending from the plunger rod; and a blocking component coupled to a proximal end portion of the body, wherein the blocking component is a flange piece, wherein, when the protrusion is in a first position relative to the blocking component, the blocking component restricts distal movement of the plunger rod to a first stopping point, and when the protrusion is in a second position relative to the blocking component, the blocking component restricts distal movement of the plunger rod to a second stopping point. In an embodiment of the invention, the DDD further includes: a stopper disposed in the body, wherein distal movement of the plunger rod distally moves the stopper; and a drug substance disposed in the body in between the stopper and a distal end of the body, wherein distal movement of the plunger rod to the first stopping point primes the drug delivery device, and distal movement of the plunger rod to the second stopping point dispenses a predetermined volume of the drug substance from a distal end of the device.

In some instances, moving the protrusion from the first position to the second position includes twisting the plunger rod relative to the blocking component. In an embodiment of the invention, the DDD further includes: a cavity in a proximal side of the blocking component, the cavity sized and configured to receive a portion of the protrusion, wherein when the protrusion is in the second position relative to the blocking component, the protrusion is positioned proximally from the cavity, such that distal movement of the plunger rod moves the protrusion into the cavity; e.g., wherein the cavity is a first cavity, and further includes: a second cavity in a proximal side of the blocking component, the second cavity sized and configured to receive a portion of the protrusion, wherein the first and second cavity are located on opposite sides of a central longitudinal axis of the drug delivery device. In an embodiment of the invention, the plunger rod passes through an opening in the blocking component.

In some instances, the DDD further includes an actuation portion at a proximal end portion of the plunger rod, wherein the protrusion extends from the actuation portion, e.g., wherein the actuation portion includes a generally cylindrical shape having a diameter greater than a width of the remainder of the plunger rod, wherein the protrusion extends from a side of the generally cylindrical shape, and wherein the actuation portion further comprises: a thumb pad on a proximal end of the actuation portion; and a ring on an exterior surface on the side of the generally cylindrical shape; e.g., further including a proximal collar on the blocking component, wherein the actuation portion partially fits inside the proximal collar; e.g., wherein the plunger rod further includes a pair of extensions protruding distally from the actuation portion and the blocking component (e.g., which includes one or more indents formed along a bottom wall of the blocking component; and wherein a portion of each extension is configured to be received by the one or more indents upon distal movement of the plunger rod relative to the blocking component to allow distal movement of the plunger rod to the second stopping point; or, which includes one or more indents formed along a bottom wall of the blocking component.

In some instances, a portion of each extension is configured to be received by the one or more indents upon distal movement of the plunger rod relative to the blocking component to allow distal movement of the plunger rod to the second stopping point; or, which includes a pair of internal grooves formed along a sidewall of the blocking component; and wherein a portion of each extension is configured to be received by at least one of the pair of internal grooves upon rotation of the plunger rod relative to the blocking component to expand the extensions radially-outward from a compressed state to a relaxed state) includes a pair of openings; and wherein a portion of each extension is configured to be received by one of the pair of openings in the first stopping point.

In some instances, the protrusion is a first protrusion, and further includes a second protrusion extending from the plunger rod in a direction opposite to the first protrusion. In an embodiment of the invention, the blocking component is slidably coupled to the body and includes a third cavity and a pair of ribs that extend into the third cavity, wherein the body includes a top flange and the pair of ribs are configured to engage the top flange received in the third cavity; wherein the pair of internal ribs are configured to apply a distally-directed force onto the top flange. In an embodiment of the invention, the blocking component is slidably coupled to the body and includes a pair of movable tabs that are configured to engage the body; and the pair of movable tabs are laterally deflectable upon receiving the body in the blocking component and are configured to apply a radially-inward directed force onto the body. In some instances, the blocking component further includes a pair of finger flanges, and each of the finger flanges includes a textured surface having a predefined pattern that increases a grip of the blocking component.

In further embodiments, a drug delivery device (DDD) includes a body; a plunger rod having a distal end contacting a stopper inside the body, and a proximal end including an actuation portion with a thumb pad; a plurality of protrusions extending from the actuation portion; and a blocking component disposed on the body, the blocking component including a proximal collar having a plurality of slots.

In some instances, when the protrusions and the slots are in a first configuration relative to one another, the blocking component restricts distal movement of the plunger rod to a first stopping point, and when the protrusions and the slots are in a second configuration, the blocking component restricts distal movement of the plunger rod to a second stopping point, wherein, in the second configuration, the slots are configured to receive the protrusions upon distal movement of the plunger rod. In some instances, the protrusions and the slots are movable from the first configuration to the second configuration by rotation of the actuation portion about a longitudinal axis in relation to the blocking component, and wherein when the protrusions and the slots are in the second configuration, the protrusions and the slots are not movable to the first configuration; and/or a difference between the first stopping point and the second stopping point is equivalent to a distance that the stopper must travel to expel a predetermined volume of a drug product from a distal end of the body, wherein the plunger rod is prevented from moving from the second stopping point to the first stopping point.

In some instances, the plurality of protrusions includes two protrusions disposed symmetrically about the actuation portion; and/or the blocking component further comprises a pair of finger flanges; and/or the drug delivery device is a pre-filled syringe; and/or the drug delivery device is changeable: (a) from a pre-use state to a primed state, by longitudinally moving the plunger rod (e.g., wherein the plunger rod includes a neck disposed distally from the actuation portion, wherein the neck interfaces with an opening in the blocking component to prevent proximal movement of the plunger rod, for example, wherein the neck further interfaces with the opening in the blocking component to prevent movement of the drug delivery device from the delivery state to the primed state) until the plunger rod reaches the first stopping point; (b) from the primed state to a delivery state by rotating the plunger rod in relation to the blocking component until the protrusions and the blocking component are in the second configuration; and (c) from a delivery state to a used state by longitudinally moving the plunger rod until the plunger reaches the second stopping point, wherein the drug delivery device is not changeable from the used state to the delivery state, from the delivery state to the primed state, or from the primed state to the pre-use state. In some instances, when the plunger rod is at the second stopping point, the stopper does not contact a distal end of the body.

In further embodiments, a drug delivery device includes a body; a plunger rod, including a distal portion contacting a stopper inside the body; a proximal end including a generally cylindrical actuation portion disposed outside of the body; and two protrusions extending from opposite sides of the actuation portion in a symmetrical configuration; and a blocking component coupled to the body, the blocking component including a collar configured to accept a distal part of the actuation portion; and two cavities in the collar having proximally-facing openings, wherein each cavity is configured to accept a distal portion of one of the two protrusions.

In some instances, the plunger rod is longitudinally movable and rotatable about a longitudinal axis relative to the blocking component, and when the drug delivery device is in a pre-use state, the protrusions and the cavity openings are not longitudinally aligned, and when the drug delivery device is in a delivery state, the protrusions and the cavity openings are longitudinally aligned. In some instances, the blocking component further includes a finger flange, and further includes a ribbed surface on a side of the actuation portion. In some instances, the plunger rod further includes: two extensions protruding distally from the actuation portion; and a plurality of openings in the collar of the blocking component, wherein a portion of each extension is configured to be received by one of the plurality of openings upon distal movement of the plunger rod relative to the blocking component.

In some embodiments, a substance may be dispensed using a DDD having a plunger rod and a body, by a method including (a) advancing the plunger rod by a predetermined distance into the body until advancement of the plunger rod is resisted by a stop; (b) rotating the plunger rod about a longitudinal axis; and (c) actuating the plunger rod to dispense a predetermined volume of the substance, wherein none of steps (a), (b), and (c) are reversible.

In some embodiments, the DDD further includes a flange piece having a collar, and advancing the plunger rod and actuating the plunger rod comprise pressing an actuation portion of the plunger rod into the collar of the flange piece; for example, wherein the plunger rod comprises a protrusion, and wherein the collar of the flange piece abuts against the protrusion to resist advancement of the plunger rod. For example, in an embodiment of the invention, wherein rotating the plunger rod comprises twisting an actuation portion of the plunger rod relative to the flange piece, until a protrusion on the plunger rod becomes longitudinally aligned with a cavity in the collar of the flange piece, which may further include advancing the protrusion into the cavity until the protrusion abuts a distal side of the cavity, wherein the predetermined volume of the substance is dispensed when the protrusion abuts the distal side of the cavity. See International patent application publication no. WO2020/247686.

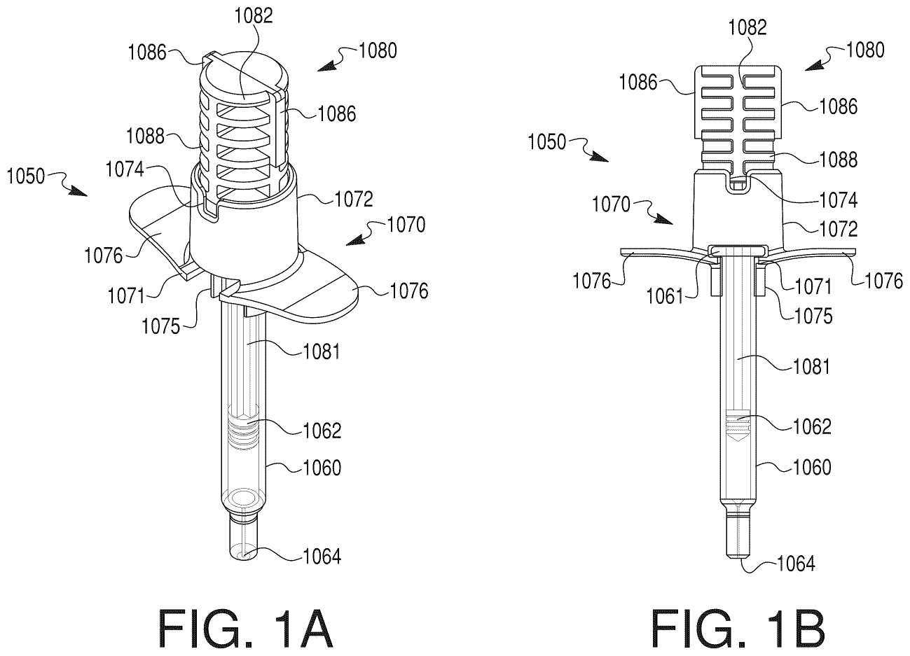

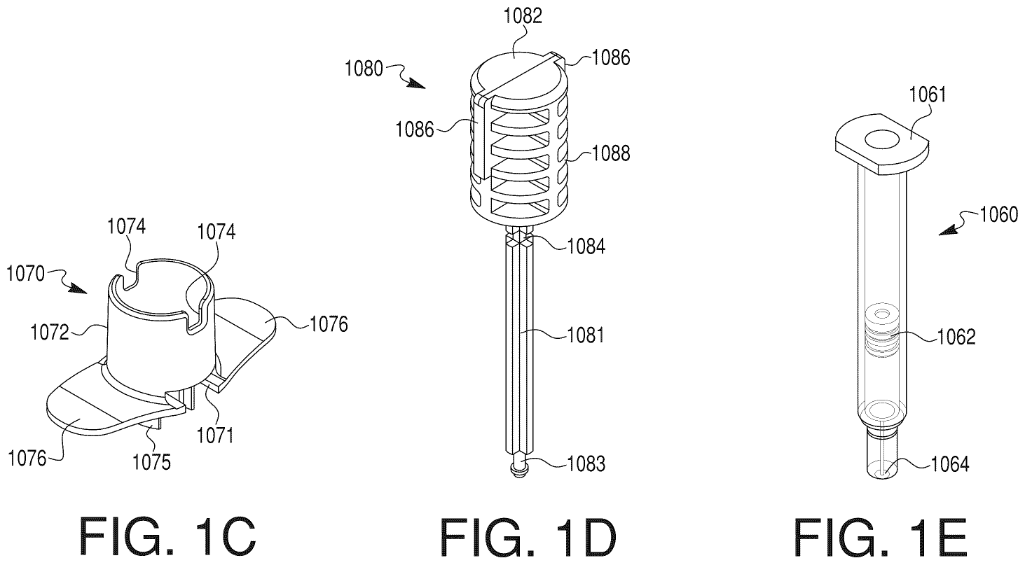

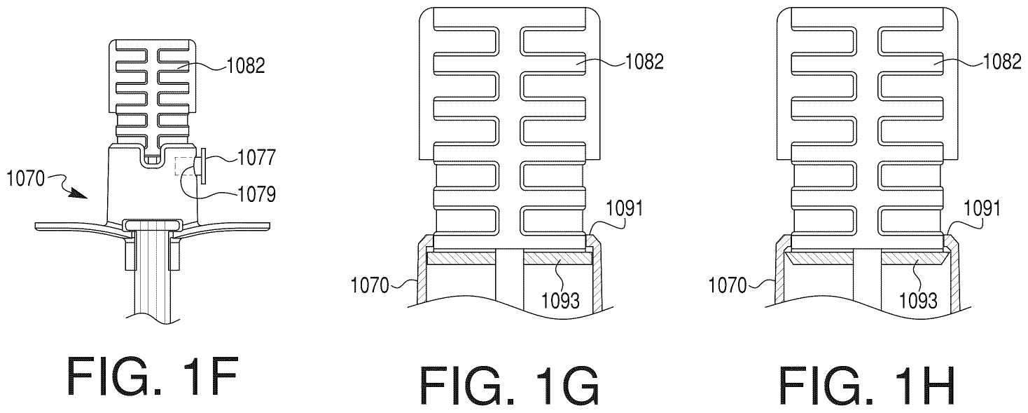

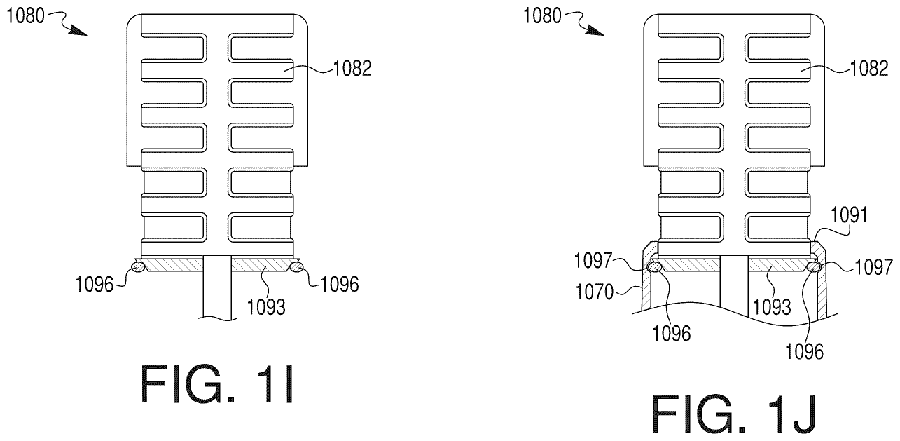

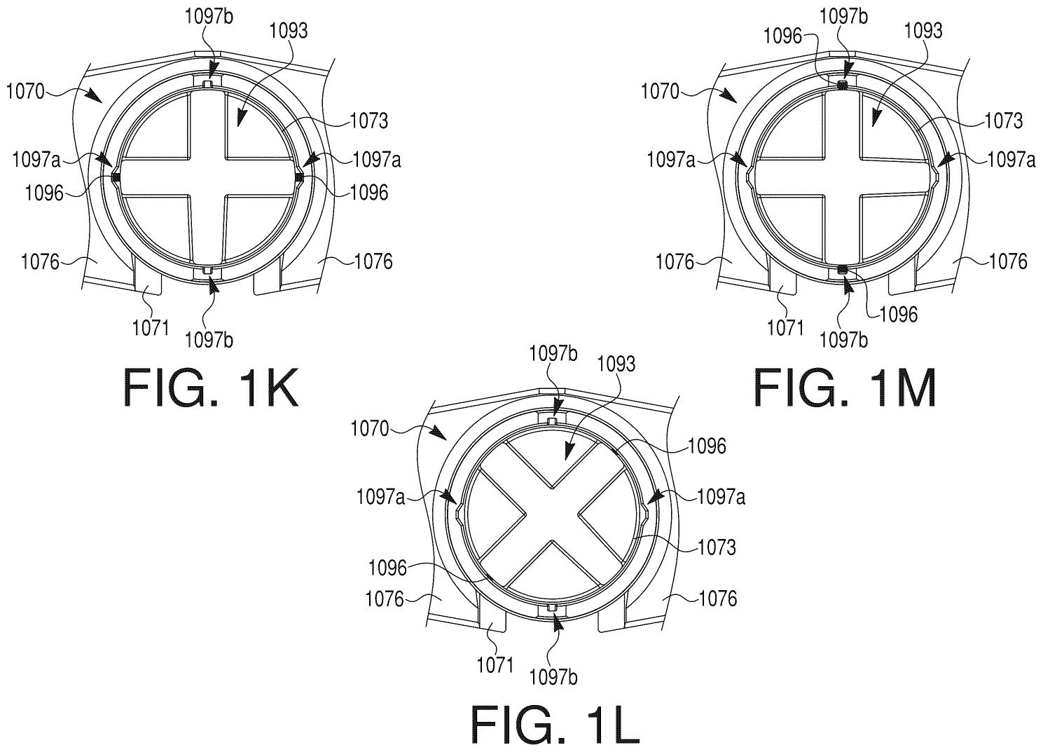

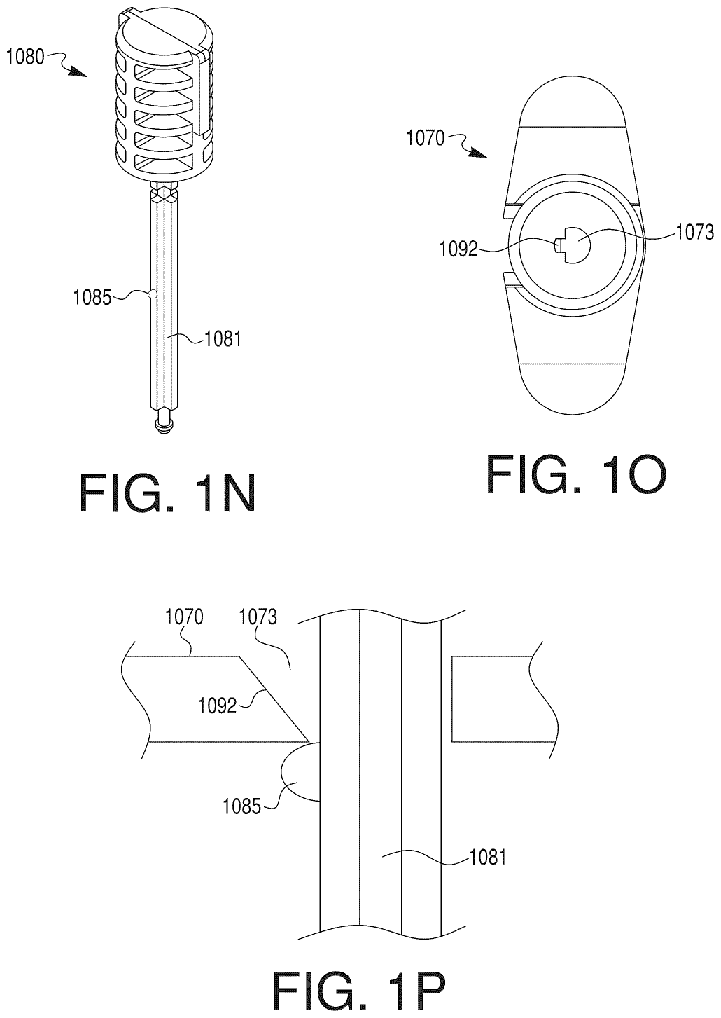

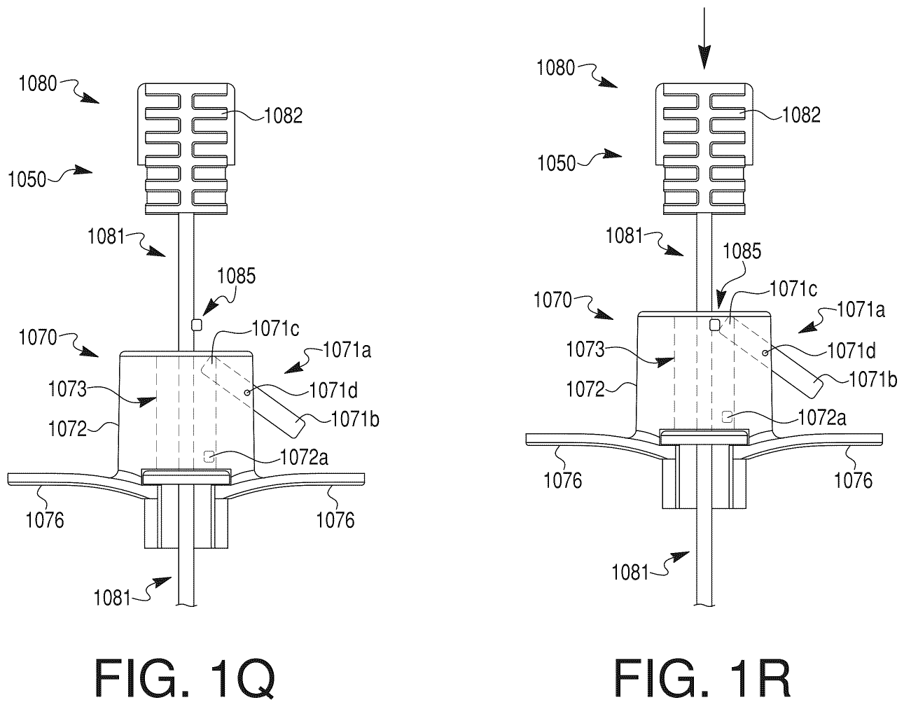

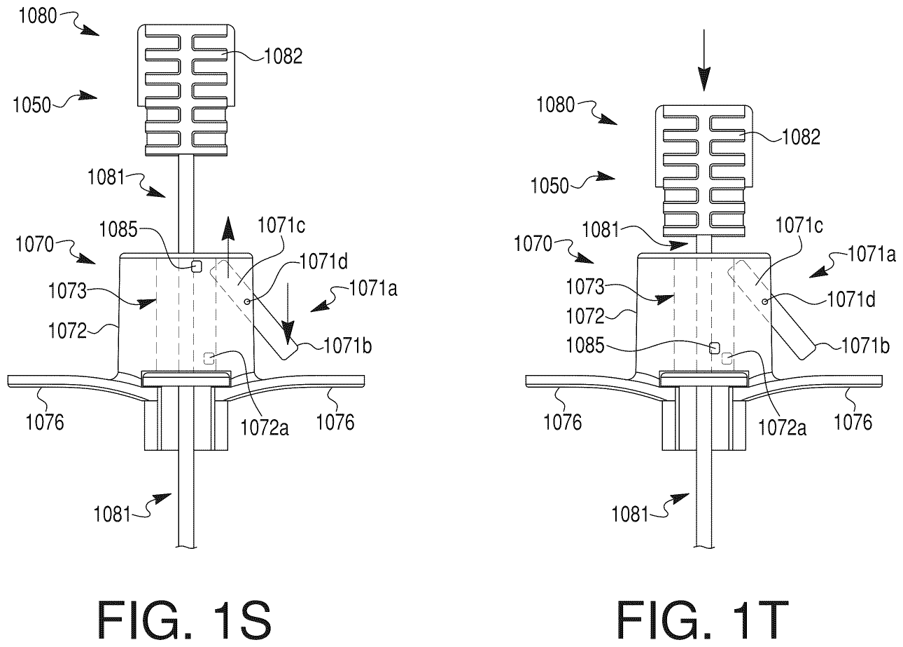

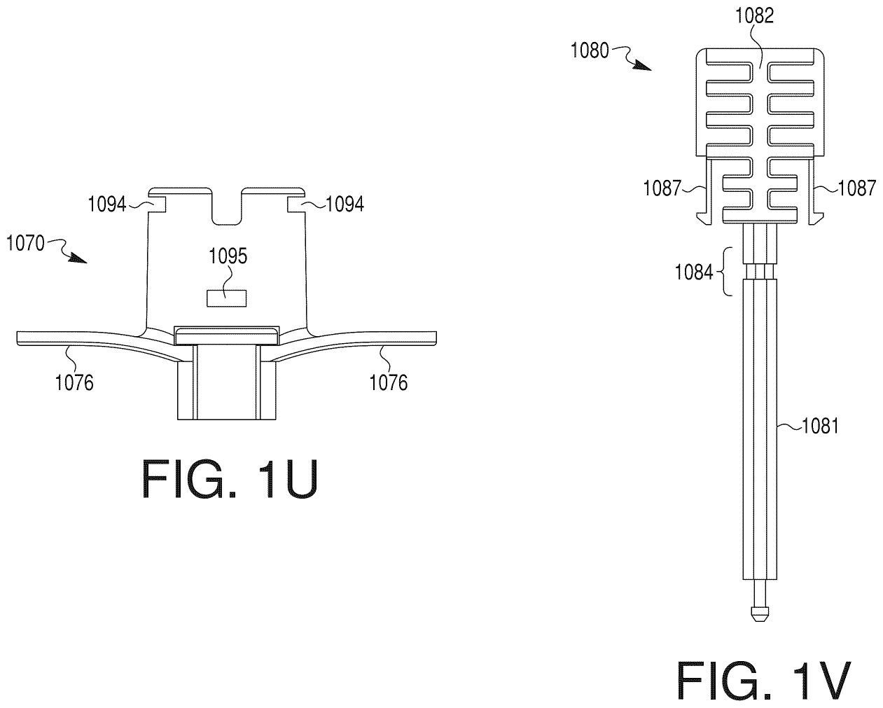

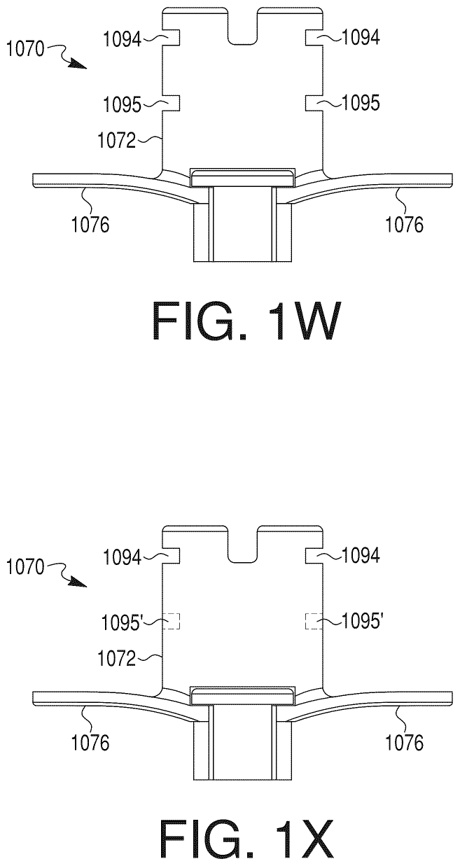

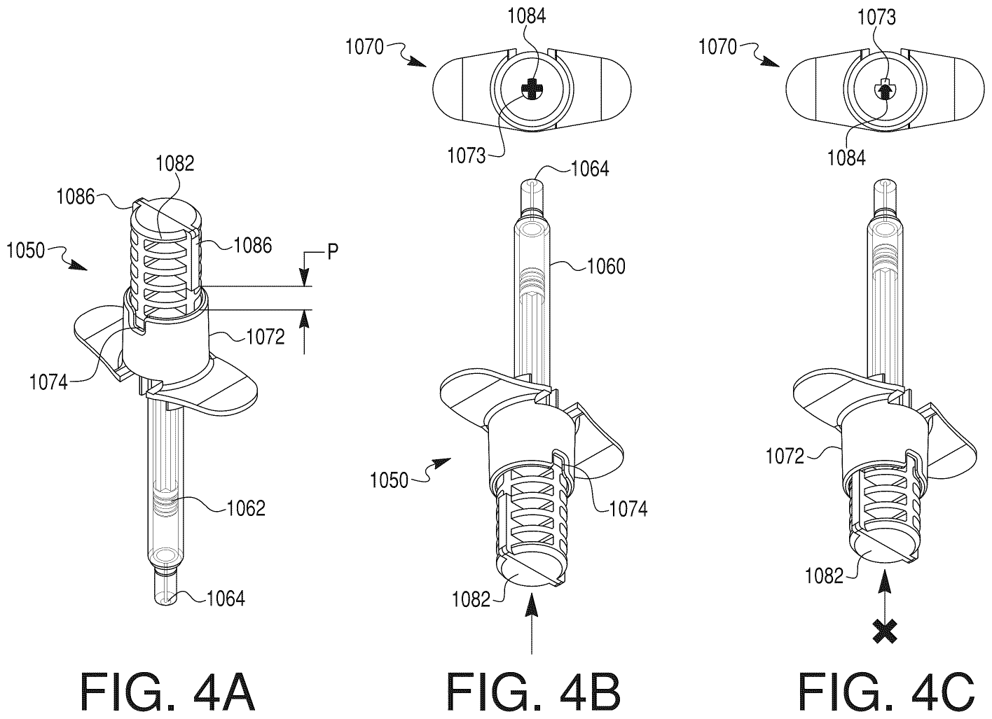

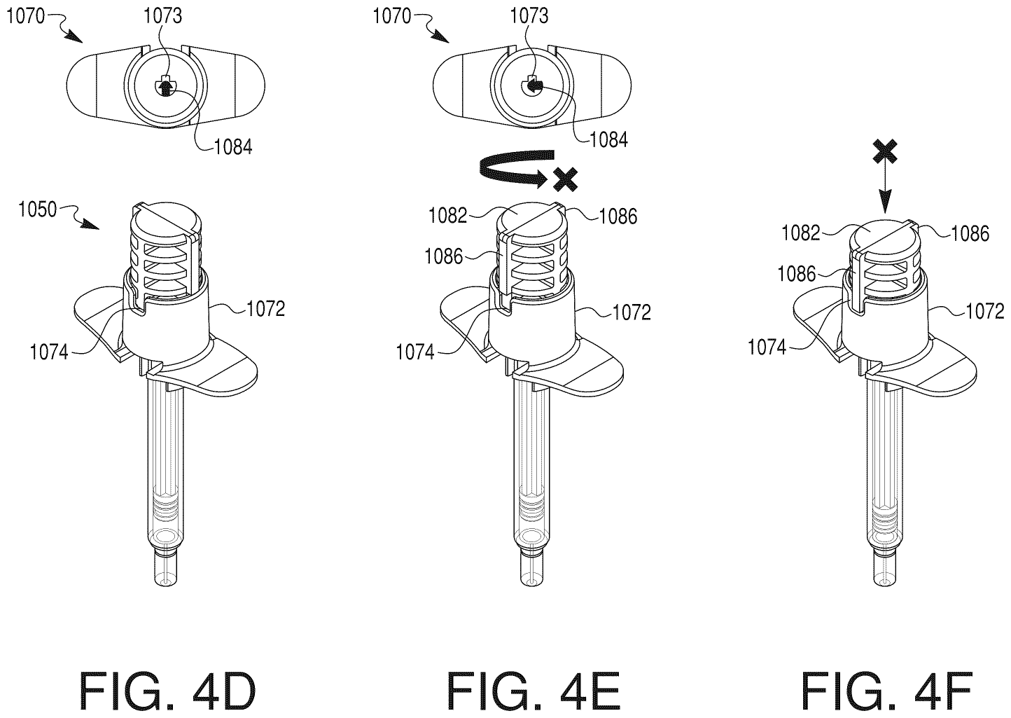

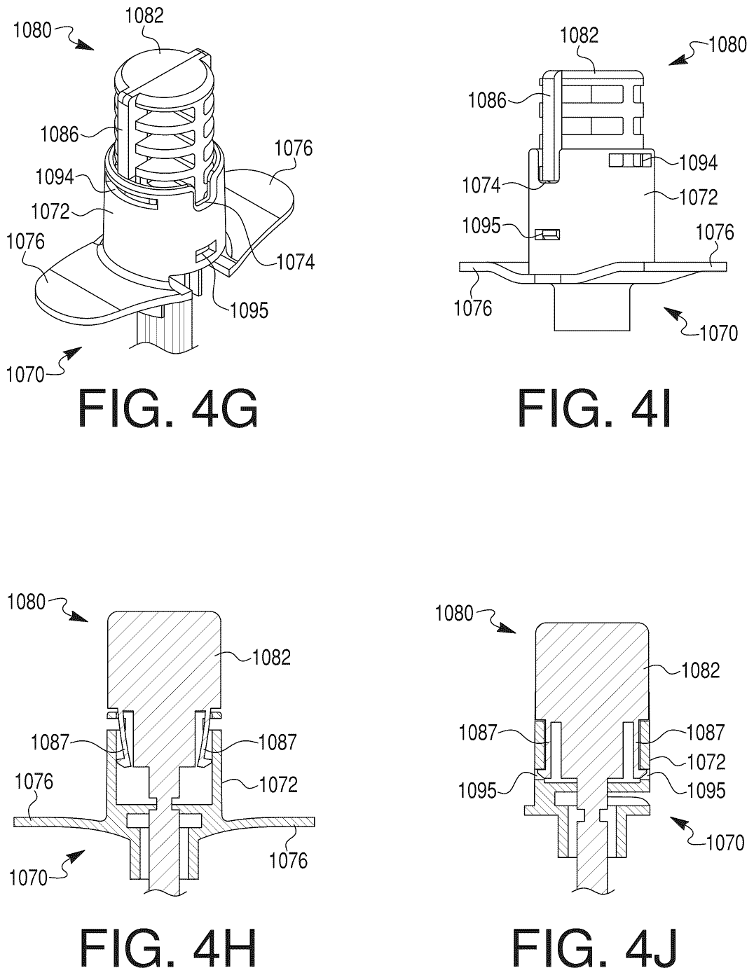

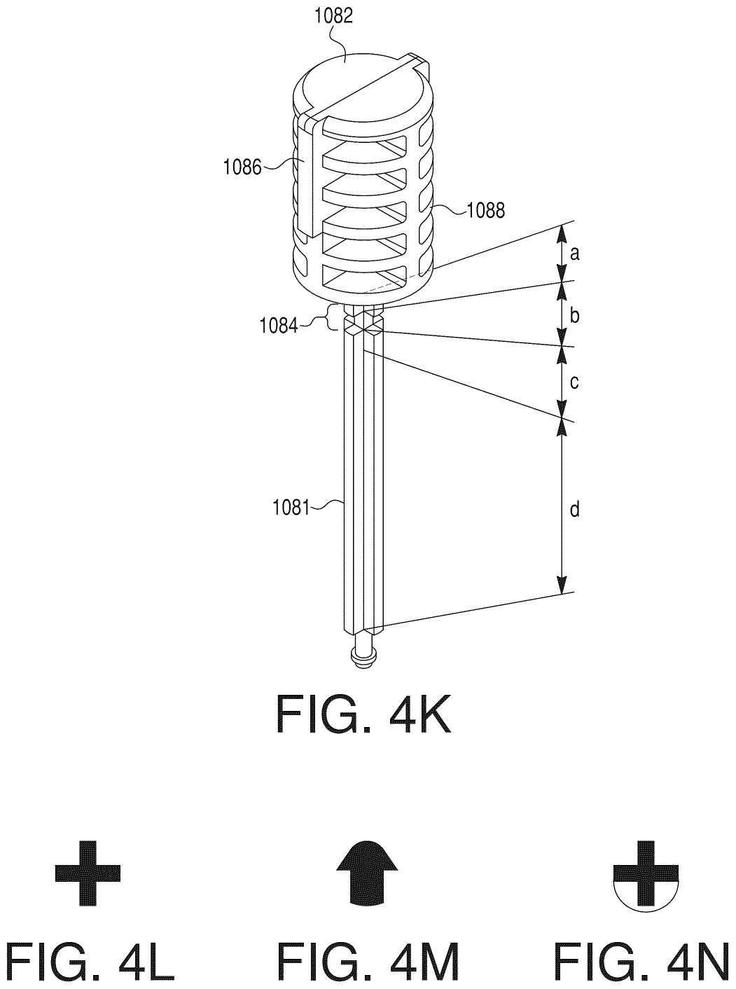

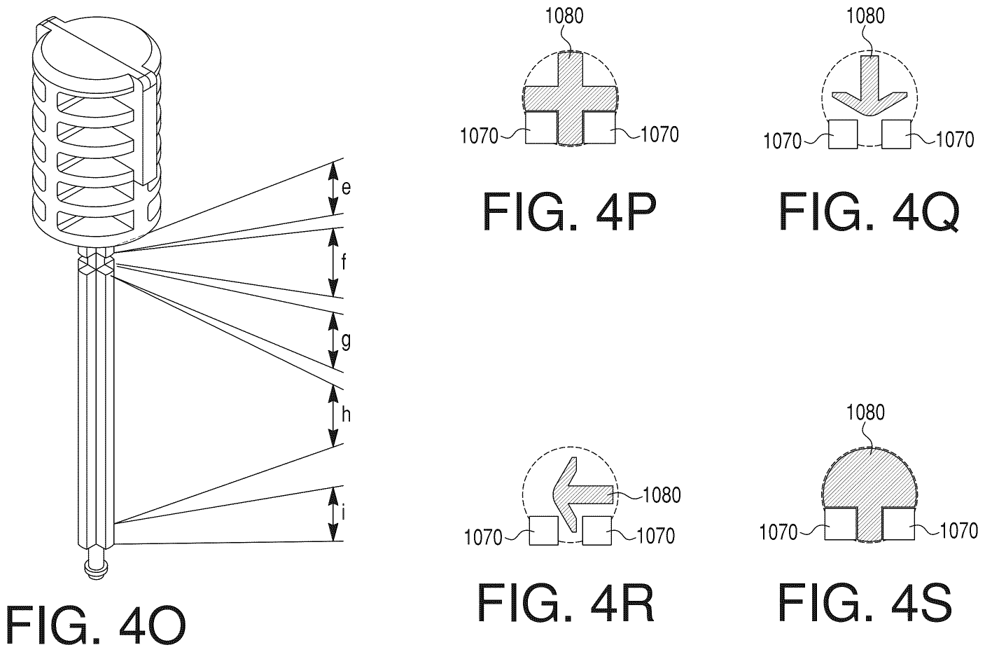

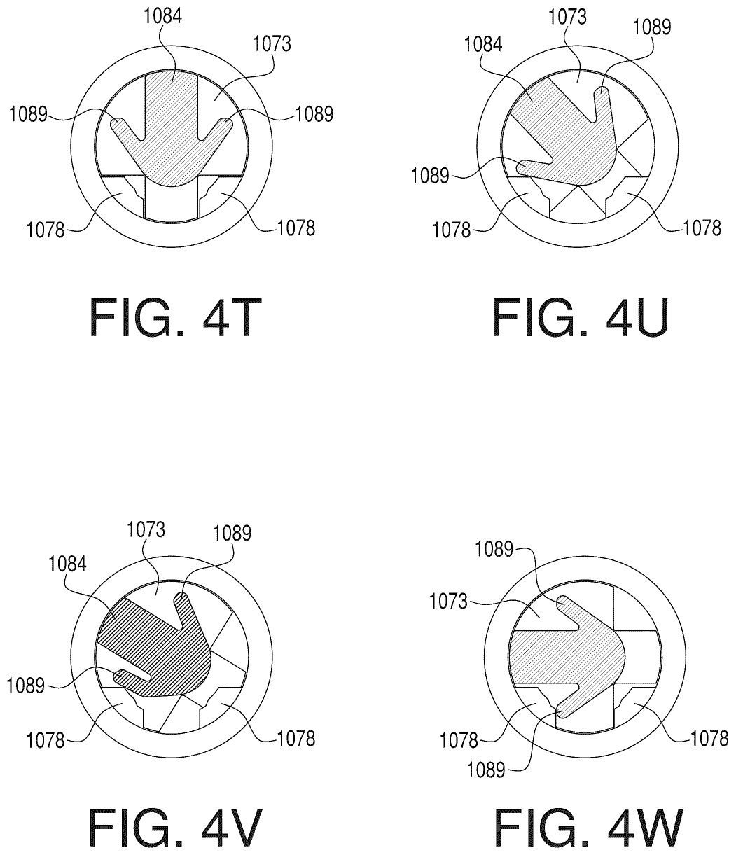

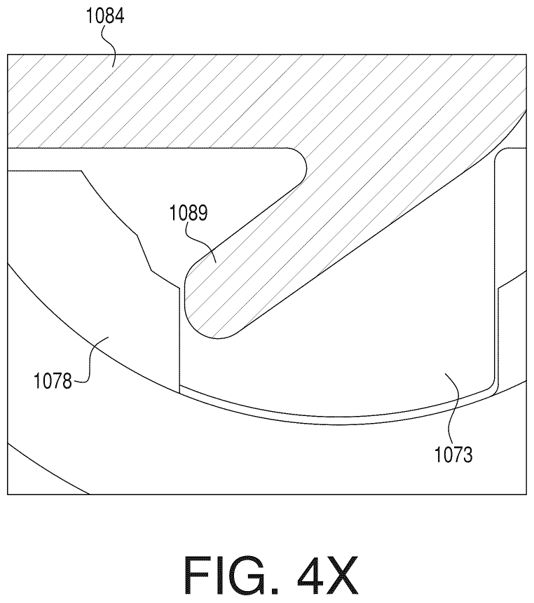

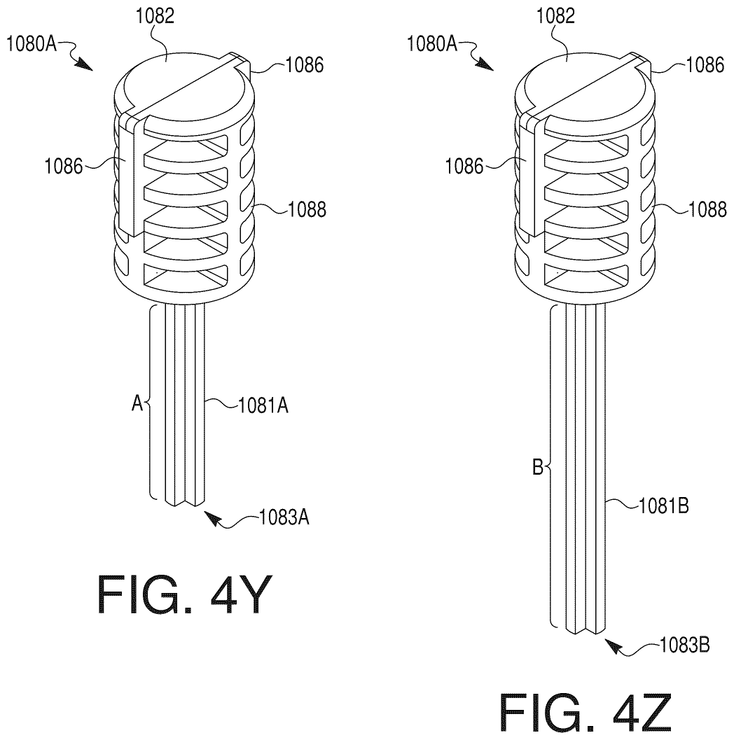

Referring now to FIGS. 1A-1E, views of a delivery device 1050 and component parts are depicted. Device 1050 includes a body 1060, and a blocking component in the form of a flange piece 1070 with a proximal collar 1072 surrounding an opening 1073 (shown in, e.g., FIGS. 4B-4E), through which a plunger rod 1080 may pass into body 1060. Plunger rod 1080 includes an actuation portion 1082 which may be actuated (e.g., pushed or twisted) to rotate plunger rod 1080, or to move plunger rod 1080 longitudinally into body 1060. Actuation portion 1082 may be sized and configured to fit (e.g., nest or otherwise fit) inside proximal collar 1072.

Device 1050 may be, for example, an injection device, such as a syringe, for dispensing a predetermined volume of a formulated drug substance. In some embodiments, device 1050 may be a pre-filled syringe. For example, a user may receive an assembled and packaged device 1050 ready for use, with a volume of formulated drug substance already disposed between a stopper 1062 in body 1060 and an expulsion end 1064 of body 1060. In some embodiments, an air bubble (not shown) may also be disposed between stopper 1062 and expulsion end 1064. In further embodiments, device 1050 may be a fillable syringe.

Body 1060 may be any suitable body configured for holding and expelling a predetermined volume of a formulated drug substance. In some embodiments, body 1060 may have, e.g., a hollow cylindrical portion. Body 1060 may be configured to hold any suitable volume of a formulated drug substance for delivering to, e.g., a patient, and (together with other components of device 1050) to expel a predetermined amount of the held volume through, e.g., expulsion end 1064 in a priming step and/or delivery step. In some embodiments, body 1060 may be configured to hold and (together with other components of device 1050) expel a relatively small volume of formulated drug substance (e.g., less than about 100 μl, such as less than about 80 μl, less than about 60 μl, less than about 40 μl, less than about 20 μl, less than about 10 μl, about 95 μl, about 90 μl, about 85 μl, about 80 μl, about 75 μl, about 70 μl, about 65 μl, about 60 μl, about 55 μl, about 50 μl, about 45 μl, about 40 μl, about 35 μl, about 30 μl, about 25 μl, about 20 μl, about 15 μl, about 10 μl, or about 5 l). Device 1050, together with its other components, may be further configured to minimize a residual volume of the formulated drug substance remaining in body 1060 after delivering the predetermined small volume to the patient. In some embodiments, body 1060 may be pre-filled (e.g., prior to completed assembly, packaging, sterilization and/or shipment of device 1050 to users). In some embodiments, stopper 1062 may be configured to hold a predetermined volume of a formulated drug substance inside a cavity of body 1060.

Flange piece 1070 may be of any suitable size and/or shape to serve as a blocking component in delivery device 1050, to close, partially close, cover, or partially cover an end of body 1060 opposite expulsion end 1064, and/or to support and hold plunger rod 1080 in place inside body 1060. In some embodiments, flange piece 1070 may include a distal collar 1075 configured to engage with body 1060 and hold flange piece 1070 in place in relation to body 1060. For example, distal collar 1075 may include a lip 1071 that may slide under or otherwise in relation to a body flange 1061, to hold flange piece 1070 in place (e.g., to slidably couple flange piece 1070 to body 1060). In alternative embodiments, lip 1071 of distal collar 1075 may be made of a flexible or semi-flexible material, so that it may snap in place over body flange 1061. In further embodiments, distal collar 1075 or another portion of flange piece 1070 may be adhered to, molded to, or otherwise affixed to, body 1060, or may engage with body 1060 via a friction fit.

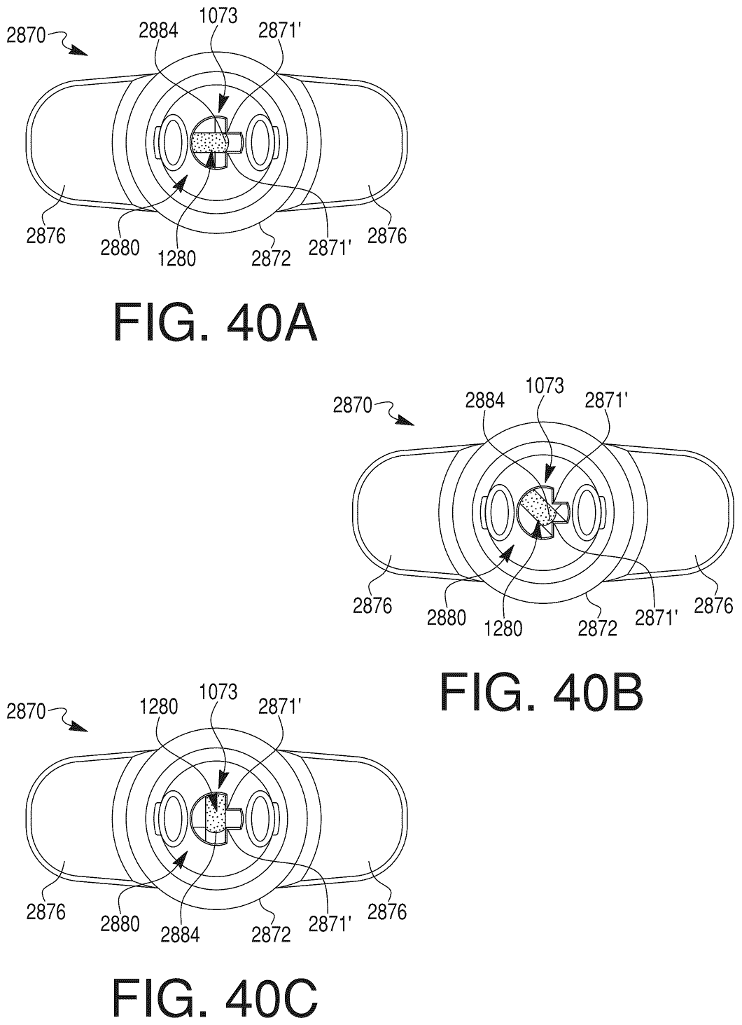

Flange piece 1070 may be or include a blocking component; i.e., part or all of flange piece 1070 may be sized and configured to control movement of plunger rod 1080 by blocking movement of plunger rod 1080 when plunger rod 1080 is in certain configurations relative to flange piece 1070. For example, flange piece 1070 may be configured to control rotational and longitudinal movement of plunger rod 1080, e.g., via opening 1073 (see, e.g., FIGS. 4B-4E) that complements the size and shape of parts of plunger rod 1080 (e.g., neck 1084 and actuation portion 1082, and/or other portions of plunger rod 1080 as shown in FIGS. 4K and 40). As described in further detail herein, flange piece 1070 may be formed of various materials having a minimum strength and/or rigidity which may provide further control of a rotational or longitudinal movement of plunger rod 1080. For example, flange piece 1070 may be configured to resist proximal movement (or “pull back”) of plunger rod 1080 (e.g., to inhibit disassembly of device 1050 by retracting plunger rod 1080) up to a predetermined force based at least in part on a material composition of flange piece 1070. It should be appreciated that flange piece 1070 may be configured such that applying a force exceeding the predetermined force may cause one or more of flange piece 1070 and plunger rod 1080 to break, thereby rendering device 1050 inoperable.

By way of further example, flange piece 1070 may be configured to resist rotational movement of plunger rod 1080 (e.g., to inhibit inadvertent rotation) up to a predetermined force based at least in part on a material composition of flange piece 1070. Additionally and/or alternatively, flange piece 1070 may be configured to resist distal movement of plunger rod 1080 to control a rate of dosage delivery (e.g., to inhibit inadvertent delivery) based at least in part on a material composition of flange piece 1070. Various other components of device 1050 other than flange piece 1070 may include a material composition providing a frictional interference to inhibit disassembly of device 1050, inadvertent rotation of plunger rod 1080, and/or inadvertent dosage delivery.

Proximal collar 1072 of flange piece 1070 may be sized and configured to accept part of actuation portion 1082 of plunger rod 1080, while blocking protrusions 1086 of plunger rod 1080 from moving distally past a predetermined point until plunger rod 1080 is rotated to a particular position. As shown in FIGS. 1A and 1B, collar 1072 may be cylindrical; in alternate embodiments, collar 1072 may have any suitable size or shape compatible with actuation portion 1082. Collar 1072 may also include cavities, e.g., slots 1074 into which protrusions 1086 of plunger rod 1080 may be received. Slots 1074 may have proximally-facing openings and may have a depth dimension parallel to a longitudinal axis of device 1050. A number and configuration of slots 1074 may correspond to a number and configuration of protrusions 1086 on plunger rod 1080. In some embodiments, slots 1074 may be disposed about a perimeter of collar 1072 in a radially symmetrical configuration. In further embodiments, collar 1072 may include only one slot 1074. The depth of slots 1074 may correspond to a distance plunger rod 1080 must move in order to push stopper 1062 towards expulsion end 1064, and dispense a predetermined volume of formulated drug substance from body 1060 through expulsion end 1064. Advantageously, the predetermined volume of formulated drug substance that is to be dispensed from body 1060 may be controlled during manufacturing, by, e.g., selecting a particular depth of slots 1074. In some embodiments, device 1050 may be configured such that normal variations in manufacturing of other parts of device 1050 (e.g., body 1060 or plunger rod 1080) may not cause variations in the volume of formulated drug substance that is to be dispensed from body 1060. As such, the predetermined volume may be controlled by simply varying manufacture of flange piece 1070.

In some embodiments, flange piece 1070 may include one or more flanges 1076, which may be sized and configured to aid a user in holding device 1050 and/or expelling a formulated drug substance from device 1050. In some embodiments, as depicted in FIGS. 1A-1E, flange piece 1070 may include two flanges 1076 opposite to one another and extending perpendicularly from a longitudinal dimension of device 1050. In some embodiments, flange piece 1070 may include other arrangements of a flange or flanges, such as four flanges, or one circumferential flange extending radially outward from a central longitudinal axis of device 1050. In some embodiments, flange piece 1070 may extend radially outward from a central longitudinal axis of device 1050 farther than a circumference of body 1060. In such embodiments, flange piece 1070 may support device 1050 if device 1050 is placed on a surface, may prevent device 1050 from rolling on a flat surface, and/or may allow device 1050 to be picked up more easily. In still further embodiments, blocking component aspects of flange piece 1070 (e.g., collar 1072) may be separate from flange piece 1070, such that delivery device 1050 includes a separate flange piece and blocking component.

Plunger rod 1080 in general may be rotatable about a central longitudinal axis (e.g., in one direction or in both directions). In some embodiments, rotation of plunger rod 1080 may be accomplished by grasping and/or twisting actuation portion 1082 relative to flange piece 1070 and/or body 1060. In some embodiments, protrusions 1086 may assist a user in grasping and/or twisting actuation portion 1082 relative to flange piece 1070 and/or body 1060, by providing additional surface area that a user may grasp and/or push against to twist actuation portion 1082. In some embodiments, only a part or parts of plunger rod 1080 (e.g., actuation portion 1082 and/or a neck 1084) may be rotatable relative to flange piece 1070 and/or body 1060. In some embodiments, plunger rod 1080 may be configured to rotate relative to flange piece 1070 in response to applying a predetermined twisting force onto actuation portion 1082. A material composition of flange piece 1070 may be determinative of the predetermined twisting force required to rotate plunger rod 1080 relative to flange piece 1070. For example, flange piece 1070 may be formed of various materials having a predetermined rigidity that may generate frictional resistance against plunger rod 1080 to control rotational movement of plunger rod 1080 up to the predetermined force (e.g., to inhibit inadvertent rotation/accidental twisting of plunger rod 1080). Further, a material composition of flange piece 1070 may provide a frictional tolerance to control a distal translation of plunger rod 1080 up to a predetermined force (e.g., to inhibit inadvertent dosage delivery by device 1050).

A stem 1081 of plunger rod 1080 may have any thickness and cross-sectional shape suitable for fitting into body 1060, while maintaining sturdiness. For example, in some embodiments, stem 1081 may have as great a thickness, along at least one dimension, as can fit and slide into body 1060. Advantageously, such a thickness may help in preventing unwanted wobbling of plunger rod 1080 relative to the other components of device 1050. In further embodiments, stem 1081 may have a smaller thickness while still maintaining sturdiness (e.g., not bending, breaking, or warping during assembly and/or use of device 1050). In some embodiments, portions of stem 1081 may be configured to allow for plunger rod 1080 to rotate relative to flange piece 1070, whereas other portions of stem 1081 may not (see, e.g., FIGS. 4K-4S).

Plunger rod 1080 may also include a distal tip 1083 (see, e.g., FIG. 1D) sized and configured to push, attach to, or otherwise interface with stopper 1062. Tip 1083 may have any size or shape suitable to achieve this purpose. In some embodiments, for example, tip 1083 may be sized and configured to clip to stopper 1062 via an opening in stopper 1062. In further embodiments, tip 1083 may have a ball-shape configured to fit into an opening in stopper 1062. In yet further embodiments, tip 1083 may present a flat surface parallel to a proximal surface of stopper 1062, and may be configured to push stopper 1062 distally without attaching to stopper 1062. In further embodiments, tip 1083 may have any shaped surface suitable for pushing stopper 1062 distally.

In some embodiments, neck 1084 of plunger rod 1080 and opening 1073 of flange piece 1070 may have complementary geometries that restrict the extent and direction that plunger rod 1080 (or a part thereof) may rotate, depending on the specific longitudinal and/or rotational position of plunger rod 1080 relative to flange piece 1070. In some embodiments, actuation portion 1082 of plunger rod 1080 and collar 1072 may also include complementary geometries that control the extent and direction that plunger rod 1080 may move relative to flange piece 1070. For example, rotation and/or longitudinal movement of plunger rod 1080 may be restricted based on priming, preparing, and/or drug delivery steps of a method of using device 1050 (see, e.g., the method described with respect to FIGS. 4A-4F and the additional/alternative method described with respect to FIGS. 4G-4H and 4I-4J), and the corresponding position of plunger rod 1080 with respect to each step in such methods. For example, plunger rod 1080 may be restricted from being moved out of flange piece 1070 in a proximal direction (e.g., falling out or being pulled out) once device 1050 is assembled. Moreover, plunger rod 1080 may be restricted from rotation about a longitudinal axis before device 1050 is in a “primed” state, and/or after device 1050 is in a “delivery” state. Additionally, longitudinal movement of plunger rod 1080 in the proximal direction (e.g., to “back out” plunger rod 1080), may be restricted after device 1050 is in a “primed” and/or “delivery” state by complementary geometries of neck 1084 of plunger rod 1080 and opening 1073 of flange piece 1070 and/or of actuation portion 1082 of plunger rod 1080 and collar 1072 of flange piece 1070. Advantageously, this may prevent unwanted plunger rod back out in cases where plunger rod 1080 is not held inside body 1060 by, e.g., being affixed to stopper 1062. For example, in some embodiments, plunger rod 1080 may be configured to simply contact or rest against stopper 1062, such that proximal movement of plunger rod 1080 does not move stopper 1062 proximally. In such cases, proximal movement of plunger rod 1080 may be prevented by interaction between complementary geometries of plunger rod 1080 and flange piece 1070. Moreover, interaction between actuation portion 1082 of plunger rod 1080 and collar 1072 of flange piece 1070 may restrict longitudinal movement of plunger rod 1080 in a distal direction. As an example, plunger rod 1080 may be restricted from moving distally after the “primed” state but before the “delivery” state.

Upon being moved to the “delivery” state, protrusions 1086 on actuation portion 1082 may be longitudinally aligned with slots 1074 of collar 1072, allowing for distal movement of plunger rod 1080 to dispense a desired volume of a drug substance from body 1060. As such, plunger rod 1080 may include a number and configuration of protrusions 1086 such that each protrusion 1086 may move distally into a slot 1074 when plunger rod 1080 is in a particular position (e.g., a “delivery” state). In some embodiments, one, two, three, or more protrusions 1086 may extend from actuation portion 1082, corresponding to one, two, three, or more slots 1074, respectively. For example, as depicted, two protrusions 1086 may extend from the sides of actuation portion 1082 in a radially symmetrical configuration (corresponding to two slots 1074 in collar 1072). In some embodiments, radial symmetry of multiple protrusions 1086 (and slots 1074) may advantageously allow for protrusions 1086 to fit into slots 1074 in multiple configurations (e.g., depending on whether actuation portion 1082 is twisted in one direction or another). In such embodiments, actuation portion 1082 may be twisted in either direction based on, e.g., user preference, right-handedness or left-handedness, or other factors. In some embodiments, plunger rod 1080 may not be pulled proximally or backed out of body 1060 (e.g., towards actuation portion 1082) after plunger rod 1080 is in a “primed” state and/or after a desired volume of formulated drug substance has been delivered from device 1050 by depression of plunger rod 1080 into body 1060 (e.g., due to a geometry of neck 1084 and/or opening 1073).

In some embodiments, device 1050 may be configured for ease of use, and may include one or more features that aid a user by providing tactile or visual feedback. For example, one, two, or more components of device 1050 may have contrasting colors or textures. In some embodiments, for example, flange piece 1070 may have a different coloring than plunger rod 1080. As a further example, a single component of device 1050 may have two or more colors or textures. In some embodiments, for example, actuation portion 1082 may include a first color on a distal part of actuation portion 1082, that becomes covered by collar 1072 when device 1050 is primed, and a second color on a second portion of actuation portion 1082, that moves adjacent to collar 1072 when device 1050 is primed, to help indicate to a user that device 1050 has been properly primed. As a further example, in some embodiments, flange piece 1070 may have a different tactile feel than plunger rod 1080 and/or body 1060. For example, flange piece 1070 may be relatively rougher or smoother than plunger rod 1080 and/or body 1060. As yet another example, one or more components of device 1050 may have textures that aid in holding, gripping, identifying, or using device 1050. For example, flange piece 1070 may have a slightly rough or raised texture to aid a user in gripping flanges 1076, and/or to prevent a user's fingers from slipping off of the flanges 1076 during use. In some embodiments, some or all of flange piece 1070 may have a smooth-feeling surface. As another example, actuation portion 1082 of plunger rod 1080 may include a rough or raised texture to aid in gripping and rotating plunger rod 1080. For example, as depicted in FIGS. 1A-1I, 3A-3C, 3E, and 4A-4I, actuation portion 1082 may include circumferential ribbing on its side(s). Actuation portion 1082 may have any suitable number of ribs on its side(s) to provide texture. In further embodiments, actuation portion 1082 may have no ribbing on its side(s).

In some embodiments, device 1050 or one or more of its components may include colors, labels or markers, which may indicate contents or a status of device 1050, and/or which may direct or provide instructions to a user of device 1050. Examples include one or more labels to indicate a priming position versus a dosage delivery position of the plunger rod, one or more labels to indicate directions in which to rotate or otherwise move plunger rod 1080, and/or one or more labels to indicate an amount of formulated drug substance included in device 1050 (e.g., linear markings on body 1060). Labels may be, e.g., adhered or printed on components of device 1050, or may be embossed on, or molded as a part of, components of device 1050. In some embodiments, one or more textured labels (e.g., embossed or molded on device 1050) may also serve as a textured, rough, or raised surface to aid a user in gripping or using device 1050. One or more exemplary labels may include words, numerals, indicators, and/or symbols (e.g., lines, padlocks, arrows, diagrams, etc.).

In some embodiments, device 1050 may be configured to make one or more sounds and/or include haptics, such as audible cues, during its use. For example, device 1050 may make a “clicking” noise upon completion of a priming step, or upon rotation of the plunger rod to a position suitable for dispensing a predetermined volume of a formulated drug substance. A “clicking” noise may be produced by, e.g., friction between two or more components (e.g., plunger rod 1080 and flange piece 1070), or a portion of one component contacting another portion (e.g., neck 1084 of plunger rod 1080 contacting opening 1073 of flange piece 1070). In some embodiments, device 1050 may include one or more detents or protrusions on adjacent surfaces of, e.g., plunger rod 1080 and flange piece 1070, which may produce a clicking sound when contacting one another (e.g., wings 1089 on neck 1084 contacting detents 1078 surrounding opening 1073, as shown in FIGS. 4T-4X). Such sounds may serve as auditory feedback to indicate that a user has reached a particular step in the use of device 1050.

In some embodiments, device 1050 may include additional features or components to control movement of plunger rod 1080 relative to body 1060. For example, as shown in FIG. 1F, flange piece 1070 may include an opening 1079 through which a pin 1077 may be disposed. Pin 1077 may be sized and configured to interface with actuation portion 1082 of plunger rod 1080 (e.g., to slide into an opening (not shown) in actuation portion 1082), such that when pin 1077 is inserted so as to engage actuation portion 1082, plunger rod 1080 may not be moved proximally or distally relative to body 1060 and flange piece 1070. In some embodiments, pin 1077 may also prevent rotational movement of plunger rod 1080 relative to flange piece 1070. Pin 1077 may be inserted upon filling and assembly of a device (e.g., device 1050 shown in FIGS. 1A and 1B), to prevent unwanted movement of plunger rod 1080 prior to its use. In some embodiments, pin 1077 may remain inserted during packaging, transportation, and delivery of device 1050. Before use of device 1050, pin 1077 may be removed or otherwise positioned so that it does not engage actuation portion 1082.

As shown in FIGS. 1G and 1H, in some embodiments, a protrusion 1093 may be disposed at a distal portion of actuation portion 1082, which may be located inside flange piece 1070 upon assembly of device 1050. An inward lip 1091 of flange piece 1070 may overhang protrusion 1093, such that actuation portion 1082 may not be pulled proximally out of flange piece 1070. In some embodiments, either protrusion 1093, lip 1091, or both may be disposed circumferentially about actuation portion 1082, such that lip 1091 blocks protrusion 1093 regardless of a rotational position of actuation portion 1082 relative to flange piece 1070. Protrusion 1093 and lip 1091 may have squared-off cross-sectional profiles, as shown in FIG. 1G, angled cross-sectional profiles, as shown in FIG. 1H, or any other suitable cross-sectional profiles. In some embodiments, a cross-sectional profile of protrusion 1093, lip 1091, or both may be selected to improve ease of manufacturing (e.g., machining or molding the shape of protrusion 1093 or lip 1091), or may be selected to improve assembly (e.g., insertion of plunger rod 1080 into and partially through flange piece 1070).

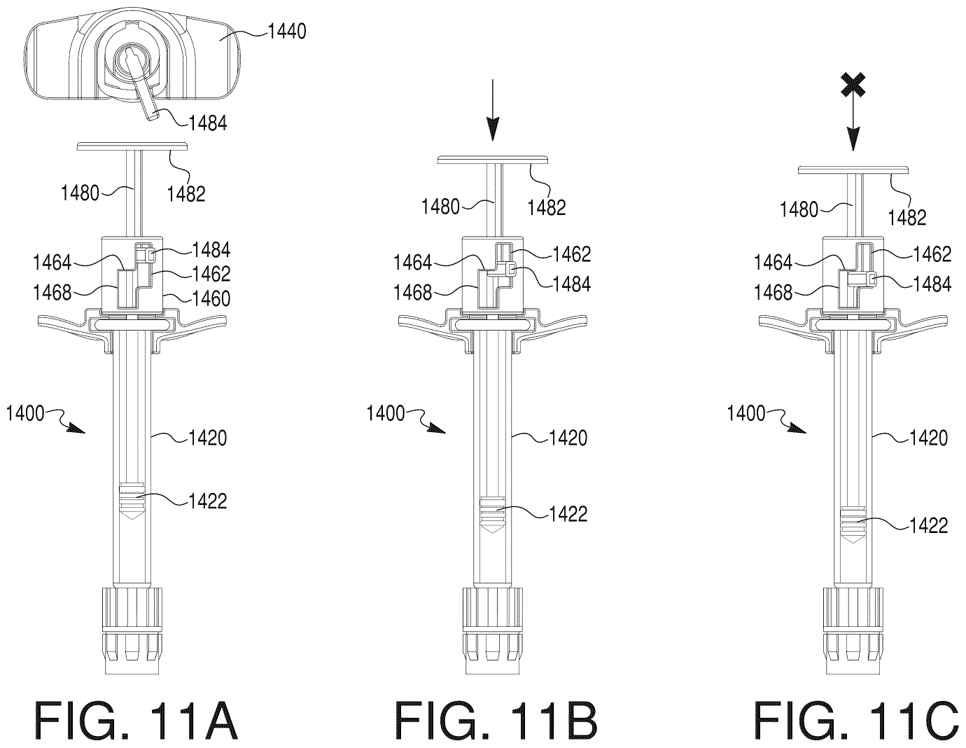

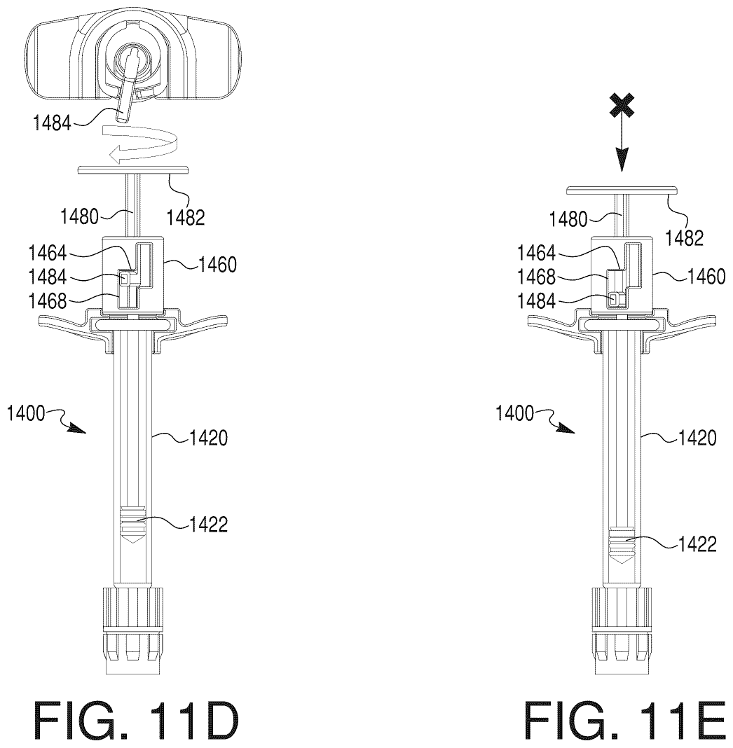

As shown in FIG. 11, in some embodiments, actuation portion 1082 may include one or more projections 1096 extending radially outward from an exterior perimeter of protrusion 1093. For example, protrusion 1093 may include a pair of projections 1096 disposed about protrusion 1093 at opposite locations relative to one another. Projections 1096 may include various suitable sizes, shapes, and/or cross-sectional profiles. In some embodiments, projections 1096 may have a circular shape with a rounded exterior profile to facilitate movement of protrusion 1093 within flange piece 1070.

In the present example, projections 1096 may be positioned along a side of protrusion 1093 that longitudinally aligned with a corresponding side of actuation portion 1082 including protrusions 1086. In other examples, projections 1096 may be positioned along a side of protrusion 1093 that is offset (e.g., not in longitudinal alignment) with the side of actuation portion 1082 including protrusions 1086. Projections 1096 may be formed of various flexible materials, including, for example, a polymer such as plastic, rubber, etc. It should be appreciated that plunger rod 1080 may include additional and/or fewer projections 1096 on protrusion 1093, or other portions of actuation portion 1082, than those shown and described herein without departing from this disclosure.

FIG. 1J depicts a distal end portion of flange piece 1070 including one or more recesses 1097 along an interior surface. Recesses 1097 may be sized and shaped to receive projections 1096 when protrusion 1093 is received within flange piece 1070 and positioned adjacent and/or in contact with lip 1091. It should be appreciated that lip 1091 may be configured to require application of a hydrodynamic force onto plunger rod 1080 to receive projections 1096 and protrusion 1093 distally of lip 1091 and into flange piece 1070, thereby priming device 1050 and inhibiting retraction (e.g., proximal movement) of plunger rod 1080 relative to flange piece 1070. It should be appreciated that by inhibiting removal of plunger rod 1080 after an initial assembly into flange piece 1070, device 1050 may be configured to prevent reuse of device 1050 after an initial use, and/or to prevent inadvertent air intake forming bubbles within device 1050. In the present example, flange piece 1070 may include a plurality of recesses 1097 disposed about the distal end portion in an annular array relative to one another. The plurality of recesses 1097 may be spaced apart from one another about a circumference of flange piece 1070. In some embodiments, flange piece 1070 may include recesses 1097 having varying sizes and/or shapes relative to one another.

As described in further detail below, a subset of the plurality of recesses 1097 may be sized and shaped to receive and allow passage of projections 1096 therethrough upon movement of protrusion 1093 relative to flange piece 1070. A second subset of the plurality of recesses 1097 may be sized and shaped to receive and inhibit passage of projections 1096 therethrough such that protrusion 1093 is restricted from further movement relative to flange piece 1070, as explained in further detail below.

For example, as shown in FIG. 1K, flange piece 1070 includes a pair of widened recesses 1097a positioned about opening 1073 (with plunger rod 1080 received therethrough) at opposite locations relative to one another (e.g., spaced about 180 degrees apart from one another). Flange piece 1070 further includes a pair of narrowed recesses 1097b positioned about opening 1073 at opposite locations relative to one another (e.g., about 180 degrees from one another). A recess 1097a may be positioned about 90 degrees apart from an adjacent recess 1097b, along the circumference of flange piece 1070. Widened recesses 1097a may include a center wall transverse (e.g., perpendicular) to a longitudinal length of flanges 1076 and sidewalls that are angled relative to the center wall. Narrowed recesses 1097b may include a center wall parallel to the longitudinal length of flanges 1076 and sidewalls that are perpendicular to the center wall. It should be appreciated that widened recesses 1097a may form a larger opening for receiving projections 1096 relative to narrowed recesses 1097b. It should further be understood that sidewalls of recesses 1097a, 1097b may have a height that is parallel to a longitudinal length of device 1050.

In a first configuration seen in FIG. 1K, plunger rod 1080 is received through flange piece 1070 and protrusion 1093 is oriented relative to opening 1073 such that projections 1096 are received within widened recesses 1097a. The angled sidewalls of widened recesses 1097a may provide clearance to facilitate movement of projections 1096 out of widened recesses 1097a in response to a rotation of plunger rod 1080 relative to flange piece 1070. In this instance, projections 1096 may move along the angled sidewalls of widened recesses 1097a as protrusion 1093 rotates relative to opening 1073.

As seen in FIG. 1L, projections 1096 may abut against the interior surface of flange piece 1070 defining opening 1073 as protrusion 1093 rotates. Projections 1096 may generate a frictional interference against flange piece 1070 while moving between adjacent recesses 1097. FIG. 1M shows protrusion 1093 positioned relative to opening 1073 with projections 1096 aligned with and received in narrowed recesses 1097b. In this instance, plunger rod 1080 may be configured to generate an audible and/or tactile feedback in response to narrowed recesses 1097b receiving projections 1096. For example, a “click” or “snap” noise may be generated in response to a release of pressure applied to projections 1096 by the interior surface of flange piece 1070 when projections 1096 are received in narrowed recesses 1097b. Additionally and/or alternatively, an audible feedback may be produced in response to projections 1096 expanding and striking one or more walls defining narrowed recesses 1097b when received therein.

It should be appreciated that a frictional interference between projections 1096 and flange piece 1070 may be removed upon receipt of projections 1096 within narrowed recesses 1097b. The sidewalls of narrowed recesses 1097b may provide a physical restriction that inhibits further movement of projections 1096. In this instance, plunger rod 1080 may be fixed relative to flange piece 1070 such that protrusion 1093 is inhibited from further rotation relative to opening 1073 when projections 1096 are received within narrowed recesses 1097b.

As shown in FIGS. 1N-1P, in some embodiments, plunger rod 1080 may additionally or alternatively include a protrusion 1085 on stem 1081, which may be configured to interact with opening 1073 of flange piece 1070, such that protrusion 1085 may only move distally through opening 1073. A side 1092 of opening 1073 may be angled to allow for distal passage of protrusion 1085, and to block proximal passage of protrusion 1085, as stem 1081 moves through opening 1073. Protrusion 1085 and/or side 1092 may have any suitable shape or configuration to achieve this purpose. In some embodiments, a shape or configuration of protrusion 1085 and/or side 1092 may be selected to improve ease of manufacturing (e.g., machining or molding the shape of protrusion 1085 and/or flange piece 1070).

In other embodiments, flange piece 1070 may include a movable lever 1071a as seen in FIGS. 1Q-1T. Movable lever 1071a may include a first end 1071b extending outwardly from collar 1072 and a second end 1071c disposed within collar 1072. Movable lever 1071a may be movable (e.g., pivotable) about a rotation pin 1071d. Second end 1071c may be positioned within opening 1073 such that movable lever 1071a is configured to interact with protrusion 1085 upon receipt of plunger rod 1080 in flange piece 1070. Referring initially to FIG. 1Q, plunger rod 1080 may be configured to prime device 1050 by translating stem 1081 distally through flange piece 1070 until encountering movable lever 1071a.

As seen in FIG. 1R, second end 1071c may abut against protrusion 1085 when movable lever 1071a is in an obstructing position. Second end 1071c may be configured to inhibit translation of plunger rod 1080 relative to flange piece 1070 when plunger rod 1080 is in a primed position. It should be appreciated that a distance between second end 1071c and protrusion 1085 may define a priming distance for moving plunger rod 1080 to prime device 1050. Movable lever 1071a may be configured to move (e.g., pivot) relative to collar 1072 and about rotation pin 1071d to displace second end 1071c from the obstruction position. The pivoting axis, along which rotation pin 1071d extends, may be substantially perpendicular to the longitudinal axis along which plunger rod 1080 extends.

For example, as seen in FIG. 1S, movable lever 1071a may be actuated in response to moving first end 1071b distally toward flange 1076 and about rotation pin 1071d. In some embodiments, first end 1071b may be actuated in response to receiving a distally-directed force applied thereto by, for example, a user of device 1050. Second end 1071c may be moved in a proximal direction away from flange 1076 and relative to rotation pin 1071d in response to first end 1071b moving distally, thereby causing second end 1071c to disengage protrusion 1085.

Accordingly, as shown in FIG. 1T, movable lever 1071a may allow plunger rod 1080 to translate relative to flange piece 1070 until protrusion 1085 encounters an abutment 1072a positioned at a distal end of opening 1073. Abutment 1072a may cause plunger rod 1080 to settle into a dose completion position of plunger rod 1080 when protrusion 1085 is engaged thereto. It should be appreciated that a distance between second end 1071c and abutment 1072a may define a dosage delivery distance for moving plunger rod 1080 to dispense a controlled volume of substance from device 1050.

As shown in FIGS. 1U and 1V, in some embodiments, actuation portion 1082 of plunger rod 1080 may additionally or alternatively include one or more extensions 1087 configured to interface with side openings 1094, 1095 in collar 1072 of flange piece 1070. Extensions 1087 may extend distally from actuation portion 1082, and may have an angled or rounded distal portion sized and configured to be pushed inward toward a central axis of plunger rod 1080 when actuation portion 1082 is pushed distally into collar 1072. The angled or rounded distal portion of each extension 1087 may include a hook or clip shaped part 1087a. Extensions 1087 may additionally be made of a flexible material, allowing them to be pushed inward into collar 1072 and spring back outwards when no longer being restricted by a side of collar 1072. Side openings 1094, 1095 in collar 1072 may be sized and configured to receive hook or clip shaped part 1087a of an extension 1087, such that once an extension 1087 reaches a side opening 1094 or 1095, a hook or clip shaped part 1087a may spring outward into the side opening 1094 or 1095 and thereafter prevent proximal movement of plunger rod 1080. A number of extensions 1087 may coincide with a number of each of side openings 1094 and side openings 1095, such that each extension 1087 may be received in a corresponding side opening 1094 or 1095 simultaneously as plunger rod 1080 moves distally relative to flange piece 1070.

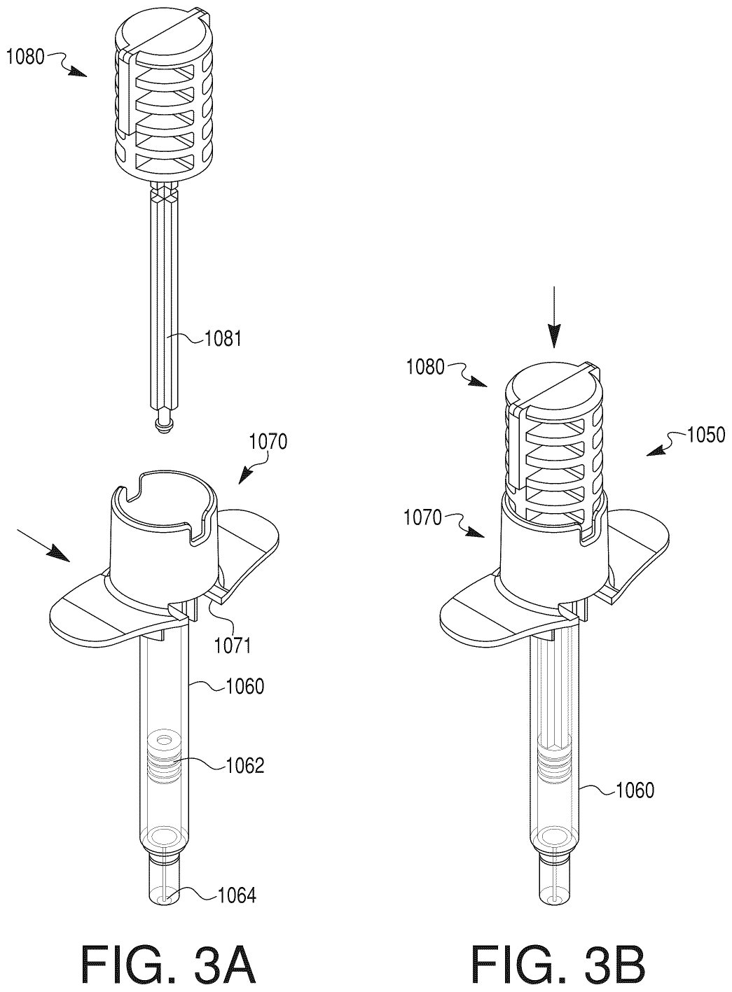

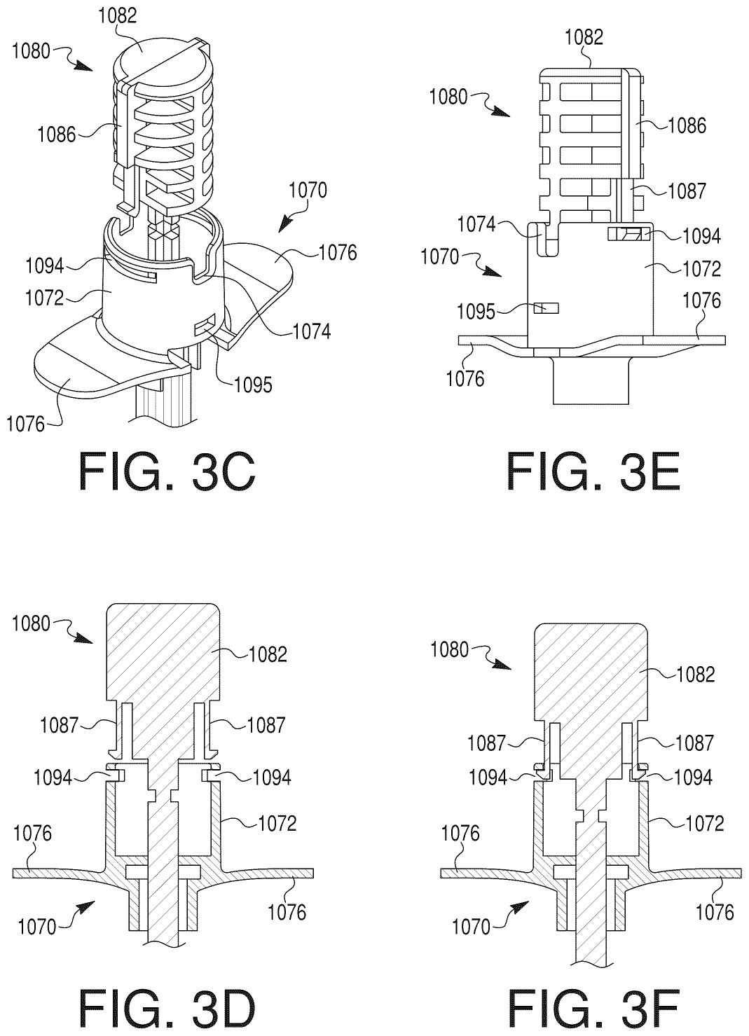

Specifically, first side openings 1094 may be configured to receive hook or clip shaped parts 1087a of extensions 1087 upon assembly of device 1050, to prevent proximal movement of plunger rod 1080 once plunger rod 1080 is inserted to a ready-to-use position. As hook or clip shaped part 1087a of each extension 1087 is received in first side openings 1094, it may make a “clicking” sound as it interfaces with collar 1072, thereby providing auditory and/or tactile feedback, indicating that the device is in a ready-to-use position. In some embodiments, first side openings 1094 may each extend around a partial circumference of collar 1072, such that the hook or clip shaped parts 1087a of extensions 1087 may be received in side openings 1094 in a range of rotational positions of plunger rod 1080 relative to flange piece 1070. Second side openings 1095 may be configured to receive hook or clip shaped parts 1087a of extensions 1087 once device 1050 is in a “delivery” configuration (e.g., after priming and additional rotation of actuation portion 1082 to align protrusions 1086 with slots 1074). In the embodiment depicted in FIGS. 1U and 1V, extensions 1087 are longitudinally aligned with protrusions 1086, and, as depicted in FIGS. 3C-3F, side openings 1095 are likewise longitudinally aligned with slots 1074, to allow for distal movement of actuation portion 1082 further into collar 1072 when device is in the “delivery” configuration. It should be appreciated that device may be transitioned to the “delivery” configuration in response to applying a distally-directed force onto actuation portion 1082, to overcome an engagement of side openings 1094 with extensions 1087, and a rotative force to overcome a frictional force between an interior of collar 1072 and extensions 1087. However, in other embodiments, it is contemplated that extensions 1087 and side openings 1094, 1095 may be in any suitable complementary configuration to assist in controlling proximal movement of plunger rod 1080.

In other embodiments, as shown in FIG. 1W, side openings 1094 may be positioned along collar 1072 in longitudinal alignment with side openings 1095. Device 1050 may be primed upon receiving hook or clip shaped parts 1087a of extensions 1087, initially positioned proximally of side openings 1094, within side openings 1094. In some instances, a feedback (e.g., tactile, auditory, etc.) may be generated in response to extensions 1087 being received within side openings 1094. It should be understood that a proximal end of collar 1072 may resist distal advancement of plunger rod 1080 relative to flange piece 1070 in response to hook or clip shaped parts 1087a being engaged to collar 1072 at side openings 1094. Applying a distally-directed force onto plunger rod 1080 may cause extensions 1087 to be released from side openings 1094 and translated distally through collar 1072 until received within side openings 1095.

It should be appreciated that the distally-directed force required to deflect extensions 1087 inwardly and to release hook or clip shaped parts 1087a from side openings 1094 may correspond to a minimum priming and hydrodynamic force. Accordingly, plunger rod 1080 may be maintained in a constant radial orientation during a priming step and delivery step of device 1050. In other embodiments, additional and/or fewer side openings may be included along a circumferential wall of collar 1072 in longitudinal alignment and/or offset (e.g., not longitudinally aligned) with side openings 1094, 1095.

As seen in FIG. 1X, flange piece 1070 may alternatively include one or more inner projections 1095′ in lieu of side openings 1095 shown and described above. In this instance, plunger rod 1080 may be preassembled into flange piece 1070 with extensions 1087 (FIG. 1V) squeezed into collar 1072 and positioned relatively proximal to side openings 1094. Device 1050 may be primed by pushing plunger rod 1080 distally through flange piece 1070 until extensions 1087 are received within side openings 1094. In some instances, a feedback (e.g., tactile, auditory, etc.) may be generated in response to extensions 1087 being received within side openings 1095. In further embodiments, side openings 1094 may be flared and/or extensions 1087 may have a distally-tapering profile to facilitate further distal advancement of plunger rod 1080 from a primed position to a dose completion position.

Further translation of plunger rod 1080 relative to flange piece 1070 may cause extensions 1087 to bend radially-inward toward one another, thereby allowing plunger rod 1080 to translate distally to deliver a dose from device 1050. Plunger rod 1080 may continue to translate distally relative to collar 1072 until hook or clip shaped parts 1087a (FIG. 1V) encounter inner projections 1095′. Inner projections 1095′ may be configured to contact extensions 1087 and fix plunger rod 1080 to the dose completion position, and/or prevent further distal movement of plunger rod 1080 relative to flange piece 1070. Accordingly, further movement (e.g., proximal and/or distal) of plunger rod 1080 relative to flange piece 1070 may be inhibited by inner projections 1095′ engaging hook or clip shaped parts 1087a within collar 1072. Inner projections 1095′ may include complimentary hooks or clip-shaped parts that are sized and/or shaped to interact with hook or clip shaped parts 1087a or extensions 1087. It should be appreciated that a distance between side openings 1094 and inner projections 1095′ may define a dosage delivery distance to dispense a controlled volume of substance from device 1050.

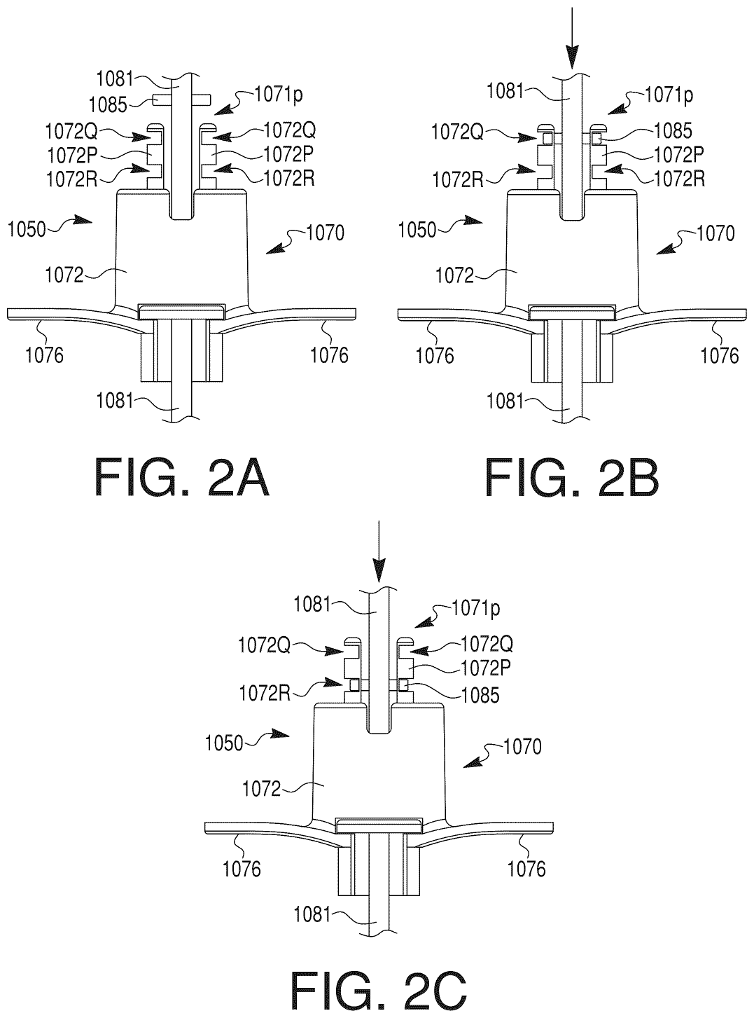

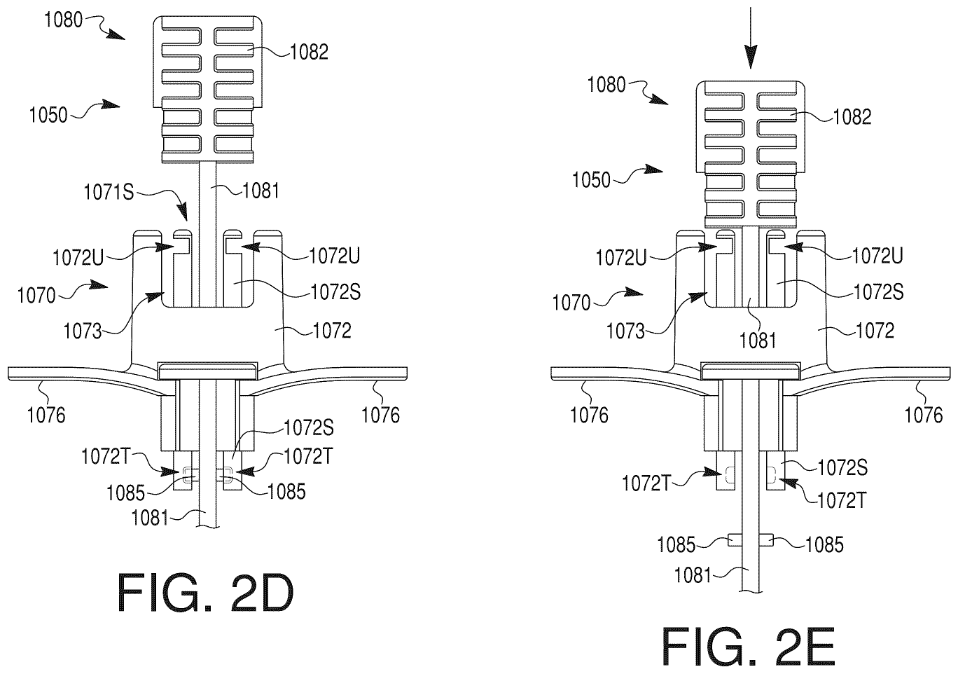

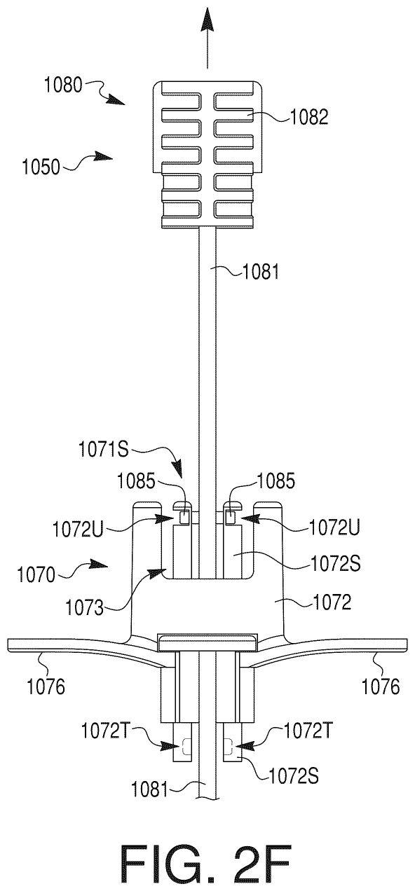

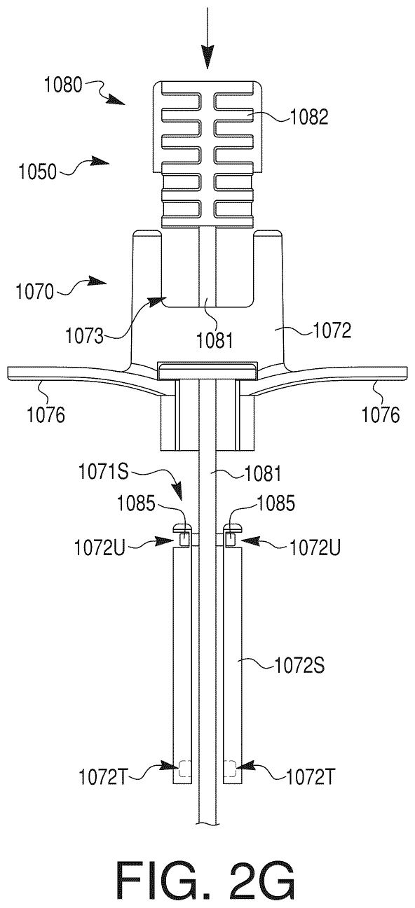

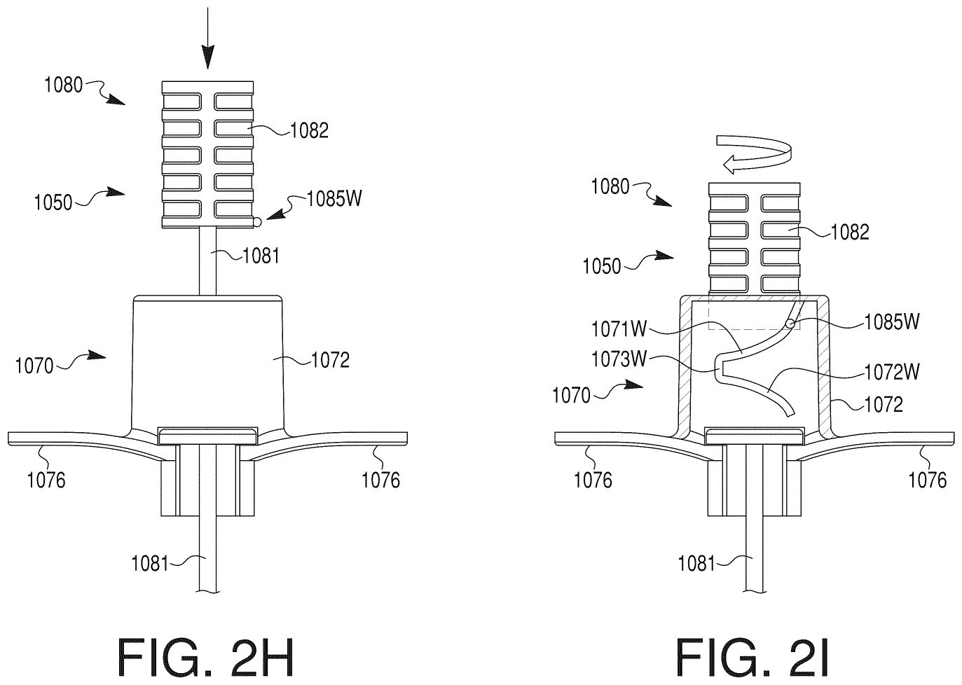

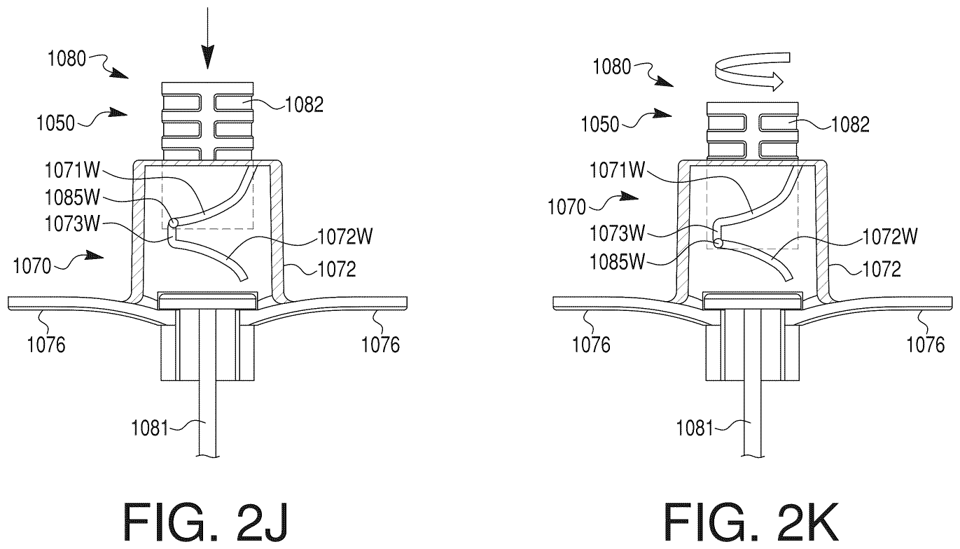

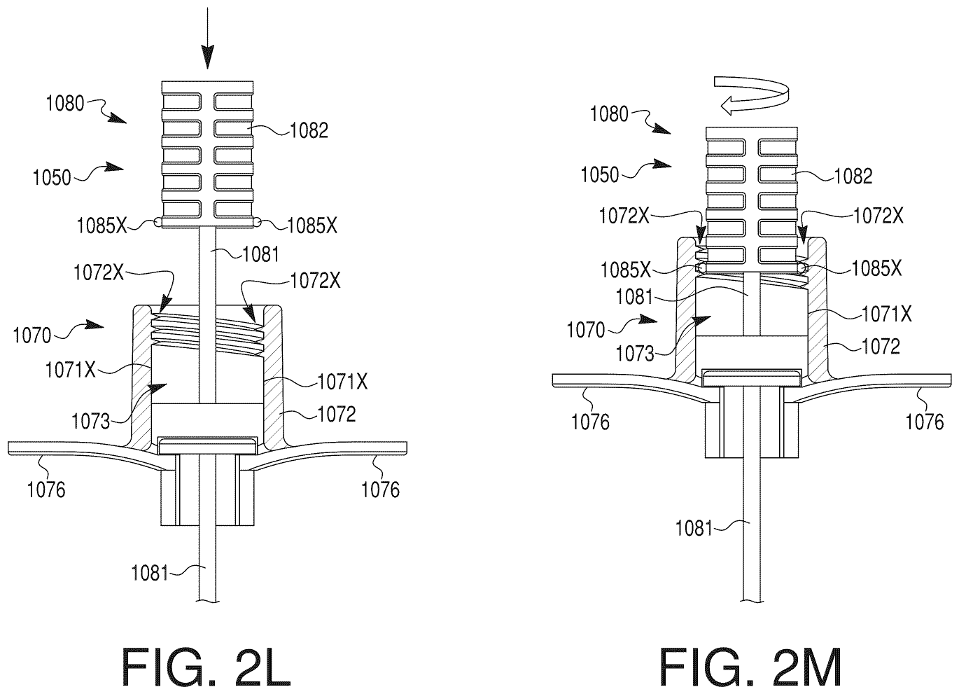

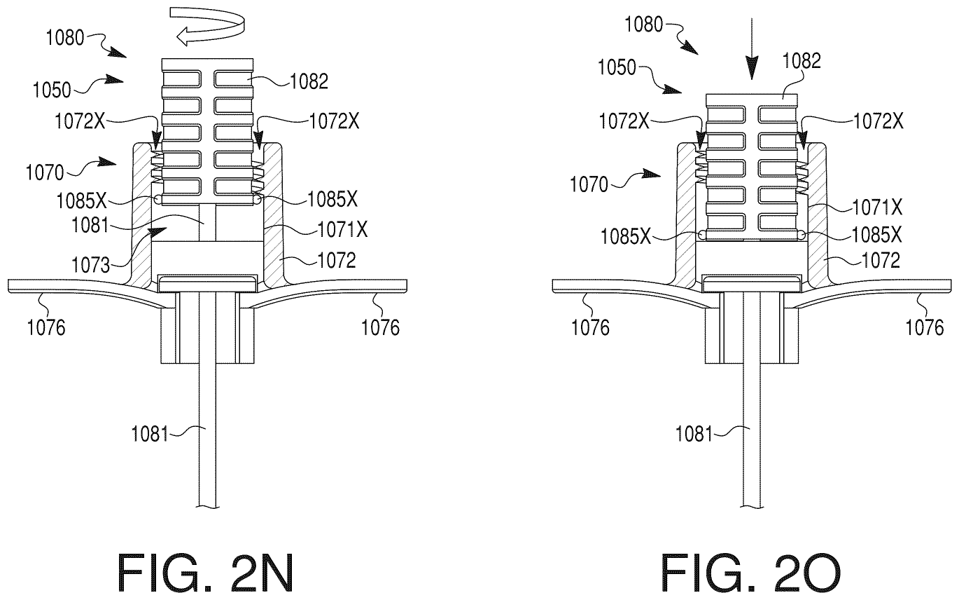

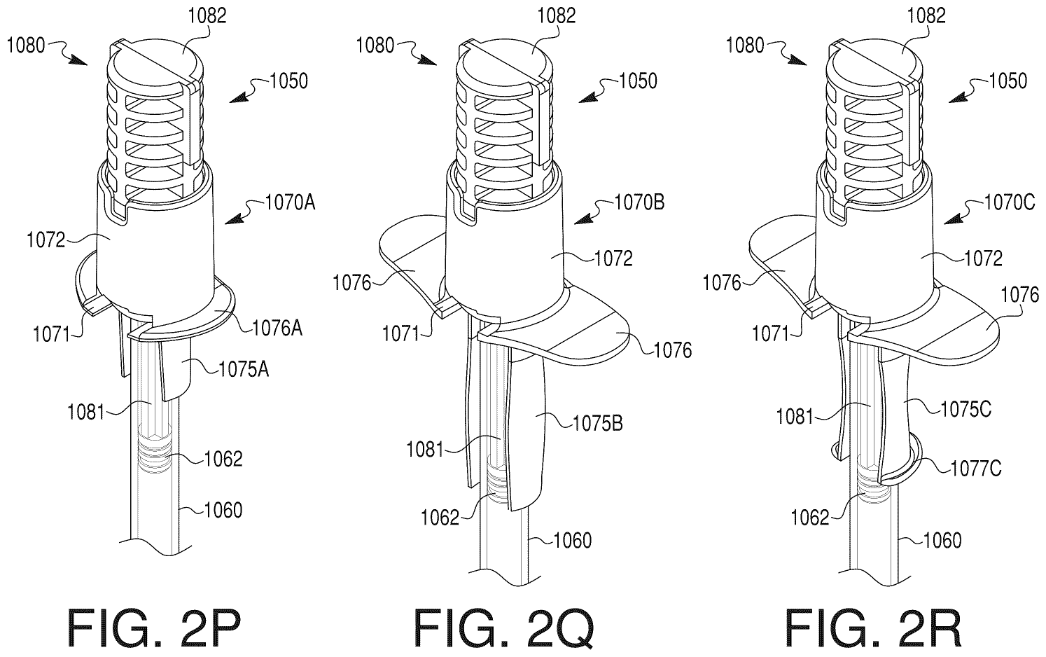

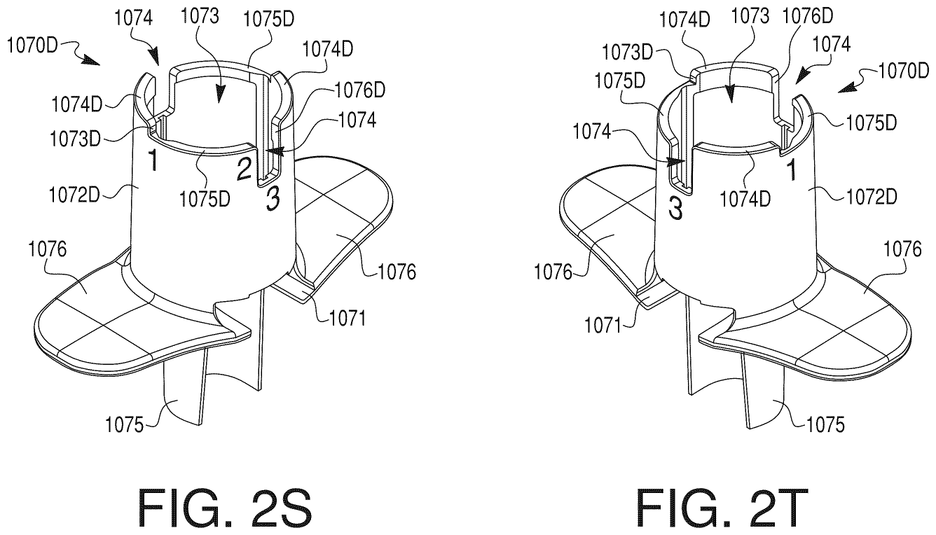

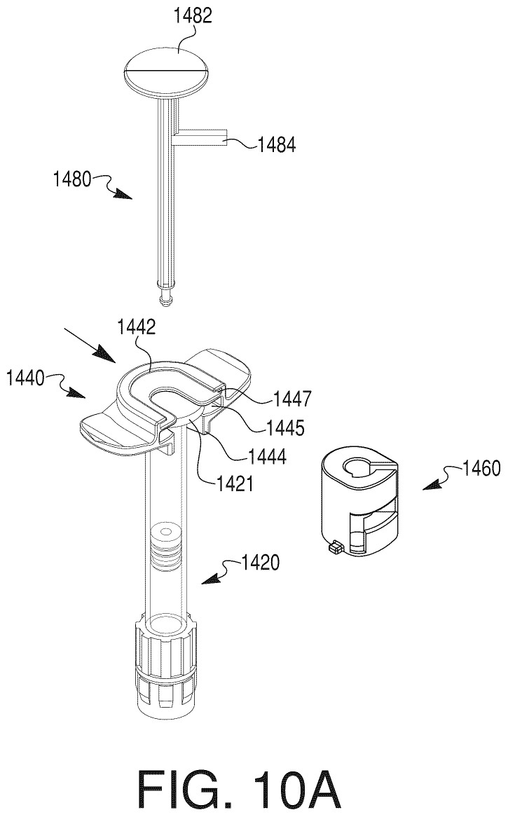

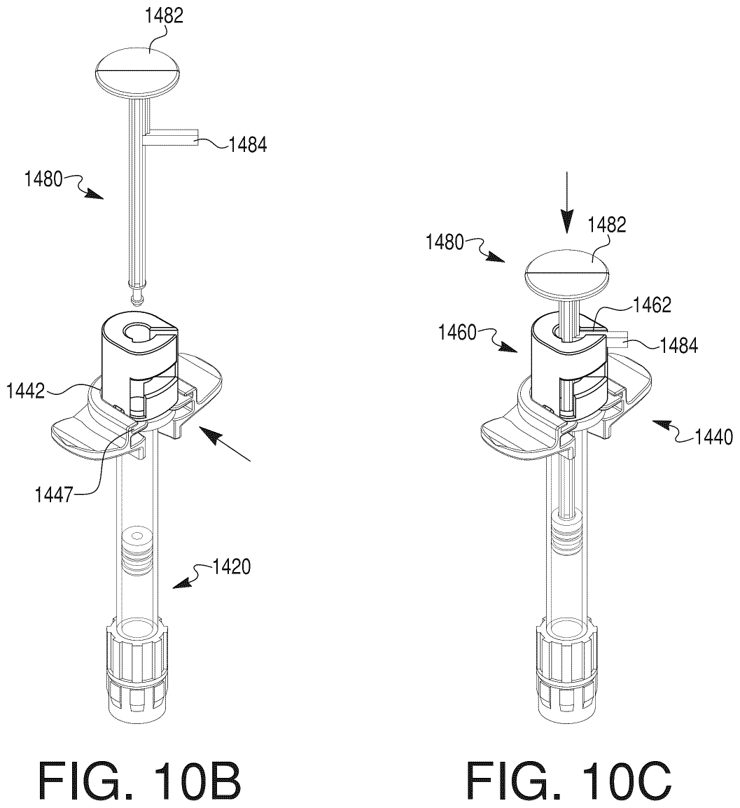

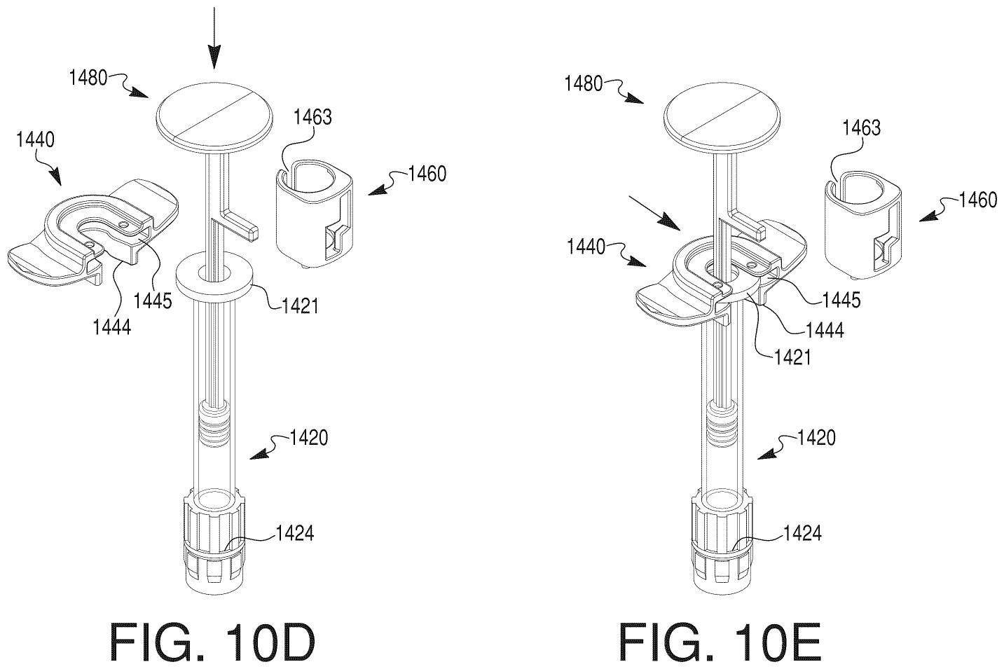

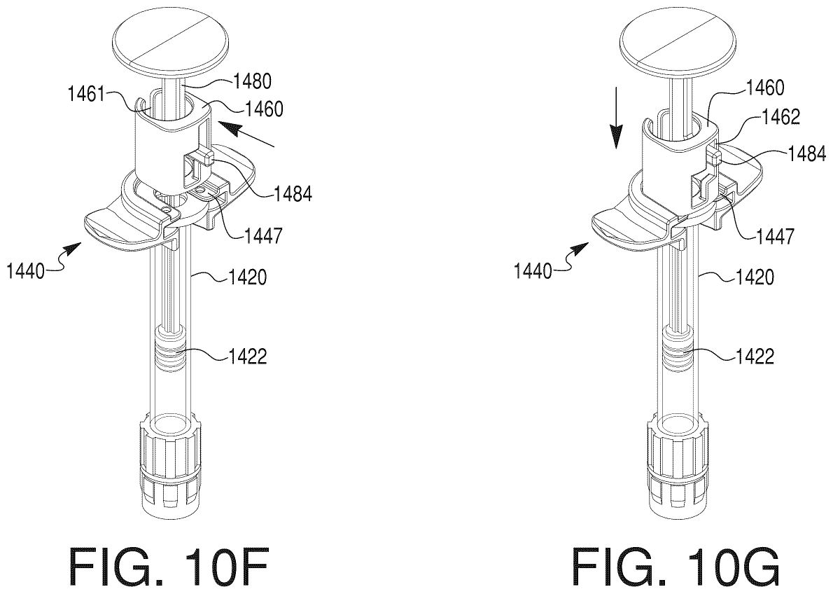

In other embodiments, as shown in FIGS. 2A-2C, flange piece 1070 may include a fixed sleeve 1072P extending proximally from collar 1072. Fixed sleeve 1072P may have a circular cross-section defining an inner channel with an opening at each terminal end of the fixed sleeve 1072P. The inner channel of fixed sleeve 1072P may extend through a longitudinal length of fixed sleeve 1072P and may be longitudinally aligned with opening 1073 (FIG. 10) such that a respective longitudinal axis of the inner channel and opening 1073 are coaxial with one another. Fixed sleeve 1072P may be sized, shaped, and configured to receive stem 1081. In some embodiments, fixed sleeve 1072P may be integral with collar 1072, while in other embodiments fixed sleeve 1072P may be a separate component assembled onto flange piece 1070.

Fixed sleeve 1072P may include a plurality of openings that are sized and shaped to receive protrusion 1085. For example, fixed sleeve 1072P may include a pair of proximal openings 1072Q and a pair of distal openings 1072R longitudinally spaced apart from one another by an offset distance. Further, the pair of proximal openings 1072Q are located at the same longitudinal position as one another, and the pair of distal openings 1072R are located at the same longitudinal position as one another. As described in further detail below, the longitudinal offset between proximal openings 1072Q and distal openings 1072R may define a dosage delivery distance for moving plunger rod 1080 to dispense a controlled volume of substance from device 1050. Alternatively, the longitudinal offset between openings 1072Q, 1072R may define a priming distance of device 1050 such that protrusion 1085 may be initially received within proximal openings 1072Q during an assembly of device 1050 to inhibit proximal retraction of plunger rod 1080. In this instance, a dosage delivery distance may correspond to a longitudinal offset between a distal end of actuation portion 1082 and a bottom surface of collar 1072 when protrusion 1085 is received within distal opening 1072R. Although not shown, it should be appreciated that an additional set of openings may be included on fixed sleeve 1072P (e.g., proximal of proximal openings 1072Q, distal of proximal openings 1072Q, and/or distal of distal openings 1072R) to further define a priming distance and/or dosage delivery distance.

A proximal end of fixed sleeve 1072P may include an angled interface 1071P defining a proximal opening of fixed sleeve 1072P. Angled interface 1071P may be tapered radially-inward toward the inner channel of fixed sleeve 1072P and configured to guide stem 1081 and protrusion 1085 into the inner channel. In the present example, protrusion 1085 may extend radially outward from stem 1081 in opposing lateral directions and may be compressible and/or formed of a flexible/deformable material, such that protrusion 1085 is configured to retract or deform radially inward into and/or toward stem 1081 in response to a force being applied thereto. In other embodiments, protrusion 1085 may be configured to at least partially deform fixed sleeve 1072P to facilitate movement of protrusion 1085 toward and/or between openings 1072Q, 1072R. In this instance, fixed sleeve 1072P may be formed of a flexible material operable to flex radially-outward when applying a distally-directed force onto stem 1081, thereby causing protrusion 1085 to apply a radial force onto fixed sleeve 1072P.

Still referring to FIG. 2A, fixed sleeve 1072P may be configured to receive plunger rod 1080 through the inner channel and allow stem 1081 to pass through collar 1072 to prime device 1050. Protrusion 1085 may be received within fixed sleeve 1072P in response to encountering angled interface 1071P and compressing radially inward relative to stem 1081 until plunger rod 1080 is moved distally enough so that protrusion 1085 is received by proximal openings 1072Q. As shown in FIG. 2B, protrusion 1085 may be configured to expand radially outward (decompress) when longitudinally aligned with proximal openings 1072Q to lock stem 1081 relative to flange piece 1070. In this instance, device 1050 may be in a primed position such that further translation of stem 1081 distally relative to fixed sleeve 1072P and flange piece 1070 may deliver a dose from device 1050. Alternatively, device 1050 may be preassembled with protrusion 1085 received in proximal openings 1072Q such that translation of stem 1081 distally relative to fixed sleeve 1072P may prime device 1050 until protrusion 1085 is received within distal openings 1072R.

As seen in FIG. 2C, while protrusion 1085 is positioned within proximal openings 1072Q applying a distally-directed force onto stem 1081 may cause fixed sleeve 1072P to compress (or deform) protrusion 1085 radially inward, thereby allowing stem 1081 to translate distally relative to fixed sleeve 1072P. Alternatively, protrusion 1085 may be manually compressed (or deformed) by applying a radially inward-directed force through proximal openings 1072Q. Protrusion 1085 may move distally through an inner channel of fixed sleeve 1072P and may be received by distal openings 1072R. As stem 1081 translates distally relative to collar 1072, device 1050 may transition from the primed position to a dose completion position when protrusions 1085 are received within distal openings 1072R, thus delivering the dose.

It should be appreciated that a volume of the dose delivered by device 1050 may be controlled based on the longitudinal offset distance between proximal openings 1072Q and distal openings 1072R. In some embodiments, fixed sleeve 1072P may include additional openings for receiving protrusion 1085 after priming and delivering a dose to inhibit proximal retraction of stem 1081 (e.g., pull back of plunger rod 1080) relative to flange piece 1070. For example, protrusion 1085 may be received within proximal openings 1072Q during an assembly of device 1050 at a manufacturing stage such that distal openings 1072R may define a priming position and a third set of openings (not shown) distal to distal opening 1072R may define a dosage delivery position. Alternatively, a bottom, interior surface of flange piece 1070 distal to distal opening 1072R may define the dosage delivery position of plunger rod 1080.

In some embodiments, as seen in FIGS. 2D-2G, flange piece 1070 may include a movable sleeve 1072S extending distally and proximally from collar 1072. Movable sleeve 1072S may have a circular cross-section defining an inner channel with an opening at each terminal end of movable sleeve 1072S. The inner channel of movable sleeve 1072S may extend through a longitudinal length of movable sleeve 1072S. Movable sleeve 1072S may be sized, shaped, and configured to be received through opening 1073, and the inner channel of movable sleeve 1072S may be sized to receive stem 1081. Movable sleeve 1072S may be fixed relative to collar 1072 when in an preassembled configuration and may be movable relative to collar 1072 upon engagement with plunger rod 1080.

Movable sleeve 1072S may include a plurality of openings that are sized and shaped to receive protrusion 1085. For example, movable sleeve 1072S may include a proximal opening 1072U at a proximal end of movable sleeve 1072S and a distal opening 1072T at a distal end of movable sleeve 1072S. A proximal end of movable sleeve 1072S may further include an angled interface 1071S defining a proximal opening of movable sleeve 1072S. Angled interface 1071S may be tapered radially-inward toward the inner channel of movable sleeve 1072S and configured to guide stem 1081 and protrusion 1085 into the inner channel of movable sleeve 1072S. In some embodiments, protrusion 1085 may extend radially outward from stem 1081 in opposite directions and may be compressible such that protrusion 1085 is configured to compress into and/or toward stem 1081 in response to a force being applied thereto.

Still referring to FIG. 2D, the proximal end of movable sleeve 1072S may be positioned adjacent to a proximal end of collar 1072 and a distal end of movable sleeve 1072S may be positioned adjacent to a distal end of collar 1072 when in the preassembled position. Plunger rod 1080 may be received through the inner channel of movable sleeve 1072S with stem 1081 extending through collar 1072. Protrusion 1085 may be received within distal opening 1072T such that plunger rod 1080 may be fixed to movable sleeve 1072S. Protrusion 1085 may be configured to exit distal opening 1072T and expand laterally outward in response to plunger rod 1080 translating relative to movable sleeve 1072S.

For example, as shown in FIG. 2E, applying a distally-directed force onto actuation portion 1082 may cause protrusion 1085 to compress radially inward, thereby allowing stem 1081 to translate distally relative to movable sleeve 1072S. In this instance, protrusion 1085 may exit distal opening 1072T and expand upon translating distally from a distal end of movable sleeve 1072S. Device 1050 may transition from a preassembled state to a primed state, in response to stem 1081 translating distally relative to collar 1072, until actuation portion 1082 abuts against a proximal end of movable sleeve 1072S. In this instance, device 1050 may be in a primed state and further translation of stem 1081 relative to collar 1072 may be inhibited by the presence of movable sleeve 1072S.

Referring now to FIG. 2F, plunger rod 1080 may couple to movable sleeve 1072S in response to proximal translation of stem 1081 relative to collar 1072 until protrusion 1085 engages proximal opening 1072U. It should be understood that protrusion 1085 may be in a compressed state when translating through an inner channel of movable sleeve 1072S and may expand into proximal opening 1072U upon longitudinal alignment therewith. With protrusion 1085 engaged to proximal opening 1072U, a distal translation of plunger rod 1080 relative to flange piece 1070 may provide a simultaneous movement of movable sleeve 1072S relative to collar 1072. It should be appreciated that a collective length of movable sleeve 1072S and plunger rod 1080 may be greater than a longitudinal length of plunger rod 1080 alone.

As seen in FIG. 2G, plunger rod 1080 may be configured to move movable sleeve 1072S through a channel of flange piece 1070 by a predetermined distance until actuation portion 1082 encounters a proximal end of collar 1072. Plunger rod 1080 may be configured to deliver a dose from device 1050 in response to translating movable sleeve 1072S distally relative to collar 1072. It should be appreciated that the dosage delivered by device 1050 may be controlled based on the predetermined distance between actuation portion 1082 and collar 1072 when protrusion 1085 is received within proximal opening 1072U. In some embodiments, flange piece 1070 may be configured to inhibit proximal movement of movable sleeve 1072S relative to collar 1072 when protrusion 1085 is received within proximal opening 1072U. Although not shown, flange piece 1070 may include one or more blocking components operable to restrict proximal retraction of movable sleeve 1072S from opening 1073.

In other embodiments, as seen in FIGS. 2H-2M, plunger rod 1080 may include at least one protrusion 1085W positioned on actuation portion 1082. In the example, protrusion 1085W may be positioned at or adjacent a distal end of actuation portion 1082 such that protrusion 1085W may be received within flange piece 1070 in response to translation of plunger rod 1080 into collar 1072.

As seen in FIG. 2K, flange piece 1070 may include one or more channels formed along an inner surface of collar 1072. In particular, collar 1072 may include a first (proximal) helical channel 1071W formed along an interior of collar 1072 and having a first curvature, and a second (distal) helical channel 1072W formed along the interior of collar 1072 and having a different and/or opposite curvature than the first helical channel 1071W. For example, when viewed from the proximal end of actuation portion 1082, first helical channel 1071W may be concave, while second helical channel 1072W may be convex when viewed from the same vantage point. Or, first helical channel 1071W may be convex when viewed from the proximal end of actuation portion 1082, while second helical channel 1072W is concave from the same vantage point. Further, second helical channel 1072W may be longitudinally spaced apart from first helical channel 1071W. First helical channel 1071W may be connected with second helical channel 1072W by an intermediate, third channel 1073W extending therebetween.

Third channel 1073W may extend along or substantially parallel to a longitudinal axis of collar 1072. It should be understood that a size, shape, and/or orientations of the one or more channels on collar 1072 are merely exemplary such that other suitable configurations may be included without departing from a scope of this disclosure. As described in detail below, the plurality of channels on collar 1072 are configured to receive protrusion 1085W. In some embodiments, first helical channel 1071W and second helical channel 1072W may be threaded and configured to mesh with a corresponding component of plunger rod 1080 (e.g., protrusion 1085W). Opposite rotational movement may be required for protrusion 1085W to traverse through first helical channel 1071W and second helical channel 1072W. For example, a first rotational movement of actuation portion (e.g., clockwise) may cause protrusion 1085W to traverse first helical channel 1071W, while an opposing rotational movement (e.g., counterclockwise) may cause protrusion 1085W to traverse through second helical channel 1072W.

Referring to FIG. 2H, with plunger rod 1080 in a ready position, protrusion 1085W may be received within collar 1072 in response to a distal translation of actuation portion 1082 toward flange piece 1070. As seen in FIG. 2I, protrusion 1085W may be received within first helical channel 1071W and moved therethrough in response to a rotation of plunger rod 1080 (e.g., in a first direction) relative to flange piece 1070. It should be appreciated that plunger rod 1080 may be configured to translate axially in a distal direction relative to flange piece 1070 as plunger rod 1080 rotates within collar 1072, due to the curvature of first helical channel 1071W. For example, plunger rod 1080 may translate a first distance defined by a configuration of first helical channel 1071W until reaching a terminal end of first helical channel 1071W. The first distance may correspond to a priming step of device 1050 such that device 1050 may be at least partially primed upon protrusion 1085W moving through first helical channel 1071W.

Referring now to FIG. 2J, protrusion 1085W may be positioned at a terminal end of first helical channel 1071W and a proximal (e.g., top) end of third channel 1073W. In some embodiments, plunger rod 1080 may experience a tactile feedback formed by the terminal end of first helical channel 1071W. Plunger rod 1080 may be translated distally through third channel 1073W to complete a priming step of device 1050, as shown in FIG. 2K. It should be understood that first helical channel 1071W and third channel 1073W may collectively define a priming distance of device 1050 such that plunger rod 1080 is in a primed position when protrusion 1085W translates through third channel 1073W.

With protrusion 1085W received within second helical channel 1072W, plunger rod 1080 may be rotated in the second direction (opposite of the first direction) to translate plunger rod 1080 distally by a second distance that is defined by a configuration of second helical channel 1072W. The second distance may be less than, greater than, and/or substantially equal to the longitudinal dimension of second helical channel 1072W, depending on the particular application and need. Plunger rod 1080 may be rotated in the second direction and translated by the second distance until reaching a terminal end of second helical channel 1072W to deliver a dose from device 1050. It should be understood that the second distance may correspond to a dosage delivery step of device 1050 such that device 1050 may deliver the dose upon protrusion 1085W moving through second helical channel 1072W and arriving at a dose completion position.

In other embodiments, as seen in FIGS. 2L-20, plunger rod 1080 may include a protrusion, a knob, and/or a thread 1085X positioned on actuation portion 1082. In the example, thread 1085X may be positioned about a circumference of actuation portion 1082 and along a distal end such that thread 1085X may be received within flange piece 1070 in response to translation of plunger rod 1080 into collar 1072.

Flange piece 1070 may further include a threaded portion 1072X disposed within opening 1073 and forming a helical path that is configured to receive thread 1085X. In the example, threaded portion 1072X may be positioned along a proximal portion of opening 1073 such that a distal portion of opening 1073 may include a non-threaded portion 1071X. As described in further detail herein, threaded portion 1072X may define a longitudinal distance corresponding to a priming step of device 1050 and non-threaded portion 1071X may define a distance corresponding to a dosage delivery step of device 1050.

For example, as seen in FIG. 2L, actuation portion 1082 may be translated distally toward flange piece 1070 until thread 1085X encounters a distal end of collar 1072. Rotation of plunger rod 1080 in a first direction (e.g., clockwise or counter clockwise) may cause thread 1085X to engage threaded portion 1072X. As shown in FIG. 2M, rotation of plunger rod 1080 may provide axial/longitudinal translation of actuation portion 1082 into collar 1072 as thread 1085X moves through the helical path of threaded portion 1072X. It should be appreciated that rotation and translation of thread 1085X through threaded portion 1072X may transition device 1050 from a ready position (FIG. 2L) to a primed position (FIG. 2N). With thread 1085X disengaged from threaded portion 1072X and positioned along non-threaded portion 1071X, device 1050 may be in the primed position. In some instances, a feedback (e.g., tactile, auditory, etc.) may be generated in response to thread 1085X exiting threaded portion 1072X and/or entering non-threaded portion 1071X.

In this instance, as shown in FIG. 2O, actuation portion 1082 may be translated distally relative to flange piece 1070 to deliver a dose from device 1050 by application of a distally-directed force against actuation portion 1082. Thread 1085X may move through the distal portion of opening 1073 when thread 1085X is positioned within non-threaded portion 1071X. A longitudinal length of non-threaded portion 1071X defined between a distal end of threaded portion 1072X and a distal end of opening 1073 may control a dosage delivery of device 1050. Device 1050 may complete delivery of a dose when actuation portion 1082 engages a proximally-facing and distal surface of collar 1072 and plunger rod 1080 arrives at the dose completion position.

FIGS. 2P-2T illustrate further embodiments of a flange piece that may be configured and operable similar to flange piece 1070 shown and described above except for the differences explicitly noted herein. It should be understood that like reference numerals are used to identify like components and the flange pieces described below may be readily incorporated with one or more components of device 1050 shown and described above.

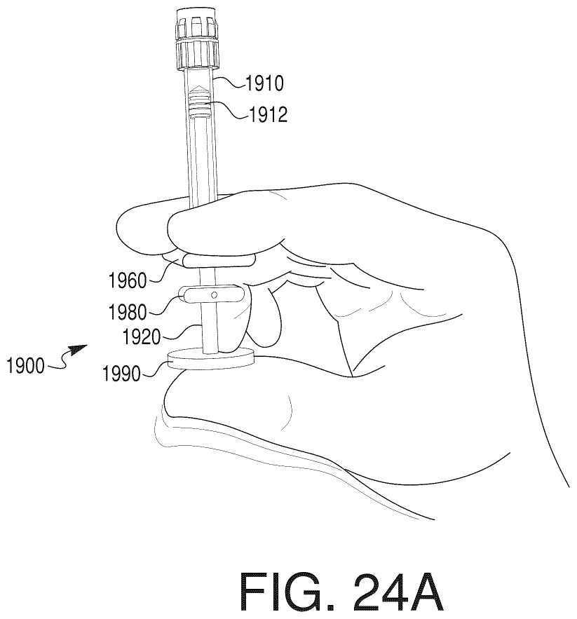

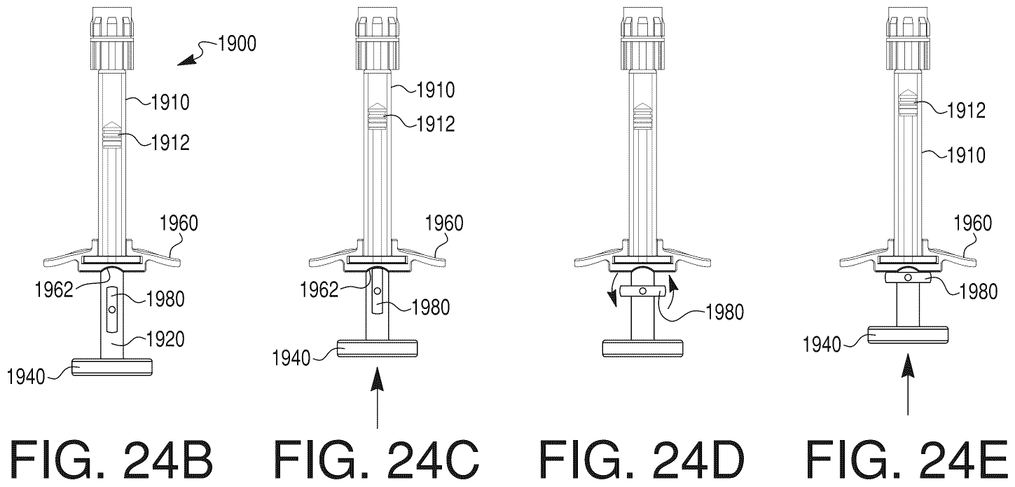

For example, referring initially to FIG. 2P, a flange piece 1070A may include one or more flanges 1076A that may be sized and configured to aid a user in holding device 1050 and/or expelling a formulated drug substance from device 1050. Flanges 1076A may be further sized and/or shaped to allow a user to hold device 1050 with a plurality of hand/grip positions, arrangements, and/or orientations. By way of illustrative example, flanges 1076A may be sized and/or shaped such that flange piece 1070A may be held similar to a writing instrument (e.g., pencil, pen, etc.) without requiring use of flanges 1076A, or sized in accordance with the example shown in FIG. 24A such that flanges 1076A may abut against one or more fingers of a user. Flange piece 1070A may include a pair of flanges 1076A extending radially outwardly from collar 1072 in opposite radial directions relative to one another. Flanges 1076A may extend transversely from collar 1072 (e.g., flanges 1076A may include an angled surface that is sloped radially-inward in a distal direction) and configured to inhibit a user's fingers from slipping off of flange piece 1070A during use of device 1050.

Flanges 1076A may be coupled to one another to form a semi-circular profile with a minimal radius relative to collar 1072. Accordingly, flanges 1076A may form a slim profile to facilitate visualization of a target treatment site at a distal end of device 1050 (not shown) when using device 1050 from a perspective proximal of flange piece 1070A. It should be understood that flange piece 1070A may include various other quantities and/or arrangements of flange pieces 1070A than those shown and described herein without departing from a scope of this disclosure. In other embodiments, flanges 1076 may include various other suitable sizes and/or shapes.

Flange piece 1070A may further include a distal collar 1075A extending distally from collar 1072 and configured to engage body 1060 to hold flange piece 1070A in a fixed position relative to body 1060. Distal collar 1075A may be adhered to, molded, or otherwise affixed to body 1060, or may engage body 1060 via a friction fit. In the example, distal collar 1075A includes a longitudinal length that is generally less than a longitudinal length of collar 1072. In some embodiments, distal collar 1075A may be sized sufficiently small enough to facilitate adequate exposure of body 1060 for user grasp and/or manipulation during use of device 1050. Additionally, distal collar 1075A may include a material composition that is similar to and/or different from collar 1072. For example, distal collar 1075A may be formed of a flexible material such that distal collar 1075A may be configured to flex radially-outward when receiving body 1060 into flange piece 1070A and flex radially-inward once body 1060 is fully received to facilitate a snap-fit connection (without breaking distal collar 1075A). It should be appreciated that, in other embodiments, flange piece 1070A may omit distal collar 1075A entirely.

In other embodiments, as seen in FIG. 2Q, a flange piece 1070B may include a distal collar 1075B that is substantially longer than distal collars 1075, 1075A shown and described above. For example, distal collar 1075B may be enlarged with a longitudinal length that is greater than a longitudinal length of collar 1072. In the example, distal collar 1075A may be sized sufficiently large enough to encompass a substantial length of body 1060. In this instance, an exterior surface of distal collar 1075B may provide an interface for a user to grasp and/or manipulate during use of device 1050. Additionally, distal collar 1075B may include an expanded diameter that exceeds a diameter of body 1060 to provide an enhanced surface area for grasping flange piece 1070B. Stated differently, distal collar 1075B may have a widened size and/or shape to facilitate ease in gripping and/or manipulating device 1050. In the present example, distal collar 1075B may have a barrel-shape with a convex outer surface when viewed from an exterior of device flange piece 1070B.

Alternatively, as seen in FIG. 2R, a flange piece 1070C may include a distal collar 1075C that is substantially similar to distal collar 1075B and includes a longitudinal length that is greater than a longitudinal length of collar 1072. In the example, an exterior surface of distal collar 1075C may be configured to provide an interface for a user to grasp and/or manipulate during use of device 1050. Distal collar 1075C may include a slim profile with a diameter that is greater than a diameter of body 1060 such that distal collar 1075C does not substantially increase a profile of body 1060. Stated differently, distal collar 1075C may have a narrowed size relative to distal collar 1075B. In some embodiments, distal collar 1075C may include a terminal lip 1077C that extends radially outward at a distal end. Terminal lip 1077C may be sized, shaped, and configured to enhance gripping and/or manipulation of distal collar 1075C. In the present example, distal collar 1075C may have a flared-shape with a concave outer surface when viewed from an exterior of device flange piece 1070C.

In other embodiments shown in FIGS. 2S-2T, a flange piece 1070D may include a collar 1072D having a proximal lip 1074D. Proximal lip 1074D may define an irregular surface configured to interface with plunger rod 1080 when actuation portion 1082 is received by collar 1072D. For example, proximal lip 1074D may include a pair of recessed surfaces 1075D positioned along opposing sides from one another along proximal lip 1074D. In other words, recessed surfaces 1075D may be separated from one another by surfaces and/or portions of proximal lip 1074D that are not recessed. In the example, recessed surfaces 1075D may be positioned adjacent to slots 1074 and may define a pathway for moving plunger rod 1080 relative to collar 1072D for priming and delivering a dose from device 1050. In some embodiments, recessed surfaces 1075D may include a spiral configuration (e.g., have a distally-directed slope) such that recessed surfaces 1075D may be tapered in a distal direction between a first ledge 1073D and a second ledge 1076D.

In some embodiments, flange piece 1070D may include visualization mechanisms, such as, for example, one or more labels or markings disposed on collar 1072D to provide instructions to a user of device 1050. For example, the one or more labels (e.g., numbering) may indicate directions in which to rotate or otherwise move plunger rod 1080 relative to flange piece 1070D to prime and deliver a dosage from device 1050. By way of example, the one or more labels may include markings that indicate a start position (e.g., “1”), a priming position (e.g., “2”), and a dosage delivery position (e.g., “3”) of protrusions 1086 relative to proximal lip 1074D. The one or more labels may be adhered, printed, embossed, and/or molded onto collar 1072D.

As described in greater detail herein, flange piece 1070D may be configured to allow movement of plunger rod 1080 in a single direction when priming and delivering a dosage from device 1050. In exemplary use, plunger rod 1080 (not shown) may initially be received through flange piece 1070D and actuation portion 1082 may be positioned against collar 1072D with protrusions 1086 positioned along a first end of recessed surfaces 1075D at marking “1” and opposite of slot 1074. Protrusions 1086 may only be rotated in a single direction along recessed surface 1075D, toward marking “2,” due to first ledge 1073D inhibiting protrusions 1086 from moving in an opposite direction away from marking “2”.

When protrusions 1086 are received along recessed surfaces 1075D at marking “2,” second ledge 1076D may further prevent protrusions 1086 from moving past slots 1074 and passing by marking “3”. It should be appreciated that a configuration of proximal lip 1074D is exemplary such that flange piece 1070D may include various other sizes, shapes, and/or configurations of proximal lip 1074D and/or recessed surfaces 1075D than those shown and described herein to facilitate movement of plunger rod 1080 during use of device 1050.

In other embodiments, the components of device 1050 may include one or more color indicators in lieu of and/or in addition to the markings described above to provide instructions to a user of device 1050. For example, device 1050 may include colors, symbols (e.g., arrows), and the like indicating a direction in which to rotate or otherwise move plunger rod 1080 relative to flange piece 1070D to prime and deliver a dosage. In one embodiment, an exterior surface of plunger rod 1080 may be provided with different colors along various portions of actuation portion 1082 to indicate a respective start position (e.g., green), priming position (e.g., yellow), and dosage delivery position (e.g., red) of plunger rod 1080 relative to collar 1072D. The one or more color indicators may be printed or molded onto plunger rod 1080. In other embodiments, the various portions of plunger rod 1080 may include different textures in lieu of and/or in addition to the color indicators described above to provide instructions to a user of device 1050.

Components of device 1050, and/or any other device described herein, may be made of any suitable material, and each component may be made from the same or different materials as other components. It should be appreciated that, in some embodiments, one or more components of device 1050 (e.g., flange piece 1070, proximal collar 1072, plunger rod 1080, actuation portion 1082, and more) may be formed of a flexible material having sufficient flexibility to prevent breakage during flexing. In some embodiments, the one or more components of device 1050 may be rigid and have enough strength to maintain shape and provide support. In other embodiments, one or more components of device 1050 (or at least a portion of a component) may having a varying rigidity along a longitudinal length or lateral width such that the component may have a variable flexibility. In still further embodiments, the one or more components of device 1050 may have sufficient flexibility to prevent breakage during flexing while also having sufficient rigidity and strength to maintain shape and provide support. In some embodiments, such features may further provide a user feedback (e.g., tactile, audible, visual, etc.) when flexing and/or interacting with other components of device 1050. For example, each of body 1060, flange piece 1070, and plunger rod 1080 may be made of a material including a polymer, such as a plastic. In some embodiments, one or more of body 1060, flange piece 1070, and plunger rod 1080 may include multiple different materials (e.g., glass, rubber, and/or plastic). In some embodiments, for example, the cylindrical portion of body 1060 may be made of glass, Plexiglas, or any other suitable polymer (e.g., cyclic olefin polymer or cyclic olefin copolymer) or other material, and stopper 1062 may be made of, e.g., plastic, rubber, or other polymer or copolymer. Body 1060 may be configured to include an interior volume of approximately 0.4 mL, 0.5 mL, or 0.6 mL. In some embodiments, stopper 1062 may be separated from plunger rod 1080 to ensure that stopper 1062 can be moved distally through body 1060 and/or prevent stopper 1062 from moving proximally through body 1060. It should be appreciated that stopper 1222 of delivery device 1050, 2800 may be similarly positioned within body 1220 relative to plunger rod 1080. By way of further example, flange piece 1070, 2870 may include polycarbonate/acrylonitrile butadiene styrene, polypropylene homopolymer, an ABS (Acrylonitrile, Butadiene, and Styrene) polymer, ABS polycarbonate blend, and other suitable materials. In some embodiments, plunger rod 1080 may include an ABS polycarbonate blend. In some embodiments, plunger rod 1080, 2880 may be formed of one or more materials, including polycarbonate/acrylonitrile butadiene styrene, polypropylene homopolymer, an ABS (Acrylonitrile, Butadiene, and Styrene) polymer, ABS polycarbonate blend, and other suitable materials. Body 1220 may be formed of Type 1 borosilicate glass. Stopper 1222 may be formed of bromobutyl rubber. A tip cap of the delivery device may be formed of polyisoprene. In further embodiments, one or more components of the delivery device may be evaluated for one or more of cytotoxicity, sensitization, irritation or intracutaneous reactivity, material mediated pyrogenicity, acute system toxicity, and/or hemocompatability.

Such materials may provide greater tolerances for manufacturing (e.g., injection molding) flange piece 1070 and/or plunger rod 1080, or facilitate an increased reproducibility of said components of device 1050. As described in greater detail above, in some embodiments, one or more components of device 1050 may be formed of a flexible and/or deformable material composition providing greater tolerances for flexing or deforming said components (e.g., without breaking) when priming or delivering a dose from device 1050.

In some embodiments, a portion of body 1060 configured to contain a formulated drug substance may be made of a transparent or translucent material. In some embodiments, flange piece 1070 and plunger rod 1080 may be made of the same, similar, or different materials, such as similar or different plastics (e.g., each having a similar or different hardness). In some embodiments, parts of device 1050 may include elastic materials. For example, parts of device 1050 may include rubber or plastic configured to allow a user to better grip device 1050, or to create an airtight or otherwise sealing fit between two components of device 1050 (e.g., between body 1060 and stopper 1062). In some embodiments, some or all of plunger rod 1080 (e.g., actuation portion 1082 and/or extensions 1087, or alternately the entirety of plunger rod 1080) may be made of a material having some flexibility, e.g., to allow for bending of extensions 1087. One or more of the materials listed above (e.g., plastic, rubber, polymers, or copolymers) may have such characteristics. In some embodiments, some or all of device 1050 may be suitable for sterilization, e.g., heat or chemical sterilization.

FIGS. 3A and 3B depict an exemplary method of assembling the delivery device depicted in FIGS. 1A-IE. Flange piece 1070 may be assembled to body 1060, as shown in FIG. 3A. The assembly of flange piece 1070 to body 1060 may include sliding, snapping, adhering, or otherwise affixing the two components together. As depicted in FIG. 3A, flange piece 1070 may be slid onto body 1060, e.g., such that lip 1071 of flange piece 1070 engages with body flange 1061. Plunger rod 1080 may be inserted through the assembled flange piece 1070 and body 1060, such that a distal end of plunger rod 1080 contacts stopper 1062. The assembled device 1050 may then be in a configuration suitable for packaging, sterilization, and/or use.

FIGS. 3C-3F depict an exemplary method of assembling device 1050 in which actuation portion 1082 includes extensions 1087 and collar 1072 includes side openings 1094. In such an embodiment, plunger rod 1080 may be inserted through flange piece 1070 until the hook or clip portions of extensions 1087 are received within side openings 1094, at which point the assembled device 1050 may be in a configuration suitable for packaging, sterilization, and/or use. It should be appreciated that side openings 1094 may be configured to inhibit a proximal retraction of plunger rod 1080 relative to flange piece 1070 once the hook or clip portions of extensions 1087 are received therein. Side openings 1094 may function as a first lock when device 1050 is placed into an initial assembly state to prevent disassembly of device 1050.

As described in further detail herein (see FIGS. 4G-4J), side openings 1095 may be configured to inhibit a proximal retraction of plunger rod 1080 once the hook or clip portions of extensions 1087 are received therein. Side openings 1095 may function as a second lock when device 1050 is placed in a dosage delivery state to prevent extracting patient fluid after completion of drug/medicament delivery. It should be appreciated that side openings 1094, 1095 may generate a feedback indicating a relative position of plunger rod 1080 to flange piece 1070, such as, for example, an audible feedback, a tactile feedback, and the like. In some embodiments, device 1050 may include additional and/or fewer side openings 1094, 1095 than those shown and described herein to increase and/or decrease a quantity of locks on device 1050.

In some embodiments, assembling device 1050 may include pre-filling body 1060 before combining it with flange piece 1070 and stopper 1062; for example, a predetermined amount of drug substance may be disposed in body 1060 between stopper 1062 and expulsion end 1064. In some embodiments, an alternate order of assembly of the components of device 1050 may be employed, depending on contemplated variations in the structures of components of device 1050. For example, in an embodiment (not shown) in which flange piece 1070 is configured to be assembled to body 1060 using a snap-fit interface, plunger rod 1080 may be first inserted through flange piece 1070, and the combined flange piece 1070 and plunger rod 1080 may be assembled to body 1060, e.g., such that flange piece 1070 snaps over a proximal body flange 1061 of body 1060 and plunger rod 1080 is inserted into body 1060.

FIGS. 4A-4F depict an exemplary method of using device 1050, according to aspects of the present disclosure. In a pre-use configuration depicted in FIG. 4A, device 1050 may hold a volume of a drug substance in between stopper 1062 and expulsion end 1064. A priming distance p may exist between protrusions 1086 and a proximal end of proximal collar 1072, and protrusions 1086 may be non-aligned with slots 1074. In a priming step depicted in FIG. 4B, plunger rod 1080 may be moved longitudinally further into body 1060. For example, a user may press actuation portion 1082 partially into proximal collar 1072 of flange piece 1070. In some embodiments, device 1050 may be held in an inverted position during this step, to ensure that air trapped in body 1060 may be expelled via expulsion end 1064, as stopper 1062 is pushed distally by plunger rod 1080. In the pre-use configuration of FIG. 4A and during the priming step shown in FIG. 4B, plunger rod 1080 may be prevented from rotating about the longitudinal axis of the syringe, due to the geometries of opening 1073 in flange piece 1070, and neck 1084 of plunger rod 1080 (as shown in the top cross-sectional view in FIG. 4B). As shown in FIG. 4C, the priming step may be stopped when protrusions 1086 of plunger rod 1080 abut a proximal end of proximal collar 1072. When the priming step is completed, neck 1084 of plunger rod 1080 may be positioned longitudinally with respect to opening 1073 of flange piece 1070, such that it may now be rotatable with respect to flange piece 1070. For example, when the priming step is completed, a narrower portion of neck 1084 may be disposed inside opening 1073 than when device was in a pre-use configuration.

As depicted in FIG. 4D, device 1050 may be in a primed configuration. In a dispensing preparation step depicted in FIG. 4E, plunger rod 1080 may be rotated about a longitudinal axis to align protrusions 1086 with slots 1074. To do so, a user may grasp and twist actuation portion 1082. In some embodiments, as has been described elsewhere, it may be possible to twist actuation portion 1082 in either direction to align protrusions 1086 and slots 1074. In other embodiments, actuation portion 1082 may be rotatable only in one direction. In some embodiments, once protrusions 1086 are aligned with slots 1074, further rotation of plunger rod 1080 relative to flange piece 1070 may be stopped by, e.g., contact between the geometries of neck 1084 and opening 1073. Thus, aligning protrusions 1086 and slots 1074 may lock device 1050 in a ready-to-dispense configuration. In some embodiments, rotation of actuation portion 1082 may align protrusions 1086 with slots 1074, and may allow plunger rod 1080 to remain longitudinally stationary relative to flange piece 1070 (e.g., no proximal or distal movement of plunger rod 1080 is caused by rotation of actuation portion 1082). As depicted in FIG. 4F, in a dispensing step, plunger rod 1080 may be moved longitudinally further into body 1060. For example, a user may press actuation portion 1082 distally into proximal collar 1072 of flange piece 1070, such that protrusions 1086 slide into slots 1074. Once protrusion 1086 abut distal ends of slots 1074, further distal movement of plunger rod 1080 is stopped. The dispensing step may ensure that a predetermined volume of a drug substance inside body 1060 is dispensed from device 1050. In some embodiments, when protrusions 1086 abut distal ends of slots 1074, stopper 1062 does not “bottom out” or abut an interior of expulsion end 1064 in body 1060. Advantageously, by ensuring that a predetermined volume of a drug substance inside body 1060 is dispensed from device 1050 before stopper 1062 can bottom out, any variations in the manufacture of expulsion end 1064 (e.g., altering the exact size or shape of expulsion end 1064) are less likely to affect the predetermined volume of drug substance that is delivered from device 1050. Indeed, in some embodiments, the predetermined volume of drug substance that is delivered from device 1050 may not be affected by typical variations in manufacturing of any component of device 1050, particularly in any component except for flange piece 1070. Advantageously, this may allow for the existence of different or larger tolerances in manufacturing variation in several components of device 1050 (e.g., variations in formation of a glass body 1060 or other glass components), without affecting the predetermined volume of drug substance to be delivered from device 1050.

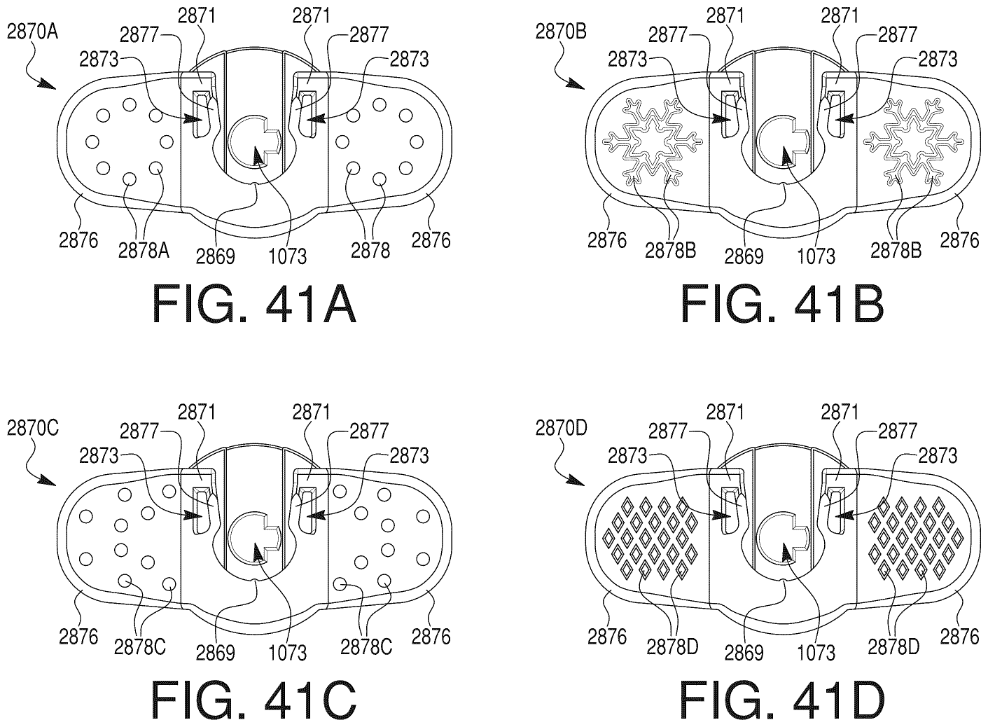

In some embodiments, after one or more steps in the use of device 1050, a user may be prevented from re-doing a step, and/or from reversing one or more steps. For example, geometries of, e.g., plunger rod neck 1084 and opening 1073 may prevent a user from pulling plunger rod 1080 proximally (e.g., out of) body 1060, from rotating plunger rod 1080 preemptively (e.g., before the priming step shown in FIG. 4C), and/or from over-rotating plunger rod 1080 during a dispensing preparation step (e.g., shown in FIG. 4E). In particular, FIGS. 4G-4J depict steps in the use of an embodiment of device 1050 having extensions 1087 on actuation portion 1082 and corresponding side openings 1094, 1095 in collar 1072 of flange piece 1070. FIGS. 4G and 4H depicts device 1050 as actuation portion 1082 is being pushed distally into collar 1072. Due to their angled distal portions, extensions 1087 are pushed inward into collar 1072. Once plunger rod 1080 has been rotated to a “delivery” position and actuation portion 1082 is further pushed distally into collar 1072 to deliver a predetermined volume of drug substance from device 1050, extensions 1087 may be received into side openings 1095 (shown in FIGS. 41 and 4J), thereafter restricting proximal movement of plunger rod 1080. Advantageously, restricting proximal movement of plunger rod 1080 may prevent inadvertent withdrawal of material into device 1050 from, e.g., a site into which a drug substance is delivered. In some embodiments, device 1050 may include either side openings 1094, or side openings 1095. In other embodiments, as shown in FIGS. 4G-4J, device 1050 may include both side openings 1094 and side openings 1095.

FIGS. 4K and 40 depicts in further detail exemplary aspects of a geometry of neck 1084, which may help to control movement of plunger rod 1080. For example, a proximal-most portion a of neck 1084 and stem 1081 (indicated by section d in FIG. 4K) may both have a first cross-sectional shape, as shown in FIG. 4L. This shape may allow for corresponding portions of plunger rod 1080 to move proximally/distally through an opening (e.g., opening 1073) of a blocking component (e.g., flange piece 1070), but may prevent rotation of plunger rod 1080 about a longitudinal axis. A narrow portion b of neck 1084 may have a smaller cross-sectional shape, as shown in FIG. 4M. This shape, when disposed in an opening (e.g., opening 1073) of a blocking component (e.g., flange piece 1070) may allow for unidirectional or bidirectional rotation of plunger rod 1080 about a longitudinal axis. It should be appreciated that the respective portion of neck 1084 allowing for transitional rotation of plunger rod 1080 (e.g., at narrow portion b) may have a geometry with the smallest cross-sectional shape to allow greater space for such movement, relative to the cross-sectional shapes of other portions of plunger rod 1080. A third portion c of neck 1084 may have a larger cross-sectional shape, as shown in FIG. 4N, which may correspond directly with the size and shape of an opening (e.g., opening 1073) of a blocking component (e.g., flange piece 1070). As such, proximal or distal movement of this portion of neck 1084 through opening 1073 may only be possible when plunger rod 1080 is in a specific rotational orientation relative to flange piece 1070. Moreover, plunger rod 1080 will not be rotatable while portion c of neck 1084 is disposed within opening 1073. This may ensure that, e.g., plunger rod 1080 is in a desirable position relative to flange piece 1070 (e.g., priming is complete and portion c is no longer disposed within opening 1073) before plunger rod 1080 may be rotated. Together, the various cross-sectional shapes of neck 1084 and the size and shape of opening 1073 may combine to create a specific sequence of movements of plunger rod 1080 needed to prime and deliver a drug substance from device 1050. In the example, a distal portion of opening 1073 may have the greatest cross-sectional profile relative to an intermediate and/or proximal portion of opening 1073 to accommodate the varying geometries of plunger rod 1080 therethrough (e.g., neck 1084, stem 1081, etc.).

In a further embodiment depicted in FIG. 4O, a proximal-most portion e of neck 1084 and a majority portion h of stem 1081 may both have a first cross-sectional shape, as shown in FIG. 4P. This shape may allow for corresponding portions of plunger rod 1080 to move proximally/distally through an opening (e.g., opening 1073) of a blocking component (e.g., flange piece 1070), but may prevent rotation of plunger rod 1080 about a longitudinal axis. A narrow portion of neck 1084 may have a smaller winged (or arrow-shaped) cross-sectional shape, as shown in FIG. 4Q (in a pre-rotation configuration relative to flange piece 1070) and FIG. 4R (in a post-rotation configuration relative to flange piece 1070). This “winged” shape, when disposed in an opening (e.g., opening 1073) of a blocking component (e.g., flange piece 1070) may allow for unidirectional or bidirectional rotation of plunger rod 1080 about a longitudinal axis, and may restrict or resist “backwards” rotation of plunger rod 1080 in the opposite direction after rotation has been completed (as described further with respect to FIGS. 4T-4X). Portions g and l of plunger rod 1080 may have a larger cross-sectional shape, as shown in FIG. 4S, which may correspond directly with the size and shape of an opening (e.g., opening 1073) of a blocking component (e.g., flange piece 1070). As such, proximal or distal movement of these portions of plunger rod 1080 through opening 1073 may only be possible when plunger rod 1080 is in a specific rotational orientation relative to flange piece 1070. Moreover, plunger rod 1080 will not be rotatable while portions g or l of plunger rod 1080 are disposed within opening 1073. This may ensure that, e.g., plunger rod 1080 is in a desirable position relative to flange piece 1070 at certain steps during assembly and use of device 1050, allowing for precise assembly and use of device 1050. Additionally, the “larger” cross sectional area of portions g and l may assist in preventing plunger rod “back-out”, as they will not be able to move proximally through opening 1073 unless in a particular rotational position relative to flange piece 1070. For example, after rotation of plunger rod 1080 from a “primed” position to a “delivery” position, portion g of plunger rod 1080 may not be able to move through opening 1073, thus preventing plunger rod “back-out” at that stage of use of device 1050. Together, the various cross-sectional shapes of plunger rod 1080 and the size and shape of opening 1073 may combine to create a specific sequence of movements of plunger rod 1080 needed to assemble, prime, and deliver a drug substance from device 1050.

FIGS. 4T-4X depict in further detail specific interactions between a wing-shaped part of neck 1084 and opening 1073 in flange piece 1070. Flange piece 1070 may include detents 1078 either adjacent to or within opening 1073, which may interface with wings 1089 on neck 1084. FIG. 4T depicts a cross-sectional view of neck 1084 inside opening 1073 in a pre-rotation configuration (e.g., after device 1050 has been primed but before plunger rod 1080 has been rotated to a “delivery” configuration relative to flange piece 1070). FIG. 4U depicts that, as plunger rod 1080 is rotated about a longitudinal axis, one of wings 1089 may contact one of detents 1078 (depending on the direction of rotation). As rotation continues, one of detents 1078 may cause one of wings 1089 to be compressed towards the remainder of neck 1084. When rotation is complete, the one of wings 1089 has passed the one of detents 1078 and has expanded. This expansion of a wing 1089 past detent 1078 may cause an auditory “click” feedback and/or a tactile feedback to indicate that rotation is complete, and may thereafter prevent “backwards” rotation of plunger rod 1080 relative to flange piece 1070. Wings 1089 and detents 1078 may be configured to interact in a similar fashion regardless of whether plunger rod 1080 is rotated in a clockwise or counterclockwise direction, thereby allowing for bidirectional rotation of plunger rod 1080 to move plunger rod 1080 from a “primed” position to a “delivery” position. As shown in further detail in FIG. 4X, each wing 1089 may have a rounded shape to allow for ease of rotation in one direction, and the expansion of a wing 1089 past a detent 1078 may place the wing 1089 in a position relative to detent 1078 that greatly resists or otherwise prohibits rotation in the opposite direction. Detent 1078 may have any suitable contour configured to assist unidirectional movement of a wing 1089 past detent 1078.

Advantageously, the various configurations of plunger rod 1080 described herein may allow for modeling, molding, and/or manufacturing one piece (e.g., plunger rod 1080) or two pieces (e.g., plunger rod 1080 and flange piece 1070) in order to achieve several goals—e.g., control desired plunger rod movement and assembly, reduce user error, prevent plunger rod back-out, and minimize a number of disparate parts needing to be manufactured and handled in order to assemble device 1050.

In some embodiments, as seen in FIGS. 4Y-4Z, device 1050 may include a pair of plunger rods in one kit, interchangeable with a single actuation portion 1082, or coupled to separate actuation portions 1082. For example, referring initially to FIG. 4Y, device 1050 may include a first plunger rod 1080A that is substantially similar to plunger rod 1080 shown and described above except for the differences explicitly noted herein. First plunger rod 1080A may include a stem 1081A having a longitudinal length A defined between a distal end of actuation portion 1082 and a tip 1083A. As described in detail below, longitudinal length A may define a priming distance for moving plunger rod 1080A relative to flange piece 1070 for priming device 1050. Tip 1083A may have a flat and/or planar interface that may be configured to inhibit engagement of stopper 1062 when first plunger rod 1080A is received within body 1060.

Referring now to FIG. 4Z, device 1050 may further include a second plunger rod 1080B that is substantially similar to plunger rod 1080. Second plunger rod 1080B may include a stem 1081B extending distally from actuation portion 1082 and having a longitudinal length B defined between a distal end of actuation portion 1082 and a tip 1083B. Tip 1083B is substantially similar to tip 1083A described above. Longitudinal length B of stem 1081B is relatively greater than longitudinal length A of stem 1081A and may define a dosage delivery distance for moving plunger rod 1080A relative to flange piece 1070 to deliver a dose from device 1050.

First plunger rod 1080A may be configured to prime device 1050 in response to translating stem 1081A through collar 1072 and into body 1060 (see FIGS. 1A-1B). In this instance, tip 1083A may contact and push stopper 1062 distally by the priming distance. It should be understood that the priming distance of device 1050 may be controlled based on a size of longitudinal length A of first plunger rod 1080A. Upon priming device 1050, first plunger rod 1080A may be removed from body 1060 and flange piece 1070 without retracting stopper 1062 due to a flattened-interface of tip 1083A. Accordingly, stopper 1062 may remain at a fixed position relative to body 1060 upon retraction of first plunger rod 1080A.

Second plunger rod 1080B may be configured to deliver a dose from device 1050 in response to translating stem 1081B through collar 1072 and into body 1060 (see FIGS. 1A-1B), after the priming step described above using stem 1081A. In this instance, tip 1083B may contact and push stopper 1062 distally by the dosage delivery distance. It should be understood that the dosage delivery distance of device 1050 may be controlled based on a size of longitudinal length B of second plunger rod 1080B. The dosage delivery distance may be substantially equal to the difference in length between stem 1081B and stem 1081A.

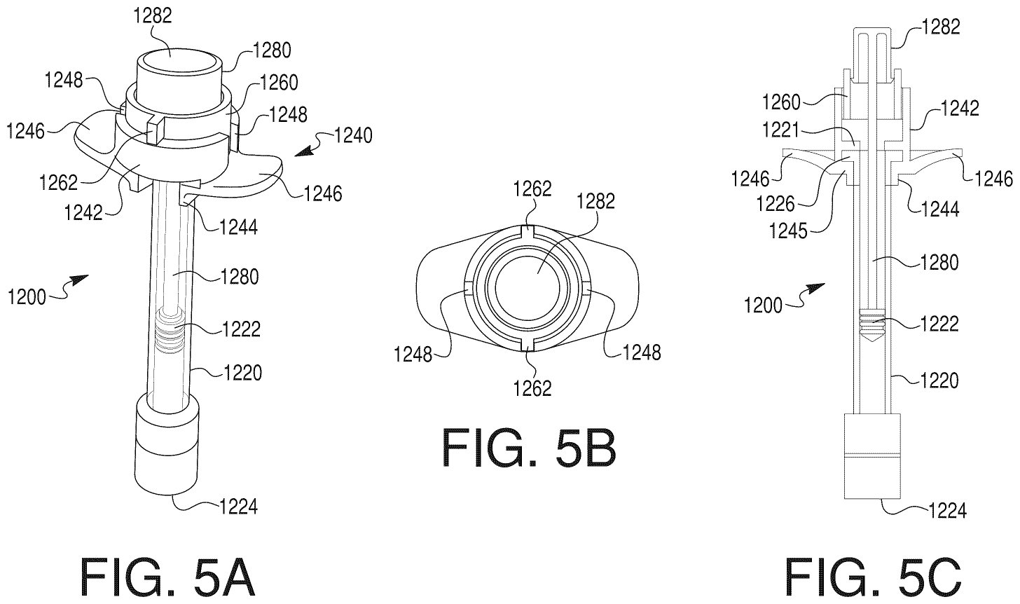

FIGS. 5A-5C depict another exemplary delivery device 1200 according to additional embodiments of the present disclosure. Device 1200 includes a body 1220, and a flange piece 1240 with a proximal collar 1242, in which an inner collar 1260 may be disposed. Together, proximal collar 1242 and inner collar 1260 may form a blocking component for device 1200. A plunger rod 1280 (also referred to herein as “stem” or “plunger rod stem”) may pass through inner collar 1260 and flange piece 1240, into body 1060. Plunger rod 1280 may share a longitudinal axis with a central axis of proximal collar 1242 and inner collar 1260, and may have an actuation portion 1282 sized and configured to fit (e.g., nest or otherwise fit) inside inner collar 1260.

Device 1200 may be, for example, an injection device, such as a syringe, for dispensing a predetermined volume of a formulated drug substance. Generally, device 1200 may share size, capacity, material, preparation, assembly, manufacturing, operation, or use characteristics with device 1050, or with other delivery devices disclosed herein. As with device 1050, device 1200 may be configured for ease of use and may include one or more features that aid a user by providing tactile, auditory, or visual feedback (e.g., using any of the features described elsewhere herein).

Body 1220 may have any or all of the same characteristics as, e.g., body 1060 of device 1050, or as any syringe body known in the art. For example, in some embodiments, body 1220 may be pre-fillable or pre-filled (e.g., fillable or filled with a drug substance prior to completed assembly, packaging, sterilization and/or shipment of device 1200 to users). In some embodiments, a stopper 1222 may be configured to be inserted into body 1220 and may be configured to hold a predetermined volume of a formulated drug substance inside body 1220, between stopper 1222 and an expulsion end 1224.

Flange piece 1240 may be of any suitable size and/or shape to close, partially close, cover, or partially cover an end of body 1220 opposite expulsion end 1224, and/or to support and hold plunger rod 1280 in place inside body 1220. In some embodiments, flange piece 1240 may share some characteristics with flange piece 1070 of device 1050. For example, flange piece 1240 may include a distal collar 1244 configured to engage with body 1220 and hold flange piece 1240 in place in relation to body 1220. For example, distal collar 1244 may include a lip 1245 that may slide over a body flange 1226, to hold flange piece 1240 in place. In alternative embodiments, lip 1245 of distal collar 1244 may be made of a flexible or semi-flexible material, so that it may snap in place over body flange 1226. In further embodiments, distal collar 1244 or another portion of flange piece 1240 may be adhered to, molded to, or otherwise affixed to, body 1220, or may engage with body 1220 via a friction fit.

In some embodiments, flange piece 1240 may include one or more flanges 1246, which may be sized and configured to aid a user in holding device 1200 and/or expelling a formulated drug substance from device 1200. In some embodiments, as depicted in FIGS. 1A-1E, flange piece 1240 may include two flanges 1246 opposite to one another and extending perpendicularly from a longitudinal dimension of device 1200. In some embodiments, flange piece 1240 may include other arrangements of a flange or flanges, such as four flanges, or one circumferential flange extending radially outward from a central longitudinal axis of device 1200. In some embodiments, flange piece 1240 may extend radially outward from a central longitudinal axis of device 1200 farther than a circumference of body 1220. In such embodiments, flange piece 1240 may support device 1200 if device 1200 is placed on a surface, may prevent device 1200 from rolling on a flat surface, and/or may allow device 1200 to be picked up more easily.

In some embodiments, flange piece 1240 and inner collar 1260 may be sized and configured to serve as a blocking component in device 1200, e.g., by limiting and/or directing rotational and longitudinal movement of plunger rod 1280. Proximal collar 1242 of flange piece 1240 may be sized and configured to accept part of inner collar 1260, while blocking protrusions 1262 from moving distally until inner collar 1260 is rotated to a particular position. In turn, inner collar 1260 may be sized and configured to receive part or all of an actuation portion 1282 of plunger rod 1280. As shown in FIGS. 5A-5C, proximal collar 1242, inner collar 1260, and actuation portion 1282 may all have generally cylindrical shapes; in alternate embodiments, each of proximal collar 1242, inner collar 1260, and actuation portion 1282 may have any suitable size or shape that allows for actuation portion 1282 to fit (e.g., nest) within inner collar 1260, and inner collar 1260 to fit within proximal collar 1242.

Plunger rod 1280 and inner collar 1260 may be in general rotatable about a shared central longitudinal axis (e.g., in one direction or in both directions). Moreover, both plunger rod 1280 and inner collar 1260 may be movable along the central longitudinal axis, e.g., in a distal direction to prime device 1200 and/or deliver a volume of drug substance from expulsion end 1224 of body 1220. Actuation portion 1282 of plunger rod 1280 may include a distal geometry which, when actuation portion 1282 is moved distally into inner collar 1260, interfaces with inner collar 1260 to prevent proximal movement (e.g., back-out) of plunger rod 1280 from inner collar 1260. For example, actuation portion 1282 may include a wedge-shaped distal portion that, when it passes a distal portion of inner collar 1260, expands distally from inner collar 1260 so that actuation portion 1282 can no longer move freely in relation to inner collar 1260.

Flange piece 1240 may include cavities, such as slots 1248, into which protrusions 1262 of inner collar 1260 may slide when inner collar 1260 is rotated to a particular position. As with slots 1074 of device 1050, slots 1248 may have a depth dimension parallel to a longitudinal axis of device 1200, and the depth of slots 1248 may correspond to a distance plunger rod 1280 must move distally in order to push stopper 1222 towards expulsion end 1224, and dispense a predetermined volume of formulated drug substance from body 1220 through expulsion end 1224.

In some embodiments, device 1200 may have additional features. For example, in some embodiments, a neck of plunger rod 1280 may have a geometry complementary to an opening of flange piece 1240 that restricts the extent and direction that plunger rod 1280 may rotate or move longitudinally, similar to neck 1084 and opening 1073 of device 1050. For example, rotation and/or longitudinal movement of plunger rod 1280 may be restricted based on priming, preparing, and/or drug delivery steps during use of device 1200. As another example, plunger rod 1280 may be prevented from being pulled or backed out of body 1220 at any point during preparation or use of device 1200.

In a contemplated method of use of device 1200, device 1200 may be filled with a predetermined volume of drug substance. The predetermined volume of drug substance may be greater than a volume of drug substance suitable for delivery to a patient. In some embodiments, device 1200 (e.g., body 1220) may contain both a predetermined volume of drug substance and an air bubble (not shown) that should be removed prior to delivery of the drug substance to a patient. In some embodiments, device 1200 may be a pre-filled syringe. In order to prime device 1200 (e.g., removing an air bubble if any and ensuring that a suitable volume of the drug substance will be delivered to a patient), a user may push actuation portion 1282 of plunger rod 1280 into inner collar 1260. A geometry of actuation portion 1282 may interact with inner collar 1260 (e.g., a distal wedge or clip of actuation portion 1282 may expand on a distal side of inner collar 1260) to secure actuation portion 1282 in and/or to inner collar 1260 and to prevent back-out of plunger rod 1280. At this point, device 1200 may be in a “primed” state. Subsequently, inner collar 1260 may be rotated about a longitudinal axis, until protrusions 1262 become longitudinally aligned with slots 1248. At this point, device 1200 may be in a “delivery” state. To deliver a predetermined volume of drug substance from device 1200, inner collar 1260, together with actuation portion 1282 and plunger rod 1280, may then be moved distally until protrusions 1262 abut a distal end of slots 1248. The distance traveled by plunger rod 1280 in this step may push stopper 1222 distally by a distance required to dispense the predetermined volume of drug substance from expulsion end 1224 of device 1200.

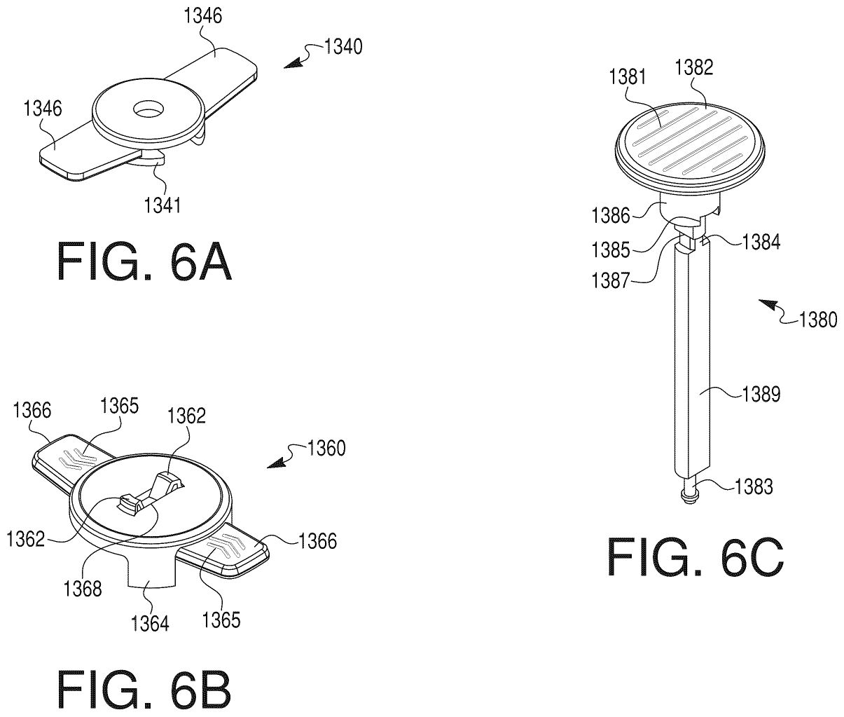

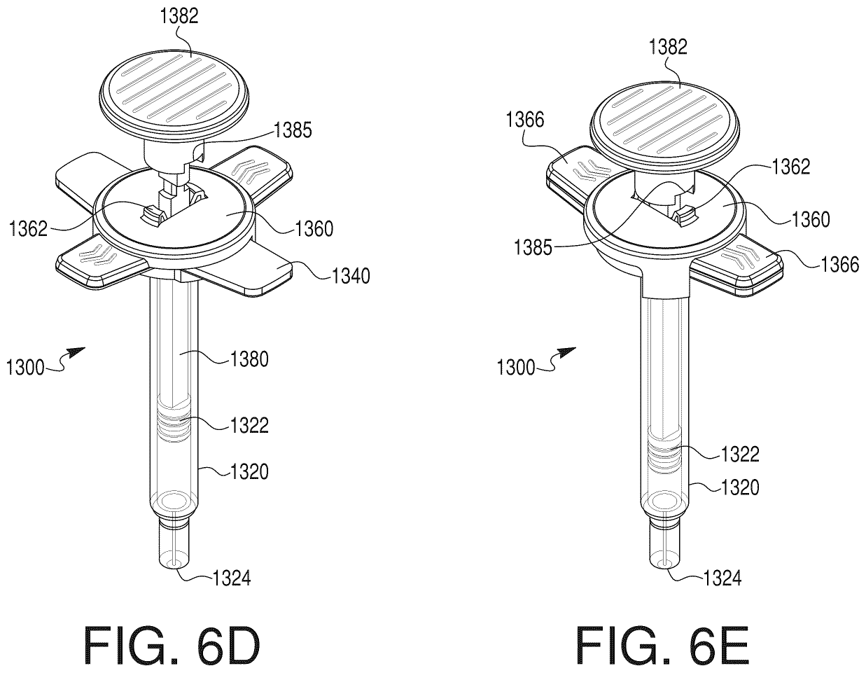

Referring now to FIGS. 6A-6E, views of a delivery device 1300 and component parts are depicted. Delivery device 1300 includes a blocking component comprising a distal flange piece 1340 and a proximal flange piece 1360, a plunger rod 1380, and a body 1320. Distal flange piece 1340 and proximal flange piece 1360 each include flanges (1346 and 1366, respectively). The flanges 1366 of proximal flange piece 1360 optionally may include a texture 1365. Distal flange piece 1340 includes a channel 1341 which may allow for distal flange piece 1340 and body 1320 to be slidably assembled. Proximal flange piece 1360 includes a clip 1364 which may allow for proximal flange piece 1360 and distal flange piece 1340 to be movably affixed to one another, such that they may still be rotatable relative to one another about a longitudinal axis of delivery device 1300 (see, e.g., FIGS. 6D and 6E). Proximal flange piece 1360 includes clips 1362 bordering a central opening 1368 through which plunger rod 1380 may pass. Plunger rod 1380 includes an actuation portion 1382, which optionally may include a texture 1381. Plunger rod 1380 further includes a distal neck portion 1384, a proximal neck portion 1387, and a proximal stop 1386 having a cavity 1385, all of which are configured to interface with distal flange piece 1340 and proximal flange piece 1360 in a plurality of configurations to allow for controlled priming and delivery of a predetermined volume of a drug substance using delivery device 1300. Plunger rod 1380 further includes a distal tip 1383 at a distal end of a stem 1389, where tip 1383 is configured to interface with stopper 1322. Tip 1383 may have any suitable size, shape, and mode of attaching to, affixing to, or pushing stopper 1322 as has been described with respect to, e.g., tip 1083 of plunger rod 1080. As with stem 1081, stem 1389 may have any size and configuration suitable to fit inside body 1320. In some embodiments, stem 1389 may be sized and configured to provide sufficient size (e.g., thickness), stability and/or rigidity to reduce a likelihood of undesirable bending, wobbling, or breaking.

Body 1320 (depicted in FIGS. 6D and 6E) may have any or all of the same characteristics as, e.g., body 1060 of device 1050, or as any syringe body known in the art. For example, in some embodiments, body 1320 may be pre-fillable or pre-filled. A stopper 1322 may be configured to be inserted into body 1320 and may be configured to hold a predetermined volume of a formulated drug substance inside body 1320, between stopper 1322 and an expulsion end 1324.

Delivery device 1300 may be, for example, an injection device, such as a syringe, for dispensing a predetermined volume of a formulated drug substance. Generally, delivery device 1300 may share size, capacity, material, preparation, assembly, or manufacturing characteristics with device 1050, device 1200, or with other delivery devices disclosed herein. As with devices 1050 and 1200, delivery device 1300 may be configured for ease of use and may include one or more features that aid a user by providing tactile, auditory, or visual feedback (e.g., textures 1365, 1381, other textures, labels, colors, or tactile or auditory feedback, or using any of the other features described elsewhere herein). As with devices 1050 and 1200, such features are optional, and one or more such features may be combined to improve ease of use.

Proximal flange piece 1360 and distal flange piece 1340 may be of any suitable size and/or shape to serve as a blocking component in delivery device 1300, to close, partially close, cover, or partially cover an end of body 1320 opposite expulsion end 1324, and/or to support and hold plunger rod 1380 in place inside body 1320. In some embodiments, proximal flange piece 1360 and distal flange piece 1340 may each include one or more flanges, which may be sized and configured to aid a user in holding device 1300 and/or expelling a formulated drug substance from expulsion end 1324. In some embodiments, as depicted in FIGS. 6A-6E, flange pieces 1360, 1340 may each include two flanges 1366, 1346 respectively, where each pair of flanges is opposite to one another and extending perpendicularly from a longitudinal dimension of device 1300. In general, other arrangements of a flange or flanges, such as one flange on each of flange pieces 1360, 1340, are possible. Each of flange pieces 1340, 1360 may extend radially outward from a central longitudinal axis of device 1300 farther than a circumference of body 1320, to, e.g., support device 1300 if device 1300 is placed on a surface, prevent device 1300 from rolling on a flat surface, and/or allow device 1300 to be picked up more easily.

Flange pieces 1360 and 1340 may, in combination, form a central opening having a changeable size and/or shape depending on a relative position of proximal flange piece 1360 and distal flange piece 1340. For example, in the configuration depicted in FIG. 6D, proximal flange piece 1360 and distal flange piece 1340 may combine to form an opening sized and configured to allow for distal passage of distal neck portion 1384 of plunger rod 1380, but to block passage of proximal neck portion 1387. In the second configuration depicted in FIG. 6E (e.g., where flanges 1346 and 1366 are in alignment), the central opening formed by flange pieces 1360 and 1340 may be sized and configured to allow for distal passage of proximal neck portion 1387. Proximal stop 1386 may be of a size and shape that is too large to pass through the central opening formed by flange pieces 1360 and 1340 in any combination. In some embodiments, distal flange piece 1340 may be assembled with body 1320 and plunger rod 1380 such that distal flange piece 1340 is not movable relative to body 1320 and not rotatable relative to plunger rod 1380. Proximal flange piece 1360 may, in contrast, be assembled to distal flange piece 1340 (and body 1320) such that it is rotatable about a longitudinal axis in relation to distal flange piece 1340, body 1320, and plunger rod 1380, which may pass through central opening 1368. Specifically, proximal flange piece 1360 may be rotatable relative to distal flange piece 1340 from a first configuration in which flanges 1346, 1366 are offset from one another (see FIG. 6D), to a second configuration in which flanges 1346, 1366 overlay one another (see FIG. 6E). One of ordinary skill in the art will understand that in alternate embodiments, distal flange piece 1340 may be rotatable in relation to other parts of device 1300, while proximal flange piece 1360 may not be rotatable. In yet further embodiments, both proximal flange piece 1360 and distal flange piece 1340 may be assembled with body 1320 and plunger rod 1380 such that they are both rotatable relative to other components of device 1300.

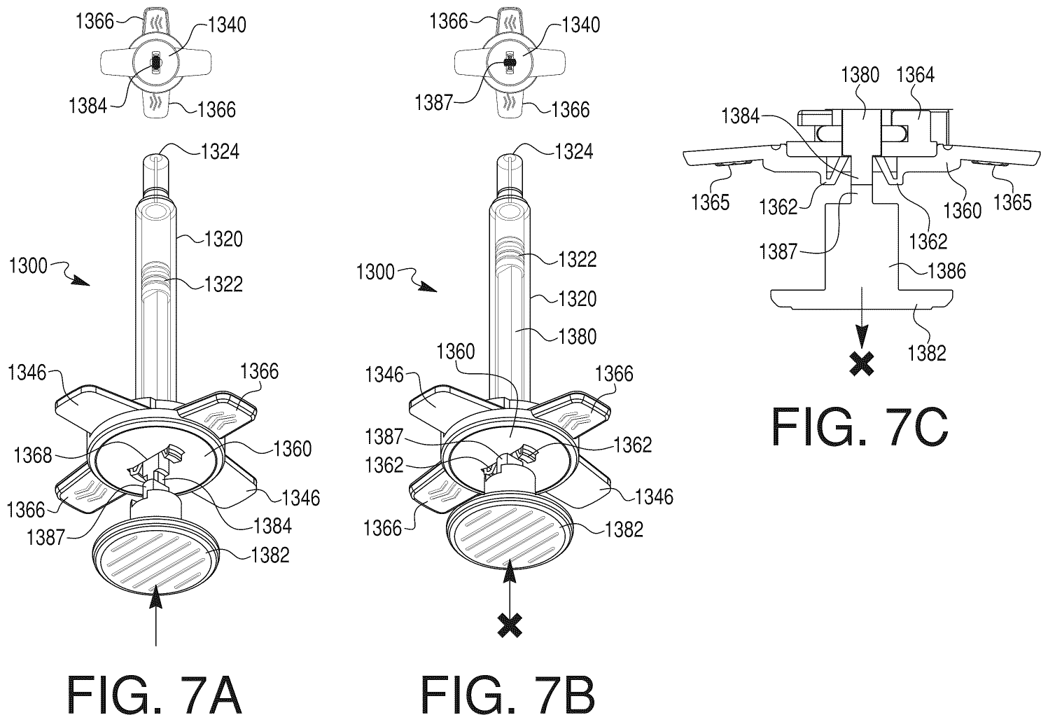

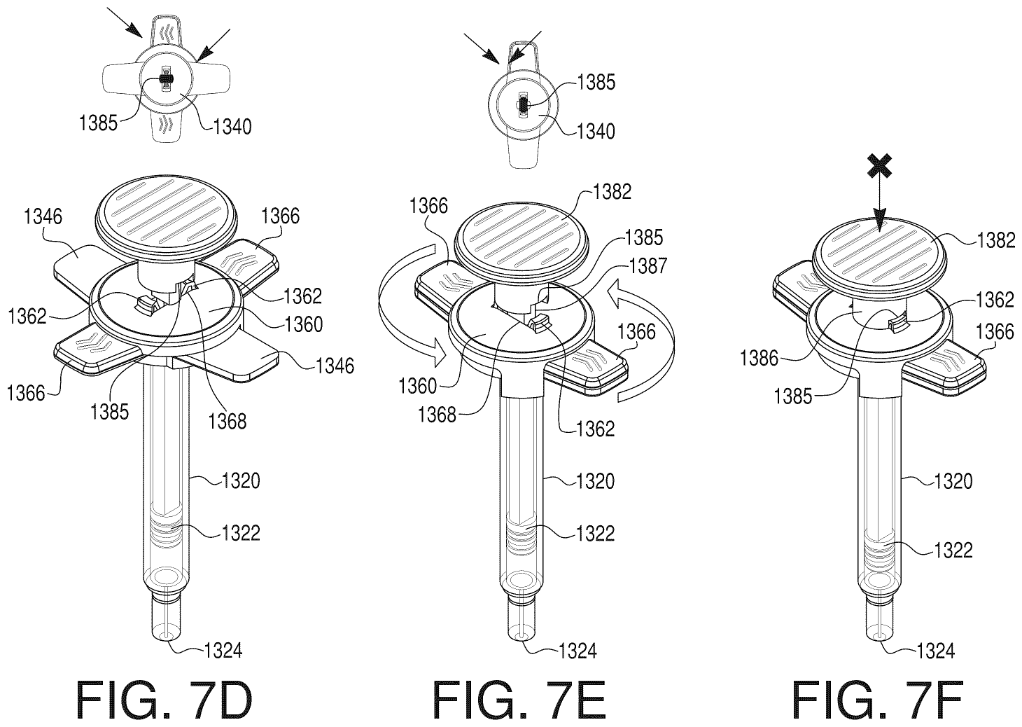

Clips 1362 of proximal flange piece 1360 may overhang and be biased towards opening 1368. In a pre-use configuration (depicted in FIG. 6D), clips 1362 may be compressed by plunger rod 1380. They may be positioned on proximal flange piece 1360 such that, upon distal movement of plunger rod 1380 such that distal neck portion 1384 passes through opening 1368, they expand inward to abut the sides of distal neck portion 1384. Once clips 1362 expand in this manner, they may block proximal movement of plunger rod 1380, e.g., to prevent plunger rod back-out (see, e.g., FIG. 7C). A cavity 1385 may be positioned on proximal stop 1386 for each clip 1362, such that when plunger rod 1380 is moved distally into body 1320 to a fullest desired extent, each clip 1362 may fit into a cavity 1385.

FIGS. 7A-7F depict an exemplary method of using device 1300, according to aspects of the present disclosure. In a pre-use configuration depicted in FIG. 7A, device 1300 may hold a volume of a drug substance in between stopper 1322 and expulsion end 1324. Flange pieces 1340 and 1360 may be in a pre-use configuration, in which flanges 1346, 1366 are offset from one another. Plunger rod 1380, which may abut or be assembled to stopper 1322, may be partially inserted into body 1320 through flange pieces 1340, 1360. Proximal flange piece 1360 may be prevented from rotating about the longitudinal axis of the syringe, due to the geometries of plunger rod 1380 and flange piece 1360. In a priming step depicted in FIG. 7B, plunger rod 1380 may be moved longitudinally further into body 1320, until distal movement is blocked by the abutment of proximal neck portion 1387 against a surface of proximal flange piece 1360. For example, a user may press actuation portion 1382 towards proximal flange piece 1360. In some embodiments, device 1300 may be held in an inverted position during this step, to ensure that air trapped in body 1320 may be expelled via expulsion end 1324, as stopper 1322 is pushed distally by plunger rod 1380. In the “primed” configuration, distal neck portion 1384 may be disposed in opening 1368 of proximal flange piece 1360. Moreover, as depicted in FIG. 7C, once the priming step is stopped, clips 1362 may be released from their compressed configuration such that they may expand inwards and abut a side of distal neck portion 1384. As distal neck portion 1384 may be comparatively narrower than the part of plunger rod 1380 previously disposed in opening 1368, the expansion of clips 1362 may prevent proximal movement (e.g., back-out) of plunger rod 1380.

As depicted in FIG. 7D, device 1300 may be in a primed configuration. In a dispensing preparation step depicted in FIG. 7E, proximal flange piece 1360 may be rotated about a longitudinal axis to align flanges 1366 and flanges 1346, and to change (e.g., enlarge) a shape of the central opening formed by the combined openings of proximal flange piece 1360 and distal flange piece 1340. To do so, a user may grasp and twist proximal flange piece 1360. In some embodiments, it may be possible to twist proximal flange piece in either direction to align flanges 1366 and flanges 1346. In other embodiments, proximal flange piece 1360 may be rotatable only in one direction. In some embodiments, once flanges 1366 and flanges 1346 are aligned (as shown in, e.g., FIG. 7E), further rotation of plunger rod 1080 relative to flange piece 1070 may be stopped by, e.g., clip 1362 abutting against flange 1346. Thus, device 1300 may be locked in a ready-to-dispense configuration. As depicted in FIG. 7F, in a dispensing step, plunger rod 1380 may be moved longitudinally further into body 1320. For example, a user may press actuation portion 1382 distally, until each of clips 1362 enter a cavity 1385 in a proximal stop 1386, and/or until proximal stop 1386 abuts a proximal surface of proximal flange piece 1360. The dispensing step may ensure that a predetermined volume of a drug substance inside body 1320 is dispensed from device 1300.

In some embodiments, after each successive step in the use of device 1300, a user may be prevented from re-doing a step, and/or from reversing one or more steps. For example, geometries of, e.g., plunger rod 1380 and the combined openings of proximal flange piece 1360 and distal flange piece 1340 may prevent a user from pulling plunger rod 1380 proximally (e.g., out of) body 1320, from rotating plunger rod 1380, from rotating proximal flange piece 1360 preemptively (e.g., before completion of the priming step shown in FIGS. 7B and 7C), and/or from over-rotating flange piece 1360 during a dispensing preparation step (e.g., shown in FIG. 7E).

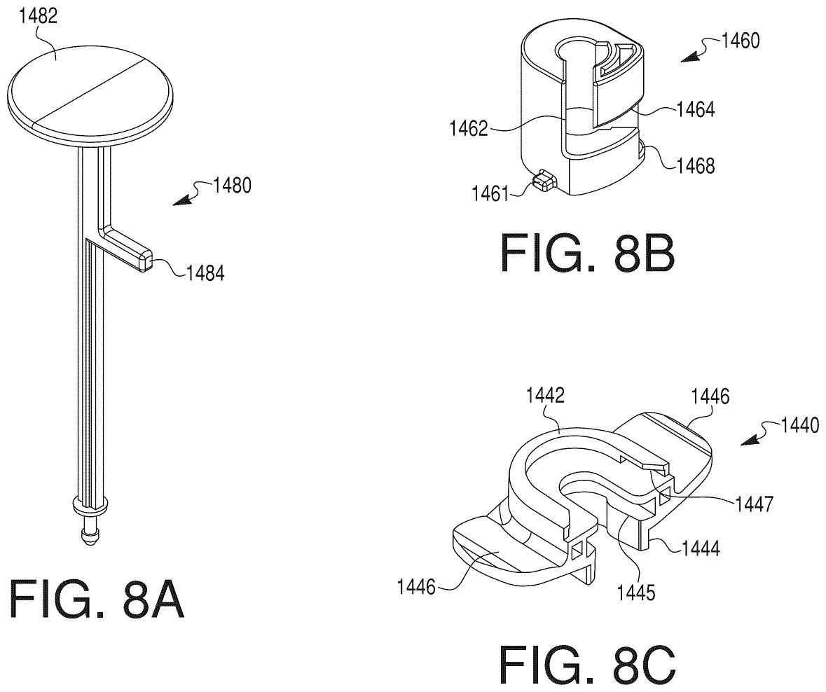

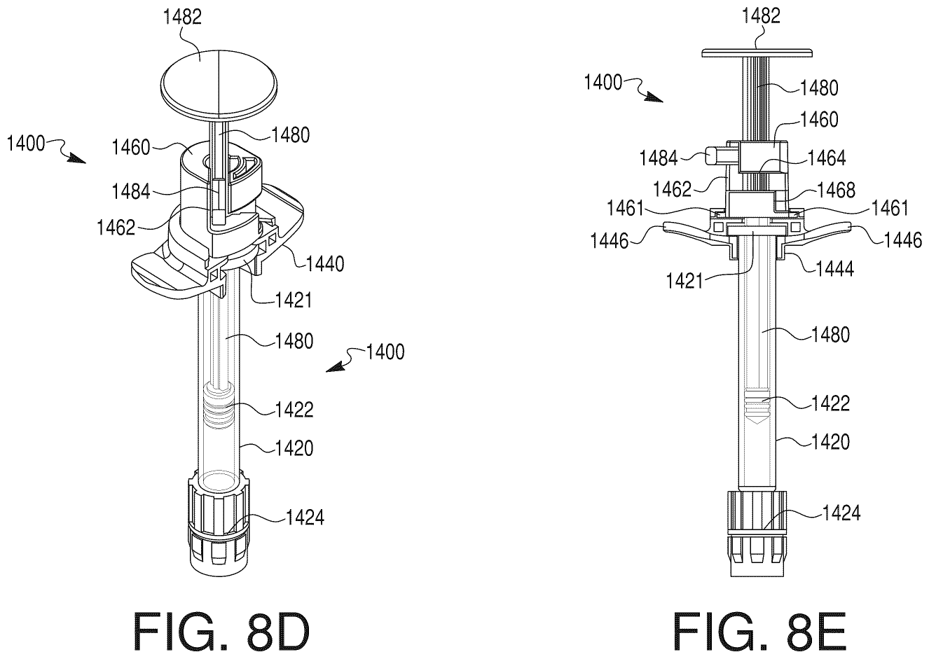

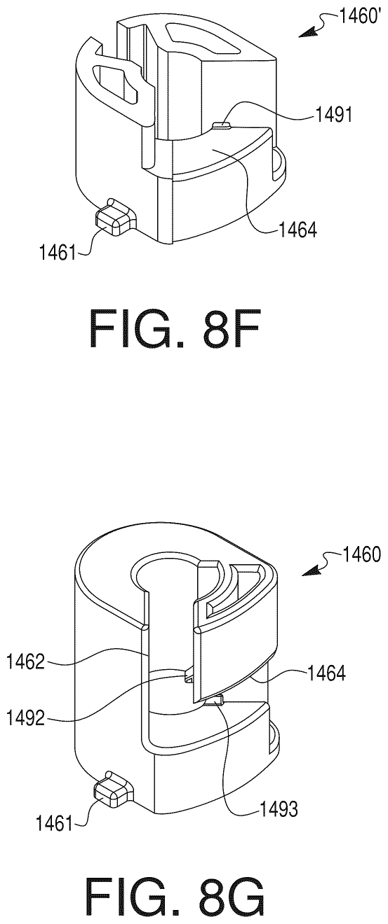

FIGS. 8A-8G depict a further exemplary delivery device 1400 and component parts Delivery device 1400 includes a plunger rod 1480, a blocking component 1460, a flange piece 1440, and a body 1420. Plunger rod 1480 includes an actuation portion 1482 and a protrusion 1484. Blocking component 1460 may be a rotatable alignment component that is configured to partially or fully surround plunger rod 1480, and includes three connected channels 1462, 1464, 1468 sized and configured to allow for passage of protrusion 1484. Flange piece 1440 includes a proximal collar 1442 having a channel 1447 into which tabs 1461 of blocking component 1460 may slidably fit, a distal collar 1444 including a channel 1445 into which a flange 1421 of body 1420 may fit (e.g., may be slidably assembled), and flanges 1446.

Body 1420 (depicted in FIGS. 8D and 8E) may have any or all of the same characteristics as, e.g., body 1060 of device 1050, or as any syringe body known in the art. For example, in some embodiments, body 1420 may be pre-fillable or pre-filled. A stopper 1422 may be configured to be inserted into body 1420 and may be configured to hold a predetermined volume of a formulated drug substance inside body 1420, between stopper 1422 and an expulsion end 1424.

Delivery device 1400 may be, for example, an injection device, such as a syringe, for dispensing a predetermined volume of a formulated drug substance. Generally, delivery device 1400 may share size, capacity, material, preparation, assembly, or manufacturing characteristics with device 1050, device 1200, device 1300, or with other delivery devices disclosed herein. As with other devices disclosed herein, delivery device 1400 may be configured for ease of use and may include one or more features that aid a user by providing tactile, auditory, or visual feedback, using any of the features described elsewhere herein.

Blocking component 1460 may be of any suitable size and/or shape to assist in controlling proximal and distal movement of plunger rod 1480 in device 1400.

Flange piece 1440 may be of any suitable size and shape to close, partially close, cover, or partially cover an end of body 1420 opposite expulsion end 1424, and/or to support and hold blocking component 1460 and plunger rod 1480 in relation to body 1420. For example, proximal collar 1442 and channel 1447 may be sized and configured to hold blocking component 1460, and distal collar 1444 and channel 1445 may be sized and configured to hold a flange 1421 of body 1420, such that blocking component 1460 is held stationary in relation to body 1420. Further, blocking component 1460 may be sized and configured to plunger rod 1480 inside body 1420, and to limit movement of plunger rod 1480 with respect to body 1420. Flange piece 1440 may include one or more flanges 1446, which may be sized and configured to aid a user in holding device 1400 and/or expelling a formulated drug substance from expulsion end 1424. In some embodiments, as depicted in FIGS. 8A-8E, flange piece 1440 may include two flanges 1446, opposite to one another. In general, other arrangements of a flange or flanges, such as one flange or three flanges, are possible. Flange piece 1440 may extend radially outward from a central longitudinal axis of device 1400 farther than a circumference of body 1420, to, e.g., support device 1400 if device 1400 is placed on a surface, prevent device 1400 from rolling on a flat surface, and/or allow device 1400 to be picked up more easily.

Channels 1462, 1464, 1468 in blocking component 1460 together form a path through which protrusion 1484 may travel, to allow for controlled movement of plunger rod 1480. A first channel 1462 may allow for sufficient distal movement of plunger rod 1480 to prime device 1400. A second channel 1464 may allow for movement of the plunger rod between a “primed” state and a “delivery” state. Channel 1464 may have a path requiring rotation of plunger rod 1480 about a longitudinal axis of device 1400 (as opposed to distal movement of plunger rod 1480), such that the likelihood of plunger rod 1480 being accidentally or unintentionally moved to a “delivery” state may be reduced. Channel 1464 may provide a path of any suitable length (corresponding to any suitable angle of rotation of plunger rod 1480) to ensure adequate separation between the “primed” state and the “delivery” state. A third channel 1468 may allow for sufficient distal movement of plunger rod 1480 to dispense a predetermined volume of drug substance from device 1400.

One or more of each channel 1462, 1464, 1468 may include one or more detents, as shown in FIGS. 8F and 8G. For example, a cross sectional view of blocking component 1460 in FIG. 8F shows an interior of channel 1464 having a small detent 1491 disposed on one side. FIG. 8G depicts two larger detents 1492, 1493 in channels 1462, 1464, respectively. Each detent may provide resistance to the movement of protrusion 1484 through channels 1462, 1464, and/or 1468 to provide auditory feedback and/or to prevent unintended movement of protrusion 1484. In some embodiments, detents 1491, 1492, 1493 may be angled on one side, to allow for passage of protrusions 1484 in one direction, but not in the other direction. Detents such as those shown in FIGS. 8F and 8G may be suitable for inclusion in any device disclosed herein, as well as in device 1400.

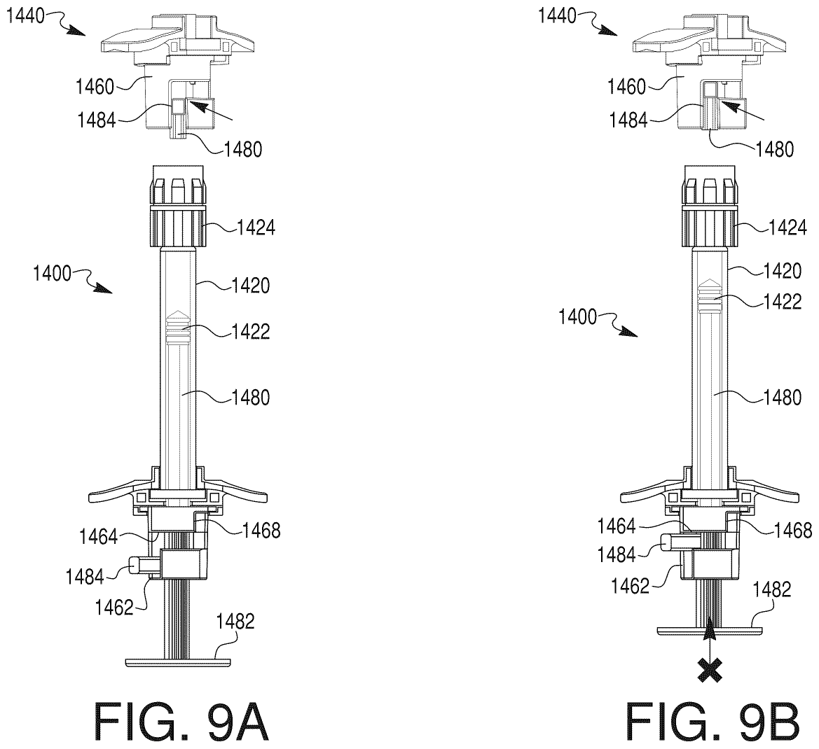

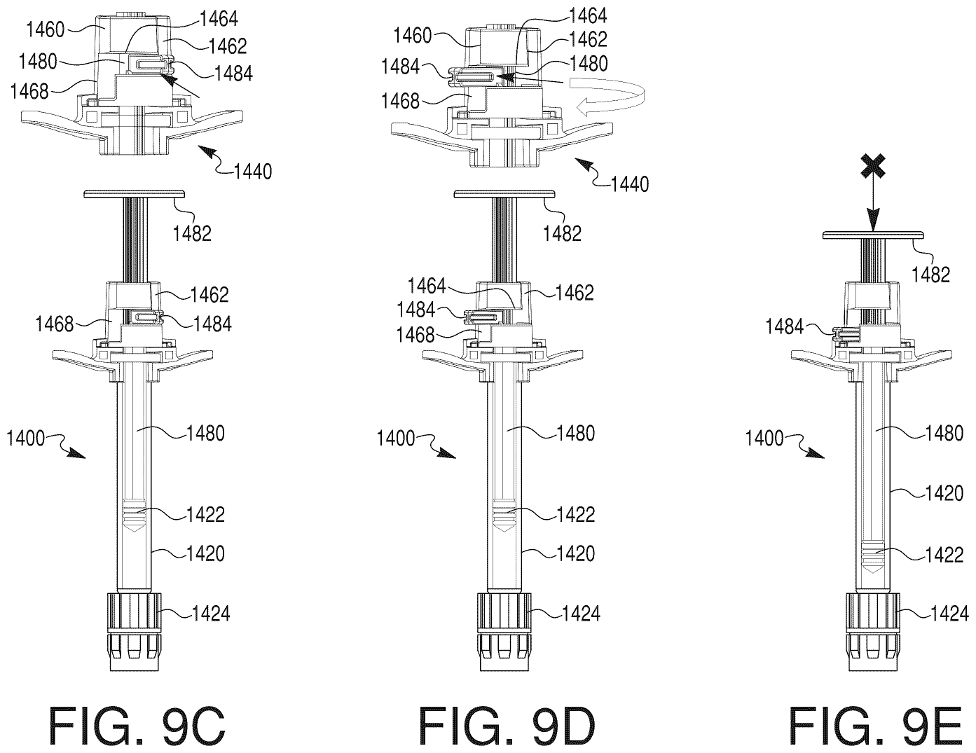

FIGS. 9A-9E depict an exemplary method of using device 1400, according to aspects of the present disclosure. In a pre-use configuration depicted in FIG. 9A, device 1400 may hold a volume of a drug substance in between stopper 1422 and expulsion end 1424. Plunger rod 1480 may be partially inserted into body 1420 such that protrusion 1484 of plunger rod 1480 is disposed in a proximal end portion of channel 1462. In a priming step depicted in FIG. 9B, plunger rod 1480 may be moved longitudinally further into body 1420, until distal movement is blocked by the abutment of protrusion 1484 against a distal end of channel 1462. For example, a user may press actuation portion 1482 distally through blocking component 1460. In some embodiments, device 1400 may be held in an inverted position during this step, to ensure that air trapped in body 1420 may be expelled, as stopper 1422 is pushed distally by plunger rod 1480. In the “primed” configuration, depicted in FIG. 9C, protrusion 1484 of plunger rod 1480 may be disposed at a first end of channel 1464.

In a dispensing preparation step depicted in FIG. 9D, plunger rod 1480 may be rotated about a longitudinal axis such that protrusion 1484 is moved from a first end of channel 1464 to a second end of channel 1464. For example, a user may grasp and twist actuation portion 1482 of plunger rod 1480. Device 1400 may then be in a ready-to-dispense configuration, wherein protrusion 1484 is disposed at a proximal end of channel 1468. As depicted in FIG. 9E, in a dispensing step, plunger rod 1480 may be moved longitudinally further into body 1420. For example, a user may press actuation portion 1482 distally, until protrusion 1484 abuts a distal end of channel 1468. The dispensing step may ensure that a predetermined volume of a drug substance inside body 1420 is dispensed from device 1400.

In some embodiments, after each successive step in the use of device 1050, a user may be prevented from re-doing a step, and/or from reversing one or more steps. For example, geometries of, e.g., plunger rod 1480, protrusion 1484, and/or channels 1462, 1464, 1468 may prevent a user from pulling plunger rod 1480 proximally (e.g., out of) body 1420.

FIGS. 10A-10C depict an exemplary method of assembly of device 1400. As depicted in FIG. 10A, flange piece 1440 may be slidably assembled to body 1420 such that flange 1421 fits into channel 1445 and collar 1444 partially surrounds body 1420. As depicted in FIG. 10B, blocking component 1460 may be slidably assembled to flange piece 1440, such that tabs 1461 rest within channels 1447 and blocking component 1460 abuts proximal collar 1442. As depicted in FIG. 10C, plunger rod 1480 may then be inserted into the combined blocking component 1460, flange piece 1440, and body 1420, such that protrusion 1484 is disposed within channel 1462 of body 1420.

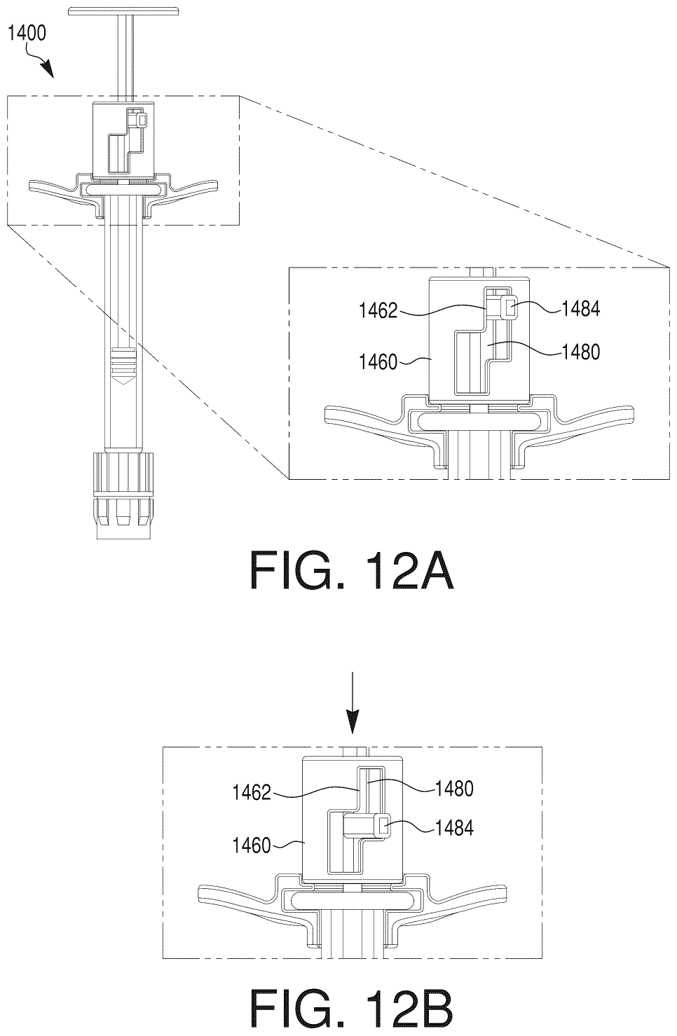

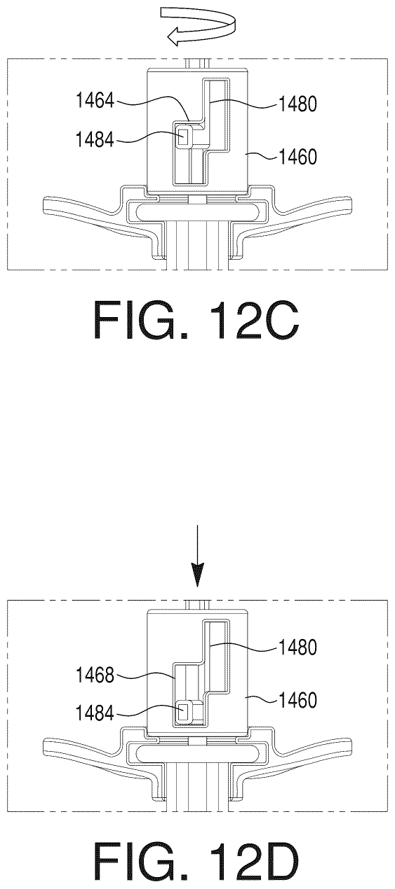

FIGS. 10D-10G, 11A-11E, and 12A-12D depict a variation on a configuration and method of use of device 1400, and to avoid redundancy will not be described in great detail. FIGS. 10D-10G depict an alternate method of assembly of device 1400, where blocking component 1460 includes an opening 1463 through which plunger rod 1480 may fit. In this embodiment, the channels within blocking component 1460 (e.g., channels 1462, 1468) may be closed on a proximal and distal end, to prevent back-out or over-insertion of plunger rod 1480 relative to body 1420. As depicted in FIG. 10E, plunger rod 1480 may be partially inserted into body 1420, and flange piece 1440 may be slidably assembled to body 1420 such that flange 1421 fits into channel 1445 and collar 1444 partially surrounds body 1420. As depicted in FIG. 10F, blocking component 1460 may be assembled to plunger rod 1480, such that protrusion 1484 is disposed within one of the channels in blocking component 1460. As depicted in FIG. 10G, blocking component 1460 may then be assembled to flange piece 1440 such that it is disposed in channel 1447. Blocking component may be affixed to flange piece 1440 in any suitable manner (e.g., using clips, adhesive, a friction fit, a dovetail connection, etc.). FIGS. 12A-12D depict a close-up view of protrusion 1484 moving through the channels of blocking component 1460, per the method of use shown in FIGS. 11A-11E.

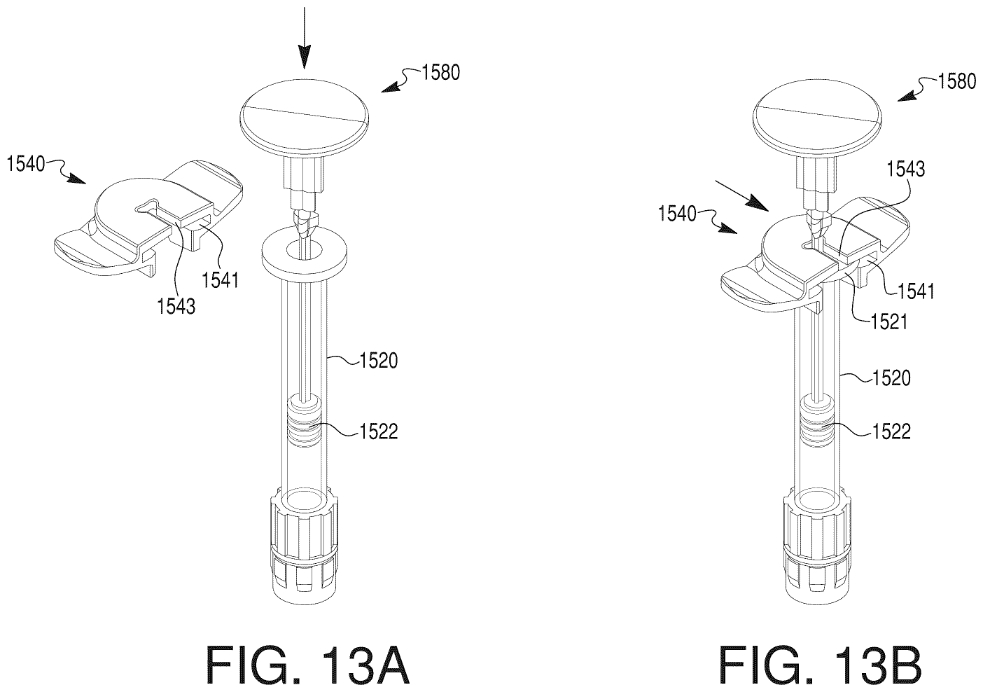

FIGS. 13A and 13B depict a further exemplary delivery device 1500, and a method of assembling said delivery device, according to additional embodiments of the present disclosure. Device 1500 includes a plunger rod 1580, a blocking component in the form of flange piece 1540, and a body 1520. To assemble device 1500, plunger rod may be inserted into body 1520 (e.g., as shown in FIG. 13A), such that it abuts or attaches to a stopper 1522 in body 1520, and flange piece 1540 may be slidably assembled to 1521, e.g., by sliding a channel 1541 on to a flange 1521 of body 1520 (e.g., as shown in FIG. 13B). An opening 1543 may allow for flange piece 1540 to be assembled to body 1520 around plunger rod 1580. It is contemplated that, depending on the size, shape, and structure of each component of device 1500, alternate methods of assembly are possible.

Delivery device 1500 may be, for example, an injection device, such as a syringe, for dispensing a predetermined volume of a formulated drug substance. Generally, delivery device 1500 may share size, capacity, material, preparation, assembly, or manufacturing characteristics with device 1050, device 1200, device 1300, or with other delivery devices disclosed herein. As with other devices disclosed herein, delivery device 1500 may be configured for ease of use and may include one or more features that aid a user by providing tactile, auditory, or visual feedback, using any of the features described elsewhere herein.

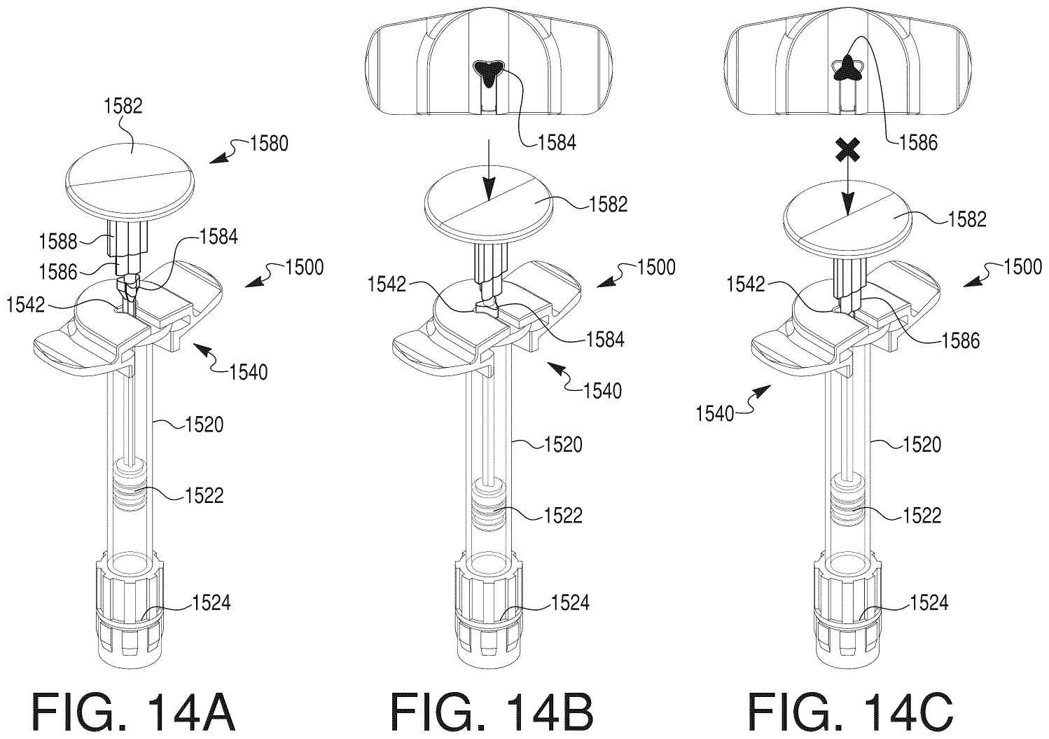

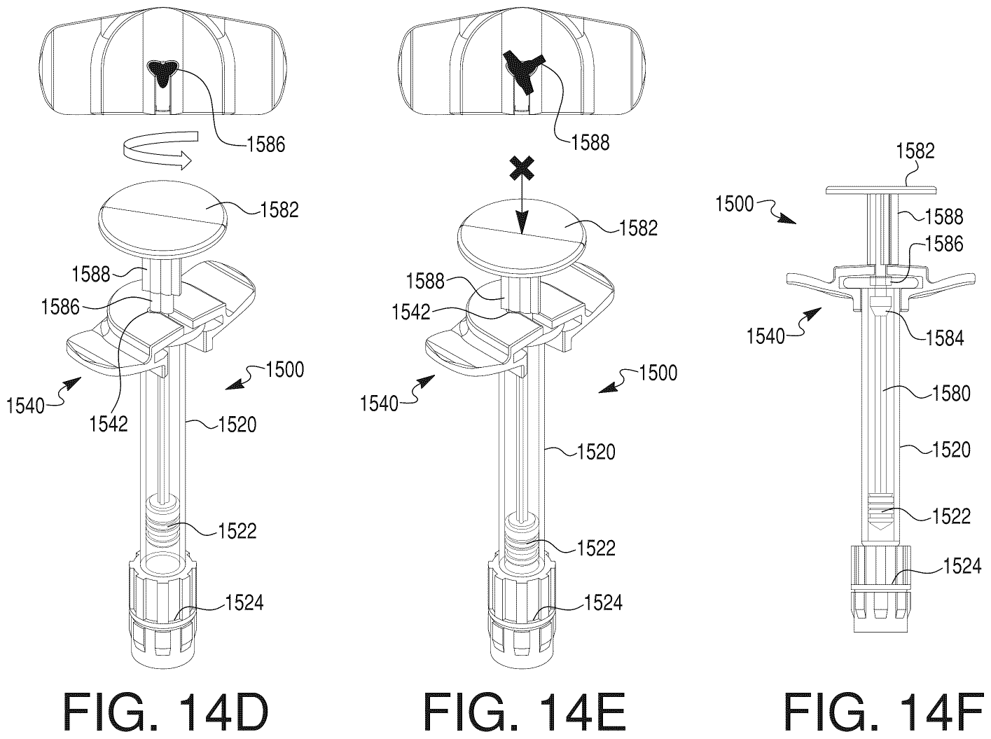

FIG. 14A-14F depict a further view of device 1500 and a method of using device 1500. As shown in FIG. 14A, plunger rod 1580 may include an actuation portion 1582, a proximal stop 1588, a proximal neck portion 1586, and a distal neck portion 1584. Body 1520 may have any or all of the same characteristics as, e.g., body 1060 of device 1050, or as any syringe body known in the art. For example, in some embodiments, body 1520 may be pre-fillable or pre-filled. Stopper 1522 may be configured to be inserted into body 1520 and may be configured to hold a predetermined volume of a formulated drug substance inside body 1520, between stopper 1522 and an expulsion end 1524.

Flange piece 1540 may be of any suitable size and shape to partially close, cover, or partially cover an end of body 1520 opposite expulsion end 1524, and/or to support and hold plunger rod 1580 in body 1520. An opening 1542 may have a size and shape configured to allow passage of plunger rod 1580 in two different configurations. Distal neck portion 1584 and proximal neck portion 1586 may have similar shapes, but may be rotationally offset from one another (e.g., such that once distal neck portion 1584 passes through opening 1542, plunger rod 1580 must be rotated about a longitudinal axis to allow proximal neck portion 1587 to pass. Distal neck portion 1584 may include, e.g., a tapered distal side, which may assist in orienting plunger rod 1580 such that distal neck portion 1584 may pass through opening 1542. This may increase the ease of, e.g., a priming step.

FIG. 14A depicts a pre-use configuration of device 1500. In such a configuration, device 1500 may hold a volume of a drug substance in between stopper 1522 and expulsion end 1524. Plunger rod 1580 may be partially inserted into body 1520 such that distal neck portion 1584 is positioned proximally from flange piece 1540. In a priming step depicted in FIG. 14B, plunger rod 1580 may be moved longitudinally further into body 1520, until distal movement is blocked by the abutment of proximal neck portion 1586 against opening 1542 (as shown in FIG. 14C). For example, a user may press actuation portion 1582 until distal neck portion passes through opening 1542. In some embodiments, device 1500 may be held in an inverted position during this step, to ensure that air trapped in body 1520 may be expelled, as stopper 1522 is pushed distally by plunger rod 1580. In the “primed” state, depicted in FIG. 14D, proximal neck portion 1586 may be disposed against a surface of flange piece 1540.

In a dispensing preparation step depicted in FIG. 14D, plunger rod 1580 may be rotated about a longitudinal axis such that the shape of proximal neck portion 1586 aligns with opening 1542. For example, a user may grasp and twist actuation portion 1582 of plunger rod 1580. Device 1500 may then be in a ready-to-dispense configuration. As depicted in FIG. 14E, in a dispensing step, plunger rod 1580 may be moved longitudinally further into body 1520. For example, a user may press actuation portion 1582 distally, until proximal stop 1588 abuts a surface of flange piece 1540. The dispensing step may ensure that a predetermined volume of a drug substance inside body 1520 is dispensed from device 1500.

In some embodiments, after each successive step in the use of device 1500, a user may be prevented from re-doing a step, and/or from reversing one or more steps. For example, geometries of, e.g., plunger rod 1580, distal neck portion 1584, proximal neck portion 1586, and opening 1542 may interface with one another to prevent a user from pulling plunger rod 1580 proximally (e.g., out of) body 1520.

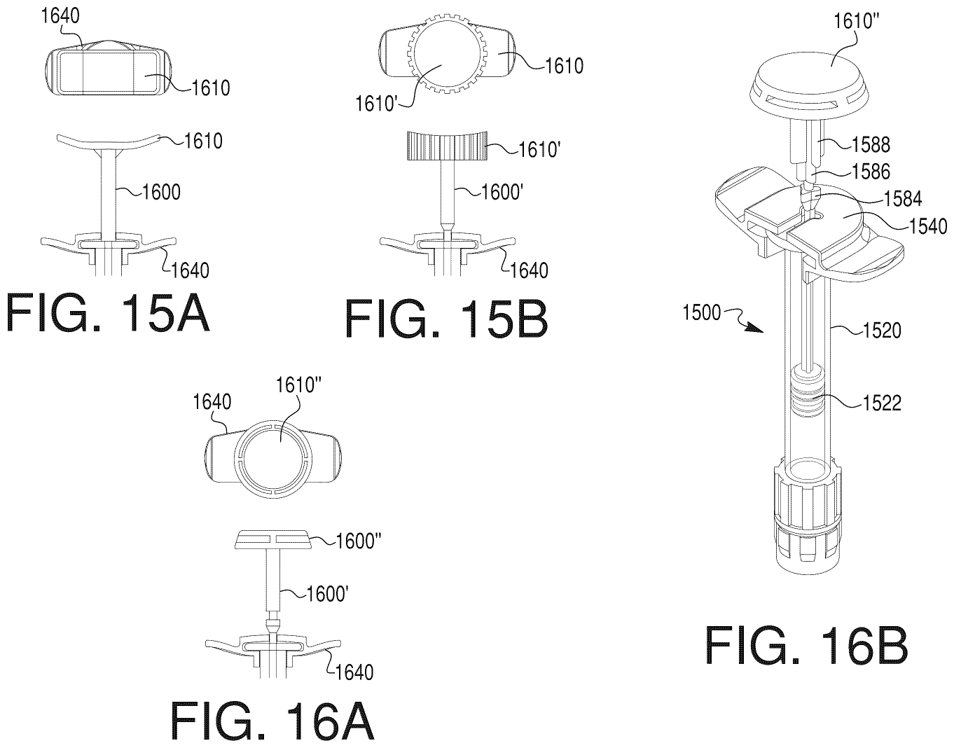

Additional variations on blocking components, dosage control components, and the like will now be described. FIGS. 15A-23C depict exemplary plunger rod dials according to further embodiments of the present disclosure. For example, FIG. 15A depicts a plunger rod 1600 having an actuation portion 1610. Actuation portion 1610 may have a shape generally corresponding to a flange piece 1640. Plunger rod 1600 may be rotatable with respect to flange piece 1640 and/or a body of the device. A device may be in a configuration suitable for delivery of a desired amount of a drug substance when, e.g., a shape of plunger rod 1600 is generally aligned with shape of 1640 (as shown in, e.g., the top view of FIG. 15A). As another example, FIG. 15B depicts an actuation portion 1610′ with a ridged side, to allow for ease of rotation of plunger rod 1600′ with respect to flange piece 1640 and/or a remainder of the syringe. FIG. 16A depicts an actuation portion 1610″ with a ribbed side, again to allow for ease of rotation of plunger rod 1600. FIG. 16B depicts an exemplary combination of actuation portion 1610″ with device 1500. One of ordinary skill in the art will understand that any of the actuation portions or other features described herein may be combined with devices described herein.

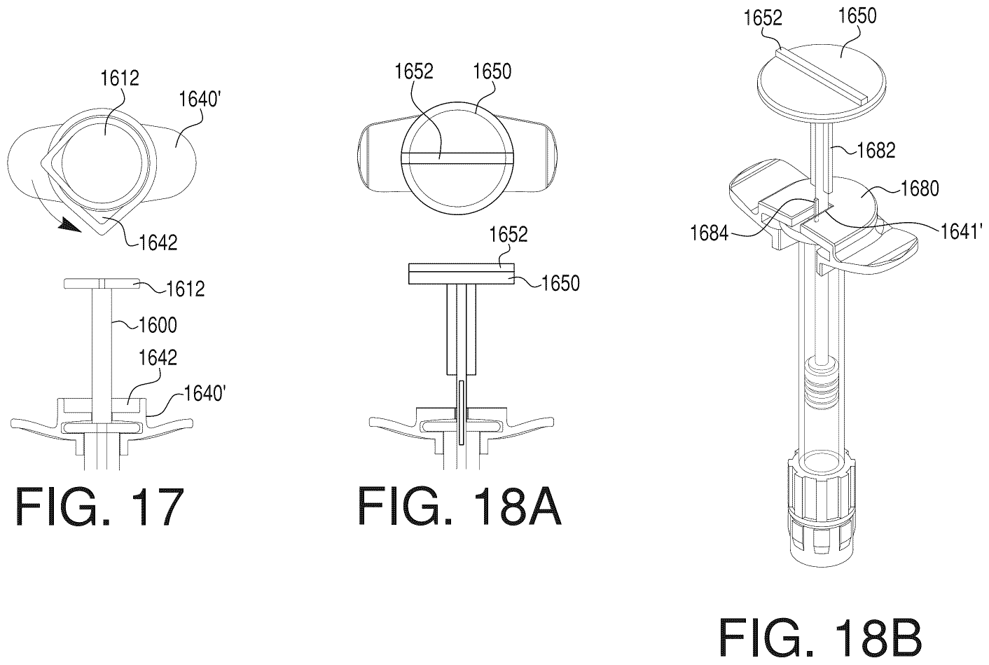

FIG. 17 depicts an exemplary plunger rod and dial according to further embodiments of the present disclosure. An actuation portion 1612 may be sized and configured to fit into a collar 1642 of a flange piece 1640′ in only a particular configuration. A depth of collar 1642 may correspond to, e.g., a distance that plunger rod 1600 must travel to dispense a predetermined volume of a drug substance from a drug delivery device. In one embodiment, actuation portion 1612 may be moved distally until it abuts collar 1642, and then may be rotated until its shape corresponds with the shape of collar 1642 so that it may be pushed into collar 1642 in a dispensing step. FIGS. 18A and 18B depict a further exemplary plunger rod and dial, which combine exemplary features that allow for precision dose delivery. The plunger rod may include, e.g., protrusions 1684 and 1682, which may each fit through an opening 1641′ in a flange piece 1680 in a particular configuration. Each of protrusions 1682 and 1684 may correspond to a distance required to deliver a desired volume of a drug substance from a device and/or prime the device. Actuation portion 1650 may include a raised portion 1652, which may aid a user in twisting the plunger rod in relation to flange piece 1680.

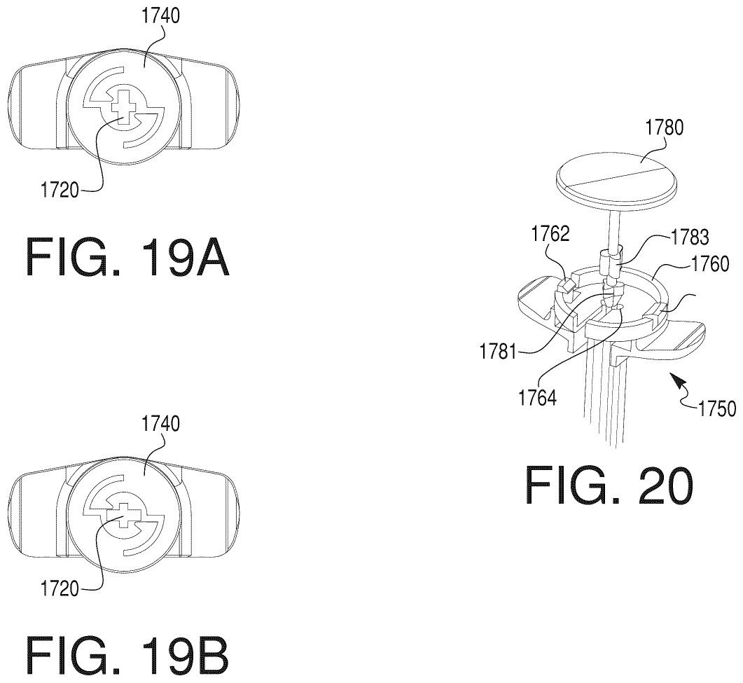

FIGS. 19A and 19B depict a top view of a flange piece 1740 and a plunger rod 1720. Flange piece 1740 and plunger rod 1720 may have a cross-sectional shape allowing for limited rotation of plunger rod 1720 relative to flange piece 1740 in a single direction. For example, flange piece 1740 may have inner protrusions that may interact with an irregular cross-sectional shape of plunger rod 1720 to resist a first portion of plunger rod 1720 as it rotates past the inner protrusions, and to stop a second portion of plunger rod 1720 when it abuts the inner protrusions.

FIG. 20 depicts an exemplary flange piece 1750 with a well 1760 having clips 1762. A plunger rod actuation portion 1780 may be pushed distally into well 1760 until clips 1762 overlay actuation portion 1780, to hold actuation portion 1780 in place and, e.g., prevent back-out of the plunger rod. The plunger rod includes a distal protrusion 1781 and a proximal protrusion 1783, each of which is sized to fit through an opening 1764 when the plunger rod is rotated to a particular position. Distal protrusion 1781 includes a tapered distal side, which may assist in orienting the plunger rod into the position required to advance the plunger rod distally such that distal protrusion 1781 passes through opening 1764. This may increase the ease of, e.g., a priming step. In some embodiments, a height of well 1760 and/or actuation portion 1780 may correspond to a height that a plunger rod must travel to dispense a predetermined volume of a drug substance. Thus, a device may be primed when actuation portion 1780 abuts a proximal side of well 1760, and may deliver a predetermined volume of a drug substance as actuation portion 1780 travels distally into well 1760.

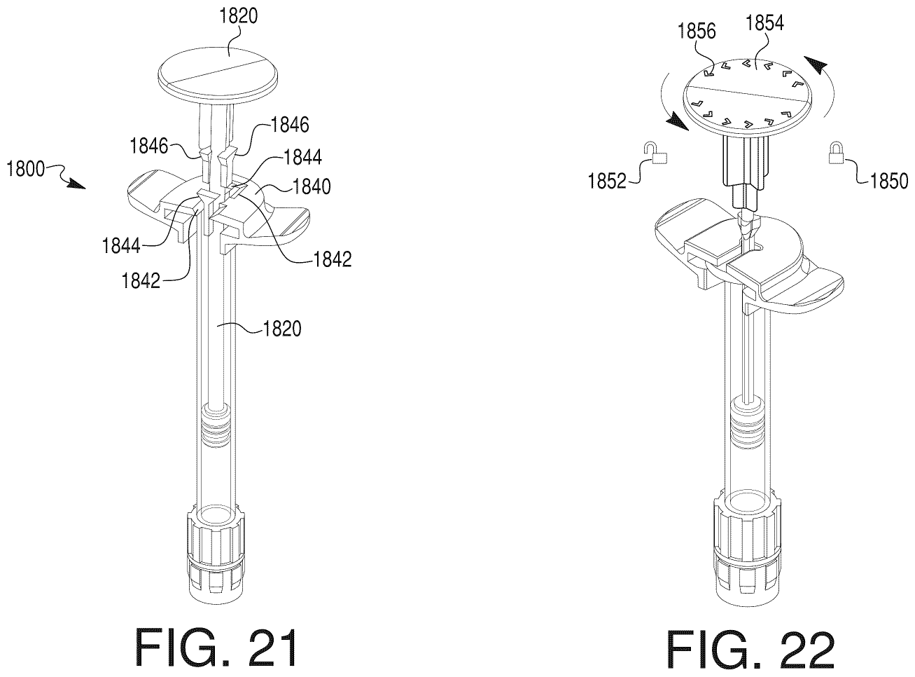

FIG. 21 depicts an exemplary device 1800 with a plunger rod 1820 and a complementary flange piece 1840. Plunger rod 1820 may include, e.g., protrusions 1844, 1846 having an angled or wedge shape, corresponding to a shape of one or more openings 1842 in flange piece 1840. The wedge or angled shapes of protrusions 1844, 1846 and openings 1842 may suffice to resist distal movement of plunger rod 1820 when a protrusion 1844 or 1846 abuts a side of opening 1842, but may be able to move past one another given enough force. The resistance provided by the abutment of protrusions 1844, 1846 against the sides of openings 1842 may suffice to indicate to a user that a particular step in the use of device 1800 is completed. A user may then apply enough force to move plunger rod 1820 past the resistance and continue to a next step (e.g., from a completed priming step to a delivery-ready step).

As has been described elsewhere, any of the devices disclosed herein may be combined with labels, auditory feedback, and/or tactical feedback in the form of symbols (e.g., in FIG. 22 depicted as lock and unlock symbols 1850, 1852, chevrons 1856 on actuation portion 1854). Rotation of a plunger rod also may be accompanied by a “clicking” sound.

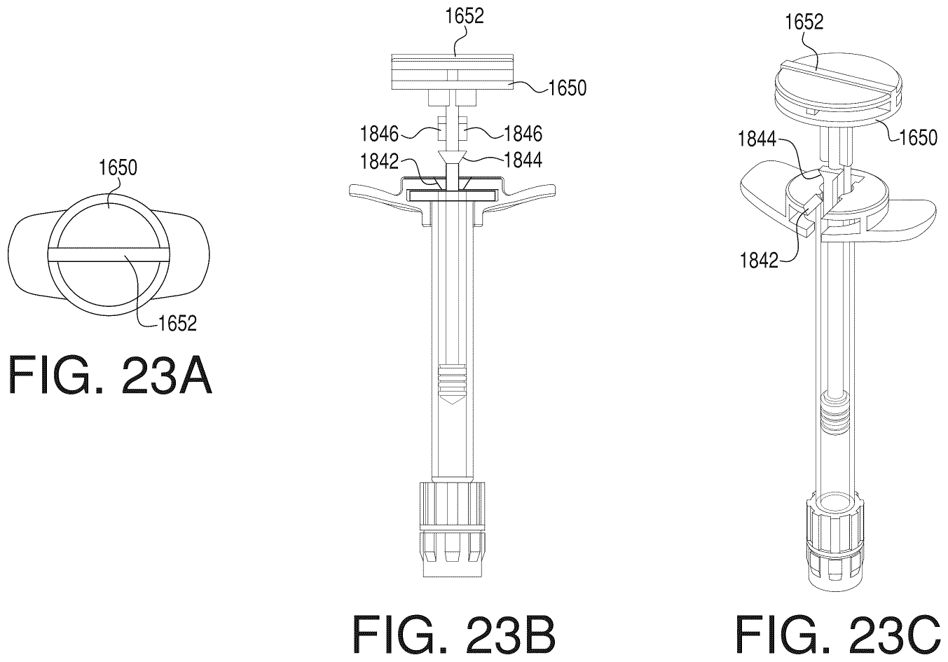

FIGS. 23A-23C depict a further exemplary combination of components in a delivery device. For example, a plunger rod actuation portion 1650 may include, e.g., ribbed sides and a raised portion 1652, to assist in twisting the actuation portion. A device with these characteristics may include, e.g., openings 1842 and corresponding angled protrusions 1844, 1846 (described with respect to FIG. 21).

FIGS. 24A-24E depict a further exemplary delivery device 1900 and a method of using device 1900. Device 1900 may include an actuation portion 1940 and a blocking component 1980 depicted on a plunger rod 1920. Plunger rod 1920 may abut a stopper 1912 in a body 1910. Blocking component 1980 may be rotatable relative to plunger rod 1920. In a pre-use configuration depicted in FIG. 24B, blocking component 1980 may be in a first position with respect to plunger rod 1920 and flange piece 1960. In a priming step depicted in FIG. 24C, plunger rod 1920 may be moved longitudinally further into body 1910, until distal movement is blocked by the abutment of blocking component 1980 against a recess 1962 in flange piece 1960. For example, a user may press actuation portion 1940 distally towards flange piece 1960. In a dispensing preparation step depicted in FIG. 24D, blocking component 1980 may be rotated such that a shorter dimension of blocking component 1980 faces flange piece 1960. Recess 1962 may be curved to allow for ease of rotation of blocking component 1980. A distance between blocking component 1980 and flange piece 1960 after blocking component 1980 is rotated may correspond to a distance that plunger rod 1920 may move to dispense a predetermined volume of a drug substance from device 1900. As depicted in FIG. 24E, in a dispensing step, plunger rod 1920 may be moved longitudinally further into body 1910, until the rotated blocking component 1980 abuts flange piece 1960 in a second position. For example, a user may press actuation portion 1940 distally, until protrusion blocking component abuts flange piece 1960. The dispensing step may ensure that a predetermined volume of a drug substance inside body 1910 is dispensed from device 1900.

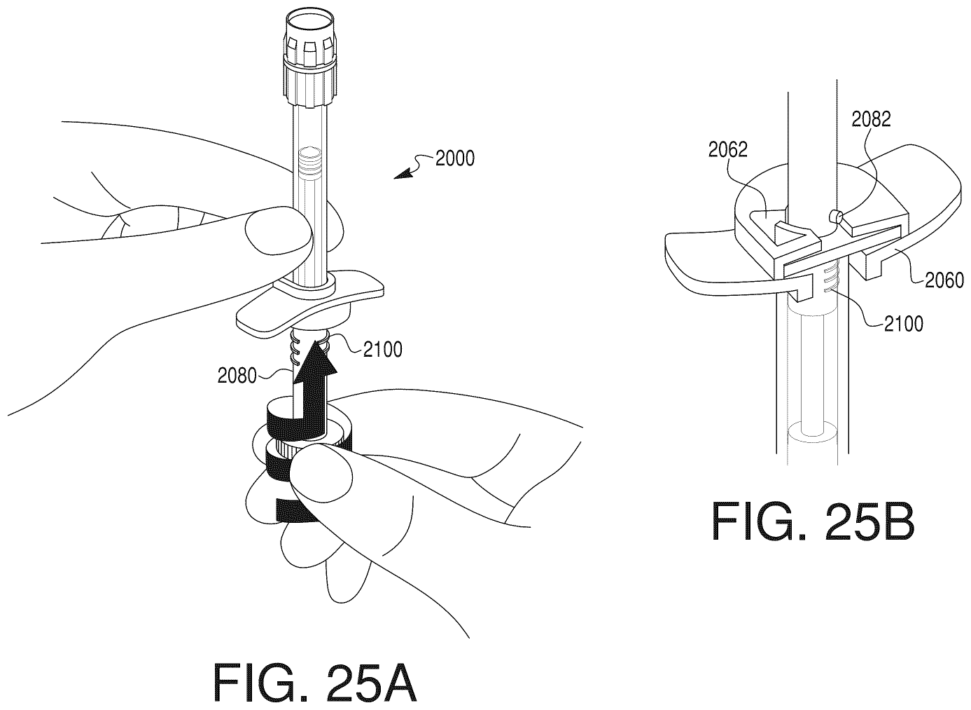

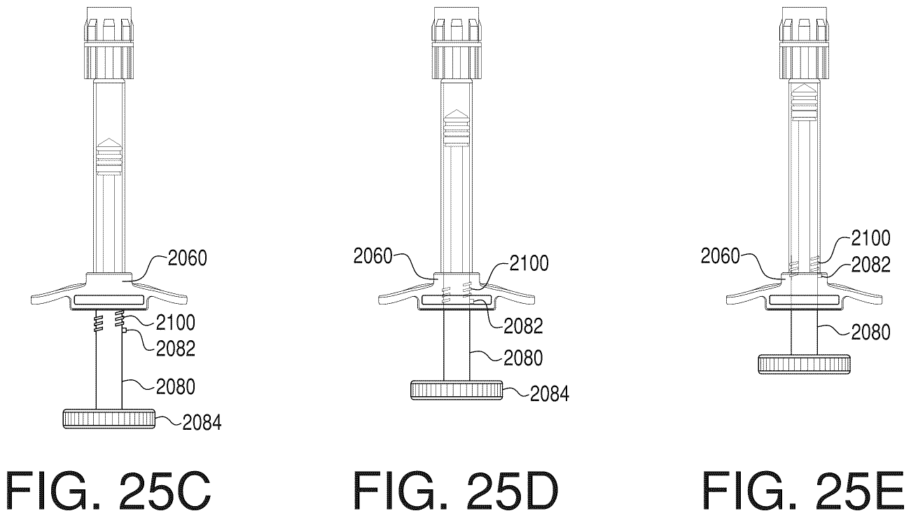

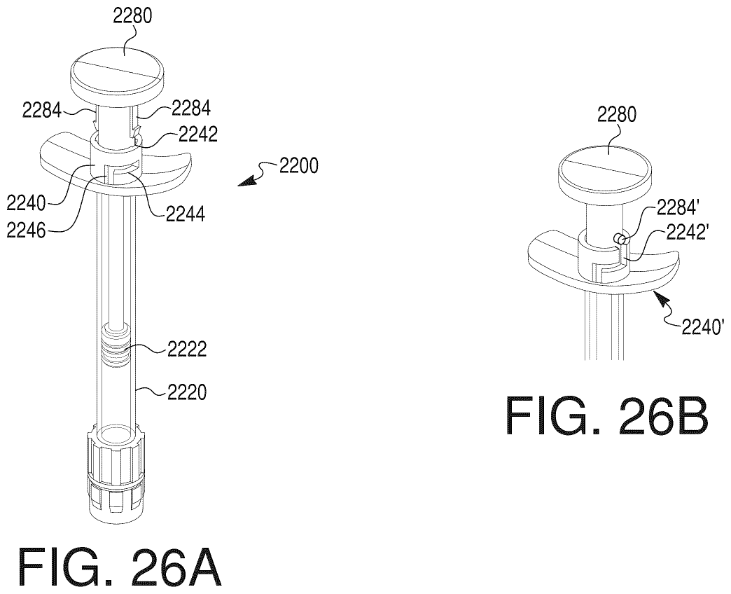

FIGS. 25A-25E depict a further exemplary delivery device 2000, and a method of using delivery device 2000. A plunger rod 2080 of device 2000 may include threads 2100, corresponding to inner threads (not pictured) in a flange piece 2062. As depicted in FIG. 25A, plunger rod 2080 may be rotatable relative to other portions of device 2000. Plunger rod 2080 may also include a protrusion 2082 located proximally from threads 2100 (see, e.g., FIG. 25B), which may correspond to an opening in a flange piece 2062, such that plunger rod 2080 must be in a particular configuration and position to allow protrusion 2082 to pass into and/or through flange piece 2060. In a pre-use configuration depicted in FIG. 25C, threads 2100 and protrusion 2082 may be positioned proximally to flange piece 2060. In a priming step, plunger rod 2080 may be rotated with respect to the inner threads of flange piece 2060 until threads 2100 pass through flange piece 2060 and/or protrusion 2082 prevents further rotation or distal movement of plunger rod 2080. In a dispensing preparation step, protrusion 2082 may be moved towards opening in flange piece 2062. In a dispensing step, protrusion 2082 may be moved through opening in flange piece 2062 to further advance plunger rod 2080, and to dispense a predetermined volume of a drug substance inside the body of device 2000.

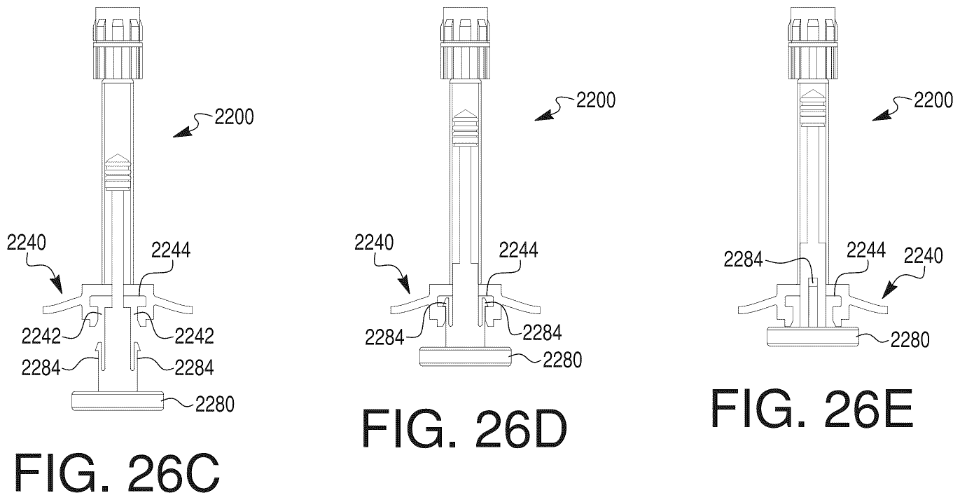

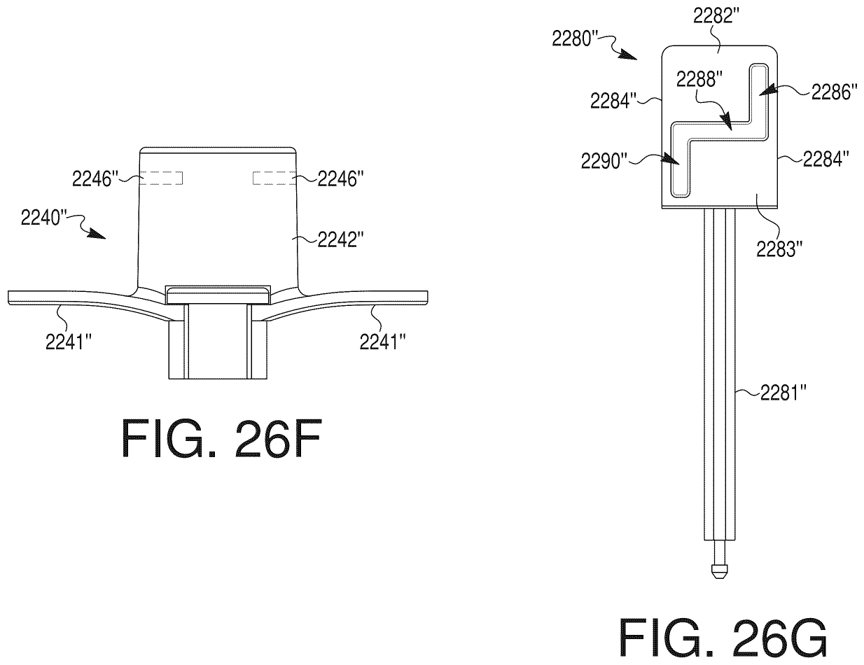

FIGS. 26A-26E depict a delivery device 2200 having further variations on dosage control components. For example, device 2200 includes a plunger rod 2280 with one or more clips 2284, each of which may be configured to slide distally into a channel 2242 of a flange piece 2240 and, once having slid distally, to resist sliding proximally out of channel 2242 (e.g., to prevent or resist back-out of plunger rod 2280). Flange piece 2240 may further have a second channel 2244 and a third channel 2246, through which each of clips 2284 may slide in delivery preparation and dosage delivery steps, as has been previously described. Alternately, as shown in FIG. 26B, channel 2242′ may have an open proximal end through which a protrusion 2284′ may move, allowing for proximal and/or distal movement of a plunger rod 2280 relative to flange piece 2240′. As depicted in FIG. 26C, in a pre-use configuration, clips 2284 may be disposed proximally to channels 2242 of flange piece 2240. In a priming step, plunger rod 2280 may be moved distally into a body of device 2200, until clips 2284 move into channels 2242 and abut a distal end of channels 2242. In a dispensing preparation step, plunger rod 2280 may be rotated relative to flange piece 2240. In a dispensing step, plunger rod 2280 may be moved further distally into a body of device 2200 to dispense a predetermined volume of the drug substance from device 2200.

In other embodiments, as shown in FIGS. 26F-26G, a flange piece 2240″ may include one or more projections 2246″ disposed within a collar 2242″. In the present example, collar 2242″ may include a pair of projections 2246″ extending radially inward from an interior surface of collar 2242″ and in opposite directions relative to another. For example, projections 2246″ may be disposed approximately 180 degrees away from one another. It should be appreciated that flange piece 2240″ may include additional and/or fewer projections 2246″ than those shown and described herein without departing from a scope of this disclosure. Flange piece 2240″ may be configured to engage a plunger rod 2080″ in response to plunger rod 2280″ receiving projections 2246″.

As seen in FIG. 26G, a plunger rod 2280″ may include an actuation member 2284″ defined by a proximal end 2282″ and a distal end 2283″. Plunger rod 2280″ may include a series of channels along opposing sides of actuation member 2284″, such as, for example, a first channel 2286″, a second channel 2288″, and a third channel 2290″ positioned between proximal end 2282″ and distal end 2283″. First channel 2286″ is offset from third channel 2290″ and connected to third channel 2290″ by second channel 2288″ positioned therebetween. As described in detail below, first channel 2286″ may define a longitudinal and axial priming path of plunger rod 2280″, second channel 2288″ may define a circumferential path of plunger rod 2280″, and third channel 2290″ may define a longitudinal and axial dose completion path. It should be appreciated that an opposing surface and/or side of actuation member 2284″ (not shown) includes a substantially similar series of interconnected first channel 2286″, second channel 2288″, and third channel 2290″ as seen in FIG. 26G. In the present example, first channel 2286″ and third channel 2290″ may be aligned parallel relative to one another.

First channels 2286″, second channels 2288″, and third channels 2290″ may be sized, shaped, and configured to receive at least one of the pair of projections 2246″. With plunger rod 2280″ coupled to flange piece 2240″, projections 2246″ may protrude and slide through first channels 2286″, second channels 2288″, and third channels 2290″ to prime and deliver a dosage from device 2200 (FIG. 26A) as described in detail above. In some embodiments, first channels 2286″ may have an open end at proximal end 2282″ through which projections 2246″ may be received in. In some embodiments, first channels 2286″ may have a closed proximal end and projections 2246″ may be at least partially flexible and/or deformable such that projections 2246″ may be configured to flex radially-outward when being received at the proximal end of first channels 2286″. In other embodiments, first channels 2286″ may have a sloped, chamfered, and/or tapered end to facilitate guiding projections 2246″ toward second channels 2288″. In this instance, the sloped end may inhibit retraction (e.g., proximal movement) of plunger rod 2280″ relative to flange piece 2240″. A longitudinal length of first channels 2286″ may define an axial priming path (e.g., an amount or extent priming) that is configured to facilitate proximal and/or distal movement of plunger rod 2280″ relative to flange piece 2240″. For example, projections 2246″ may be disposed at a proximal end of first channels 2286″ and proximally of second channels 2288″ when device 2200 is in an assembly state. In a priming step, plunger rod 2280″ may move distally relative to flange piece 2240″ until projections 2246″ are positioned within second channels 2288″ and at a distal end of first channels 2286″. Second channels 2288″ may define a circumferential path of plunger rod 2280″.

In a dispensing preparation step, plunger rod 2280″ may be rotated relative to flange piece 2240″ to translate projections 2246″ laterally through the circumferential path of second channels 2288″ and toward a dose completion path defined by third channels 2290″. In some embodiments, plunger rod 2280″ and/or flange piece 2240″ may be configured to generate a user feedback (e.g., tactile, audible, visual, etc.) when device 1050 is in the dispensing preparation step. In a dispensing step, plunger rod 2280″ may move distally into a body of device 2200 to dispense a controlled volume of substance by translating projections 2246″ through third channels 2290″. A longitudinal length of third channels 2290″ may define a dosage delivery path (e.g., a dosage amount). It should be appreciated that the axial priming path (length of first channels 2286″) may vary relative to the dosage delivery path (length of third channels 2290″). In other embodiments, plunger rod 2280″ may include additional and/or fewer channels along actuation member 2284″ (e.g., corresponding to a quantity of projections 2246″ on flange piece 2240″), or have various other relative channel configurations, than those shown and described herein.

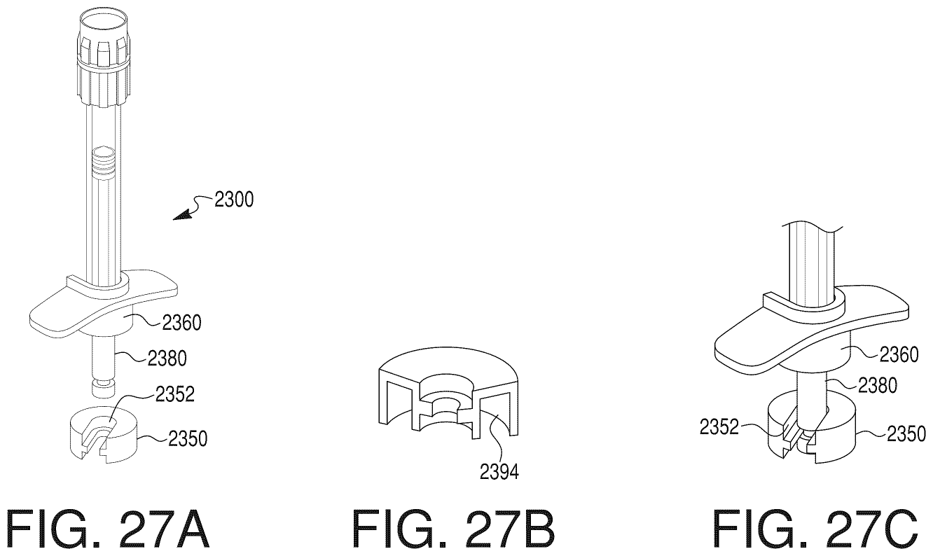

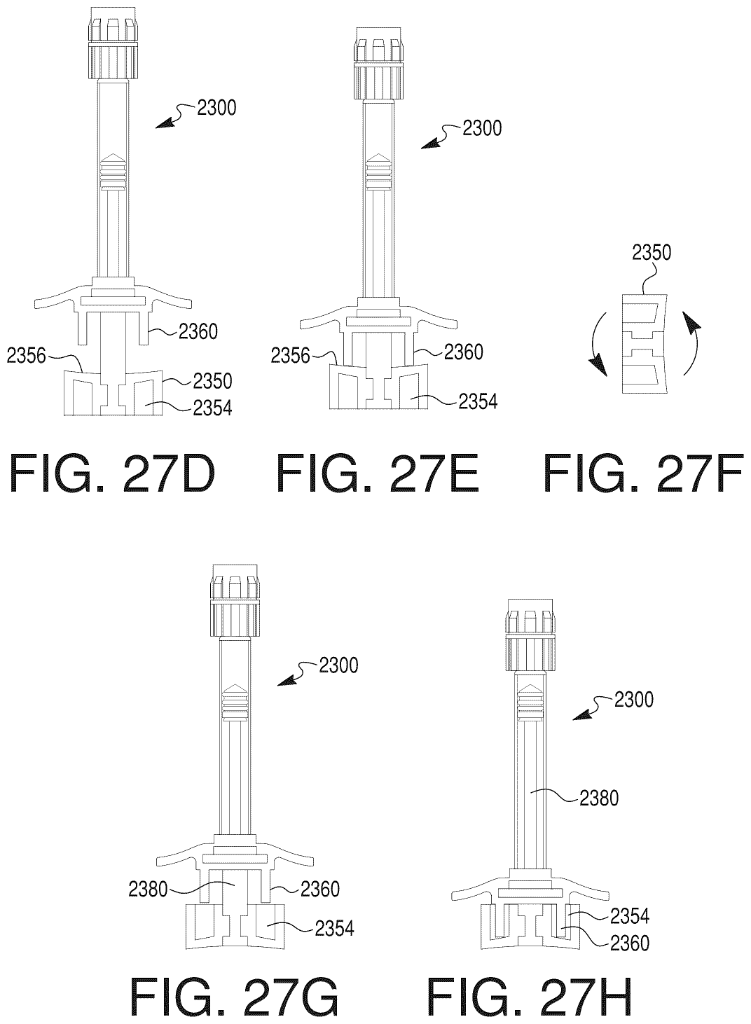

FIGS. 27A-27H depict an exemplary delivery device 2300 and method of using delivery device 2300. An actuation portion 2350 may also serve as a blocking component of device 2300. Actuation portion 2350 may be slidably coupled to plunger rod 2380 in two configurations, via a channel 2352. As depicted in FIG. 27B, one side of actuation portion 2350 may include a channel 2354. A depth of channel 2354 may correspond to a distance that a plunger rod may move to dispense a predetermined volume of a drug substance once device 2300 has been primed. As depicted in FIG. 27C and FIG. 27D, in a pre-use configuration, actuation portion 2350 may be assembled onto plunger rod 2380 such that a flat side of actuation portion 2350 faces a collar 2360 of device 2300. In a priming step, actuation portion 2350 may be used to move plunger rod 2380 distally until the flat side 2356 of actuation portion 2350 abuts a proximal side of collar 2360. To prepare for a dosage delivery step, actuation portion 2350 may be removed from plunger rod 2380, and may be rotated or flipped and reassembled with plunger rod 2380 such that channel 2354 faces collar 2360, as depicted in FIGS. 27F and 27G. In a dosage delivery step, actuation portion 2350 may be used to push plunger rod 2380 further distally, until a proximal end of collar 2360 abuts an inner end of channel 2354. This movement of plunger rod 2380 may be sufficient to dispense a predetermined dose of a drug substance from device 2300.

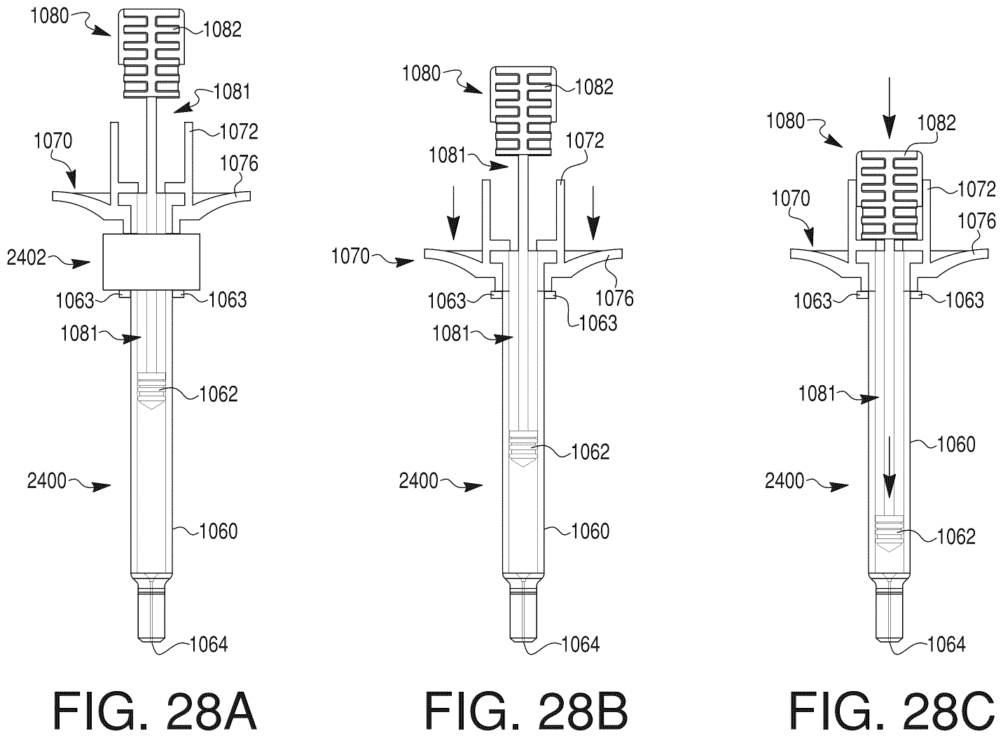

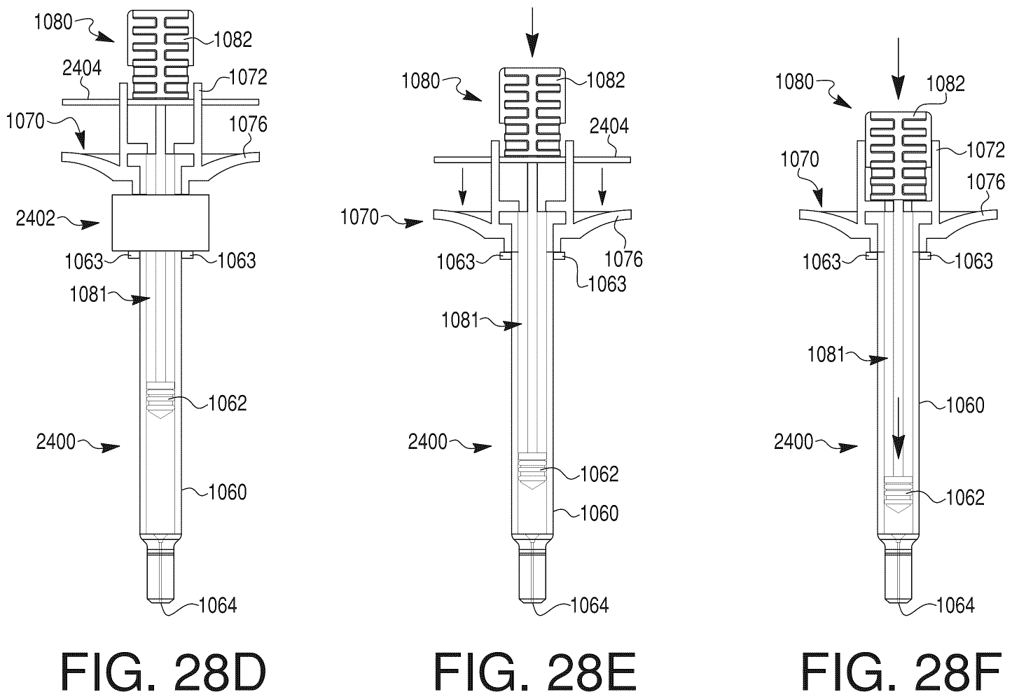

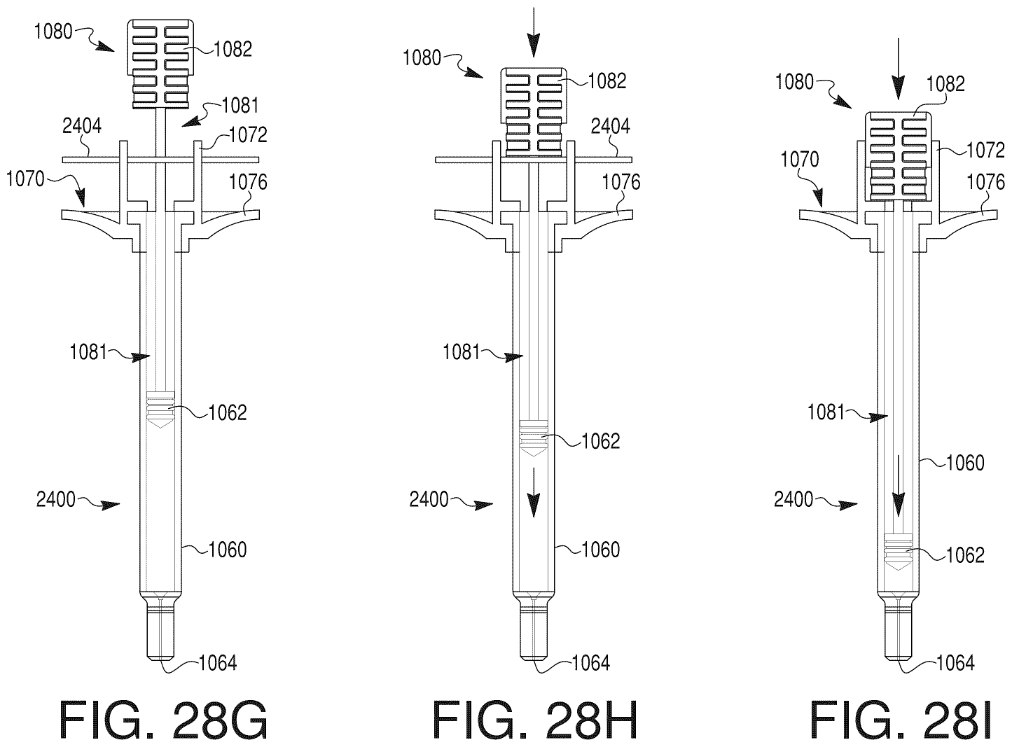

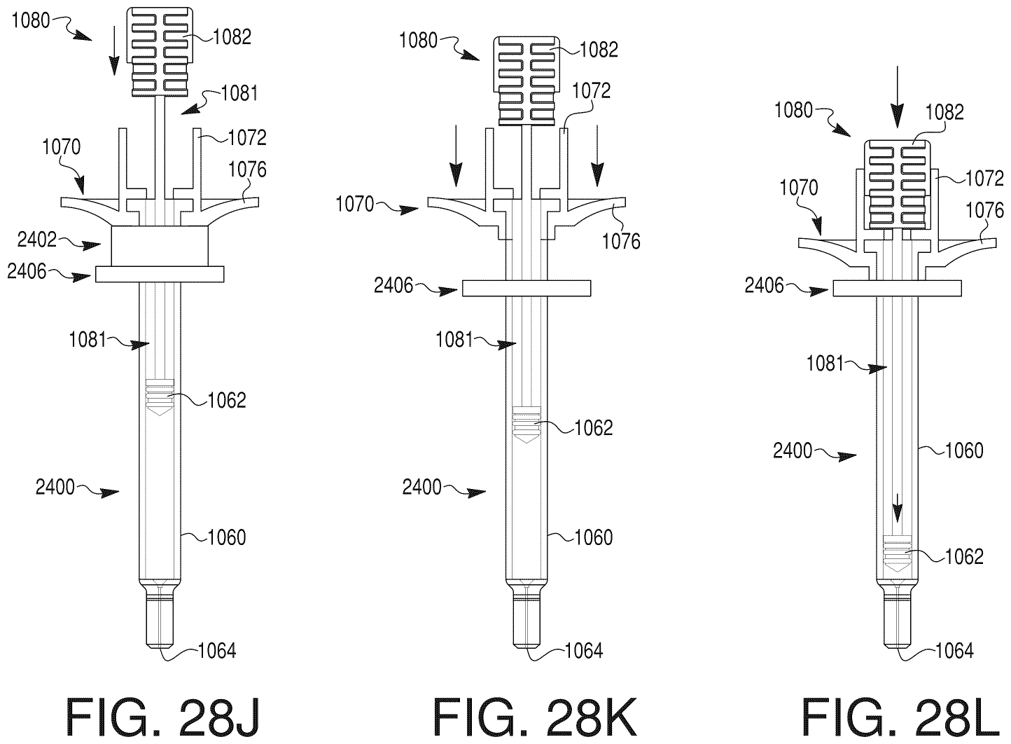

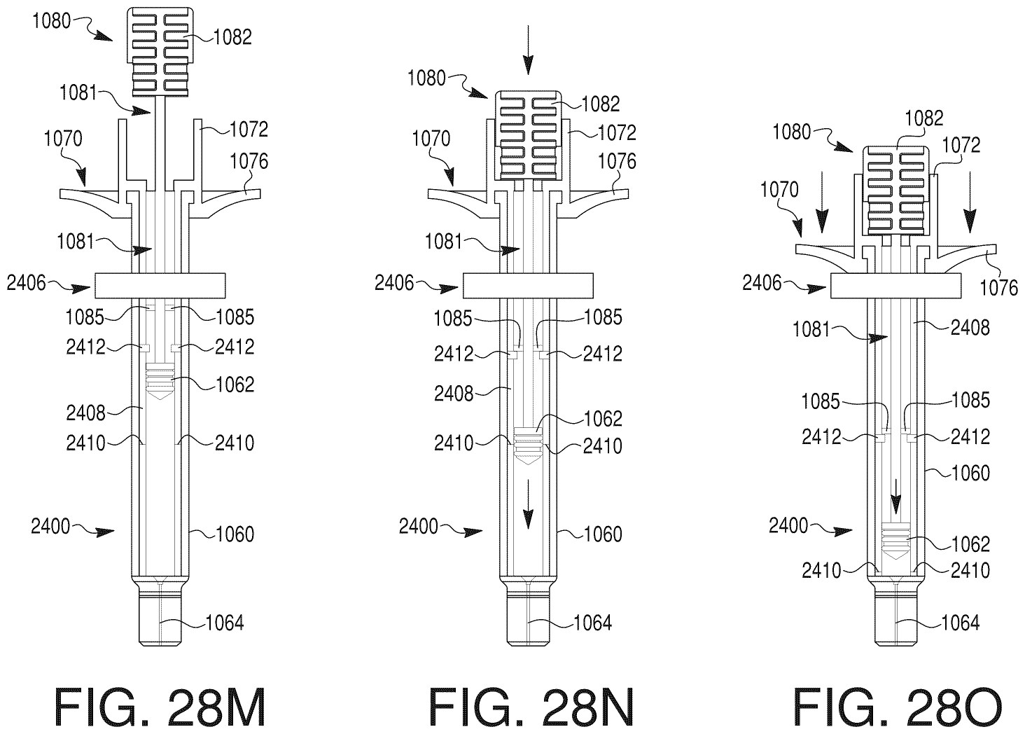

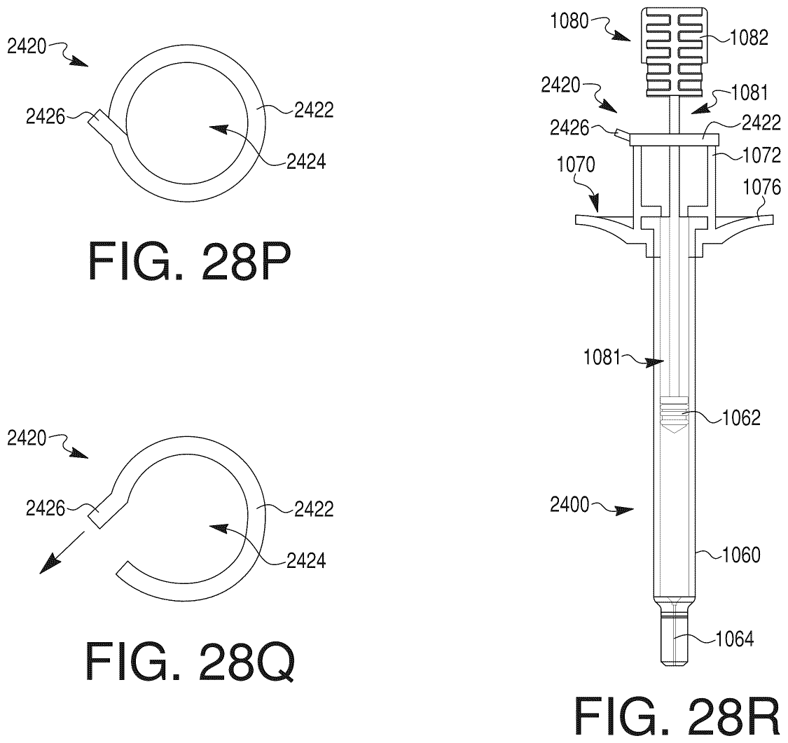

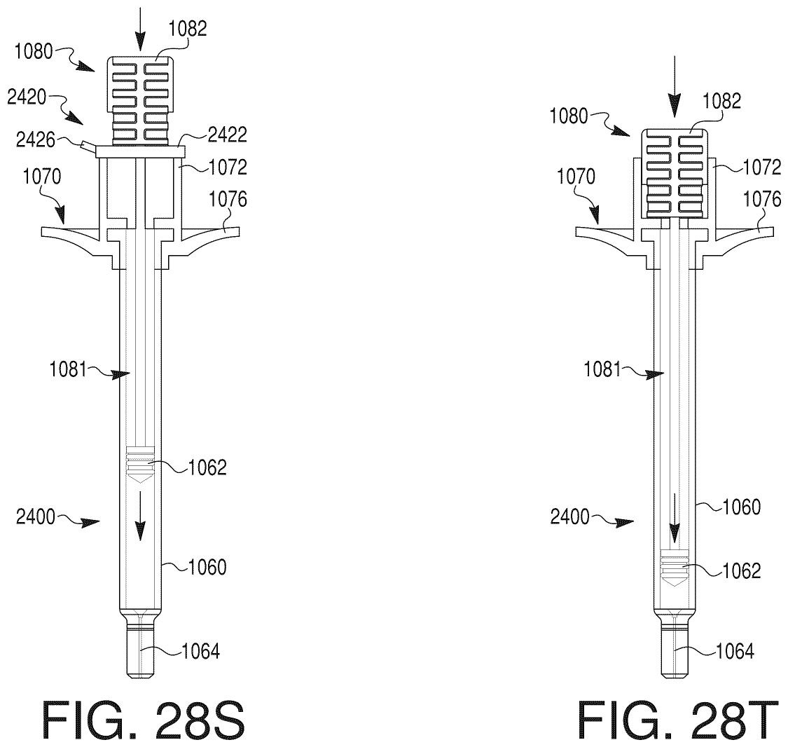

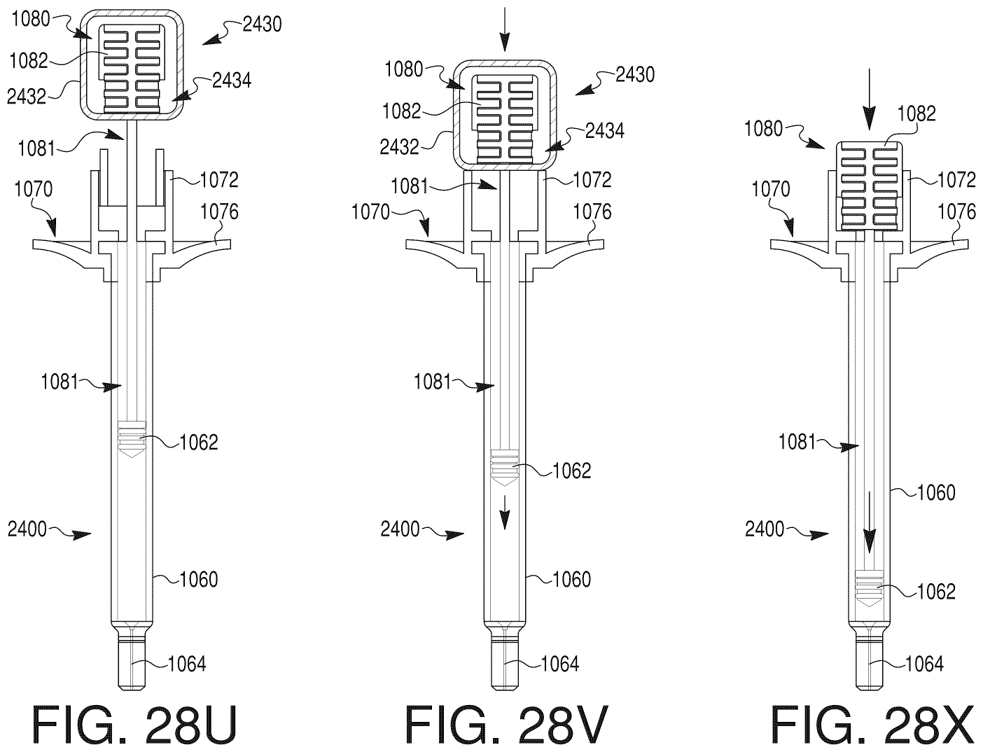

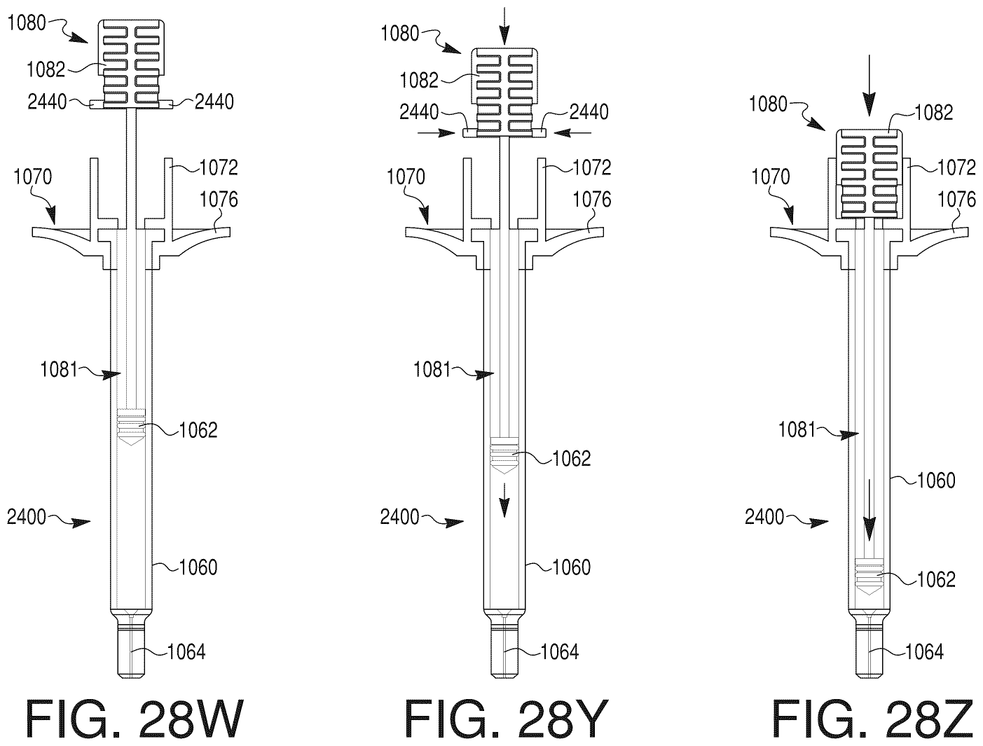

FIGS. 28A-28C depict an exemplary delivery device 2400 and method of using delivery device 2400. Delivery device 2400 may include substantially similar features as those shown and described above such that like reference numerals are used to identify like components. As shown in FIG. 28A, delivery device 2400 may include a removable clip 2402 coupled to body 1060 at a position distal to flange piece 1070. Removable clip 2402 may be an obstruction and/or blocking component configured to inhibit movement of flange piece 1070 relative to body 1060. Removable clip 2402 is selectively removable such that removable clip 2402 may be configured to disengage body 1060 in response to manual actuation of removable clip 2402.

By way of illustrative example, removable clip 2402 may have a body that wraps about an exterior of body 1060 and is configured to selectively deform (e.g., break, tear, etc.) upon application of a force thereto to decouple removable clip 2402 from body 1060. In other examples, removable clip 2402 may have a flexible body that is configured to bend in response to a radially-outward force being applied thereto, thereby disengaging removable clip 2402 from body 1060. By way of further example, removable clip 2402 may have a body that is configured to selectively transition between a closed configuration encapsulating a circumference of body 1060 therein and an open configuration permitting removal of body 1060 from the body of removable clip 2402. Removable clip 2402 may include various other suitable sizes, shapes, and/or configurations than those shown and described herein without departing from a scope of the present disclosure.

Delivery device 2400 may include a radial wall 1063 extending laterally outward from an exterior of body 1060, thereby forming an obstruction along body 1060. As seen in FIG. 28A, radial wall 1063 may be configured to inhibit distal translation of removable clip 2402 along body 1060. In some embodiments, radial wall 1063 may be an add-component attached to body 1060, while in other embodiments, radial wall 1063 may be integrally formed onto body 1060. Referring now to FIG. 28B, flange piece 1070 and plunger rod 1080 may be configured to translate distally along body 1060 to prime delivery device 2400 upon removal of removable clip 2402 from body 1060. In this instance, plunger rod 1080 may remain stationary relative to flange piece 1070, as the combined assembly of flange piece 1070 and plunger rod 1080 moves relative to body 1060. In other embodiments, plunger rod 1080 may remain stationary as flange piece 1070 translates distally along body 1060 to prime delivery device 2400. For example, at least a portion of flange piece 1070 may extend into body 1060 (e.g., and behind stopper 1062) when priming device 2400. In this instance, plunger rod 1080 may be translated separately to deliver a dosage from delivery device 2400.

With flange piece 1070 translated from a proximal position (FIG. 28A) to a distal position (FIG. 28B), delivery device 2400 may be in a primed position. It should be appreciated that body 1060 may be configured to limit movement by flange piece 1070 to a defined distance based on a location of radial wall 1063, which may correspond to a priming distance of delivery device 2400. Accordingly, a priming distance of delivery device 2400 may be controlled by adjusting a range of movement of flange piece 1070 along body 1060.

As seen in FIG. 28C, plunger rod 1080 may be translated distally relative to body 1060 in response to applying a distally-directed force onto actuation portion 1082. In this instance, stem 1081 may move relative to flange piece 1070, thereby causing stopper 1062 to move within body 1060 to deliver a dose. It should be appreciated that an extent that plunger rod 1080 translates relative to flange piece 1070 may define a dosage delivery distance of delivery device 2400. The dosage delivery distance may be controlled based on a gap formed between collar 1072 and actuation portion 1082.

In other embodiments, as seen in FIGS. 28D-28F, delivery device 2400 may further include a locking component, such as, for example, a removable rod 2404 coupled to flange piece 1070. Referring specifically to FIG. 28D, removable rod 2404 may be received through a proximal end of collar 1072, such as, for example, through one or more lateral apertures (not shown) formed through collar 1072. Removable rod 2404 may be configured to inhibit movement of plunger rod 1080 relative to flange piece 1070, such as, for example, preventing receipt of actuation portion 1082 into collar 1072. Removable rod 2404 may be selectively removable and configured to disengage collar 1072 upon manual actuation of removable rod 2404. It should be appreciated that delivery device 2400 may include various other locking components in addition to and/or in lieu of removable rod 2404, such as, for example, a pin, a tab, a bar, and the like.

For example, referring now to FIG. 28E, flange piece 1070 and plunger rod 1080 (e.g., stem 1081 and actuation portion 1082) may be configured to translate distally along body 1060 to prime delivery device 2400 in response to removal of removable clip 2402 from body 1060. Plunger rod 1080 may remain stationary relative to flange piece 1070 as the assembly of flange piece 1070 and plunger rod 1080 moves relative to body 1060. With flange piece 1070 translated from a proximal position (FIG. 28D) to a distal position (FIG. 28E), delivery device 2400 may be in a primed position. It should be appreciated that body 1060 may be configured to limit movement by flange piece 1070 to a defined distance based on a location of radial wall 1063 along body 1060, which may correspond to a priming distance of delivery device 2400.

As seen in FIG. 28F, removable rod 2404 may be disengaged from collar 1072 such that plunger rod 1080 is no longer inhibited from moving distally relative to flange piece 1070. Actuation portion 1082 may be translated into collar 1072 to move stem 1081 and stopper 1062 within body 1060 to deliver a dose. An extent that plunger rod 1080 translates relative to flange piece 1070 may define a dosage delivery distance of delivery device 2400.

In other embodiments, as seen in FIGS. 28G-28I, removable clip 2402 may be omitted entirely such that delivery device 2400 may include a single obstruction and/or blocking component, i.e., rod 2404. In this instance, flange piece 1070 may be fixed relative to body 1060. With actuation portion 1082 positioned proximally of rod 2404, delivery device 2400 may be primed in response to plunger rod 1080 translating distally toward flange piece 1070 until encountering rod 2404. It should be appreciated that flange piece 1070 and/or rod 2404 may be configured to inhibit distal translation of plunger rod 1080 relative thereto absent an application of a distally-directed force thereto. In other examples, delivery device 2400 may include a blocking component positioned between actuation portion 1082 and rod 2404 (e.g., removable clip 2402) to inhibit distal movement of plunger rod 1080.

Accordingly, a priming distance of delivery device 2400 may be defined by a distance between the distal end of actuation portion 1082 and rod 2404 when delivery device 2400 is in an assembled, pre-primed state (FIG. 28G). With actuation portion 1082 engaged against rod 2404, as seen in FIG. 28H, delivery device 2400 may be in a primed state. Rod 2404 may be removed from collar 1072 to thereby allow further translation of plunger rod 1080 distally relative to flange piece 1070. As shown in FIG. 28I, a dose may be delivered from delivery device 2400 in response to collar 1072 receiving actuation portion 1082. It should be appreciated that a longitudinal offset of a distal end of actuation portion 1082 and an inner surface of collar 1072 may be determinative to a dosage delivery distance. Accordingly, an extent (e.g., the dosage delivery distance) that plunger rod 1080 translates relative to flange piece 1070 may define a volume of dosage delivered by delivery device 2400.

In further embodiments, as shown in FIGS. 28J-28L, delivery device 2400 may include a fixed clip 2406 attached to body 1060 at a location relatively distal of removable clip 2402. Fixed clip 2406 may be an obstruction and/or blocking component positioned in contact with removable clip 2402 such that fixed clip 2406 may be configured to inhibit movement of removable clip 2402 along body 1060. With flange piece 1070 positioned proximally of removable clip 2402, fixed clip 2406 may be further configured to inhibit movement of flange piece 1070 when removable clip 2402 is positioned therebetween.

Referring now to FIG. 28K, flange piece 1070 may be configured to translate distally along body 1060 to prime delivery device 2400 upon removing removable clip 2402 from body 1060. In this instance, plunger rod 1080 may remain stationary relative to flange piece 1070 as the assembly of plunger rod 1080 and flange piece 1070 moves toward fixed clip 2406. With flange piece 1070 translated from a proximal position (FIG. 28J) to a distal position (FIG. 28K) engaged against fixed clip 2406, delivery device 2400 may be in a primed position. It should be appreciated that body 1060 may be configured to limit movement by flange piece 1070 to a defined distance, which may correspond to a priming distance of delivery device 2400.

As seen in FIG. 28L, plunger rod 1080 may be translated distally relative to body 1060 in response to applying a distally-directed force onto actuation portion 1082. Stem 1081 may move relative to flange piece 1070, causing stopper 1062 to move within body 1060 to deliver a dose. It should be appreciated that an extent that plunger rod 1080 translates relative to flange piece 1070 may define a dosage delivery distance of delivery device 2400. The dosage delivery distance may be controlled based on a position of fixed clip 2406 along body 1060.

In further embodiments, delivery device 2400 may include a sleeve 2408 extending distally from flange piece 1070, as shown in FIG. 28M. Sleeve 2408 may be attached to a distal end of flange piece 1070 and/or be integral with flange piece 1070, thereby forming a unitary structure. Sleeve 2408 may be disposed within body 1060 and include a distal end 2410. Sleeve 2408 may define a lumen that is sized and shaped to receive stem 1081 when plunger rod 1080 is coupled to flange piece 1070. As described in further detail herein, sleeve 2408 may be configured to move within a lumen of body 1060 in response to flange piece 1070 translating along an exterior of body 1060.

Sleeve 2408 may further include a locking component, such as, for example, a second protrusion 2412 formed along an interior surface of sleeve 2408 such that second protrusion 2412 extends at least partially into the lumen defined by sleeve 2408. In the embodiment, second protrusion 2412 is positioned relatively proximal of distal end 2410. In other embodiments, sleeve 2408 may include various other suitable locking components in lieu of second protrusion 2412, such as, for example, an opening sized, shaped, and configured to receive protrusion 1085.

Referring specifically to FIG. 28M, protrusion 1085 may extend radially outward from stem 1081 and positioned proximally relative to second protrusion 2412 when plunger rod 1080 is received through flange piece 1070 and sleeve 2408. To prime delivery device 2400, plunger rod 1080 may be translated distally relative to flange piece 1070 and sleeve 2408 until protrusion 1085 contacts second protrusion 2412. It should be appreciated that an extent that plunger rod 1080 translates relative to sleeve 2408 may define a priming distance of delivery device 2400. The priming distance may be controlled based on a position of protrusion 1085 and second protrusion 2412 relative to one another.

With protrusion 1085 engaged against second protrusion 2412 and a distal end of actuation portion 1082 received against an inner surface of collar 1072, plunger rod 1080 may be coupled to sleeve 2408 and delivery device 2400 may be in a primed state, as shown in FIG. 28N. Actuation portion 1082 may be fully received within collar 1072 and stem 1081 may be locked onto sleeve 2408. Accordingly, further translation of plunger rod 1080 may provide translation of flange piece 1070 and sleeve 2408 relative to body 1060. For example, as seen in FIG. 28O, plunger rod 1080 and flange piece 1070 may be translated distally relative to body 1060 in response to applying a distally-directed force onto actuation portion 1082. Stem 1081 may move relative to body 1060, causing stopper 1062 to move within body 1060 to deliver a dose.

Distal end 2410 may translate toward expulsion end 1064 as plunger rod 1080 and flange piece 1070 move distally until encountering fixed clip 2406. It should be appreciated that an extent that plunger rod 1080 and flange piece 1070 translate may define a dosage delivery distance of delivery device 2400. The dosage delivery distance may be controlled based on a position of fixed clip 2406 along body 1060.

In other embodiments, as seen in FIGS. 28P-28Q, delivery device 2400 may include an obstruction and/or blocking component in the form of a pull tab 2420. Pull tab 2420 may include a body 2422 having a circular-cross section defining a center opening 2424. Body 2422 may be formed of various flexible materials, including, for example, plastic, rubber, and the like. As described in further detail herein, pull tab 2420 may be frangible and/or deformable in response to an application of force onto body 2422. Pull tab 2420 may further include a graspable feature 2426 extending outwardly from body 2422 and configured to facilitate manual actuation of pull tab 2420. As seen in FIG. 28P, graspable feature 2426 may be integrally formed with body 2422 such that applying a radially-outward force (e.g., a pulling force) onto graspable feature 2426 may cause body 2422 to deform (e.g., tear, break, etc.), as shown in FIG. 28Q.

Referring now to FIG. 28R, pull tab 2420 may be secured to flange piece 1070 along a proximal end of collar 1072. Pull tab 2420 may be disposed over collar 1072 such that flange piece 1070 is separated from actuation portion 1082 by pull tab 2420 positioned therebetween. Stem 1081 may be received through center opening 2424 and into collar 1072 when body 2422 is attached to collar 1072. Pull tab 2420 may be configured to inhibit translation of actuation portion 1082 into collar 1072. A thickness and/or width of body 2422 may be sized such that a diameter of center opening 2424 is smaller than a diameter of actuation portion 1082 to block actuation portion 1082 from passing through pull tab 2420.

Delivery device 2400 may be primed in response to translating plunger rod 1080 distally relative to flange piece 1070 until encountering body 2422, as seen in FIG. 28S. It should be appreciated that an extent plunger rod 1080 translates relative to flange piece 1070 may correspond to a priming distance of delivery device 2400. The priming distance may be controlled based on a thickness of body 2422, thereby varying a relative distance between actuation portion 1082 and collar 1072. With actuation portion 1082 engaged against body 2422, graspable feature 2426 may be actuated to remove (e.g., break, tear, pull, etc.) pull tab 2420 from collar 1072. In this instance, body 2422 may be deformed (see FIG. 28Q) and disengaged from flange piece 1070, thereby permitting further translation of plunger rod 1080 distally relative to flange piece 1070.

As seen in FIG. 28T, actuation portion 1082 may be received within collar 1072 in response to applying a distally-directed force onto actuation portion 1082. Stem 1081 may move relative to body 1060, causing stopper 1062 to move within body 1060 to deliver a dose. It should be appreciated that an extent that plunger rod 1080 translates relative to collar 1072 may correspond to a dosage delivery distance of delivery device 2400. The dosage delivery distance may be controlled based on a thickness of pull tab 2420, thereby varying a relative distance between actuation portion 1082 and a distal (e.g., bottom) end of collar 1072.

In further embodiments, as shown in FIGS. 28U-28X, delivery device 2400 may include a removable cap 2430 coupled to plunger rod 1080. Removable cap 2430 may include a body 2432 defining a cavity 2434 that is sized and shaped to receive at least a portion of plunger rod 1080 therein (e.g., actuation portion 1082). Removable cap 2430 may include an opening along a bottom (e.g., distal) wall of body 2342 for receiving stem 1081. In some embodiments, removable cap 2430 may be attached to actuation portion 1082, while in other embodiments body 2342 may be directly coupled to stem 1081. Removable cap 2430 may be an obstruction and/or blocking component configured to increase a cross-sectional profile of actuation portion 1082 to inhibit movement of plunger rod 1080 relative to flange piece 1070, and more specifically to prevent translation of actuation portion 1082 into collar 1072.

Referring now to FIG. 28V, plunger rod 1080 may be configured to translate distally relative to flange piece 1070 to prime delivery device 2400 until a bottom wall of body 2432 encounters a proximal end of collar 1072. Removable cap 2430 may inhibit actuation portion 1082 from being received within collar 1072 due to at least a portion of body 2342 being disposed between actuation portion 1082 and collar 1072. With plunger rod 1080 translated from a proximal position (FIG. 28U) to a distal position (FIG. 28V) with body 2432 engaged against collar 1072, delivery device 2400 may be in a primed position. It should be appreciated that an extent that plunger rod 1080 translates relative to flange piece 1070 may correspond to a priming distance of delivery device 2400. The priming distance may be controlled based on a size of removable cap 2430 and/or a position of removable cap 2430 relative to plunger rod 1080. For example, in other embodiments, a bottom wall of body 2432 may be secured to a proximal portion of stem 1081 positioned relatively distal of actuation portion 1082. In this instance, a priming distance of delivery device 2400 may be reduced relative to that shown and described herein as body 2432 may be positioned in closer proximity to collar 1072. Accordingly, plunger rod 1080 may be required to move a smaller distance for removable cap 2430 to encounter collar 1072.

As seen in FIG. 28X, removable cap 2430 may be detached from plunger rod 1080 such that actuation portion 1082 may be exposed from body 2432. Plunger rod 1080 may be translated distally relative to body 1060 and received within collar 1072 in response to applying a distally-directed force onto actuation portion 1082. Stem 1081 may move relative to flange piece 1070, causing stopper 1062 to move within body 1060 to deliver a dose. It should be appreciated that an extent that plunger rod 1080 translates relative to flange piece 1070 may correspond to a dosage delivery distance of delivery device 2400. The dosage delivery distance may be controlled based on an attachment of removable cap 2430 relative to actuation portion 1082 and/or stem 1081 as described above. Further, a depth of collar 1072 may be determinative of the dosage delivery distance such that a size of collar 1072 may be adjusted accordingly to form various suitable dosage delivery distances.

For example, attaching removable cap 2430 such that a distal wall of removable cap 2430 is positioned flush against a distal end of actuation portion 1082 may increase a relative priming distance of delivery device 2400 by providing a longer separation between removable cap 2430 and collar 1072. Accordingly, the attachment position of removable cap 2430 may correspond to a smaller dosage delivery distance upon translating actuation portion 1082 into collar 1072 after removal of removable cap 2430. Alternatively, attaching removable cap 2430 such that the distal wall of removable cap 2430 is positioned distally from the distal end of actuation portion 1082 may decrease a relative priming distance, thereby providing a greater dosage delivery distance as actuation portion 1082 may require further longitudinal translation to be fully received within collar 1072. It should be appreciated that a size and/or shape of removable cap 2430 may vary to accommodate the various attachment positions described above.

In some embodiments, as shown in FIGS. 28W-28Z, delivery device 2400 may include one or more tabs 2440 secured to plunger rod 1080, such as, for example, along actuation portion 1082, stem 1081, and/or various other portions of plunger rod 1080. In the example, delivery device 2400 includes a pair of tabs 2440 extending radially outward from a distal end of actuation portion 1082. Tabs 2440 may be an obstruction and/or blocking component configured to increase a cross-sectional profile of actuation portion 1082 to inhibit movement of plunger rod 1080 relative to flange piece 1070, and more specifically to inhibit translation of actuation portion 1082 into collar 1072. In some embodiments, tabs 2440 may be selectively removable from actuation portion 1082 upon an application of force thereto. In other embodiments, tabs 2440 may be compressible and configured to be pushed into actuation portion 1082 in response to an application of force thereto. In either instance, tabs 2440 may be configured to transition actuation portion 1082 from an expanded profile (FIGS. 28W-28Y) to a compressed profile (FIG. 28Z).

Referring now to FIG. 28Y, plunger rod 1080 may be configured to translate distally relative to flange piece 1070 to prime delivery device 2400 until tabs 2440 encounter a proximal end of collar 1072. Tabs 2440 may inhibit collar 1072 receiving actuation portion 1082 therein. With plunger rod 1080 translated from a proximal position (FIG. 28W) to a distal position (FIG. 28Y) with tabs 2440 engaged against collar 1072, delivery device 2400 may be in a primed position. It should be appreciated that an extent that plunger rod 1080 translates relative to flange piece 1070 may correspond to a priming distance of delivery device 2400.

The priming distance may be controlled based on a size (e.g., thickness, width, height, etc.) of tabs 2440 and/or a position of tabs 2440 relative to plunger rod 1080. For example, in other embodiments, the pair of tabs 2440 may be secured to an intermediate and/or proximal portion of actuation portion 1082, or alternatively along stem 1081. In this instance, a priming distance of delivery device 2400 may be increased and/or decreased, respectively, relative to that shown and described herein.

As seen in FIG. 28Z, tabs 2440 may be compressed into actuation portion 1082 by collar 1072 applying an inward, pushing force thereto (or alternatively decoupled from actuation portion 1082 by applying an outward, pulling force, a rotating snapping force, or the like) such that actuation portion 1082 may form a smaller cross-sectional profile. Plunger rod 1080 may be translated distally relative to body 1060 and received within collar 1072 in response to applying a distally-directed force onto actuation portion 1082. Stem 1081 may move relative to flange piece 1070, causing stopper 1062 to move within body 1060 to deliver a dose. It should be appreciated that an extent that plunger rod 1080 translates relative to flange piece 1070 may correspond to a dosage delivery distance of delivery device 2400. As described above, the dosage delivery distance may be controlled based on a position of tabs 2440 relative to actuation portion 1082, a size (e.g., longitudinal depth) of collar 1072, and the like. For example, a relative position of tabs 2440 that increases a priming distance of delivery device 2400 may correspond to a smaller dosage delivery distance, and a position of tabs 2440 that corresponds to a reduced priming distance may provide a greater dosage delivery distance. In other examples, plunger rod 1080 may include a second set of tabs (not shown) along actuation portion 1082 which may define a dosage delivery distance based on a relative position of the tabs relative to tabs 2440.

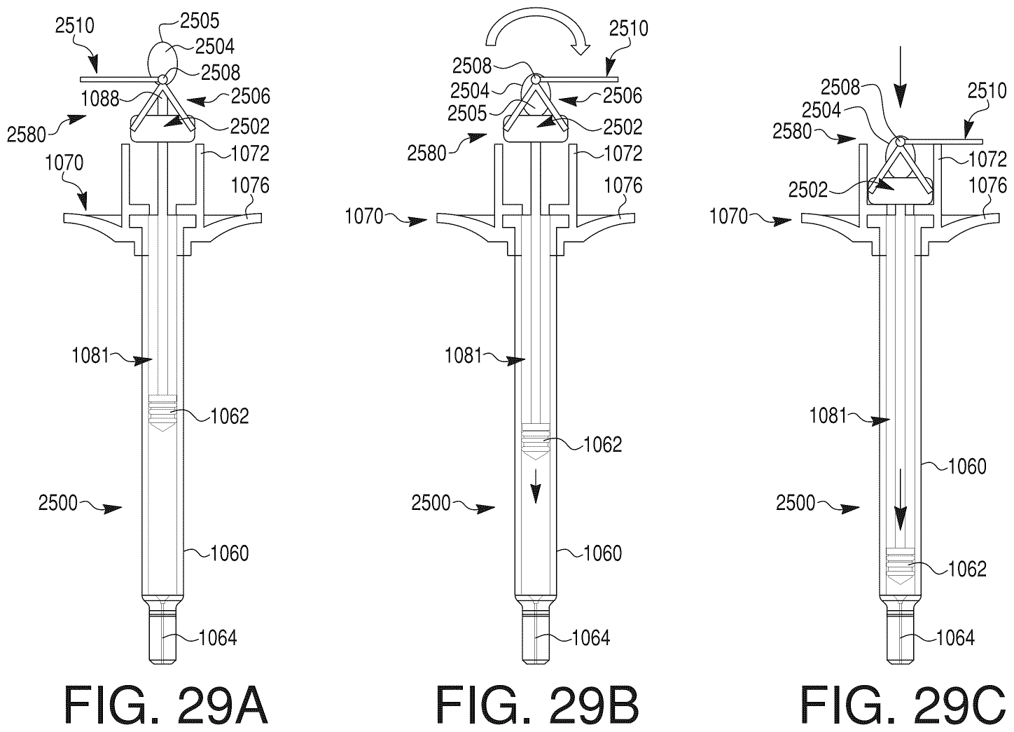

FIGS. 29A-29C depict an exemplary delivery device 2500 and method of using delivery device 2500. Delivery device 2500 may include substantially similar features as those shown and described above such that like reference numerals are used to identify like components. As shown in FIG. 29A, delivery device 2500 may include a plunger rod 2580 comprising a first actuation portion 2502, a second actuation portion 2504, and a cam lever 2510. First actuation portion 2502 may be coupled to second actuation portion 2504 by one or more arms 2506. In the example, a pair of arms 2506 may be fixed to first actuation portion 2502 along a first end of arms 2506, and arms 2506 may be further coupled to second actuation portion 2504 at a second end of arms 2506 that is opposite of the first end. Second actuation portion 2504 may be a rotatable element including a proximal end 2505 and an opposing distal end having a joint 2508. The pair of arms 2506 may be coupled to the distal end of second actuation portion 2504 at joint 2508.

It should be understood that, when in a ready position as seen in FIG. 29A, second actuation portion 2504 may be oriented such that joint 2508 is positioned proximate to first actuation portion 2502 relative to proximal end 2505. A proximal end 1088 of stem 1081 may be positioned adjacent to joint 2508 at a distal end of second actuation portion 2504. For example, proximal end 1088 may be in contact with and/or abut against the distal end of second actuation portion 2504. In some embodiments, stem 1081 may extend through a center of first actuation portion 2502 and/or be positioned alongside first actuation portion 2502. Second actuation portion 2504 may be configured to move relative to first actuation portion 2502 and about joint 2508. Cam lever 2510 may be coupled to second actuation portion 2504 at joint 2508 and configured to move (e.g., rotate, pivot, translate, etc.) second actuation portion 2504 relative to first actuation portion 2502. Accordingly, it should be appreciated that second actuation portion 2504 may be configured to move stem 1081 relative to body 1060 in response to cam lever 2510 moving second actuation portion 2504 relative to first actuation portion 2502.

For example, referring to FIG. 29A, cam lever 2510 may be actuated by rotating cam lever 2510 about joint 2508, thereby causing second actuation portion 2504 to rotate about joint 2508. Proximal end 2505 may be moved toward first actuation portion 2502 in response to second actuation portion 2504 rotating about joint 2508. In this instance, proximal end 2505 may be moved toward first actuation portion 2502. With proximal end 2505 moved from a proximal position (FIG. 29A) to a distal position (FIG. 29B), proximal end 1088 may be pushed distally, thereby translating stem 1081 relative to body 1060 to prime delivery device 2500. Stated differently, rotation of cam lever 2510 and/or second actuation portion 2504 relative to first actuation portion 2502 may prime delivery device 2500 by forcing stem 1081 distally.

It should be appreciated that a travel length of proximal end 2505 toward first actuation portion 2502 may correspond to a priming distance of delivery device 2500. In other words, a priming distance of delivery device 2500 may be controlled by a longitudinal length of second actuation portion 2504 between proximal end 2505 and joint 2508. In some embodiments, first actuation portion 2502, arms 2506, and/or cam lever 2510 may be to inhibit further rotation of second actuation portion 2504 after plunger rod 2580 is moved from the ready position (FIG. 29A) to the primed position (FIG. 29B).

As seen in FIG. 29C, plunger rod 2580 may be translated distally relative to body 1060 in response to applying a distally-directed force onto first actuation portion 2502 and second actuation portion 2504. In this instance, cam lever 2510 may be depressed (e.g., pushed and/or pulled) distally to move first actuation portion 2502 and second actuation portion 2504 toward flange piece 1070 until encountering a proximal end of collar 1072. Stem 1081 may move relative to collar 1072 thereby causing stopper 1062 to move within body 1060 to deliver a dose. It should be appreciated that an extent of translation of plunger rod 2580 relative to flange piece 1070 may correspond to a dosage delivery distance of delivery device 2500. The dosage delivery distance may be controlled based on a gap formed between collar 1072 and cam lever 2510.

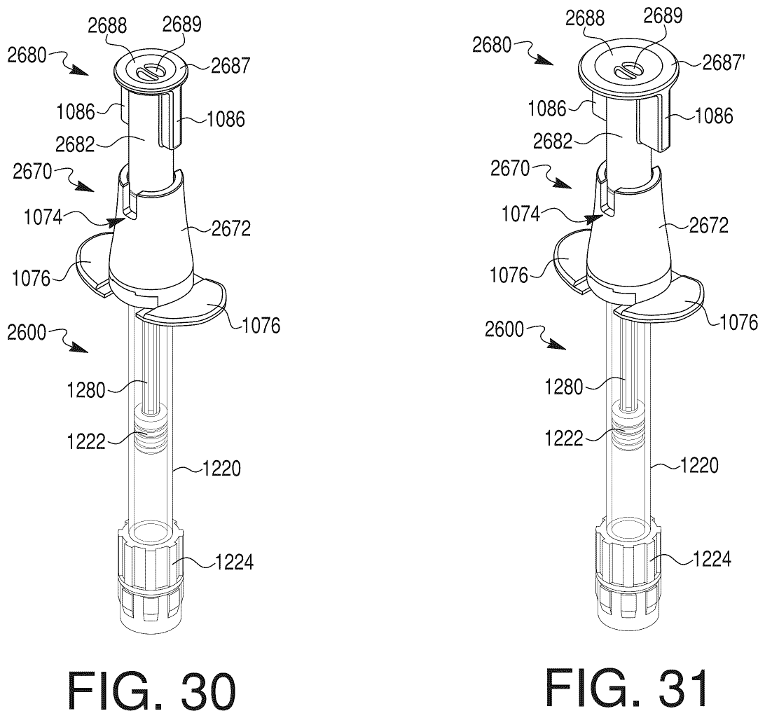

FIGS. 30-31 depict an exemplary delivery device 2600 that may include substantially similar features as those shown and described above such that like reference numerals are used to identify like components. Delivery device 2600 may include a flange piece 2670, a plunger rod 2680, and body 1220. Flange piece 2670 may include a tapered collar 2672 having a varying size and/or shape between a distal end and a proximal end. In the example, tapered collar 2672 may have a greater cross-sectional profile (e.g., diameter) along a distal end adjacent to flanges 1076 than at a proximal end adjacent to slots 1074. Tapered collar 2672 may be configured to minimize an overall profile and/or weight of delivery device 2600 by minimizing a configuration of flange piece 2670. In some embodiments, flanges 1076 may have a reduced length to facilitate enhanced control and maneuverability of flange piece 2670.

Plunger rod 2680 may include an actuation portion 2682 having a cross-sectional profile (e.g., diameter) that is relatively smaller than tapered collar 2672 to facilitate receipt of actuation portion 2682 therethrough. Accordingly, actuation portion 2682 may be similarly configured to minimize an overall profile and/or weight of delivery device 2600 by minimizing a configuration of actuation portion 2682. Further, plunger rod 2680 may omit inclusion of a textured and/or ribbed surface along actuation portion 2682 to simplify an exterior appearance of plunger rod 2680.

Referring specifically to FIG. 30, actuation portion 2682 may further include a proximal end having an outer ring 2687, an inner surface 2688, and one or more openings 2689 formed through inner surface 2688. In the example, inner surface 2688 may be disposed within outer ring 2687 and may have an angled profile that is sloped radially-inward toward the one or more openings 2689. Inner surface 2688 may be configured to define an interface for actuating plunger rod 2680 (e.g., applying a distally-directed force onto actuation portion 2682 by a finger of a user). Although plunger rod 2680 is shown as including a pair of openings 2689, it should be appreciated that in other embodiments additional and/or fewer openings 2689 may be included on inner surface 2688.

In some embodiments, as seen in FIG. 31, plunger rod 2680 may include an outer ring 2687′ having a width that defines an outer surface disposed about inner surface 2688. For example, an outer surface of outer ring 2687′ may be angled inwardly toward inner surface 2688 and openings 2689 and/or be transverse relative to inner surface 2688. In the present example, outer ring 2687′ defines a planar outer surface that is substantially perpendicular to a longitudinal length of actuation portion 2682. The enhanced width of outer ring 2687′ may provide additional surface area for a user of device 1050 to contact when actuating plunger rod 2680. It should be appreciated that a width of outer ring 2687′ may be greater and/or less than that shown and described herein without departing from a scope of this disclosure.

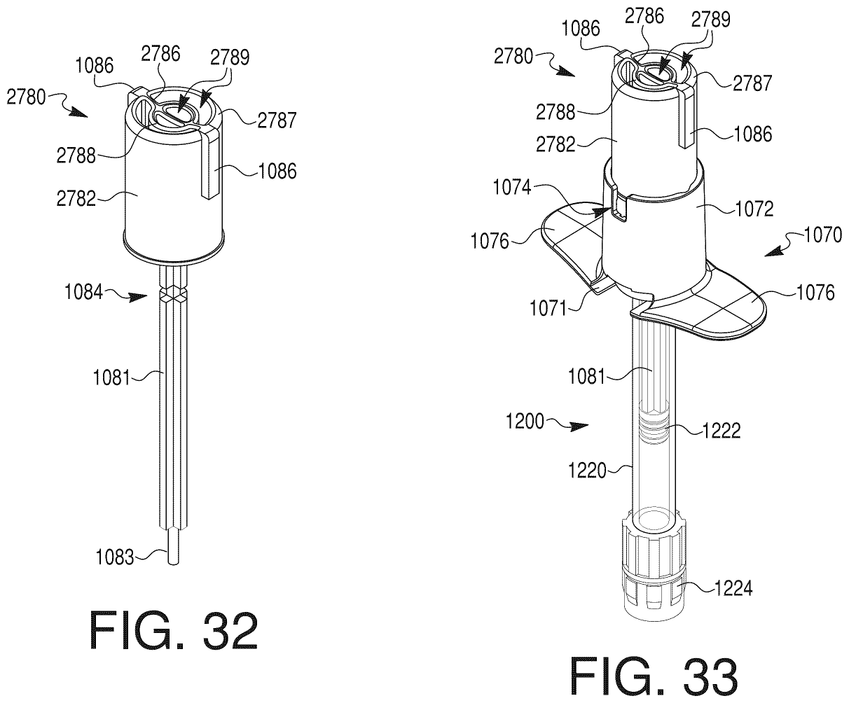

FIGS. 32-33 depict an exemplary plunger rod 2780 that may include substantially similar features as plunger rod 1080 shown and described above such that like reference numerals are used to identify like components. Plunger rod 2780 may include an actuation portion 2782 having a proximal end defined by an outer ring 2787, an inner ring 2788, and one or more openings 2789. In the example, inner ring 2788 may be disposed within outer ring 2787 and may define at least one opening 2789. Outer ring 2787 may further define at least one opening 2789 positioned radially outward of inner ring 2788. One or more of openings 2789 may minimize an overall weight of plunger rod 2780, enhance a molding manufacturing ability of plunger rod 2780 by providing nominal wall thicknesses for actuation portion 2782, and more. Additionally, actuation portion 2782 may include a lateral ledge 2786 extending across a width of the distal end and aligned with protrusions 1086. Lateral ledge 2786 may bifurcate the one or more openings 2789 defined by outer ring 2787 and inner ring 2788. Lateral ledge 2786 may be collinear with protrusions 1086 to provide visual alignment and/or identification of protrusions 1086 to a user of plunger rod 2780.

As seen in FIG. 33, with plunger rod 2780 received within flange piece 1070 and body 1220, lateral ledge 2786 may be configured to enhance an identification of movement by plunger rod 2780 relative to flange piece 1070 from a perspective proximal of device 1200. For example, lateral ledge 2786 may facilitate identifying a relative position of protrusions 1086 to slots 1074 from a perspective proximal to actuation portion 2782 during use of device 1200. In some embodiments, plunger rod 2780 may omit a textured and/or ribbed surface along actuation portion 2782 to simplify an exterior appearance of plunger rod 2780.

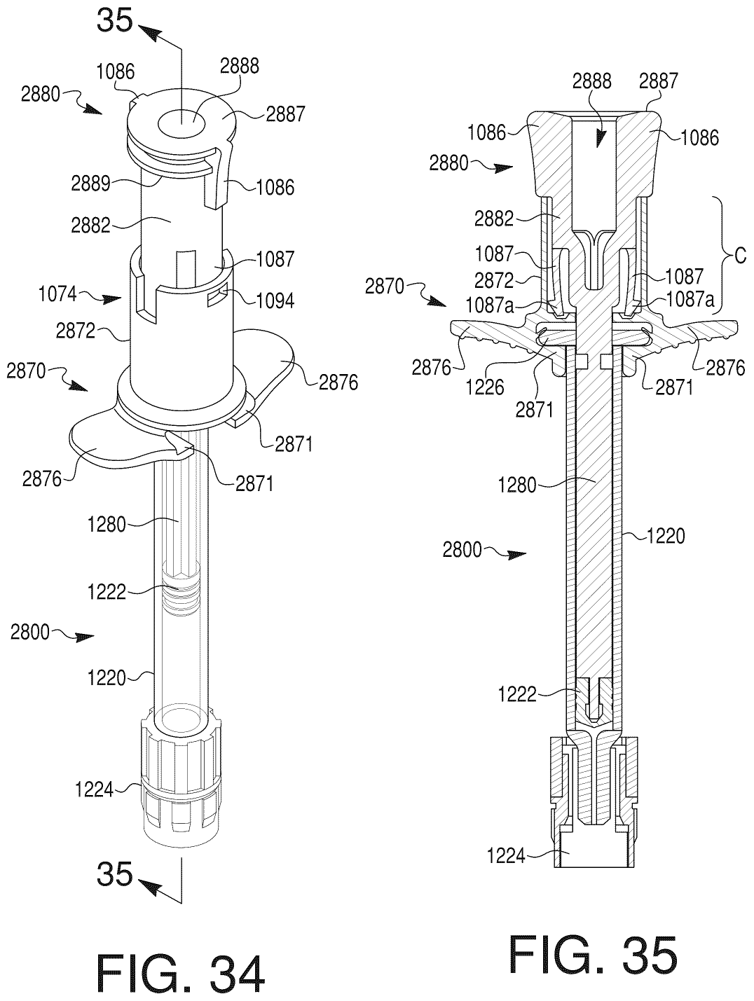

FIG. 34 depicts another exemplary delivery device 2800 in accordance with an example of this disclosure. Delivery device 2800 may include substantially similar features as delivery device 1050 and delivery device 1200 shown and described above such that like reference numerals are used to identify like components. Delivery device 2800 may include a flange piece 2870, a plunger rod 2880, stopper 1222, and body 1220. Body 1220 may include a nominal wall thickness of approximately 1.1 mm+/−0.1 mm. In some embodiments, a volume of body 1220 may be less than or equal to about 0.5 mL.

In some embodiments, stem 1280 of plunger rod 2880 may have a width of approximately 10.25 mm+/−0.08 mm. A longitudinal length extending from a proximal edge of protrusions 1086 on plunger rod 2880 to a distal edge of clip shaped parts (extensions) 1087a may be approximately 8.67 mm+/−0.1 mm. A longitudinal length extending from a distal end portion of protrusions 1086 to a distal end of stem 1280 may be approximately 56.79 mm+/−0.1 mm. A longitudinal length of plunger rod 2880 may be approximately 72.39 mm+/−0.2 mm. A longitudinal length extending from a distal edge of protrusion 1086 to a distal end portion of neck 1084 may be approximately 20.50 mm+/−0.1 mm.

In some embodiments, flange piece 2870 may have a width extending between the outer-lateral end of flanges 2876 of approximately 35 mm+/−0.2 mm. Collar 2872 of flange piece 2870 may have a depth of approximately 18.90 mm+/−0.1 mm. Slots 1074 of flange piece 2870 may have a depth of approximately 5.12 mm+/−0.1 mm. Collar 2872 of flange piece 2870 may have an outer diameter of approximately 13.11 mm+/−0.10 mm. Flange piece 2870 may have a longitudinal length extending of approximately 26.45 mm+/−0.10 mm.

In some embodiments, body 1220 may be coated with a siloxane, in which a general category of siloxanes may be used and may include material such as polydimethylsiloxane (PDMS). Siloxane may assist in binding silicone oil to the glass of body 1220, which also may be plasma treated upon application of the coating. For example, siloxane may be initially applied to the glass of body 1220 and then silicone oil may be applied prior to the barrel of body 1220 being plasma treated. Additionally and/or alternatively, stopper 1222 may be coated and/or non-coated with a material in lieu of and/or in addition to body 1220, such as but not limited to, silicone oil.

In some embodiments, body 1220 may include one or more coatings on the barrel. For example, a first coating of siloxane and a second coating of silicone oil may applied to an interior surface of body 1220, the second coating including an amount of approximately 0.2 mg+/−0.05 mg. In some embodiments, fewer or more coatings may be used on body 1220. In some embodiments, the glass of body 1220 may meet the requirements of USP <660> glass grain test. In further embodiments, the siloxane lubricant may include a viscosity at 25° C. of about 1000±5% mm2/s. Although body 1220 may be described as being formed of a glass material, it should be understood that body 1220 may be formed of various other suitable materials (e.g., polymers, plastic, etc.) without departing from a scope of this disclosure. In further embodiments, body 1220 may be made of a hybrid configuration in which one or more materials may be combined with one another, including but not limited to, a combination of glass and plastic.

In other embodiments, body 1220 of delivery device 2800 may be silicone-free, substantially silicone-free, or include baked-on silicone along an interior surface of body 1220. It should be appreciated that the term “substantially silicone-free” may correspond to a value of silicone-oil that is less than about 100 g, such as less than about 10 g of silicone-oil in the body 1220. Additionally, stopper 1222 may be silicone-free, substantially silicone-free, or include baked-on silicone along an exterior surface of stopper 1222. In some embodiments in which delivery device 2800 is silicone-free or substantially silicone-free, stopper 1222 may include polytetrafluoroethylene (PTFE).

In some embodiments, lubrication (e.g., silicone oil) may cause adverse effects on various medicament, alter immunologic response, and/or ultimately affect patient safety. A silicone-free and/or substantially silicone-free body 1220 and/or stopper 1222 may allow for a high degree of user-friendliness during medicament delivery from delivery device 2800. For example, body 1220 and/or stopper 1222 including a silicone-free or substantially silicone-free surface may reduce the risk of silicone-induced protein aggregation, particle formation, drug product precipitation, and/or may include a low break-loose and gliding force for moving stopper 1222 in body 1220. For example, between about 10-15% of large molecules may have decreased drug stability due to potential interaction with silicone. It should be appreciated that silicone migration may influence a consistency of delivery as break loose forces, glide forces, and injection times may vary with aging of the silicone oil. Additionally, silicone oil may cause aggregation and particle formation in therapeutic proteins, such as those described in the present disclosure.

Additionally, by being silicone-free and/or substantially silicone-free, stopper 1222 may be compliant with both glass or polymer (e.g., COC/COP) body 1220. In some embodiments, stopper 1222 may be formed of a medical grade thermoplastic elastomer to omit the inclusion of any lubrication and/or coating, such as silicone-oil, perfluoroalkyl and polyfluoroalkyl substances (PFAS), etc. In this instance, in lieu of stopper 1222 being formed of rubber which may generally include a coating with PFAS.

In addition, stopper 1222 may provide delivery device 2800 with increased closure integrity and a relatively lower break loose force after a long shelf life. For example, the shelf-life of delivery device 2800 including the medicament stored in body 1220 may be approximately 24 months, maintaining a stable condition when stored between approximately 2 to 8° C.

In the embodiments in which stopper 1222 may be silicone-free or substantially silicone-free, delivery device 2800 may be operable in protecting the complex and sensitive biologics from silicone-induced protein aggregation and particulation within body 1220, as briefly described above. In other words, stopper 1222 may omit any outer coating of silicone-oil and/or not involve silicone-oil in the manufacture of stopper 1222. Stopper 1222 may be configured to comply with USP <788> sub-visible particles standards, that may demonstrate potential orders of significant or magnitude reduction in sub-visible particles, excellent biocompatibility, and/or none or substantially no significant extractable compounds when a PTFE-based fluoropolymer barrier is applied to stopper 1222. Stopper 1222 may be configured to provide highly effective protection against drug loss, leakage, and microbial ingress. In further embodiments, stopper 1222 may include an ePTFE fluoropolymer barrier that is compatible with such therapeutic proteins, thereby preserving the particular concentration. By including a base glass (non-siliconized) body 1220 and silicone-free stopper 1222, drug delivery device 2800 may provide low sub-visible particles as a result. Embodiments of the present disclosure may be used in addition to and/or in combination with aspects of U.S. Pat. Nos. 8,722,178; 10,471,212; 11,020,531; 9,539,394; 10,493,207; and 11,439,757, each of which are incorporated by reference in their entirety herein.

In some embodiments, stopper 1222 may include a break-loose force of approximately 7.9 Newton (N) and a gliding force ranging between approximately 3 N to approximately 6 N. In addition, stopper 1222 may include a maximum injection force ranging between approximately 9 N to approximately 13 N. In some embodiments, stopper 1222 may be sized, shaped, and/or otherwise configured as a 0.5 mL piston, 1.0 mL piston, and/or other suitable sizes. In other embodiments, in addition to and/or in lieu of stopper 1222 being silicone-free or substantially silicone-free, stopper 1222 may be further configured to be tungsten-free and/or substantially tungsten-free. In some embodiments, stopper 1222 may include two or more ribs with a distance between the outermost ribs of about 2.5 mm.

It should be appreciated that glass prefilled syringes (e.g. body 1220) may be treated with silicone oil to lubricate the glass barrel in order to facilitate smooth movement of stopper 1222 during injection. In some instances, glass prefilled syringes (e.g. body 1220) may contain tungsten residues deposited during syringe manufacturing, specifically when forming the passage through which the drug is ejected from body 1220. Therefore, for example, after 8 mg aflibercept is filled into body 1220, the drug solution it may directly contact silicone oil and/or tungsten residues. The compatibility of aflibercept 8 mg formulation with silicone oil and/or tungsten residues was studied to confirm the compatibility of aflibercept with the PFS primary container configuration, and particularly body 1220.

As an illustrative example, the stability of aflibercept in the presence of silicone oil and tungsten was assessed using a DoE approach. The two factors included in the study were silicone oil concentration and tungsten concentration, and a 10-run study was designed which considered all main effects, interactions, and quadratics. In addition to the test formulations, one formulation was included as a control in which no silicone oil (i.e. silicone-free) or tungsten was added. As an exemplary finding, the addition of silicone oil up to 0.80 mg/mL and tungsten pin extracts up to 5.43 ppm had no meaningful impact on the physical or chemical stability of aflibercept 8 mg stored at 5° C. for up to 18 months. Under accelerated (25° C./60% RH) and thermal stress (37° C.) conditions, no new degradation products were observed, and the extent of degradation was not meaningfully affected by the presence of silicone oil up to 0.80 mg/mL and tungsten pin extracts up to 5.43 ppm. In the example, it is demonstrated that the presence of silicone oil and tungsten at up to 200% of the maximum concentrations found in the PFS primary container of body 1220 has no appreciable impact on the stability or potency of the aflibercept 8 mg drug product stored therein.

Flange piece 2870 may have a collar 2872 and a pair of flanges 2876 extending laterally outward from collar 2872. Collar 2872 may have a narrowed profile, such as, for example, relative to collar 1072. As illustrated, collar 2872 may be described and shown as including a generally round shape, however, additional shapes and/or sizes may be incorporated into collar 2872 without departing from a scope of this disclosure. For example, collar 2872 may be generally ovular in shape, including an elongated length relative to flanges 2876. In another example, collar 2872 may be generally triangular, including a relatively narrow profile. In some embodiments, flange piece 2870 may be made of polycarbonate and acrylonitrile butadiene styrene, or another suitable material within the scope of this disclosure.

In some embodiments, as described above, one or more of flange piece 2870 and/or plunger rod 2880 may be formed of various suitable materials, such as polycarbonate/acrylonitrile butadiene styrene, polypropylene homopolymer, an ABS (Acrylonitrile, Butadiene, and Styrene) polymer, ABS polycarbonate blend, and other suitable materials. Body 1220 may be formed of Type 1 borosilicate glass. Stopper 1222 may be formed of bromobutyl rubber. A tip cap of delivery device 2800 may be formed of polyisoprene.

It should be appreciated that such materials of said components of delivery device 2800 may provide biocompatibility such that they have been evaluated for one or more of cytotoxicity, sensitization, irritation or intracutaneous reactivity, material mediated pyrogenicity, acute system toxicity, and/or hemocompatability. In some embodiments, flange piece 2870, plunger rod 1280, body 1220, stopper 1222, and/or expulsion end 1224 may be manufactured in compliance with one or more applicable standards, such as ISO 10993-5, ISO 10993-10, and/or ISO 10993-23. In further embodiments, body 1220, stopper 1222, and/or expulsion end 1224 may be manufactured in compliance with ISO 10993-12, USP 151, ISO 10993-11, ISO 10993-4, and/or ASTM F756.

Additionally, flanges 2876 may have a shortened length relative to flanges 1076. Accordingly, flange piece 2870 of the present example may generally have a narrowed profile. Flange piece 2870 may further include a lip 2871 that may slide under or otherwise receive body flange 1226 (FIG. 35). Lip 2871 may be configured to hold flange piece 2870 in place by slidably coupling flange piece 2870 to body 1220. As described in further detail below, lip 2871 may be made of a flexible or semi-flexible material capable of forming a snap-fit connection with body flange 1226. As described in further detail below, flange piece 2870 may be configured to attach onto body 1220 prior to insertion of plunger rod 2880 into body 1220 during assembly of delivery device 2800.

Plunger rod 2880 may include an actuation portion 2882 having one or more protrusions 1086 along a proximal end and one or more extensions 1087 along a distal end. Actuation portion 2882 may have a diameter that is generally smaller than actuation portion 1082 shown and described above. In some embodiments, plunger rod 2880 may be made of polycarbonate and acrylonitrile butadiene styrene, or another suitable material within the scope of this disclosure. Accordingly, it should be appreciated that plunger rod 2880 and flange piece 2870 may collectively form a narrowed profile relative to an assembly of plunger rod 1080 and flange piece 1070. By providing a reduced profile, delivery device 2800 may be configured to provide a user enhanced control and maneuverability of plunger rod 2880 and flange piece 2870 during use of delivery device 2800.

In the embodiment, protrusions 1086 may have a curvature configured to enhance a grip, comfort, and/or ergonomics of plunger rod 2880 for a user of delivery device 2800. A curvature of protrusions 1086 may have a concave exterior configuration that taper inwardly along a distal portion of protrusions. A proximal end of actuation portion 2882 may further include a first ring 2887, an opening 2888, and a second ring 2889 positioned distally relative to first ring 2887. First ring 2887 may define a proximal interface of actuation portion 2882 and opening 2888 may be positioned at a center of first ring 2887. The proximal interface defined by first ring 2887 may be angled toward opening 2888 such that a proximal end of actuation portion 2882 may be sloped radially inward. In some embodiments, first ring 2887 may be sized, shaped, and configured to facilitate actuation of plunger rod 2880 by defining a finger pad for receiving a finger of a user. Opening 2888 may be configured to maintain a nominal wall thickness of actuation portion 2882 to facilitate molding of plunger rod 2880 during a manufacturing process of delivery device 2800. Openings 2888 may further minimize an overall weight of plunger rod 2880.

Still referring to FIG. 34, second ring 2889 may extend radially outward from an exterior surface of actuation portion 2882 and is positioned adjacent to first ring 2887. Second ring 2889 may be configured to form a graspable feature along actuation portion 2882 to enhance control of plunger rod 2880, such as, for example, when rotating plunger rod 2880. First ring 2887 may have a greater diameter than actuation portion 2882 such that the finger pad formed by first ring 2887 may have a greater cross-sectional profile than actuation portion 2882. In some embodiments, second ring 2889 may include a diameter greater than actuation portion 2882 and substantially similar to first ring 2887. Plunger rod 2880 may omit inclusion of a textured and/or ribbed surface along actuation portion 2882 to simplify an appearance of plunger rod 2880.

As seen in FIG. 35, actuation portion 2882 may be sized to have a predetermined length C between a distal end of protrusion 1086 and hook or clip shaped part 1087a of extensions 1087. In some embodiments, predetermined length C may be sized in accordance with a type and/or size of a syringe cap used with delivery device 2800 (e.g., Ompi Alba ITC, Ompi Alba OVS, Gerresheimer TELC, silicone-free syringes, etc.). For example, predetermined length C may be decreased and/or increased according to a lower and/or higher fill volume requirement, respectively, determined based on the syringe cap. Further, predetermined length C may be sized to provide a complete stroke of plunger rod 2880 into flange piece 2870 to ensure a complete dosage is delivered by delivery device 2800. The predetermined length C may be further adjusted to provide one of a plurality of suitable dosage delivery distances for delivery device 2800. Flange piece 2870 may include additional features and/or components configured to allow for a complete stroke of plunger rod 2880.

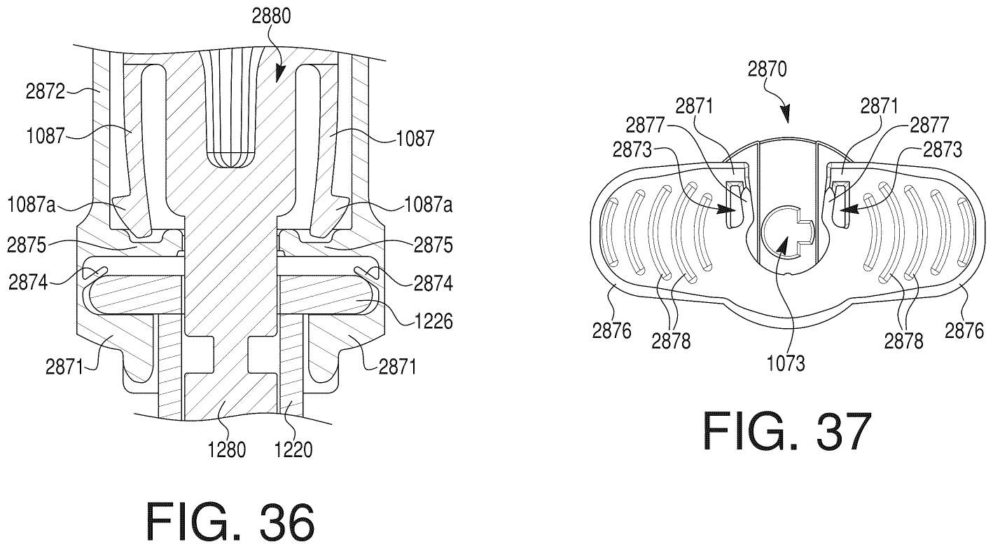

For example, referring now to FIG. 36, flange piece 2870 may include one or more indents 2875 formed along a proximally-facing and distally-located (bottom) surface of collar 2872. Indent 2875 may be sized and/or shaped to form a recessed surface into the bottom surface of collar 2872. Indent 2875 may be configured to facilitate receipt of plunger rod 2880 into flange piece 2870 to allow for a complete stroke. Stated differently, indent 2875 may provide an increased space and/or clearance within collar 2872 to receive one or more components of plunger rod 2880, such as, for example, hook or clip shaped part 1087a of extensions 1087.

In the present example, delivery device 2800 may be configured to deliver a complete dose (e.g., 0.5 mL, 1.0 mL, etc.) upon the pair of protrusions 1086 contacting a distal end (the bottom) of slots 1074. The pair of extensions 1087 may be positioned adjacent to (but not in contact with) a bottom surface of collar 2872 when protrusions 1086 contact the distal end of slots 1074. That is, in some embodiments, extensions 1087 may positioned proximal to the bottom surface of collar 2872 such that extensions 1087 do not contact the bottom surface when plunger rod 2880 has bottomed out and/or when a complete dose has been delivered from delivery device 2800. By forming a depression along the bottom surface of collar 2872, indent 2875 may allow actuation portion 2882 to translate distally relative to collar 2872 to complete a full stroke of plunger rod 2880 without extensions 1087 engaging or contacting the bottom surface of collar 2872. In some embodiments, extensions 1087 may bend inwardly toward indent 2875 upon hook or clip shaped parts 1087a encountering the bottom surface of collar 2872, thereby guiding hook or clip shaped parts 1087a into indent 2875. It should be appreciated that an increased space formed by indent 2875 may ensure extensions 1087 are not prevented from contacting the bottom surface of collar 2872 to complete the full stroke of plunger rod 2880 and/or to deliver a complete dose.

Still referring to FIG. 36, flange piece 2870 may further include one or more ribs 2874 configured to engage body flange 1226 when body 1220 is coupled to flange piece 2870. The one or more ribs 2874 may be positioned adjacent to lip 2871, such as, for example, distally of the bottom surface of collar 2872 and proximally of lip 2871. In some embodiments, ribs 2874 may extend radially inward from an inner sidewall of flange piece 2870, while in other embodiments ribs 2874 may extend outwardly from an inner top wall of flange piece 2870. In the present example, ribs 2874 may extend radially inward at an angle relative to the inner sidewall of flange piece 2870. It should be appreciated that ribs 2874 may be positioned and/or extend from various other suitable locations, and at various other suitable angles, within flange piece 2870 for engaging body flange 1226.

In the embodiment, ribs 2874 may be formed of a flexible and/or semi-flexible material (e.g., plastic, rubber, etc.) and configured to interact with body flange 1226 upon receipt of body 1220 within flange piece 2870. By way of illustrative example, ribs 2874 may be configured to flex and/or bend proximally toward a bottom surface of collar 2872 in response to lip 2871 receiving body flange 1226. Ribs 2874 may be operable to secure body flange 1226 to flange piece 2870 by applying a distally-directed force thereto. Accordingly, ribs 2874 may secure a position (e.g., longitudinal, rotational, etc.) of body 1220 relative to flange piece 2870 by engaging a top/proximal surface of body flange 1226 as lip 2871 engages a bottom surface of body flange 1226. In other embodiments, additional and/or fewer ribs 2874 may be included for inhibiting movement of body flange 1226 and/or body 1220 relative to flange piece 2870.

Referring now to FIG. 37, flange piece 2870 may include a textured and/or patterned interface 2878 along a bottom, distally-facing surface of flanges 2876. Textured interface 2878 may include one or more protrusions, depressions, and/or various other features forming at least one of a plurality of patterns to enhance a grip, control, and/or ergonomics of flange piece 2870. In the example, textured interface 2878 includes a plurality of semi-circular protrusions of varying sizes. As shown in FIG. 37, each interface 2878 may be concave when viewed from a radial center of flange 2876. However, in alternate embodiments, one or more interface 2878 may be convex when viewed from the radial center of flange 2876. As described in further detail below, textured interface 2878 may include various other designs, features, and/or patterns along the bottom surface (see FIGS. 41A-41D) of flanges 2876. Flange piece 2870 may further include a pair of movable tabs 2877 positioned adjacent to lip 2871 and along opposing sides of opening 1073. Movable tabs 2877 may be formed of a flexible and/or semi-flexible material and may be configured to move relative to collar 2872 and/or flanges 2876 in response to a force being applied thereto (e.g., by body 1220).

Each movable tab 2877 may define an opening 2873 disposed between movable tab 2877 and flange 2876. Accordingly, movable tabs 2877 may be separated from flanges 2876 by opening 2873 formed therebetween. Openings 2873 may provide a gap and/or clearance space to accommodate lateral movement of movable tabs 2877 upon receiving a radially-outward directed force. For example, movable tabs 2877 may be deflected radially outward toward flanges 2876 in response to flange piece 2870 receiving body 1220 through opening 1073, thereby changing a size and/or shape of openings 2873. In this instance, movable tabs 2877 may bend outwardly away from opening 1073 until body flange 1226 is received by lip 2871. Movable tabs 2877 may be configured to bend inwardly toward body 1220 to return to an original configuration upon lip 2871 fully receiving body flange 1226 therein. In some embodiments, movable tabs 2877 may bend toward body 1220 to a substantially originally configuration such that movable tabs 2877 may remain at least partially compressed against body 1220 to inhibit movement of body 1220 relative to flange piece 2870 to allow pressure to be continually applied onto body 1220 to prevent slippage.

Still referring to FIG. 37, movable tabs 2877 may be configured to apply a radially-inward directed force onto body 1220 (e.g., with a radially-inward directed material bias), thereby forming a snap-fit connection between flange piece 2870 and body 1220. Additionally, movable tabs 2877 may maintain body 1220 in a stabilized and fixed position relative to flange piece 2870, thereby coupling flange piece 2870 to body 1220. It should be appreciated that openings 2873 may be included between movable tabs 2877 and flanges 2876 to decrease a required force to couple body 1220 to flange piece 2870. For example, openings 2873 may be operable to reduce a force necessary to snap body 1220 into flange piece 2870 by a minimum force ranging from about 15 Newton to about 25 Newton, compared to a design omitting openings 2873.

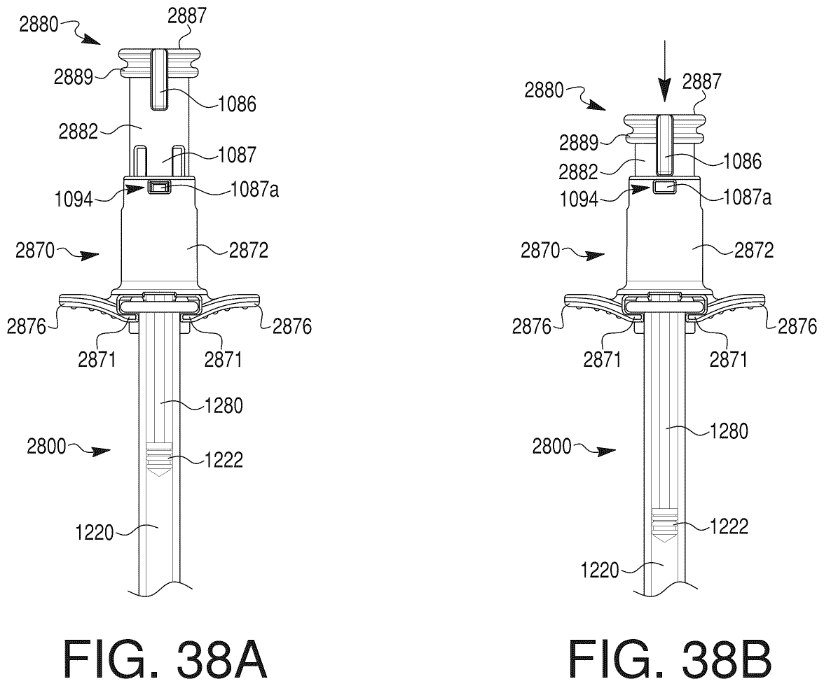

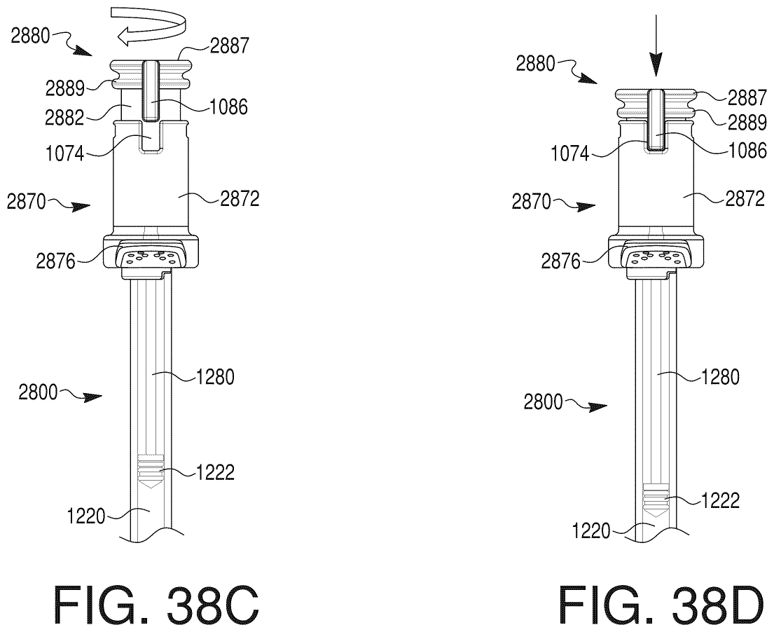

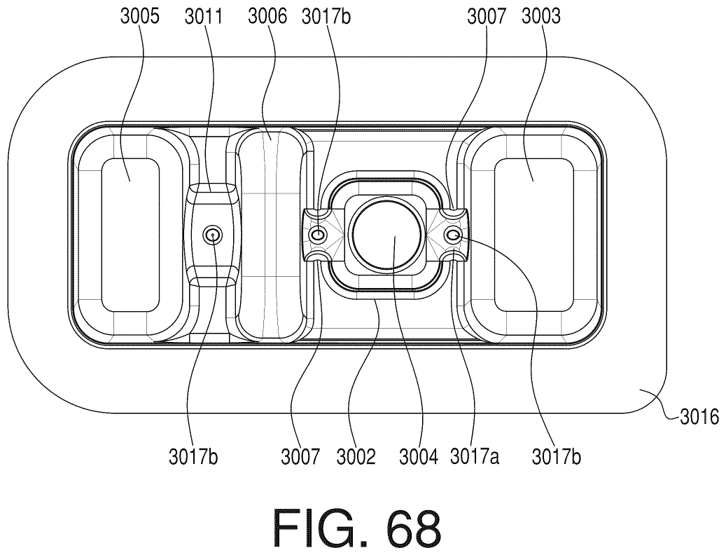

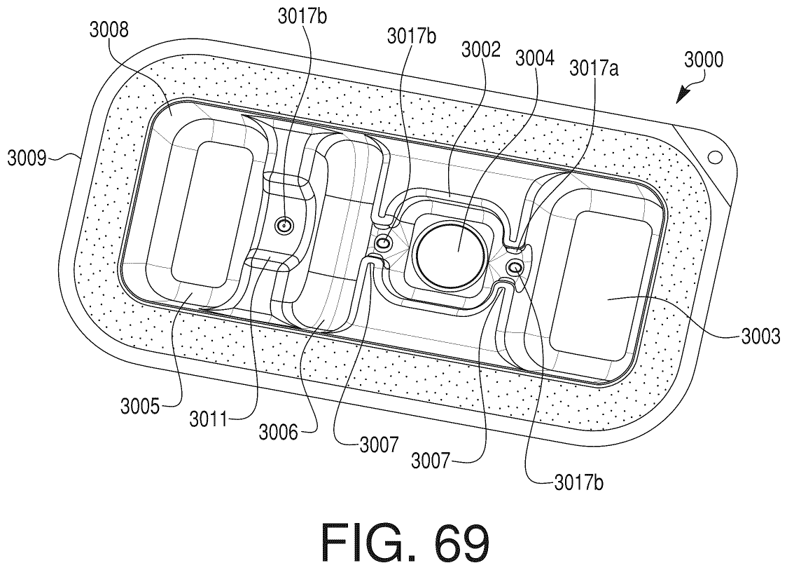

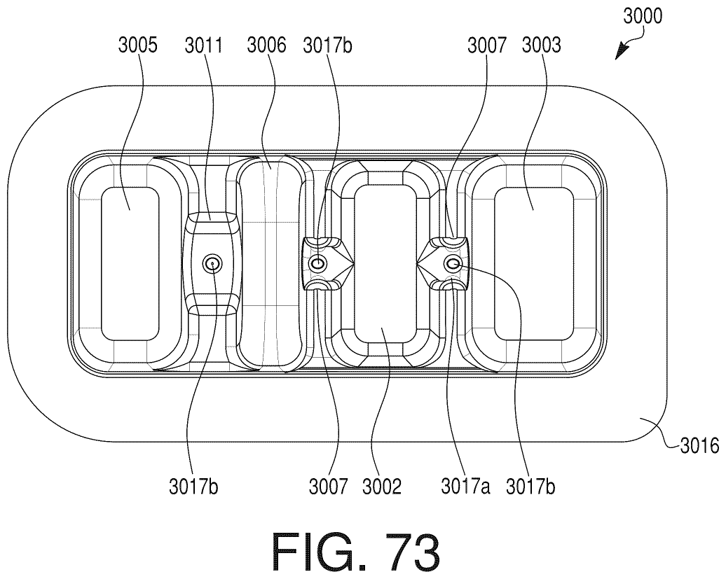

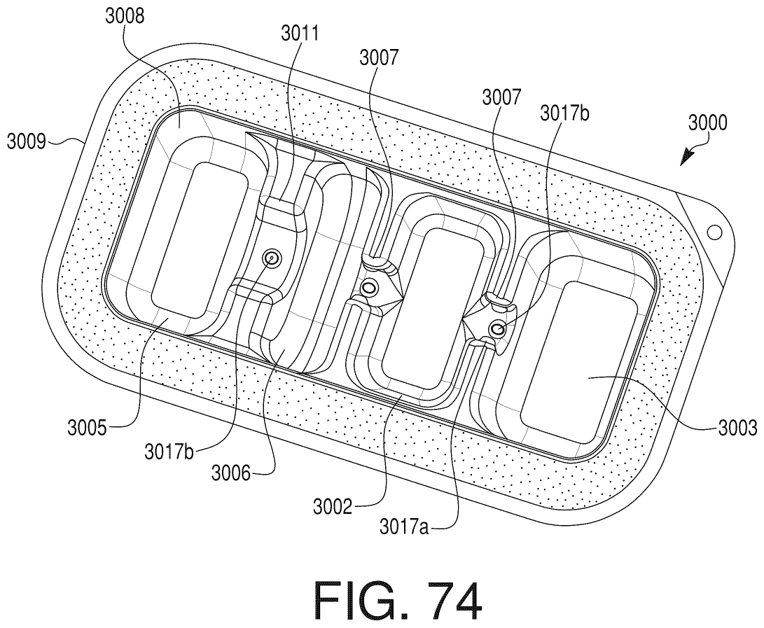

FIGS. 38A-40C show an illustrative method of using delivery device 2800. Prior to use delivery device 2800, a user may visually check the description of the packaging to ensure the correct dose is contained within body 1220 and the expiration date has not elapsed. The user may continue to open a packaging (e.g., packaging 3000) housing delivery device 2800 therein, as described in further detail below in reference to FIGS. 58-77B, by peeling open a removable cover (e.g., a Tyvek® lid) that ensure the sterility of its contents. Upon removing delivery device 2800 from its packaging, the user may further inspect delivery device 2800 and the medicament stored therein. The user may assemble at least one portion of delivery device 2800, including snapping off the syringe cap from body 1220 and attaching an injection needle (e.g., staked needle, luer needle, etc.) onto an expulsion end 1224 (e.g., a syringe tip) of body 1220. As described further herein, delivery device 2800 may be configured and operable to couple with a staked needle, a luer needle, and various other suitable needles at expulsion end 1224 without departing from a scope of this disclosure.

In addition, the user may check and/or inspect body 1220 for preliminary removal of any air bubbles that may have formed during assembly of delivery device 2800, such as by tapping body 1220 with a finger of the user, prior to priming delivery device 2800. As described in detail below, flange piece 2870 may be configured to attach onto body 1220 prior to an insertion of plunger rod 2880 into body 1220 during assembly of delivery device 2800. As seen in FIG. 38A, delivery device 2800 may be preassembled with a distal portion of actuation portion 2882 received within collar 2872 and extensions 1087 received within and coupled to openings 1094. With extensions 1087 coupled to collar 2872 via openings 1094, it should be appreciated that flange piece 2870 may inhibit proximal retraction of actuation portion 2882. Accordingly, disassembly of plunger rod 2880 from flange piece 2870 may be prevented. In this instance, delivery device 2800 may be primed by distally translating plunger rod 2880 into flange piece 2870 to prime delivery device 2800, as described in further detail below with reference to FIG. 38B.

In some embodiments, delivery device 2800 may include one or more markings and/or indicia to indicate whether delivery device 2800 is primed and/or ready for use. For example, upon insertion of plunger rod 2880 into body 1220, plunger rod 2880 may include one or more markings or indicia at one or more locations to provide a visual indication to a user of delivery device 2800 as to a current status and/or operating condition of delivery device 2800. One or more markings may include numbers, lines, colors, or various other visual depictions to represent the current status and/or positional location of the plunger rod 2880 with respect to delivering medicament from delivery device 2800. Although plunger rod 2880 is described herein as including one or more markings or indicia, it should be appreciated that additional and/or other components of delivery device 2800 (e.g., body 1220, stopper 1222, actuation portion 2882, and/or flange piece 2870) may include the one or more markings or indicia to assist in the determination of the status of delivery device 2800.

In some embodiments, stopper 1222 may be configured to be inserted into body 1220 and may be configured to hold a predetermined volume of a formulated drug substance inside body 1220, such as between stopper 1222 and expulsion end 1224 of body 1220. Expulsion end 1224 may be coupled to body 1220 at a distal end of body 1220. Expulsion end 1224 as depicted may include a snap of tip. For example, a snap tip may be removed from body 1220 by way of snapping, breaking, fracturing, or any other suitable method of removal. In other embodiments, expulsion end 1224 may include a luer tip in place of a snap tip. For example, a luer tip may be removed from body 1220 by way of rotating, twisting, turning, spinning, or any other suitable method of removal.

In some embodiment, stopper 1222 may be configured to utilize a particular color (e.g., blue) in order to present a visual indication between the other components of delivery device 2800 (e.g., body 1220, flange piece 2870, etc.) and stopper 1222 and/or with the environmental surroundings (e.g., sterilization trays, medicament, operating table or room, etc.) of delivery device 2800. For example, stopper 1222 may be configured to include a generally blue color to distinguishably differentiate it with respect to the other components of delivery device 2800 during assembly and use. In addition, the color may be configured to visually represent and/or verify the location of stopper 1222 to a user when movably inserted into body 1220, providing a visual identification of a current level at which the medicament contained therein is located based on the position of stopper 1222 in body 1220. It should be understood that a position of stopper 1222 within body 1220 may be indicative of a volume and/or amount of medicament remaining in body 1220 as stopper 1222 may be sized, shaped, and/or otherwise configured to be positioned adjacent to, against, and/or in contact with an upper surface of the medicament stored in body 1220.

In some embodiments, stopper 1222 may be the same or a substantially similar color to one or more other components (e.g., flange piece 2870) of delivery device 2800 to facilitate use of delivery device 2800. For example, stopper 1222 and flange piece 2870 may be configured using a vibrant blue color. In this example, the medicament stored in body 1220 may be configured as a clear and/or translucent structure such that incorporating stopper 1222 and/or flange piece 2870 in a vibrant blue color may assist the user in readily determining the level of medicament within body 1220 prior to, during, and/or after using delivery device 2800. Although the color blue is described herein, it should be appreciated that other additional colors, textures, and/or visual effects (e.g., reflective materials and/or coatings) may be employed in delivery device 2800 to facilitate and present a visual indication of stopper 1222 with respect to its surroundings and other components described herein, including body 1220.

In some embodiments, expulsion end 1224 may include a stake needle (not shown). Stake needles may be configured to be glued or adhered to the end of body 1220 to be held in place at expulsion end 1224. In some embodiments, stake needles may be initially installed prior to the addition of medicament into the body of the syringe as discussed in further details below. In silicone based syringes, the adhesion of the stake needle to the syringe body may harmed due to the baking process, thereby rendering delivery device 2800 inoperable for use. However, in silicone-free or substantially silicone-free delivery device 2800 that does not utilize a baking process, staked needles may be utilized with body 1220. Providing staked needles on delivery device 2800 may involve less steps during assembly for a user when preparing delivery device 2800 for use, provide less dead and/or empty space between the medicament stored in body 220 and the tip of the staked needle. In this instance, body 1220 may not require additional medicament to be prefilled (e.g., overfill) therein. Staked needles may further provide improved handling, reduced risk of contamination, enhanced user convenience, and/or reduced administration time when using delivery device 2800.

In some embodiments, the use of a cap surrounding the staked needle tip may be employed. The cap may be configured to assist the user with insertion of the staked needle into body 1220 during assembly. The cap may be configured to limit, restrict, and/or otherwise prevent contact between the tip of the staked needle and the sealing material of the cap (e.g., rubber). In this instance, the tip of the staked needle may not interact with or become deformed during assembly onto body 1220. The cap may reduce potential contamination of body 1220 in the tip region of the staked needle. In some embodiments, during injection the staked needle may at least partially retract through stopper 1222 and into body 1220, whereas in other embodiments the staked needle may be fixedly secured.

In other embodiments, expulsion end 1224 may include a luer needle (not shown) assembled thereto. The luer needle may be employed with a luer cone and/or a luer lock syringe systems, each one having various configurations of the proximal flange for connection with body 1220. Luer needles may include needle of choice flexibility. Additionally, the size, shape, and/or configuration of either a staked needle or a luer needle may be modified based on the need of the use application for delivery device 2800. For example, a shorter length for the needle may provide enhanced strength, thereby rendering the needle less prone to bending. The needle may include relatively thin walls providing lower injection force of the needle into the subject during use of delivery device 2800. Additionally and/or alternatively, low dead space may be provided to allow for relatively less overfilling of medicament in body 1220, as described above.

As seen in FIG. 38B, actuation portion 2882 may be translated distally relative to flange piece 2870 until protrusions 1086 encounter a proximal end of collar 2872. Stated differently, plunger rod 2880 may be translated by an initial distal translation (i.e. a first push) relative to flange piece 2870 and body 1220. For example, when actuation portion 2882 is translated distally relative to flange piece 2870, air and/or excess volume of medicament within body 1220 may be expelled from delivery device 2800. Plunger rod 2880 may complete a priming process of delivery device 2800 upon protrusions engaging and/or abutting collar 2872. Stated differently, to prime delivery device 2800, a user may push on plunger rod 2880 until protrusions 1086 reach or make contact with a proximal end side of collar 2872. It should be appreciated that an extent that plunger rod 2880 translates distally relative to flange piece 2870 may correspond to a priming distance of delivery device 2800. The priming distance may be controlled based on a longitudinal length of protrusions 1086 and/or extensions 1087, thereby varying a relative distance between the proximal end of collar 2872 and a distal end of protrusions 1086. As described above, one or more markings or indicia may be present on one or more components (e.g., body 1220, stopper 1222, actuation portion 2882, and/or flange piece 2870) of delivery device 2800 to assist in determining the status of delivery device 2800. For example, upon plunger rod 2880 translating in a distal direction with respect to flange piece 2870 and body 1220 via the first push, delivery device 2800 may be in a primed status. The one or more markings or indicia may be configured to visually indicate to a user the primed status of delivery device 2800.

As shown in FIG. 38C, flange piece 2870 may be rotated relative to plunger rod 2880, or vice versa, to move protrusions 1086 relative to collar 2872 until arriving into radial and longitudinal alignment with slots 1074. Stated differently, plunger rod 2880 may be rotated (e.g., twisted) relative to collar 2872 to move protrusions 1086 in alignment with slots 1074 after completion of the first (distal) push described above. In addition, the user may set a dose of medicament by turning plunger rod 2880, such as approximately 90 degrees clockwise or counterclockwise relative to flange piece 2870 and/or body 1220, until protrusions 1086 align with slots 1074. In this instance, delivery device 2800 may be configured to generate a tactile and/or audible feedback, such as a physical vibration and/or a “click” sound, to indicate such alignment of components to the user of delivery device 2800. In some embodiments, delivery device 2800 may include a maximum use torque defined as the maximum torque required to rotate plunger rod 2880 into a primed position. The maximum use torque may include an intra-day precision maximum torque of approximately 2 N·cm with a standard deviation of approximately 0.2 N·cm, and an intermediate precision maximum torque of approximately 3 N·cm with a standard deviation of approximately 0.2 N·cm. In some embodiments, delivery device 2800 may include a maximum use torque of 13 N·m.

An instrument may be used to measure the maximum use torque required to rotate the plunger rod into the “Ready to Dose” state after priming. Maximum use torque is measured at prefilled syringe drug product release and end-of-shelf-life to ensure that the maximum torque required to rotate the plunger rod does not exceed the capability of the 5th percentile female to remove a twist off cap with a dry hand. The dye leak test measures the integrity of the blister package seal and is monitored in accordance with USP <1207.2> Packaging Integrity Leak Test Technologies and ASTM Chapter F1929—15 “Standard Test Method for Detecting Seal Leaks in Porous Medical Packaging by Dye Penetration.” The testing is performed using a dye ingress method and the result is expressed as either “pass” or “fail.” The acceptance criteria are set as “no dye detected.”

As described above, one or more markings or indicia may be present on one or more components (e.g., body 1220, stopper 1222, actuation portion 2882, and/or flange piece 2870) of delivery device 2800 to assist in determining the status of delivery device 2800. For example, upon plunger rod 2880 being rotated with respect to collar 2872 for moving protrusions 1086 into alignment with slots 1074, delivery device 2800 may be in a delivery ready status. The one or more markings or indicia may be configured to indicate to a user the delivery ready status of delivery device 2800.

As shown in FIG. 38D, actuation portion 2882 may be translated distally relative to flange piece 2870 until protrusions 1086 encounter a proximal end of slots 1074. Stated differently, plunger rod 2880 may be translated by a second distal translation (i.e. a second push) relative to flange piece 2870 and body 1220 for delivering medicament out of the expulsion end 1224. During use, delivery device 2800 may include a break force, a glide force (e.g., sustaining force), and a maximum force (e.g., maximum injection force).

Break force may be defined as the amount of force required to overcome static friction and any adhesion between plunger rod 2880 and a glass wall of body 1220. An exemplary study was performed using approximately 36 syringes to determine an intra-day precision break force for VEGF high concentration DP PFS over approximately three days. Based on the study, for day 1, the minimum break force was approximately 3.7 N, the maximum break force was approximately 5.3 N, and the average break force was approximately 4.4 N with a standard deviation of approximately 0.3 N. For day 2 of the study, the minimum break force was approximately 3.8 N, the maximum break force was approximately 5.1 N, and the average break force was approximately 4.4 N with a standard deviation of approximately 0.3 N. For day 3 of the study, the minimum break force was approximately 3.9 N, the maximum break force was approximately 5.1 N, and the average break force was approximately 4.5 N with a standard deviation of approximately 0.3 N. The overall average break force from the multi-day study was approximately 4.4 N with an overall standard deviation of approximately 0.30 N.

Glide force may be defined as the amount of force required to maintain movement of stopper 1222 at the speed defined in the method (e.g., force to overcome dynamic friction). An exemplary study was performed using approximately 36 syringes to determine an intra-day precision glide force for VEGF high concentration DP PFS over approximately three days. Based on the study, for day 1, the minimum glide force was approximately 5.9 N, the maximum glide force was approximately 8.6 N, and the average glide force was approximately 7.7 N with a standard deviation of approximately 0.6 N. For day 2 of the study, the minimum glide force was approximately 6.3 N, the maximum glide force was approximately 8.6 N, and the average glide force was approximately 7.6 N with a standard deviation of approximately 0.5 N. For day 3 of the study, the minimum glide force was approximately 6.3 N, the maximum glide force was approximately 8.8 N, and the average glide force was approximately 7.5 N with a standard deviation of approximately 0.6 N. The overall average glide force from the multi-day study was approximately 7.6 N with an overall standard deviation of approximately 0.6 N.

Maximum force may be defined as the maximum force obtained at any point during the injection stroke of the second (distal) push. An exemplary study was performed using approximately 36 syringes to determine an intra-day precision maximum force for VEGF high concentration DP PFS over approximately three days. Based on the study, for day 1, the minimum (maximum) force was approximately 10.4 N, the maximum (maximum) force was approximately 12.2 N, and the average (maximum) force was approximately 11.2 N with a standard deviation of approximately 0.30 N. For day 2 of the study, the minimum (maximum) force was approximately 9.8 N, the maximum (maximum) force was approximately 12.1 N, and the average (maximum) force was approximately 10.7 N with a standard deviation of approximately 0.4 N. For day 3 of the study, the minimum (maximum) force was approximately 10.6 N, the maximum (maximum) force was approximately 12.8 N, and the average (maximum) force was approximately 11.2 N with a standard deviation of approximately 0.50 N. The overall average (maximum) force from the multi-day study was approximately 11.0 N with an overall standard deviation of approximately 0.5N.

In some embodiments, the maximum injection force of delivery device 2800 may include an upper tolerance limit of approximately 13.7 N, a maximum injection force of approximately 13.5 N with a standard deviation of approximately 0.4 N, and an intermediate precision including upper tolerance limit of approximately between 13.7 N to 17.5 N, an overall maximum injection force of approximately 15.4 N with a standard deviation of approximately 1.3 N. In some embodiments, the maximum injection force of delivery device 2800 may include an upper tolerance limit of 28.2 N. Upon completion of performing the injection, the user may dispose of any unused medicament or waste material by discarding delivery device 2800. As described above, one or more markings or indicia may be present on one or more components (e.g., body 1220, stopper 1222, actuation portion 2882, and/or flange piece 2870) of delivery device 2800 to assist in determining the status of delivery device 2800. For example, upon plunger rod 2880 being translated by a second distal translation (i.e. the second push) relative to flange piece 2870 and body 1220 for delivering medicament out of expulsion end 1224, delivery device 2800 may be in a delivered and/or completed status. The one or more markings or indicia may be configured to indicate to a user the delivered and/or completed status of delivery device 2800.

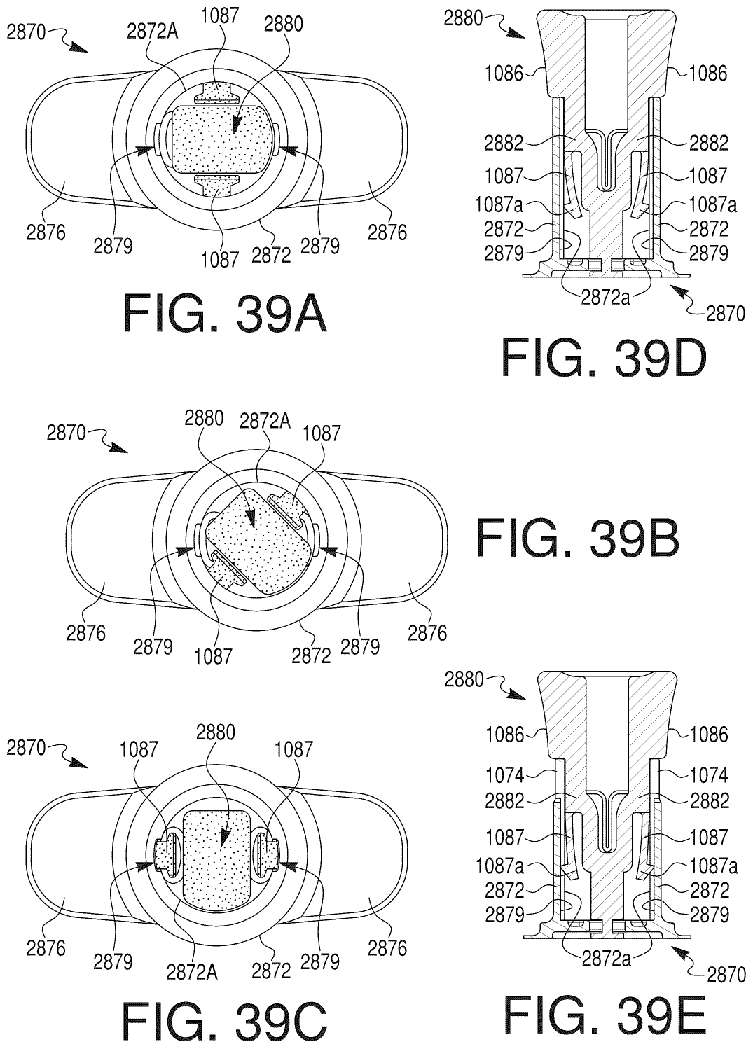

Referring back to FIGS. 39A-39B, extensions 1087 may contact an interior surface 2872A of collar 2872 as plunger rod 2880 rotates relative to flange piece 2870. As seen in FIG. 39D, with hook or clip shaped part 1087a engaged against interior surface 2872A, extensions 1087 may be deflected radially-inward by collar 2872 until plunger rod 2880 is rotated to align extensions 1087 with internal grooves 2879 of flange piece 2870. Internal grooves 2879 may define recesses formed along interior surface 2872A. As seen in FIG. 39C and FIG. 39E, internal grooves 2879 may be sized and shaped to receive extensions 1087 therein. It should be appreciated that collar 2872 may have a greater diameter at internal grooves 2879 than along interior surface 2872A such that extensions 1087 are configured to expand radially-outward from a compressed configuration (FIGS. 39A-39B and FIG. 39D) to an expanded configuration (FIG. 39C and FIG. 39E) when extensions 1087 are moved into radial alignment with internal grooves 2879.

Stated differently, extensions 1087 may be transitioned to a relaxed state when received within internal grooves 2879 due to the additional space provided by internal grooves 2879, as seen in FIG. 39E. In some instances, a feedback (e.g., tactile, auditory, etc.) may be generated in response to extensions 1087 being received within internal grooves 2879. Delivery device 2800 may be positioned in a dosage delivery state such that further actuation of plunger rod 2880 may provide a dose delivery. In some embodiments, flange piece 2870 may be operable to generate a user feedback (e.g., tactile, audible, etc.) upon rotating plunger rod 2880 relative to flange piece 2870 to prime delivery device 2800.

As described in detail above and as seen in FIGS. 40A-40C, opening 1073 may have a semi-circular shape with one or more edges 2871′ extending into opening 1073. With plunger rod 2880 coupled to flange piece 2870, stem 1280 may be received through opening 1073. Stem 1280 may include a rounded sidewall 2884 that is configured to interact with the one or more edges 2871′ as plunger rod 2880 rotates relative to collar 2872. For example, rounded sidewall 2884 may define a semi-circular end along stem 1280 that may contact edges 2871′ when plunger rod 2880 is moved from the primed position (FIG. 38B) to the dosage delivery position (FIG. 38C). As described in detail above (FIGS. 4K-4X), it should be appreciated that stem 1280 may have various suitable shapes and/or configurations for facilitating movement (e.g., rotation) of plunger rod 2880 relative to flange piece 2870.

Referring now to FIG. 38D, with protrusions 1086 aligned with slots 1074, plunger rod 2880 may be translated distally relative to collar 2872 to complete a full stroke of plunger rod 2880 in response to applying a distally-directed force onto actuation portion 2882. Stated differently, plunger rod 2880 may be translated by a final distal translation (i.e. the second push) relative to flange piece 2870 and body 1220 to complete delivery. In this instance, stem 1280 may move relative to flange piece 2870, thereby causing stopper 1222 to move within body 1220 to deliver a dosage. In this instance, protrusions 1086 may be received within slots 1074 and second ring 2889 may be positioned proximate to a proximal end of collar 2872. In other words, in some embodiments, second ring 2889 does not contact the proximal end of collar 2872.

Further, as described in greater detail above, indents 2875 may receive extensions 1087 (FIG. 36) therein when completing a full stroke of plunger rod 2880. It should be appreciated that an extent that plunger rod 2880 translates relative to flange piece 2870 may define a dosage delivery distance of delivery device 2800. The dosage delivery distance may be controlled based on a longitudinal length of protrusions 1086 relative to actuation portion 2882 and/or a depth of slots 1074 relative to collar 2872. Accordingly, it should be appreciated that delivery device 2800 may be configured and operable to perform a series of steps, including priming, transitioning, and delivering the medicament stored therein, in response to a push-twist-push action onto plunger rod 2880 relative to flange piece 2870 and body 1220, or vice versa. The configurations and design of one or more of plunger rod 2880 and/or flange piece 2870 may be configured to prevent the possibility of delivering a volume outside (i.e. less than or greater than) the predefined dose volume intended for delivery from delivery device 2800. Accordingly, delivery device 2800 may be configured to prevent administration of any residual volume remaining in body 1220 after the predefine dose volume is delivered, despite an application of additional force onto plunger rod 2880. Delivery device 2800 may be configured to complete the injection stroke prior to stopper 1222 contacting a distal interior surface/end of body 1220, thereby preventing any potential breakage of body 1220 and/or stopper 1222 due to excessive compression of stopper 1222 by plunger rod 2880.

As seen in FIGS. 41A-41D, delivery device 2800 may include various other flange pieces 2870 having at least one of a plurality of textured interfaces on flanges 2876. As merely an illustrative example, as seen in FIG. 41A, an alternative exemplary flange piece 2870A may include a textured interface 2878A on flanges 2876 comprising a plurality of circular protrusions and/or depressions arranged in an annular array relative to one another. As seen in FIG. 41B, another exemplary flange piece 2870B may include a textured interface 2878B comprising an ornamental design, such as a snowflake, on each flange 2876. FIG. 41C shows an exemplary flange piece 2870C including a textured interface 2878C on flanges 2876 comprising a plurality of circular protrusions and/or depressions arranged in an irregular pattern relative to one another.

By way of further example, referring now to FIG. 41D, a flange piece 2870D may include a textured interface 2878D comprising a plurality of diamond-shaped protrusions and/or apertures positioned in a grid-like arrangement along flanges 2876. It should be understood that the various textured interfaces shown and described herein may be configured to enhance a grip, control, aesthetic, and/or ergonomics of the flange piece. It should further be appreciated that the textured interfaces shown and described herein are merely illustrative such that various other suitable patterns, textures, and/or features may be included on the flange pieces without departing from a scope of this disclosure.

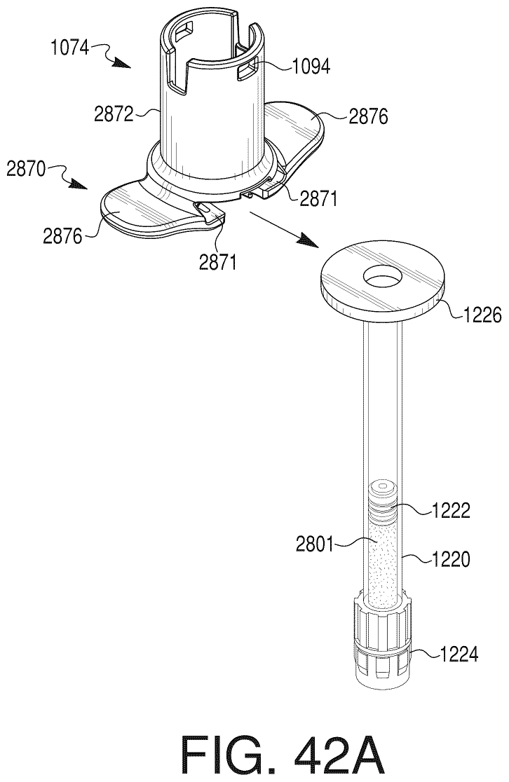

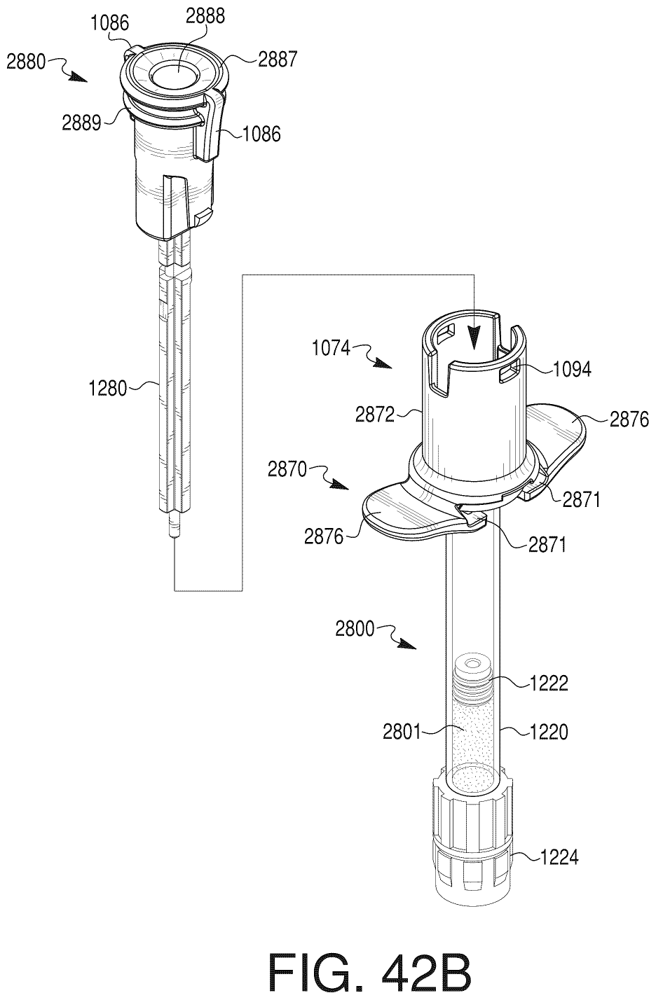

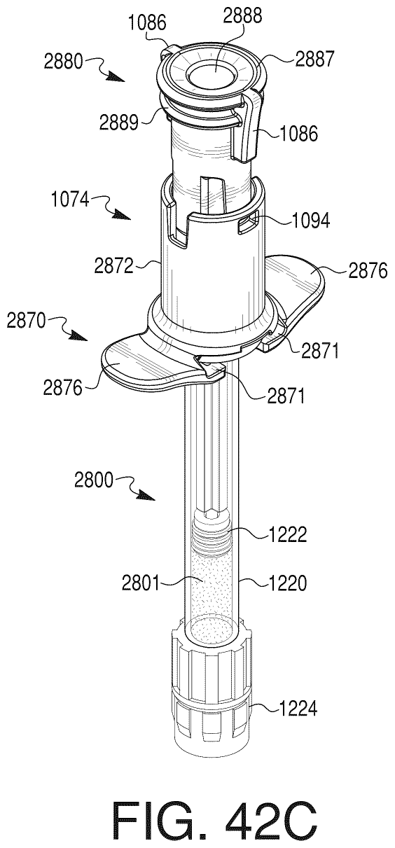

FIGS. 42A-42C depict an exemplary method of assembling delivery device 2800. Referring initially to FIG. 42A, body 1220 and flange piece 2870 may be separate from one another prior to assembly of delivery device 2800. Body 1220 may include body flange 1226 at a proximal end of body 1220 opposite expulsion end 1224 located at a distal end of body 1220. During assembly, body flange 1226 may be inserted over lip 2871 and into a lateral slot positioned below collar 2872 to couple flange piece 2870 to body 1220.

In the example, expulsion end 1224 may include a cap coupled to body 1220 such that the cap may be preassembled onto body 1220. Body 1220 may be filled with a substance 2801 prior to use of delivery device 2800, such that delivery device 2800 may be prefilled upon assembly. Substance 2801 may include a predetermined amount or dose of fluid. For example, substance 2801 may be one of a drug, a medicament, or similar therapeutic substance as described in detail herein (e.g., a VEGF antagonist). Stopper 1222 may be inserted into body 1220 through a proximal opening defined in body flange 1226 to fluidly seal substance 2801 therein. In this instance, body 1220 may be configured to hold substance 2801 between expulsion end 1224 and stopper 1222 to maintain a sterility of substance 2801. Stopper may have a height in the range from about 28.0 mm to 32.0 mm; in the range from about 29.0 mm to 31.0 mm; and/or be approximately 31.0 mm.

In some embodiments, stopper 1222 may be inserted into body 1220 within a relatively short period of time after filling substance 2801 therein to limit the exposure and/or likelihood of contamination of substance 2801 within body 1220, such as within a minute or less. Reducing a time between filling body 1220 with substance 2801 and subsequently inserting stopper 1222 may reduce the effects of potential exposure and/or contamination of substance 2801 within body 1220. In some examples, body 1220 may be filled with substance 2801 by a pump, such as a peristaltic pump. Body 1220 may be filled with substance 2801 to a target weight in the range of approximately 0.175 g to 0.310 g; such as in the range from approximately 0.175 g to 0.215 g; and/or 0.250 g to 0.310 g. In some embodiments, the target weight of the substance is about 0.190 g, about 0.195 g, or about 0.200 g. In some embodiments, body 1220 may be filled with substance 2801 to a target fill volume in the range of approximately 0.16 mL to 0.20 mL; such as 0.18 mL. In some embodiments, body 1220 may have a maximum fill volume of substance 2801 of approximately 0.23 mL. In some embodiments, body 1220 may be sized and/or filled with substance 2801 such that there may be a space between substance 2801 and stopper 1222 defining a headspace volume. In some embodiments, the headspace volume may be in the range from approximately 0.03 mL to approximately 0.07 mL. In some embodiments, the headspace may be approximately 0.03 mL; approximately 0.05; and/or approximately 0.07 mL based on the target fill volume of substance 2801 filled within body 122. In some embodiments, the process of inserting stopper 1222 into body 1220 may be performed via a vacuum chamber. Stated differently, a vacuum pressure may be applied during the placement of stopper 1222 into body 1220 to effectively form a sterile seal against substance 2801 stored therein. After body 1220 is filled with substance 2801, body 1220 may be sealed, such as, for example, by crimping. For example, body 1220 may be sealed with a crimping pressure of approximately 3.0 bar. After body 1220 is crimped, substance 2801 may be stored at a temperature in the range from approximately 2-8° C. As described herein, delivery device 2800 may be provided in a pre-primed configuration, thereby requiring an initial priming during use of delivery device 2800 prior to delivering of the predetermined amount or dose of substance 2801 to a user (e.g., a patient).

Still referring to FIG. 42A, flange piece 2870 may be attached to body 1220 by receiving body flange 1226 within the lateral slot of flange piece 2870 defined between collar 2872 and lip 2871. It should be appreciated that flange piece 2870 may be coupled to body 1220 prior to inserting plunger rod 2880 in body 1220. A width and/or diameter of the lateral slot may correspond to the width and/or diameter of body flange 1226, and may be defined between the pair of flanges 2876. Lip 2871 may be configured to hold body flange 1226 in place by slidably coupling flange piece 2870 to body 1220. The one or more ribs 2874 (see FIG. 36) of flange piece 2870 may be configured to engage body flange 1226 of body 1220 when received within the lateral slot and coupled to flange piece 2870. Ribs 2874 may be positioned adjacent to lip 2871 and the lateral slot of flange piece 2870 may be sized, shaped, and/or otherwise configured to receive body flange 1226 therethrough. Ribs 2874 may be configured to secure a position (e.g., longitudinal, rotational, etc.) of body 1220 relative to flange piece 2870 by engaging a proximal surface of body flange 1226 as lip 2871 engages a bottom surface of body flange 1226.

In some embodiments, when lip 2871 engages body flange 1226, a space and/or gap may be formed between one or more portions of body 1220 and flange piece 2870 due to the connection interface therebetween. In this instance, atmospheric air and/or an airflow of a sterilization agent may be permitted to flow through the space and/or gap, such as to facilitate sterilization of delivery device 2800 upon assembly and prior to use. In some embodiments, flange piece 2870 may include additional features for engaging, contacting, and/or separating a surface of flange piece 2870 from an adjacent surface of body 1220 when coupled to one another, thereby forming a space and/or gap therebetween to facilitate airflow therebetween. For example, as best seen in FIGS. 41A-41D, flange piece 2870 may include an abutment 2869 (e.g., a protrusion, a bump, a projection, a tab) that may be sized, shaped, and/or otherwise configured to contact, abut against, and/or engage a portion of body 1220 to separate an interior surface of flange piece 2870 from an exterior surface of body 1220. In some embodiments, devices according to the present disclosure may be evaluated during a step in which they are labeled and assembled as described in detail above. For example, assembly of flange piece 2870 may withstand a force of up to approximately 65 N when attached to body 1220. The upper limit of the finger flange assembly force may be based on design characterization studies demonstrating that the process range is non-impactful to product quality attribute for the drug substance stored in body 1220.

Referring now to FIG. 42B, plunger rod 2880 may be inserted into body 1220 and through flange piece 2870. It should be appreciated that plunger rod 2880 may be configured for assembly with body 1220 after flange piece 2870 is coupled to body 1220. Plunger rod 2880 may be inserted through a top opening of collar 2872 until stem 1280 and a distal end of plunger rod 2880 is received within body 1220. In some embodiments, assembly of plunger rod 1080 may withstand a force of up to approximately 15 N when inserted into flange piece 2870. Plunger rod insertion force may be connected to primary container closure integrity (CCI) as the process parameter may have the potential to impact a position of stopper 1222, which determines the location of the sterile boundary in delivery device 2800. Development studies measured the forces generated during the insertion of plunger rod 1080 into body 1220 to confirm that a 15 N force is acceptable for assembly. Study results show no impact on CCI or deliverable volume with a 15 N insertion force limit.

In some embodiments, the distal end of plunger rod 2880 may become attached to stopper 1222 within body 1220 during assembly. In further embodiments, the distal end of plunger rod 2880 may contact but not become securely attached to stopper 1222 during assembly, such that plunger rod 2880 may be configured to move stopper 1222 distally within body 1220 in response to plunger rod 2880 moving distally, and not entirely remove and/or retract stopper 1222 proximally from out of body 1220 in response to plunger rod 2880 moving proximally. As seen in FIG. 42C, plunger rod 2880 may be securely coupled to flange piece 2870 in an initial position relative to body 1220 upon extensions 1087 being received within side openings 1094, and particularly hook or clip shaped parts 1087a extending into side openings 1094, as described in detail above. In this instance, delivery device 2800 may be in an assembled state and a pre-primed configuration prior to use. As described further below, delivery device 2800 may be packaged and sterilized prior to use by a user for delivering a deliverable volume of the drug substance/product pre-filled therein.

The deliverable volume (or delivered volume) is the volume of the drug product expelled from the prefilled syringe (delivery device 2800) assembled with a finger flange (flange piece 2870) and plunger rod device (plunger rod 2880) after priming. Deliverable volume is measured at prefilled syringe release and end-of-shelf-life to ensure that the correct dosage of the drug is administered to the patient. The prefilled syringe (e.g., delivery device 2800) may be designed to provide a single dose through a deliverable volume of 70 L≤x<100 μL. The lower end of the acceptance criteria ensures that the device consistently delivers a minimum labeled dose volume of 70 μL (8 mg). The upper acceptance criteria prevent the possibility of delivering a volume of 100 μL or greater, as too high of an injection volume (>100 μL) may contribute to adverse effects such as transiently increased intraocular pressure. The fill volume of the devices of the present disclosure (e.g., delivery device 2800) may be established through consideration of prime (i.e. volume required to expel air bubbles from the device), dose (intended volume to be delivered to the patient from the device), and residual (amount of drug product remaining in the device following dose delivery). The devices of the present disclosure may be designed to consistently deliver a single minimum labeled dose, such as, for example, 70 μL (8 mg) of a drug substance/product. In other embodiments, the deliverable volume from the devices of the present disclosure (e.g., delivery device 2800), such as a pre-filled silicone-free syringe, may be less than approximately 250 μL, such as less than approximately 200 μL, such as less than approximately 100 μL, such as less than approximately 90 L. Fill weight may be controlled as part of the drug product manufacturing control strategy and may be considered a critical process parameter.

Components of the devices described herein may be designed and/or suited for manufacture in one or more ways. In some embodiments, for example, components of the devices described herein (e.g., device 1050, device 1200, device 1300, device 1400, device 2400, device 2500, device 2600, device 2800, etc.) may be suitable for manufacture via, e.g., injection molding, 3-dimensional printing, or machining. In one embodiment, for example, components of the devices may be particularly suited for manufacture via injection molding. For example, in some existing devices, molding is not suitable for high volume production, resulting in the use of 3-dimensional printing. In some embodiments, while manufacturing tolerances may be tighter with molding techniques than with 3-dimensional printing techniques, devices formed by 3-dimensional printing do not have the same level of precision as devices formed by molding. Precision may be particularly important for devices of the present disclosure, for example, those devices used for vitreous injections at volumes of 100 μL or less. In some embodiments, the intraday precision for devices of the present disclosure may include a minimum of about 0.083 mL, a maximum of about 0.087 mL, a mean of about 0.085 mL with a standard deviation of 0.001 mL, and an intermediate precision including a minimum of about 0.071 mL, a maximum of about 0.087 mL, a mean of about 0.085 mL with a standard deviation of 0.002 mL.

For example, an exemplary study was performed using approximately 30 syringes to determine an intra-day precision (repeatability) and approximately 120 syringes to determine an intermediate precision for deliverable volume from a 0.5 mL pre-filled syringe. The intra-day precision study resulted in a minimum delivered volume of approximately 0.083 mL, a maximum delivered volume of approximately 0.087 mL, and an average delivered volume of approximately 0.085 mL with a standard deviation of approximately 0.001 mL. The intermediate precision study resulted in a minimum delivered volume of approximately 0.071 mL, a maximum delivered volume of approximately 0.087 mL, and an average delivered volume of approximately 0.085 mL with a standard deviation of approximately 0.002 mL. It should be appreciated that for each of the intra-day and intermediate precision studies, the depth of the channel of the pre-filled syringe body may affect or dictate the deliverable volume (e.g., standard deviation may vary).

Accordingly, it should be appreciated that devices of the present disclosure described herein may be designed to store predefined volumes of therapeutic agent that may be suitable for vitreous (IVT) injections, such as, for example, 100 μL or less. In some embodiments, the devices described herein may be designed for injection of certain volumes of vitreous (e.g., 0.5 mL, 1.0 mL, etc.) based on an intended use of the device in a particular procedure. In some embodiments, the volume of vitreous may refer to a volume stored within the device or a volume delivered to the patient. For example, devices of the present disclosure may be configured to store a volume of vitreous of about 65 μL to about 75 μL for high dose aflibercept procedures; about 95 μL to about 105 μL for Mini Trap procedures; and/or about 5 μL to about 15 μL for Retinopathy of Prematurity (ROP).

Devices of the present disclosure may be further configured to store relatively greater volumes of vitreous for injection based on a degree of myopia, such as about 3 milliliters, 4 milliliters, and greater. Additionally, the devices described herein may be designed for injection of larger volumes of vitreous based on an intended procedure, such as, about 3 ml to about 6 ml of silicone or gas for tamponade post vitrectomy. In some embodiments, devices of the present disclosure may be configured to deliver a dosage volume of vitreous that is relatively less than a storage volume of the vitreous that is prefilled in the device, such as from about 0.5 mL to 1.0 mL. It should be appreciated that the devices of the present disclosure may be designed to inject various other volumes of vitreous relative to other procedures, such as, Diabetic Eye Disease, post-injection noninfectious Endophthalmitis, Neovascular (Wet) Age-related Macular Degeneration (AMD), Macular Edema following Retinal Vein Occlusion (RVO), Diabetic Macular Edema (DME), and Diabetic Retinopathy (DR).

### Methods of Treatment & Administration of Medicament

For some products in particular, e.g., ophthalmic, vaccinations, insulin, or other drug products, dose accuracy may be particularly important. However, it is also contemplated that embodiments of the present disclosure may be applicable to any other liquid products or any other context for which precise methods for setting and administering a reliably accurate dose or delivery volume are beneficial.

For example, devices and aspects of the present disclosure may provide methods as set forth herein wherein a VEGF antagonist (e.g., aflibercept) is delivered with a high amount of precision, e.g., with a drug delivery device (DDD) (e.g., with a 0.5 mL volume), whether pre-filled or capable of being filled from a vial, and delivering a volume of between 70 and 100 microliter with an average volume of about 81 or 82 or 81-82 microliters, e.g., with a standard deviation of about 4 or 5 or 4-5 microliters (e.g., about 4.5 or 4.46 microliters) or less. In some embodiments, the DDD is a syringe, e.g., with a 30 gauge, ½ inch needle. In further embodiments, a dosage volume to be delivered from the DDD may range from about 0.5 mL to 1.0 mL. Stated differently, the DDD dosage volume may range from about 10 microliters to about 100 microliters. In some embodiments, such as when delivering a High dose of Eylea (e.g. 8 mg of aflibercept), as described in more detail below, the DDD may include a dose volume of about 70 microliters. In some embodiments, such as when delivering a Standard dose of Eylea (e.g., 2 mg of aflibercept), as described in more detail below, the DDD may include a dose volume of about 50 microliters, with the concentration of the Standard dose being relatively less than the High dose.

One means for ensuring precision of a dose to be delivered with a device, such as a syringe, is by employing a syringe wherein the dose volume is device-determined. If the dose volume is device-determined, the device is designed only to deliver a single volume (e.g., 87 microliters) or a single volume with a limited amount of acceptable error (+4-5 microliters). Thus, if used properly, the user cannot deliver the wrong dose (e.g., cannot deliver more than the intended volume from the device).

The present disclosure includes embodiments wherein, a precise dosage of about 8 mg or more is a dose of about 9, 9.3, 9.33, 9.7, 9.8, 9.9, 9.7-9.9 mg or more + about 0.5, or + about 0.51 mg is delivered to a subject's eye. The volume in which a dose is delivered can be, for example, about 70, 81, 82, 81.7, 85, 86, 87, 85-87 microliters+ about 4, 4.45, 4.5, or 5 microliters. In further examples, the volume in which a dose is to be delivered (i.e. a dosage volume) can be about 0.5 milliliters or 1.0 milliliters. Doses may be delivered with a dose delivery device (DDD) which is a syringe.

Highly precise doses of VEGF antagonist (e.g., aflibercept) may be delivered, for example, in a volume that is device-determined (wherein the device is a syringe), by a method that includes the steps: (a) priming the syringe (e.g., a pre-filled syringe), thereby removing air from the syringe and, thus avoiding injection of air into the eye, by advancing the plunger rod by a predetermined distance into the syringe body until advancement of the plunger rod is resisted by a stop; (b) rotating the plunger rod about a longitudinal axis; and (c) actuating the plunger rod to dispense a predetermined (device-determined) volume (e.g., about 70, 81, 82, 81.7, 85, 86, 87, 85-87 microliters, + about 4, 4.45, 4.5, or 5 microliters) of the formulation.

In some embodiments, a precise dosage of about 8 mg or more is a dose of about 9, 9.3, 9.33, 9.7, 9.8, 9.9, 9.7-9.9 mg or more + about 0.5, or + about 0.51 mg is delivered to a subject's eye. The volume in which a dose is delivered can be, for example, about 70, 81, 82, 81.7, 85, 86, 87, 85-87 microliters + about 4, 4.45, 4.5, or 5 microliters. Doses may be delivered with a dose delivery device (DDD) which is a syringe, exemplary embodiments of which are described herein.

Increasing the molar fraction of VEGF antagonist therapeutic protein in the dosing formulation is a potential way to bring further benefits to patients with chorioretinal vascular diseases, including nAMD. A higher dose of aflibercept administered IVT has the potential to prolong the drug's therapeutic effects and for improvement in pharmacodynamics such as better drying. The resulting extension of treatment intervals early after the initiation of treatment to every 12 weeks would reduce the number of injections in the first treatment year. A potential decrease in injection-related treatment burden and safety events with fewer injections could be a significant contribution to patient care and healthcare services. The present invention provides, in part, a safe and effective method for treating angiogenic eye disorders with an 8 mg dose of aflibercept in a regimen calling for monthly loading doses before quarterly maintenance doses.

The ophthalmic formulation for a standard dose and a high dose of aflibercept (e.g., Eylea) may include one or more of the following characteristics and/or properties shown in the table below.

| Standard Dose Eylea | High Dose Eylea |
|---|---|
| 2 mg Aflibercept | 8 mg Aflibercept |
| 10 mM Sodium Phosphate | 50 mM L-arginine |
| | monohydrochloride |
| 40 mM Sodium Chloride | 10 mM histidine-based buffer |
| 0.03% Polysorbate 20 | 0.03% Polysorbate 20 |
| 5% Sucrose | 5% Sucrose |
| pH 6.2 | pH 5.8 |
| (Delivered in a volume of 50 μL) | (Delivered in a volume of 70 μL) |

In at least the High Dose embodiment, it should be understood that the L-arginine monohydrochloride may include a viscosity reducing agent and a “thermal stabilizer;” the histidine-based buffer may include a “buffer;” the Polysorbate 20 may include a “surfactant;” the Sucrose may include a “thermal stabilizer;” the Sodium Phosphate may include a “buffer;” and the Sodium Chloride may include a “viscosity reducing agent.” In some embodiments, the appearance of the medicament of the standard dose and the high dose may be essentially free from visible particulates. In some embodiments, the appearance of the medicament of the standard dose and the high does may be opalescent. In some embodiments, the particulate matter by Malvern may be greater than or equal to 10 μm: 1-3 particles/mL, greater than or equal to 25 μm: 0-1 particles/mL, or greater than or equal to 50 μm: 0 particles/mL.

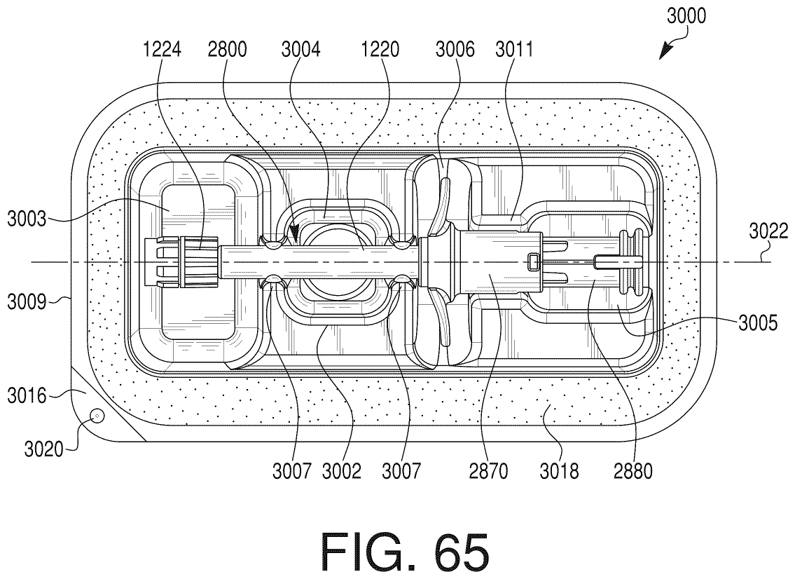

In some embodiments, the medicament may be evaluated as a formulated drug substance (FDS) by one or more methods described in further detail herein, such as including, but not limited to, physical form/condition; clarity; color; pH; total protein content; identity by dot blot; potency by bioassay; purity by reduced capillary electrophoresis sodium dodecyl sulfate (CE-SDS); purity by non-reduced capillary electrophoresis sodium dodecyl sulfate (CE-SDS); purity by size-exclusion ultra high performance liquid chromatography (SE-UPLC); charge variant analysis by imaging capillary isoelectric focusing; and/or polysorbate 20. In other embodiments, the medicament may be evaluated as a drug product (DP) by one or more methods described in further detail herein, such as including, but not limited to, isoaspartate assay; endotoxin content; bioburden; and sterility of the filled container. As described above, the present invention provides a method for treating angiogenic eye disorders in a regiment calling for monthly loading doses. First, patients receiving an 8 mg dose, which is four times the dose approved for Eylea, (2 mg), were not observed in the CANDELA clinical trial (discussed herein) to develop any more hypertension treatment-emergent adverse events than patients receiving 2 mg (FIG. 65). Systemic exposure to VEGF inhibitor is known to be associated with the risk of systemic blood pressure (BP) increases. Moreover, intravitreal injection of VEGF inhibitors has been associated with blood pressure effects. See, for example, Hanna et al., (2019) “Three patients with injection of intravitreal vascular endothelial growth factor inhibitors and subsequent exacerbation of chronic proteinuria and hypertension”, Clinical Kidney Journal, 2019, vol. 12, no. 1, 92-100 and Raiser et al., (2009) “The effect of intravitreal bevacizumab (Avastin) administration on systemic hypertension”, Eye 23, 1714-1718.

In addition, in the CANDELA clinical trial, subjects receiving the 8 mg dose (HD) achieved numerically superior anatomical improvements in the eye as well as numerically superior improvements in vision relative to subjects receiving a 2 mg dose (IAI). A higher proportion of eyes treated with aflibercept 8 mg (HD) were dry (without intraretinal or subretinal fluid on OCT) in the center subfield versus aflibercept 2 mg (IAI). Treatment groups followed identical dosing regimens with the 8 mg group receiving slightly fewer PRN doses. A change from baseline in central retinal thickness (CRT) suggested better anatomic outcomes with aflibercept 8 mg versus aflibercept 2 mg; and a change from baseline in BCVA favored aflibercept 8 mg (+7.9 vs+5.1 letters).

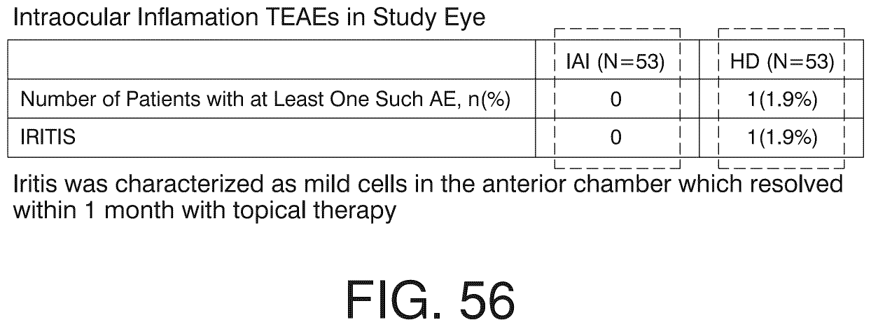

The anatomical and visual improvements for the HD patients that was observed was also comparable to those of subjects in the VIEW1 and VIEW2 trials (VIEW1/2) that received a 2 mg dose every 8 weeks (following three monthly loading doses (2q8)). See e.g., Heier et al., Intravitreal Aflibercept (VEGF Trap-Eye) in Wet Age-related Macular Degeneration, Ophthalmology 2012; 119:2537-2548. The VIEW1/2 2q8 subjects achieved 8.1 letters of BCVA mean improvement at 44 weeks following the initial dose (Heier et al. (2012), FIG. 3); whereas the CANDELA HD subjects herein were observed to achieve a mean improvement of 7.9 letters at 44 weeks (FIG. 56).

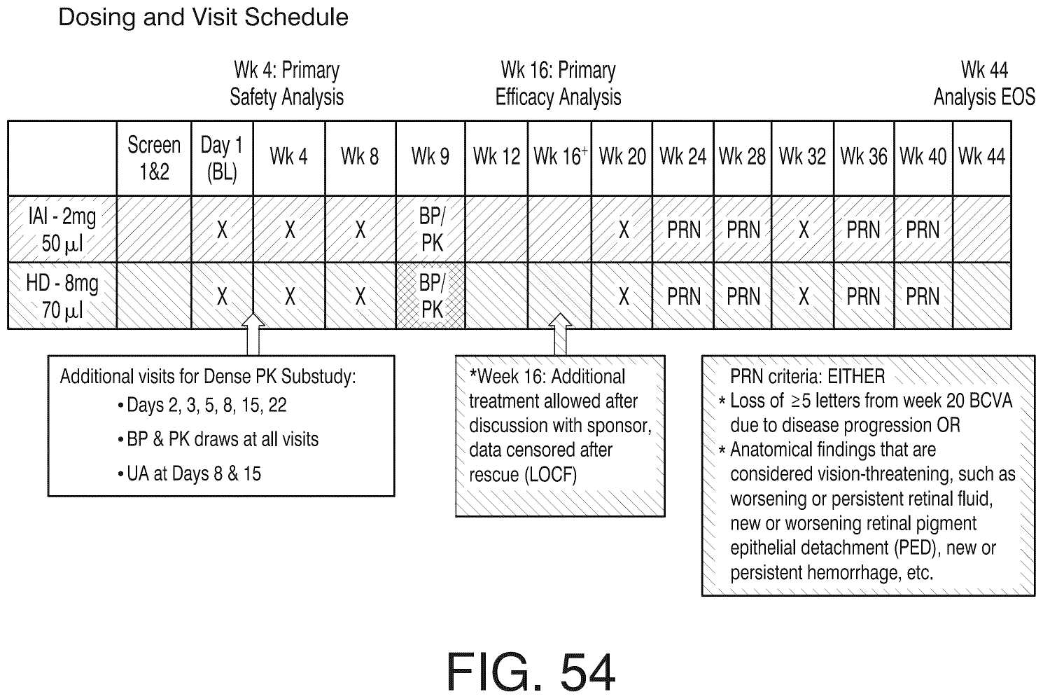

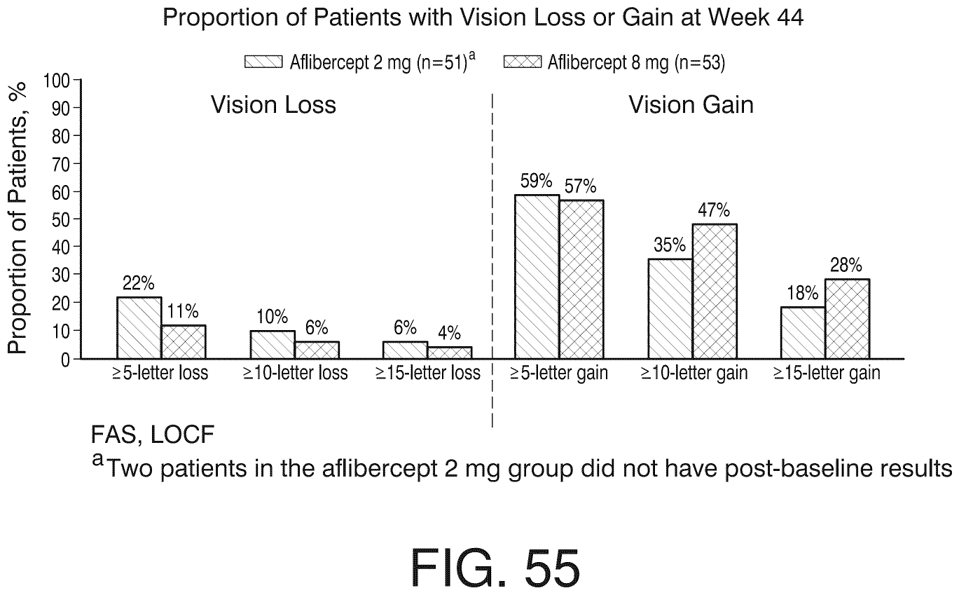

Moreover, the improvements in the central retinal thickness (CRT) that were observed to be achieved by CANDELA HD subjects were comparable to that observed in the VIEW1/2 2q8 subjects. The VIEW1 2q8 subjects achieved a mean reduction of about 125 micrometers in CRT and VIEW2 2q8 subjects achieved about 150 micrometers mean reduction in CRT, at 44 weeks (Heier et al. (2012), FIG. 4). The CANDELA HD subjects were observed to achieve a mean reduction of 159 micrometers and a median reduction of 162 micrometers at 44 weeks (FIG. 54 & FIG. 55; and Heier et al. (2012), FIG. 4).

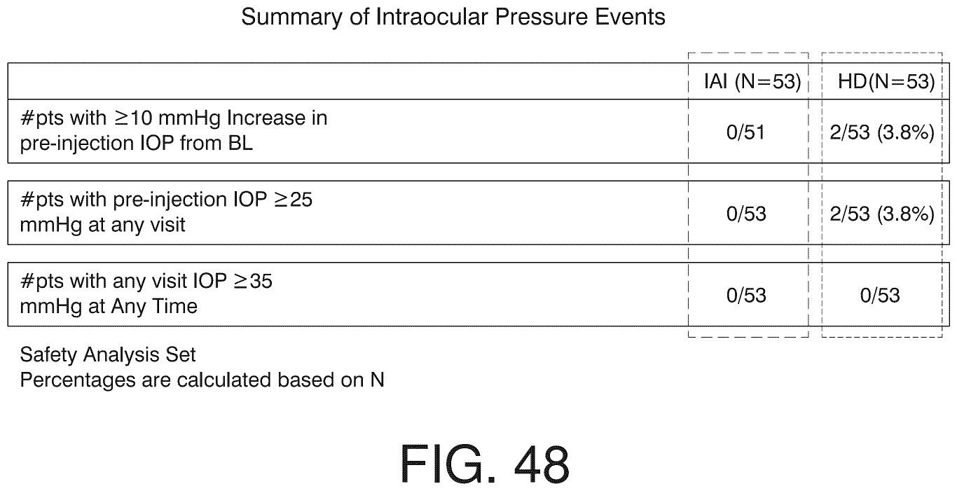

While the 2q8 VIEW1/2 subjects and the CANDELA HD subjects were not evaluated side-by-side in the same clinical trial and the VIEW trials had a greater number of participants, these data suggest that subjects can be administered 8 mg doses of aflibercept as infrequently as every 12 weeks yet achieve anatomic and visual outcomes comparable to that of patients dosed every 8 weeks (following three monthly loading doses) with 2 mg. While the VIEW1/2 subjects received only the scheduled doses within the first year, some of the CANDELA subjects received additional doses pro re nata after week 16 (FIG. 1). Twenty eight out of 53 of the CANDELA HD subjects did not receive any additional doses (FIG. 48). Moreover, time-domain optical coherence tomography was performed in VIEW1/2 to evaluated CRT whereas, a more sensitive spectral domain optical coherence tomography (SD-OCT) was used in CANDELA.

In further embodiments of methods, preparatory steps may include use of one single-dose glass vial having a protective plastic cap and a stopper containing an aqueous formulation comprising >8 mg aflibercept in about 70 microliters; one 18-gauge×1½-inch, 5-micron, filter needle that includes a tip and a bevel; one 30-gauge×½-inch injection needle; and one 1-mL Luer lock syringe having a graduation line marking for 70 microliters of volume; packaged together (kits including such items form part of the present invention).

The steps can include, for example: (1) visually inspecting the aqueous formulation in the vial and, if particulates, cloudiness, or discoloration are visible, then using another vial of aqueous formulation containing the aflibercept; (2) removing the protective plastic cap from the vial; and (3) cleaning the top of the vial with an alcohol wipe; then, using aseptic technique the following steps: (4) removing the 18-gauge×1½-inch, 5-micron, filter needle and the 1 mL syringe from their packaging; (5) attaching the filter needle to the syringe by twisting it onto the Luer lock syringe tip; (6) pushing the filter needle into the center of the vial stopper until the needle is completely inserted into the vial and the tip touches the bottom or a bottom edge of the vial; (7) withdrawing all of the aflibercept vial contents into the syringe, keeping the vial in an upright position, slightly inclined, while ensuring the bevel of the filter needle is submerged into the liquid; (8) continuing to tilt the vial during withdrawal keeping the bevel of the filter needle submerged in the formulation; (9) drawing the plunger rod sufficiently back when emptying the vial in order to completely empty the filter needle; (10) removing the filter needle from the syringe and disposing of the filter needle; (11) removing the 30-gauge×½-inch injection needle from its packaging and attaching the injection needle to the syringe by firmly twisting the injection needle onto the Luer lock syringe tip; (12) holding the syringe with the needle pointing up, and checking the syringe for bubbles, wherein if there are bubbles, gently tapping the syringe with a finger until the bubbles rise to the top; and (13) slowly depressing the plunger so that the plunger tip aligns with the graduation line that marks 70 microliters on the syringe.

Preferably, injection of a VEGF antagonist (e.g., a VEGF receptor fusion protein, such as aflibercept) may be performed via the methods of the present disclosure under controlled aseptic conditions. For example, such conditions may be obtained through surgical hand disinfection, the use of sterile gloves, a sterile drape, and/or a sterile eyelid speculum (or equivalent thereof). Additionally, anesthesia and/or a topical broad-spectrum microbicide may be administered to the patient prior to administration of the VEGF antagonist through the injection.

The present disclosure further provides methods for treating angiogenic eye disorders (e.g., DR and/or DME) by sequentially administering initial loading doses (e.g., 2 mg or more, 4 mg or more, preferably, about 8 mg or more of VEGF antagonist or inhibitor, for example a VEGF receptor fusion protein such as aflibercept) (e.g., about every 2-4 or 3-5 weeks) followed by additional doses every 12-20 weeks, preferably 12-16 weeks, 12 weeks, 16 weeks or 20 weeks. For example, in an embodiment of the invention, the methods of the present invention include treating or preventing angiogenic eye disorders, such as diabetic retinopathy (DR), diabetic macular edema (DME) or neovascular AMD, by administering, sequentially, one or more (e.g., 3, 4 or 5) doses of about 8 mg or more of VEGF antagonist (e.g., a VEGF receptor fusion protein such as aflibercept) about every 2-4 or 3-5 weeks, e.g., every month (or about every 28 days, 28±5 days or about every 4 weeks), followed by one or more doses of about 8 mg or more VEGF antagonist (e.g., a VEGF receptor fusion protein such as aflibercept) every 12 weeks (or about every 3 months or about every quarter year or about every 84 days) or every 16 weeks (or about every 4 months or about every 1/3 years or about every 112 days) or every 20 weeks. For example, in an embodiment of the invention, about 8 mg is 7.2 mg, 8.8 mg or 7.2-8.8 mg or 8 mg+ about 10%. The dosing regimen including the 12 week tertiary dosing interval may be referred to herein as a 12 week dosing regimen or 8q12 or HDq12; the dosing regimen including the 16 week tertiary dosing interval may be referred to herein as a 16 week dosing regimen or 8q16 or HDq16; and the dosing regimen including the 20 week tertiary dosing interval may be referred to herein as a 20 week dosing regimen or 8q20 or HDq20.

In addition, the present invention includes methods for treating angiogenic eye disorders (e.g., DR and/or DME) by administering, one or more times, ≥8 mg VEGF receptor fusion protein, preferably aflibercept, every 4 weeks, 8 weeks, 12-20 weeks, 12-16 weeks, 12 weeks or 16 weeks; as well as every 4 weeks for the first 3, 4 or 5 doses followed by dosing about every 8 weeks. It should be appreciated that the dosing treatment schedule may be based on the indication, such as AMD, DME, DR, or RVO. For example, AMD, DME, and DR require several initial doses on a monthly basis before maintaining on a longer dosing schedule (e.g., every 8 weeks).

In an embodiment of the invention, a subject begins receiving the ≥8 mg maintenance doses of every 12 or 16 or 20 weeks after the 8 mg monthly loading doses with no intervening doses. The subject enters the maintenance dose phase rapidly/immediately after the loading dose phase. In an embodiment of the invention, the subject continues receiving the ≥8 mg 12 or 16 or 20 week doses without any intervening doses.

In an embodiment of the invention, the subject does not receive a dosing regimen modification (DRM) or does not terminate treatment for at least 1, 2, 3, 4 or 5 years. In some embodiments, the subject may be pretreated with one or more different VEGF antagonist from the VEGF antagonist to be later administrated to the subject, such as Eylea. In some embodiments, the subject may be pretreated with the same or substantially the same VEGF antagonist to be later administrated to the subject. In some embodiments, a target location for an intravitreal injection may include the pars plana of the subject's eye. In some embodiments, the process for injection may include applying an iodine solution to sterilize the subject's eye, a topical anesthesia, and injecting the medicament (e.g., Eylea) into the subject's eye at the target location. It should be appreciated that no particular angle is required or necessary when injecting the medicament from the device (e.g., delivery device 2800) to the eye. The medicament may be administered into the intravitreal space of the subject's eye, and guidelines in using the device may be provided for avoiding damaging the lens.

The present invention also provides methods for improving visual acuity in subjects with type 1 or type 2 diabetes mellitus (e.g., subjects with diabetic macular edema or diabetic retinopathy), by administering, sequentially, one or more (e.g., 3 or 4 or 5) doses about every month (or about every 28 days, 28±5 days or about every 4 weeks), followed by one or more doses every 12 weeks (or about every 3 months or about every quarter year or about every 84 days) or every 16 weeks (or about every 4 months or about every 1/3 years or about every 112 days) or every 20 weeks.

The terms “initial dose,” “secondary doses,” and “tertiary doses,” refer to the temporal sequence of administration of the VEGF antagonist (e.g., a VEGF receptor fusion protein such as aflibercept). Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial dose occurs on day 1 for the purposes of counting or numbering days thereafter. The initial, secondary, and tertiary doses may all contain the same amount of VEGF antagonist (e.g., a VEGF receptor fusion protein such as aflibercept), but will generally differ from one another in terms of frequency of administration. In certain embodiments, however, the amount of VEGF antagonist (e.g., a VEGF receptor fusion protein such as aflibercept) contained in the initial, secondary and/or tertiary doses will vary from one another (e.g., adjusted up or down as appropriate) during the course of treatment.

Thus, a dosing regimen of the present invention may be expressed as follows:
a method for treating an angiogenic eye disorder (e.g., DME or DR) in a subject in need thereof including administering (e.g., intravitreally) to the subject in need thereof,
a single initial dose of about ≥8 mg (for example, in about 100 μl or less, about 75 μl or less or about 70 μl or less, e.g., about 50 μl; 51 μl; 52 μl; 53 μl; 54 μl; 55 μl; 56 μl; 57 μl; 58 μl; 59 μl; 60 μl; 61 μl; 62 μl; 63 μl; 64 μl; 65 μl; 66 μl; 67 μl; 68 μl; 69 μl; 70 μl; 71 μl; 72 μl; 73 μl; 74 μl; 75 μl; 76 μl; 77 μl; 78 μl; 79 μl; 80 μl; 81 μl; 82 μl; 83 μl; 84 μl; 85 μl; 86 μl; 87 μl; 88 μl; 89 μl; 90 μl; 91 μl; 92 μl; 93 μl; 94 μl; 95 μl; 96 μl; 97 μl; 98 μl; 99 μl; or 100 μl) of a VEGF antagonist (e.g., a VEGF receptor fusion protein such as aflibercept), followed by
one or more (e.g., 2, or 3 or 4, preferably 2) secondary doses of the VEGF antagonist (e.g., a VEGF receptor fusion protein such as aflibercept), followed by
one or more tertiary doses of the VEGF antagonist (e.g., a VEGF receptor fusion protein such as aflibercept);
wherein each secondary dose is administered 2 to 4 weeks (preferably, about 4 weeks) after the immediately preceding dose; and wherein each tertiary dose is administered 12 or 16 or 20 weeks (preferably, about 12-16, 12 or 16 or 20 weeks) after the immediately preceding dose.

Alternatively, a dosing regimen of the present invention may be expressed as follows:
a single initial dose (e.g., > about 8 mg) of a VEGF antagonist (e.g., aflibercept), followed by
one or more (e.g., 2, or 3 or 4) secondary doses of the VEGF antagonist, followed by
one or more tertiary doses of the VEGF antagonist;
wherein each secondary dose is administered 2 to 4 weeks after the immediately preceding dose; and
wherein each tertiary dose is administered about 12 or 16 or 20 weeks (preferably, about 4 weeks) after the immediately preceding dose; and
wherein each tertiary dose is administered 12 or 16 or 20 weeks (preferably, about 12-16, 12 or 16 or 20 weeks) after the immediately preceding dose. The initial and secondary doses administered before the tertiary doses may be referred to, generally, as “loading” doses. The tertiary doses may be referred to as “maintenance” doses.

The present invention includes methods wherein one or more additional, non-scheduled, pro re nata (PRN) doses, in addition to any of the scheduled initial, secondary and/or tertiary doses of VEGF antagonist (e.g., aflibercept) are administered to a subject. Such PRN doses are typically administered at the discretion of the treating physician depending on the particular needs of the subject.

Additionally and/or alternatively, the present invention includes methods wherein one or more additional, non-scheduled doses, in addition to any of the scheduled initial, secondary and/or tertiary doses of VEGF antagonist (e.g., a VEGF receptor fusion protein such as aflibercept) are administered to a subject. Such doses are typically administered at the discretion of the treating physician depending on the particular needs of the subject. The present invention also provides methods for treating angiogenic eye disorders (e.g., DR or DME) by administering to a subject in need thereof about ≥8 mg (for example, in about 100 μl or less, about 75 μl or less or about 70 μl or less, e.g., about 50 μl; 51 μl; 52 μl; 53 μl; 54 μl; 55 μl; 56 μl; 57 μl; 58 μl; 59 μl; 60 μl; 61 μl; 62 μl; 63 μl; 64 μl; 65 μl; 66 μl; 67 μl; 68 μl; 69 μl; 70 μl; 71 μl; 72 μl; 73 μl; 74 μl; 75 μl; 76 μl; 77 μl; 78 μl; 79 μl; 80 μl; 81 μl; 82 μl; 83 μl; 84 μl; 85 μl; 86 μl; 87 μl; 88 μl; 89 μl; 90 μl; 91 μl; 92 μl; 93 μl; 94 μl; 95 μl; 96 μl; 97 μl; 98 μl; 99 μl; or 100 μl) of VEGF antagonist (e.g., a VEGF receptor fusion protein such as aflibercept) on a PRN basis.

In some embodiments, PRN treatment protocol calls for intervals between doctor visits to remain fixed (e.g., once every 2, 3, 4, 8, 12, 16 or 20 weeks) and decisions to carry out an injection of VEGF receptor fusion protein to be based on the anatomic findings at each respective visit. A capped PRN dosing regimen is PRN wherein subjects must be treated at a certain minimal frequency, e.g., at least once every 2 or 3 or 4 months.

Treat & Extend (T&E) regimens call for the time interval between doctor visits to be adjusted based on the patient's clinical course—e.g., if a subject shows no sign of an active disease (e.g., the macula remains dry, without any leakage), the next one or more intervals can be extended; if there is fluid accumulation, the next interval will be shortened. At each visit following T&E, an injection of VEGF receptor fusion protein will be performed; the current clinical status only has an impact on the duration of the next injection interval.

In an embodiment of the invention,
the method comprises administering the secondary doses to a subject who has received the initial dose
the method comprises administering the remaining secondary doses to a subject who has already received one or more secondary doses;
the method comprises administering one or more tertiary doses to a subject who has already received the secondary doses
the method comprises administering one or more tertiary doses to a subject who has already received one or more tertiary doses;
optionally wherein the subject receives doses, earlier in the regimen, in one pharmaceutical formulation and additional doses, later in the regimen, in a different pharmaceutical formulation, for example, comprising a different buffer (e.g., wherein one or more of the secondary doses are in one pharmaceutical formulation and the tertiary doses are in a different pharmaceutical formulation).

Dosing every “month” refers to dosing after about 28 days, about 4 weeks, or about 28±5 days and may encompass up to 5 weeks±5 days. Dosing every “4 weeks” or after “4 weeks” refers to dosing after about 28 days (±5 days), about a month or about 28 (±5 days), and may encompass up to every 5 weeks (±5 days). Dosing every “2-4 weeks” refers to dosing after about 2 weeks (±5 days), 3 weeks (±5 days) or 4 weeks (±5 days). Dosing every “8 weeks” or after “8 weeks” refers to dosing after about 2 months (±5 days) or about 56 (±5 days). Dosing every “12 weeks” or after “12 weeks” refers to dosing after about 3 months, about 84 days (±5 days), about 90 days (±5 days) or about 84 (±5 days). Dosing every “16 weeks” or after “16 weeks” refers to dosing after about 4 months or about 112 days (±5 days).

Dosing every “12-20 weeks” or after “12-20 weeks” refers to dosing after 12, 13, 14, 15, 16, 17, 18, 19 or 20 weeks (±5 days), preferably about 12-16 weeks (±5 days), about 12 weeks (±5 days), about 16 weeks (±5 days) or about 20 weeks (±5 days). Dosing every “12-20 weeks” refers to dosing after about 12, 13, 14, 15, 16, 17, 18, 19 or 20 weeks (±5 days), preferably about 12-16 weeks (±5 days), about 12 weeks (±5 days), about 16 weeks (±5 days) or about 20 weeks (±5 days).

A dose of greater than or equal to about 8 mg or > about 8 mg VEGF antagonist (e.g., aflibercept) includes 7.2 mg; 7.2-8.8 mg; 8.0 mg; 8.01 mg; 8.1 mg; 8.2 mg; 8.3 mg; 8.4 mg; 8.5 mg; 8.6 mg; 8.7 mg; 8.8 mg; 8.9 mg; 9 mg; 9.1 mg; 9.2 mg; 9.3 mg; 9.4 mg; 9.5 mg; 9.6 mg; 9.7 mg; 9.8 mg; 9.9 mg, 10.0 mg, 10.1 mg; 10.2 mg; 10.3 mg; 10.4 mg; 10.5 mg; 10.6 mg; 10.7 mg; 10.8 mg; 10.9 mg; 11 mg; 11.1 mg; 11.2 mg; 11.3 mg; 11.4 mg; 11.5 mg; 11.6 mg; 11.7 mg; 11.8 mg; 11.9 mg; 12 mg; 12.1 mg; 12.2 mg; 12.3 mg; 12.4 mg; 12.5 mg; 12.6 mg; 12.7 mg; 12.8 mg; 12.9 mg; 13 mg; 13.1 mg; 13.2 mg; 13.3 mg; 13.4 mg; 13.5 mg; 13.6 mg; 13.7 mg; 13.8 mg; 13.9 mg; 14 mg; 14.1 mg; 14.2 mg; 14.3 mg; 14.4 mg; 14.5 mg; 14.6 mg; 14.7 mg; 14.8 mg; 14.9 mg; 15 mg; 15.1 mg; 15.2 mg; 15.3 mg; 15.4 mg; 15.5 mg; 15.6 mg; 15.7 mg; 15.8 mg; 15.9 mg; 16 mg; 16.1 mg; 16.2 mg; 16.3 mg; 16.4 mg; 16.5 mg; 16.6 mg; 16.7 mg; 16.8 mg; 16.9 mg; 17 mg; 17.1 mg; 17.2 mg; 17.3 mg; 17.4 mg; 17.5 mg; 17.6 mg; 17.7 mg; 17.8 mg; 17.9 mg; 18 mg; 18.1 mg; 18.2 mg; 18.3 mg; 18.4 mg; 18.5 mg; 18.6 mg; 18.7 mg; 18.8 mg; 18.9 mg; 19 mg; 19.1 mg; 19.2 mg; 19.3 mg; 19.4 mg; 19.5 mg; 19.6 mg; 19.7 mg; 19.8 mg; 19.9 mg; or 20 mg (+ about 10%, + about 0.5, or + about 0.51 mg of any of the foregoing).

In an embodiment of the invention, a dosage of >8 mg VEGF antagonist is administered in a dose having a volume of about 100 μl or less, about 75 μl or less or about 70 μl or less, e.g., about 50 μl; 51 μl; 52 μl; 53 μl; 54 μl; 55 μl; 56 μl; 57 μl; 58 μl; 59 μl; 60 μl; 61 μl; 62 μl; 63 μl; 64 μl; 65 μl; 66 μl; 67 μl; 68 μl; 69 μl; 70 μl; 71 μl; 72 μl; 73 μl; 74 μl; 75 μl; 76 μl; 77 μl; 78 μl; 79 μl; 80 μl; 81 μl; 82 μl; 83 μl; 84 μl; 85 μl; 85-87 μl; 86 μl; 87 μl; 88 μl; 89 μl; 90 μl; 91 μl; 92 μl; 93 μl; 94 μl; 95 μl; 96 μl; 97 μl; 98 μl; 99 μl; or 100 μl (+ about 4, 4.45, 4.5, or 5 microliters).

The present invention includes methods wherein one or more additional, non-scheduled doses, in addition to any of the scheduled initial, secondary and/or tertiary doses of VEGF antagonist (e.g., a VEGF receptor fusion protein such as aflibercept) are administered to a subject. Such doses are typically administered at the discretion of the treating physician depending on the particular needs of the subject. The present invention also provides methods for treating angiogenic eye disorders (e.g., DR or DME) by administering to a subject in need thereof about ≥8 mg (for example, in about 100 μl or less, about 75 μl or less or about 70 μl or less, e.g., about 50 μl; 51 μl; 52 μl; 53 μl; 54 μl; 55 μl; 56 μl; 57 μl; 58 μl; 59 μl; 60 μl; 61 μl; 62 μl; 63 μl; 64 μl; 65 μl; 66 μl; 67 μl; 68 μl; 69 μl; 70 μl; 71 μl; 72 μl; 73 μl; 74 μl; 75 μl; 76 μl; 77 μl; 78 μl; 79 μl; 80 μl; 81 μl; 82 μl; 83 μl; 84 μl; 85 μl; 86 μl; 87 μl; 88 μl; 89 μl; 90 μl; 91 μl; 92 μl; 93 μl; 94 μl; 95 μl; 96 μl; 97 μl; 98 μl; 99 μl; or 100 μl) of VEGF antagonist (e.g., a VEGF receptor fusion protein such as aflibercept) on a PRN basis.

The present invention also provides methods for treating angiogenic eye disorders (preferably, DME or DR) by administering:
doses of about >8 mg (for example, in about 100 μl or less, about 75 μl or less or about 70 μl or less, e.g., about 50 μl; 51 μl; 52 μl; 53 μl; 54 μl; 55 μl; 56 μl; 57 μl; 58 μl; 59 μl; 60 μl; 61 μl; 62 μl; 63 μl; 64 μl; 65 μl; 66 μl; 67 μl; 68 μl; 69 μl; 70 μl; 71 μl; 72 μl; 73 μl; 74 μl; 75 μl; 76 μl; 77 μl; 78 μl; 79 μl; 80 μl; 81 μl; 82 μl; 83 μl; 84 μl; 85 μl; 86 μl; 87 μl; 88 μl; 89 μl; 90 μl; 91 μl; 92 μl; 93 μl; 94 μl; 95 μl; 96 μl; 97 μl; 98 μl; 99 μl; or 100 μl) about once every 4, 5, 6, 7, 8, 12, 16 or 20 weeks;
or
a single initial dose (e.g., about >8 mg, for example, in about 100 μl or less, about 75 μl or less or about 70 μl or less, e.g., about 50 μl; 51 μl; 52 μl; 53 μl; 54 μl; 55 μl; 56 μl; 57 μl; 58 μl; 59 μl; 60 μl; 61 μl; 62 μl; 63 μl; 64 μl; 65 μl; 66 μl; 67 μl; 68 μl; 69 μl; 70 μl; 71 μl; 72 μl; 73 μl; 74 μl; 75 μl; 76 μl; 77 μl; 78 μl; 79 μl; 80 μl; 81 μl; 82 μl; 83 μl; 84 μl; 85 μl; 86 μl; 87 μl; 88 μl; 89 μl; 90 μl; 91 μl; 92 μl; 93 μl; 94 μl; 95 μl; 96 μl; 97 μl; 98 μl; 99 μl; or 100 μl) of a VEGF antagonist (e.g., a VEGF receptor fusion protein such as aflibercept), followed by
one or more (e.g., 2, or 3 or 4 (preferably, 2 or 4)) secondary doses of the VEGF antagonist (e.g., a VEGF receptor fusion protein such as aflibercept), followed by
one or more tertiary doses of the VEGF antagonist (e.g., a VEGF receptor fusion protein such as aflibercept);
wherein each secondary dose is administered 2 to 4 (preferably, 4) weeks after the immediately preceding dose; and
wherein each tertiary dose is administered at about 4, 5, 6, 7 or 8 (e.g., 8) weeks after the immediately preceding dose; or
about >8 mg (for example, in about 100 μl or less, about 75 μl or less or about 70 μl or less, e.g., about 50 μl; 51 μl; 52 μl; 53 μl; 54 μl; 55 μl; 56 μl; 57 μl; 58 μl; 59 μl; 60 μl; 61 μl; 62 μl; 63 μl; 64 μl; 65 μl; 66 μl; 67 μl; 68 μl; 69 μl; 70 μl; 71 μl; 72 μl; 73 μl; 74 μl; 75 μl; 76 μl; 77 μl; 78 μl; 79 μl; 80 μl; 81 μl; 82 μl; 83 μl; 84 μl; 85 μl; 86 μl; 87 μl; 88 μl; 89 μl; 90 μl; 91 μl; 92 μl; 93 μl; 94 μl; 95 μl; 96 μl; 97 μl; 98 μl; 99 μl; or 100 μl) of VEGF antagonist (e.g., a VEGF receptor fusion protein such as aflibercept) once every about 4 weeks (q4w);
or
less than about 8 or 9 doses (e.g., about 5 doses or 6 doses) of about >8 mg (for example, in about 100 μl or less, about 75 μl or less or about 70 μl or less, e.g., about 50 μl; 51 μl; 52 μl; 53 μl; 54 μl; 55 μl; 56 μl; 57 μl; 58 μl; 59 μl; 60 μl; 61 μl; 62 μl; 63 μl; 64 μl; 65 μl; 66 μl; 67 μl; 68 μl; 69 μl; 70 μl; 71 μl; 72 μl; 73 μl; 74 μl; 75 μl; 76 μl; 77 μl; 78 μl; 79 μl; 80 μl; 81 μl; 82 μl; 83 μl; 84 μl; 85 μl; 86 μl; 87 μl; 88 μl; 89 μl; 90 μl; 91 μl; 92 μl; 93 μl; 94 μl; 95 μl; 96 μl; 97 μl; 98 μl; 99 μl; or 100 l) of a VEGF antagonist (e.g., a VEGF receptor fusion protein such as aflibercept) over the course of about 48 weeks.

Any dosing frequency specified herein may, in an embodiment of the invention, be expressed as the specific frequency “+5 days” (e.g., where “4 weeks” is stated, the present invention also includes embodiments such as 4 weeks+5 days).

“Sequentially administering” means that each dose of VEGF antagonist (e.g., aflibercept) is administered to the eye of a patient at a different point in time, e.g., on different days separated by a predetermined interval (e.g., hours, days, weeks or months). The present invention includes methods which comprise sequentially administering, to the eye of a patient, a single initial dose of a VEGF antagonist, followed by one or more secondary doses of the VEGF antagonist, followed by one or more tertiary doses of the VEGF antagonist.

An effective or therapeutically effective dose of VEGF antagonist (e.g., a VEGF receptor fusion protein such as aflibercept), for treating or preventing an angiogenic eye disorder refers to the amount of VEGF antagonist (e.g., a VEGF receptor fusion protein such as aflibercept) sufficient to alleviate one or more signs and/or symptoms of the disease or condition in the treated subject, whether by inducing the regression or elimination of such signs and/or symptoms or by inhibiting the progression of such signs and/or symptoms. In an embodiment of the invention, an effective or therapeutically effective dose of VEGF antagonist (e.g., a VEGF receptor fusion protein such as aflibercept) is about ≥8 mg every month, for 3 doses, followed by once every 12-20 weeks. In an embodiment of the invention, the alleviation of signs and/or symptoms is achievement, e.g., by 1 year, of a gain of ≥5, 10 or 15 letters BCVA (relative to baseline) (e.g., ≥5 letters improvement in a nAMD subject and/or 8-14 letters improvement in a DME patient/subject); achieving a BCVA ≥69 letters; achieving no fluid at foveal center; reduction in central retinal thickness (CRT) by about 150 micrometers or more (e.g., below 300 micrometers in an nAMD subject/patient; and/or reduction by at least about 200 micrometers in a DR or RVO patient/subject) or achievement of normal CRT (e.g., about 300 micrometers or less); and/or achievement of no leakage on fluorescein angiography. Baseline values refer to values prior to initiation of a treatment (pre-dose).

An “angiogenic eye disorder” means any disease of the eye which is caused by or associated with the growth or proliferation of blood vessels or by blood vessel leakage. Non-limiting examples of angiogenic eye disorders that are treatable or preventable using the methods of the present invention include:
age-related macular degeneration (neovascular (nAMD)),
macular edema (ME),
macular edema following retinal vein occlusion (ME-RVO),
retinal vein occlusion (RVO),
central retinal vein occlusion (CRVO),
branch retinal vein occlusion (BRVO),
diabetic macular edema (DME),
choroidal neovascularization (CNV),
iris neovascularization,
neovascular glaucoma,
post-surgical fibrosis in glaucoma,
proliferative vitreoretinopathy (PVR),
optic disc neovascularization,
corneal neovascularization,
retinal neovascularization,
vitreal neovascularization,
pannus,
pterygium,
vascular retinopathy,
diabetic retinopathies (DR) (e.g., non-proliferative diabetic retinopathy (e.g., characterized by a Diabetic Retinopathy Severity Scale (DRSS) level of about 47 or 53) or proliferative diabetic retinopathy; e.g., in a subject that does not suffer from DME), and
diabetic retinopathy in a patient who has diabetic macular edema (DME).

In an embodiment of the invention, a subject receiving a treatment for an angiogenic eye disorder as set forth herein (e.g., three monthly doses of about 8 mg aflibercept followed by doses of about 8 mg aflibercept every 12 weeks) achieves one or more of the following:
with respect to visual acuity (VA) or best corrected visual acuity (BCVA), achieving:
   No loss in visual acuity or BCVA or a gain in visual acuity or BCVA;
   no loss of visual acuity or BCVA, for example, by about week 4, 8, 9, 12, 16, 20, 24, 28, 32, 36, 40 or 44, following the initial dose e.g., according to ETDRS (Early Treatment Diabetic Retinopathy Study) chart or Snellen equivalent (e.g., no loss of 5, 10, or 15 or more ETDRS letters (e.g., no loss of 5 (or more), 6 (or more), 7 (or more), 8 (or more), 9 (or more), 10 (or more), 11 (or more), 12 (or more), 13 (or more), 14 (or more) or 15 (or more) letters) or Snellen equivalent),
   a gain in visual acuity or BCVA, for example, by about week 4, 8, 9, 12, 16, 20, 24, 28, 32, 36, 40 or 44 following the initial dose, e.g., according to ETDRS chart or Snellen equivalent (e.g., gaining 5 or more, 10 or more or 15 or more ETDRS letters (e.g., a gain of 5 (or more), 6 (or more), 7 (or more), 8 (or more), 9 (or more), 10 (or more), 11 (or more), 12 (or more), 13 (or more), 14 (or more) or 15 (or more) letters)); and/or
   a gain in BCVA of about 6 or 7 or 8 letters (or more) (e.g., according to ETDRS chart or Snellen equivalent) by week 8 and maintaining a gain of about 6 or 7 or 8 letters until at least about week 44;
with respect to central retinal thickness (CRT), achieving:
   a decrease in central retinal thickness;
   a decrease in central retinal thickness by at least about 80 micrometers;
   a decrease in central retinal thickness by at least about 123, 125, 131, 142, 147, 149, 150, 151, 156, 157, 158, 159, 161, 162, 166, 167, 168, 172, 173, 175, 177, 178 or 183 micrometers (or more), for example, by about week 4, 8, 9, 12, 16, 20, 24, 28, 32, 36, 40 or 44 following the initial dose, for example, wherein the baseline (prior to treatment) CRT, is about 488, 492, 497 or 516 micrometers;
   a decrease in CRT of about 47 micrometers (or more) from about week 12 to about week 20 following the initial dose, e.g., wherein the baseline CRT is about 516 micrometers;
   a decrease in CRT of about 17 micrometers (or more) from about week 24 to about week 32 following the initial dose, e.g., wherein the baseline CRT is about 516 micrometers;
   a decrease in CRT of about 18 micrometers (or more) from about week 36 to about week 44 following the initial dose, e.g., wherein the baseline CRT is about 516 micrometers;
   a decrease in central retinal thickness by at least about 4 or 24.3 micrometers (or more) from about week 12 to about week 16, for example, wherein the baseline CRT, prior to treatment, is about 516 micrometers;
   a decrease in CRT of about 123, 131 161 micrometers (or more) (e.g., by about week 4, 8, 12, 16 or 20) and maintaining the decrease until at least about week 44 following the initial dose; and/or
   a reduction in CRT of about 159, 160, 161 or 162 micrometers (or more) by about week 4 or 8 or 12 and maintaining a reduction of about 159, 160, 161 or 162 micrometers (or more) until at least about week 44;
with respect to retinal fluid, achieving:
   a dry retina (e.g., no IRF and no SRF; or no IRF; or no SRF, e.g., in the center subfield or in the macula, e.g., on SD-OCT);
   no fluid in the center subfield (e.g., no IRF and no SRF; or no IRF; or no SRF) by about week 4, 8, 9, 12, 16, 20, 24, 28, 32, 36, 40 or 44 following the initial dose (e.g., as measured by SD-OCT);
   no sub-retinal pigment epithelium (RPE) fluid; e.g., by about week 16, e.g., until at least about week 44 following the initial dose (e.g., as measured by SD-OCT);
   no SRF and IRF in the macula, e.g., by SD-OCT, e.g., by week 16 or week 44 following the initial dose, and/or
   maintenance of a dry retina, e.g., once achieved (e.g., at 16 weeks following the initial dose); until at least about week 44 (e.g., as measured by SD-OCT);
achieving:
   reduction in total lesion size by at least about 3.3 μm; and/or choroidal neovascularization (CNV) size of at least about 3.2 μm from baseline by about week 4, 8, 9, 12, 16, 20, 24, 28, 32, 36, 40 or 44, e.g., wherein baseline total lesion size is about 7.7 μm and/or baseline CNV size is about 7.5 μm;
   no significant increase in intraocular pressure from baseline by about week 4, 8, 9, 12, 16, 20, 24, 28, 32, 36, 40 or 44 or later (e.g., by no more than about 0.2 or 0.5 mmHg); and/or
   no significant increase in blood pressure (e.g., systolic (S) or diastolic (D)) from baseline by about week 4, 8, 9, 12, 16, 20, 24, 28, 32, 36, 40 or 44 or later (e.g., by no more than about 0.1 (S) or 0.9 (S), 1.0 (D) or 1.4 (D) mmHg); e.g., wherein baseline systolic pressure is about 125 mmHg or 129 mmHg and/or baseline diastolic pressure is about 72 mmHg or 74 mmHg;
   and/or
with respect to efficacy or safety:
   Efficacy and/or safety, in a subject suffering from DR or DME, similar to or greater than that of aflibercept which is intravitreally dosed at 2 mg approximately every 4 weeks for the first 5 injections followed by 2 mg approximately once every 8 weeks or once every 2 months, e.g., wherein efficacy is measured as an increase in visual acuity or BCVA and/or a reduction in central retinal thickness, achievement of dry retina (e.g., no IRF and/or SRF), e.g., wherein safety is as measured as the incidence of adverse events (treatment-emergent adverse events occurring anytime within 30 days of any injection) such as intraocular inflammation, clinically significant blood pressure increase, clinically significant intraocular pressure increase, visual impairment, vitreous floaters, vitreous detachment, iris neovascularization and/or vitreous hemorrhage; and/or
   Efficacy and/or safety, in a subject suffering from nAMD, similar to or greater than that of aflibercept which is intravitreally dosed at 2 mg approximately every 4 weeks for the first 3 injections followed by 2 mg approximately once every 8 weeks or once every 2 months, e.g., wherein efficacy is measured as 3 an increase in visual acuity or BCVA and/or a reduction in central retinal thickness, achievement of dry retina (e.g., no IRF and/or SRF), e.g., wherein safety is as measured as the incidence of adverse events (treatment-emergent adverse events occurring anytime within 30 days of any injection) such as intraocular inflammation, clinically significant blood pressure increase, clinically significant intraocular pressure increase, visual impairment, vitreous floaters, vitreous detachment, iris neovascularization and/or vitreous hemorrhage;
      for example, wherein such an effect (e.g., improvement of BCVA, CRT and/or retinal fluid) are achieved and/or maintained for as long as the subject is receiving the treatment regimen.

The center subfield of the retina is a 1 mm diameter area around the macula. The macula itself is about 6 mm in diameter.

The present invention also includes methods for achieving any one or more of the foregoing in a subject (e.g., increase in VA or BCVA, or decrease in CRT) suffering from an angiogenic eye disorder, e.g., nAMD, DR or DME, comprising administering to an eye of the subject, a single initial dose of about 8 mg or more of a VEGF antagonist (e.g., aflibercept), followed by one or more (e.g., 3, 4 or 5) secondary doses of about 8 mg or more of the VEGF antagonist, followed by one or more tertiary doses of about 8 mg or more of the VEGF antagonist; wherein each secondary dose is administered about 2 to 4 weeks after the immediately preceding dose; and wherein each tertiary dose is administered about 12 weeks after the immediately preceding dose.

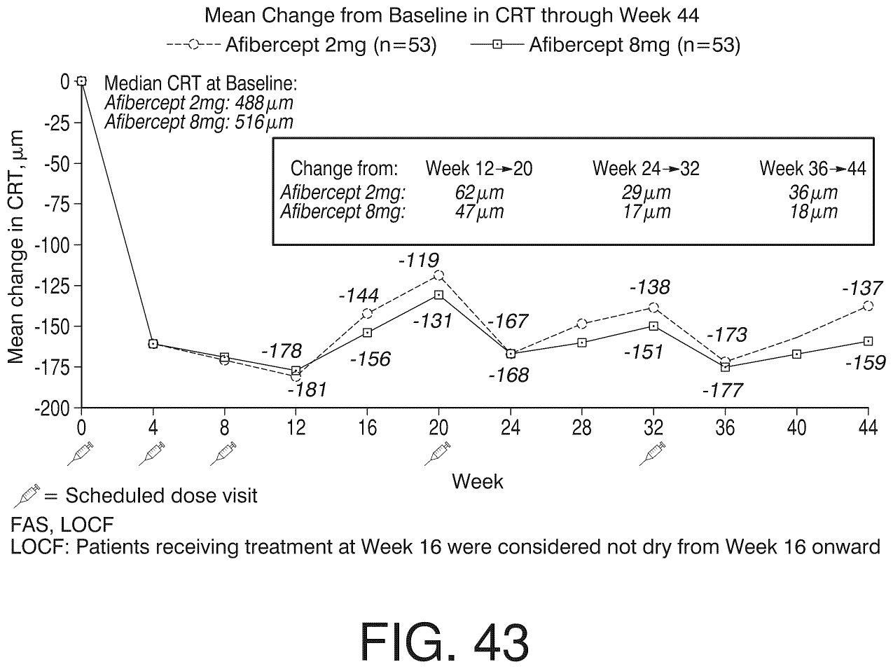

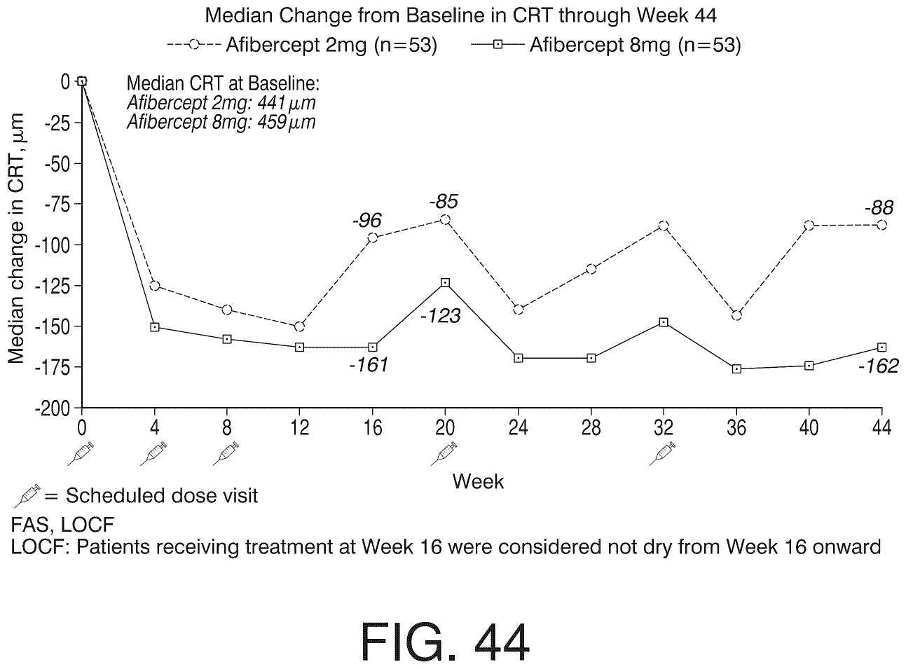

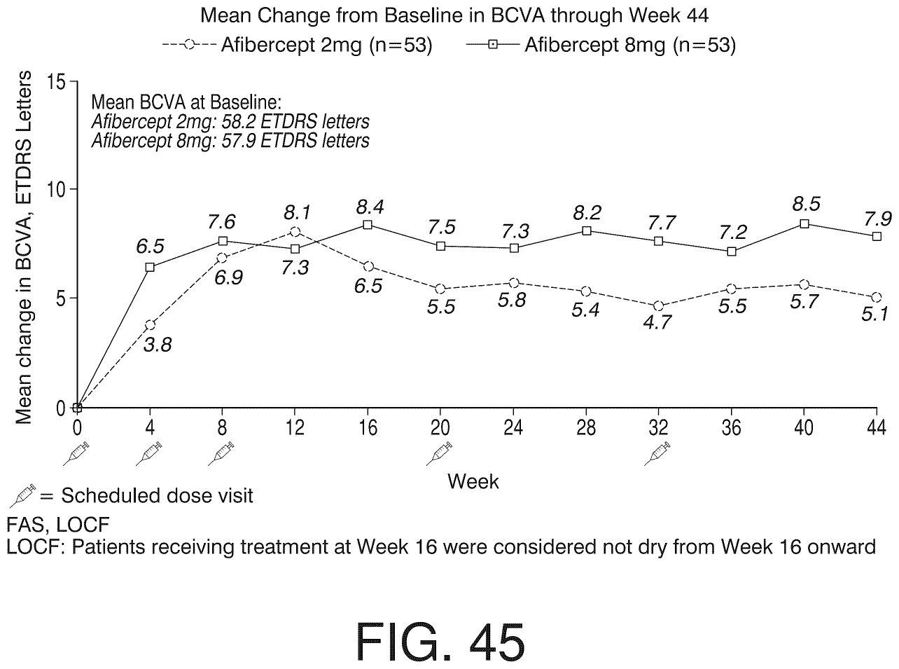

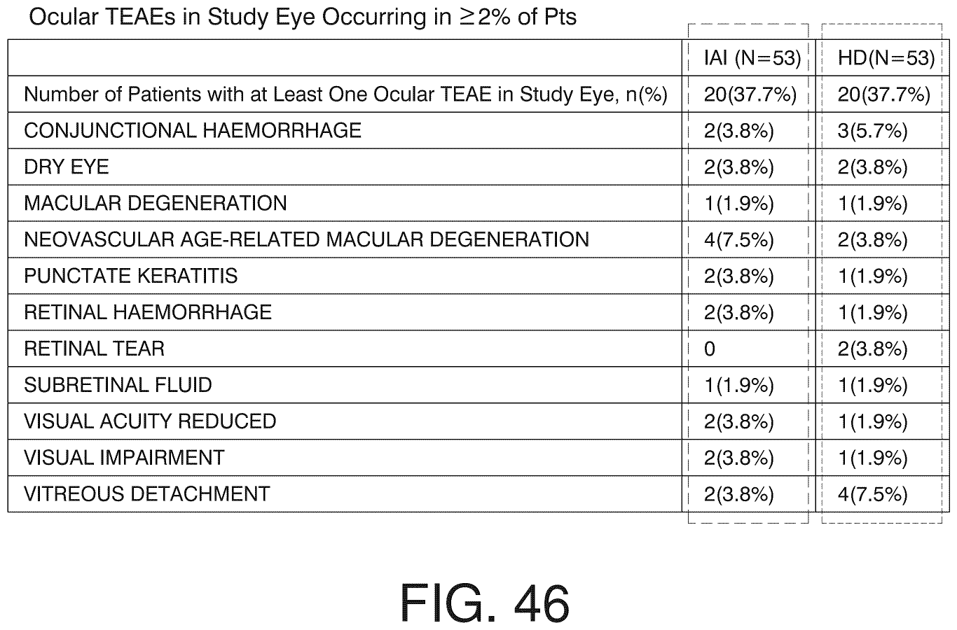

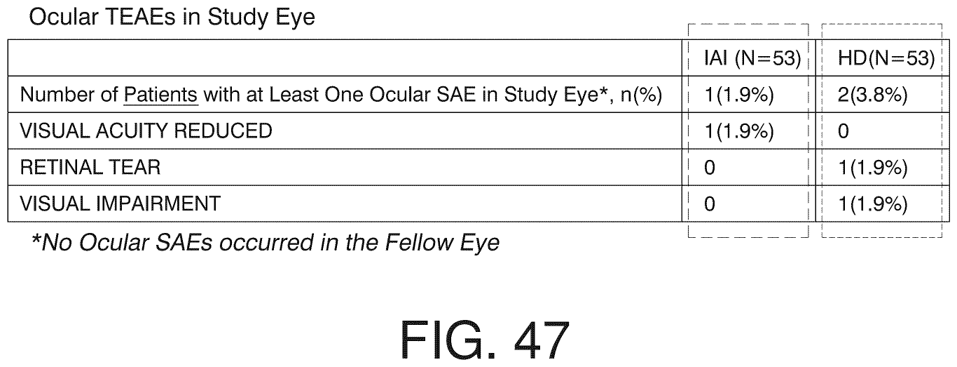

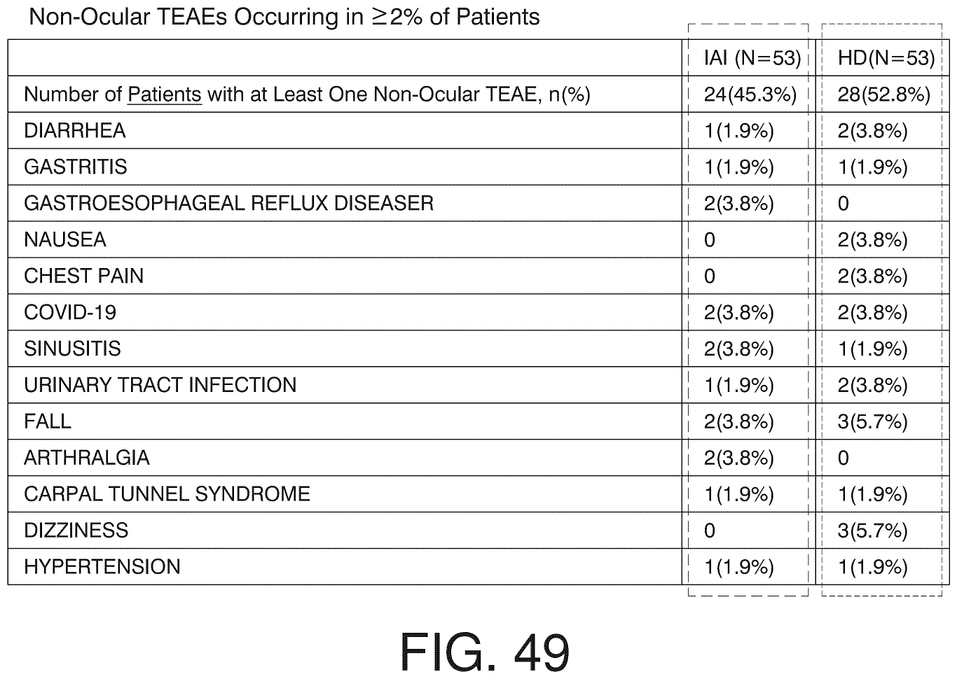

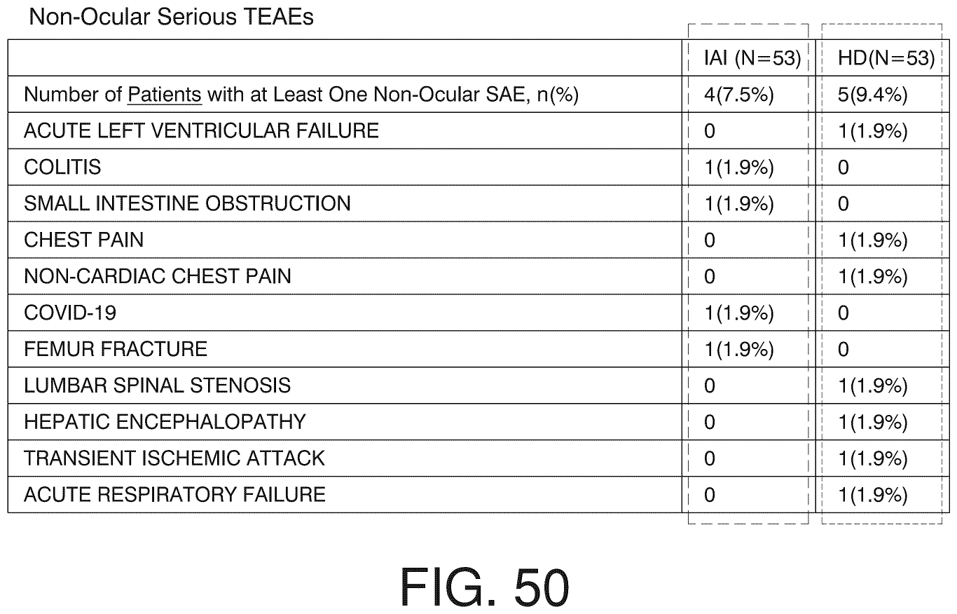

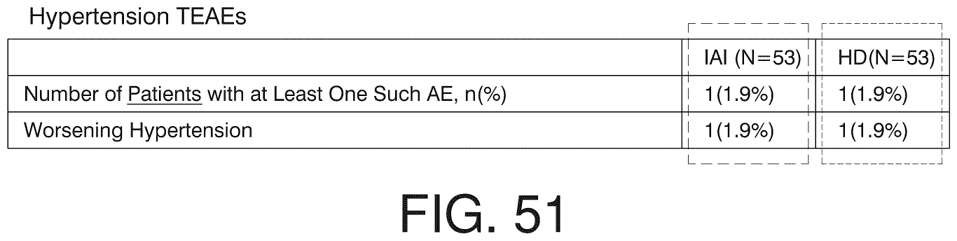

The present invention includes methods for treating or an angiogenic eye disorder (e.g., nAMD, DR, DME or ME-RVO), in a subject in need thereof, comprising administering to an eye of the subject, a single initial dose of about 8 mg or more of a VEGF antagonist (e.g., aflibercept), followed by one or more secondary doses of about 8 mg or more of the VEGF antagonist, followed by one or more tertiary doses of about 8 mg or more of the VEGF antagonist; wherein each secondary dose is administered about 2 to 4 weeks after the immediately preceding dose; and wherein each tertiary dose is administered about 12 weeks after the immediately preceding dose;
wherein the subject achieves a change in central retinal thickness, from baseline at the initiation of treatment, as depicted in FIG. 43 or FIG. 44 (or a greater reduction), e.g., by the timepoint shown for such a change; and/or
wherein the subject achieves a change in central retinal thickness, during the time period as set forth in the inset box of FIG. 43 (or a greater reduction), and/or
wherein the subject achieves a change in BCVA, from baseline at the initiation of treatment, as depicted in FIG. 45 (or a greater increase), e.g., by the timepoint shown for such a change (such change in BCVA can be in terms of ETDRS letters gained or in the Snellen equivalent); and/or
wherein the subject does not experience any one or more of the ocular TEAEs set forth in FIG. 46, ocular serious TEAEs set forth in FIG. 47, intraocular inflammation, any of the intraocular pressure events set forth in FIG. 48, non-ocular TEAEs set forth in FIG. 49, non-ocular serious TEAEs set forth in FIG. 50, hypertension TEAEs set forth in FIG. 51 and/or an APTC event.

The present invention includes embodiments wherein, at any point during a HDq12-20, HDq12, HDq16 or HDq20 treatment regimen, the patient can be switched to a PRN, capped PRN or T&E regimen. The PRN, capped PRN and/or T&E may be continued indefinitely or can be stopped at any point and then the HDq12-20, HDq12, HDq16 or HDq20 regimen is re-initiated at any phase thereof. Any HDq12-20, HDq12, HDq16 or HDq20 regimen can be preceded or followed by a period of PRN, capped PRN and/or T&E.

The present invention includes methods comprising administering the required doses of the HDq12 or HDq16 regimen, wherein each of the tertiary doses is administered 12 or 16 weeks after the immediately preceding dose, wherein the treatment interval between two tertiary doses is extended (e.g., from 12 weeks to 13, 14, 15, 16 or 20 weeks or from 16 weeks to 17, 18, 19, or 20 weeks), for example, until signs of disease activity or visual impairment deteriorate or recur and then either continuing dosing at the last tertiary interval used or the penultimate tertiary interval used.

The present invention includes methods comprising administering the required doses of the HDq12-20 or HDq12 or HDq16 or HDq20 regimen, wherein the treatment interval between any two tertiary doses is reduced (e.g., from 20 weeks to 19, 18, 17 or 16 weeks, from 16 weeks to 15, 14, 13, 12, 11, 10, 9, 8, 7, 6, 5, 4, 3, or 2 weeks or from 12 weeks to 11, 10, 9, 8, 7, 6, 5, 4, 3 or 2 weeks), for example, until signs of disease activity or visual impairment improve (e.g., BCVA stabilizes or improves and/or CRT stabilizes or reduces) whereupon, optionally, the interval between doses can be extended, e.g., back to a greater interval length.

For example, in an embodiment of the invention, the interval between doses, e.g., during the 12 week or 16 week dosing phase, can be lengthened, for example by 4 week intervals as appropriate (e.g., from 12 weeks to 16 or 16 weeks to 20 weeks), for example if:
<5 letter loss in BCVA, e.g., from week 12; and/or
CRT <300 μm on SD-OCT (or <320 μm on Spectralis SD-OCT).
   In an embodiment of the invention, the subject receives the initial, secondary and, then, 12 or 16 week tertiary intervals and, then, after about 1 year, extending the tertiary intervals to about 20 weeks.

In an embodiment of the invention, a method of treating an angiogenic eye disorder such as DR or DME as set forth herein includes the step of evaluating BCVA and/or CRT and lengthening the interval as discussed if one or both of the criteria are met.

For example, in an embodiment of the invention, the interval between doses, e.g., during the 12-20 week or 12 week or 16 week or 20 week dosing phase, can be shortened (e.g., from 12 or 16 or 20 weeks to 8 weeks; from 16 or 20 weeks to 12 weeks or from 20 weeks to 16 weeks), for example if:
greater than 5 or 10 letters are lost in BCVA (ETDRS or Snellen equivalent) (e.g., relative to the BCVA observed at about 12 or 16 weeks after treatment initiation) occurs, for example, due to or in association with persistent or worsening DR or DME; and/or greater than 25 or 50 micrometers increase in CRT is observed (e.g., relative to the CRT observed at about 12 weeks after treatment initiation).

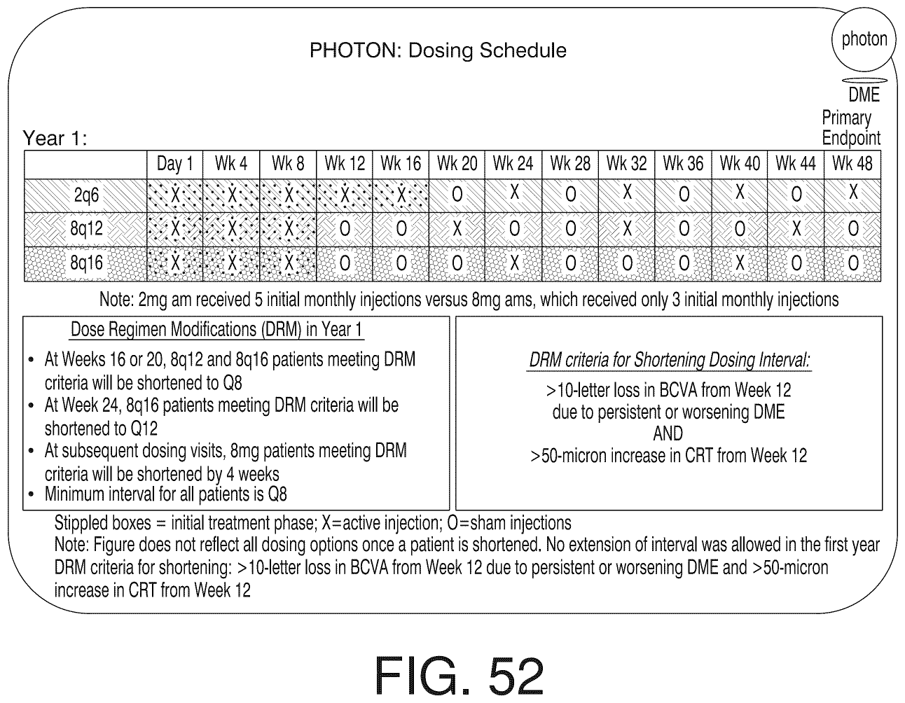

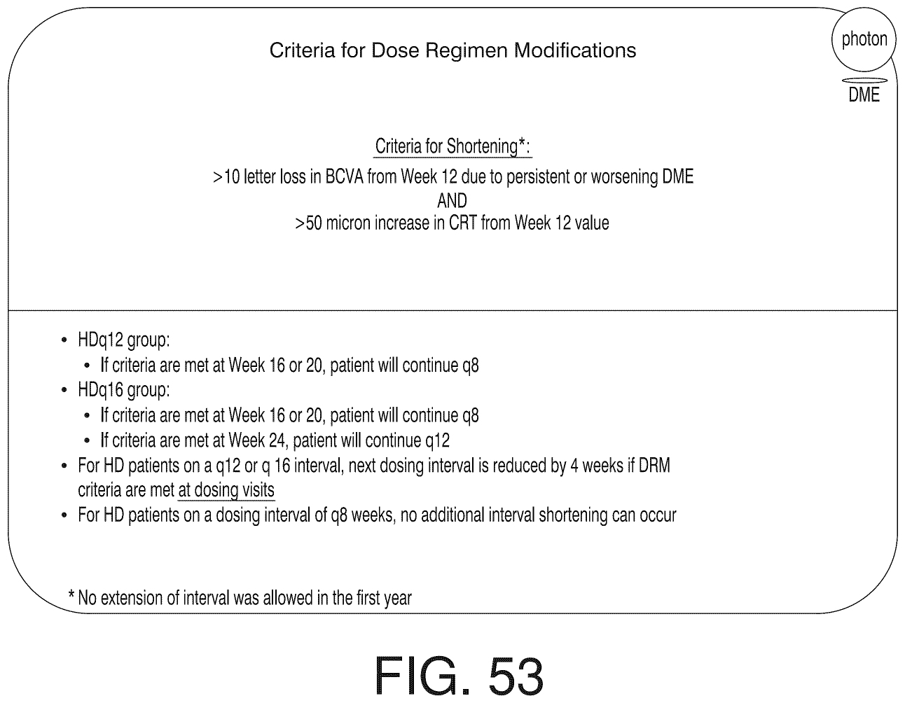

In an embodiment of the invention, if the criteria for reducing the interval between doses is met in a subject receiving the HDq12 regimen, e.g., at week 16 or 20, the interval between doses is decreased to 8 weeks. In an embodiment of the invention, if the criteria for reducing the interval between doses is met in a subject receiving the HDq16 regimen, e.g., at week 16 or 20, the interval between doses is decreased to 8 weeks; and if the criteria for reducing the interval between doses is met in a subject receiving the HDq16 regimen, e.g., at week 24, the interval between doses is decreased to 12 weeks. In an embodiment of the invention, the interval is not decreased to anything shorter than 8 weeks. In an embodiment of the invention, a method of treating an angiogenic eye disorder such as DR or DME as set forth herein includes the step of evaluating BCVA and/or CRT and shortening the interval as discussed if one or both of the criteria are met. See FIG. 52 and FIG. 53.

The present invention provides methods for treating angiogenic eye disorders (e.g., DR and/or DME) in a subject in need thereof, by sequentially administering initial loading doses (e.g., 2 mg or more, 4 mg or more or, preferably, about 8 mg or more of VEGF antagonist or inhibitor, for example, a VEGF receptor fusion protein such as aflibercept) (e.g., about every 2-4 or 3-5 weeks, preferably every 4 weeks; preferably, three initial loading doses) followed by additional doses every 12-20 weeks, preferably 12-16 weeks, 12 weeks, 16 weeks or 20 weeks wherein the subject achieves and/or maintains, e.g., by week 4, 8, 12, 16, 20, 24, 28, 32, 36, 40, 44, 48, 52, 56, 60, 64, 68, 72, 76, 80, 84, 88, 92 or 96 weeks after treatment initiation:
an improvement in Diabetic Retinopathy Severity Scale (DRSS), e.g., by at least 2 or 3 steps;
an improvement in best corrected visual acuity;
a dry retina;
a gain in best corrected visual acuity;
a gain in best corrected visual acuity of 1, 2, 3, 4, 5, 6, 7, 8, 9 or 10 letters or ≥5, ≥10 or ≥15 letters;
a BCVA of at least 69 letters;
a decrease in central retinal thickness (CRT), e.g., by about 100, 125, 150, 175 or 200 micrometers;
no vascular leakage as measured by fluorescein angiography (FA);
an improvement from pre-treatment baseline in National Eye Institute Visual Function Questionnaire (NEI-VFQ-25) total score;
a retina without fluid (total fluid, intraretinal fluid [IRF] and/or subretinal fluid [SRF]) at the foveal center and in center subfield;
maintenance of a fluid-free retina (total fluid, IRF and/or SRF at foveal center and in the center subfield);
a lack of macular edema;
a retina free of fluid on spectral domain optical coherence tomography (SD-OCT); and/or
Does not deviate from the HDq12 or HDq16 treatment regimen once started.

In an embodiment of the invention, a subject receiving a HDq12 or HDq16 or HDq20 treatment for an angiogenic eye disorder (e.g., DR and/or DME) as set forth herein achieves one or more of the following:
Does not receive a dose regimen modification, e.g., wherein the interval between doses (e.g., tertiary doses) is reduced from the HDq12-20 or HDq12 or HDq16 or HDq20 treatment regimen once started, e.g., for at least 4, 8, 12, 16, 20, 24, 28, 32, 36, 40, 44, 48, 52, 56, 60, 64, 68, 72, 76, 80, 84, 88, 92 or 96 weeks;
Receives 100% of all scheduled doses, e.g., for at least 4, 8, 12, 16, 20, 24, 28, 32, 36, 40, 44, 48, 52, 56, 60, 64, 68, 72, 76, 80, 84, 88, 92 or 96 weeks;
Non-inferior BVCA compared to that of aflibercept which is intravitreally dosed at 2 mg approximately every 4 weeks for the first 3, 4 or 5 injections followed by 2 mg approximately once every 8 weeks or once every 2 months;
Increase in BCVA (according to ETDRS letter score) of about 7, 8 or 9 letters by week 4, 8, 12, 16, 20, 24, 28, 32, 36, 40, 44, 48, 52, 56, 60, 64, 68, 72, 76, 80, 84, 88, 92 or 96, e.g., wherein the baseline BCVA is about 61, 62 and 63;
Improvement in BCVA, by 4 weeks after initiation of treatment, of about 4 or 5 letters (ETDRS or Snellen equivalent) when on HDq12 regimen; or of about 4 or 5 letters (ETDRS or Snellen equivalent) when on HDq16 regimen;
Improvement in BCVA, by 8 weeks after initiation of treatment, of about 6 letters (ETDRS or Snellen equivalent) when on HDq12 regimen; or of about 5 or 6 letters (ETDRS or Snellen equivalent) when on HDq16 regimen;
Improvement in BCVA, by 12 weeks after initiation of treatment, of about 6 or 7 letters (ETDRS or Snellen equivalent) when on HDq12 regimen; or of about 6 letters (ETDRS or Snellen equivalent) when on HDq16 regimen;
Improvement in BCVA, by 16 weeks after initiation of treatment, of about 6 or 7 letters (ETDRS or Snellen equivalent) when on HDq12 regimen; or of 7 letters (ETDRS or Snellen equivalent) when on HDq16 regimen;
Improvement in BCVA, by 20 weeks after initiation of treatment, of about 6 letters (ETDRS or Snellen equivalent) when on HDq12 regimen; or of about 6 letters (ETDRS or Snellen equivalent) when on HDq16 regimen;
Improvement in BCVA, by 24 weeks after initiation of treatment, of about 7 letters (ETDRS or Snellen equivalent) when on HDq12 regimen; or of about 5 or 6 letters (ETDRS or Snellen equivalent) when on HDq16 regimen;
Improvement in BCVA, by 28 weeks after initiation of treatment, of about 7 or 8 letters (ETDRS or Snellen equivalent) when on HDq12 regimen; or of about 7 or 8 letters (ETDRS or Snellen equivalent) when on HDq16 regimen;
Improvement in BCVA, by 32 weeks after initiation of treatment, of about 7 letters (ETDRS or Snellen equivalent) when on HDq12 regimen; or of about 7 or 8 letters (ETDRS or Snellen equivalent) when on HDq16 regimen;
Improvement in BCVA, by 36 weeks after initiation of treatment, of 8 letters (ETDRS or Snellen equivalent) when on HDq12 regimen; or of about 6 or 7 letters (ETDRS or Snellen equivalent) when on HDq16 regimen;
Improvement in BCVA, by 40 weeks after initiation of treatment, of about 8 letters (ETDRS or Snellen equivalent) when on HDq12 regimen; or of about 6 or 7 letters (ETDRS or Snellen equivalent) when on HDq16 regimen;
Improvement in BCVA, by 44 weeks after initiation of treatment, of about 8 letters (ETDRS or Snellen equivalent) when on HDq12 regimen; or of about 7 or 8 letters (ETDRS or Snellen equivalent) when on HDq16 regimen;
Improvement in BCVA, by 48 weeks after initiation of treatment, of about 8 or 9 letters (ETDRS or Snellen equivalent) when on HDq12 regimen; or of about 7 or 8 letters (ETDRS or Snellen equivalent) when on HDq16 regimen e.g., wherein the baseline BCVA is about 61, 62 or 63 letters (ETDRS or Snellen equivalent);
Improvement in BCVA, by 60 weeks after initiation of treatment, of about 8 or 9 letters (ETDRS or Snellen equivalent) when on HDq12 regimen; or of about 7 or 8 letters (ETDRS or Snellen equivalent) when on HDq16 regimen, e.g., wherein the baseline BCVA is about 61, 62 or 63 letters (ETDRS or Snellen equivalent);
An improvement in BCVA between weeks 48 and 60 of about 8 or 9 letters, or up to 40 letters (ETDRS or Snellen equivalent) when on the HDq12 regimen, e.g., when the basline BCVA is about 63 or 64; or an improvement in BCVA between weeks 48 and 60 of about 7 or 8 letters or up to 40 letters (ETDRS or Snellen equivalent) when on the HDq16 regimen e.g., when the basline BCVA is about 61 or 62;
An improvement in BCVA by about week 8 after initiation of treatment which is maintained (e.g., within about +1 or +2 ETDRS letters or Snellen equivalent) thereafter during the treatment regimen, e.g., to at least week 48;
Improvement in best corrected visual acuity (according to ETDRS letter score) (e.g., by week 12, 24, 36, 48, 60, 72, 84, 90 or 96 from start of treatment);
Improvement in best corrected visual acuity (BVCA) by week 4, week 8, week 12, week 16, week 20, week 24, week 28, week 32, week 36, week 40, week 44, or week 48 from start of treatment;
Increase in BCVA, e.g., as measured by the Early Treatment Diabetic Retinopathy Study (ETDRS) visual acuity chart or Snellen equivalent (e.g., by week 4, 8, 12, 16, 20, 24, 28, 32, 36, 40, 44 or 48 weeks from start of treatment) by ≥4 letters, ≥5 letters, ≥6 letters, ≥7 letters, ≥8 letters, >9 letters or >10 letters;
Between weeks 36 and 48, a change in BCVA score (according to ETDRS letter score) from initiation of treatment of about 7, 8 or 9, e.g., wherein the BCVA at any point between week 36 to 48 is about 60 or 70;
Between weeks 36 and 48, a change in BCVA score (according to ETDRS letter score) from initiation of treatment of up to 38 letters when on the HDq12 or HDq16 regimen, e.g., wherein BCVA at baseline is between about 27 and 79;
Between weeks 48 and 60, a change in BCVA score (according to ETDRS letter score) from initiation of treatment of about 7, 8 or 9; e.g., wherein the BCVA at any point between week 48 to 60 is about 69, 70, 71, 72 or 73;
A BCVA improvement, e.g., by week 48 following treatment initiation, of about 9 or 10 letters (ETDRS or Snellen equivalent) when baseline BCVA is about <73 ETDRS letters when on HDq12 regimen;
A BCVA improvement, e.g., by week 48 following treatment initiation, of about 5 or 6 letters (ETDRS or Snellen equivalent) when baseline BCVA is about >73 ETDRS letters when on HDq12 regimen;
A BCVA improvement, e.g., by week 48 following treatment initiation, of about 8 or 9 letters (ETDRS or Snellen equivalent) when baseline BCVA is about <73 ETDRS letters when on HDq16 regimen;
A BCVA improvement, e.g., by week 48 following treatment initiation, of about 4 or 5 letters (ETDRS or Snellen equivalent) when baseline BCVA is about >73 ETDRS letters when on HDq16 regimen;
A BCVA improvement, e.g., by week 48 following treatment initiation, of about 7 or 8 letters (ETDRS or Snellen equivalent) when baseline CRT is < about 400 micrometers when on HDq12 regimen;
A BCVA improvement, e.g., by week 48 following treatment initiation, of about 9 or 10 letters (ETDRS or Snellen equivalent) when baseline CRT is >400 micrometers when on HDq12 regimen;
A BCVA improvement, e.g., by week 48 following treatment initiation, of about 5 or 6 letters (ETDRS or Snellen equivalent) when baseline CRT is < about 400 micrometers when on HDq16 regimen;
A BCVA improvement, e.g., by week 48 following treatment initiation, of about 9 or 10 letters (ETDRS or Snellen equivalent) when baseline CRT is > about 400 micrometers when on HDq16 regimen;
Did not lose 5, 10 or 15 letters by week 4, 8, 12, 16, 20, 24, 28, 32, 36, 40, 44, 48, 52, 56, 60, 64, 68, 72, 76, 80, 84, 88, 92 or 96 (according to ETDRS letter score);
Gains at least 5, 10 or 15 letter by week 4, 8, 12, 16, 20, 24, 28, 32, 36, 40, 44, 48, 52, 56, 60, 64, 68, 72, 76, 80, 84, 88, 92 or 96 (according to ETDRS letter score);
Does not lose 5, 10, 15 or 69 letters or more BCVA (e.g., after week 4, 8, 12, 16, 20, 24, 28, 32, 36, 40, 44, 48, 52, 56, 60, 64, 68, 72, 76, 80, 84, 88, 92 or 96 from start of treatment);
Between weeks 48 and 60, a BCVA score (according to ETDRS letter score) of about 69, 70, 71, 72 or 73;
BCVA (according to ETDRS letter score) of at least about 69 letters, e.g., by week 48 or 60;
A BCVA by 4 weeks after initiation of treatment of about 68 letters (ETDRS or Snellen equivalent) when on the HDq12 regimen; or a BCVA of about 66 letters (ETDRS or Snellen equivalent) when on the HDq16 regimen;
A BCVA by 8 weeks after initiation of treatment of about 70 letters (ETDRS or Snellen equivalent) when on the HDq12 regimen; or a BCVA of about 67 letters (ETDRS or Snellen equivalent) when on the HDq16 regimen;
A BCVA by 12 weeks after initiation of treatment of about 70 letters (ETDRS or Snellen equivalent) when on the HDq12 regimen; or a BCVA of about 68 letters (ETDRS or Snellen equivalent) when on the HDq16 regimen;
A BCVA by 16 weeks after initiation of treatment of about 71 letters (ETDRS or Snellen equivalent) when on the HDq12 regimen; or a BCVA of about 69 letters (ETDRS or Snellen equivalent) when on the HDq16 regimen;
A BCVA by 20 weeks after initiation of treatment of about 70 letters (ETDRS or Snellen equivalent) when on the HDq12 regimen; or a BCVA of about 68 letters (ETDRS or Snellen equivalent) when on the HDq16 regimen;
A BCVA by 24 weeks after initiation of treatment of about 71 letters (ETDRS or Snellen equivalent) when on the HDq12 regimen; or a BCVA of about 67 letters (ETDRS or Snellen equivalent) when on the HDq16 regimen;
A BCVA by 28 weeks after initiation of treatment of about 72 letters (ETDRS or Snellen equivalent) when on the HDq12 regimen; or a BCVA of about 70 letters (ETDRS or Snellen equivalent) when on the HDq16 regimen;
A BCVA by 32 weeks after initiation of treatment of about 71 letters (ETDRS or Snellen equivalent) when on the HDq12 regimen; or a BCVA of about 70 letters (ETDRS or Snellen equivalent) when on the HDq16 regimen;
A BCVA by 36 weeks after initiation of treatment of about 71 letters (ETDRS or Snellen equivalent) when on the HDq12 regimen; or a BCVA of about 68 letters (ETDRS or Snellen equivalent) when on the HDq16 regimen;
A BCVA by 40 weeks after initiation of treatment of about 72 letters (ETDRS or Snellen equivalent) when on the HDq12 regimen; or a BCVA of about 69 letters (ETDRS or Snellen equivalent) when on the HDq16 regimen;
A BCVA by 44 weeks after initiation of treatment of about 72 letters (ETDRS or Snellen equivalent) when on the HDq12 regimen; or a BCVA of about 70 letters (ETDRS or Snellen equivalent) when on the HDq16 regimen;
A BCVA by 48 weeks after initiation of treatment of about 73 letters (ETDRS or Snellen equivalent) when on the HDq12 regimen; or a BCVA of about 70 letters (ETDRS or Snellen equivalent) when on the HDq16 regimen;
A BCVA between weeks 36 and 48 of about 71, 72, 73 or 74 (ETDRS or Snellen equivalent) when on the HDq12 regimen, e.g., when the basline BCVA is about 57, 58, 59, 60, 61, 62, 63 or 64; or a BCVA between weeks 36 and 48 of about 69, 70, 71, 72 or 73 (ETDRS or Snellen equivalent) when on the HDq16 regimen e.g., when the basline BCVA is about 55, 56, 57, 58, 59, 60, 61, or 62;
A BCVA between weeks 48 and 60 of about 69 or 70 or up to 94 (ETDRS or Snellen equivalent) when on the HDq12 regimen, e.g., when the basline BCVA is about 63 or 64; or a BCVA between weeks 48 and 60 of about 72 or 73 or up to 89 (ETDRS or Snellen equivalent) when on the HDq16 regimen e.g., when the basline BCVA is about 61 or 62;
Gain of >5, >10 or ≥15 letters BCVA (according to ETDRS letter score) (e.g., by week 4, 8, 12, 16, 20, 24, 28, 32, 36, 40, 44, 48, 52, 56, 60, 64, 68, 72, 76, 80, 84, 88, 92 or 96 from start of treatment);
≥2 step improvement in Diabetic Retinopathy Severity Scale (DRSS) (e.g., by week 4, 8, 12, 16, 20, 24, 28, 32, 36, 40, 44, 48, 52, 56, 60, 72, 84, 90 or 96 from start of treatment);
≥3 step improvement in diabetic retinopathy severity scale (DRSS) (e.g., by week 4, 8, 12, 16, 20, 24, 28, 32, 36, 40, 44, 48, 52, 56, 60, 72, 84, 90 or 96 weeks from start of treatment);
Retina without fluid (total fluid, intraretinal fluid [IRF] and/or subretinal fluid [SRF]) at the foveal center or center subfield (e.g., by week 4, 8, 12, 16, 20, 24, 28, 32, 36, 40, 44, 48, 52, 56, 60, 64, 68, 72, 76, 80, 84, 88, 92 or 96 from start of treatment) (e.g., as measured by optical coherence tomography (OCT);
No vascular leakage in the retina as measured by fluorescein angiography (FA) (e.g., by week 4, 8, 12, 16, 20, 24, 28, 32, 36, 40, 44, 48, 52, 56, 60, 64, 68, 72, 76, 80, 84, 88, 92 or 96 from start of treatment);
Maintenance of a fluid-free retina (total fluid, IRF and/or SRF at foveal center and in the center subfield) (e.g., by week 4, 8, 12, 16, 20, 24, 28, 32, 36, 40, 44, 48, 52, 56, 60, 64, 68, 72, 76, 80, 84, 88, 92 or 96 from start of treatment);
Reduction in total area of fluorescein leakage within ETDRS grid (mm2) at week 48 or 60 by about 12, 12.6, 13, 13.6, 13.9 or 14 mm2 or more, or up to about 57 or 68 mm2 (e.g., as measured by fluorescein angiography);
Reduction in total area of fluorescein leakage within ETDRS grid (mm2) at week 48 by about 13. 13.3, 13.9 or 14 mm2 or more (e.g., up to about 52 mm2) (e.g., as measured by fluorescein angiography) when on the HDq12 regimen;
Reduction in total area of fluorescein leakage within ETDRS grid (mm2) at week 48 by about 7, 7.7, 8, 9, 9.4 or 10 mm2 or more (e.g., up to about 55 mm2) (e.g., as measured by fluorescein angiography) when on the HDq16 regimen;
Retina free of fluid on spectral domain optical coherence tomography (SD-OCT) (e.g., by week 12, 24, 36, 48, 60, 72, 84, 90 or 96 from start of treatment);
Retina without fluid (total fluid, intraretinal fluid [IRF] and/or subretinal fluid [SRF]) at the foveal center (e.g., by week 4, 8, 12, 16, 20, 24, 28, 32, 36, 40, 44 or 48 weeks from start of treatment);
Dry retina (e.g., by week 4, 8, 12, 16, 20, 24, 28, 32, 36, 40, 44, 48, 52, 56, 60, 64, 68, 72, 76, 80, 84, 88, 92 or 96 from start of treatment);
Foveal center without fluid (e.g., by week 4, 8, 12, 16, 20, 24, 28, 32, 36, 40, 44, 48, 52, 56, 60, 64, 68, 72, 76, 80, 84, 88, 92 or 96 from start of treatment) (e.g., as measured by optical coherence tomography (OCT);
A change in central retinal thickness, by 4 weeks after initiation of treatment of about −118 or −118.3 micrometers (+17, 18 or 19 micrometers) when on the HDq12 regimen; or of about −124 or −125 or −124.9 or −125.5 micrometers (+17, 18 or 19 micrometers) when on the HDq16 regimen;
A change in central retinal thickness, by 8 weeks after initiation of treatment of about −137 or −137.4 micrometers (+17, 18 or 19 micrometers) when on the HDq12 regimen; or of about −139 or −140 or −139.6 or −140.3 micrometers (+17, 18 or 19 micrometers) when on the HDq16 regimen;
A change in central retinal thickness, by 12 weeks after initiation of treatment of about −150 or −150.1 micrometers (+17, 18 or 19 micrometers) when on the HDq12 regimen; or of about −152 or −153 or −152.7 or −153.4 micrometers (+17, 18 or 19 micrometers) when on the HDq16 regimen;
A change in central retinal thickness, by 16 weeks after initiation of treatment of about −139 or −139.4 micrometers (+17, 18 or 19 micrometers) when on the HDq12 regimen; or of about −145 or −146 or −145.5 or −146.4 micrometers (+17, 18 or 19 micrometers) when on the HDq16 regimen;
A change in central retinal thickness, by 20 weeks after initiation of treatment of about −117 or −117.1 micrometers (+17, 18 or 19 micrometers) when on the HDq12 regimen; or of about −112 or −113 or −112.5 or −113.3 micrometers (+17, 18 or 19 micrometers) when on the HDq16 regimen;
A change in central retinal thickness, by 24 weeks after initiation of treatment of about −158 or −158.1 micrometers (+17, 18 or 19 micrometers) when on the HDq12 regimen; or of about −103 or −104 or −103.8 or −104.3 micrometers (+17, 18 or 19 micrometers) when on the HDq16 regimen;
A change in central retinal thickness, by 28 weeks after initiation of treatment of about −146 or −147 or −146.7 micrometers (+17, 18 or 19 micrometers) when on the HDq12 regimen; or of about −162 or −162.3 micrometers (+17, 18 or 19 micrometers) when on the HDq16 regimen;
A change in central retinal thickness, by 32 weeks after initiation of treatment of about −132 micrometers (+17, 18 or 19 micrometers) when on the HDq12 regimen; or of about −145 or −146 or −145.8 micrometers (+17, 18 or 19 micrometers) when on the HDq16 regimen;
A change in central retinal thickness, by 36 weeks after initiation of treatment of about −168 or −168.1 micrometers (+17, 18 or 19 micrometers) when on the HDq12 regimen; or of about −124 or −125 or −124.7 or −125.2 micrometers (+17, 18 or 19 micrometers) when on the HDq16 regimen;
A change in central retinal thickness, by 40 weeks after initiation of treatment of about −163 micrometers (+17, 18 or 19 micrometers) when on the HDq12 regimen; or of about −122 or −123 or −122.5 or −123.1 micrometers (+17, 18 or 19 micrometers) when on the HDq16 regimen;
A change in central retinal thickness, by 44 weeks after initiation of treatment of about −147 or −148 or −147.4 micrometers (+17, 18 or 19 micrometers) when on the HDq12 regimen; or of about −164 or −164.1 or −164.3 micrometers (+17, 18 or 19 micrometers) when on the HDq16 regimen;
A change in central retinal thickness, by 48 weeks after initiation of treatment of about −171 or −172 or −171.7, −172, −173, −174, −175, −176 or −176.77 micrometers (+5, 6, 7, 8, 9, 10, 11, 12, 13, 14, 15, 16, 17, 18 or 19 micrometers) when on the HDq12 regimen; or of about −148 or −149 or −148.3 or −149.4 micrometers (+9, 10, 11, 12, 13, 14, 15, 16, 17, 18 or 19 micrometers) when on the HDq16 regimen, e.g., wherein baseline CRT is about 449, 450, 455 or 460 micrometers;
A change in central retinal thickness, by 60 weeks after initiation of treatment of about −181, −182, −181.95, −176, −176.24 or −177 (+6, 10, 17, 18 or 19 micrometers) micrometers when on the HDq12 regimen (e.g., wherein the baseline CRT is about 460 micrometers); or of about −166, −166.26, −167 or −167.18 micrometers (+8, 9, 10, 17, 18 or 19 micrometers) when on the HDq16 regimen (e.g., wherein the baseline CRT is about 457 micrometers);
A change in central retinal thickness of about −118 or −119 or −118.3 micrometers, between initiation of treatment (week 0) and week 4 when on the HDq12 regimen;
A change in central retinal thickness of about −19, −20 or −19.1 micrometers, between weeks 4 and 8 when on the HDq12 regimen;
A change in central retinal thickness of about −12, −13 or −12.7 micrometers, between weeks 8 and 12 when on the HDq12 regimen;
A change in central retinal thickness of about −40, or −41 micrometers, between weeks 20 and 24 when on the HDq12 regimen;
A change in central retinal thickness of about −36, −37 or −36.1 micrometers, between weeks 32 and 36 when on the HDq12 regimen;
A change in central retinal thickness of about −24, −25 or −24.3 micrometers, between weeks 44 and 48 when on the HDq12 regimen;
A change in central retinal thickness of about −124, −125 or −124.9 micrometers, between initiation of treatment (week 0) and week 4 when on the HDq16 regimen;
A change in central retinal thickness of about −14, −15 or −14.7 micrometers, between weeks 4 and 8 when on the HDq16 regimen;
A change in central retinal thickness of about −13, −14 or −13.1 micrometers, between weeks 8 and 12 when on the HDq16 regimen;
A change in central retinal thickness of about −58, −59 or −58.5 micrometers, between weeks 24 and 28 when on the HDq16 regimen;
A change in central retinal thickness of about −41, −42 or −41.6 micrometers, between weeks 40 and 44 when on the HDq16 regimen;
A reduction in CRT by week 4, 5, 6, 7 or 8 after initiation of treatment which is maintained (e.g., within about +17, +18 or +19 micrometers) thereafter during the treatment regimen, e.g., to at least week 48;
Decrease in central retinal thickness (CRT), for example, by about 100, 125, 150, 175 or 200 micrometers (e.g., by week 12, 24, 36, 48, 60, 72, 84, 90 or 96 from start of treatment);
Reduction in CRT of about 148-182 micrometers (e.g., 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183) by week 48 or 60 (e.g., as measured by optical coherence tomography (OCT)), for example, wherein the baseline CRT is about 449, 450, 455 or 460 micrometers;
Decrease in central retinal thickness (CRT), for example, by at least about 100, 125, 130, 135, 140, 145, 149, 150, 155, 160, 165, 170, 171, 172, 173, 174 or 175 micrometers (e.g., by week 4, 8, 12, 16, 20, 24, 28, 32, 36, 40, 44 or 48 from start of treatment);
Ocular (e.g., intraocular pressure) and non-ocular safety (e.g., hypertensive events or APTC events) or death rate, in a subject suffering from an angiogenic eye disorder, e.g., DR or DME, similar to that of aflibercept which is intravitreally dosed at 2 mg approximately every 4 weeks for the first 3, 4 or 5 injections followed by 2 mg approximately once every 8 weeks or once every 2 months;
At about 0.1667 days after the first dose, free aflibercept concentration in plasma of about 0.149 (+0.249) mg/l; e.g., wherein at baseline free aflibercept concentration in plasma not detectable, for example, wherein the subject has not received intravitreal VEGF inhibitor (e.g., aflibercept) treatment for at least 12 weeks;
At about 0.3333 days after the first dose, free aflibercept concentration in plasma of about 0.205 (+0.250) mg/l; e.g., wherein at baseline free aflibercept concentration in plasma not detectable, for example, wherein the subject has not received intravitreal VEGF inhibitor (e.g., aflibercept) treatment for at least 12 weeks;
At about 1 day after the first dose, free aflibercept concentration in plasma of about 0.266 (+0.211) mg/l; e.g., wherein at baseline free aflibercept concentration in plasma not detectable, for example, wherein the subject has not received intravitreal VEGF inhibitor (e.g., aflibercept) treatment for at least 12 weeks;
At about 2 days after the first dose, free aflibercept concentration in plasma of about 0.218 (+0.145) mg/l; e.g., wherein at baseline free aflibercept concentration in plasma not detectable, for example, wherein the subject has not received intravitreal VEGF inhibitor (e.g., aflibercept) treatment for at least 12 weeks;
At about 4 days after the first dose, free aflibercept concentration in plasma of about 0.140 (+0.0741) mg/l; e.g., wherein at baseline free aflibercept concentration in plasma not detectable, for example, wherein the subject has not received intravitreal VEGF inhibitor (e.g., aflibercept) treatment for at least 12 weeks;
At about 7 days after the first dose, free aflibercept concentration in plasma of about 0.0767 (+0.0436) mg/l; e.g., wherein at baseline free aflibercept concentration in plasma not detectable, for example, wherein the subject has not received intravitreal VEGF inhibitor (e.g., aflibercept) treatment for at least 12 weeks;
At about 14 days after the first dose, free aflibercept concentration in plasma of about 0.0309 (+0.0241) mg/l; e.g., wherein at baseline free aflibercept concentration in plasma not detectable, for example, wherein the subject has not received intravitreal VEGF inhibitor (e.g., aflibercept) treatment for at least 12 weeks;
At about 21 days after the first dose, free aflibercept concentration in plasma of about 0.0171 (+0.0171) mg/l; e.g., wherein at baseline free aflibercept concentration in plasma not detectable, for example, wherein the subject has not received intravitreal VEGF inhibitor (e.g., aflibercept) treatment for at least 12 weeks;
At about 28 days after the first dose, free aflibercept concentration in plasma of about 0.00730 (+0.0113) mg/l; e.g., wherein at baseline free aflibercept concentration in plasma not detectable, for example, wherein the subject has not received intravitreal VEGF inhibitor (e.g., aflibercept) treatment for at least 12 weeks;
At about 0.1667 days after the first dose, adjusted bound aflibercept concentration in plasma of about 0.00698 (+0.0276) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
At about 0.3333 days after the first dose, adjusted bound aflibercept concentration in plasma of about 0.00731 (+0.0279) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
At about 1 days after the first dose, adjusted bound aflibercept concentration in plasma of about 0.0678 (+0.0486) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
At about 2 days after the first dose, adjusted bound aflibercept concentration in plasma of about 0.138 (+0.0618) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
At about 4 days after the first dose, adjusted bound aflibercept concentration in plasma of about 0.259 (+0.126) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
At about 7 days after the first dose, adjusted bound aflibercept concentration in plasma of about 0.346 (+0.151) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
At about 14 days after the first dose, adjusted bound aflibercept concentration in plasma of about 0.374 (+0.110) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
At about 21 days after the first dose, adjusted bound aflibercept concentration in plasma of about 0.343 (+0.128) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
At about 28 days after the first dose, adjusted bound aflibercept concentration in plasma of about 0.269 (+0.149) mg/l; e.g., wherein at baseline there is about 0.00583 mg/l (+0.0280) adjusted bound aflibercept concentration, for example, wherein the subject has not received intravitreal VEGF inhibitor (e.g., aflibercept) treatment for at least 12 weeks;
The maximum concentration of free aflibercept in the plasma is reached about 0.965 (e.g., about 1) day after the first dose;
Reaches a maximum concentration of about 0.310 mg/l (+0.263) free aflibercept in the plasma;
Individual free aflibercept concentration (Cmax) in the plasma of from about 0 to about 1.08 mg/L (0.1, 0.2, 0.3, 0.4, 0.5, 0.6, 0.7, 0.8, 0.9, 1.0 or 1.1 mg/l);
Free aflibercept in the plasma maximum (mg/l) per dose (mg) of aflibercept of about 0.0388 (+0.00328) mg/l/mg;
The maximum concentration of adjusted bound aflibercept in the plasma is reached about 14 day after the first dose;
Reaches a maximum concentration of about 0.387 mg/l (+0.135) adjusted bound aflibercept in the plasma;
Adjusted bound aflibercept concentration in the plasma of from about 0.137 to about 0.774 mg/L (0.1, 0.2, 0.3, 0.4, 0.5, 0.6, 0.7, 0.8 mg/l);
Adjusted bound aflibercept concentration in the plasma maximum (mg/l) per dose (mg) of aflibercept of about 0.0483 (+0.0168) mg/l/mg;
Does not have anti-drug antibodies against aflibercept after 48 or 60 weeks of treatment;
Improvement from pre-treatment baseline in National Eye Institute Visual Function Questionnaire (NEI-VFQ-25) total score (e.g., by week 12, 24, 36, 48, 60, 72, 84, 90 or 96 from start of treatment), e.g., by about 4, 5 or 6 when on the HDq12 regimen or by about 2, 3 or 4 when on the HDq16 regimen; e.g., wherein the baseline score is about 76 or 77;
Lack of macular edema (e.g., by week 12, 24, 36, 48, 60, 72, 84, 90 or 96 from start of treatment); and/or
Efficacy and/or safety, in a subject suffering from DR or DME, similar to that of aflibercept which is intravitreally dosed at 2 mg approximately every 4 weeks for the first 5 injections followed by 2 mg approximately once every 8 weeks or once every 2 months, e.g., wherein efficacy is measured as increase in BCVA and/or reduction in central retinal thickness, e.g., wherein safety is as measured as the incidence of adverse events (treatment-emergent adverse events occurring anytime within 30 days of any injection) such as blood pressure increase, intraocular pressure increase, visual impairment, vitreous floaters, vitreous detachment, iris neovascularization and/or vitreous hemorrhage.

Thus, the present invention provides the following:
A method for achieving a non-inferior BVCA compared to that of aflibercept which is intravitreally dosed at 2 mg approximately every 4 weeks for the first 3, 4 or 5 injections followed by 2 mg approximately once every 8 weeks or once every 2 months; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving an increase in BCVA (according to ETDRS letter score) of about 7, 8 or 9 letters by week 60 wherein the baseline BCVA is about 61, 62 and 63; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a BCVA (according to ETDRS letter score) of at least about 69 letters by week 48 or 60; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a BCVA wherein there is not a loss of 5, 10, 15 or 69 letters or more after week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving an improvement in best corrected visual acuity (according to ETDRS letter score) by week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving an improvement in best corrected visual acuity (BVCA) by week 4, week 8, week 12, week 16, week 20, week 24, week 28, week 32, week 36, week 40, week 44, week 48 or week 60 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving between weeks 48 and 60 from treatment initiation, a BCVA score (according to ETDRS letter score) of about 69, 70, 71, 72 or 73; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving between weeks 36 and 48, a change in BCVA score (according to ETDRS letter score) from initiation of treatment of about 7, 8 or 9, wherein the BCVA at any point between week 36 to 48 is about 60 or 70; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving between weeks 48 and 60, a change in BCVA score (according to ETDRS letter score) from initiation of treatment of about 7, 8 or 9 wherein the BCVA at any point between week 48 to 60 is about 69, 70, 71, 72 or 73; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving an increase in BCVA as measured by the Early Treatment Diabetic Retinopathy Study (ETDRS) visual acuity chart or Snellen equivalent by week 4, 8, 12, 16, 20, 24, 28, 32, 36, 40, 44, 48 or 60 weeks from start of treatment by ≥4 letters, ≥5 letters, ≥6 letters, ≥7 letters, ≥8 letters, ≥9 letters or ≥10 letters; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a BCVA wherein there is not a loss of 5, 10 or 15 letters by week 48 or 60 (according to ETDRS letter score); in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving an gain at least 5, 10 or 15 letter by week 48 or 60 (according to ETDRS letter score); in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving an improvement in BCVA, by 4 weeks after initiation of treatment, of about 4 or 5 letters (ETDRS or Snellen equivalent) when on a HDq12 regimen; or of about 4 or 5 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 8 weeks after initiation of treatment, of about 6 letters (ETDRS or Snellen equivalent) when on a HDq12 regimen; or of about 5 or 6 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 12 weeks after initiation of treatment, of about 6 or 7 letters (ETDRS or Snellen equivalent) when on aHDq12 regimen; or of about 6 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 16 weeks after initiation of treatment, of about 6 or 7 letters (ETDRS or Snellen equivalent) when on a HDq12 regimen; or of 7 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 20 weeks after initiation of treatment, of about 6 letters (ETDRS or Snellen equivalent) when on a HDq12 regimen; or of about 6 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 24 weeks after initiation of treatment, of about 7 letters (ETDRS or Snellen equivalent) when on a HDq12 regimen; or of about 5 or 6 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 28 weeks after initiation of treatment, of about 7 or 8 letters (ETDRS or Snellen equivalent) when on a HDq12 regimen; or of about 7 or 8 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 32 weeks after initiation of treatment, of about 7 letters (ETDRS or Snellen equivalent) when on a HDq12 regimen; or of about 7 or 8 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 36 weeks after initiation of treatment, of 8 letters (ETDRS or Snellen equivalent) when on a HDq12 regimen; or of about 6 or 7 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 40 weeks after initiation of treatment, of about 8 letters (ETDRS or Snellen equivalent) when on a HDq12 regimen; or of about 6 or 7 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 44 weeks after initiation of treatment, of about 8 letters (ETDRS or Snellen equivalent) when on a HDq12 regimen; or of about 7 or 8 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA, by 48 weeks after initiation of treatment, of about 8 or 9 letters (ETDRS or Snellen equivalent) when on a HDq12 regimen; or of about 7 or 8 letters (ETDRS or Snellen equivalent) when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving an improvement in BCVA by about week 8 after initiation of treatment which is maintained (within about ±1 or ±2 ETDRS letters or Snellen equivalent) thereafter during the treatment regimen to at least week 48 or 60; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a BCVA by 4 weeks after initiation of treatment of about 68 letters (ETDRS or Snellen equivalent) when on the a HDq12 regimen; or a BCVA of about 66 letters (ETDRS or Snellen equivalent) when on the a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 8 weeks after initiation of treatment of about 70 letters (ETDRS or Snellen equivalent) when on the a HDq12 regimen; or a BCVA of about 67 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 12 weeks after initiation of treatment of about 70 letters (ETDRS or Snellen equivalent) when on the HDq12 regimen; or a BCVA of about 68 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 16 weeks after initiation of treatment of about 71 letters (ETDRS or Snellen equivalent) when on the HDq12 regimen; or a BCVA of about 69 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 20 weeks after initiation of treatment of about 70 letters (ETDRS or Snellen equivalent) when on the HDq12 regimen; or a BCVA of about 68 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 24 weeks after initiation of treatment of about 71 letters (ETDRS or Snellen equivalent) when on the HDq12 regimen; or a BCVA of about 67 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 28 weeks after initiation of treatment of about 72 letters (ETDRS or Snellen equivalent) when on the HDq12 regimen; or a BCVA of about 70 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 32 weeks after initiation of treatment of about 71 letters (ETDRS or Snellen equivalent) when on the HDq12 regimen; or a BCVA of about 70 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 36 weeks after initiation of treatment of about 71 letters (ETDRS or Snellen equivalent) when on the HDq12 regimen; or a BCVA of about 68 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 40 weeks after initiation of treatment of about 72 letters (ETDRS or Snellen equivalent) when on the HDq12 regimen; or a BCVA of about 69 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 44 weeks after initiation of treatment of about 72 letters (ETDRS or Snellen equivalent) when on the HDq12 regimen; or a BCVA of about 70 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA by 48 weeks after initiation of treatment of about 73 letters (ETDRS or Snellen equivalent) when on the HDq12 regimen; or a BCVA of about 70 letters (ETDRS or Snellen equivalent) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a BCVA improvement, by week 48 following treatment initiation, of about 9 or 10 letters (ETDRS or Snellen equivalent) when baseline BCVA is about <73 ETDRS letters when on HDq12 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen after the immediately preceding dose.
A method for achieving a BCVA improvement by week 48 following treatment initiation, of about 5 or 6 letters (ETDRS or Snellen equivalent) when baseline BCVA is about ≥73 ETDRS letters when on HDq12 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) after the immediately preceding dose.
A method for achieving a BCVA improvement, by week 48 following treatment initiation, of about 8 or 9 letters (ETDRS or Snellen equivalent) when baseline BCVA is about ≤73 ETDRS letters when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 16 weeks (HDq16 regimen) after the immediately preceding dose.
A method for achieving a BCVA improvement, by week 48 following treatment initiation, of about 4 or 5 letters (ETDRS or Snellen equivalent) when baseline BCVA is about >73 ETDRS letters when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 16 weeks (HDq16 regimen) after the immediately preceding dose.
A method for achieving a BCVA improvement, by week 48 following treatment initiation, of about 7 or 8 letters (ETDRS or Snellen equivalent) when baseline CRT is < about 400 micrometers when on a HDq12 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) after the immediately preceding dose.
A method for achieving a BCVA improvement, by week 48 following treatment initiation, of about 9 or 10 letters (ETDRS or Snellen equivalent) when baseline CRT is ≥400 micrometers when on a HDq12 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) after the immediately preceding dose.
A method for achieving a BCVA improvement, by week 48 following treatment initiation, of about 5 or 6 letters (ETDRS or Snellen equivalent) when baseline CRT is < about 400 micrometers when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 16 weeks (HDq16 regimen) after the immediately preceding dose.
A method for achieving a BCVA improvement, by week 48 following treatment initiation, of about 9 or 10 letters (ETDRS or Snellen equivalent) when baseline CRT is ≥ about 400 micrometers when on a HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 16 weeks (HDq16 regimen) after the immediately preceding dose.
A method for achieving a gain of ≥5, ≥10 or ≥15 letters BCVA (according to ETDRS letter score) by week 12, 24, 36, 48, 60, 72, 84, 90 or 96 from start of treatment); in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) or 20 weeks (HDq20 regimen) after the immediately preceding dose.
A method for achieving a ≥2 or ≥3 step improvement in Diabetic Retinopathy Severity Scale (DRSS) by week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a ≥2 step improvement in diabetic retinopathy severity scale (DRSS) by 4, 8, 12, 16, 20, 24, 28, 32, 36, 40, 44, 48 or 60 weeks from start of treatment); in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a retina without fluid (total fluid, intraretinal fluid [IRF] and/or subretinal fluid [SRF]) at the foveal center and in center subfield by week 12, 24, 36, 48, 60, 72, 84, 90 or 96 from start of treatment as measured by optical coherence tomography (OCT); in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a no vascular leakage from the retina as measured by fluorescein angiography (FA) by week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a maintenance of a fluid-free retina (total fluid, IRF and/or SRF at foveal center and in the center subfield) by week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a reduction in total area of fluorescein leakage within ETDRS grid (mm2) at week 48 or 60 by about 12, 13 or 14 mm2 or more as measured by fluorescein angiography; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a retina free of fluid on spectral domain optical coherence tomography (SD-OCT) by week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a retina without fluid (total fluid, intraretinal fluid [IRF] and/or subretinal fluid [SRF]) at the foveal center by week 4, 8, 12, 16, 20, 24, 28, 32, 36, 40, 44 or 48 weeks from start of treatment); in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a dry retina by week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a foveal center without fluid by week 12, 24, 36, 48, 60, 72, 84, 90 or 96 from start of treatment as measured by optical coherence tomography (OCT); in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a change in central retinal thickness, by 4 weeks after initiation of treatment of about −118 or −118.3 micrometers (±17, 18 or 19 micrometers) when on the HDq12 regimen; or of about −124 or −125 or −124.9 or −125.5 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 8 weeks after initiation of treatment of about −137 or −137.4 micrometers (±17, 18 or 19 micrometers) when on the HDq12 regimen; or of about −139 or −140 or −139.6 or −140.3 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 12 weeks after initiation of treatment of about −150 or −150.1 micrometers (±17, 18 or 19 micrometers) when on the HDq12 regimen; or of about −152 or −153 or −152.7 or −153.4 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 16 weeks after initiation of treatment of about −139 or −139.4 micrometers (±17, 18 or 19 micrometers) when on the HDq12 regimen; or of about −145 or −146 or −145.5 or −146.4 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 20 weeks after initiation of treatment of about −117 or −117.1 micrometers (±17, 18 or 19 micrometers) when on the HDq12 regimen; or of about −112 or −113 or −112.5 or −113.3 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 24 weeks after initiation of treatment of about −158 or −158.1 micrometers (±17, 18 or 19 micrometers) when on the HDq12 regimen; or of about −103 or −104 or −103.8 or −104.3 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 28 weeks after initiation of treatment of about −146 or −147 or −146.7 micrometers (±17, 18 or 19 micrometers) when on the HDq12 regimen; or of about −162 or −162.3 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 32 weeks after initiation of treatment of about −132 micrometers (±17, 18 or 19 micrometers) when on the HDq12 regimen; or of about −145 or −146 or −145.8 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 36 weeks after initiation of treatment of about −168 or −168.1 micrometers (±17, 18 or 19 micrometers) when on the HDq12 regimen; or of about −124 or −125 or −124.7 or −125.2 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 40 weeks after initiation of treatment of about −163 micrometers (±17, 18 or 19 micrometers) when on the HDq12 regimen; or of about −122 or −123 or −122.5 or −123.1 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 44 weeks after initiation of treatment of about −147 or −148 or −147.4 micrometers (±17, 18 or 19 micrometers) when on the HDq12 regimen; or of about −164 or −164.1 or −164.3 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 48 weeks after initiation of treatment of about −171 or −172 or −171.7 micrometers (±17, 18 or 19 micrometers) when on the HDq12 regimen; or of about −148 or −149 or −148.3 or −149.4 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a change in central retinal thickness, by 60 weeks after initiation of treatment of about −181.95 or −176.24 (±17, 18 or 19 micrometers) micrometers when on the HDq12 regimen; or of about −166.26 or −167.18 micrometers (±17, 18 or 19 micrometers) when on the HDq16 regimen; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.
A method for achieving a reduction in CRT by week 4, 5, 6, 7 or 8 after initiation of treatment which is maintained (within about ±17, ±18 or ±19 micrometers) thereafter during the treatment regimen to at least week 48 or 60; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a decrease in central retinal thickness (CRT) by about 100, 125, 150, 175 or 200 micrometers by week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a reduction in CRT of about 148-182 micrometers by week 48 or 60 as measured by optical coherence tomography (OCT) wherein the baseline CRT is about 449, 450, 455 or 460 micrometers; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a decrease in central retinal thickness (CRT) by at least about 100, 125, 130, 135, 140, 145, 149, 150, 155, 160, 165, 170, 171, 172, 173, 174 or 175 micrometers by week 4, 8, 12, 16, 20, 24, 28, 32, 36, 40, 44, 48 or 60 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 0.1667 days after the first dose, free aflibercept in plasma of about 0.149 (±0.249) mg/l; wherein at baseline free aflibercept in plasma not detectable, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 0.3333 days after the first dose, free aflibercept in plasma of about 0.205 (±0.250) mg/l; wherein at baseline free aflibercept in plasma not detectable, for example, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 1 day after the first dose, free aflibercept in plasma of about 0.266 (±0.211) mg/l; wherein at baseline free aflibercept in plasma not detectable, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 2 days after the first dose, free aflibercept in plasma of about 0.218 (±0.145) mg/l; wherein at baseline free aflibercept in plasma not detectable, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 4 days after the first dose, free aflibercept in plasma of about 0.140 (±0.0741) mg/l; wherein at baseline free aflibercept in plasma not detectable, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 7 days after the first dose, free aflibercept in plasma of about 0.0767 (±0.0436) mg/l; wherein at baseline free aflibercept in plasma not detectable, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 14 days after the first dose, free aflibercept in plasma of about 0.0309 (±0.0241) mg/l; wherein at baseline free aflibercept in plasma not detectable, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 21 days after the first dose, free aflibercept in plasma of about 0.0171 (±0.0171) mg/l; wherein at baseline free aflibercept in plasma not detectable, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 28 days after the first dose, free aflibercept in plasma of about 0.00730 (±0.0113) mg/l; wherein at baseline free aflibercept in plasma not detectable, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 0.1667 days after the first dose, adjusted bound aflibercept in plasma of about 0.00698 (±0.0276) mg/l; wherein at baseline there is about 0.00583 mg/l (±0.0280) adjusted bound aflibercept, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 0.3333 days after the first dose, adjusted bound aflibercept in plasma of about 0.00731 (±0.0279) mg/l; wherein at baseline there is about 0.00583 mg/l (±0.0280) adjusted bound aflibercept, for example, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 1 days after the first dose, adjusted bound aflibercept in plasma of about 0.0678 (±0.0486) mg/l; wherein at baseline there is about 0.00583 mg/l (±0.0280) adjusted bound aflibercept, for example, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 2 days after the first dose, adjusted bound aflibercept in plasma of about 0.138 (±0.0618) mg/l; wherein at baseline there is about 0.00583 mg/l (±0.0280) adjusted bound aflibercept, wherein the subject has not received intravitreal aflibercept protein treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 4 days after the first dose, adjusted bound aflibercept in plasma of about 0.259 (±0.126) mg/l; wherein at baseline there is about 0.00583 mg/l (±0.0280) adjusted bound aflibercept, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 7 days after the first dose, adjusted bound aflibercept in plasma of about 0.346 (±0.151) mg/l; wherein at baseline there is about 0.00583 mg/l (±0.0280) adjusted bound aflibercept, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 14 days after the first dose, adjusted bound aflibercept in plasma of about 0.374 (±0.110) mg/l; wherein at baseline there is about 0.00583 mg/l (±0.0280) adjusted bound aflibercept, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 21 days after the first dose, adjusted bound aflibercept in plasma of about 0.343 (±0.128) mg/l; wherein at baseline there is about 0.00583 mg/l (±0.0280) adjusted bound aflibercept, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving at about 28 days after the first dose, adjusted bound aflibercept in plasma of about 0.269 (±0.149) mg/l; wherein at baseline there is about 0.00583 mg/l (±0.0280) adjusted bound aflibercept, wherein the subject has not received intravitreal aflibercept treatment for at least 12 weeks; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a maximum level of free aflibercept in the plasma that is reached about 0.965 day after the first dose; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a maximum level of about 0.310 mg/l (±0.263) free aflibercept in the plasma; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving free aflibercept in the plasma of from about 0 to about 1.08 mg/L (0.1, 0.2, 0.3, 0.4, 0.5, 0.6, 0.7, 0.8, 0.9, 1.0 or 1.1 mg/l); in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving free aflibercept in the plasma maximum (mg/l) per dose (mg) of aflibercept of about 0.388 (±0.0328) mg/l/mg; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a maximum level of adjusted bound aflibercept in the plasma that is reached about 14 days after the first dose; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a maximum level of about 0.387 mg/l (±0.135) adjusted bound aflibercept in the plasma; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving adjusted bound aflibercept in the plasma of from about 0.137 to about 0.774 mg/L; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving adjusted bound aflibercept in the plasma maximum (mg/l) per dose (mg) of aflibercept of about 0.483 (±0.0168) mg/l/mg; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving undetectable plasma concentrations of anti-drug antibodies against aflibercept after 48 or 60 weeks of treatment; in a subject in need thereof having an angiogenic eye disorder (preferably DR and/or DME) comprising administering to an eye of the subject, a single initial dose of about 8 mg or more of aflibercept, followed by one or more secondary doses of about 8 mg or more of the aflibercept, followed by one or more tertiary doses of about 8 mg or more of the aflibercept; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving an improvement from pre-treatment baseline in National Eye Institute Visual Function Questionnaire (NEI-VFQ) total score by week 12, 24, 36, 48, 60, 72, 84, 90 or 96 from start of treatment; in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving a lack of macular edema by week 12, 24, 36, 48, 60, 72, 84, 90 or 96 from start of treatment in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12-20 weeks (HDq12-20 regimen) or 12 weeks (HDq12 regimen) or 16 weeks (HDq16 regimen) or 20 weeks (HDq20 regimen) after the immediately preceding dose.
A method for achieving:
   a change in central retinal thickness of about −118 or -119 or −118.3 micrometers, between initiation of treatment (week 0) and week 4 when on the HDq12 regimen;
   a change in central retinal thickness of about −19, −20 or −19.1 micrometers, between weeks 4 and 8 when on the HDq12 regimen;
   a change in central retinal thickness of about −12, −13 or −12.7 micrometers, between weeks 8 and 12 when on the HDq12 regimen;
   a change in central retinal thickness of about −40, or −41 micrometers, between weeks 20 and 24 when on the HDq12 regimen;
   a change in central retinal thickness of about −36, −37 or −36.1 micrometers, between weeks 32 and 36 when on the HDq12 regimen;
   a change in central retinal thickness of about −24, −25 or −24.3 micrometers, between weeks 44 and 48 when on the HDq12 regimen;
   a change in central retinal thickness of −4, −5 or −4.5 micrometers, between weeks 48 and 60 when on the HDq12 regimen;
   a change in central retinal thickness of about −124, −125 or −124.9 micrometers, between initiation of treatment (week 0) and week 4 when on the HDq16 regimen;
   a change in central retinal thickness of about −14, −15 or −14.7 micrometers, between weeks 4 and 8 when on the HDq16 regimen;
   a change in central retinal thickness of about −13, −14 or −13.1 micrometers, between weeks 8 and 12 when on the HDq16 regimen;
   a change in central retinal thickness of about −58, −59 or −58.5 micrometers, between weeks 24 and 28 when on the HDq16 regimen;
   a change in central retinal thickness of about −41, −42 or −41.6 micrometers, between weeks 40 and 44 when on the HDq16 regimen; and/or
   a change in central retinal thickness of −18, −19 or −18.9 micrometers, between weeks 48 and 60 when on the HDq16 regimen,
   in a subject in need thereof having an angiogenic eye disorder (preferably DR and/or DME) comprising administering to an eye of the subject, a single initial dose of about 8 mg or more of a VEGF receptor fusion protein, followed by one or more secondary doses of about 8 mg or more of the VEGF receptor fusion protein, followed by one or more tertiary doses of about 8 mg or more of the VEGF receptor fusion protein; wherein each secondary dose is administered about 2 to 4 weeks after the immediately preceding dose; and wherein each tertiary dose is administered about 12 weeks (HDq12 regimen) or 16 weeks (HDq16 regimen) after the immediately preceding dose.

Referring now to FIGS. 54-57, an example of a Randomized, Single-Masked, Active-Controlled Phase 2 Study of the Safety, Tolerability, and Efficacy of Repeated Doses of High-Dose Aflibercept in Patients with Neovascular Age-Related Macular Degeneration (CANDELA trial) is shown and described herein.

This phase 2, multi-center, randomized, single-masked study in patients with nAMD investigated the efficacy, safety, and tolerability of HD (8 mg doses) versus IAI (2 mg doses). The Study dosing regimen is summarized in FIG. 54. The Study consisted of a screening/baseline period, a treatment period, and an end of study (EOS) visit at week 44. Patients were seen monthly through week 44. One hundred and six eligible patients were randomized into 2 groups in a 1:1 ratio. One group received IAI and the other received HD. The investigational product was administered intravitreally (IVT) monthly for 3 initial injections (baseline, week 4, and week 8), followed by additional doses at weeks 20 and 32. At weeks 24, 28, 36 and 40, patients were evaluated and given a dose (at their randomized dose level) if either of the following criteria are met (PRN criteria): Loss of ≥5 letters from week 20 BCVA due to disease progression; or Anatomical findings that are considered vision threatening, such as worsening or persistent retinal fluid, new or worsening PED (pigment epithelial detachment), new or persistent hemorrhage, etc. The following are the portions of the protocol by which the CANDELA human clinical trial was conducted.

The study also includes a pharmacokinetic (PK) sub-study, with dense blood sampling (dense PK sub-study) for systemic drug concentrations and PK assessments for approximately 15 patients from each group from selected sites. Additional patients (up to approximately 50% more in each treatment group) may be enrolled in the dense PK sub-study to ensure adequate data is are captured. The dosing schedule for the IAI and HD groups are set forth in FIG. 54. Results at week 16 (n=55)-values as reported at the time for patients reaching week 16. After 16 weeks of study duration, patients receiving the 8 mg doses of aflibercept (HD dosing regimen) maintained (on average) greater vision improvements and anatomical improvements than that of patients receiving the 2 mg dose (IAI dosing regimen) (relative to baseline).

Patients (Completers) receiving the HD dosing regimen maintained a greater mean change in best corrected visual acuity (7.4) than the IAI patients (5.2) at week 16. Anatomical improvements were also remarkable at week 16. The central retinal thickness (CRT) of patients receiving the HD dosing regimen remained below that of patients (Completers) receiving the IAI dosing regimen—a mean change of −142 micrometers in HD patients vs −133 micrometers in IAI patients at week 16. Measures of retinal dryness were also strikingly better in patients receiving the HD dosing regimen. Overall, more patients (Completers) receiving the HD regimen had complete resolution of intraretinal/subretinal fluid at week 16. The proportion of HD patients with dry retinas at week 16 was 44% whereas the proportion of IAI patients with dry retinas was just 9%. A dry retina was regarded as a retina exhibiting no intraretinal fluid (IRF) and subretinal fluid (SRF). Conversely, a greater proportion of patients receiving the IAI dosing regimen had “not-dry” retinas at week 16 than that of HD patients (86% vs 56%, respectively). Fewer HD patients exhibited IRF (IRF only or both IRF and SRF) or SRF (SRF only or both IRF and SRF) at week 16 than IAI patients. Similar trends were apparent when the retinal fluid status (Dry, IRF only, SRF only or both IRF and SRF) of IAI and HD patients at baseline, week 4, week 8, week 12 and week 16 was observed. The number of treatments in the HD and IAI treatment groups (“All patients”) was comparable.

The HD and IAI dosing regimens were generally well tolerated. The ocular treatment-emergent adverse events (TEAEs), intraocular pressure (IOP), non-ocular TEAEs, non-ocular SAEs, hypertension AEs, mean systolic blood pressure, mean diastolic blood pressure, mean IOP, mean change of IOP were comparable in each treatment group.

Results—additional patients reaching week 16 (n=106)—values as reported at the time for a larger set of patients reaching week 16. At week 16, a higher proportion of these patients in the HD aflibercept 8 mg group had no retinal or subretinal fluid (50.9%, n=27/53) in the center (1 mm) subfield on optical coherence tomography compared to patients treated with IAI EYLEA 2 mg (34.0%, n=18/53) (p=0.08). During the initial 16 weeks of the trial, treatment emergent adverse events (TEAEs) in the study eye occurred in 17.0% (9 of 53) of aflibercept 8 mg patients and 22.6% (12 of 53) of EYLEA 2 mg patients. AEs that occurred more frequently in the aflibercept 8 mg group were conjunctival hemorrhage (5.7% aflibercept 8 mg, 3.8% EYLEA 2 mg) and vitreous detachment (3.8% aflibercept 8 mg, 1.9% EYLEA 2 mg). Serious ocular AEs (SAEs) occurred in two patients overall, one in the aflibercept 8 mg group (retinal tear) and one in the EYLEA 2 mg group (visual acuity reduced). No intraocular inflammation, occlusive vasculitis, arterial thromboembolic events (adjudicated according to the Anti-Platelet Trialists' Collaboration definitions) or death in either patient group were identified through week 16.

The proportion of HD patients achieving a dry retina (no IRF and no SRF in the central subfield on SD-OCT (spectral domain optical coherence tomography)) was 51% whereas the proportion of IAI patients was 34% (last observation carried forward (LOCF) values). HD patients without IRF (dry or with SRF only) was 70% (IAI patients: 68%) and without SRF (dry or with IRF only) was 70% (IAI patients: 51%) (LOCF). At week 16, there was a greater median and mean decrease in central retinal thickness from baseline in HD patients (−161.0 micrometers, −156.2 micrometers, respectively) relative to IAI patients (−96.0 micrometers, −143.5 micrometers, respectively) (LOCF). The mean change from baseline to week 12 in choroidal neovascularization and total lesion size is set forth in showing greater reductions in the HD group relative to the IAI group. Regarding best corrected visual acuity (BCVA), HD patients achieved a mean change, from baseline through week 16, of 8.4 (ETDRS letters) whereas IAI patients achieved 6.5 (LOCF). A smaller proportion of HD patients lost letters than IAI patients; and a greater proportion of HD patients gained letters than IAI patients at week 16.

The proportion of HD patients receiving additional treatment at week 16 (19%) was smaller than that of IAI patients (27%). Also, the occurrence of ocular TEAEs through week 16 was 17% among HD patients and 22.6% among IAI patients. The occurrence of ocular serious treatment emergent adverse events and intraocular inflammation TEAEs among HD and IAI patients, through week 16, was comparable. Mean intraocular pressure change from baseline, occurrence of intraocular pressure events, occurrence of APTC events or deaths, hypertension adverse events among HD and IAI patients was comparable at week 16. Mean changes in blood pressure (systolic or diastolic) through week 16 among all HD and IAI patients or patients in a dense PK sub-study was comparable.

Results—patients reaching week 44 (n=100)—values as reported at the time for patients reaching week 44. The study ended at week 44 with 100 patients. With an identical dosing regimen and slightly fewer rescue and/or PRN doses, a higher proportion of eyes receiving 8 mg aflibercept (HD) were dry in the center subfield relative to the eyes receiving 2 mg aflibercept (IAI). In addition, a change from baseline in central subfield thickness (CST) suggested better anatomic outcomes in the 8 mg HD group relative to the 2 mg IAI group. Changes in visual acuity from baseline favored the 8 mg dosing regimen (HD) over the 2 mg regimen (IAI) (+7.9 letters vs. +5.1 letters).

No new safety signals were seen and the safety profile for the HD group was comparable to that of IA. There was one case of mild iritis in the HD group that resolved with topical therapy. Changes from baseline blood pressure and intraocular pressure were similar between the groups.

Out of the 106 patients that started in the study, 100 reached the 44-week point-49 in the IAI group and 51 in the HD group. The baseline demographics of the patients in the study were majority white and having more females than males with an average age of about 77 years. The IAI and HD groups received the same mean number of injections (5.8) through week 44 with slightly more HD patients than IAI patients not receiving additional or PRN treatments.

Patients in the HD group achieved numerically superior anatomical improvements in the eye. Retinal drying (lack of fluid in the center subfield-no intraretinal fluid (IRF) and no subretinal fluid (SRF)) at weeks 16 to 44 was higher in the HD group relative to that of the IAI group. Moreover, at weeks 16 and 44, there was a greater proportion of eyes, in the HD group than in the IAI group, without fluid in the macula (no IRF and no SRF in the macula by SD-OCT). The proportion of patients in each group without IRF (dry or SRF only) at weeks 16 and 44. At weeks 16 and 44, 70% of patients in the HD group exhibited no subretinal fluid (SRF) (dry or with IRF only) whereas 51% of the IAI group patients exhibited no SRF. Through much of the 44-week trial, the HD group achieved greater mean and median reductions in central retinal thickness (CRT) from baseline (FIG. 43, FIG. 44).

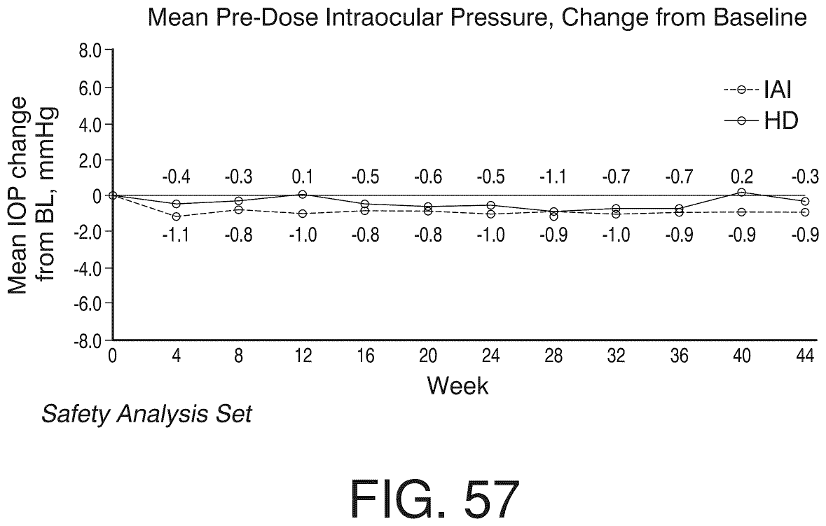

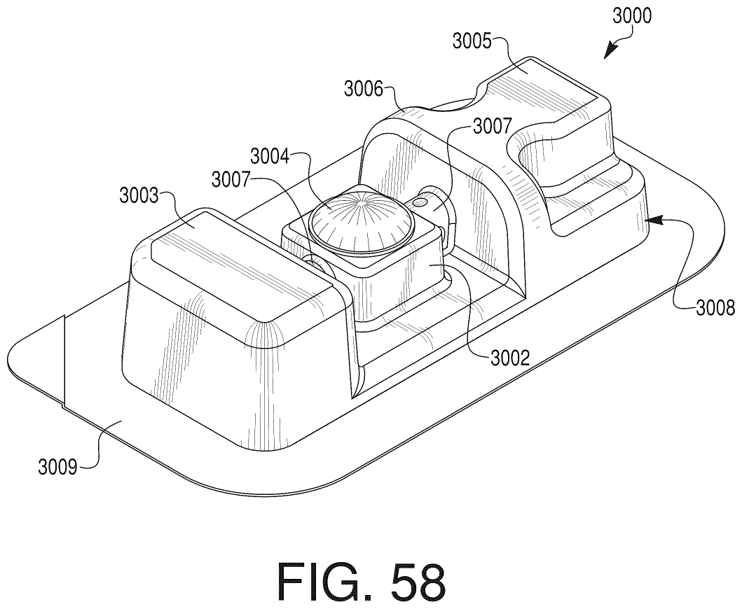

Patients in the HD group also achieved greater gains in vision. By week 44, the mean change, from baseline, in best corrected visual acuity (BCVA) was 7.9 in the HD group and 5.1 in the IAI group (FIG. 45). Fewer patients in the HD group than in the IAI group lost vision (>5, >10 or >15 letters lost) by week 44. Moreover, more patients in the HD group gained vision (>10 or >15 letters gained) (FIG. 55) by week 44. Ocular TEAEs and ocular serious TEAEs, intraocular inflammation TEAEs, intraocular pressure events, non-ocular TEAEs and non-ocular serious TEAEs were comparable between treatment groups (FIG. 46, FIG. 47, and FIG. 56, FIG. 48, FIG. 49, FIG. 50). Intraocular pressure (IOP) observed between treatment groups were also comparable (FIG. 57). Moreover, the administration of the 8 mg dose in the HD group was not observed to have an effect on the occurrence of hypertension TEAEs (FIG. 51).

### Packaging of Drug Delivery Device

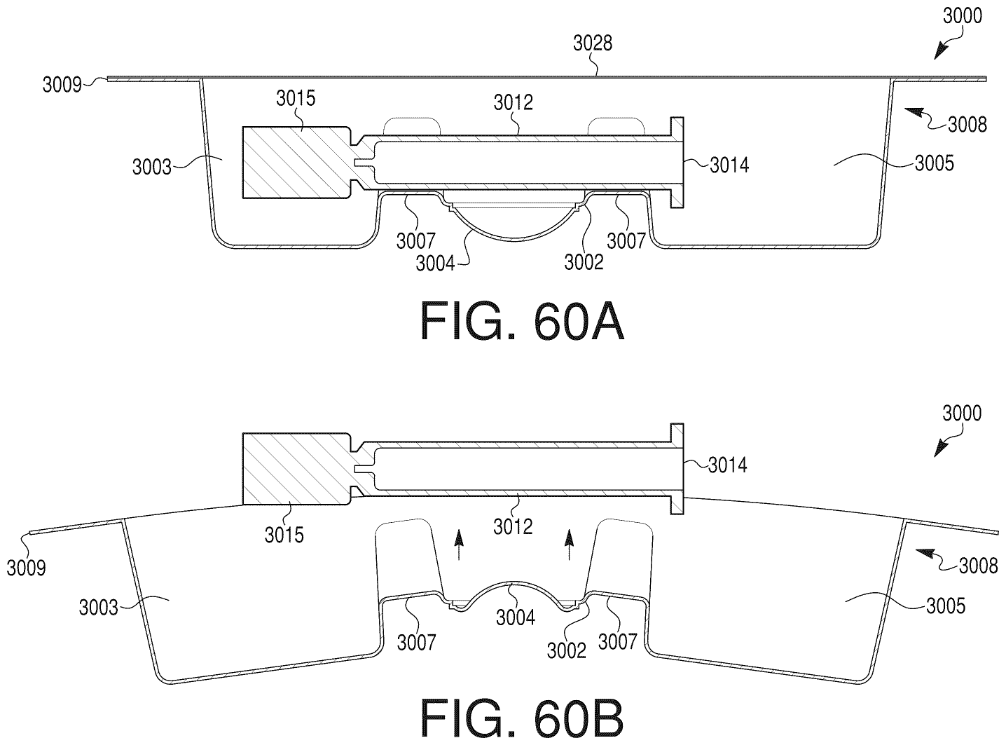

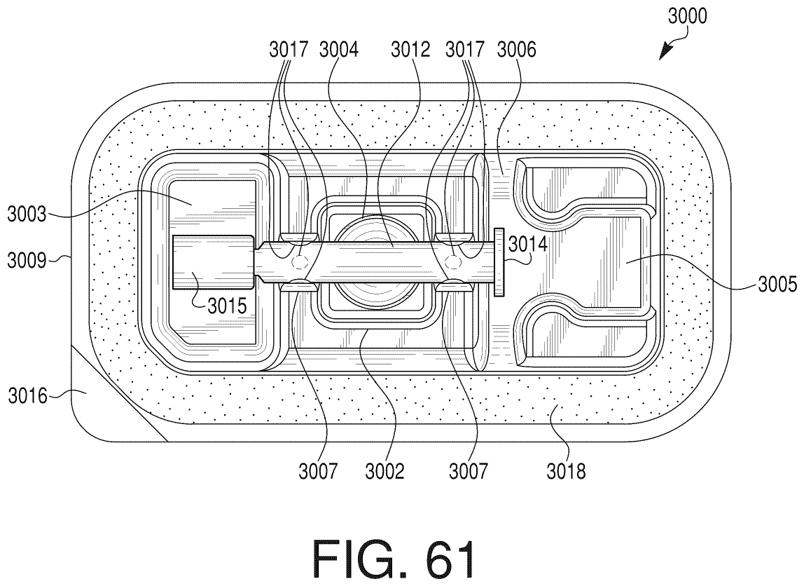

FIG. 58 shows a bottom view of exemplary packaging 3000 for a medical device. Specifically, packaging 3000 may be designed to contain a drug-delivery device such as a syringe, e.g., a pre-filled syringe (e.g., delivery device 2800). Exemplary pre-filled syringes may be used with any therapies for ophthalmic diseases, including for the treatment of patients with Neovascular (Wet) Age-related Macular Degeneration (AMD), Macular Edema following Retinal Vein Occlusion (RVO), Diabetic Macular Edema (DME), and/or Diabetic Retinopathy (DR). In particular, large molecule and small molecule antagonists of VEGF and/or ANG-2, such as aflibercept (Eylea®), ranibizumab (Lucentis®), bevacizumab (Avastin®), conbercept, OPT-302, RTH258 (brolocizumab), a pegylated designed ankyrin repeating protein (DARPin) like abicipar pegol, or RG7716 may be used with exemplary syringes. Embodiments of the present disclosure may also be used for cosmetic applications or medical dermatology, such as treatment or diagnosis of allergic responses. It should be appreciated that the syringe may be packaged within packaging 3000 after being assembled and prior to sterilization. However, the syringe may be a sterile component that is filled and assembled in an aseptic environment prior to packaging 3000. Packaging 3000 includes a tray 3008, in which the syringe is contained during storage, and a lip 3009 extending around a perimeter of tray 3008 and defining an opening in tray 3008. The top of tray 3008 (shown in FIG. 61) may be sealed with a removable cover 3028 (FIG. 60A), which may enclose the syringe within tray 3008 during sterilization and/or storage. In some embodiments, packaging 3000 may be blister packaging, i.e., packaging 3000 may be a pre-formed plastic packaging, and tray 3008 may be made from a formable web, such as a thermoformed plastic. In some embodiments, packaging 3000 may have a cavity disposed between removable cover 3028 and tray 3008 formed under pressure. The cavity may be formed under a pressure in the range from approximately 5.0 bar to 6.8 bar. In some embodiments, the cavity is formed at a temperature in the range from approximately 105° C. to 115° C. Packaging 3000 may be sealed at a temperature in the range from approximately 130° C. to 135° C. Packaging 3000 may undergo a sealing force in the range from approximately 70 kN to 75 kN. As an illustrative example of a system for sealing packaging 3000, the system may be configured to seal a plurality of units of packaging 3000, such as, approximately 105 units of packaging 3000 per minute.

Packaging 3000 may be suitable for use with an external sterilization process, e.g., a vapor hydrogen peroxide (VHP) sterilization process and/or an ethyl alcohol sterilization process. Accordingly, a medical device (e.g., a pre-filled syringe) may be packaged within packaging 3000 and then subjected to external sterilization. Sterilants, such as VHP, may not affect the medication contained within the syringe but may sterilize the exterior surface of the syringe. For example, a medical device (e.g., delivery device 2800) may be filled, assembled, packaged, and subject to sterilization within the same and/or different aseptic environments. Exemplary sterilization processes are described in PCT/US2018/021013, which is incorporated by reference herein in its entirety. Materials used in packaging 3000 may be semi-permeable to sterilizing agents to allow the sterilizing agents to traverse portions of or all of packaging 3000 to sterilize the exterior of the medical device contained within packaging 3000, as well as the interior of packaging 3000, once the medical device is sealed within packaging 3000. For example, at least 75%, at least 80%, at least 85%, at least 90%, or at least 95%, of packaging 3000 is sterilized. For example, a removable cover 3028 (FIG. 60A) may be permeable to vapor hydrogen peroxide and/or an ethyl alcohol. Cover 3028 may be formed of, e.g., Tyvek® or other suitable high-density polyethylene fibers, ethylene-vinyl acetate, and/or other thermoplastic materials. In some embodiments, tray 3008 may include an indicator to allow a user to determine whether tray 3008 and its contents have been sterilized. For example, tray 3008 may include an indicator (e.g., a tape or a label) that changes appearance in color or pattern to visually show whether a sterilization process has been performed.

Designing packaging 3000 to be used with external sterilization processes and to allow a user to remove the contents of packaging 3000 without directly handling the contents, as described herein, reduces the likelihood that sterility of the contained medical device will be compromised. As a result, packaging 3000, as described herein, may also reduce the chance that a person on whom the medical device is used will be infected by the medical device. For example, if packaging 3000 contains an ophthalmic syringe, packaging 3000 may decrease the risk of a person getting an eye infection, e.g., intraocular infections like post-injection endophthalmitis, as a result of a contaminated syringe.

An externally-sterilized syringe may be one in which any viable organisms on the surface of the syringe have been terminated or killed, making them non-viable even though they may remain. The external surface(s) of the syringe plunger rod, flange, trailing surface of the piston, and the interior of the syringe barrel behind the syringe piston, and any other external surfaces of the syringe may be externally-sterilized. In some aspects, depyrogenation and/or other removal processes may be undertaken prior to external sterilization.

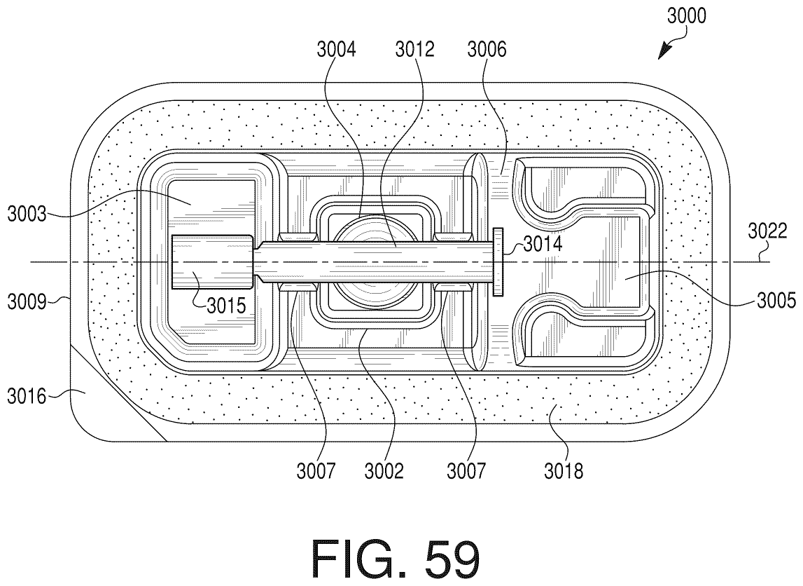

Tray 3008 may include a cavity 3003 configured to contain a distal portion of a syringe. The distal end of the syringe may include e.g., a luer lock and/or a syringe attachment to which a needle may be coupled once the syringe is removed from tray 3008 for use. In other embodiments, the needle may already be coupled to the syringe within packaging 3000 and may be further covered by a cap during storage in packaging 3000. A cavity 3002 may be configured to contain at least a portion of the syringe barrel (which may be a glass or plastic material compatible with the sterilant), and a cavity 3005 configured to contain a flange and/or plunger of the syringe. Cavity 3005 may be shaped to correspond to the shape of a flange and/or plunger of the syringe, as shown in FIG. 58. For example, flange portion 3006 of cavity 3005 may correspond to the shape of a flange. Comparing FIGS. 58, 62, 64, 66, and 71, the shape and/or sidewalls of flange portion 3006 of cavity 3005 may be rounded to contain a flange of the syringe. Comparing FIGS. 59, 63, 65, 69, and 74, an interior region of flange portion 3006 of cavity 3005 may be configured, e.g., to include rounded sidewalls, to contain a flange of the syringe. In some embodiments, flange portion 6 may be a cavity separate from cavity 3005. For example, in some embodiments, tray 3008 may include cavity 3003, cavity 3002, flange portion 3006, and cavity 3005, or combinations thereof.

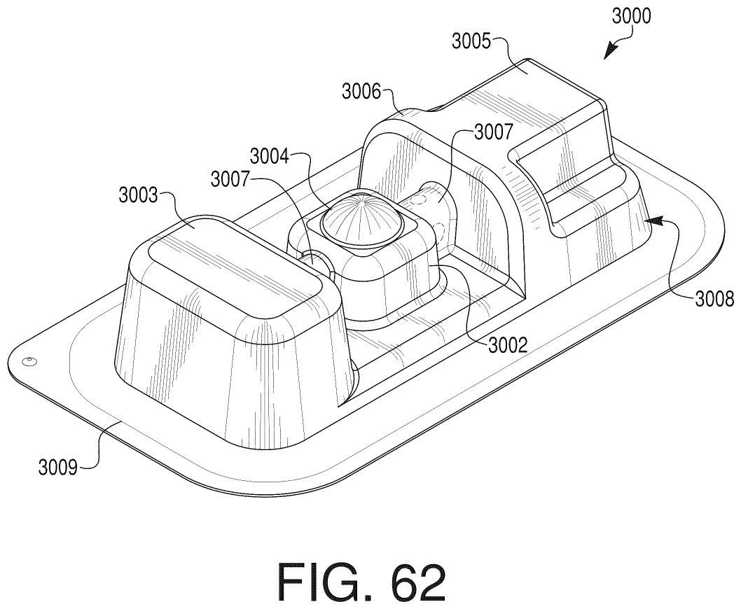

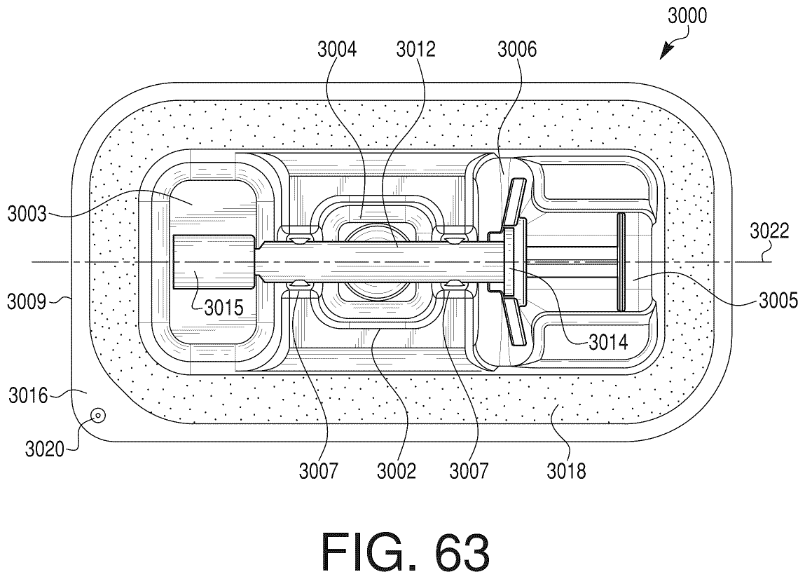

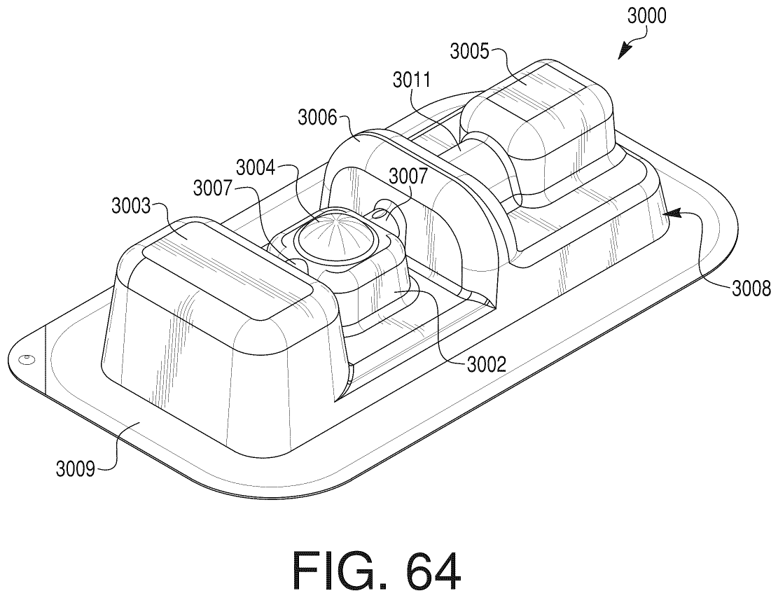

In some aspects, a syringe contained within tray 3008 may include a larger, e.g., wider plunger rod, or larger portions of a plunger rod, compared to a standard syringe, to increase the stability of a clinician's hand when pushing the plunger rod, e.g., for intravitreal injection (FIGS. 64 and 65). In such aspects, packaging 3000 may be sized and shaped to accommodate the larger plunger rod. If a syringe contained within tray 3008 includes a protective plunger stop, cover, and/or other element to prevent movement of the syringe plunger and/or dispensing of medicament or other fluid contained within the syringe, then cavity 3005 may be shaped to reflect the contours of a flange and/or plunger with the protective element in place. In other embodiments, however, cavity 3005 may be substantially rectangular, oval, or any other suitable shape, so long as an interior region of cavity 3005 defines a space large enough to contain a proximal portion of the syringe, and, if included, any accompanying protective elements. For example, FIGS. 62 and 63 show exemplary embodiments where the longitudinal side walls of cavity 3005 are generally planar and parallel to at least a portion of the plunger rod of the syringe. FIGS. 64 and 65 show exemplary embodiments where cavity 3005 may include recessed portion 3011 shaped to correspond to a shape of at least a portion of the flange or plunger, e.g., a protective plunger stop, cover, and/or other element to prevent movement of the syringe plunger and/or dispensing of medicament or other fluid contained within the syringe. For example, recessed portion 3011 may be between a main region of cavity 3005 and flange portion 3006. In exemplary embodiments, for example, FIG. 62, the shape of cavity 3005 may be sized to facilitate high speed manufacturing of packaging 3000 and/or improve material distribution of packaging 3000.

Similarly, although cavity 3003 is shown as substantially rectangular and cavity 3002 is shown as generally a square in the exemplary embodiment of FIG. 58, cavity 3003 and cavity 3002 may be of any suitable size and/or shape so as to contain a distal end of the syringe (and any cover associated with the distal end) and at least a portion of a syringe barrel, respectively. For example, cavities 3002 and 3003 may be an oval, circle, trapezoid, or a wider or narrower rectangle, or any combination thereof. Cavities 3002, 3003, 3005 may be dimensioned to provide space between the walls of the cavities and the syringe to allow a sterilant to circulate within packaging 3000 during a sterilization process in order to sterilize the internal surfaces of packaging 3000 and the external surfaces of the syringe. In some embodiments, one or more cavities 3002, 3003, 3005 may include one or more abutments (e.g., projections, protrusions, tabs, bumps, bumpers, etc.) to separate the syringe from packaging 3000 by a space and/or gap to permit a sterilant (e.g., VHP) to flow around the syringe and sterilize the exterior surfaces of the syringe positioned adjacent to the surfaces of cavities 3002, 3003, 3005. As described further below, it should be appreciated that various other features, surfaces, and/or components of packaging 3000 may include one or more abutments in addition to and/or in lieu of cavities 3002, 3003, 3005 without departing from a scope of this disclosure. Further, tray 3008 and cavities 3002, 3003, 3005 may have substantially flat surfaces on the top and bottom (e.g., cover 3028 and the base) to allow multiple packages to be stacked on top of each other during storage. Flat surfaces may also allow for consistent placement in an autoclave. That said, in some embodiments, cavities 3002, 3003, 3005 and tray 3008 may have a rounded, e.g., cylindrical shape, or any other suitable shape. Further, tray 3008 may have rounded or sharp corners and/or edges. In exemplary embodiments, for example, referring to FIG. 62, the shape and/or size of cavities 3002, 3003, 3005, and tray 3008 may be shaped and/or sized to facilitate high speed manufacturing of packaging 3000 and/or improve material distribution of packaging 3000.

In the embodiments of FIGS. 58, 62, 64, 68, and 73, narrowed portions 3007 extend between cavity 3002 and cavities 3003, 3005, fluidly coupling all three cavities. Narrowed portions 3007 are dimensioned to contain regions of the syringe barrel within them, allowing the syringe to extend into all three cavities when housed within packaging 3000, as can be seen in FIGS. 59, 63, 65, 69, and 74. For example, in FIGS. 59, 63, and 65, as described above, a plunger flange 3014 of the packaged syringe lies within a flange portion 3006 of cavity 3005, syringe barrel 3012 extends within narrowed portions 3007 and across cavity 3002, and a distal region of the syringe, at least partially covered by a guard 3015 (e.g., a luer lock), lies within cavity 3003.

Although tray 3008 is shown as including three cavities 3002, 3003, 3005, extending along a longitudinal axis 3022 (FIGS. 59, 63, 65, 69, and 74) and connected to one another by narrowed portions 3007, it is contemplated that tray 3008 may have any suitable number of cavities. For example, one cavity of substantially equal depth may be included to hold a medical device, two cavities may be included, or more than three cavities may be included. Each of the cavities (in embodiments with multiple cavities) may be connected to one another so that they may in combination contain a medical device. Or, if multiple medical devices or multiple components are housed in tray 3008, then tray 3008 may include multiple cavities not connected to one another. In such embodiments, it is contemplated that more than one of the cavities may include a projection to facilitate removal of the medical device(s) and/or component(s) from tray 3008, as will be described further below.

In the embodiment of FIGS. 58 and 59, cavity 3002 includes a dome 3004 (e.g., a projection) on a bottom portion of packaging 3000. Dome 3004 may be a flexible, hollow projection in tray 3008 that is dome-shaped and configured to facilitate removal of a syringe from packaging 3000. In this instance, dome 3004 may be open towards cavities 3002, 3003, 3005 of tray 3008. In other embodiments, dome 3004 may be a rigid projection, may have a bulbous shape, and/or may be configurable to be at least partially deformable. Dome 3004 is associated with the base of cavity 3002 so that dome 3004 is oriented beneath the syringe when the syringe is housed within packaging 3000. In the embodiment of FIG. 59, dome 3004 is aligned with a central region of syringe barrel 3012. Dome 3004 may be made of a compressible material that deforms under pressure, e.g., pressure applied by a user. As described herein, dome 3004 may be configured to flex tray 3008 in response to an application of force onto the projection, thereby at least partially deforming one or more of cavities 3003, 3004, 3005 to partially decouple the syringe (e.g., syringe barrel 3012) from tray 3008. Dome 3004 may be made of the same material as the rest of tray 3008 or may be formed of a different, e.g., a relatively more flexible material. In some embodiments, as discussed above, dome 3004 and tray 3008 may be thermoformed plastic. For example, dome 3004 and/or tray 3008 may be formed of polypropylene, polystyrene, polyethylene, polycarbonate, polyvinyl chloride, or polyethylene terephthalate glycol (PETG). A diameter of a base of dome 3004 (e.g., the widest portion of dome 3004 where it meets the base of tray 3008) may be approximately 10 mm to 20 mm, for example, approximately 10 mm, approximately 11 mm, approximately 12 mm, approximately 13 mm, approximately 14, approximately 15 mm, approximately 16 mm, approximately 17 mm, approximately 18 mm, or approximately 19 mm. Referring to FIG. 62, as compared to FIG. 58, a diameter of a base of dome 3004 may be sized in order to facilitate high speed manufacturing of packaging 3000 and/or improve material distribution of packaging 3000. In some embodiments, a height of the projection of dome 3004 from an apex of the projection to a base of the projection may be greater than a distance between the base of the projection and the syringe contained within cavities 3003, 3004, 3005 and overlying dome 3004. In further embodiments, a bulbous shape of dome 3004 may include a diameter that defines a widest portion of the bulbous shape where the projection of dome 3004 meets the base of tray 3008. It should be appreciated that dome 3004 may have a widest diameter at the base of tray 3008 and the projection of dome 3004 may extend directly outwards from the base of tray 3008 such that the widest diameter of dome 3004 is connected at the base.

FIG. 60A depicts a cross-sectional view of tray 3008 containing a syringe (e.g., an empty or a pre-filled syringe containing an ophthalmic medicament or other fluid). As discussed above, dome 3004 projects outwards from the base of tray 3008 away from the syringe when the syringe is received in tray 3008, and dome 3004 is aligned with and underneath (or over, depending on the orientation of tray 3008) syringe barrel 3012. To remove the syringe from packaging 3000, removable cover 3028 may be removed, e.g., peeled off, tray 3008 may be turned sideways or up-side-down to position an opening of the packaging towards a surface, and dome 3004 may be pushed by a user. As an illustrative example, removable cover 3028 may be peeled off from tray 3008 with a force in the range of approximately 1.6 N to 23 N. A user may push dome 3004 with one or more fingers, e.g., a thumb, towards the syringe housed within the packaging. For example, a user may use one or more fingers from a single hand of the user to push dome 3004 towards the syringe housed within the packaging 3000 for removal of the syringe therefrom. In further examples, a user may use two hands without departing from a scope of this disclosure. Additionally, packaging 3000 may be configured to eject certain components of the syringe (e.g. delivery device 2800), such as a proximal portion of the syringe, prior to other components of the syringe, such as a distal portion of the syringe, for controlling a removal of the syringe therefrom. As the user pushes on dome 3004, the packaging may flex so that a central portion of the packaging bends towards the medical device while one or more edges of the packaging bend away from the medical device. As packaging 3000 flexes (FIG. 60B), this may cause the syringe to be dislodged from the packaging. Because packaging 3000 is oriented with an opening facing gravitationally downwards (or at least perpendicular relative to a surface below), the syringe may be ejected out of packaging 3000 and onto the surface. Accordingly, pushing dome 3004 and causing packaging 3000 to flex may allow the user to remove the syringe from packaging 3000 without ever directly touching the syringe. When packaging 3000 is held upside down, the syringe may not fall out on its own, and packaging 3000 may suspend and retain the syringe until a user flexes packaging 3000, e.g., by pushing dome 3004.

When a user pushes dome 3004 to flex tray 3008, dome 3004 may deform and may at least partially invert. As dome 3004 inverts, it may or may not be configured to contact syringe barrel 3012. In some embodiments, dome 3004 may push on syringe barrel 3012 to help expel the syringe from packaging 3000. In such embodiments, a height of dome 3004 from the apex of dome 3004 to the base of the cavity from which dome 3004 extends may be greater than a distance from the base of the cavity to where the medical device (e.g., syringe) is positioned within tray 3008. As used herein, the term “apex” refers to a portion of dome 4 that is furthest away from the base of the cavity from which dome 3004 extends, irrespective of the relative orientation of dome 3004 and tray 3008. In an exemplary embodiment, the syringe may be contained within tray 3008 so that a height of dome 3004 from an apex of dome 3004 to the base of tray 3008 is approximately 5 mm to 6 mm, and wherein a distance between the base of tray 3008 and a bottom portion of the syringe overlying dome 3004 is approximately 3 mm to 4 mm. In other embodiments, however, dome 3004 may not contact syringe barrel 3012 when pressed by a user. Instead, the flexure of tray 3008 caused by pressing dome 3004 combined with orienting an opening of tray 3008 gravitationally downwards may be enough to dislodge the syringe from tray 3008 and to cause the syringe to be ejected onto the surface (preferably a sterile surface).

A user may push dome 3004 to flex tray 3008 before, during, and/or after orienting tray 3008 so that an opening of tray 3008 faces downwards or sideways toward a surface onto which the syringe is to be ejected (i.e., turning tray 3008 so that an opening faces gravitationally downwards). Orienting the tray with an opening towards the surface may allow the syringe to fall out from tray 3008 onto a suitable sterile surface without the user directly handling the syringe in order to remove the syringe from its packaging. Decreasing the amount that a user handles the syringe may decrease the possibility that the syringe and/or its contents become contaminated and infect the portion of the body that the contents of the syringe are injected into. For example, sterile ophthalmic syringes may be contained within packaging 3000, and dome 3004 and packaging 3000 may allow a user to not jeopardize sterility when removing the syringe, which may ultimately decrease the risk of eye infections, e.g., post-injection endophthalmitis, due to contaminated syringes. Decreasing handling of the syringe may also decrease the risk of accidentally discharging some of the medicament or other fluid from the syringe when removing the syringe from packaging 3000.

The location of projections like dome 3004 and/or other projection portions on tray 3008 may not only facilitate removal of the syringe but may also guide a user on how to handle certain portions of packaging 3000 when removing the syringe from packaging 3000. For example, in some embodiments, dome 3004 or other portions of packaging 3000 may include markings, indicia, and/or tactile features (e.g., ridges, bumps, or grooves) indicating to a user where to push in order to flex tray 3008 and dislodge the syringe from packaging 3000. Overall, inclusion of dome 3004 or other suitable projections in tray 3008 may decrease one or more of the likelihood of injury to a user, damage to the device, loss of sterility of the device or any medicament or other fluid therein, and/or accidental discharge of medicament or other fluid within the device.

In some embodiments, pushing dome 3004 and/or bending or flexing tray 3008 may release the syringe from a friction-fit portion of tray 3008. For example, as is shown in FIG. 61, narrowed portions 3007 may include one or more geometric features 3017 projecting into the narrowed portions to retain the sterile syringe in the cavity via friction fit. Geometric features 3017 may be located on the sidewalls of narrowed portions 3007 and/or on the bottom of narrowed portions 3007 to position syringe barrel 3012 within narrowed portions 3007. In some embodiments, it should be appreciated that geometric features 3017 and/or any other features of packaging 3000 may not be configured and/or operable to restrict, limit, prevent, and/or inhibit relative movement of the one or more components of the syringe relative to one another when housed within packaging 3000. In order to package the syringe within tray 3008 so that packaging 3000 both facilitates sterilization, e.g., decreases the area of contact with the syringe to make the most surface area of the syringe available for sterilization while still retaining, and suspending the syringe in tray 3008 when turned upside down, the syringe may be pushed into tray 3008 so that the syringe barrel 3012 slides past geometric features 3017, allowing the syringe to friction-fit within tray 3008. Once syringe barrel 3012 is pushed into place in tray 3008, geometric features 3017 may keep syringe barrel 3012 within narrowed portions 3007 and may hold the syringe within tray 3008. Pushing dome 3004 may flex tray 3008 and/or may push up syringe barrel 3012 so that syringe barrel 3012 is moved past geometric features 3017, releasing the syringe from tray 3008. For example, bending tray 3008 may force portions of tray 3008 that include geometric features 3017 to move away from the syringe and thus to release the syringe. Accordingly, dome 3004 may be used in combination with other friction-fit portions of tray 3008 to facilitate release of the syringe from tray 3008.

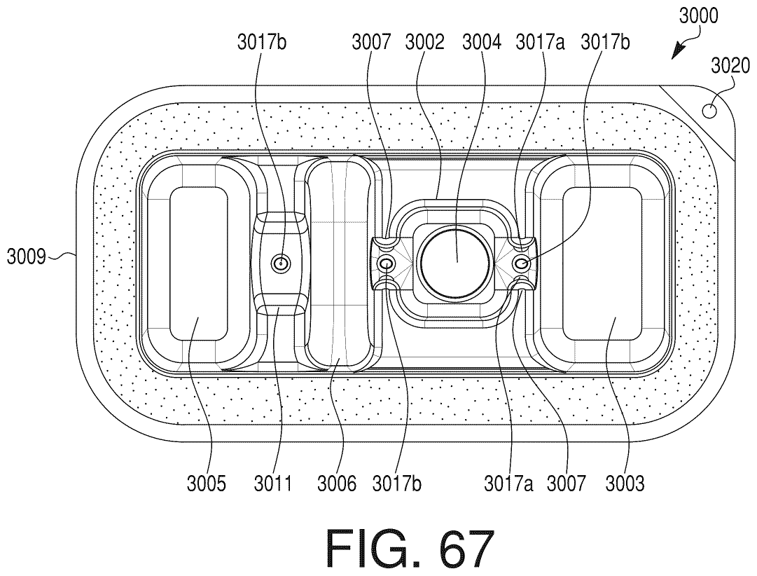

In other embodiments of the present disclosure, as shown in FIGS. 67-76, geometric features 3017 may include geometric features 3017a located on the sidewalls of narrowed portions 3007 and geometric features 3017b located on the base of narrowed portions 3007. Geometric features 3017a may be a shoulder or have a semi-circle shape that protrudes towards the syringe, while geometric features 3017b may be circular in shape, e.g., a bump or a protrusion, that protrudes towards the syringe. One or more geometric features 3017b located on the base of narrowed portions 3007 may act in conjunction with geometric features 3017a on the sidewalls of narrowed portions 3007 in order to orient the syringe so that the syringe does not directly touch the sidewalls of narrowed portions 3007 and/or of any of the other cavities, e.g., cavities 3002, 3003, 3005 when contained within packaging 3000. This may allow a sterilant within packaging 3000 to circulate within packaging 3000 and around the outer surface of the syringe. By including geometric features 3017 to hold the syringe in place while spaced apart from the internal surface of the packaging, geometric features 3017 may reduce the surface area of the syringe in contact with the inner walls of packaging 3000 and, as a result, may increase the surface area of the syringe that is exposed to a sterilant within packaging 3000.

While three geometric features 3017 are depicted in each narrowed portion 3007 of FIG. 61, it is contemplated that fewer (e.g., two) or more than three geometric features 3017 may be included in each narrowed portion 3007. Further, geometric features 3017 may be included in any suitable portion of tray 3008, including any combination of cavities, instead of, or in addition to, narrower portions like narrowed portions 3007.

FIG. 65 shows a similar delivery device (e.g., delivery device 2800) coupled to packaging 3000 as similarly described with reference to FIG. 59. Dome 3004 may be aligned with body 1220 of delivery device 2800 within packaging 3000. As discussed above, dome 3004 projects outwards from the base of tray 3008 away from body 1220 when body 1220 is received in tray 3008, and dome 3004 is aligned with and underneath (or over, depending on the orientation of tray 3008) a portion of body 1220. Body 1220 may be coupled to narrowed portions 3007 on either side of body 1220 for securing delivery device 2800 to packaging 3000. Cavity 3003 may be aligned with expulsion end 1224 of delivery device 2800 within packaging 3000. Cavity 3003 projects outward from the base of tray 3008 away from expulsion end 1224 when expulsion end 1224 is received in tray 3008, and cavity 3003 is aligned with and underneath (or over, depending on the orientation of tray 3008) expulsion end 1224. Cavities 3005 and flange portion 3006 may be aligned with portions of flange piece 2870 and plunger rod 2880 of delivery device 2800 within packaging 3000, respectively. Cavities 3005 and flange portion 3006 may project outward from the base of tray 3008 away from flange piece 2870 and plunger rod 2880 when flange piece 2870 and plunger rod 2880 are received in tray 3008, and cavities 3005 and flange portion 3006 may be aligned with and underneath (or over, depending on the orientation of tray 3008) flange piece 2870 and plunger rod 2880, respectively.

FIGS. 67-76 also show embodiments of the present disclosure that include a recessed portion 3011 between flange portion 3006 and main cavity 3005. Recessed portion 3011 may also include one or more geometric features 3017. For example, recessed portion 3011 may include one or more of geometric features 3017a and 3017b. As shown in FIGS. 67-76, recessed portion 3011 between flange portion 3006 and main cavity 3005 includes geometric feature 3017b. Geometric feature 3017b may serve the same purpose as discussed above.

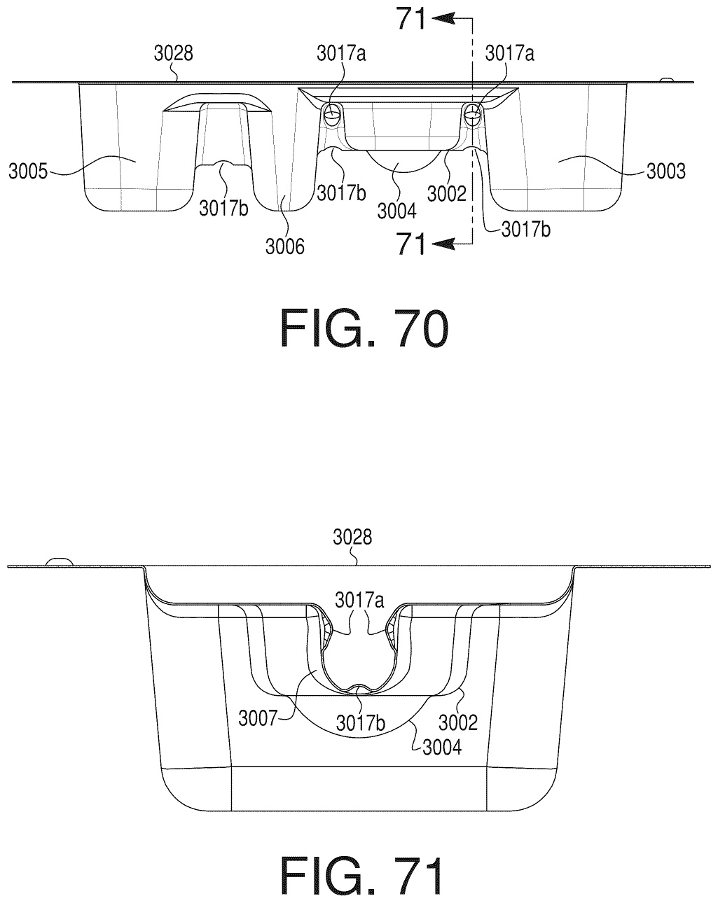

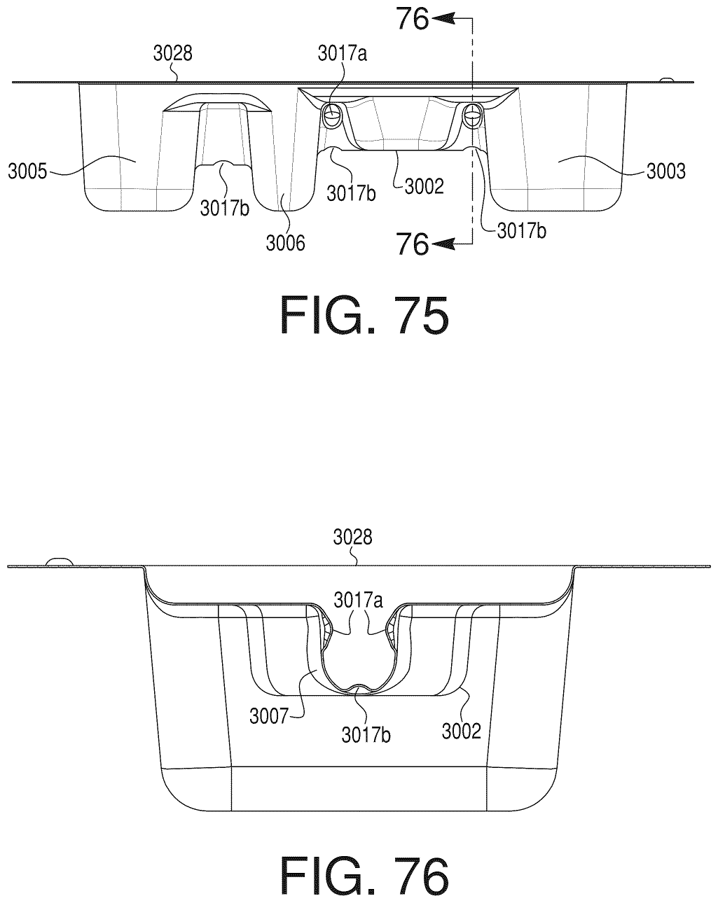

FIGS. 71 and 76 provide cross-sectional views of the embodiments shown in FIGS. 70 and 75, respectively. These cross-sectional views show the geometric features 3017a and 3017b in the narrowed portion 3007 in further detail. The first and second geometric features 3017a may be disposed opposite to each other or off set from one another (not shown).

The distance between geometric features 3017a within each of the narrowed portions 3007 (e.g., FIGS. 71 and 76) may be of any suitable distance so as to hold a portion of the syringe (e.g., a pre-filled syringe) in place. By holding one or more portions of the syringe in place, geometric features 3017a may prevent the syringe from either touching the base portions of the narrowed portions 3007 or of cavities 3002, 3003, 3005, of packaging 3000 or slipping out of packaging 3000. For example, referring to one narrowed portion 3007, narrowed portion 3007 may include a pair of sidewalls. The sidewalls may include a pair of geometric features 3017a, a first geometric feature 3017a and a second geometric feature 3017a, wherein first geometric feature 3017a and second geometric feature 3017a may have a distance that is equal to or less than the diameter or width of the portion of the syringe that is held by the geometric features 3017a. In some examples, the distance between first geometric feature 3017a and second geometric feature 3017a is equal to the diameter or width of the portion of the syringe that is held by the features. In other examples, the distance between first geometric feature 3017a and second geometric feature 3017a is less than the diameter or width of the portion of the syringe that his held by the features. In some examples, the distance between first geometric feature 3017a and second geometric feature 3017a is about 0.01 mm to about 2.0 mm less than the diameter or width of the portion of the syringe that his held by the features. In certain examples, the distance between first geometric feature 3017a and second geometric feature 3017a is about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm less than the diameter or width of the portion of the syringe that is held by the features. In some embodiments, the width of the portion of the syringe that is held by the features includes the outer diameter of the syringe alone or the outer diameter of the syringe together with a label that has been adhered to the syringe.

In some embodiments, the diameter or width of the portion of the syringe that is held by the geometric features 3017a is about 6.85 mm without a label adhered to the syringe. In such embodiments, the distance between first geometric feature 3017a and second geometric feature 3017a can be 6.85 mm±2.0 mm, including any fractional increment thereof. In other embodiments, the diameter or width of the portion of the syringe that is held by the geometric features 3017a is about 6.92 mm with a label adhered to the syringe. In such embodiments, the distance between first geometric feature 3017a and second geometric feature 3017a can be 6.92 mm±2.0 mm, including any fractional increment thereof.

In alternative embodiments, the distance between first geometric feature 3017a and second geometric feature 3017a may range from about 4 mm to about 20 mm, about 4.25 mm to about 19 mm, about 4.5 mm to about 18 mm, about 4.75 mm to about 17 mm, about 4.75 mm to about 16 mm, about 5 mm to about 15 mm, about 5.25 mm to about 14 mm, about 5.5 mm to about 13 mm, about 4.25 mm to about 15 mm, about 4.5 mm to about 10 mm, about 4.75 mm to about 9 mm, about 5 mm to about 7.5 mm, about 5.25 mm to about 7.25 mm, about 5.5 mm to about 7.2 mm, about 5.55 mm to about 7.1 mm, about 6.6 mm to about 7.1 mm, about 6.7 mm to about 7.05 mm, about 6.75 mm to about 6.95 mm, about 6.8 mm to about 6.9 mm, about 6.8 mm to about 7.1 mm, about 6.85 mm to about 7.1 mm, or about 6.85 mm to about 7.05 mm. In some examples, the first geometric feature 3017a and second geometric feature 3017a may have a distance of about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, or about 9.0 mm. In other examples the first geometric feature 3017a and second geometric feature 3017a may have a distance of about 5 mm±0.5 mm, about 6 mm±0.5 mm, about 7 mm±0.5 mm, about 8 mm±0.5 mm, about 9 mm±0.5 mm, or about 10 mm±0.5 mm.

In embodiments where the sidewalls of recessed portion 3011 include a first geometric feature 3017a and a second geometric feature 3017a, the distance between first geometric feature 3017a and second geometric feature 3017a in recessed portion 3011 may be the same as the distance between first geometric feature 3017a and second geometric feature 3017a in narrowed portion 3007. In other embodiments, the distance between first geometric feature 3017a and second geometric feature 3017a in recessed portion 3011 is different than the distance between first geometric feature 3017a and second geometric feature 3017a in narrowed portion 3007.

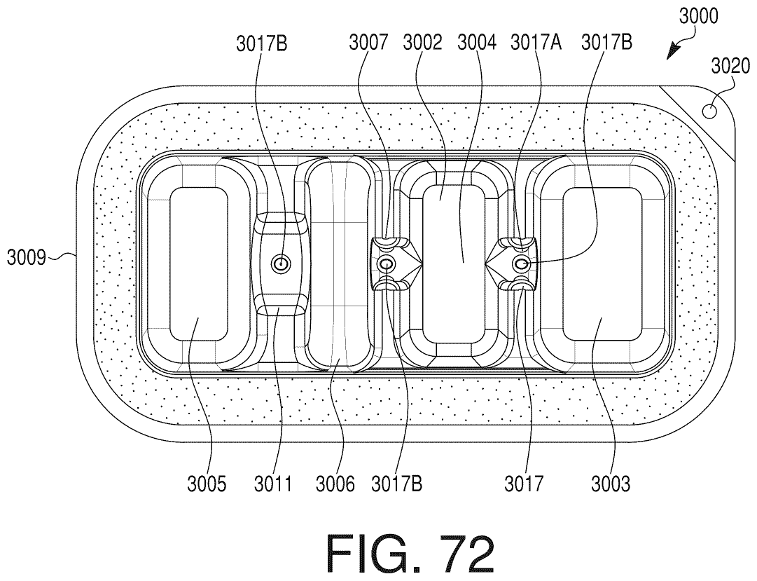

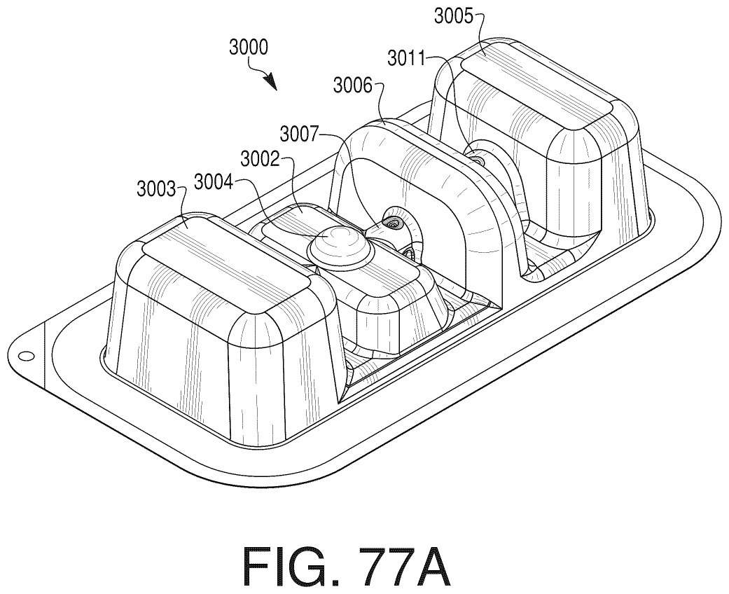

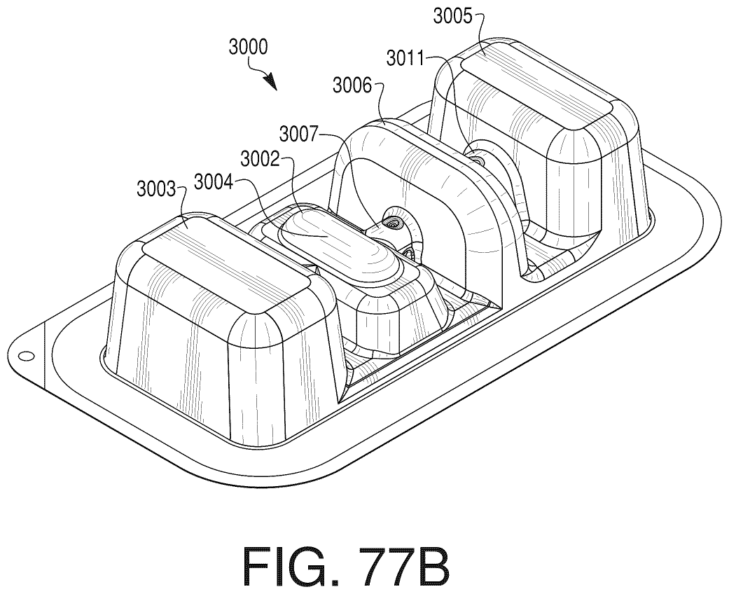

Although reference is made herein to a dome-shaped portion, it should be understood by one of ordinary skill in the art that a projection on tray 3008 may have any suitable shape so long as it is capable of being pushed to flex tray 3008 and/or to be compressed to an inverted position. For example, as discussed above, the embodiments of FIGS. 72-76 may include one or more cavities having an elongated shape. The one or more cavities may have a projection with an elongated shape, i.e., a rectangular shape, to correspond to the shape of the corresponding cavity. Further, although tray 3008 is depicted as including three cavities 3002, 3003, 3005, connected to one another by narrowed portions 3007, it is contemplated that tray 3008 may have any suitable shape with one or more cavities to house a medical device. For example, although embodiments of the present disclosure are drawn to packaging for a syringe, e.g., a pre-filled syringe, it is to be understood that projections, such as dome 3004, may be used in conjunction with packaging for any suitable medical device and/or consumer good. Dome 3004 may have any size and/or shape so as to facilitate removal of the syringe from the packaging. Dimensions of dome 3004 are discussed in the present disclosure. For example, dome 3004 may have a generally circular shape (FIG. 77A) or a generally rectangular shape (FIG. 77B). Accordingly, tray 3008 may be shaped to contain other types of medical devices. Additionally, multiple projections, e.g., multiple domes 3004, may be incorporated into tray 3008 to facilitate removal of a medical device therein.

As described above, the use of dome 3004 in packaging, e.g., blister packaging, to release a syringe housed within the packaging may promote sterility, dose accuracy, and/or safety of the syringe and the user because it may decrease the amount that a user has to directly touch the syringe prior to administration of the syringe contents to a person. This may in turn decrease the risk of accidentally discharging the syringe contents prior to administration of the medicament or other fluid to a person and reduce the incidence of infection to the person.

In some embodiments, cavities 3002, 3003, 3005, may not include a projection. For example, referring to FIGS. 72-76, a user may push against one of cavities 3002, 3003, 3005, to flex tray 3008 and so that the syringe is moved past geometric features 3017, releasing the syringe from tray 3008. In another example, a user may remove cover 3028 and then to preserve sterility of the syringe, the user may grip onto a portion of the syringe held in one or more of narrowed portions 3007 to release the syringe from geometric features 797. For example, referring to FIG. 74, the user may reach into tray 3008 and grip onto a portion of the syringe held at narrowed portion 3007 between cavities 3002 and 3003, or between cavities 3002 and 3005, or within the cavity 3002. The user may then pull the syringe until it is released from geometric features 3017a and/or 3017b.

Referring back now to FIGS. 58 and 59, packaging 3000 may also include a lip 3009 extending around at least a portion of tray 8 and defining an opening in tray 3008. Lip 3009 may allow for a removable, e.g., peelable, cover 3028 to be sealed over the top opening of tray 3008 to contain the medical device within tray 3008 during storage. An adhesive 3018, e.g., a glue, paste, film, tape, pressure-sensitive material, and/or cold adhesive, may be used to seal removable cover 3028 to lip 3009, thereby sealing an entirety of packaging 3000 simultaneously. For example, adhesive 3018 may include ethylene vinyl alcohol (EVA). Adhesive 3018 may be compatible for use with the type of external sterilization performed that may be performed on packaging 3000. The width of adhesive 3018 may be altered depending on the size of the top opening of tray 3008, e.g., an increase in size of the top opening of tray 3008 will decrease the width of adhesive 3018. A corner of lip 3009, e.g., corner 3016 in FIG. 59, may be free of adhesive 3018 to allow a user to lift removable cover 3028 off of corner 3016 to remove the cover from tray 3008. As shown in FIGS. 63 and 65, corner 3016 may include a tab 3020 (or other projection or indentation) to allow a user to lift removable cover 3028 off of corner 3016 to remove cover 3028 from tray 3008. Tab 3020 may be included on corner 3016 of lip 3009 or removable cover 3028. In exemplary embodiments, referring to FIGS. 59 and 63, the type, shape and/or size of lip 3009 or tab 3020 (or other projection or indentation) may be configured to facilitate high speed manufacturing of packaging 3000 and/or improve material distribution of packaging 3000. Removable cover 3028 may be made of any suitable material, e.g., foil paper, plastic, etc., and may be permeable to a suitable gaseous sterilant, such as vaporized hydrogen peroxide, ethyl alcohol, nitrous oxide, or any appropriate sterilant in the art. Removable cover 3028 may include not more than 10 parts per billion (ppb) of Vinyl chloride monomer (VCM). Removable cover 3028 may include a thickness greater than or equal to 483 microns and less than or equal to 533 microns.

Packaging 3000 may have a length extending along longitudinal axis 3022 of approximately 138 mm+/−10 mm, and a width extending perpendicular to longitudinal axis 3022 of approximately 75 mm+/−5 mm. Tray 3008 may have a length extending along longitudinal axis 3022 of approximately 110.0 mm+/−1.25 mm, and a width extending perpendicular to longitudinal axis 3022 of approximately 45.4 mm+/−1.0 mm.

During use, a user may first remove removable cover 3028 from tray 3008, e.g., by peeling cover 3028 off of lip 3009, to expose an opening of tray 3008. The user may then orient the opening of tray 3008 sideways or up-side-down so that the opening faces a surface onto which the medical device (e.g., pre-filled ophthalmic syringe) is to be ejected. Subsequently or simultaneously, the user may push on dome 3004 (or other projection), which may flex tray 3008 so that a central portion of the packaging bends towards the medical device while one or more edges of the packaging bend away from the medical device, such as by using a single or both hands. Pushing dome 3004 may cause dome 3004 to at least partially invert. In some embodiments, an inverted portion of dome 3004 may contact the medical device (e.g., syringe barrel 3012), while in other embodiments, dome 3004 may never contact the syringe. Pushing dome 3004 and flexing tray 3008 may thus eject the medical device from tray 3008 so that the medical device lands on the surface towards which the opening of tray 3008 was oriented. A suitable sterile surface may include, e.g., a sterile table, tray for holding medical devices, cloth, liner, or any other suitable sterile surface. The combination of pushing dome 3004, inverting dome 3004, bending tray 3008, and/or turning tray 3008 sideways or up-side-down so that the base of tray 3008 is oriented above the syringe and above the sterile surface may allow a user to remove the syringe without actually touching the syringe directly.

In some embodiments, pushing dome 3004 and flexing tray 3008 may also release the medical device from a friction-fit portion of tray 3008, e.g., one or more geometric features 3017. Turning tray 3008 may allow the syringe to fall out of tray 3008 once it is released from tray 3008 (e.g., via the inversion of dome 3004 and/or bending). In embodiments that include multiple domes 3004 (or multiple other projections), multiple domes 3004 may be pushed to release the medical device from tray 3008 and/or to flex different portions of tray 3008.

In the case of packaging for a pre-filled syringe (e.g., delivery device 2800), once the syringe is released from tray 3008, any additional packaging on the syringe (e.g., a distal guard or luer lock and/or plunger guard) may be removed from the syringe. In some embodiments, the syringe may be contained in packaging 3000 without a needle, and a needle, e.g., a stake needle, may be attached to a distal end of the syringe, e.g., via a needle attachment. The contents of the syringe (e.g., large molecule and/or small molecule antagonists of VEGF and/or ANG-2, such as aflibercept (Eylea®), ranibizumab (Lucentis®), bevacizumab (Avastin®), conbercept, OPT-302, RTH258 (brolocizumab), a pegylated designed ankyrin repeating protein (DARPin) like abicipar pegol, or RG7716) may then be injected into an eye of a recipient.

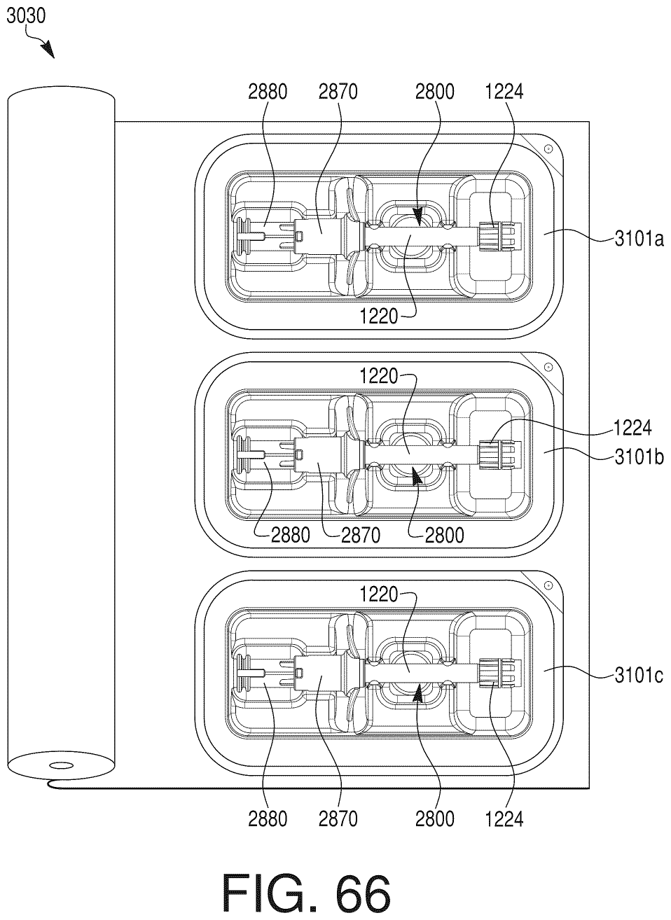

As described above, components of packaging 3000, and the size and/or shape of such components may be configured to facilitate high speed manufacturing of packaging 3000 and/or improve material distribution of packaging 3000. FIG. 66 is an exemplary embodiment showing a portion of the manufacturing process of packaging 3000, where for example, packaging 3101a, 3101b, and 3101c, are sealed with material 3030 to form removable cover 3028 (FIG. 60A), which may be, e.g., Tyvek® or other suitable high-density polyethylene fibers, ethylene-vinyl acetate, and/or other thermoplastic materials.

As described above, in some aspects of the present disclosure, after delivery devices are placed into the formed blisters (e.g. tray 3008), a blister lidding (e.g., removable cover 3028) may be affixed to the blister to seal the blister. The blister lidding may include pre-applied heat-activated adhesive, where the adhesive is configured to seal the lidding to the body of the blister. The blister lidding may be rolled over the one or more blister cavities of a blister pack, and heat-sealed around a perimeter of the pack, or around perimeters of the individual blister cavities. The blister lidding may comprise a flashspun high-density polyethylene polymer, such as, for example, those sold under the Tyvek® tradename. As used herein, Tyvek® may refer to a flashspun high-density semi-permeable polyethylene polymer.

### Systems & Methods of Sterilization

Embodiments of the present disclosure further relate to systems and methods for the use of vaporized sterilants in sterilization processes, such as processes for sterilizing medical products. For example, embodiments of the present disclosure may relate to systems and methods for the terminal sterilization of medical products using vaporized hydrogen peroxide (VHP). More particularly, embodiments of the present disclosure may relate to, e.g., systems and methods for the terminal sterilization of medical products, such as pre-filled syringes (PFS).

Some aspects of the present disclosure may be used in combination with one or more aspects of WIPO Publication No. WO 2018/182929, filed Mar. 6, 2018, and WIPO Publication No. WO 2022/217192, filed Mar. 30, 2022, each of which is incorporated by reference in its entirety.

Medical products included in a sterilization load of a terminal sterilization process may be positioned in packaging. For example, a delivery device may be filled with a medicament and packaged in a container, envelope, blister pack, or other packaging structures. Containers, envelopes, blister packs, or other packaging structures may include a plurality of pre-filled delivery devices. Examples of packaging structures that may be used with the systems and methods described in this disclosure include those disclosed in U.S. Pat. No. 11,160,918, filed Jul. 29, 2020, U.S. Patent Publication No. 2023/0248898, filed Feb. 3, 2023, and U.S. Patent Publication No. 2024/0416028, filed Jun. 14, 2024, each of which is incorporated by reference in its entirety.

In some embodiments, devices according to the present disclosure may be manufactured, packaged, filled, and/or otherwise prepared according to processes relevant to the products (e.g., drug products) of which they may be a part. For example, in some embodiments, devices according to the present disclosure may be sterilized, either before or after being filled and/or packaged. For example, in some embodiments, devices according to the present disclosure may be filled and packaged in, e.g., blister packaging, and/or may be terminally sterilized using any suitable method in the art. For example, devices according to the present disclosure may be terminally sterilized using a chemical sterilization method, such as a method including ethylene oxide or hydrogen peroxide (e.g., vaporized hydrogen peroxide). In some embodiments, devices according to the present disclosure may be terminally sterilized using methods described in, e.g., International Application No. PCT/US2018/021013, filed Mar. 6, 2018, which is incorporated by reference herein in its entirety.

In some embodiments, devices according to the present disclosure may undergo a process of packaging and sterilization as described in detail herein. As an illustrative example, the process of packaging and sterilizing may include at least the following steps: labeling and assembling the device; blister packaging of the labeled and assembled device; sterilization of the exterior of the device within the sealed blister package by vaporized hydrogen peroxide (VHP); and packaging the sterilized blister package into a carton with approved product literature. In some embodiments, the foregoing steps may be independent of the batch size of the devices.

In the example, prior to the step of sterilizing the device with VHP, the packaged device may be stored at 2-8° C. to be refrigerated. The time out of refrigeration of the device may be monitored and recorded. The packaged device may be arranged in racks stacked vertically in specialized carts prior to sterilization. To begin sterilizing the packaged device, the carts may be loaded into a sterilizing chamber. In some embodiments, the maximum and/or minimum load of the sterilization chamber may include a plurality of packaged devices in each cart, such as, for example, about 76 devices. In the embodiment, the maximum load may include approximately 18 full racks in each cart. The maximum load may include approximately 8 carts per load being sterilized. The maximum and/or minimum load may include a plurality of packaged containers per load, such as, for example, approximately 10,944. In some embodiments, the maximum load may include approximately 1 full rack in each cart and approximately 608 packaged containers per load. After the loaded carts are pushed into the VHP chamber, the chamber door may be closed and the production cycle setting is loaded, verified, and the execution of the sterilization cycle starts in accordance with the methods described in further detail herein.

It should be understood that sterilization cycle parameters may be pre-programmed in a sterilizer software such that the following processes may be run automatically, such as by a controller. In other words, the sterilization equipment may be configured to execute an automated cycle via the controller. At the start of a sterilization cycle, a chamber leak test may be conducted to ensure the chamber is fully closed with no leaks prior to injection of VHP for sterilization. In the chamber leak test, after the VHP chamber is closed, all chamber valves may be closed and a vacuum in the chamber is monitored. If the leak rate is within acceptable limits, the system is deemed to be leak tight. In some embodiments, the leak test is conducted for approximately 5 minutes and requires approximately 2 minutes of stabilization time. In further embodiments, the vacuum level is set to approximately 500 mbar and the acceptable total leak is approximately 13 mbar. In the chamber leak test, the vacuum leak rate of the chamber is measured to test the integrity and tightness of sealed openings.

The load may be preconditioned after a leak test is performed to remove moisture from the chamber prior to injecting VHP for sterilization, and allow the PFS load to equilibrate to conditions inside the chamber prior to injecting VHP for sterilization. The load conditioning phase may progress through a series of pressure pulses, first pulling vacuum and then breaking vacuum with dry, compressed air. For example, to precondition the packaged device to be sterilized, the chamber may be partially evacuated and filled with dry air pulses of approximately twelve times to lower humidity and allow time for the device to equilibrate to the temperature conditions inside the chamber, thereby helping to prevent excessive condensation. During the preconditioning phase, the vacuum level may be set to approximately 500 mbar and the vacuum break point may be set to approximately 700 mbar.

To sterilize the packaged device, the packaged device may be exposed to VHP in a series of injection pulses. This phase controls and ensures adequate dosing of VHP, such as from a VHP injection panel, into the chamber via a series of pressure pulses. The phase also ensures adequate contact time for the VHP inside the blister packs via post-injection and post-transition hold times during pressure pulses. For example, the packaged device may be exposed to VHP by approximately 12 injection pulses. During each exposure, the chamber is partially evacuated and a set amount of liquid hydrogen peroxide solution is delivered to a heated vaporizer. Vaporized hydrogen peroxide may be drawn into the chamber and recirculated through an external blower to create turbulence to enhance the penetration of the H₂O₂ through the packaging.

During a first exposure, approximately 2 pulses of VHP may be supplied to the chamber. During the first exposure, the vacuum level of the chamber may be set to approximately 500 mbar at a rate of 150 g H₂O₂ per pulse. After the first exposure, the load may be held in the chamber for a post-injection hold time of approximately 1 minute. After the first post-injection hold time, dry air is added to break vacuum to the transition pressure of approximately 940 mbar for approximately 4 minutes for a post-transition hold time.

During a second exposure, approximately 3 pulses of VHP may be supplied to the chamber. During the second exposure, the vacuum level of the chamber may be set to approximately 500 mbar at a rate of 75 g H₂O₂ per pulse. After the second exposure, load may be held in the chamber for a post-injection hold time of approximately 1 minute. After a second post-injection hold time, the chamber may be partially evacuated again to the transition pressure of approximately 940 mbar for approximately 4 minutes for the post-transition hold time and each injection sequence is executed per the cycle recipe.

During a third exposure, approximately 7 pulses of VHP may be supplied to the chamber. During the third exposure, the vacuum level of the chamber may be set to approximately 600 mbar at a rate of 50 g H₂O₂ per pulse. After the third exposure, load may be held in the chamber for a post-injection hold time of approximately 1 minute. After a second post-injection hold time, the chamber may be partially evacuated again to the transition pressure of approximately 900 mbar for approximately 10 minutes for the post-transition hold time. In the embodiment, the VHP sterilization process described herein may ensure a 6-log microbial reduction inside sealed blister packs (e.g., packaging 3000) containing a prefilled syringe (e.g. delivery device 2800) while demonstrating that exposure to the sterilizing condition does not impact the safety, identity, strength, purity, or quality (SISPQ) of the drug product stored in the prefilled syringe.

After sterilization, the sterilization chamber may be aerated to allow removal of VHP from the blister packs and sterilization chamber after completion of the VHP exposure (sterilization) phase described above. During the aeration steps, the chamber is vented and dry compressed air flows are controlled to reduce the VHP concentration in the chamber to levels suitable for personnel. During the aeration process, VHP is converted to water and oxygen using a catalytic converter process. The chamber may then be equalized with dry air. The catalytic converter step may be performed in two waves: a first step may be for exhausting the hydrogen peroxide, and a second may be for drying the chamber and contents.

In some embodiments, the aeration process may include approximately 5 steps. In a first aeration step, approximately 1 pulse of air may be supplied to the chamber at approximately 500 mbar. The vacuum may have a break point of approximately 940 mbar. The exhaust may be on for approximately 35 minutes. During the first aeration step, there may be no recirculation heating and/or recirculation when the vacuum is on. Clean air may be supplied to the chamber when the vacuum is on. During a second aeration step, approximately 1 pulse of air may be supplied to the chamber at approximately 500 mbar. The vacuum may have a break point of approximately 700 mbar. The vacuum may not have a hold time period (i.e., 0 minutes). During the second aeration step, there may be recirculation heating and/or recirculation when the vacuum is on. The chamber may have an exhaust period of approximately 0 minutes. Clean air may be supplied to the chamber when the vacuum is on.

During a third aeration step, approximately 3 pulses of air may be supplied to the chamber at approximately 500 mbar. The vacuum may have a break point of approximately 940 mbar. The vacuum may not have a hold time period, and may include an exhaust time of about 15 minutes. During the third aeration step, there may be recirculation heating and/or recirculation when the vacuum is on. Clean air may be supplied to the chamber when the vacuum is on. During a fourth aeration step, approximately 1 pulse of air may be supplied to the chamber at approximately 500 mbar. The vacuum may have a break point of approximately 600 mbar. The vacuum may not have a hold time period. The chamber may have an exhaust period of approximately 0 minutes. During the fourth aeration step, there may be recirculation heating and/or recirculation when the vacuum is on. Clean air may be supplied to the chamber when the vacuum is on.

During a fifth aeration step, approximately 1 pulse of air may be supplied to the chamber at approximately 900 mbar. The vacuum may have a break point of approximately 940 mbar. The vacuum may not have a hold time period. During the fifth aeration step, there may be recirculation heating and/or recirculation when the vacuum is on. The chamber may have an exhaust period of approximately 15 minutes. Clean air may not be supplied to the chamber when the vacuum is on. The carts previously loaded into the sterilizing chamber may be removed and stored at approximately 2° C. to 8° C. if final packaging does not occur immediately following sterilization cycle completion. In some embodiments, devices according to the present disclosure may be packaged at secondary locations from where the devices are assembled.

Devices according to the present disclosure may undergo a labeling and assembly process. In some examples, an assembly machine may be used to assemble the device packaging by packing one syringe (e.g., delivery device 2800) and one blister package (e.g. packaging 3000) into each carton; the cartons may be verified by their contents and labeled. The assembly machine may consist of “stations” driven by mechanical linear cams controlled by a programmable logic controller (PLC). Format recipes may control machine settling and process parameters. Prior to operating the assembly machine, a start-up verification may be performed for the equipment and components involved in the assembly process at the beginning of every assembly run to confirm that all the stations' sensors and setting parameters on the assembly machine are functioning correctly. Subsequent in-process challenges may also be performed during processing per procedure at selected stations.

The devices may be loaded onto the infeed systems supplying the assembly machine. After getting inspected, the devices may be transferred individually onto assembly pallets that transport the syringes through the remaining labeling and assembly stations. Within the labeling station, a lot number and expiration date may be printed on a label and inspected. Acceptable labels may be automatically applied to the syringe. The labeled devices may be then transported to the finger flange application station where the finger flange may be mechanically assembled onto the syringe barrel/body. Following finger flange application, the syringes are transported to the plunger rod insertion station. Within the plunger rod insertion station, the orientation of the plunger rods may be adjusted to align with the finger flange opening or “keyway” and checked by a sensor for correct orientation prior to insertion into the syringe of the device.

Detection systems may monitor quality attributes and process parameters on each individual syringe within all the assembly stations, as described in detail herein. Any syringe or sub-assembly of the device that fails to meet predefined acceptance criteria may be automatically rejected and removed from the acceptable product flow-path. A device rejected at a critical monitoring station, or devices consecutively rejected at any monitoring station, may create an alarm condition requiring acknowledgement prior to resuming operation. Verifications for select quality attributes of the devices may be performed throughout production and results may be recorded. The acceptable assembled devices may then be transferred to the blistering (e.g. packaging) process or returned to storage at approximately 2° C. to 8° C.

The devices may be packaged at a controlled room temperature. In preparation for processing, rolled filmstock and/or blister lidding rolls may be loaded into the packaging system for packing devices in a blister pack, such as those shown and described herein. The blister tray that may hold the assembled syringe may be created by thermoforming rolled filmstock using automated equipment. Following placement of the assembled devices into the formed blister cavities, blister lidding rolls with a pre-applied heat-activated adhesive and pre-printed artwork may be rolled over the thermoformed cavities and heat-sealed around the perimeter of the individual blister cavities. The integrity of the blister package is tested after VHP sterilization during the release of the drug product (DP). In some embodiments, packaged devices including blister packs containing assembled syringes filled with aflibercept drug product may be stored at 2° C. to 8° C. prior to sterilization by VHP, if storage in between steps is necessary.

The VHP sterilization process may be utilized to sterilize the external surfaces of the assembled device (e.g., delivery device 2800) and the interior surfaces of a blister package (e.g., packaging 3000), such as in accordance with the methods shown and described herein. In some embodiments, sterilization may occur in a closed chamber and each production sterilization cycle may consist of one or more of the following phases: leak testing; pre-conditioning; VHP exposure (sterilization); and/or aeration. The VHP exposure phase may consist of three VHP sub-exposures with a total of approximately 12 injection pulses of varying amounts of VHP. In some embodiments, the VHP consists of aqueous H₂O₂ in a concentration of approximately 5%.

As an illustrative example, a first exposure may include approximately two pulses of approximately 150 g of VHP in every pulse. A second exposure may include approximately three pulses of approximately 75 g of VHP in every pulse. A third exposure may include approximately seven pulses of approximately 50 g of VHP in every pulse. Once the sterilization program has completed, the chamber may be unloaded and, if necessary, the sterilized packaged devices are stored at approximately 2° C. to 8° C. until final packaging into cartons occurs.

In some embodiments according to the present disclosure, an in-process controls (IPCs) strategy for an 8 mg aflibercept PFS label and assembly, blister packaging, and VHP sterilization process may be established based on holistic information derived from one or more of the device and process development understanding, microbial and stability studies, risk assessments, characterization studies, historical PFS experience, subject matter expertise, and/or established process controls at the packaging site, as well as the 8 mg aflibercept PFS process qualification and validation studies. The device labeling and assembly step may have a critical process parameter of a plunger rod assembly force onto a finger flange and/or syringe barrel being less than or equal to approximately 15 N, as described in further detail herein.

In further embodiments, the force limit setting may be a recipe controlled parameter. The assembly process may be controlled through movement of mechanical grippers, which are pre-programmed to specific travel distances. For example, an electromechanical sensor may monitor the assembly force for every unit produced on the equipment. Assemblies which may exceed the force limit may be automatically rejected. In some embodiments, the equipment may generate an alarm if consecutive rejects are detected. Maintaining the plunger rod assembly force may ensure adequate force is applied during the assembly step of the plunger rod insertion, which can affect the container closure integrity of the primary container (e.g., body 1220) and the intended deliverable volume of the device.

In some embodiments, the blister pack sterilization step may have multiple exemplary critical process parameters including but not limited to: a total H₂O₂ dose, a maximum load probe temperature, a minimum pressure, and a sterilization phase duration. In some embodiments, the total H₂O₂ dose may be greater than approximately 750 g to ensure adequate dosing of VHP to sterilize the chamber load. In some embodiments, the routine manufacturing dose of H₂O₂ may be increased to a total of approximately 825 g as an added assurance of the sterilization efficacy during routine processing.

In some embodiments, the maximum load probe temperature may be greater than approximately 35° C. to ensure the high temperature limit for the drug product is not exceeded during sterilization cycle. The maximum load probe temperature is classified as a critical process parameter because of its impact on the drug product purity. The minimum pressure being greater than approximately 480 mbar may ensure the pressure differential between interior and exterior environments does not cause adverse impacts to the syringe, such as stopper movement. The sterilization phase duration may be greater than at least 3 hours to ensure adequate contact time for VHP within the load being sterilized to achieve sterilization.

The sterilization cycle may have at least one in-process control, such as a chemical indicator (CI) reactivity and biological indicator (BI) result. CIs may be used to ensure exposure of the VHP within the sterilization chamber. For example, the CIs may contain an indicator that has been specially formulated to be responsive to VHP during sterilization cycles. The CIs change color when exposed to high concentrations of VHP for extended durations. During testing, a successful CI result required observation of full color change. In some embodiments, approximately 100% color change may be required to evaluate the chemical indicator reactivity. Evaluating the color indicator reactivity ensures exposure to the sterilant (H₂O₂) within the sterilization chamber and throughout the load for the sterilization cycle. A color change ensures operational reliability and consistency.

BIs may be used to ensure VHP effectiveness (6-log microbial reduction) within the sterilization chamber. The BIs may use Geobacillus stearothermophilus as a test organism, and may have a mean population of 10⁶. Geobacillus stearothermophilus spores demonstrate high resistance to VHP, and thus were considered suitable for use in this application. BIs may be tested in a lab for growth after being subjected to the sterilization cycle. In some embodiments, no growth or confirmed approximately six-log reduction in sterility assurance level (SAL) may be required to ensure effectiveness of the sterilant (H₂O₂) within the sterilization chamber and throughout the load for the sterilization cycle. The total H₂O₂ dose may be classified as a critical process parameter because of its impact on the sterility of the blister package and the external surfaces of the pre-filled syringe. In some embodiments, the VHP sterilization may be fully automated as described herein.

In some embodiments, the device labeling and assembly operations may be performed sequentially and automatically. For example, during the labeling process, a label with pre-printed artwork may be printed in-line with a lot number and expiration date; the label may be applied to the syringe. Components of the device, such as the finger flange and plunger rod, may be automatically assembled onto the syringe to form the device. Detection systems may monitor quality attributes and process parameters on syringes and assemblies, and syringes or devices not meeting pre-defined acceptance criteria may be automatically rejected and removed from the acceptable product flow-path. Acceptable quality limit (AQL) inspections for selected quality attributes may be performed throughout production and results recorded. Finished acceptable devices may be forward processed to blistering (e.g., packaging) and cart loading processes.

In some embodiments, process qualification runs may be executed to qualify the device labeling and assembly process, such as for an 8 mg prefilled syringe. As an illustrative example, process qualification runs may include approximately 25,000 to approximately 35,000 units. Process performance qualifications runs may be executed to validate the device labeling and assembly process for the 8 mg prefilled syringe. In some embodiments, both processes may be executed under normal operating procedures and nominal machine settings.

In some embodiments, the blistering packaging and cart loading unit operations may be performed sequentially and automatically. A blistering machine may create blister cavities from thermoplastic roll-stock, may automatically convey and load devices into the cavities, and may seal the cavities with a lid roll-stock with pre-printed artwork. In some embodiments, the lid may include a Tyvek® lid. The lid may be printed in-process with a lot number and expiration date of the device. In some embodiments according to the present disclosure, finished blister packs may be automatically conveyed into a loader machine.

The loader machine may remove empty racks from a sterilization cart and may load blister packs onto the cart according to a pre-defined pattern. Filled carts may be removed from the machine by operators, and replaced with empty carts for continuous operation until the batch is fully processed. In some embodiments, detection systems monitor quality attributes and process parameters on all blister packs, racks, and carts. Process outputs not meeting pre-defined acceptance criteria may be automatically rejected and removed from the acceptable product flow-path. Acceptable quality limit inspections for selected quality attributes may be performed throughout production and results recorded. In some embodiments according to the present disclosure, acceptable filled carts are forward processed to blister pack sterilization.

Similar to the device labeling and assembly process for an 8 mg syringe described above, the blistering (e.g. packaging) and cart loading steps may be evaluated. Performance qualification runs may be executed to validate the blistering and cart loading processes for the 8 mg syringe, including a low performance qualification, a high-performance qualification, and a nominal target run. Each performance qualification run may be performed for at least approximately one hour. Process performance qualification runs may be executed to validate the blistering and cart loading process for the 8 mg syringe.

In some embodiments, the VHP sterilization processes described herein may be validated. Chemical indicators (Cis) and biological indicators (BIs) may be placed on the carts of blister packaged prefilled syringes of the present disclosure in a pre-defined pattern; the carts may be loaded into the sterilization chamber shown and described herein in a pre-defined pattern. The sterilizing chambers may create a sterile environment within the blister packs using VHP. After sterilization, acceptable sterilized carts may be forward processed into the steps of sterilization cart unloading and final packaging.

In some embodiments, the validation of the VHP sterilization system and process may follow a step-wise approach. For example, the sterilization equipment and automated controls may have been previously qualified for another drug product presentation, according to an installation qualification or operational qualification protocol, to ensure the sterilization chamber and skidded equipment were installed properly and functioning as expected. The sterilization cycle may be validated, per a performance qualification protocol, to confirm the efficacy and repeatability of the developed cycle. The sterilization process may have been validated, according to the process performance qualification protocol, to ensure the robustness of the overall process, including manual processing steps, the sterilization process, and to perform extensive testing of the aflibercept drug product and packaging after exposure to VHP sterilization. The entire contents of the sealed packaging, including Tyvek®-sealed packaging, remain sterile until removal of the cover by the end user.

In some embodiments, at least one equipment validation cycle may be performed. A maximum load may be performed using one VHP asset and the minimum load may be performed using another VHP asset. Each run may use biological indicators and chemical indicators placed inside device blister packages to provide evidence that the desired sterilization had been achieved. In addition, a plurality (e.g., approximately 45) of samples of the sterility of the blister package may be taken from the load to provide further evidence of achievement of desired sterilization. For example, from approximately 40 to approximately 120 biological indicators may be used during the validation cycle, and from approximately 40 to approximately 120 chemical indicators may be used during the validation cycle.

In some embodiments, a process validation cycle may be performed to qualify the VHP critical process parameters. At least three maximum load cycle configuration runs and at least three minimum load cycle confirmation configuration runs may be performed. At least two temperature mapping activities may be performed to evaluate the production cycle. As described above, the process validation cycle may utilize biological indicators and/or chemical indicators placed inside the blister packages to indicate that desired sterilization have been achieved. Additional blister packages may be used the evaluate the sterility of packaging. Temperature loggers may be used to log the temperature distribution in the chamber.

As an example, approximately 72 to 144 biological indicators may be used during the process validation cycle, and approximately 8 to 24 chemical indicators may be used during the validation cycle. By way of further example, approximately 45 device packages may be evaluated with approximately 30 temperature loggers used to log the temperature distribution in the chamber during the process validation cycle.

In some embodiments, a process performance qualification cycle may be run to evaluate the sterilization process. For example, approximately five process performance qualification runs may be performed consisting of at least one maximum load, at least one minimum load, and at least approximately three minimum loads with double sterilization applied between two VHP sterilizers. The validation cycle may represent a cycle with a reduced VHP quantity and exposure time compared to a routine production cycle. The validation cycle may be used to ensure VHP distribution and penetration during process qualification cycles and all sterility challenge testing. The validation cycle may be defined as a cycle that achieves sterilization with no safety factor, which may be analogous to a half-cycle and/or fractional cycle approach used during sterilization process validation. A routine production cycle may use one additional sterilization pulse of VHP when compared to the validation cycle, which may provide a safety factor to ensure efficacy of the sterilization during routine processing. In further embodiments, during the temperature and aeration portion of the process qualification cycle, a greater number of VHP pressure pulses may be less desirable; a greater number of VHP pressure pulses may increase the risk of elevated temperatures and/or increased VHP in the sterilization chamber.

A variety of exemplary parameters may be evaluated during equipment and process validation cycles, including but not limited to: a number of VHP pulses, an amount of VHP injected per pulse (g), a total VHP injected per cycle (g), a vacuum level in the chamber (mbar), a post-injection hold time (min), a transition pressure (mbar), and/or a post-transition hold time (min).

In some embodiments, the devices may be configured to contain sterile aflibercept recombinant protein having a concentration of 114.3 mg/mL, in an aqueous buffered solution having a pH of approximately 5.8 and containing approximately 50 mM arginine monohydrochloride (e.g., L-arginine monohydrochloride), approximately 10 mM histidine (e.g., 3.65 mM L-histidine and 6.35 mM L-histidine monohydrochloride monohydrate), approximately 5% (w/v) sucrose, and approximately 0.03% (w/v) polysorbate 20, as described in detail herein. In some embodiments, the amount per batch (9.8 kg to 31.8 kg) may include approximately 1186 g to 3849 g of aflibercept; 109 g to 355 g of arginine monohydrochloride; 19.7 g to 83.9 g of histidine (e.g., 5.9 g to 19.1 g of L-histidine and 13.8 g to 44.8 g of L-histidine monohydrochloride monohydrate); 519 g to 1684 g of Sucrose; and 3.1 g to 10.1 g of Polysorbate 20. In further embodiments, the content per syringe may include approximately 8 mg of aflibercept; 0.737 mg of arginine monohydrochloride; 0.133 mg of histidine (e.g., 0.04 mg of L-histidine and 0.093 mg of L-histidine monohydrochloride monohydrate); 3.5 mg of Sucrose; and 0.021 mg of Polysorbate 20. In these embodiments, it should be appreciated that the dose delivery device contains an active ingredient of aflibercept, stabilizing agents of arginine monohydrochloride, Sucrose, and Polysorbate 20, and a buffering agent of histidine. An exemplary role of L-arginine monohydrochloride as a stabilizing agent in the drug product formulation may include reducing a rate of aggregation at the recommended storage condition. An exemplary role of L-histidine as a buffering agent in the drug product formulation may include maintaining a desired pH. An exemplary role of Sucrose as a thermal stabilizing agent and tonicity agent in the drug product formulation may include reducing a rate of aggregation. An exemplary role of Polysorbate 20 as a surfactant and interfacial stabilizing agent in the drug product formulation may include reducing a rate of aggregation and precipitation when the drug product is handled and agitated. The devices may be evaluated for one or more of their physical properties, identity, total protein contents, potency, isoaspartate assay, particulate matter, volume in container, maximum injection force, endotoxin content, sterility, purity, and/or change variants.

For example, the devices may be found to have the physical appearance of a liquid essentially free from visible particulates. The devices may have a clarity of less than or equal to III. The devices may have a color of less than or equal to BY5. The pH of the contents of the device may have a pH of approximately 5.9 and may conform to dot blot tests. The total protein contents, measured in absorbance at 280 nm, may be in the range from approximately 113.0 mg/mL to approximately 113.6 mg/mL.

In some embodiments, devices according to the present disclosure may have a potency in the range from approximately 94% to 113%. The isoaspartate content in mol isoaspartate/mol aflibercept may be in the range from approximately 0.06 to 0.08. The particulate matter of the contents of the devices may be evaluated based on particles/mL, including particles at least equal to or greater than 10 μm, at least equal to or greater than 25 μm, and/or at least equal to or greater than 50 μm. In some embodiments, the contents of the devices may have a quantity of particles at least equal to or greater than 10 μm in the range from approximately 1 to 8; a quantity of particles at least equal to or greater than 25 am in the range from approximately 0 to 2; and/or a quantity of particles at least equal to or greater than 50 μm in the range from approximately 0 to 1. In some embodiments, the volume of the contents in the syringe barrel may be in the range from approximately 0.11 mL to 0.17 mL.

In some embodiments, the present disclosure relate to systems and methods for the use of vaporized chemicals in sterilization processes, such as processes for sterilizing medical products. For example, embodiments of the present disclosure may relate to systems and methods for the terminal sterilization of medical products using vaporized hydrogen peroxide (VHP). More particularly, embodiments of the present disclosure may relate to, e.g., systems and methods for the terminal sterilization of medical products, such as pre-filled syringes (PFS).

It is generally desired that exposure to sterilization cycles be effective while having no adverse impact and minimized risk of damage or alteration to the load being sterilized. Medical products that undergo terminal sterilization, such as PFS, may thus require sterilization equipment, machinery, controls, cycle, and methods to address certain constraints and requirements in order to achieve appropriate sterilization and/or avoid damage to the medical products and/or devices, formulated drug substances, drug products, or other products. Such constraints and requirements may include, e.g.:
Medical products may be located within different parts of a sterilization chamber (e.g., quadrants or zones), which may, during a sterilization cycle, exhibit conditions that may differ from those in other parts of the chamber, such as temperature, pressure, water vapor concentration, humidity, or sterilant concentration. Such differing conditions may affect sterilization efficacy. Maintenance of a consistent environment throughout a sterilization chamber may be beneficial to ensuring sterilization efficacy is adequate as to all parts of a load.
An environment within a sterilization chamber may change during a sterilization cycle, in a way that affects the movement, state, or efficacy of sterilant and/or fluids in the chamber. For example, as sterilant is added to a chamber, a pressure within the chamber may increase. The pressure within the chamber may affect the ratio of condensed sterilant to vaporized sterilant. Changes in temperature, humidity, or other environmental characteristics may also affect how sterilant within the chamber and additional sterilant added to the chamber behaves. Sterilization systems and methods that adapt to changes in the environment or climate within the sterilization chamber during a sterilization phase, to maximize efficacy of sterilization, may be beneficial. Systems and methods that adapt to changes in the environment or climate of an area to maximize aeration and drying of that area may also be beneficial.
Medical products may be densely packed. For example, bulk packaged medical products may contain a large sum of fully assembled, packaged, and labeled medical products. In the case of terminal sterilization, sterilizing agents may need to traverse several layers of packaging materials, container materials, and/or labels, to effectively sterilize all aspects of a load, and to be appropriately removed from all aspects of the load. In some cases, packaging may include semi-permeable materials, which select for sterilizing agents in a particular phase (e.g., vapor).
In the case of some types of sterilization, such as terminal sterilization, sterilizing agents may need to traverse a semi-permeable membrane, either by heat or by mass, to sterilize the exterior of each medical product as well as the interior of packaging elements. Sterilizing agents may also need to be successfully removed through, e.g., a semi-permeable membrane, to avoid remaining as residue on a medical product. Traversal of a semi-permeable membrane may only be possible for sterilizing agent in a particular form, such as a vapor or gas.
Packaging for medical products may resist penetration of sterilization materials, and/or may be sensitive to temperature and pressure changes caused by sterilization. For example, a syringe may be packaged in a plastic ‘blister’ configured to house the syringe and restrict it from movement. Such a blister may be only somewhat permeable to sterilization materials, and/or may be sensitive to changes in pressure.

Using a combination of a vaporized chemical sterilant (e.g., VHP) and vaporized water in an environment in which temperature and pressure may be precisely controlled may allow for specific management of the environment to maximize the sterilant's contact with a sterilization load during a sterilization phase, and/or to maximize removal of the sterilant from a load during one or more subsequent aeration or drying phases. Some embodiments of the present disclosure are related to precisely controlling temperature, pressure, humidity, exposure times, and other environmental conditions. Environmental conditions may be adjusted in any portion of the sterilizing apparatus, before, during, and/or after a sterilization process is performed with the apparatus. For example, the environment of one or more portions of the apparatus where sterilant is introduced or removed, may be maintained or controlled to be within pre-determined conditions. As such, embodiments of the present disclosure may aid in improving the introduction and/or removal of a chemical sterilant in a sterilization apparatus (e.g., between the sterilization apparatus and an exterior of the apparatus, or between portions of the apparatus). Some embodiments of the present disclosure may be used in combination with disclosures of WIPO Publication No. WO 2018/182929, filed Mar. 6, 2018, which is herein incorporated by reference in its entirety. In some embodiments, sterilization of the devices described herein may undergo a cart normalization process for a period of approximately 1 hour as described in further detail herein. During sterilization, the devices may undergo a biological indicator normalization process for a period of approximately 1 hour as described in further detail herein.

Several characteristics of a vaporized chemical sterilant may (positively or negatively) affect the safety, efficacy, efficiency, and other aspects of sterilization processes for medical products. For example:
Chemical sterilant vapors and water vapors in an environment may adsorb to, and/or condense on, surfaces having relatively cooler temperatures within the environment. For example, during vapor sterilization of PFS loads, “cold spots” created by aqueous, high heat capacity, liquid product in each PFS, may serve to attract vapor adsorption and promote surface condensation. Moreover, changing the temperature of an environment (e.g., heating a sterilization chamber), may generate areas of relative warmth and coolness within the environment, which may in turn affect the relative temperature of a load in an area of relative warmth or coolness. For example, heating a sterilization chamber using a temperature control jacket may result in areas of the chamber closest to the jacket (e.g., a periphery of the chamber) becoming warmer than areas farther from the jacket (e.g., a middle of the chamber). The ambient heat in warmer areas may cause parts of a sterilization load in those areas to become relatively warmer as well. Chemical sterilant vapors and water vapors may preferentially adsorb to the surfaces of areas having relatively cooler temperatures as compared to the rest of the environment (“cold spots”); thus, vaporized chemical sterilant (e.g., VHP) may not distribute evenly between areas of relative warmth and coolness. While cooler areas may be subject to more thorough exposure to sterilant, warmer areas may experience more thorough aeration and drying.
VHP may preferentially adsorb onto surfaces as compared to water vapor, due to the fact that hydrogen peroxide is more dense and less volatile than water. In some instances, hydrogen peroxide and water vapor may be adsorbing and condensing on surfaces at the same time, with hydrogen peroxide adsorbing and condensing in greater quantities and percentages as compared to the water vapor, and in closer proximity to the surfaces of the sterilization load than the water vapor.
Multiple layers of adsorption may form on a single surface in a sterilization environment. In some instances, each layer of adsorption and/or condensation further away from the surface may contain less hydrogen peroxide and more water vapor, such that a gradient of hydrogen peroxide to water is formed on the surface. Hydrogen peroxide may preferentially adsorb and condense closer to the surface than water because of the thermodynamic behavior of binary mixtures of VHP and water vapor near or at saturation (e.g., a binary mixture of hydrogen peroxide and water at vapor/liquid equilibrium). Vapor/liquid equilibrium may be analogous to gas/adsorbate equilibrium for binary mixtures of VHP and water vapor in sterilization applications.
In some instances, condensed or adsorbed hydrogen peroxide may be difficult to remove from surfaces. For example, condensation of water vapor over the condensed/adsorbed hydrogen peroxide, or adsorption of water around the condensed/adsorbed hydrogen peroxide, may trap the hydrogen peroxide on the sterilized surface, or otherwise inhibit the removal of the hydrogen peroxide.
Differences in pressure throughout an environment, such as a sterilization chamber, may also affect efficacy of vaporized chemical sterilant. For example, sterilization efficacy may be greater at compressed air injection points of a sterilization chamber, compared to the rest of the chamber. Without being limited by theory, this may be due to the nature of gas within the chamber at a partial vacuum. Areas local to a compressed air injection point may experience a pressure wave or pulse having greater magnitude than areas farther from a compressed air injection point while the chamber is saturated with chemical sterilant. The pressure wave may cause greater condensation of chemical sterilant in areas local to compressed air injection points.
In some cases where it is desired that a sterilant traverse a semi-permeable membrane of a load to sterilize an interior area or volume covered by the membrane, a delay in migration of at least a portion of sterilant through the load has been observed. For example, in sterilization loads including a semi-permeable Tyvek® membrane, the hydrogen peroxide concentration within the membrane lagged, or was slower to equilibrate with, the hydrogen peroxide concentration outside of the membrane. No such delay or lag was observed with respect to water concentration. Thus, the relative strength of a sterilant may be lower for loads or portions of a load within a semi-permeable membrane (either for part or for all of a sterilization cycle) as compared to the strength of the sterilant outside of the membrane.
The speed with which pressure increases during introduction of vaporized sterilant into a sterilization chamber may negatively affect sterilization efficacy. A pressure increase to some degree may benefit introduction of sterilant to a load, promoting adsorption of the sterilant to the load. However, excessive pressure increases when an environment is at or near a level of VHP saturation, for example, may result in aggressive condensation of the VHP, which may compromise sterilization efficacy or subsequent aeration or drying efficacy. Allowing the pressure of an environment to be held at a level where vaporized sterilant may condense over time may result in over-condensation of vaporized sterilant. Conversely, decreasing the pressure of an environment after introducing vaporized sterilant (e.g., VHP) may allow more sterilant to remain in a vapor phase, which may improve the sterilant's efficacy in migrating through semi-permeable membranes and effecting sterilization of the interior(s) of a sterilization load.
During a sterilization phase (e.g., a sterilization pulse) increases in pressure may be faster than decreases in pressure (e.g., the rate of pressure increase during a sterilization pulse may be 150 millibar/minute faster than the rate of pressure decrease during the same pulse). This may promote travel of the sterilant (e.g., promote travel of sterilant through one or more layers of packaging). During aeration, the reverse may be employed to promote travel of sterilant from within layers of packaging to exterior of the packaging, and through the exhaust of the sterilization apparatus. For example, the rate of pressure decrease during an aeration pulse may be 150 millibar faster than the rate of pressure increase during the same pulse. Increased chamber temperatures may also increase the efficiency of aeration.
The saturation level of the sterilization chamber may also factor into the rate or direction of pressure adjustment. For example, pressure increases near atmospheric pressure should be avoided while the sterilization chamber is near saturation. For example, greater pressure changes may be used a lower sterilant concentrations, while large pressure changes at high sterilant concentrations may cause excess condensation, decreasing the efficiency of the sterilization.
Sterilizing agents may also need to be successfully removed from the load to avoid remaining as residue on or in a medical product. For example, in embodiments utilizing packaging including a semi-permeable membrane, traversal of the semi-permeable membrane may only be possible for sterilizing agent in a particular form, such as a vapor or gas. In some cases, stimulating mechanical movement (e.g., rocking, rotation, agitation, etc.) of some or all of a load may dislodge sterilant molecules adhered to a load, and may promote aeration and removal of a sterilant from the load. Low frequency pressure waves or acoustics generated within the sterilization chamber (for example, via a diaphragm or piston located within the chamber) may dislodge sterilant adhered to the load.
Immediately increasing the pressure following a sterilization pulse can cause unnecessary condensation and result in less efficacious aeration. Prior to aeration, the humidity of the sterilization chamber may be reduced to prevent excessive condensation. For example, the contents of the closed system may be passed through a condenser (e.g., a desiccant wheel) to reduce the humidity of the environment. In addition or alternatively, dry air may be injected, prior to, or during exhaustion, to reduce the overall humidity of the system.

Systems and methods disclosed herein may advantageously be used in improving the efficacy of sterilization, aeration, and/or drying cycles involving vaporized chemical sterilants. For example, systems and methods disclosed herein may provide for full (e.g., 100%) sterilization of medical products using VHP, followed by full (e.g., 100%) removal of VHP from sterilized products. Systems and methods disclosed herein may, e.g., increase efficiency, safety, and efficacy of sterilization, and/or decrease sterilization cycle time. While aspects of the present disclosure may be described with respect to the use of VHP in terminal sterilization of PFS, the present disclosure contemplates using the techniques and systems herein for movement of VHP and other chemical sterilants in other contexts as well (e.g., sterilization of other products, cleaning areas, addition/removal of vaporized chemical to any environment, etc.).

The present disclosure also contemplates performance of “moist chemical sterilization,” by which chemical sterilization may be achieved in the presence of water vapor. Comparison of “moist chemical sterilization” to “chemical sterilization” may be analogous, in some cases, to comparison of “moist heat sterilization” to “heat sterilization.” In some instances, moist chemical sterilization may be a more effective and efficient means of achieving sterilization than chemical sterilization technology that currently exists, in the same way that “moist heat sterilization” is considered to be, in some cases, more effective and efficient than only “heat sterilization.”

“Moist chemical sterilization” may take place when environmental conditions of relatively high chemical concentration, high water vapor concentration, and high pressure (e.g., above 400 millibar) act in concert to force the chemical and water vapor to behave as a binary mixture. In order to achieve the desired relatively high chemical concentration, high water vapor concentration, and high pressure, the area to be sterilized may be saturated with a combination of water vapor and sterilizing chemical (e.g., VHP), forcing vapor to condense on surfaces of the load. Most commercially available hydrogen peroxide is available and sold as aqueous liquid mixtures in varying concentrations (e.g., 3%, 15%, 35%, 59%), and thus, vaporizing hydrogen peroxide will generally simultaneously include vaporizing water.

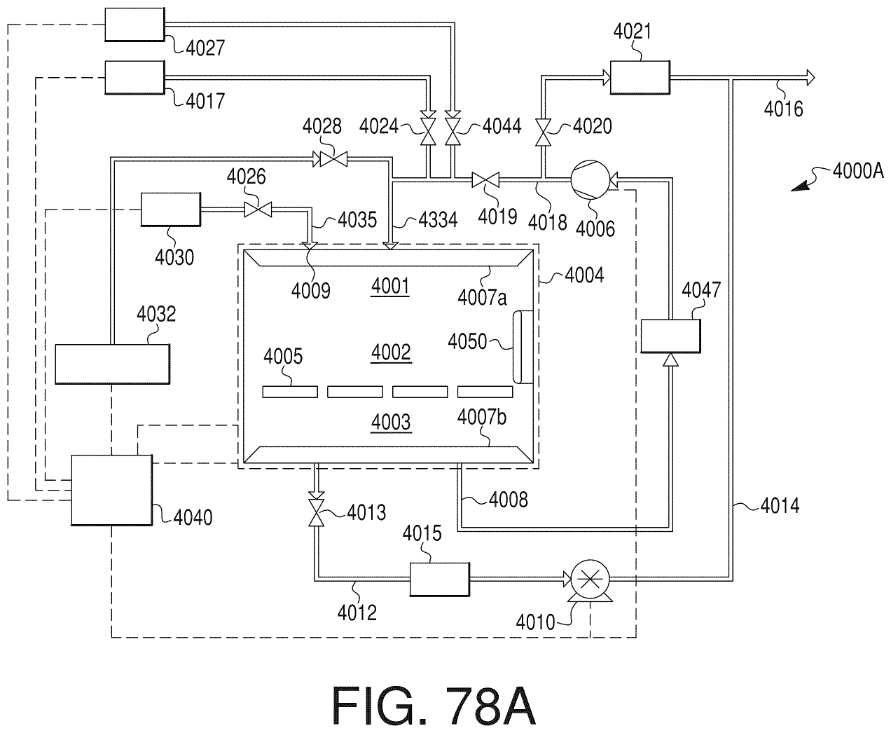

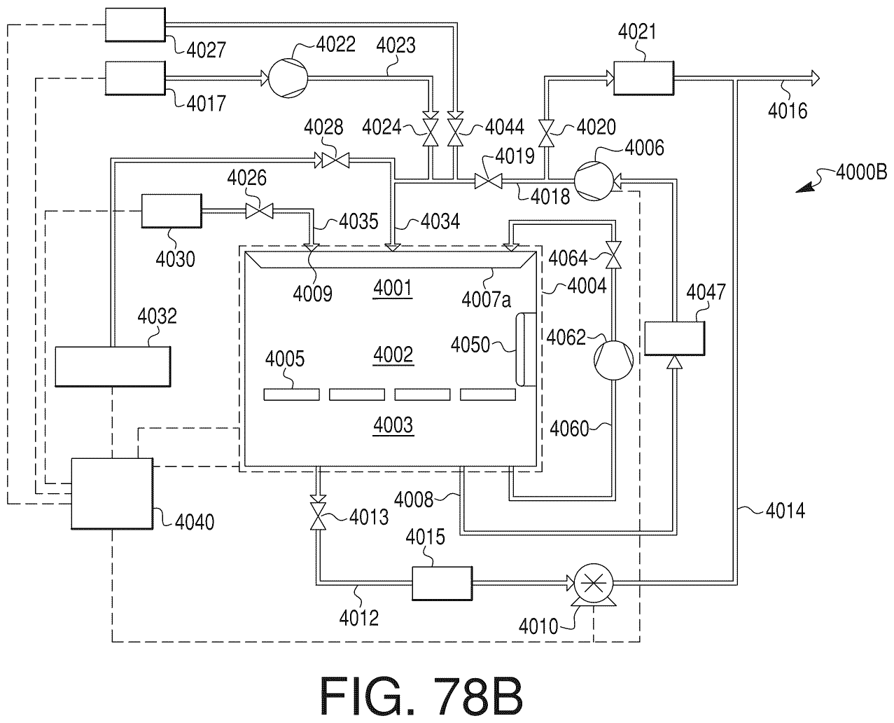

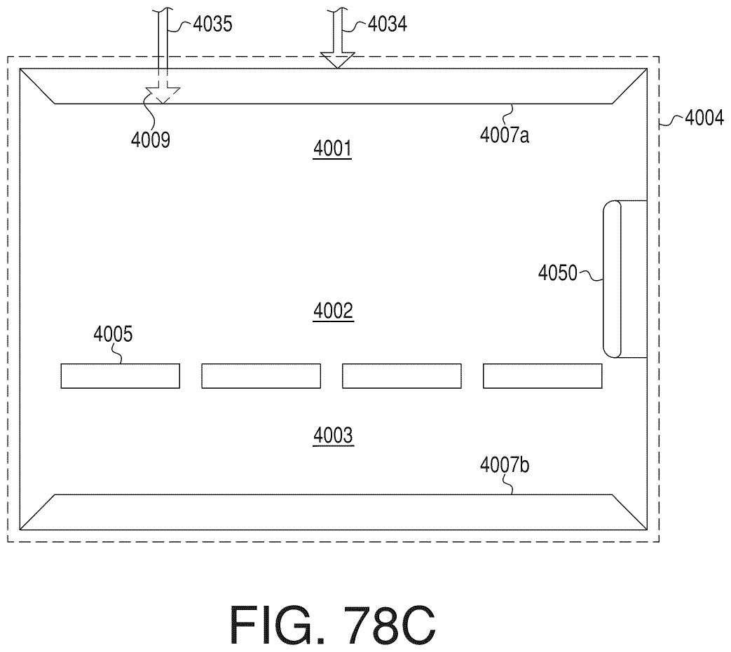

FIG. 78A depicts in schematic form an exemplary sterilization system 4000A for use in methods of the present disclosure, such as to sterilize one or more delivery devices (e.g., delivery device 2800). It is to be understood that sterilization system 4000A is merely exemplary, and methods disclosed herein may be used in many other systems, environments, and/or parts thereof. Sterilization system 4000A includes a sterilization chamber 4002, surrounded by a temperature control jacket 4004. Sterilization chamber 4002 has an interior cavity, including an upper interior 4001 and a lower interior 4003. Sterilization chamber 4002 is configured to house a sterilization load (e.g., a load including one or more products 4005) for sterilization. Although not shown, it should be appreciated that sterilization chamber 4002 may include one or more door rollers, solenoid valves, and various other movable components therein for facilitation sterilization of products 4005. Additionally and/or alternatively, sterilization chamber 4002 may include one or more biological indicators positioned therein for measuring one or more metrics relating to the VHP exposed therein, as described further below. An inlet conduit 4034, fluidly connected to sterilization chamber 4002, is configured to allow various fluids to enter sterilization chamber 4002. The inlet conduit 4034 may be connected to one or more distribution manifolds (e.g., diffusion plates, spray balls, or other structure configured to distribute a gas throughout the chamber).

In the examples shown in FIGS. 78A and 78C, a first distribution manifold 4007a is located near upper interior 4001 and a second distribution manifold 4007b is located near lower interior 4003. Placement of distribution manifolds 4007a, 4007b on opposing sides of sterilization chamber 4002 may promote even distribution of sterilant, air, or other introduced material.

Inlet conduit 4034 may connect to both first distribution manifold 4007a and second distribution manifold 4007b. In some embodiments, second distribution manifold 4007b is connected to a different inlet conduit than first distribution manifold 4007a. A second inlet conduit 4035 is also fluidly connected to sterilization chamber 4002, also to allow fluids to enter sterilization chamber 4002 via an inlet 4009. For example, ambient air from auxiliary ambient air supply 4030 may be introduced into sterilization chamber 4002 via inlet 4009.

A blower 4006 is fluidly connected to sterilization chamber 4002 via a blower exit conduit 4008. A blower circulation conduit 4018 fluidly connects blower 4006 to move fluids from blower exit conduit 4008 either towards an exhaust 4016, or back towards sterilization chamber 4002 via inlet conduit 4034. In some embodiments, blow exit conduit 4008 may include, or be coupled to, a condenser 4047. Condenser 4047 may include a desiccate wheel or other structure configured to remove water from the fluid passing through blower exit conduit 4008. An exhaust valve 4020 is located between blower circulation conduit 4018 and exhaust 4016, and selectively closes or opens a connection between blower circulation conduit 4018 and exhaust 4016. A recirculation valve 4019 is located between blower circulation conduit 4018 and inlet conduit 4034, and selectively closes or opens a connection between blower 4006 (e.g., via blower circulation conduit 4018) and inlet conduit 4034.

Still referring to FIG. 78A, blower 4006 may circulate air at a rate greater than 500 cubic feet per minute (cfm). In some embodiments, the rate of air circulation maintained by blower 4006 may be less than 1000 cfm. If blower 4006 is moving too much fluid, sterilant will be displaced near distribution manifolds 4007a, 4007b, decreasing the sterilization efficiency. A vacuum pump 4010 may be fluidly connected to sterilization chamber 4002, via a vacuum conduit 4012. Vacuum conduit 4012 may include, or be connected to, a catalytic converter 4015. A vacuum valve 4013 located between sterilization chamber 4002 and vacuum conduit 4012, may selectively allow, partially allows, or block, flow from sterilization chamber 4002 (e.g., through catalytic converter 4015) to vacuum pump 4010. A vacuum exhaust conduit 4014 fluidly connects vacuum pump 4010 to exhaust 4016.

Sterilization system 4000A may include several supplies of air and/or vapor from which fluid may be introduced into sterilization chamber 4002 via inlet conduit 4034 or inlet conduit 4035. A dry air supply 4027 may be configured to supply dry (makeup) air to sterilization chamber 4002 via inlet conduit 4034. In some embodiments, dry air supply 4027 is compressed dry air. A dry air valve 4044 may be coupled to the fluid connection between dry air supply 4027 and inlet conduit 4034. The dry air valve 4044 may selectively allow, partially allow, or block, flow of dry air from dry air supply 4027 to sterilization chamber 4002 via inlet conduit 4034. An ambient air supply 4017 may be configured to supply ambient air (e.g., air comprising more humidity than dry air from dry air supply 4027) to sterilization chamber 4002, via inlet conduit 4034. In some embodiments, ambient air may be drawn from an environment (e.g., room) around the sterilization system 4000A. An ambient air valve 4024 may be coupled to the fluid connection between ambient air supply 4017 and inlet conduit 4034. The ambient air valve 4024 may selectively allow, partially allow, or block, flow of ambient air from ambient air supply 4017 to sterilization chamber 4002 via inlet conduit 4034.

Still referring to FIG. 78A, ambient air supply 4017, for example, may be any supply of air (e.g., room air, or compressed air) or other fluid external from the rest of sterilization system 4000A. In some embodiments, ambient air supply 4017 may be a supply of “room air” surrounding sterilization system 4000A, which may have gone through an indoor filtration system. In some embodiments, ambient air supply 4017 may include more water vapor than “room air.” In some embodiments, ambient air supply 4017 may be a supply of filtered outdoor air.

A VHP injector 4032, fluidly connected to inlet conduit 4034, is configured to inject VHP to sterilization chamber 4002 via inlet conduit 4034. A VHP injector valve 4028 is coupled to the fluid connection between VHP injector 4032 and inlet conduit 4034, and selectively allows, partially allows, or blocks flow of VHP from VHP injector 4032 to sterilization chamber 4002 via inlet conduit 4034. VHP injector 4032 may include a supply of VHP, or VHP and vaporized water, and may be configured to inject VHP or a combination of VHP and vaporized water into sterilization chamber 4002 via, e.g., inlet conduit 4034. VHP injector 4032 may be configured to inject vapor into sterilization chamber 4002 (or inlet conduit 4034) at an adjustable concentration. Although not shown, sterilization system 4000A may include a restriction mechanism upstream from VHP injector 4032, and the restriction mechanism may be configured to restrict a flow path between the supply of liquid HP and VHP injector 4032. In some embodiments, the restriction mechanism may include a variable orifice flow meter. Additionally and/or alternatively, inlet conduit 4034 may include a temperature-controlled heat trace that is configured to heat the VHP delivered from VHP injector 4032 to sterilization chamber 4002.

Still referring to FIG. 78A, depending on the position of dry air valve 4044, ambient air valve 4024, and VHP injector valve 4028: dry air from dry air supply 4027, ambient air from ambient air supply 4017, VHP from VHP injector 4032, or a combination thereof, may enter sterilization chamber 4002 via inlet conduit 4034. For example, during pre-conditioning, ambient air valve 4024 may be in a position such that ambient air from ambient air supply 4017 is blocked from entering inlet conduit 4034, VHP injector valve 4028 may be in a position such that VHP from VHP injector 4032 is blocked from entering inlet conduit 4034, and dry air valve 4044 may be in a position such that dry air flows from dry air supply 4027 to sterilization chamber 4002, via inlet conduit 4034. In such a configuration, only dry air flows to the sterilization chamber 4002, allowing for faster pre-conditioning.

In another configuration, ambient air valve 4024 may be in a position such that ambient air flows from ambient air supply 4017 to sterilization chamber 4002 (i.e., via inlet conduit 4034), VHP injector valve 4028 may be in a position such that VHP flows from VHP injector 4032 to sterilization chamber 4002 (i.e., via inlet conduit 4034), and dry air valve 4044 may be in a position such that dry air from dry air supply 4027 is blocked from entering inlet conduit 4034. In such a configuration, ambient air may function as a binding media to carry VHP into sterilization chamber. Dry air or a combination of dry air and ambient air may also be used as binding media. Ambient air may be more effective as binding media than dry air. Humidity (e.g., water) from the ambient air used as binding media may be removed from sterilization chamber 4002 (e.g., via condenser 4047) after VHP has been introduced.

An auxiliary ambient air supply 4030 fluidly connected to inlet conduit 4035 may be configured to supply ambient air to sterilization chamber 4002 via inlet conduit 4035. An auxiliary supply valve 4026 is coupled to the fluid connection between auxiliary ambient air supply 4030 and inlet conduit 4035, and is configured to selectively allow, partially allow, or block flow of ambient air from auxiliary ambient air supply 4030 to sterilization chamber 4002 via inlet conduit 4035.

Dry air supply 4027 and auxiliary ambient air supply 4030 may have the same composition or different compositions. Dry air supply 4027 may be a supply of air having a relatively low humidity, such that it may be used to dry sterilization chamber 4002 (e.g., a portion of sterilization chamber 4002) and/or one or more of blower exit conduit 4008, vacuum conduit 4012, vacuum exhaust conduit 4014, blower circulation conduit 4018, and inlet conduit. For example, in some embodiments, air in dry air supply 4027 may include a dew point of, e.g., −10 degrees Celsius or less, −40 degrees Celsius or less, or anywhere between −10 degrees Celsius and −40 degrees Celsius. In some embodiments, dry air supply 4027 may be a supply of hygienic dry air, such as air that has been sterilized or otherwise filtered to at least 0.2 microns. In some embodiments, VHP injector 4032 may be configured to vaporize the sterilant (e.g., VHP) and heat the filtered air. In some embodiments, either or both ambient air supplies 4027, 4030 may be a sealed supply of air. In some embodiments, dry air supply 4027 may be a supply of compressed air. As described herein, conditioning gas, distribution gas, and/or aeration gas may be provided from dry air supply 4127, ambient air supply 4017, and/or auxiliary ambient air supply 4030.

Still referring to FIG. 78A, sterilization system 4000A may be configured to run sterilization cycles within sterilization chamber 4002 at a variety of temperatures and pressures, and for a variety of time durations and/or time intervals. In some embodiments, the temperature(s), pressure(s), and time interval(s) at which sterilization system 4000A may run sterilization cycles may be selectively and individually modified and customized. Moreover, temperature(s), pressure(s), and time intervals(s) may be adjusted during sterilization cycles, e.g., to effect improved distribution, migration, and/or removal of sterilants. For example, a pressure reaching about 500 mBar, a minimum temperature of about 26° C., a time interval (PV231 delay) of about 1500 msec, and/or a leak test including about 13 mBar hold time of about 5 minutes, may be one or more required parameters for commencement of the sterilization cycle.

Sterilization system 4000A may be configured to control the environment in the interior of sterilization chamber 4002, including temperature, pressure, humidity, atmosphere, intake of fluids, and exit of fluids. Mechanisms for controlling temperature include: temperature modulation of input fluid streams and/or recirculating fluid streams, temperature modulation of portions of the sterilization chamber itself, and temperature modulation of other components of the system 4000A (e.g., intake conduit 4034, blower exit conduit 4008, vacuum pump 4010, temperature control jacket 4004, blower circulation conduit 4018, blower 4006, recirculation valve 4019, ambient air supply 4017, dry air supply 4027, VHP injector 4032, ambient air valve 4024, dry air valve 4044, VHP injector valve 4028, distribution manifolds 4007a, 4007b).

For example, a Vaprox may include about 5 gallons at room temperature with a Vaprox reservoir maintained at room temperature in an injection cabinet. A minimum pressure may be about 480 mBar and a maximum pressure may be about an ambient and/or room pressure. In some embodiments, minimum pressure rise may be about 40 mBar and a maximum pressure rise may be about 440 mBar. A low temperature may be below about 21° C. and a maximum temperature may be about 35° C. A jacket set point temperature may be about 28° C.+/−7° C. A vaporizer set point temperature may be about 110° C.+/−15° C., while a preheater set point temperature may be about 75° C. In further examples, a chamber pressure low may be below about 485 mBar. It should be appreciated that the parameters of the sterilization system 4000A are merely exemplary such that various other suitable parameters may be incorporated into sterilization system 4000A without departing from a scope of this disclosure.

Still referring to FIG. 78A, sterilization chamber 4002 (e.g., a portion of sterilization chamber 4002, such as, upper interior 4001 or lower interior 4003) may include, or be fluidly connected to one or more pistons 4050 or diaphragm that may be actuated, inflated, deflated, or otherwise altered, to modulate the pressure of sterilization chamber 4002 without introduction or removal of matter. The one or more pistons 4050 or diaphragms may be configured to create pressure waves or generate low frequency pulses. These pressure waves and pulses may be used to affect (e.g., promote) condensation of sterilant on a surface of the load. Further, sterilization system 4000A may include any suitable number and location of sensors configured to sense, e.g., temperature, pressure, flow, chemical concentration, or other parameters throughout sterilization system 4000A, including in sterilization chamber 4002, temperature control jacket 4004, blower 4006, vacuum pump 4010, and/or any of conduits 4008, 4012, 4014, 4018, and 4034. Such sensors may be configured to transmit sensed data to, e.g., controller 4040 and/or a human-machine interface.

Sterilization chamber 4002 may be a sealable chamber defining an interior, including upper interior 4001 and lower interior 4003. Sterilization chamber 4002 may be openable into an open configuration, such that one or more items, e.g., products 4005, may be placed inside as a part of a load for sterilization, and may be removed subsequent to sterilization. In some embodiments, sterilization chamber 4002 may have an operating orientation, e.g., such that upper interior 4001 is located above lower interior 4003, and such that matter may fall (e.g., under the forces of gravity) from the vicinity of upper interior 4001 towards lower interior 4003. Sterilization chamber 4002 may have one or more delivery apparatuses to which one or more of inlet conduit 4034 and inlet conduit 4035 may be connected.

As depicted in FIG. 78A, for example, distribution manifolds 4007a, 4007b are two such delivery apparatuses. Distribution manifolds 4007a, 4007b may be configured to disperse gas, vapor, or liquid into sterilization chamber 4002 in a given configuration, such as a stream or an even spray across sterilization chamber 4002. For example, distribution manifold 4007a may distribute gas, vapor, or liquid across upper interior 4001 and distribution manifold 4007b may distribute gas, vapor, or liquid across lower interior 4003. Inlet 4009 is another such delivery apparatus. Inlet 4009 may also be configured to disperse gas, vapor, or liquid into sterilization chamber 4002 in a given configuration, such as a stream, or an even spray across upper interior 4001, or another portion of sterilization chamber 4002.

In some embodiments, a distribution manifold (e.g., distribution manifold 4007a) may be configured to disperse gas, vapor, or liquid into sterilization chamber 4002 in one configuration, such as an even spray, and inlet 4009 may be configured to disperse gas or vapor into sterilization chamber 4002 in a different configuration, such as in a stream. In some embodiments, there may be no inlet 4009, and both inlet conduits 4034 and 4035 may be connected to one or more distribution manifolds 4007a, 4007b. Temperature control jacket 4004 may be any material surrounding sterilization chamber 4002, that is configured or effective to afford temperature control to the environment inside sterilization chamber 4002. In some embodiments, for example, temperature control jacket 4004 may be a water jacket surrounding sterilization chamber 4002. In such embodiments, a temperature and/or a flow of water or other liquid through temperature control jacket 104 may be controlled by, e.g. controller 4040. In further embodiments, one or more doors on sterilization chamber 4002 may include a temperature-controlled heat trace that is configured to heat the environment inside sterilization chamber 4002.

Products 4005 may be any item or items in a load suitable for sterilization using sterilization system 4000A. In some embodiments, products 4005 may be medical products (e.g., delivery device 2800) in primary packaging (e.g., packaging 3000), secondary packaging, or both. In some embodiments, products 4005 may be medical products having moving parts or parts otherwise sensitive to deep vacuum environments, such as environments having pressure of less than about 100 millibars. In some embodiments, products 4005 may be, e.g., containers filled with a volume of formulated drug substance. For example, products 4005 may be vials or PFS. In some embodiments, products 4005 may include or may be covered by semi-permeable packaging, such as a semi-permeable membrane, through which vapor or gas may pass. In some embodiments, products 4005 may be packaged in a packaging (e.g., packaging 3000) having a length of approximately 140 mm+/−5% and/or ranging from approximately 135 mm to approximately 145 mm. For example, the length may be approximately 135 mm, 136 mm, 137 mm, 138 mm, 139 mm, 140 mm, 141 mm, 142 mm, 143 mm, 144 mm, and/or 148 mm. In some embodiments, products 4005 may be packaged in a packaging (e.g., packaging 3000) having a width range from approximately 70 mm to approximately 80 mm, such as approximately 70 mm, 71 mm, 72 mm, 73 mm, 74 mm, 75 mm, 76 mm, 77 mm, 78 mm, 79 mm, and/or 80 mm. In some embodiments, products 4005 may be packaged in a packaging (e.g., packaging 3000) having a height of approximately 26 mm+/−5% and/or ranging from approximately 20 mm to approximately 30 mm, such as approximately 20 mm, 21 mm, 22 mm, 23 mm, 24 mm, 25 mm, 26 mm, 27 mm, 28 mm, 29 mm, and/or 30 mm. In some embodiments, products 4005 may be packaged in a packaging (e.g., packaging 3000) having a polyethylene terephthalate glycol (PETG) film. In some embodiments, the PETG film packaging may have a thickness of approximately 0.508 mm+/−5%. In some embodiments, the PETG film may have a uniform thickness. In other embodiments, the PETG film may have a nonuniform thickness.

In further embodiments, products 4005 may be or include medical products sensitive to high temperatures, e.g., above 30° C. Such medical products may include, for example, formulated drug substances or other compositions that may be sensitive to high temperatures, such as proteins (e.g., antibodies or enzymes), fragments thereof, any antigen-binding molecules, nucleic acids, blood, blood components, vaccines, allergenics, gene therapy medicaments, tissues, other biologics, etc. For example, products 4005 may be packaged PFS containing a formulated drug substance that includes an antibody or an adeno-associated virus (AAV).

In some embodiments, products 4005 may include drug products including a large molecule, e.g., a molecular weight of 30 kDA or greater. In some embodiments, products 4005 may include ingredients such as, e.g., aflibercept, alirocumab, abicipar pegol, bevacizumab, brolucizumab, conbercept, dupilumab, evolocumab, tocilizumab, certolizumab, abatacept, rituximab, infliximab, ranibizumab, sarilumab, adalimumab, anakinra, trastuzumab, pegfilgrastim, interferon beta-1a, insulin glargine [rDNA origin], epoetin alpha, darbepoetin, filigrastim, golimumab, etanercept, antigen-binding fragments of any of the above, or combinations of such binding domains, such as a bispecific antibody to VEGF or angiopoietin-2, among others. It should be appreciated that aflibercept may include a purified anti-VEGF agent comprising a VEGF binding portion.

In some embodiments, products 4005 may include therapeutic products for ophthalmic diseases, including for the treatment of patients with Neovascular (Wet) Age-related Macular Degeneration (AMD), Macular Edema following Retinal Vein Occlusion (RVO), Diabetic Macular Edema (DME), and Diabetic Retinopathy (DR). In particular, large molecule and small molecule antagonists of VEGF and/or ANG-2, such as aflibercept, ranibizumab, bevacizumab, conbercept, OPT-302, RTH258 (brolucizumab), abicipar pegol (a pegylated designed ankyrin repeating protein (DARPin)), RG7716, or fragments thereof and in any concentration may be included in products 4005. In some embodiments, products 4005 may be products for cosmetic applications or medical dermatology, such as treatment or diagnosis of allergic responses.

Blower 4006 may be, for example, a blower having the capacity to forcibly draw vapor and gas from lower interior 4003 of sterilization chamber 4002 through blower exit conduit 4008, optionally through condenser 4047, and to reintroduce said vapor and gas back to upper interior 4001 of sterilization chamber 4002 via inlet conduit 4034 (or, alternatively, to draw such vapor and gas through exhaust valve 4020 and catalytic converter 4021, to exhaust 4016). In some embodiments, blower 4006 may be external to sterilization chamber 4002, as shown in FIG. 78A. In other embodiments, blower 4006 may be disposed within sterilization chamber 4002. In some embodiments, blower 4006 may be configured to draw vapor and gas from lower interior 4003 of sterilization chamber 4002 and reintroduce said vapor and gas back to upper interior 4001 with sufficient force to create a flow of vapor and gas from upper interior 4001 to lower interior 4003 of sterilization chamber 4002. This flow may be termed a “turbulent flow.” In some embodiments, the force with which blower 4006 may operate may be adjustable (via, for example, controller 4040), such that a more turbulent (e.g., more forceful), or less turbulent, flow of vapor and gas within sterilization chamber 4002 may be generated. In some embodiments, blower 4006 may be configured to generate a stronger force to draw vapor and gas than, e.g., vacuum pump 4010.

Vacuum pump 4010 may be a vacuum pump having the capacity to draw gas from the interior (e.g., lower interior 4003) of sterilization chamber 4002, via vacuum conduit 4012 and catalytic converter 4015, and towards exhaust 4016, thereby creating a vacuum within sterilization chamber 4002 and/or a closed system containing sterilization chamber 4002 and, e.g., blower 4006. Vacuum pump 4010 may be fluidly connected to exhaust 4016 via, e.g., vacuum exhaust conduit 4014. In some embodiments, exhausts from vacuum pump 4010 and blower 4006 may be separated instead of being combined into one. In some embodiments, vacuum-type functions may also or alternately be performed by, e.g., blower 4006, which may selectively circulate vapor and gas out of and into sterilization chamber 4002 or out of sterilization chamber 4002, through exhaust valve 4020, and towards exhaust 4016. Exhaust valve 4020 may be selectively opened or closed so as to permit or prevent flow of gas or vapor from blower circulation conduit 4018 towards exhaust 4016 or towards inlet conduit 4034 for reintroduction into sterilization chamber 4002. Exhaust valve 4020 may be manually controlled, or may be controlled by, e.g., controller 4040.

Still referring to FIG. 78A, catalytic converter 4015, catalytic converter 4021, or both may be, for example, any catalytic converters known in the art suitable for converting toxic gaseous or vaporized fluids circulated within sterilization system 4000A, e.g., during a sterilization cycle, to less toxic gases or vapors. For example, catalytic converters 4015, 4021 may be configured to convert VHP into water vapor, oxygen, and/or other non-toxic fluids. A controller 4040 is connected to one or more other components of sterilization system 4000A, such as sterilization chamber 4002, temperature control jacket 4004, blower 4006, VHP injector 4032, moist makeup air supply 4017, dry air supply 4027, auxiliary ambient air supply 4030, vacuum pump 4010, piston 4050, catalytic converters 4015, 4021, condenser 4047, distribution manifolds 4007, 4007b, conduits 4012, 4008, 4018, 4034, 4035, valves 4013, 4019, 4020, 4024, 4026, 4044, and/or any other components of sterilization system 4000A. Some or all aspects of sterilization system 4000A may be controllable by, e.g., controller 4040. For example, controller 4040 may be in communication with one or more valves 4013, 4019, 4020, 4024, 4026, 4044, and may be configured to control, adjust, and/or monitor the position of the one or more valves 4013, 4019, 4020, 4024, 4026, 4044. In addition or alternatively, the one or more valves 4013, 4019, 4020, 4024, 4026, 4044 may be manually operated.

Controller 4040 may be, for example, an analog or digital controller configured to alter aspects of the environment of sterilization chamber 4002 such as an internal temperature or pressure of sterilization chamber 4002 and/or one or more of blower 4006, vacuum pump 4010, ambient air supply 4017, dry air supply 127, auxiliary ambient air supply 4030, VHP injector 4032, exhaust 4016, one or more of valves 4013, 4019, 4020, 4024, 4026, and 4028, one or more of catalytic converters 4015, 4021, one or more of conduits 4008, 4012, 4014, 4016, 4018, and 4034, and any and/or other aspects of sterilization system 4000A. In some embodiments, sterilization system 4000A may be controllable by multiple controllers 4040. In other embodiments, sterilization system may only have one controller 4040. In some embodiments, controller 4040 may be a digital controller, such as a programmable logic controller.

In some embodiments, controller 4040 may be pre-programmed to execute one or more sterilization, aeration, drying, and/or cleaning cycles using sterilization system 4000A. In some embodiments, sterilization system 4000A may be controllable by a controller having one or more human machine interface (“HMI”) elements, which may be configured to allow a user to input or alter desired parameters for a cycle, which may be executable by a controller on or operably coupled to sterilization system 4000A. Thus, in some embodiments, HMI elements may be used to program a customized cycle for execution by sterilization system 4000A. For example, in some embodiments, sterilization system 4000A may be controllable by a controller connected to, e.g., a computer, tablet, or handheld device having a display. Such a display may include, for example, options to select or alter a desired temperature, pressure, time, amount of VHP intake, amount of dry air, compressed air, or room air intake, etc., for one or more steps of a cycle.

The controller 4040 may include one or more alarm system that stop a sterilization process or display an alert if an alarm condition is met. For example, if the sterilization chamber 4002 fails to pressurize during a leak test, the controller 4040 may stop the sterilization process. Other examples of alarm conditions that may trigger a stop of the sterilization process or displaying of an alert can include maximum or minimum temperature thresholds being exceeded, maximum or minimum pressure thresholds being exceeded, or a time of a step in the sterilization process taking longer than a pre-programmed threshold.

FIG. 78B depicts in schematic form an exemplary sterilization system 4000B for use in methods of the present disclosure. It is to be understood that sterilization system 4000B is merely exemplary, and methods disclosed herein may be used in many other systems, environments, and/or parts thereof. For example, components, aspects, and/or structures of sterilization system 4000A may be used in combination and/or as alternatives to components, aspects, and/or structures of sterilization system 4000B. Except for as described otherwise herein, components and aspects of sterilization system 4000B may be structurally similar and/or function similarly to the similarly labeled components and aspects of sterilization system 4000A.

Still referring to FIG. 78B, sterilization system 4000B may include a first distribution manifold 4007a within an upper interior 4001 of sterilization chamber 4002. Sterilization system 400B may further include a second distribution manifold (not pictured) also within upper interior 4001 of sterilization chamber 4002. In some embodiments, sterilization system 4000B does not include a second distribution manifold 4007b. Sterilization system 4000B may comprise a recirculation conduit 4060, a recirculation blower 4062, and a recirculation valve 4064. Recirculation blower 4062 may be fluidly connected to sterilization chamber 4002 via recirculation conduit 4060. Recirculation conduit 4060 may fluidly connect recirculation blower 4062 to move fluids out of sterilization chamber 4002 (e.g., at lower interior 4003), through recirculation conduit 4060, and back into sterilization chamber 4002 (e.g., at upper interior 4001). A recirculation valve 4064 may be located between recirculation blower 4062 and sterilization chamber 4002, and selectively closes or opens a connection between recirculation blower conduit 4060 and sterilization chamber 4002. Recirculation valve 4064 may be positioned such that it is located between recirculation blower 4062 and upper interior 4001 of sterilization chamber 4002, as shown in FIG. 78B. Alternatively, recirculation valve 4064 may be positioned such that it is located between recirculation blower 4062 and lower interior 4003 of sterilization chamber 4002.

Recirculation blower 4062 may circulate air at a rate greater than 500 cubic feet per minute (cfm). In some embodiments, the rate of air circulation maintained by recirculation blower 4062 may be less than 1000 cfm. If recirculation blower 4062 is moving too much fluid, sterilant will be displaced near distribution manifold 4007a, decreasing the sterilization efficiency.

Recirculation blower 4062 may be, for example, a blower having the capacity to forcibly draw vapor and gas from lower interior 4003 of sterilization chamber 4002 through recirculation conduit 4060, and to reintroduce said vapor and gas back to upper interior 4001 of sterilization chamber 4002. In some embodiments, recirculation blower 4062 may be external to sterilization chamber 4002, as shown in FIG. 78B. In other embodiments, recirculation blower 4062 may be disposed within sterilization chamber 4002. In some embodiments, recirculation blower 4062 may be configured to draw vapor and gas from lower interior 4003 of sterilization chamber 4002 and reintroduce said vapor and gas back to upper interior 4001 with sufficient force to create a flow of vapor and gas from upper interior 4001 to lower interior 4003 of sterilization chamber 4002. This flow may be termed a “turbulent flow.” In some embodiments, the force with which recirculation blower 4062 may operate may be adjustable (via, for example, controller 4040), such that a more turbulent (e.g., more forceful), or less turbulent, flow of vapor and gas within sterilization chamber 4002 may be generated. In some embodiments, recirculation blower 4062 may be configured to generate a stronger force to draw vapor and gas than, e.g., vacuum pump 4010.

Sterilization system 4000B may include an ambient air supply 4017, a ambient air supply blower 4022, and ambient air supply conduit 4023. As shown in FIG. 78B, makeup air supply conduit 4023 may extend from ambient air supply 4017 to ambient air valve 4024 with ambient air supply blower 4022 disposed between ambient air supply 4017 and ambient air valve 4024.

In sterilization system 4000B, pressure change rates within sterilization chamber 4002 may be controlled by controlling fluid flow rates through ambient air supply conduit 4023 and inlet conduit 4035, for example by controlling a relative flow rate between ambient air supply conduit 4023 and inlet conduit 4035. Controlling the relative flow rate between ambient air supply conduit 4023 and inlet conduit 4035 can be achieved by selecting an appropriately sized conduit such that a rate of pressure change by fluid introduced via ambient air supply conduit 4023 is different than a rate of pressure change by fluid introduced via inlet conduit 4035. For example, ambient air supply conduit 4023 may have an inner diameter which is greater than the inner diameter of inlet conduit 4035, such that a rate of pressure change by fluid introduced via ambient air supply conduit 4023 is greater than a rate of pressure change by fluid introduce via inlet conduit 4035.

In some aspects, a ratio of an inner diameter of ambient air supply conduit 4023 to an inner diameter of inlet conduit 4035 may be selected to provide a desired difference in flowrates, and thereby pressure change rates between fluid introduced via ambient air supply conduit 4023 relative to fluid introduced via inlet conduit 4035. For example, a ratio of an inner diameter of ambient air supply conduit 4023 to an inner diameter of inlet conduit 4035 may be about 15:1 to about 5:1, such as, for example, about 11:1 to about 7:1, about 15:1, about 14:1, about 13:1 about 12:1, about 11:1, about 10:1, about 9.7:1, about 9.5:1, about 9.3:1, about 9:1, about 8.7:1, about 8.5:1, about 8.3:1, about 8:1, about 7.7:1, about 7.5:1, about 7.3:1, about 7:1, about 6.7:1, about 6.5:1, about 6.3:1, about 6:1, about 5.7:1, about 5.5:1, about 5.3:1, or about 5:1.

FIG. 78C depicts an expanded view of sterilization chamber 4002. Sterilization chamber 4002 may serve as an exemplary environment in which many aspects of the present disclosure may be applicable. It is to be understood, however, that sterilization chamber 4002 is merely exemplary, and that aspects of the present disclosure may be applicable in many other environments. Variations in temperature, pressure, humidity, and air/fluid flow may characterize different portions of sterilization chamber 4002. For example, temperature control jacket 4004 may be configured to control a temperature within sterilization chamber 4002, but may have a more immediate effect on a periphery of sterilization chamber 4002 than on a more central part of its interior. As has been described previously, temperature control jacket 4004 may more immediately affect the temperature of products 4005 (i.e., a load) closer to the periphery of sterilization chamber 4002 than to the middle of sterilization chamber 4002, causing products 4005 closer to the periphery to be, e.g., warmer than products 4005 in the middle.

As another example, distribution manifolds 4007a, 4007b and/or inlet 4009 may have surface temperatures differing from an average interior temperature of sterilization chamber 4002 (e.g., they may be cooler or warmer than an average interior temperature of sterilization chamber 4002). Additionally, during operation of sterilization system 4000A and/or sterilization system 4000B, distribution manifolds 4007a, 4007b, inlet 4009, a diaphragm (not pictured), and/or piston 4050 may create localized areas of relatively higher pressure around themselves as they inject fluid into sterilization chamber 4002 or create a pressure wave. This may cause greater condensation of fluids in areas closer to the source of the pressure wave as compared to areas farther away. For example, sterilant dispersed from one of the distribution manifolds 4007a, 4007b may be more likely to condense on products 4005 located closer to distribution manifold 4007a, 4007b than products 4005 that are farther away.

As has been described previously, products 4005 may include one or more semi-permeable membranes through which vapor or gas may flow, such as a cover on packaging for a medical device or drug product. Semi-permeable membranes may, in some cases, not allow liquid to pass through them. In some embodiments, sterilization of areas on both sides of the semi-permeable membrane is desired.

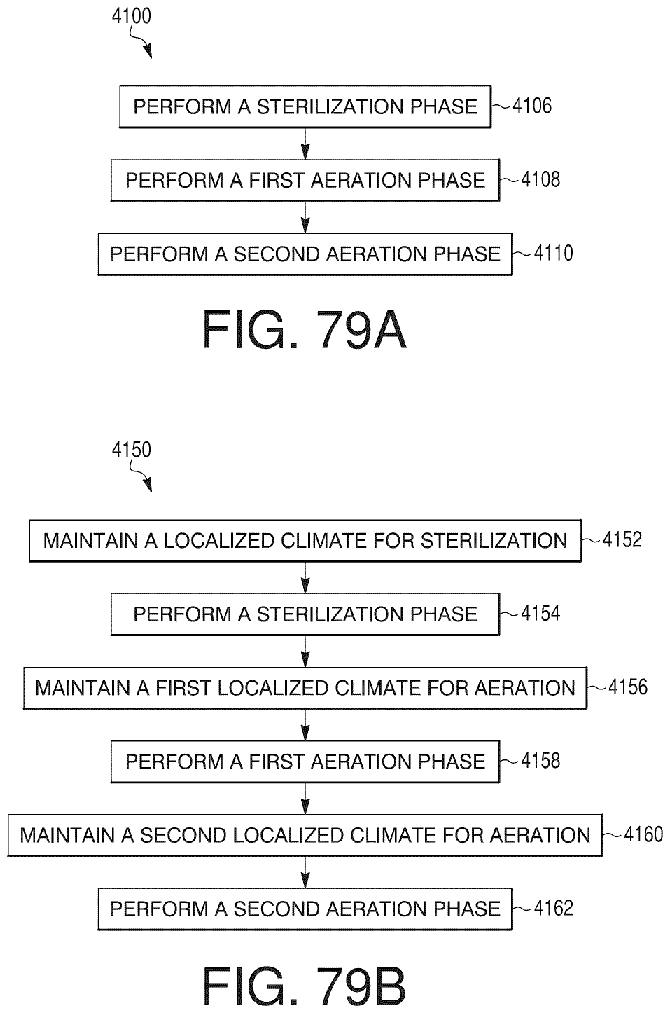

FIGS. 79A-82 depict flow diagrams of phases and steps in methods for sterilization according to the present disclosure. As will be recognized by one of ordinary skill in the art, some phases and/or steps may be omitted, combined, and/or performed out of order while remaining consistent with the present disclosure. In some embodiments, the phases and/or steps may be performed using, e.g., sterilization system 4000A, sterilization system 4000B, a variation of sterilization system 4000A, or a variation of sterilization system 4000B. Additionally or alternatively, the phases and/or steps may be applicable in other environments in which manipulation of vaporized sterilant is desirable. It will be recognized that the customizable and controllable aspects of sterilization system 4000A and/or sterilization system 4000B may be used in order to carry out phases and steps described below. For example, in some embodiments, controller 4040 may be employed to direct, adjust, or modify temperature, pressure, timings, etc. in a series of sterilization steps, setpoints, and phases performable by sterilization system 4000A and/or sterilization system 4000B. Additionally, although the phases and steps described below are recited in relation to sterilization system 4000A and/or sterilization system 4000B, one of ordinary skill in the art will understand that these phases and steps may be performed by another sterilization system, or another system having the capacity to carry out the steps.

FIG. 79A depicts a flow diagram of a series of steps in a method 4100 for sterilization in a sterilization system, such as sterilization system 4000A. According to step 4106, a sterilization phase may be performed. According to step 4108, a first aeration phase may be performed. According to step 4110, a second aeration phase may be performed. Prior to performance of the steps of method 4100, a sterilization load, such as products 4005, may be placed within a sterilization chamber, such as sterilization chamber 4002, of a sterilization system, such as sterilization system 4000A. A closed-system sterilization environment—including, for example, sterilization chamber 4002, blower exit conduit 4008, blower 4006, blower circulation conduit 4018, inlet conduit 4034, condenser 4047, and any elements connecting these components—may then be sealed. In some cases, a leak test may be performed on the closed-system sterilization environment. The leak test may include, for example, creating a vacuum through the closed system. The vacuum may be created by, e.g., expelling gas and vapor from the closed system using vacuum pump 4010. During the leak test, blower 4006 may be in operation, so as to circulate any remaining air through the closed system and create a homogenous environment. The leak test may be performed in this manner in part to verify that a suitable vacuum may be held within the closed system.

In some embodiments, the sterilization system (e.g., sterilization system 4000A) may be preconditioned. Preconditioning may include, for example, increasing the temperature of the closed system to temperatures intended to be maintained during a sterilization phase (e.g., between about 25° C. and about 50° C.). In some embodiments, preconditioning may be performed for longer than is performed in standard chemical sterilization procedures, which may allow more time for any temperature differences between the environment in the closed system (including, e.g., areas of a sterilization chamber such as sterilization chamber 4002) to decrease.

Alternately or additionally, preconditioning may include, for example, pairing a temperature of a temperature control jacket (e.g., temperature control jacket 4004) with a temperature of an inlet, such as distribution manifold 4007a, distribution manifold 4007b, and/or inlet 4009, prior to or for the duration of method 4100. By “pairing” the temperature of the temperature control jacket with the temperature of an inlet, it is meant that the temperature control jacket is programmed to maintain the same or a similar temperature as a surface temperature of an inlet inside the sterilization chamber. This may be advantageous because inlets (e.g., distribution manifolds 4007a, 4007b and/or inlet 4009) may generally be cooler than other parts of the sterilization chamber, due to the temperature of, e.g., sterilant, compressed air, or other fluid traveling through them.

Additionally, as the temperature control jacket warms the sterilization chamber 4002, the periphery of the chamber (e.g., a portion of the sterilization chamber 4002 closest to the temperature control jacket) may be warmer than the middle of the sterilization chamber (e.g., farthest from the temperature control jacket). Thus, pairing the temperature control jacket with the temperature of an inlet may reduce temperature differences across the sterilization chamber. In some embodiments, the temperature control jacket may be paired by, e.g., setting the temperature control jacket to a known temperature of an inlet during a sterilization cycle. In some embodiments, the temperature control jacket may be paired by, e.g., experimentally determining a temperature of an inlet during one or more sterilization cycles and setting the temperature control jacket to that temperature. In other embodiments, a digital thermometer may be disposed in contact with or near an inlet (e.g., a distribution manifold 4007a, 4007b, or inlet 4009), which may relay temperature information to a controller (e.g., controller 4040). For example one or more thermometers or temperature sensors may transmit temperature information to the controller 4040 when prompted, periodically, continuously, or dynamically (e.g., periodically during the sterilization cycle, based on monitored conditions of the chamber).

The controller 4040 may, in turn, set the temperature control jacket to the temperature sensed at or near the inlet (e.g., distribution manifolds 4007a, 4007b, or inlet 4009). As surface temperatures of distribution manifolds 4007a, 4007b, and inlet 4009 may in general be cooler than an average internal temperature of sterilization chamber 4002, pairing the temperature of the temperature control jacket 4004 with the temperatures of an inlet (e.g., distribution manifolds 4007a, 4007b, or inlet 4009) may rectify some differences in temperature existing throughout the sterilization chamber, which may, in turn, aid in distribution of sterilant throughout the sterilization chamber.

It is also contemplated that, in some embodiments, maintaining a temperature differential between the sterilization load and the surrounding closed system, creating “cold spots” may have advantages. For example, controlled condensation of vaporized sterilizing chemical (e.g., VHP) on “cold spots” of the load may concentrate the sterilizing chemical on the load and lead to more efficient diffusion of the chemical into the load, thus decreasing the overall amount of sterilizing chemical needed in the sterilization chamber 4002 to achieve effective sterilization. In such embodiments, it may be advantageous to reduce a time for preconditioning, or to eliminate preconditioning entirely.

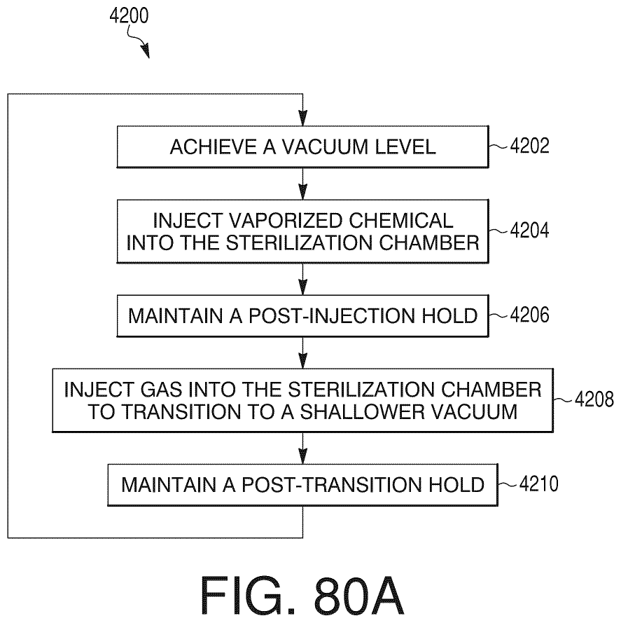

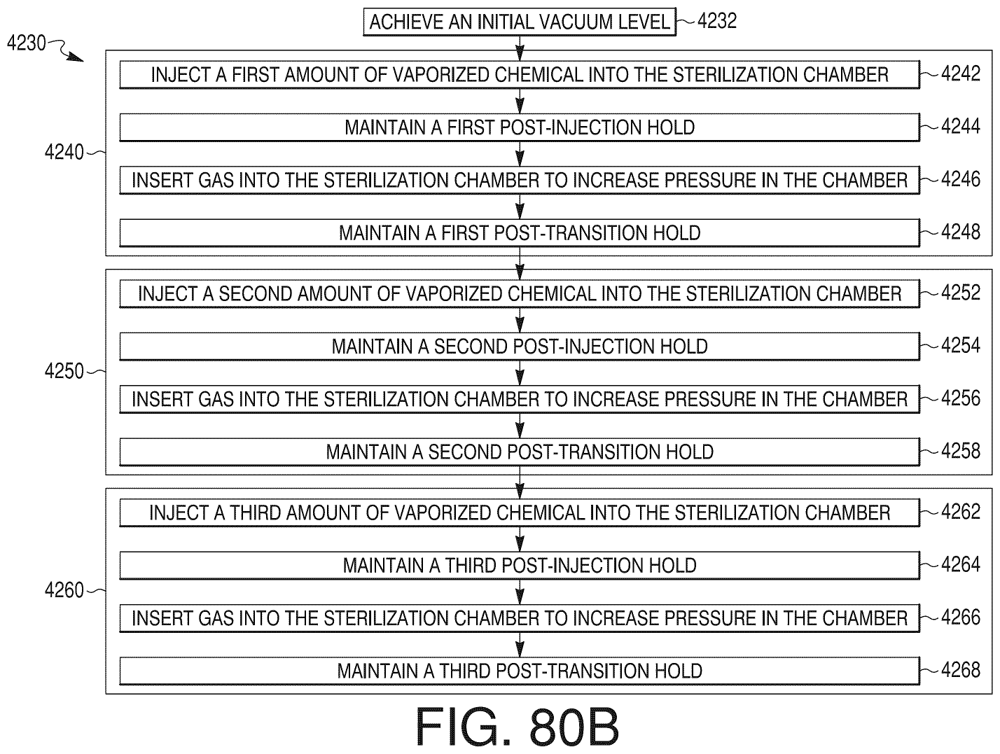

Still referring to FIG. 79A, according to step 4106, a sterilization phase may be performed. The sterilization phase may include, for example, initiating circulation of fluid through the sterilization system, achieving a vacuum level, injecting vaporized chemical into the sterilization chamber, maintaining a post-injection hold, injecting gas into the sterilization chamber to transition to a shallower vacuum, and maintaining a post-transition hold. The sterilization phase according to step 4106 may be repeated multiple times, using similar or different vacuum levels, volumes of vaporized chemicals, and/or hold times. Sterilization phases according to step 4106 are depicted in further detail in FIGS. 80A-80B.

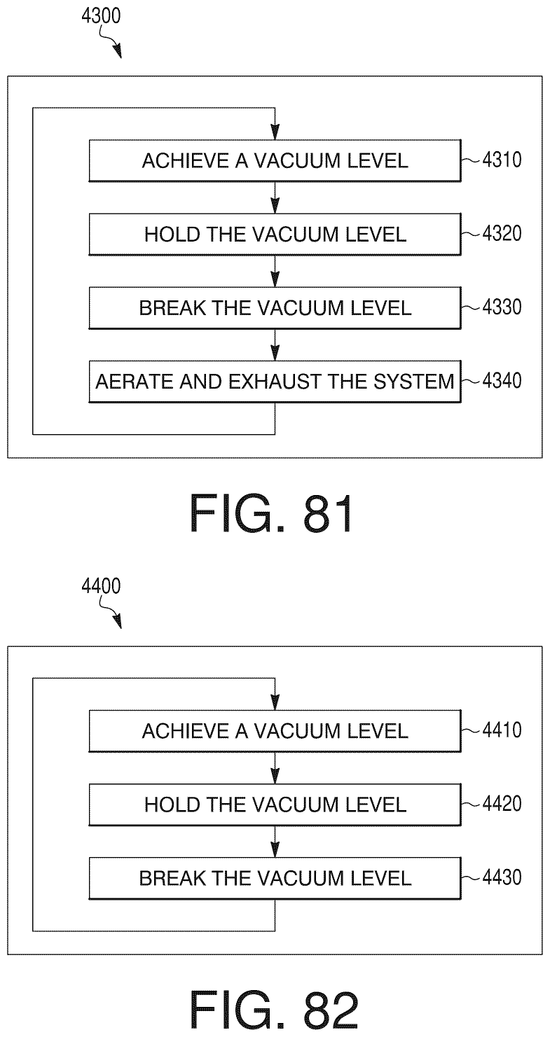

According to step 4108, a first aeration phase may be performed. The first aeration phase may include, for example, achieving a vacuum level, holding the vacuum level, breaking the vacuum level, and aerating and exhausting the system. The first aeration phase may be performed multiple times. A first aeration phase according to step 4108 is depicted in further detail in FIG. 81. According to step 4110, a second aeration phase may be performed. The second aeration phase may include, for example, achieving a vacuum level, holding the vacuum level, and breaking the vacuum level. The second aeration phase may be performed multiple times. A second aeration phase according to step 4110 is depicted in further detail in FIG. 82.

FIG. 79B depicts a flow diagram of a series of steps in a method 4150 for sterilization in a sterilization system, such as sterilization system 4000A. According to step 4152, a localized climate for sterilization may be maintained. According to step 4154, a sterilization phase may be performed. According to step 4156, a first localized climate for aeration may be maintained. According to step 4158, a first aeration phase may be performed. According to step 4160, a second localized climate for aeration may be maintained. According to step 4162, a second aeration phase may be performed.

Prior to performance of the steps of method 4150, a sterilization load may be placed within a sterilization chamber, which may be sealed, a leak test may be performed, and the sterilization system may be preconditioned, as described above with respect to method 4100. Sterilization phase 4154, first aeration phase 4158, and second aeration phase 4162 may be performed in any manner suitable for sterilization phase 4106, first aeration phase 4108, and second aeration phase 4110, respectively. Step 4158 and/or step 4162 may be performed multiple times. Additionally, while in some embodiments, step 4158 may be performed before step 4162, in alternative embodiments, step 4158 may be performed before step 4162. In some embodiments, step 4158 or step 4162 may be eliminated entirely.

Each of steps 4154, 4158 and 4162 may be preceded by the step of maintaining a localized climate. Maintaining a localized climate may generally refer to ensuring that one or more areas within a sterilization system (e.g., sterilization system 4000A) exhibit conditions (e.g., temperature, pressure, water vapor concentration, sterilant concentration, etc.) suitable for upcoming steps in a sterilization method. Maintenance of consistent or targeted localized climates may aid in robust sterilization and aeration efficacy. Localized climates may attract or repel sterilant to one or more specific localities within a system. If controlled, such localized climates may assist in achieving a desired level of sterilization. For example, inlets, and/or carts supporting the sterilization load may be heated to prevent condensation of sterilant. Sterilization system 4000A may be configured to simultaneously sterilize a plurality of carts, such as, for example, approximately 8 carts at a time. The load itself (e.g., packaging) may be kept at a temperature cooler than the carts to attract VHP or promote adhesion and condensation. Larger loads may also be used to reduce peak load temperature.

Furthermore, maintaining localized climates may aid in distributing sterilant to, or removing sterilant from, more difficult localities within a sterilization system, thus reducing the amount of “overkill” needed to sterilize or aerate those localities. Attracting sterilant to a locality that has previously proven to struggle passing sterilization standards (e.g., biological indicator metrics) may normalize sterilization of that zone without the need to add more sterilant. Similarly, removing sterilant from a locality that has previously been difficult to aerate may reduce overall aeration and drying times in the methods herein.

As depicted in FIG. 79B, a localized climate for sterilization may be maintained, e.g., step 4152. This may include attracting sterilant to an area within a system using, e.g., differences in temperature. In some embodiments, this may be accomplished by changing a temperature of one or more localities to reduce the temperature of localities to which sterilant should move. For example, the periphery of a sterilization chamber may be warmer than the middle of the chamber, due to heating elements located around the periphery of the chamber (e.g., temperature control jacket 4004 of system 4000A). Maintaining a localized climate for sterilization at the periphery of a sterilization chamber may thus include decreasing the temperature of the periphery of the sterilization chamber by, e.g., decreasing a target temperature of a heating element (e.g., temperature control jacket 4004). Maintenance of a localized climate for sterilization according to step 4152 may continue throughout the performance of the sterilization phase, according to step 4154.

A first localized climate for aeration may be maintained according to step 4156, and a second localized climate for aeration may be maintained according to step 4160. Each of the first localized climate for aeration and the second localized climate for aeration may include, e.g., raising a temperature of a locality within a sterilization system, reducing a humidity of a locality within a sterilization system, etc.. Each of the first localized climate and the second localized climate may be maintained during the first aeration phase 4158 and the second aeration phase 4162, respectively.

FIG. 80A is a flow diagram of a sterilization phase 4200, such as, for example, the sterilization phase described step 4106 of sterilization method 4100 or step 4154 of method 4150. Prior to sterilization phase 4200, a sterilization load (e.g., products 4005) may be introduced into sterilization chamber 4002. According to step 4202, a vacuum level may be achieved. According to step 4204, vaporized chemical may be injected into the sterilization chamber. According to step 4206, a post-injection hold may be maintained. According to step 4208, gas may be injected into the sterilization chamber to transition to a shallower vacuum. According to step 4210, a post-injection hold may be maintained. Sterilization phase 4200 may be repeated multiple times, such as between 2 and 15 times, between 2 and 12 times, between 2 and 10 times, between 2 and 8 times, between 2 and 6 times, between 2 and 5 times, or between 2 and 4 times, such as twice, 3 times, 4 times, 5 times, 6 times, 7 times, or 8 times. A single iteration of a sterilization phase (e.g., sterilization phase 4200) may be referred to as a “pulse.” As a part of sterilization phase 4200, a turbulent flow may be initiated and maintained in sterilization system 4000A.

According to step 4202, a vacuum level may be achieved within sterilization chamber 4002 of sterilization system 4000A. The vacuum level may be, for example, between about 400 millibars and about 700 millibars, such as between about 450 millibars and about 650 millibars, or between about 450 millibars and about 550 millibars. For example, the vacuum may be about 450 millibars, about 500 millibars, about 550 millibars, or about 600 millibars. This vacuum may promote a higher concentration of sterilizing chemical on the sterilization load, extending the amount of time at which the closed system is kept at a deeper vacuum increases exposure of the sterilization load to the sterilizing chemical. Prior to advancing to step 4204, liquid hydrogen peroxide may be injected into containers that are heated (e.g., electrically) to vaporize the liquid into VHP. In some embodiments, VHP may be produced by combining a carrier gas (e.g., a dry pharmaceutical grade compressed air) that is heated with liquid HP. In some embodiments, the amount of VHP used, or in other words the amount of VHP generated during sterilization, may be based on a weight of liquid HP that has been vaporized. In some embodiments, a feed channel (not shown) may drop decontamination liquid (e.g., liquid hydrogen peroxide) directly into corresponding holes onto the container that generates the VHP. After vaporization, step 4204 may be performed.

According to step 4204, vaporized chemical may be injected into the sterilization chamber. For example, vaporized chemical (e.g., VHP) may be injected into the sterilization chamber using air and negative pressure. In some embodiments, the vaporized chemical may include VHP. In some embodiments, the vaporized sterilization chemical may be a vaporized aqueous hydrogen peroxide solution, having a concentration of, for example, between about 5% and about 75% hydrogen peroxide by weight. In some embodiments, the vaporized chemical may be a vaporized aqueous hydrogen peroxide solution having a concentration of, for example, between about 10% and about 65% hydrogen peroxide by weight, between about 15% and about 60% hydrogen peroxide by weight, between about 30% and about 60% hydrogen peroxide by weight, or between about 45% and about 60% hydrogen peroxide by weight. In some embodiments, the vaporized chemical may be a vaporized aqueous hydrogen peroxide having a concentration of about 35% hydrogen peroxide (and 65% water) by weight. In further embodiments, the vaporized chemical may be a vaporized aqueous hydrogen peroxide having a concentration of about 59% hydrogen peroxide (and 41% water) by weight. For example, a concentration of VHP during a sterilization cycle may range from about 0.1 mg/L (0.0085 g/ft³) to about to 1.0 mg/L (0.031 g/ft³).

In some embodiments, an injected supply of VHP may be, for example, between about 50 g and about 700 g of aqueous VHP. For example, the injected supply of VHP may be between about 50 g and about 600 g, between about 100 g and about 600 g, between about 300 g and about 550 g, or between about 450 g and about 550 g. For example, the injected supply of VHP may be about 100 g, about 200 g, about 300 g, about 400 g, about 450 g, about 475 g, about 500 g, about 525 g, about 550 g, about 600 g, or about 650 g. In some embodiments, an injected supply of VHP may be quantified based on the volume or amount of load to be sterilized inside sterilization chamber 4002. For example, if a number of drug products, such as pre-filled syringes, are to be sterilized in sterilization chamber 4002, an injected supply of VHP may be between about 0.01 and about 0.15 grams of VHP per unit of the drug product inside sterilization chamber 4002, such as between about 0.01 and about 0.10 grams of VHP, such as about 0.015 grams, 0.02 grams, 0.025 grams, 0.03 grams, 0.04 grams, 0.05 grams, 0.06 grams, 0.07 grams, 0.08 grams, 0.09 grams, 0.1 grams, or 0.11 grams per drug product. In other embodiments, an injected supply of VHP may be quantified based on the volume of the sterilization environment, such as the interior of sterilization chamber 4002. For example, an injected supply of VHP may be between about 0.2 and 3.0 grams per cubic foot of volume in a sterilization chamber. For example, an injected supply of VHP may be between about 0.2 and about 2.0 grams per cubic foot, such as about 0.25 grams, about 0.50 grams, about 0.75 grams, about 1.0 gram, about 1.2 grams, about 1.4 grams, about 1.5 grams, about 1.6 grams, about 1.8 grams, or about 2.0 grams per cubic foot. In some embodiments, an injected supply of VHP may be based on a quantity of VHP injected into the sterilization chamber in a previous iteration of sterilization phase 4200. For example, a first quantity of VHP may be injected into the sterilization chamber in a first iteration of sterilization phase 4200. A second quantity of VHP, less than the first quantity, may be injected into the sterilization chamber in a second iteration of sterilization phase 4200, based on the quantity injected in the first iteration. A third quantity of VHP, less than the second quantity, may be injected into the sterilization chamber in a third iteration of sterilization phase 4200, based on the combined quantities injected in the first and second iterations. In some embodiments, the liquid hydrogen peroxide may include one or more of the following material compositions, including but not limited to, Vaprox: Fe≤0.5 ppm, Sn≤10 ppm, Phosphate ≤50 ppm, and Lead ≤4 ppm. In other embodiments, the liquid hydrogen peroxide may include one or more of the following material compositions, including but not limited to, Vaprox 59: Fe≤20 ppm, Sn≤50 ppm, Phosphate ≤200 ppm, and Lead ≤20 ppm.

In some embodiments, an injected supply of VHP may be based on a combination of the quantity of VHP in the sterilization chamber already, and a desired increase in pressure caused by the injection of additional VHP into the sterilization chamber. A desired increase in pressure caused by the injection of VHP into the sterilization chamber may be inversely related to an amount of hydrogen peroxide already present in the sterilization chamber. Advantageously, lower increases in pressure as the amount of VHP in the sterilization chamber increases may help in reducing unwanted condensation of VHP, which may be caused by excessive rises in pressure. Unwanted condensation of VHP may result in reduced sterilization efficiency and reduced aeration efficacy. In some embodiments, a pressure may be controlled after injection of VHP into the sterilization chamber relative to a threshold defining acceptable criteria. In this instance, the pressure within the sterilization chamber may be selectively raised as needed, such as after an initial hold is complete (as described herein), to maintain the pressure at the threshold for a subsequent duration.

According to step 4206, a post-injection hold may be maintained. During the post-injection hold, turbulent flow is maintained through the closed system including sterilization chamber 4002 and blower 4006. No fluids are added or removed from the closed system in which the turbulent flow is maintained. The time for which a post-injection hold is maintained (or the “post-injection hold time”) may be selected so as to allow the vaporized sterilization chemical adequate time to contact the load, without allowing the vaporized sterilization chemical to condense. In some embodiments, the post-injection hold time may be between about 2 minutes and about 20 minutes. In some embodiments, the post-injection hold time may be at least about 5 minutes, at least about 10 minutes, or at least about 15 minutes. In some embodiments, the post-injection hold time may be between about 5 minutes and about 20 minutes, between about 8 minutes and about 20 minutes, between about 10 minutes and about 20 minutes, or between about 10 minutes and about 15 minutes. In such a manner, the need for adding excess VHP into the system to ensure its contact with the sterilization load may be avoided.

According to step 4208, gas may be injected into the sterilization chamber to transition to a shallower vacuum (i.e., a higher pressure) in the sterilization chamber. The gas may be any suitable gas that can break or lessen the vacuum in sterilization chamber 4002. In some embodiments, the gas may be a dry gas, such as a gas containing nitrogen (e.g., commercially available supplies of only nitrogen or primarily nitrogen), or air having a dew point of, for example, −10° C. or colder. The use of a dry gas may be selected to allow for ample air exchange and reduction in humidity within a sterilization chamber, to further avoid unnecessary condensation. In some embodiments, gas may be injected from auxiliary ambient air supply 4030. The gas may be injected in a volume to achieve a pressure between about 500 millibars and about 1100 millibars, such as between about 550 millibars and about 1000 millibars, between about 600 millibars and about 1000 millibars, between about 700 millibars and about 900 millibars, or between about 750 millibars and about 850 millibars. For example, the second post-injection pressure may be about 700 millibars, about 750 millibars, about 800 millibars, about 850 millibars, or about 900 millibars. In cases where a sterilization load includes semi-permeable membranes through which it is desired that sterilant pass, the increase in pressure caused by step 4208 may serve to help migrate the sterilant through such semi-permeable membranes.

According to step 4210, a post-transition hold may be maintained. During the post-transition hold, the pressure achieved during step 4208 may be maintained for, for example, at least about 5 minutes, at least about 10 minutes, or at least about 15 minutes. In some embodiments, the second post-injection pressure may be maintained for between about 5 minutes and about 20 minutes, between about 8 minutes and about 20 minutes, between about 10 minutes and about 20 minutes, or between about 10 minutes and about 15 minutes.

In some embodiments, the number of times that sterilization phase 4200 may be repeated (e.g., the number of pulses) may be inversely proportional to the time that the post-injection hold is maintained in each repetition. For example, if the time that the post-injection hold is maintained is short (e.g., 10 minutes), then steps 4110 through 4116 may be repeated a greater number of times. In some embodiments, the post-injection hold is maintained for a longer period of time (e.g., 15-20 minutes), to increase the time during which the sterilization load is exposed to the sterilizing chemical in each repetition of sterilization phase 4200. In further embodiments, the number of times that sterilization phase 4200 may be repeated may depend on a total desired amount of VHP for the sterilization process. In some embodiments, for example, injection of a total amount of at least 200 g of VHP may be desired. For example, in some embodiments, injection of a total amount of at least 250 g may be desired. In some embodiments, injection of a total amount of between about 200 g and about 700 g of VHP may be desired. In some embodiments, the number of times that the sterilization phase 4200 may be repeated may depend on a combination of factors to ensure thorough permeation of VHP through a sterilization load, and to ensure enough contact time between the hydrogen peroxide and the load to permit sterilization.

FIG. 80B is a flow diagram of a sterilization phase 4230 that includes multiple sterilization pulses 4240, 4250, 4260, each of which may include the injection of different amounts of VHP into the sterilization chamber, may include different hold times and pressure changes, and may be performed once or multiple times. In some embodiments, multiple sterilization pulses 4240, 4250, 4260, may use a uniform pressure to introduce the sterilant (VHP) to sterilize the device, as described in further detail below. According to step 4232, an initial vacuum level may be achieved. During pulse 4240, a first amount of vaporized chemical may be injected into the sterilization chamber (step 4242), a first post-injection hold may be maintained (step 4244), gas may be injected into the sterilization chamber to increase pressure in the chamber (step 4246), and a first post-transition hold may be maintained (step 4248). During pulse 4250, a second amount of vaporized chemical may be injected into the sterilization chamber (step 4252), a second post-injection hold may be maintained (step 4254), gas may be injected into the sterilization chamber to increase pressure in the chamber (step 4256), and a second post-transition hold may be maintained (step 4258). During pulse 4260, a third amount of vaporized chemical may be injected into the sterilization chamber (step 4262), a third post-injection hold may be maintained (step 4264), gas may be injected into the sterilization chamber to increase pressure in the chamber (step 4266), and a third post-transition hold may be maintained (step 4268).

As described previously, the pressure increase caused by each sterilization pulse (pulses 4240, 4250, 4260) may be reflective of the sterilization chamber's existing concentration of sterilant and water (e.g., hydrogen peroxide). When the existing concentration is lower (or zero), such as prior to pulse 4240, greater increases in pressure may be used to maximize the speed at which sterilant is introduced to the load. Thus, a first amount of vaporized chemical introduced according to step 4242 may be greater than a second or third amount of vaporized chemical introduced according to steps 4252 or 4262.

In cases in which a semi-permeable membrane is desired to be traversed by sterilant (e.g., a Tyvek® membrane covering a medical device or product), a delay in transport of some sterilants (e.g., hydrogen peroxide) across the membrane has been observed. Additionally, in some sterilization cycles, hydrogen peroxide concentration in areas obstructed by a semi-permeable membrane has been observed to be generally lower than (or “dampened”) in areas not obstructed by a semi-permeable membrane. Water does not exhibit such delayed transport or dampening. As a result, sterilant strength and efficacy (and particularly, hydrogen peroxide strength and efficacy) in areas obstructed by a semi-permeable membrane may be decreased. In some embodiments, the semi-permeable membrane is sealed to the packaging during an entirety of the sterilization process, such that the packaging is not opened during the sterilization process. The third amount of vaporized chemical introduced according to step 4262 may specifically aid in overcoming this transport delay and dampened sterilant concentration. The third amount of vaporized chemical may be less than the first amount of vaporized chemical introduced according to step 4242 or the second amount of vaporized chemical introduced according to step 4244, to avoid causing aggressive increases in pressure and over-condensation which may prevent migration of sterilant across semi-permeable membranes.

Given the principles described above, in some embodiments, the first amount of sterilant injected according to step 4242 may be greater than the second amount of sterilant injected according to step 4252, and the second amount of sterilant injected according to step 4252 may be greater than the third amount of sterilant according to step 4262. For example, the first amount of sterilant may be a bolus (e.g., a large amount) to establish a lethal concentration within the sterilization chamber, the second amount (e.g., a medium amount) may be chosen to maintain the concentration and satisfy hold time requirements, and the third amount (e.g., a small amount) may be chosen to overcome sterilant transport delay and damping across semi-permeable membranes. For example, a large amount may include 15 grams of 35 wt. % H₂O₂ per cubic meter of sterilization chamber volume, a medium amount may include 7.5 grams of 35 wt. % H₂O₂ per cubic meter of sterilization chamber volume, and a small amount may be 0.5 grams of 35 wt. % H₂O₂ per cubic meter of sterilization chamber volume. In some embodiments, the total amount of H₂O₂ dose during sterilization may be greater than 500 grams.

A time for each post-injection hold 4244, 4254, 4264 may be dependent on a volume of vaporized sterilant within the sterilization chamber prior to the post-injection hold, as well as the pressure within the sterilization chamber prior to the post-injection hold. In some embodiments, each post-injection hold 4244, 4254, 4264 may be shorter than the time required for condensation of vaporized sterilant within the sterilization chamber. This may allow for more sterilant to remain in vapor form when gas is injected into the sterilization chamber according to steps 4246, 4256, 4266. For example, each post-injection hold 4244, 4254, 4264 may have a duration of ten minutes or less, such as, for example, 8 minutes or less, 6 minutes or less, 4 minutes or less, 3 minutes or less, or less than 3 minutes. The time for each post-transition hold 4248, 4258, 4268 may be sufficient to expose surfaces within the sterilization chamber to the sterilant, to effectively sterilize such surfaces. For example, each post-transition hold 4248, 4258, 4268 may have a duration of ten minutes or less, such as, for example, 8 minutes or less, 6 minutes or less, 4 minutes or less, 3 minutes or less, or less than 3 minutes. In some embodiments, a post-transition hold may cease after about half of the peak VHP concentration is reached. In some embodiments, the sterilization process described herein may cease supply and draw away VHP upon detecting an occurrence of one or more predetermined events, such as failure of a parameter from maintaining a predetermined threshold.

As previously mentioned, each of pulses 4240, 4250, and 4260 may be performed one time or multiple times. In some embodiments, pulse 4240 may be performed once, and pulses 4250 and 4260 may each be performed multiple times. For example, pulse 4240 may be repeated once or twice, pulse 4250 may be repeated once or twice, and pulse 4260 may be repeated 2-10 times. In some embodiments, either pulse 4250 or pulse 4260 may be eliminated from sterilization phase 4230. For sterilization loads including more semi-permeable membranes or materials resistant to VHP saturation, more pulses 4240 including a large bolus of sterilant may be used. During a sterilization phase, such as, for example, sterilization phase 4106, sterilization phase 4154, sterilization phase 4200, and/or sterilization phase 4230, it may be beneficial to regulate the speed with which pressure changes are accomplished such that decreases in pressure are slower than increases in pressure. For example, steps 4204, 4208, 4242, 4246, 4252, 4256, 4262, and 4266, which include or result in raising pressure within a sterilization chamber (e.g., adding fluid, breaking vacuum, or via a diaphragm or piston 4050 within sterilization chamber 4002), may include increasing pressure within a sterilization chamber more quickly than pressure is decreased in steps 4202 or 4232, which include achieving a vacuum level. This may encourage permeation of sterilant throughout the sterilization chamber and the load, including portions of the load enclosed within a semi-permeable membrane.

In some embodiments, a determination may be made as to whether any residual water exits in the container housing, where the determination may include a visual inspection for residual water vapor. During the sterilization pulses, such as pulses 4240, 4250, 4260 described in detail above, one or more of the following parameters, setpoint ranges, and/or target thresholds may be maintained and/or experienced during the sterilization process.

| Parameter | Setpoint Range | | Target |
|---|---|---|---|
| Pulse Quantity | 1-99 | pcs | 5 |
| Vacuum Level | 500-600 | mbar | 500 |
| H₂O₂ Injection | 5-150 | g | 125 |
| Injection Cycle | 0-9990 | msec | 3500 |
| SV4 Delay | 0-9990 | msec | 500 |
| SV4 On | 0-9990 | msec | 700 |
| PV231 Delay | 0-9990 | msec | 0 |
| PV231 On | 0-9990 | msec | 1500 |
| Post-Injection Hold Time | 0-100 | min | 10 |
| Post-Transition Pressure Point | 510-940 | mbar | 800 |
| Post-Transition Hold Time | 0-100 | min | 10 |

FIG. 81 is a flow diagram of a first aeration phase 4300 that may be performed as step 4108 of sterilization method 4100, after performing one or more repetitions of sterilization phase according to step 4106. According to step 4310, a vacuum level may be achieved. According to step 4320, the vacuum level may be held. According to step 4330, the vacuum level may be broken. According to step 4340, the sterilization system (e.g., sterilization system 4000A) may be aerated and exhausted.

According to step 4310, a vacuum level may be achieved in sterilization chamber 4002, while also injecting dry gas into sterilization chamber 4002 near upper interior 4001 of sterilization chamber 4002, such as via distribution manifold 4007a or inlet 4009 and/or near lower interior 4003 of sterilization chamber 4002 via distribution manifold 4007b. Dry gas may assist in allowing for air exchange without promoting condensation of sterilant. The dry gas may include, for example, oxygen and/or nitrogen. The dry gas may have a dew point of, for example, −10° C. or lower. The dry gas may be injected from, e.g., auxiliary ambient air supply 4030 or dry air supply 4027. While dry gas is being injected into sterilization chamber 4002, a vacuum may be pulled by, e.g., vacuum pump 4010 via vacuum conduit 4012, catalytic converter 4015, and vacuum exhaust conduit 4014. The vacuum may be pulled at a greater rate than the rate of injection of dry gas, such that a vacuum level is gradually achieved. The vacuum level may be, for example, between about 500 millibars and about 850 millibars, such as between about 500 millibars and about 800 millibars, between about 550 millibars and about 750 millibars, or between about 600 millibars and about 700 millibars. For example, the vacuum level may be 500 millibars, 550 millibars, 600 millibars, 650 millibars, or 700 millibars. Injection of the dry gas near upper interior 4001 of sterilization chamber 4002 while achieving a desired vacuum level reduces condensation of VHP and water vapor at upper interior 4001 of the chamber, and promotes the movement of denser molecules in sterilization chamber towards the lower interior (e.g., lower interior 4003) of sterilization chamber 4002, and to some extent out of sterilization system 4000A through vacuum exhaust conduit 4014.

According to step 4320, injection of dry gas may be stopped and the vacuum level may be held for, e.g., between about 1 minute and about 20 minutes, such as between about 2 min and about 20 min, between about 5 min and about 20 min, between about 5 min and about 15 min, or between about 5 min and about 10 min. For example, the vacuum level may be maintained for about 2, 5, 8, 10, or 15 minutes. Holding the vacuum level may continue to promote settling of denser molecules (e.g., sterilization chemical molecules) down towards the lower interior 4003 of sterilization chamber 4002, and away from the sterilization load.

According to step 4330, the vacuum level may be broken by the addition of more dry gas near upper interior 4001 of sterilization chamber 4002, via, for example, distribution manifold 4007a or inlet 4009 or dry gas near lower interior 4003 of sterilization chamber 4002 via distribution manifold 4007b. A volume of dry gas sufficient to achieve a higher pressure may be added. The higher pressure may be, for example, between 50 and 200 millibars higher than the vacuum level achieved in step 4310. The addition of more dry gas may continue to force sterilization chemicals to settle to the lower interior 4003 of sterilization chamber 4002, thus moving them away from the sterilization load and positioning them for removal via vacuum conduit 4012 or blower exit conduit 4008.

According to step 4340, the sterilization system (e.g., sterilization system 4000A) may be aerated and exhausted. During this step, blower 4006 may be turned on while recirculation valve 4019 is closed and exhaust valve 4020 is opened, such that blower 4006 pulls fluid from within sterilization chamber 4002 and expels it through exhaust 4016 via catalytic converter 4021. Because blower exit conduit 4008 is connected to sterilization chamber 4002 at lower interior 4003 of sterilization chamber 4002, denser fluids that have settled to lower interior 4003 (such as sterilizing chemicals) may be removed by this step. Air (e.g., from moist makeup air supply 4017 or dry air supply 4027) may be concurrently allowed to vent into sterilization chamber 4002, such that the pressure in sterilization chamber 4002 returns to, or near, atmospheric pressure. First aeration phase 4300 may be repeated, for example, between 1 and 35 times, such as 2, 5, 10, 15, 17, 19, 22, 25, 27, 29, 30, 32, or 35 times. Repetition of first aeration phase 4300 may ensure that the majority of sterilization chemical (e.g., VHP) is removed from sterilization system 4000A.

FIG. 82 is a flow diagram of a second aeration phase 4400 that may be performed as step 4110 of sterilization method 4100. According to step 4410, a vacuum level may be achieved. According to step 4420, a vacuum level may be held. According to step 4440, the vacuum level may be broken. According to step 4410, a vacuum level may be achieved in sterilization chamber 4002. Like with the first aeration phase, the vacuum level achieved in this phase may be, for example, between about 500 millibars and about 850 millibars, such as between about 500 millibars and about 800 millibars, between about 550 millibars and about 750 millibars, or between about 600 millibars and about 700 millibars. For example, the vacuum level may be 500 millibars, 550 millibars, 600 millibars, 650 millibars, or 700 millibars. Achieving a vacuum level may promote removing of moisture from sterilization chamber 4002 and thus the sterilization load. Thus, the sterilization load may be dried.

According to step 4420, the vacuum level may be held for, e.g., between about 1 minute and about 20 minutes, such as between about 2 min and about 20 min, between about 5 min and about 20 min, between about 5 min and about 15 min, or between about 5 min and about 10 min. For example, the vacuum level may be maintained for about 2, 5, 8, 10, or 15 minutes. Holding the vacuum level may continue to promote removal of moisture from sterilization chamber 4002, and thus the sterilization load. Thus, the sterilization load may be further dried. In some embodiments, step 4420 may be omitted.

According to step 4430, the vacuum level in sterilization chamber 4002 may be broken, or raised to a higher pressure, by the addition of dry gas from, e.g., auxiliary ambient air supply 4030 and/or dry air supply 4027. Second aeration phase 4400 may be repeated, for example, between 1 and 50 times, such as 2, 5, 10, 15, 20, 25, 30, 35, 38, 40, 42, 45, 47, 49, or 50 times. Repetition of second aeration phase 4400 may ensure drying of sterilization chamber 4002 and the sterilization load.

As has been previously described, second aeration phase 4400 may be performed either before or after first aeration phase 4300. First aeration phase 4300 may ensure, for example, that the concentration of sterilizing chemical (e.g., VHP) in sterilization chamber 4002 is relatively low, and second aeration phase 4400 may ensure that the sterilization load is dried, and may also remove residual sterilizing chemical remaining in sterilization chamber 4002 after first aeration phase 4300. In cases where second aeration phase 4400 is performed after first aeration phase 4300, first aeration phase may ensure that the concentration of sterilization chemical (e.g., VHP) in sterilization chamber 4002 is relatively low so that when sterilization chamber 4002 and the sterilization load are dried in second aeration phase 4400, there is little remaining need to remove residual sterilization chemical from the sterilization system 4000A.

In some embodiments, before performing first aeration phase 4300 or second aeration phase 4400, it may be desirable to achieve and/or maintain a localized climate suitable for aeration, as previously described with respect to steps 4158 and 4162 of sterilization method 4150. In some embodiments, before performing first aeration phase 4300 or second aeration phase 4400, pressure increases to atmospheric pressure may be avoided while the concentration of sterilant in the sterilization chamber is at or near a saturation point (e.g., after a sterilization phase is complete). Immediate increases to atmospheric pressure when a sterilization chamber is saturated or nearly saturated with sterilant may cause unnecessary condensation of sterilant and, subsequently, less efficacious aeration.

Prior to or during first aeration phase 4300 or second aeration phase 4400, repetitive inflection of pressure changes (e.g., evacuation and pressurization) may be advantageous to cause physical movement of packaging components, such as envelopes, covers, and membranes. Physical movement of parts of the load post-sterilization may aid in dislodging sterilant that has adhered to the load, thus promoting effective aeration. Additionally, prior to or during first aeration phase 4300 or second aeration phase 4400, a temperature in the sterilization chamber or sterilization system may be increased. This may increase aeration efficacy.

Further, during an aeration phase (e.g., aeration phases 4108, 4110 4158, 4162, 4300, 4400), it may be beneficial to modulate the speed with which pressure changes are accomplished such that increases in pressure are slower than decreases in pressure. For example, steps 4310, 4340, and 4410, which include achieving a vacuum level and aerating/exhausting a system, may include decreasing pressure within a sterilization chamber more quickly than pressure is increased in steps 4330 and 4430, which include breaking a vacuum level. This may encourage removal of sterilant from the sterilization chamber and the load.

It should be appreciated that hydroxyl free radicals may be formed in sterilization chamber 4002 during the one or more sterilization processes described above for purposes of sterilizing the devices disposed therein. Additionally, sterilization chamber 4002 may be configured to generate condensation onto a surface(s) of the one or more devices disposed therein during the cycles described above.

In some embodiments, the temperature of the load in sterilization chamber 4002 may not exceed approximately 35° C., the minimum pressure in sterilization chamber 4002 may be at least approximately 480 mBar, and the sterilization method 4100 may last for a time period of at least approximately 3 hours. The contents of sterilization chamber 4002 may be configured to change color during sterilization. Sterilization method 4100 may be verified by the visual indication of the color change of the sterilization chamber 4002 contents. In further embodiments, sterilization method 4100 may be verified using the results of biological indicator tests, as described above. After sterilization, the medicament components may be removed from refrigeration for a period of less than approximately 120 hours.

In some embodiments, any or all of the above-described steps and phases may be executed automatically by a sterilization system (e.g., sterilization system 4000A) as directed by, e.g., controller 4040, which may be programmed or otherwise configured in advance by e.g., a user. The methods of sterilization disclosed herein may be qualified as “limited overkill” sterilization methods, in that they may ensure sterilization of a load of, e.g., PFS while minimizing impact of the sterilization method on the product. Multiple sterilization and aeration phases have been described herein. It is to be understood that characteristics, methods, or steps of any one sterilization phase may be applied to any other sterilization method described herein. Likewise, characteristics, methods, or steps of any one aeration phase described herein may be applied to any other aeration phase.

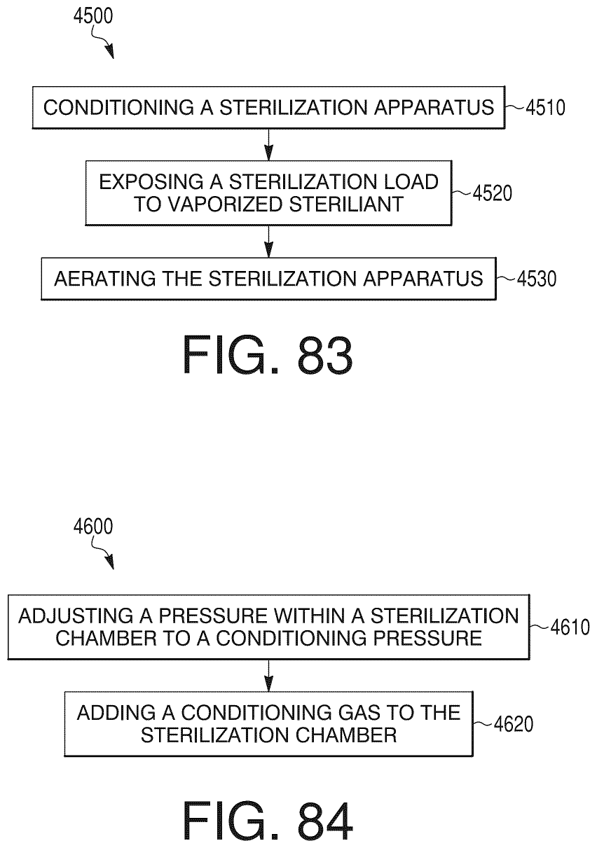

FIG. 83 depicts a flow diagram of a series of steps in an exemplary sterilization method 4500. Sterilization method 4500 may include conditioning a sterilization apparatus (method 4600). Sterilization method 4500 may include exposing a sterilization load to vaporized sterilant (method 4700). Sterilization method 4500 may include aerating the sterilization apparatus (method 4800). The sterilization method 4500 may be performed with a sterilization system, such as, for example, sterilization system 4000A and/or sterilization system 4000B.

Prior to performance of the steps of sterilization method 4500, a sterilization load, such as products 4005, may be placed within a sterilization chamber, such as sterilization chamber 4002, of a sterilization system, such as sterilization system 4000A and/or sterilization system 4000B. A closed-system sterilization environment—including, for example, sterilization chamber 4002, blower exit conduit 4008, blower 4006, blower circulation conduit 4018, inlet conduit 4034, condenser 4047, and any elements connecting these components—may then be sealed. In some cases, a leak test may be performed on the closed-system sterilization environment. The leak test may include, for example, creating a vacuum through the closed system. The vacuum may be created by, e.g., expelling gas and vapor from the closed system using vacuum pump 4010. During the leak test, blower 4006 may be in operation, so as to circulate any remaining air through the closed system and create a homogenous environment. The leak test may be performed in this manner in part to verify that a suitable vacuum may be held within the closed system.

For example, a leak test may include adjusting a pressure of sterilization chamber 4002 to a leak test pressure for a stabilization time, holding the sterilization chamber 4002 at the leak test pressure for a leak test duration, and measuring a total leak. If the pressure of the sterilization chamber 4002 decreases by more than an acceptable leak threshold, the sterilization method 4500 may be aborted and the sterilization chamber 4002 may be resealed. An acceptable total leak threshold may be about 20 millibars, about 18 millibars, about 15 millibars, about 13 millibars, about 10 millibars, about 8 millibars, or about 5 millibars. The leak test pressure may be at least 450 millibars, at least 500 millibars, at least 550 millibars, at least 600 millibars, at least 650 millibars, at least 700 millibars, at least 750 millibars, about 450 millibars, about 500 millibars, about 550 millibars, about 600 millibars, about 650 millibars, about 700 millibars, or about 750 millibars.

In some embodiments, the sterilization system (e.g., sterilization system 4000A and/or sterilization system 4000B) may be conditioned. FIG. 84 depicts a flow diagram with steps in an exemplary method 4600 of conditioning a sterilization apparatus. Conditioning a sterilization apparatus may include adjusting a pressure within a sterilization chamber (e.g., sterilization chamber 4002) to a conditioning pressure (step 4610). Suitable conditioning pressures may be at least 450 millibars, at least 500 millibars, at least 550 millibars, at least 600 millibars, at least 650 millibars, at least 700 millibars, at least 750 millibars, about 450 millibars, about 500 millibars, about 550 millibars, about 600 millibars, about 650 millibars, about 700 millibars, or about 750 millibars. Conditioning a sterilization apparatus may further include adding a conditioning gas to the sterilization chamber (step 4610). The conditioning gas may be a dry gas, such as a gas containing nitrogen (e.g., commercially available supplies of only nitrogen or primarily nitrogen), or air having a dew point of, for example, −10° C. or less. The use of a dry gas may be selected to allow for ample air exchange and reduction in humidity within a sterilization chamber (e.g., sterilization chamber 4002), to further avoid unnecessary condensation. After the conditioning gas is added, the pressure of the sterilization chamber may be at least 550 millibars, at least 600 millibars, at least 650 millibars, at least 700 millibars, at least 750 millibars, at least 800 millibars, at least 850 millibars, at least 900 millibars, at least 950 millibars, about 450 millibars, about 500 millibars, about 550 millibars, about 600 millibars, about 650 millibars, about 700 millibars, about 750 millibars, about 800 millibars, or about 850 millibars. Together, steps 4610 and 4620 of conditioning a sterilization apparatus may be referred to as a conditioning pulse. Conditioning a sterilization apparatus may include multiple conditioning pulses, such as for example, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 conditioning pulses.

In addition or alternatively, the method 4600 of conditioning may include, for example, increasing the temperature of the closed system to temperatures intended to be maintained during a sterilization phase (e.g., between about 25° C. and about 50° C.). In some embodiments, conditioning may be performed for longer than is performed in standard chemical sterilization procedures, which may allow more time for any temperature differences between the environment in the closed system (including, e.g., areas of a sterilization chamber such as sterilization chamber 4002) to decrease. Alternatively or additionally, conditioning may include, for example, pairing a temperature of a temperature control jacket (e.g., temperature control jacket 4004) with a temperature of an inlet, such as first distribution manifold 4007a, second distribution manifold 4007b, and/or inlet 4009, prior to or for the duration of method 4600. By “pairing” the temperature of the temperature control jacket with the temperature of an inlet, it is meant that the temperature control jacket is programmed to maintain the same or a similar temperature as a surface temperature of an inlet inside the sterilization chamber. This may be advantageous because inlets (e.g., distribution manifolds 4007a, 4007b and/or inlet 4009) may generally be cooler than other parts of the sterilization chamber, due to the temperature of, e.g., sterilant, compressed air, or other fluid traveling through them. Additionally, as the temperature control jacket 4004 warms the sterilization chamber 4002, the periphery of the chamber (e.g., a portion of the sterilization chamber 4002 closest to the temperature control jacket) may be warmer than the middle of the sterilization chamber (e.g., farthest from the temperature control jacket). Thus, pairing the temperature control jacket with the temperature of an inlet may reduce temperature differences across the sterilization chamber. In some embodiments, the temperature control jacket may be paired by, e.g., setting the temperature control jacket to a known temperature of an inlet during a sterilization cycle. In some embodiments, the temperature control jacket may be paired by, e.g., experimentally determining a temperature of an inlet during one or more sterilization cycles and setting the temperature control jacket to that temperature. In other embodiments, a digital thermometer may be disposed in contact with or near an inlet (e.g., a distribution manifold 4007a, 4007b, or inlet 4009), which may relay temperature information to a controller (e.g., controller 4040). For example, one or more thermometers or temperature sensors (not shown) may transmit temperature information to the controller 4040 when prompted, periodically, continuously, or dynamically (e.g., periodically during the sterilization cycle, based on monitored conditions of the chamber).

The controller 4040 may, in turn, set the temperature control jacket 4004 to the temperature sensed at or near the inlet (e.g., distribution manifolds 4007a, 4007b, or inlet 4009). As surface temperatures of distribution manifolds 4007a, 4007b, and inlet 4009 may in general be cooler than an average internal temperature of sterilization chamber 4002, pairing the temperature of the temperature control jacket 4004 with the temperatures of an inlet (e.g., distribution manifolds 4007a, 4007b, or inlet 4009) may rectify some differences in temperature existing throughout the sterilization chamber, which may, in turn, aid in distribution of sterilant throughout the sterilization chamber.

It is also contemplated that, in some embodiments, maintaining a temperature differential between the sterilization load and the surrounding closed system, creating “cold spots” via may have advantages. For example, controlled condensation of vaporized sterilizing chemical (e.g., VHP) on “cold spots” of the load may concentrate the sterilizing chemical on the load and lead to more efficient diffusion of the chemical into the load, thus decreasing the overall amount of sterilizing chemical needed in the sterilization chamber 102 to achieve effective sterilization. In such embodiments, it may be advantageous to reduce a time for preconditioning, or to eliminate preconditioning entirely.

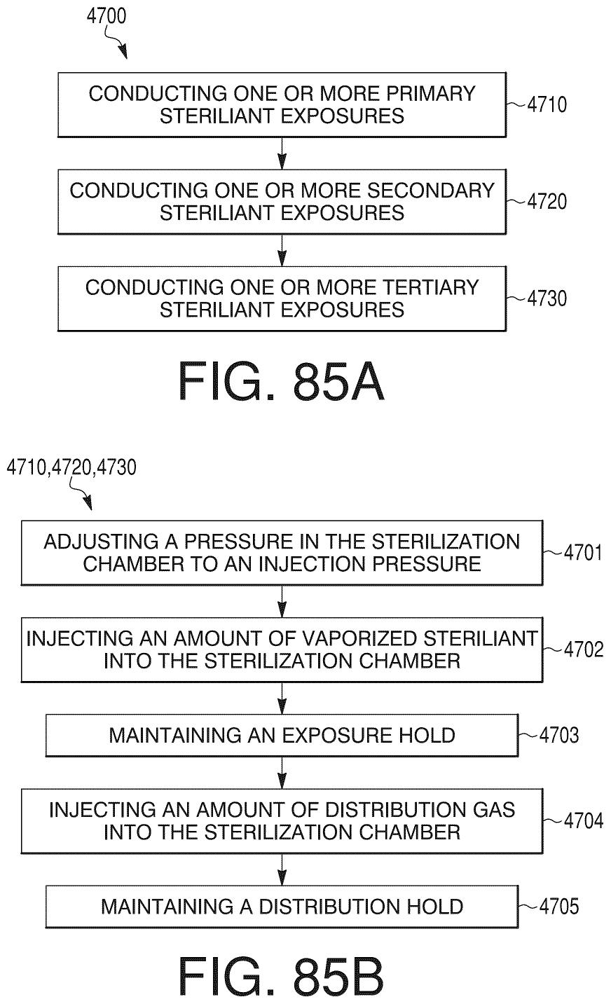

As described herein, the sterilization method 4500 may include exposing a sterilization load to vaporized sterilant. FIG. 85A depicts a flow diagram with steps in an exemplary method 4700 of exposing a sterilization load to vaporized sterilant. Exposing a sterilization load to vaporized sterilant may include conducting one or more primary sterilant exposures (step 4710), conducting one or more second sterilant exposures (step 4720), and/or conducting one or more tertiary sterilant exposures (step 4730).

FIG. 85B depicts a flow diagram with steps in an exemplary method (4710, 4720, 4730) of a sterilant exposure, such as, for example, a primary sterilant exposure, a second sterilant exposure, or a tertiary sterilant exposure. The method 4710, 4720, 4730 of the sterilant exposure may include adjusting a pressure in the sterilization chamber to an injection pressure (step 4701), injecting an amount of vaporized sterilant into the sterilization chamber (step 4702), maintaining an exposure hold (step 4703), injecting an amount of distribution gas into the sterilization chamber (step 4704), and maintaining a distribution hold (step 4705).

The injection pressure may be, for example, about 400 millibars to about 1000 millibars, about 450 millibars to about 1000 millibars, about 400 millibars to about 900 millibars, about 450 millibars to about 850 millibars, about 400 millibars to about 800 millibars, about 450 millibars to about 800 millibars, about 450 millibars to about 750 millibars. For example, the injection pressure may be about 400 millibars, about 450 millibars, about 500 millibars, about 550 millibars, or about 600 millibars. The injection pressure may promote a higher concentration of sterilant on the sterilization load, and increasing exposure of the sterilization load to the sterilant.

Injecting an amount of vaporized sterilant into the sterilization chamber may include injecting the vapor produced by vaporizing a solution including the sterilant. In some aspects, the solution including the sterilant may be an aqueous hydrogen peroxide solution, having a concentration of, for example, between about 5% and about 75% hydrogen peroxide by weight. In some embodiments, the vaporized chemical may be a vaporized aqueous hydrogen peroxide solution having a concentration of, for example, between about 10% and about 65% hydrogen peroxide by weight, between about 15% and about 60% hydrogen peroxide by weight, between about 30% and about 60% hydrogen peroxide by weight, between about 30% and about 60% hydrogen peroxide by weight, or between about 45% and about 60% hydrogen peroxide by weight. In some embodiments, the vaporized chemical may be a vaporized aqueous hydrogen peroxide having a concentration of about 35% hydrogen peroxide (and 65% water) by weight. In further embodiments, the vaporized chemical may be a vaporized aqueous hydrogen peroxide having a concentration of about 59% hydrogen peroxide (and 41% water) by weight.

The amount of vaporized chemical injected into the sterilization chamber may be characterized by the mass of solution including the sterilant that was used to generate the vapor. The injected amount of vaporized sterilant may be about 50 g to about 350 g, about 50 g to about 300 g, about 50 g to about 250 g, about 50 g to about 200 g, about 100 g to about 300 g, about 100 g to about 250 g, about 100 g to about 200 g, about 100 g to about 175 g, or about 125 g to about 200 g. For example, the injected amount of vaporized sterilant may be about 50 g, about 75 g, about 90 g, about 100 g, about 110 g, about 125 g, about 130 g, about 140 g, about 150 g, about 160 g, about 175 g, about 180 g, about 190 g, or about 200 g.

In some aspects, amount of injected sterilant may be quantified based on the volume or amount of load to be sterilized inside sterilization chamber 4002. For example, if a number of drug products, such as pre-filled syringes, are to be sterilized in sterilization chamber 4002, an injected amount of vaporized sterilant may be about 0.01 to about 0.25, about 0.01 to about 0.20, about 0.01 to about 0.15, about 0.05 to about 0.25, about 0.05 to about 0.20, about 0.05 to about 0.25, about 0.05 to about 0.15, or about 0.015 to about 0.25 grams per unit drug product. In other aspects, an amount of injected sterilant may be quantified based on the volume of the sterilization environment, such as the interior of sterilization chamber 4002. For example, an amount of injected sterilant may be about 0.25 grams, about 0.50 grams, about 0.75 grams, about 1.0 gram, about 1.2 grams, about 1.4 grams, about 1.5 grams, about 1.6 grams, about 1.8 grams, about 2.0 grams, about 2.2 grams, about 2.5 grams, or about 3 grams per cubic foot of volume in a sterilization chamber.

The total amount of vaporized sterilant injected into the sterilization chambers across all exposures of vaporized sterilant to the sterilization load may be about 600 grams to about 1000 grams, about 600 grams to about 950 grams, about 650 grams to about 1000 grams, about 650 grams to about 950 grams, about 650 grams to about 900 grams, about 700 grams to about 1000 grams, about 700 grams to about 950 grams, about 700 grams to about 900 grams, about 750 grams to about 1000 grams, about 750 grams to about 950 grams, about 750 grams to about 900 grams, about 800 grams to about 900 grams, about 600 grams, about 650 grams, about 700 grams, about 725 grams, about 750 grams, about 775 grams, about 800 grams, about 825 grams, about 850 grams, about 875 grams, about 900 grams, or about 925 grams.

Referring still to FIG. 85B, after the amount of vaporized sterilant is injected into the sterilization chamber at step 4702, an exposure hold may be maintained at step 4703. The time for which an exposure hold is maintained (an exposure hold time) may be selected so as to allow the vaporized sterilant adequate time to contact the load, without allowing excess condensation of the vaporized sterilant. In some embodiments, the exposure hold time may be at least at least 30 seconds, at least 45 seconds, at least 60 seconds, at least 75 seconds, at least 120 seconds, at least 150 seconds, at least 190 seconds, about 45 seconds, about 60 seconds, about 75 seconds, or about 90 seconds.

After the exposure hold at step 4703, a distribution gas may be injected into the sterilization chamber at step 4704. Addition of distribution gas may increase the pressure in the sterilization chamber to a transition pressure. The gas may be any suitable gas that can lessen the vacuum in sterilization chamber 4002. In some aspects, the distribution gas may be a dry gas, such as a gas containing nitrogen (e.g., commercially available supplies of only nitrogen or primarily nitrogen), or air having a dew point of, for example, −10° C. or less. The use of a dry gas may be selected to allow for ample air exchange and reduction in humidity within a sterilization chamber, to further avoid excess condensation. In some embodiments, distribution gas may be injected from ambient air supply 4017, dry air supply 4027, and/or auxiliary ambient air supply 4030. The amount of distribution gas injected may be an amount sufficient to achieve a transition pressure of about 750 millibars to about 1100 millibars, about 750 millibars to about 1100 millibars, about 750 millibars to about 1000 millibars, about 800 millibars to about 1100 millibars, about 800 millibars to about 1000 millibars, about 850 millibars to about 1000 millibars, about 850 millibars to about 950 millibars, about 900 millibars to about 1000 millibars, about 800 millibars, about 850 millibars, about 900 millibars, about 910 millibars, about 920 millibars, about 930 millibars, about 940 millibars, about 950 millibars, about 960 millibars, about 970 millibars, about 980 millibars, or about 1000 millibars, within the sterilization chamber.

In instances where a sterilization load includes semi-permeable membranes through which it is desired that sterilant pass, the increase in pressure caused by addition of the distribution gas may serve to help migrate the sterilant through such semi-permeable membranes.

After an amount of distribution gas is injected into the sterilization chamber at step 4704, a distribution hold may be maintained at step 4705. The amount of time that the distribution hold is maintained for may be referred to as the distribution hold time. During the distribution hold, the pressure achieved during from the injection distribution gas may be maintained for at least 30 seconds, at least 1 minute, at least 2 minutes, at least 3 minutes, at least 4 minutes, at least 5 minutes, at least 6 minutes, at least 7 minutes, at least 8 minutes, about 30 seconds, about 1 minute, about 2 minutes, about 3 minutes, about 4 minutes, about 5 minutes, about 6 minutes, about 7 minutes, about 8 minutes, about 9 minutes, or about 10 minutes.

A single iteration of a sterilant exposure (e.g., method 4710, 4720, 4730) may be referred to as a sterilization pulse. The one or more primary sterilant exposures may include about 2 to about 15 sterilization pulses, about 2 to about 12 sterilization pulses, about 2 to 10 sterilization pulses, about 2 to about 8 sterilization pulses, about 2 to about 6 sterilization pulses, about 2 to about 5 sterilization pulses, about 2 to about 5 sterilization pulses, such as, for example, 2 pulses, 3 pulses, 4 pulses, 5 pulses, 6 pulses, 7 pulses, or 8 pulses. The one or more secondary sterilant exposures may include about 2 to about 15 sterilization pulses, about 2 to about 12 sterilization pulses, about 2 to 10 sterilization pulses, about 2 to about 8 sterilization pulses, about 2 to about 6 sterilization pulses, about 2 to about 5 sterilization pulses, about 2 to about 5 sterilization pulses, such as, for example, 2 pulses, 3 pulses, 4 pulses, 5 pulses, 6 pulses, 7 pulses, or 8 pulses. The one or more tertiary sterilant exposures may include about 2 to about 15 sterilization pulses, about 2 to about 12 sterilization pulses, about 2 to 10 sterilization pulses, about 2 to about 8 sterilization pulses, about 2 to about 6 sterilization pulses, about 2 to about 5 sterilization pulses, about 2 to about 5 sterilization pulses, such as, for example, 2 pulses, 3 pulses, 4 pulses, 5 pulses, 6 pulses, 7 pulses, or 8 pulses.

Parameters of a sterilant exposure may include an amount of sterilant added, a duration of the sterilant injection, an exposure hold time, a transition pressure, and a distribution hold time. Each primary sterilant exposure may have the same parameters as each other primary sterilant exposure. Each secondary sterilant exposure may have the same parameters as each other secondary sterilant exposure. Each tertiary sterilant exposure have the same parameters as each other tertiary sterilant exposure. A primary sterilant exposure may have at least one parameter that is different than a secondary sterilant exposure and/or at least one parameter that is different than a tertiary sterilant exposure. In addition or alternatively, a secondary sterilant exposure may have at least one parameter that is different than a tertiary sterilant exposure.

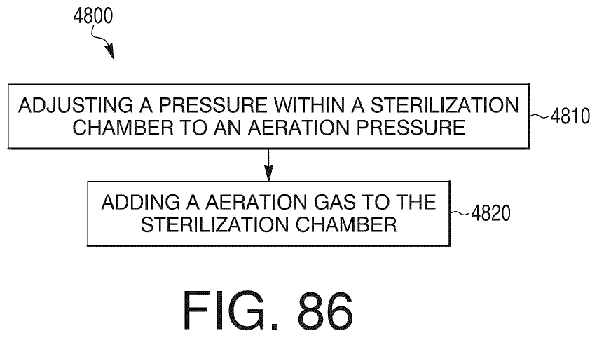

FIG. 86 depicts a flow diagram with steps in an exemplary method 4800 of aerating the sterilization apparatus. Aerating the sterilization apparatus may include adjusting a pressure within a sterilization chamber to an aeration pressure (step 4810). Aerating the sterilization apparatus may also include adding an aeration gas to the sterilization chamber (step 4820).

One iteration of adjusting a pressure within the sterilization chamber to an aeration pressure (step 4810) and adding an aeration gas to the sterilization chamber (step 4820) may be referred to as an aeration pulse. Aerating the sterilization apparatus may include multiple aeration pulses. For example, aerating the sterilization apparatus may include at least 1 aeration pulse, at least 2 aeration pulses, at least 3 aeration pulses, at least 4 aeration pulses, at least 5 aeration pulses, at least 6 aeration pulses, at least 7 aeration pulses, at least 8 aeration pulses, at least 9 aeration pulses, at least 10 aeration pulses, 1 aeration pulse, 2 aeration pulses, 3 aeration pulses, 4 aeration pulses, 5 aeration pulses, 6 aeration pulses, 7 aeration pulses, 8 aeration pulses, 9 aeration pulses, or 10 aeration pulses.

The aeration pressure may be about 400 millibars to about 1000 millibars, about 400 millibars to about 900 millibars, about 400 millibars to about 600 millibars, about 450 millibars to about 900 millibars, about 450 millibars to about 600 millibars, about 450 millibars to about 550 millibars, at least 400 millibars, at least 450 millibars, at least 475 millibars, at least 500 millibars, at least 525 millibars, at least 550 millibars, at least 575 millibars, at least 600 millibars, about 400 millibars, about 450 millibars, about 475 millibars, about 500 millibars, about 525 millibars, about 550 millibars, about 575 millibars, or about 600 millibars.

The aeration gas may comprise a dry gas, such as a gas containing nitrogen (e.g., commercially available supplies of only nitrogen or primarily nitrogen), or air having a dew point of, for example, −10° C. or less. Adding an aeration gas to the sterilization chamber may raise the pressure of the sterilization chamber. In some aspects, adding the aeration gas to the sterilization chamber may raise the pressure of the sterilization chamber by about 50 millibar to about 150 millibar to an exhaust pressure. For example, after the aeration gas is added to the sterilization chamber at step 4820, the exhaust pressure of the sterilization chamber may be 500 millibars to about 1100 millibars, about 500 millibars to about 1000 millibars, about 500 millibars to about 700 millibars, about 550 millibars to about 1000 millibars, about 550 millibars to about 700 millibars, about 550 millibars to about 650 millibars, at least 500 millibars, at least 550 millibars, at least 575 millibars, at least 600 millibars, at least 625 millibars, at least 650 millibars, at least 675 millibars, at least 800 millibars, about 500 millibars, about 550 millibars, about 575 millibars, about 600 millibars, about 625 millibars, about 650 millibars, about 675 millibars, or about 700 millibars.

After the pressure in the sterilization chamber is raised by the addition of the aeration gas a step 4820, the pressure in the sterilization chamber may need to be adjusted to an aeration pressure (step 4810) for a subsequent aeration pulse. The pressure may be lowered by venting the sterilization apparatus, and exhausting gases within the sterilization chamber. During this step, blower 4006 may be turned on while recirculation valve 4019 is closed and exhaust valve 4020 is opened, such that blower 4006 pulls fluid from within sterilization chamber 4002 and expels it through exhaust 4016 via catalytic converter 4021. Because blower exit conduit 4008 is connected to sterilization chamber 4002 at lower interior 4003 of sterilization chamber 102, denser fluids that have settled to lower interior 4003 (such as sterilizing chemicals) may be removed by this step. Air (e.g., from ambient air supply 4017, dry air supply 4027, and/or auxiliary ambient air supply 4030) may be concurrently allowed to vent into sterilization chamber 4002.

The amount of time required to transition the pressure in the sterilization chamber from the pressure resulting from the addition of the aeration gas to the aeration pressure (step 4820) of the subsequent aeration pulse may be referred to as an exhaust time. An exhaust time may be instantaneous (i.e., an exhaust time of 0 minutes), or the decrease in pressure may be more gradual. For example, the exhaust time of an aeration pulse may be at least 1 minute, at least 3 minutes, at least 5 minutes, at least 7 minutes, at least 10 minutes, at least 12 minutes, at least 15 minutes, at least 18 minutes, at least 20 minutes, at least 22 minutes, at least 25 minutes, at least 28 minutes, at least 30 minutes, at least 32 minutes, at least 35 minutes, at least 37 minutes, at least 40 minutes, at least 45 minutes, at least 50 minutes, at least 55 minutes, at least 60 minutes, about 1 minute, about 3 minutes, about 5 minutes, about 7 minutes, about 10 minutes, about 12 minutes, about 15 minutes, about 18 minutes, about 20 minutes, about 22 minutes, about 25 minutes, about 28 minutes, about 30 minutes, about 32 minutes, about 35 minutes, about 37 minutes, about 40 minutes, about 45 minutes, about 50 minutes, about 55 minutes, or about 60 minutes.

Parameters of an aeration pulse may include a sterilization pressure, an amount of aeration gas added, a pressure achieved by the addition of the aeration gas, and/or an exhaust time. Each aeration pulse may have the same parameters as each other aeration pulse. An aeration pulse may have different parameters than a previous or subsequent aeration pulse. An aeration pulse may have the same parameters as a previous and/or subsequent aeration pulse.

In some methods, the pressure of the sterilization chamber 4002 may be adjusted (step 4810) while the aeration gas is added to the sterilization chamber 4002 (step 4820). For example, a vacuum may be drawn in the sterilization chamber 4002 while also injecting aeration gas into sterilization chamber 4002 near upper interior 4001 of sterilization chamber 4002, such as via first distribution manifold 4007a or inlet 4009 and/or near lower interior 4003 of sterilization chamber 4002 via second distribution manifold 4007b. The aeration gas may be injected from, for example, ambient air supply 4017, dry air supply 4027, and/or auxiliary ambient air supply 4030. While dry gas is being injected into sterilization chamber 4002, a vacuum may be pulled by, e.g., vacuum pump 4010 via vacuum conduit 4012, catalytic converter 4015, and vacuum exhaust conduit 4014. The vacuum may be pulled at a greater rate than the rate of injection of dry gas, such that a vacuum level is gradually achieved. Injection of the dry gas near upper interior 4001 of sterilization chamber 4002 while achieving a desired vacuum level reduces condensation of VHP and water vapor at upper interior 4001 of the sterilization chamber 4002, and promotes the movement of denser molecules in sterilization chamber 4002 towards the lower interior (e.g., lower interior 4003) of sterilization chamber 4002, and to some extent out of sterilization system 4000A through vacuum exhaust conduit 4014. The addition of more dry gas may continue to force sterilization chemicals to settle to the lower interior 4003 of sterilization chamber 4002, thus moving them away from the sterilization load and positioning them for removal via vacuum conduit 4012 or blower exit conduit 4008.

In some methods, before performing a sterilization or aeration pulses, it may be desirable to achieve and/or maintain a localized climate suitable for sterilization or aeration. Maintaining a localized climate may generally refer to ensuring that one or more areas within a sterilization system (e.g., sterilization system 4000A and/or sterilization system 4000B) exhibit conditions (e.g., temperature, pressure, water vapor concentration, sterilant concentration, etc.) suitable for upcoming steps in a sterilization method. Maintenance of consistent or targeted localized climates may aid in robust sterilization and aeration efficacy. Localized climates may attract or repel sterilant to one or more specific localities within a system. If controlled, such localized climates may assist in achieving a desired level of sterilization. For example, inlets and/or carts supporting the sterilization load may be heated to prevent condensation of sterilant. The sterilization load may be kept at a temperature cooler than the carts to attract VHP or promote adhesion and condensation. Larger loads may also be used to reduce peak load temperature.

Furthermore, maintaining localized climates may aid in distributing sterilant to, or removing sterilant from, more difficult localities within a sterilization system, thus reducing the amount of “overkill” needed to sterilize or aerate those localities. These localities may include surfaces and interior regions of packaging materials. Attracting sterilant to a locality that has previously proven to struggle passing sterilization standards (e.g., biological indicator metrics) may normalize sterilization of that zone without the need to add more sterilant. Similarly, removing sterilant from a locality that has previously been difficult to aerate may reduce overall aeration and drying times in the methods described herein.

Maintaining localized climates may be accomplished by changing a temperature of one or more localities to reduce the temperature of localities to which sterilant should move. For example, the periphery of a sterilization chamber may be warmer than the middle of the chamber, due to heating elements located around the periphery of the chamber (e.g., temperature control jacket 4004). Maintaining a localized climate for sterilization at the periphery of a sterilization chamber may thus include decreasing the temperature of the periphery of the sterilization chamber by, e.g., decreasing a target temperature of a heating element (e.g., temperature control jacket 4004). In some examples, prior to or during an aeration pulse, a temperature in the sterilization chamber or sterilization system may be increased. This may increase movement of sterilant out of the sterilization chamber 4002.

In some embodiments, any or all of the above-described steps and phases may be executed automatically by a sterilization system (e.g., sterilization system 4000A and/or sterilization system 4000B) as directed by, e.g., controller 4040, which may be programmed or otherwise configured in advance by e.g., a user. The methods of sterilization disclosed herein may be qualified as “limited overkill” sterilization methods, in that they may ensure sterilization of a load of, e.g., PFS while minimizing impact of the sterilization method on the product.

During operation of a sterilization method (e.g., method 4500), conditions experienced by the sterilization load may be monitored to confirm that an effective sterilization occurred without damaging the drug substance within the sterilization load. Conditions that may be monitored include total amount of sterilant injected into the sterilization chamber 4002, a maximum temperature of the sterilization load, a minimum pressure of the sterilization load, and/or a total duration of the exposure to vaporized sterilant.

Chemical indicators may be placed throughout the sterilization load to ensure exposure of vaporized sterilant to all portions of the sterilization load. In addition or alternatively, biological indicators may be placed throughout the sterilization load to ensure effectiveness of the vaporized sterilant to all portions of the sterilization load. Chemical indicators may be configured to change color when exposed to a threshold amount of a specific sterilant. Biological indicators may include one or more microorganisms, including, for example, microorganisms with a tolerance for a specific sterilant. In the context of sterilization methods utilizing vaporized hydrogen peroxide, biological indicators may include Geobacillus stearothermophilus cultures with spores that have hydrogen peroxide resistance. After their use in a sterilization method, the biological indicators may be tested in a lab for growth to confirm effectiveness of the sterilization method.

In some examples, a sterilization method may be considered to have effectively sterilized the sterilization load without adversely affecting drug product in the load when one or more of the following conditions are met: the maximum temperature of the sterilization load did not exceed 35° C., the minimum pressure of the sterilization chamber did not go below 480 millibars, the total duration of exposure of the sterilization load to the vaporized sterilant was at least 3 hours, all chemical indicators change color, and/or all biological indicators show negative growth.

Multiple sterilization and aeration phases have been described herein. It is to be understood that characteristics, methods, or steps of any one sterilization phase may be applied to any other sterilization method described herein. Likewise, characteristics, methods, or steps of any one aeration phase described herein may be applied to any other aeration phase or method.

### EXAMPLES

The following examples are intended to illustrate the present disclosure without being limiting in nature. It is understood that the present disclosure encompasses additional aspects and embodiments consistent with the foregoing description and following examples.

### Example 1

In one example, a sterilization load including 24 biological indicators was loaded in a sterilization chamber, and a sterilization method was performed. The sterilization method included a leak test, a preconditioning phase, sterilization phase with one sterilization pulse, and two aeration phases. The exact parameters for the sterilization method are summarized in Table 1.

TABLE 1

| Phase | Parameter | Value | |
|---|---|---|---|
| Leak Test | Vacuum Level | 500 | mbar |
| Leak Test | Stabilization Time | 2 | minutes |
| Leak Test | Leak Test Time | 5 | minutes |
| Leak Test | Acceptable Total Leak | 13 | mbar |
| Pre-conditioning | Pulse Quantity | 12 | |
| Pre-conditioning | Vacuum Level | 500 | mbar |
| Pre-conditioning | Vacuum Break Point | 700 | mbar |
| Pre-conditioning | Jacket and Doors | 28° | C. |
| | Temperature | | |
| Pre-conditioning | Vaporizer Temperature | 110° | C. |
| Sterilization | Pulse Quantity | 1 | |
| Sterilization | Vacuum Level | 500 | mbar |
| Sterilization | H₂O₂ (59 wt. % solution) | 150 | g/pulse |
| | Injection Amount | | |
| Sterilization | Injection Cycle Duration | 4500 | milliseconds |
| Sterilization | Post-Injection Hold Time | 2 | minutes |
| Sterilization | Transition Pressure Point | 940 | mbar |
| Sterilization | Post-transition Hold Time | 7 | minutes |
| First Aeration | Pulse Quantity | 10 | |
| First Aeration | Vacuum Level | 500 | mbar |
| First Aeration | Vacuum Break Point | 899 | mbar |
| First Aeration | Exhaust Time | 0 | minutes |

| | | |
|---|---|---|
| First Aeration | Recirculate During Vacuum | Yes |
| Second Aeration | Pulse Quantity | 10 |
| Second Aeration | Vacuum Level | 500 |
| Second Aeration | Vacuum Break Point | 900 |
| Second Aeration | Exhaust Time | 10 |
| Second Aeration | Recirculate During Vacuum | No |

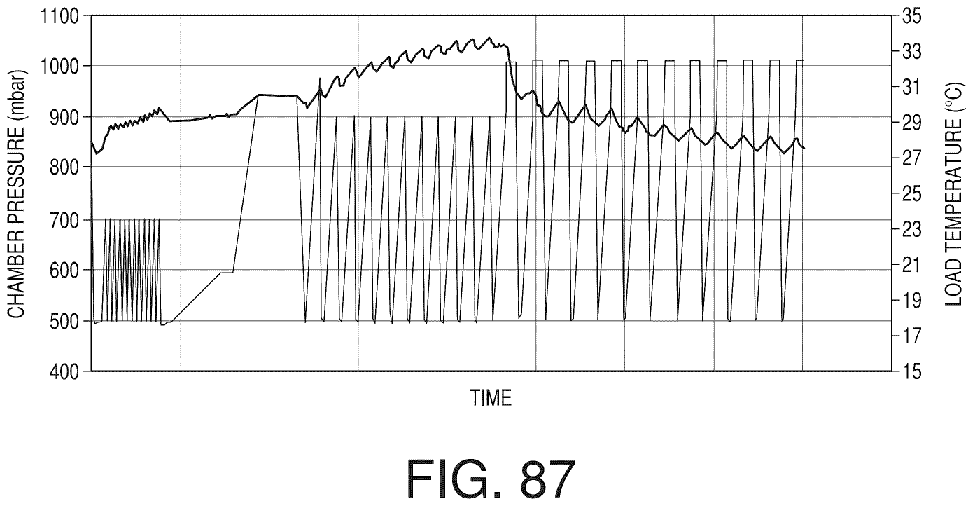

The pressure of the sterilization chamber and the temperature of the load, during the sterilization method of Example 1, were measured and are shown in FIG. 87.

### Example 2

In another example, a sterilization load including 20 biological indicators was loaded in a sterilization chamber, and a sterilization method was performed. The sterilization method included a leak test, a preconditioning phase, and a sterilization phase with two sterilization pulses. The exact parameters for the sterilization method are summarized in Table 2. The temperature of the sterilization load was monitored to ensure that it did not exceed 33° C.

TABLE 2

| Phase | Parameter | Value | |
|---|---|---|---|
| Leak Test | Vacuum Level | 500 | mbar |
| Leak Test | Stabilization Time | 2 | minutes |
| Leak Test | Leak Test Time | 5 | minutes |
| Leak Test | Acceptable Total Leak | 13 | mbar |
| Pre-conditioning | Pulse Quantity | 12 | |
| Pre-conditioning | Vacuum Level | 500 | mbar |
| Pre-conditioning | Vacuum Break Point | 700 | mbar |
| Pre-conditioning | Jacket and Doors | 28° | C. |
| | Temperature | | |
| Pre-conditioning | Vaporizer Temperature | 110° | C. |
| Sterilization | Pulse Quantity | 2 | |
| Sterilization | Vacuum Level | 500 | mbar |
| Sterilization | H₂O₂ (59 wt. % solution) | 150 | g/pulse |
| | Injection Amount | | |
| Sterilization | Injection Cycle Duration | 4500 | milliseconds |
| Sterilization | Post-Injection Hold Time | 2 | minutes |
| Sterilization | Transition Pressure Point | 940 | mbar |
| Sterilization | Post-transition Hold Time | 7 | minutes |

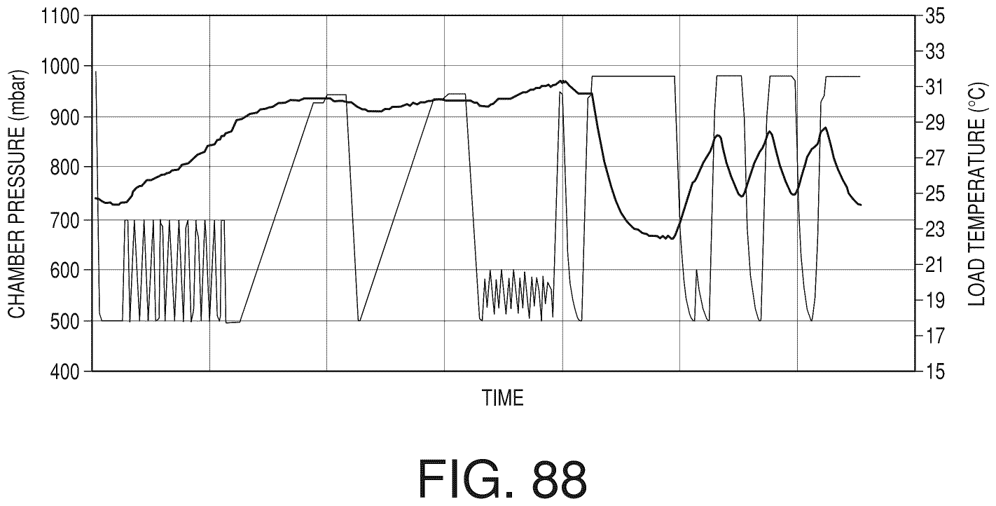

The pressure of the sterilization chamber and the temperature of the load, during the sterilization method of Example 2, were measured and are shown in FIG. 88.

### Example 3

A sterilization load including 24 biological indicators was loaded in a sterilization chamber, and a sterilization method was performed. The sterilization method included a leak test, a preconditioning phase, sterilization phase with two sterilization pulses, and two aeration phases. The exact parameters for the sterilization method are summarized in Table 3.

TABLE 3

| Phase | Parameter | Value | |
|---|---|---|---|
| Leak Test | Vacuum Level | 500 | mbar |
| Leak Test | Stabilization Time | 2 | minutes |
| Leak Test | Leak Test Time | 5 | minutes |
| Leak Test | Acceptable Total Leak | 13 | mbar |
| Pre-conditioning | Pulse Quantity | 12 | |
| Pre-conditioning | Vacuum Level | 500 | mbar |
| Pre-conditioning | Vacuum Break Point | 700 | mbar |
| Pre-conditioning | Jacket and Doors | 28° | C. |
| | Temperature | | |
| Pre-conditioning | Vaporizer Temperature | 110° | C. |
| Sterilization | Pulse Quantity | 2 | |
| Sterilization | Vacuum Level | 500 | mbar |
| Sterilization | H₂O₂ (59 wt. % solution) | 150 | g/pulse |
| | Injection Amount | | |
| Sterilization | Injection Cycle Duration | 4500 | milliseconds |
| Sterilization | Post-Injection Hold Time | 2 | minutes |
| Sterilization | Transition Pressure Point | 940 | mbar |
| Sterilization | Post-transition Hold Time | 7 | minutes |
| First Aeration | Pulse Quantity | 10 | |
| First Aeration | Vacuum Level | 500 | mbar |
| First Aeration | Vacuum Break Point | 899 | mbar |
| First Aeration | Exhaust Time | 0 | minutes |

| | | |
|---|---|---|
| First Aeration | Recirculate During Vacuum | Yes |
| Second Aeration | Pulse Quantity | 10 |
| Second Aeration | Vacuum Level | 500 |
| Second Aeration | Vacuum Break Point | 900 |
| Second Aeration | Exhaust Time | 10 |
| Second Aeration | Recirculate During Vacuum | No |

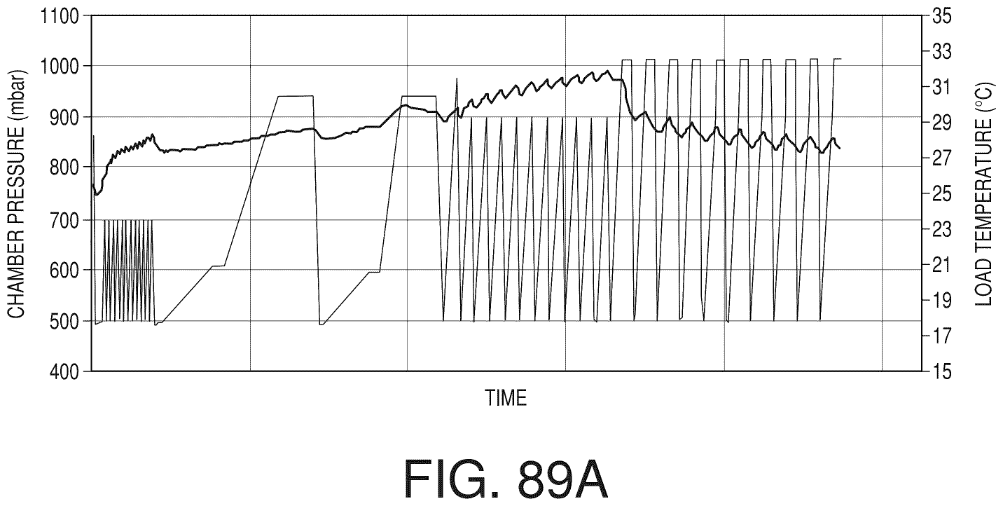

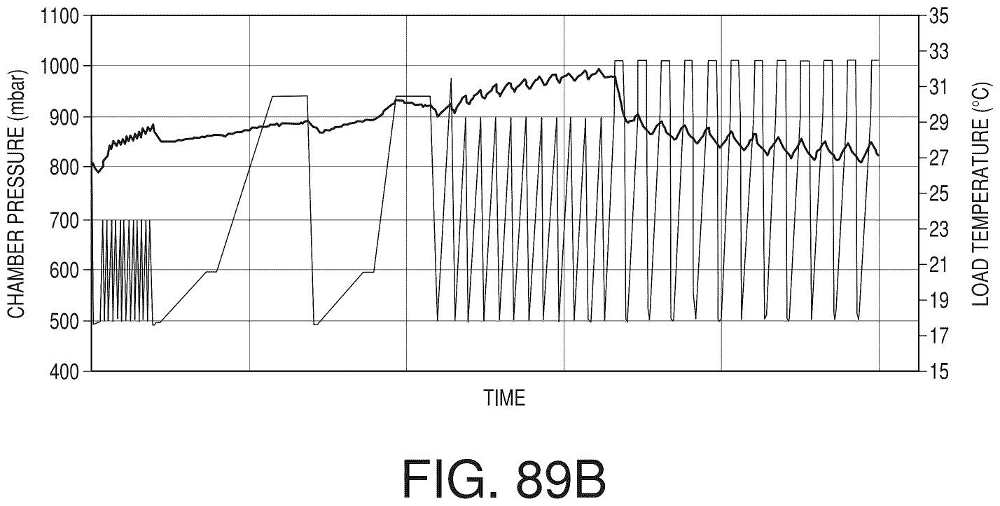

The pressure of the sterilization chamber and the temperature of the load, during the sterilization method of Example 3, were measured and are shown in FIG. 89A.

### Example 4

A sterilization load including 24 biological indicators was loaded in a sterilization chamber, and a sterilization method was performed. The sterilization method included a leak test, a preconditioning phase, sterilization phase with two sterilization pulses, and two aeration phases. The exact parameters for the sterilization method are summarized in Table 4.

TABLE 4

| Phase | Parameter | Value | |
|---|---|---|---|
| Leak Test | Vacuum Level | 500 | mbar |
| Leak Test | Stabilization Time | 2 | minutes |
| Leak Test | Leak Test Time | 5 | minutes |
| Leak Test | Acceptable Total Leak | 13 | mbar |
| Pre-conditioning | Pulse Quantity | 12 | |
| Pre-conditioning | Vacuum Level | 500 | mbar |
| Pre-conditioning | Vacuum Break Point | 700 | mbar |
| Pre-conditioning | Jacket and Doors | 28° | C. |
| | Temperature | | |
| Pre-conditioning | Vaporizer Temperature | 110° | C. |
| Sterilization | Pulse Quantity | 2 | |
| Sterilization | Vacuum Level | 500 | mbar |
| Sterilization | H₂O₂ (59 wt. % solution) | 150 | g/pulse |
| | Injection Amount | | |
| Sterilization | Injection Cycle Duration | 4500 | milliseconds |
| Sterilization | Post-Injection Hold Time | 2 | minutes |
| Sterilization | Transition Pressure Point | 940 | mbar |
| Sterilization | Post-transition Hold Time | 7 | minutes |
| First Aeration | Pulse Quantity | 10 | |
| First Aeration | Vacuum Level | 500 | mbar |
| First Aeration | Vacuum Break Point | 899 | mbar |
| First Aeration | Exhaust Time | 0 | minutes |

| | | |
|---|---|---|
| First Aeration | Recirculate During Vacuum | Yes |
| Second Aeration | Pulse Quantity | 12 |
| Second Aeration | Vacuum Level | 500 |
| Second Aeration | Vacuum Break Point | 900 |
| Second Aeration | Exhaust Time | 10 |
| Second Aeration | Recirculate During Vacuum | No |

The pressure of the sterilization chamber and the temperature of the load, during the sterilization method of Example 4, were measured and are shown in FIG. 89B.

### Example 5

A sterilization load including 24 biological indicators was loaded in a sterilization chamber, and a sterilization method was performed. The sterilization method included a leak test, a preconditioning phase, sterilization phase with three sterilization pulses, and two aeration phases. The exact parameters for the sterilization method are summarized in Table 5.

TABLE 5

| Phase | Parameter | Value | |
|---|---|---|---|
| Leak Test | Vacuum Level | 500 | mbar |
| Leak Test | Stabilization Time | 2 | minutes |
| Leak Test | Leak Test Time | 5 | minutes |
| Leak Test | Acceptable Total Leak | 13 | mbar |
| Pre-conditioning | Pulse Quantity | 12 | |
| Pre-conditioning | Vacuum Level | 500 | mbar |
| Pre-conditioning | Vacuum Break Point | 700 | mbar |
| Pre-conditioning | Jacket and Doors | 28° | C. |
| | Temperature | | |
| Pre-conditioning | Vaporizer Temperature | 110° | C. |
| Sterilization | Pulse Quantity | 3 | |
| Sterilization | Vacuum Level | 500 | mbar |
| Sterilization | H₂O₂ (59 wt. % solution) | 150 | g/pulse |
| | Injection Amount | | |
| Sterilization | Injection Cycle Duration | 4500 | milliseconds |
| Sterilization | Post-Injection Hold Time | 2 | minutes |
| Sterilization | Transition Pressure Point | 940 | mbar |
| Sterilization | Post-transition Hold Time | 7 | minutes |
| First Aeration | Pulse Quantity | 10 | |
| First Aeration | Vacuum Level | 500 | mbar |
| First Aeration | Vacuum Break Point | 899 | mbar |
| First Aeration | Exhaust Time | 0 | minutes |

| | | |
|---|---|---|
| First Aeration | Recirculate During Vacuum | Yes |
| Second Aeration | Pulse Quantity | 9 |
| Second Aeration | Vacuum Level | 500 |
| Second Aeration | Vacuum Break Point | 900 |
| Second Aeration | Exhaust Time | 10 |
| Second Aeration | Recirculate During Vacuum | No |

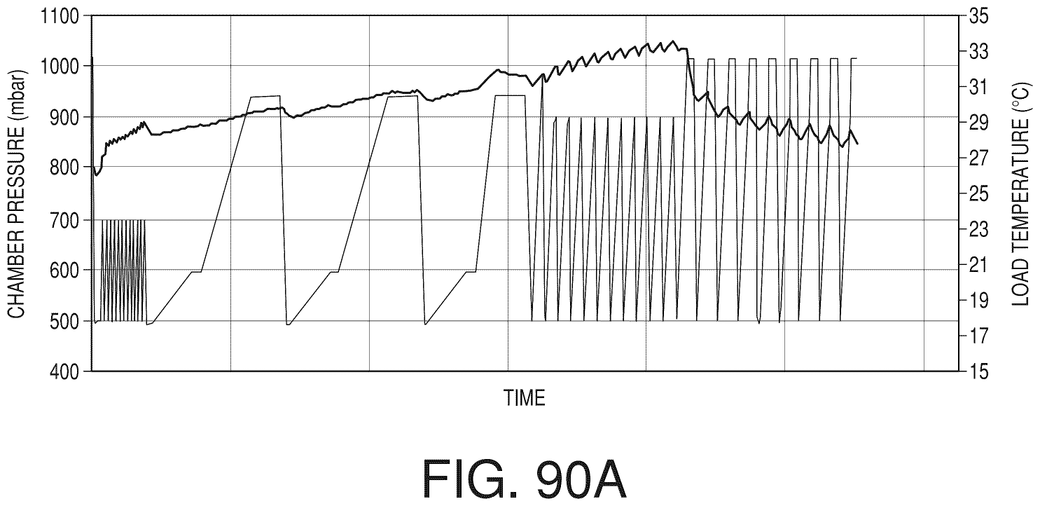

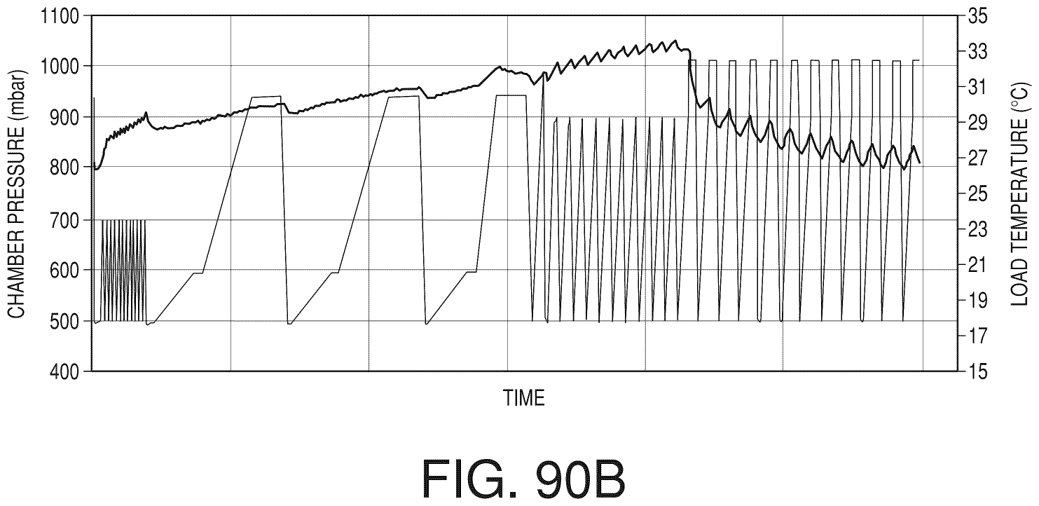

The pressure of the sterilization chamber and the temperature of the load, during the sterilization method of Example 5, were measured and are shown in FIG. 90A.

### Example 6

A sterilization load including 24 biological indicators was loaded in a sterilization chamber, and a sterilization method was performed. The sterilization method included a leak test, a preconditioning phase, sterilization phase with three sterilization pulses, and two aeration phases. The exact parameters for the sterilization method are summarized in Table 6.

TABLE 6

| Phase | Parameter | Value | |
|---|---|---|---|
| Leak Test | Vacuum Level | 500 | mbar |
| Leak Test | Stabilization Time | 2 | minutes |
| Leak Test | Leak Test Time | 5 | minutes |
| Leak Test | Acceptable Total Leak | 13 | mbar |
| Pre-conditioning | Pulse Quantity | 12 | |
| Pre-conditioning | Vacuum Level | 500 | mbar |
| Pre-conditioning | Vacuum Break Point | 700 | mbar |
| Pre-conditioning | Jacket and Doors | 28° | C. |
| | Temperature | | |
| Pre-conditioning | Vaporizer Temperature | 110° | C. |
| Sterilization | Pulse Quantity | 3 | |
| Sterilization | Vacuum Level | 500 | mbar |
| Sterilization | H₂O₂ (59 wt. % solution) | 150 | g/pulse |
| | Injection Amount | | |
| Sterilization | Injection Cycle Duration | 4500 | milliseconds |
| Sterilization | Post-Injection Hold Time | 2 | minutes |
| Sterilization | Transition Pressure Point | 940 | mbar |
| Sterilization | Post-transition Hold Time | 7 | minutes |
| First Aeration | Pulse Quantity | 10 | |
| First Aeration | Vacuum Level | 500 | mbar |
| First Aeration | Vacuum Break Point | 899 | mbar |
| First Aeration | Exhaust Time | 0 | minutes |

| | | |
|---|---|---|
| First Aeration | Recirculate During Vacuum | Yes |
| Second Aeration | Pulse Quantity | 12 |
| Second Aeration | Vacuum Level | 500 |
| Second Aeration | Vacuum Break Point | 900 |
| Second Aeration | Exhaust Time | 10 |
| Second Aeration | Recirculate During Vacuum | No |

The pressure of the sterilization chamber and the temperature of the load, during the sterilization method of Example 6, were measured and are shown in FIG. 90B.

The efficacy and sterilizing efficiency of the sterilization protocols from Examples 1-6 were evaluated with chemical indicators, biological indicators, temperature loggers, humidity loggers, VHP monitors, and a handheld Drager VHP monitoring device. A summary of these results are shown in Table 7.

TABLE 7

| | | | | | Minimum | |
|---|---|---|---|---|---|---|
| | Sterilization | H₂O₂ Used | Max. Load | | Chamber | |
| Sterilization | Time | (g of 57 wt. | Temperature | Total Time | Pressure | BI |
| Method | (hours:minutes) | % solution) | (° C.) | (hours:minutes) | (mbar) | Growth |
| Example 1 | 00:24 | 153.3 | 33.7 | 9:17 | 488 | 24/24 |
| Example 2 | 00:47 | 301.0 | 31.3 | 3:45 | 495 | 1/24 |
| Example 3 | 00:57 | 298.9 | 31.9 | 9:01 | 488 | 0/120 |
| Example 4 | 00:57 | 300.03 | 32.0 | 9:50 | 489 | 0/24 |
| Example 5 | 1:28 | 451.7 | 33.6 | 9:05 | 488 | N/A |
| Example 6 | 1:28 | 451.2 | 33.6 | 10:23 | 488 | 0/24 |

As shown in Table 7, sterilization methods including multiple sterilization pulses prevented biological indicators from growing. Although not shown in Table 7, each exemplary sterilization method also resulted in less than 1.0 part per million of residual VHP. Based on the data observed from the exemplary sterilization methods, it was determined that effective sterilization could be achieved with cycle temperatures less than 35° C., at a vacuum pressure greater than 480 mbar, with at least 300 g of 57 wt. % H₂O₂ solution, and a sterilization time of an hour. This is a more efficient sterilization protocol than previous methodologies, which required at least 500 g of 57 wt. % H₂O₂ solution, and a sterilization time of at least 2 hours and 15 minutes. The more efficient protocol means that more load (e.g., more pre-filled syringes containing medicament) per hour may be sterilized, as compared to methods known in the art.

### Example 7

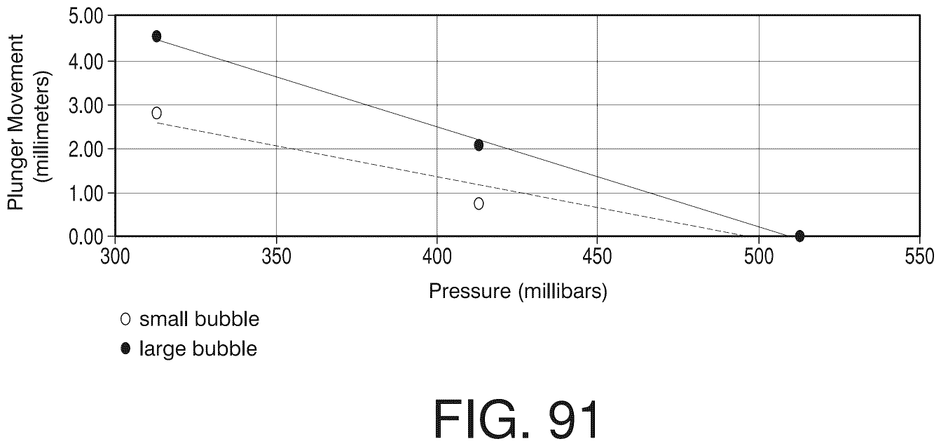

During development of the prefilled syringes, it was determined that the plunger of the prefilled syringe could move up to 2.4 mm before contacting the liquid medicament in the syringe. Syringes with two different air bubble sizes: small (approximately 2 mm diameter) and large (approximately 4 mm diameter) were analyzed at pressures of 313 millibars, 413 millibars, and 513 millibars. The movement of the plunger at each pressure was plotted as a function of the pressure. Linear regression was used to determine a line of best fit for each bubble size, correlating a relationship between plunger movement and pressure. The resulting plot and lines of best fit are shown in FIG. 91. The line of best fit for the small bubble was y=−0.014x+6.938, where x is the pressure in millibars and y is the movement of the plunger in millimeters. The line of best fit for the large bubble was y=−0.0228x+11.602, where x is the pressure in millibars and y is the movement of the plunger in millimeters.

The lines of best fit were used to extrapolate plunger movement at 480 millibars. As shown in FIG. 91, this plunger movement at 480 millibars is about 0.22-0.66 mm, depending on the bubble size. Based on this analysis, a minimum sterilization pressure of 480 millibars was determined to have no impact on the sterile boundary between plunger and liquid medicament.

### Example 8

A sterilization method (Sterilization Method I) was performed on two different sterilization loads, Sterilization Load A and Sterilization Load B, according to the phases and parameters shown in Table 8. Sterilization Method I included two primary sterilant exposures, three secondary sterilant exposures, seven tertiary sterilant exposures, and seven aeration pulses. Each primary sterilant exposure had the same parameters as the other primary sterilant exposure, each secondary sterilant exposure had the same parameters as the other secondary sterilant exposures, and each tertiary sterilant exposure had the same parameters as the other tertiary sterilant exposures. Sterilization Load A included eight carts, with each cart including 1,368 prefilled syringes. Sterilization Load B included eight carts, with each cart including 76 prefilled syringes. Each prefilled syringe of Sterilization Load A and Sterilization Load B included about 0.19 grams of liquid medicament (i.e., about 0.18 mL). Each sterilization load also included 144 biological indicators placed inside blister packages and distributed throughout the sterilization load.

TABLE 8

| | | Value for | | Value for | |
|---|---|---|---|---|---|
| | | Sterilization | | Sterilization | |
| Phase | Parameter | Method I | | Method II | |
| Leak Test | Vacuum Level | 500 | mbar | 500 | mbar |
| Leak Test | Stabilization Time | 2 | minutes | 2 | minutes |
| Leak Test | Leak Test Time | 5 | minutes | 5 | minutes |
| Leak Test | Acceptable Total Leak | 13 | mbar | 13 | mbar |
| Conditioning | Pulse Quantity | 12 | | 12 | |
| Conditioning | Conditioning Pressure | 500 | mbar | 500 | mbar |
| Conditioning | Conditioning Pressure Break Point | 700 | mbar | 700 | mbar |
| Conditioning | Jacket and Doors Temperature | 28° | C. | 28° | C. |
| Conditioning | Vaporizer Temperature | 110° | C. | 110° | C. |
| Primary Sterilant | Number of Sterilization Pulses | 2 | | 2 | |
| Exposure | | | | | |
| Primary Sterilant | Injection Pressure | 500 | mbar | 500 | mbar |
| Exposure | | | | | |
| Primary Sterilant | H₂O₂ (59 wt. % solution) | 150 | g | 150 | g |
| Exposure | Injection Amount | | | | |
| Primary Sterilant | Injection Cycle Duration | 7000 | msec | 7000 | msec |
| Exposure | | | | | |
| Primary Sterilant | Exposure Hold Time | 1 | minute | 1 | minute |
| Exposure | | | | | |
| Primary Sterilant | Transition Pressure | 940 | mbar | 940 | mbar |
| Exposure | | | | | |
| Primary Sterilant | Distribution Hold Time | 4 | minutes | 4 | minutes |
| Exposure | | | | | |
| Secondary | Number of Sterilization Pulses | 3 | | 2 | |
| Sterilant Exposure | | | | | |
| Secondary | Injection Pressure | 500 | mbar | 500 | mbar |
| Sterilant Exposure | | | | | |
| Secondary | H₂O₂ (59 wt. % solution) | 75 | g | 75 | g |
| Sterilant Exposure | Injection Amount | | | | |
| Secondary | Injection Cycle Duration | 7000 | msec | 7000 | msec |
| Sterilant Exposure | | | | | |
| Secondary | Exposure Hold Time | 1 | minute | 1 | minute |
| Sterilant Exposure | | | | | |
| Secondary | Transition Pressure | 940 | mbar | 940 | mbar |
| Sterilant Exposure | | | | | |
| Secondary | Distribution Hold Time | 4 | minutes | 4 | minutes |
| Sterilant Exposure | | | | | |
| Tertiary Sterilant | Number of Sterilization Pulses | 7 | | 6 | |
| Exposure | | | | | |
| Tertiary Sterilant | Injection Pressure | 600 | mbar | 600 | mbar |
| Exposure | | | | | |
| Tertiary Sterilant | H₂O₂ (59 wt. % solution) | 50 | g | 50 | g |
| Exposure | Injection Amount | | | | |
| Tertiary Sterilant | Injection Cycle Duration | 7000 | msec | 7000 | msec |
| Exposure | | | | | |
| Tertiary Sterilant | Exposure Hold Time | 1 | minute | 1 | minute |
| Exposure | | | | | |
| Tertiary Sterilant | Transition Pressure | 900 | mbar | 900 | mbar |
| Exposure | | | | | |
| Tertiary Sterilant | Distribution Hold Time | 10 | minutes | 10 | minutes |
| Exposure | | | | | |
| First Aeration | Vacuum Level | 500 | mbar | 500 | mbar |
| First Aeration | Exhaust Pressure | 940 | mbar | 940 | mbar |
| First Aeration | Exhaust Time | 35 | minutes | 35 | minutes |
| Second Aeration | Vacuum Level | 500 | mbar | 500 | mbar |
| Second Aeration | Exhaust Pressure | 700 | mbar | 700 | mbar |
| Second Aeration | Exhaust Time | 0 | minutes | 0 | minutes |
| Third Aeration | Vacuum Level | 500 | mbar | 500 | mbar |
| Third Aeration | Exhaust Pressure | 940 | mbar | 940 | mbar |
| Third Aeration | Exhaust Time | 15 | minutes | 15 | minutes |
| Fourth Aeration | Vacuum Level | 500 | mbar | 500 | mbar |
| Fourth Aeration | Exhaust Pressure | 940 | mbar | 940 | mbar |
| Fourth Aeration | Exhaust Time | 15 | minutes | 15 | minutes |
| Fifth Aeration | Vacuum Level | 500 | mbar | 500 | mbar |
| Fifth Aeration | Exhaust Pressure | 940 | mbar | 940 | mbar |
| Fifth Aeration | Exhaust Time | 15 | minutes | 15 | minutes |
| Sixth Aeration | Vacuum Level | 500 | mbar | 500 | mbar |
| Sixth Aeration | Exhaust Pressure | 600 | mbar | 600 | mbar |
| Sixth Aeration | Exhaust Time | 0 | minutes | 0 | minutes |
| Seventh Aeration | Vacuum Level | 900 | mbar | 900 | mbar |
| Seventh Aeration | Exhaust Pressure | 940 | mbar | 940 | mbar |
| Seventh Aeration | Exhaust Time | 15 | minutes | 15 | minutes |

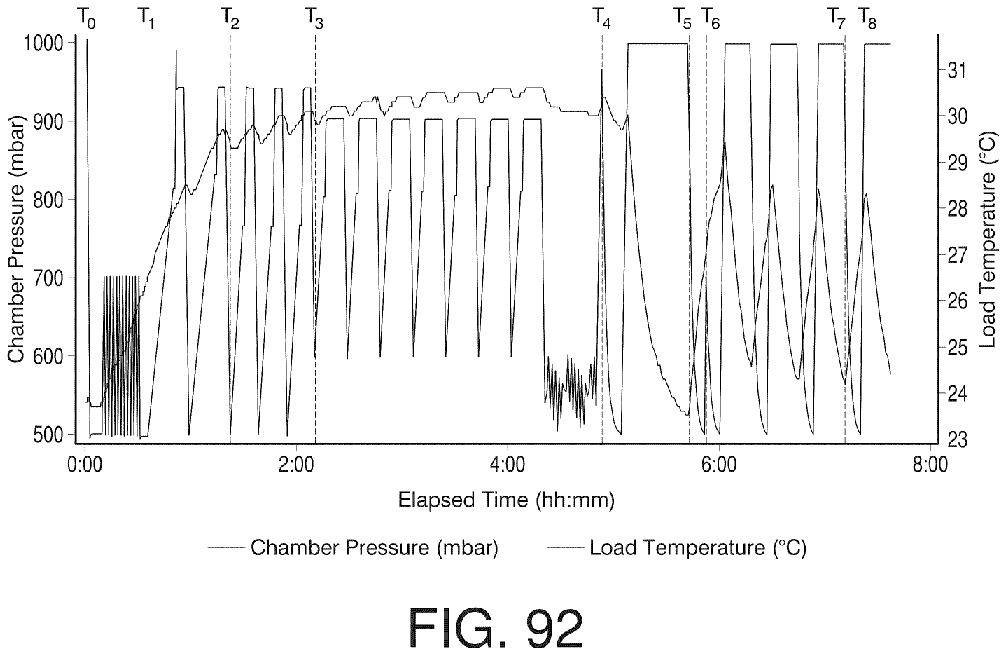

Referring to FIG. 92, the pressure and temperature within the sterilization chamber were plotted as a function of time elapsed, during the performance of Sterilization Method I. The leak test and conditioning steps were performed from time T0 to time T1. The primary sterilant exposures were performed from time T1 to time T2, the secondary sterilant exposures were performed from time T2 to time T3, and the tertiary sterilant exposures were performed from time T3 to time T4. The first aeration was performed from time T4 to time T5, the second aeration was performed from time T5 to time T6, the third, fourth, and fifth aerations were performed from time T6 to time T7, the sixth aeration was performed from time T7 to time T8, and the seventh aeration was performed from time T8 to the end of the sterilization method.

After Sterilization Method I was performed, the biological and chemical indicators of Sterilization Load A and Sterilization Load B were analyzed. All biological indicators showed negative growth. Additionally, samples of blister packs were taken from Sterilization Load A and Sterilization Load B, analysis of the sample blister packs demonstrated a six-log reduction in the sterility assurance level.

### Example 9

A sterilization method (Sterilization Method II) was performed on two different sterilization loads, Sterilization Load C and Sterilization Load D, according to the phases and parameters shown in Table 8. Sterilization Method II included two primary sterilant exposures, two secondary sterilant exposures, six tertiary sterilant exposures, and seven aeration pulses. Each primary sterilant exposure had the same parameters as the other primary sterilant exposure, each secondary sterilant exposure had the same parameters as the other secondary sterilant exposure, and each tertiary sterilant exposure had the same parameters as the other tertiary sterilant exposures. Sterilization Load C included eight carts, with each cart including 1,368 prefilled syringes. Sterilization Load D included eight carts, with each cart including 76 prefilled syringes. Each prefilled syringe of Sterilization Load C and Sterilization Load D included about 0.19 grams of liquid medicament (i.e., about 0.18 mL). Each sterilization load also included 30 biological indicators distributed throughout the sterilization load.

After Sterilization Method II was performed, the biological and chemical indicators of Sterilization Load C and Sterilization Load D were analyzed. All biological indicators showed negative growth. Additionally, samples of blister packs were taken from Sterilization Load C and Sterilization Load D, analysis of the sample blister packs demonstrated a six-log reduction in the sterility assurance level.

Sterilization Method II included less secondary sterilant exposures and less tertiary sterilant exposures than Sterilization Method I. As a result, Sterilization Method I included 125 g more of total sterilant exposure than Sterilization Method II. Accordingly, Sterilization II would be expected to be less effective than Sterilization Method I. All biological indicators showing negative growth with Sterilization Method II further evidences that Sterilization Method I is effective at sterilizing the sterilization loads.

### Example 10

After Sterilization Loads A and B were sterilized according to Sterilization Method I, the sterilized prefilled syringes were stored at 25° C. for 6 months. At the time of storage, at 1 month of storage, at 3 months of storage, and at 6 months of storage, the product quality of the prefilled syringes was evaluated to determine if the sterilization method or storage process had an adverse effect on the prefilled syringes. The results of these product quality analyses are summarized in Table 9.

TABLE 9

| | | Results | | | |
|---|---|---|---|---|---|
| | End-of-Shelf-Life | Length of Storage (months) | | | |
| Assay | Acceptance Criteria | t = 0 | 1 | 3 | 6 |
| Appearance | Liquid essentially free | Pass | Pass | Pass | Pass |
| | from visible particulates | | | | |
| Clarity | Not more turbid than | Pass | Pass | Pass | Pass |
| (Turbidity) | Reference Suspension | | | | |
| Color | Not more intensely | Pass | Pass | Pass | Pass |
| | colored than Reference | | | | |
| | Solution | | | | |
| pH | 5.6-6.1 | 5.9 | 5.9 | 5.9 | 5.9 |
| Total Protein | 103.0-126.0 | 113.0 | 114.4 | 113.6 | 115.2 |
| Content (A₂₈₀) | mg/mL | | | | |
| Potency by | 70%-130% of reference | 113 | 86 | 91 | 79 |
| Bioassay | standard | | | | |
| Purity by | a. ≥91.5% purity | 95.1 | 94.1 | 92.9 | 91.1 |
| Reduced CE-SDS | b. ≤2.5% | 1.2 | 1.6 | 2.1 | 2.6 |
| a. % Purity | c. ≤6.0% | 3.7 | 4.3 | 4.9 | 6.3 |
| b. % R1 | | | | | |
| c. % LMW less | | | | | |
| R1 | | | | | |
| Purity by Non- | main peak ≥96.5% total | 99.3 | 98.5 | 98.2 | 96.9 |
| Reduced CE- | area | | | | |
| SDS | | | | | |
| % Main peak | | | | | |
| purity | | | | | |
| Purity by SE-UPLC | a. main peak ≥94.0% | 98.0 | 96.3 | 94.5 | 91.9 |
| a. % Main Peak | total area | | | | |
| b. % Aggregate | b. ≤5.0% aggregate | 2.0 | 3.4 | 5.0 | 7.4 |
| Charged | The test article profile | Pass | Pass | Pass | Pass |
| Variant | should be qualitatively | | | | |
| Analysis by | similar to the Reference | | | | |
| iCIEF | Standard | | | | |
| a. % Region 1 | electropherogram in terms | | | | |
| b. % Region 2 | of intensity, number, and | | | | |
| c. % Region 3 | pattern of peaks. | | | | |
| | a. 21.5-38.4% | 29 | 29 | 30 | 30 |
| | b. 38.5-50.4% | 45 | 45 | 45 | 46 |
| | c. 13.5-36.4% | 25 | 26 | 0.26 | 0.41 |
| Isoaspartate | ≤0.22 mol | 0.08 | 0.16 | 0.26 | 0.41 |
| | isoaspartate/mol target | | | | |
| | protein | | | | |
| Polysorbate 20 | 0.022%-0.038% (w/v) | | 0.030 | 0.028 | 0.032 |
| Particulate | ≤50 particles/mL | 5 | 5 | 1 | 1 |
| Matter | (≥10 μm) | | | | |
| (microscopic) | ≤5 particles/mL | 2 | 1 | 0 | 1 |
| | (≥25 μm) | | | | |
| | ≤2 particles/mL | 0 | 0 | 0 | 0 |
| | (≥50 μm) | | | | |
| Maximum | ≤28.2 Newtons (N) | | | | 15.0 |
| Injection Force | | | | | |
| Deliverable | 0.07 mL ≤ X < 0.10 mL | | | | 0.086 |
| Volume | | | | | |
| Maximum Use | ≤13 N · cm | | | | ≤13 N · cm |
| Torque | | | | | |

The data from these product quality analyses demonstrate that the sterilization method can achieve sterility acceptance criteria without adverse impact to the drug product.

Terminal sterilization of packaged medical products (e.g., pre-filled delivery devices) may require sterilizing agents to traverse several layers of packaging materials, container materials, and/or labels, to effectively sterilize all aspects of a load, and to be appropriately removed from all aspects of the load. In some cases, packaging may include semi-permeable materials, which are permeable for sterilizing agents in a particular phase (e.g., vapor). For example, packaging may include a blister pack comprising a semi-permeable material, and plurality of medical products, prefilled with a liquid medicament, disposed in the blister pack.

Packaging for medical products may resist penetration of sterilization materials, and/or may be sensitive to temperature and pressure changes caused by sterilization. For example, medical products may be packaged in a plastic ‘blister’ configured to house the syringe and restrict it from movement. Such a blister may be only somewhat permeable to sterilization materials, and/or may be sensitive to changes in pressure.

In the case of some types of sterilization, such as terminal sterilization, sterilizing agents may need to traverse one or more layers of packaging, to sterilize the exterior of each medical product as well as the interior of packaging elements. Sterilizing agents may also need to be successfully removed through one or more layers of packaging, to avoid remaining as residue on a medical product.

As described herein, the characteristics of a sterilization load may affect distribution and movement of vaporized sterilant during a sterilization process. As a result, conventional sterilization processes may be ineffective for sterilization loads including some arrangements of packaged drug delivery devices. For example, sterilization loads including packaged delivery devices pre-filled with a liquid medicament may be insufficiently sterilized during conventional sterilization methods. Characteristics of a sterilization load that may affect distribution and movement of vaporized sterilant during a sterilization process include a mass of the sterilization load, a ratio of a mass of the liquid medicament in the load to a total mass of the load, a volume of the delivery device, a volume of the packaging, one or more dimensions of the packaging, a material composition of the medicament, a material composition of the delivery device, and/or a material composition of the packaging.

Methods of the present disclosure may be particularly effective when used with sterilization loads having particular, weight, volume, and/or other physical characteristics. Such sterilization loads may include a greater amount of liquid compared to conventional sterilization loads. For example, methods of the present disclosure may be particularly effective when used with sterilization loads that include a greater mass of liquid relative to a total mass of the sterilization load, compared to conventional sterilization loads. Sterilization loads for which the methods of the present disclosure are particularly effective may be referred to as liquid heavy sterilization loads.

A liquid heavy sterilization load may include one or more packages (e.g., one or more blister packs), where each package includes one or more pre-filled delivery devices. Each package in a liquid heavy sterilization load may include at least about 0.15 grams of liquid mass, such as, for example, at least about 0.17 grams, at least about 0.18 grams, at least about 0.19 grams, at least about 0.20 grams, at least about 0.21 grams, at least about 0.22 grams, at least about 0.23 grams, at least about 0.24 grams, at least about 0.25 grams, about 0.15 grams to about 0.25 grams, about 0.17 grams to about 0.25 grams, about 0.19 grams to about 0.25 grams, about 0.21 grams to about 0.25 grams, about 0.15 grams to about 0.23 grams, about 0.17 grams to about 0.23 grams, about 0.19 grams to about 0.23 grams, about 0.21 grams to about 0.23 grams, about 0.15 grams to about 0.21 grams, about 0.17 grams to about 0.21 grams, about 0.19 grams to about 0.21 grams, about 0.15 grams to about 0.19 grams, about 0.17 grams to about 0.19 grams, or about 0.15 grams to about 0.17 grams.

A liquid heavy sterilization load may include at least about 500 pre-filled delivery devices, such as, for example, at least 550 pre-filled delivery devices, at least 600 pre-filled delivery devices, at least 650 pre-filled delivery devices, at least 700 pre-filled delivery devices, at least 750 pre-filled delivery devices, at least 800 pre-filled delivery devices, at least 850 pre-filled delivery devices, at least 900 pre-filled delivery devices, at least 1000 pre-filled delivery devices, at least 1500 pre-filled delivery devices, at least 2000 pre-filled delivery devices, at least 3000 pre-filled delivery devices, at least 4000 pre-filled delivery devices, at least 5000 pre-filled delivery devices, at least 6000 pre-filled delivery devices, at least 7000 pre-filled delivery devices, at least 8000 pre-filled delivery devices, at least 9000 pre-filled delivery devices, at least 10,000 pre-filled delivery devices, at least 11,000 pre-filled delivery devices, at least 12,000 pre-filled delivery devices, at least 13,000 pre-filled delivery devices, at least 14,000 pre-filled delivery devices, or at least 15,000 pre-filled delivery devices.

A liquid heavy sterilization load may include a total liquid mass of at least about 100 grams, such as, for example, at least about 105 grams, at least about 115 grams, at least about 125 grams, at least about 135 grams, at least about 145 grams, at least about 150 grams, at least about 1750 grams, at least about 1800 grams, at least about 1850 grams, at least about 1900 grams, at least about 1950 grams, 2000 grams, at least about 2050 grams, at least about 2100 grams, at least about 2150 grams, at least about 2200 grams, at least about 2250 grams, at least about 2300 grams, at least about 2350 grams, at least about 2450 grams, at least about 2500 grams, at least about 2550 grams, 100 grams to about 150 grams, about 105 grams to about 150 grams, about 110 grams to about 150 grams, about 115 grams to about 150 grams, about 120 grams to about 150 grams, about 125 grams to about 150 grams, about 130 grams to about 150 grams, about 135 grams to about 150 grams, about 140 grams to about 150 grams, about 145 grams to about 150 grams, about 100 grams to about 145 grams, about 105 grams to about 145 grams, about 110 grams to about 145 grams, about 115 grams to about 145 grams, about 120 grams to about 145 grams, about 125 grams to about 145 grams, about 130 grams to about 145 grams, about 135 grams to about 145 grams, about 140 grams to about 145 grams, about 100 grams to about 140 grams, about 105 grams to about 140 grams, about 110 grams to about 140 grams, about 115 grams to about 140 grams, about 120 grams to about 140 grams, about 125 grams to about 140 grams, about 130 grams to about 140 grams, about 135 grams to about 140 grams, about 100 grams to about 135 grams, about 105 grams to about 135 grams, about 110 grams to about 135 grams, about 115 grams to about 135 grams, about 120 grams to about 135 grams, about 125 grams to about 135 grams, about 130 grams to about 135 grams, about 100 grams to about 130 grams, about 105 grams to about 130 grams, about 110 grams to about 130 grams, about 115 grams to about 130 grams, about 120 grams to about 130 grams, about 125 grams to about 130 grams, about 100 grams to about 125 grams, about 105 grams to about 125 grams, about 110 grams to about 125 grams, about 115 grams to about 125 grams, about 120 grams to about 125 grams, about 100 grams to about 120 grams, about 105 grams to about 120 grams, about 110 grams to about 120 grams, about 115 grams to about 120 grams, about 100 grams to about 115 grams, about 105 grams to about 115 grams, about 110 grams to about 115 grams, about 100 grams to about 110 grams, about 105 grams to about 110 grams, about 100 grams to about 105 grams, about 1750 grams to about 2550 grams, about 1850 grams to about 2550 grams, about 1950 grams to about 2550 grams, about 2050 grams to about 2550 grams, about 2150 grams to about 2550 grams, about 2250 grams to about 2550 grams, about 2350 grams to about 2550 grams, about 2450 grams to about 2550 grams, about 1750 grams to about 2450 grams, about 1850 grams to about 2450 grams, about 1950 grams to about 2450 grams, about 2050 grams to about 2450 grams, about 2150 grams to about 2450 grams, about 2250 grams to about 2450 grams, about 2350 grams to about 2450 grams, about 1750 grams to about 2350 grams, about 1850 grams to about 2350 grams, about 1950 grams to about 2350 grams, about 2050 grams to about 2350 grams, about 2150 grams to about 2350 grams, about 2250 grams to about 2350 grams, about 1750 grams to about 2250 grams, about 1850 grams to about 2250 grams, about 1950 grams to about 2250 grams, about 2050 grams to about 2250 grams, about 2150 grams to about 2250 grams, about 1750 grams to about 2150 grams, about 1850 grams to about 2150 grams, about 1950 grams to about 2150 grams, about 2050 grams to about 2150 grams, about 1750 grams to about 2050 grams, about 1850 grams to about 2050 grams, about 1950 grams to about 2050 grams, about 1750 grams to about 1950 grams, about 1850 grams to about 1950 grams, or about 1750 grams to about 1850 grams.

Each pre-filled delivery device of a liquid heavy sterilization load may include a total interior volume of at least about 0.3 mL, such as, for example, at least about 0.4 mL, at least about 0.5 mL, at least about 0.6 mL, at least about 0.7 mL, at least about 0.8 mL, at least about 0.9 mL, about 0.3 mL, about 0.4 mL, about 0.5 mL about 0.6 mL, about 0.7 mL, about 0.8 mL, or about 0.9 mL.

A liquid heavy sterilization load may include at least about 0.15 mL of liquid per pre-filled delivery device, such as, for example, at least about 0.17 mL, at least about 0.18 mL, at least about 0.19 mL, at least about 0.20 mL, at least about 0.21 mL, at least about 0.22 mL, at least about 0.23 mL, at least about 0.24 mL, at least about 0.25 mL, about 0.15 mL to about 0.25 mL, about 0.17 mL to about 0.25 mL, about 0.19 mL to about 0.25 mL, about 0.21 mL to about 0.25 mL, about 0.15 mL to about 0.23 mL, about 0.17 mL to about 0.23 mL, about 0.19 mL to about 0.23 mL, about 0.21 mL to about 0.23 mL, about 0.15 mL to about 0.21 mL, about 0.17 mL to about 0.21 mL, about 0.19 mL to about 0.21 mL, about 0.15 mL to about 0.19 mL, about 0.17 mL to about 0.19 mL, or about 0.15 mL to about 0.17 mL.

After each delivery device of the liquid heavy sterilization load is pre-filled, the delivery device may include an inert or nonreactive gas such as, air, nitrogen, or another inert or nonreactive gas. The inert or nonreactive gas in the pre-filled delivery device may be referred to as a bubble, a space, a gap, and/or an air pocket. The pre-filled delivery device may include a bubble having a volume of about 0.02 mL to about 0.1 mL, such as, for example, about 0.02 mL, about 0.03 mL, about 0.04 mL, about 0.05 mL, about 0.06 mL, about 0.07 mL, about 0.08 mL, about 0.09 mL, or about 0.1 mL.

A liquid heavy sterilization load may include at least 100 mL of liquid, such as, for example, at least about 105 mL, at least about 115 mL, at least about 125 mL, at least about 135 mL, at least about 145 mL, at least about 150 mL, at least about 1750 mL, at least about 1800 mL, at least about 1850 mL, at least about 1900 mL, at least about 1950 mL, 2000 mL, at least about 2050 mL, at least about 2100 mL, at least about 2150 mL, at least about 2200 mL, at least about 2250 mL, at least about 2300 mL, at least about 2350 mL, at least about 2450 mL, at least about 2500 mL, at least about 2550 mL, 100 mL to about 150 mL, about 105 mL to about 150 mL, about 110 mL to about 150 mL, about 115 mL to about 150 mL, about 120 mL to about 150 mL, about 125 mL to about 150 mL, about 130 mL to about 150 mL, about 135 mL to about 150 mL, about 140 mL to about 150 mL, about 145 mL to about 150 mL, about 100 mL to about 145 mL, about 105 mL to about 145 mL, about 110 mL to about 145 mL, about 115 mL to about 145 mL, about 120 mL to about 145 mL, about 125 mL to about 145 mL, about 130 mL to about 145 mL, about 135 mL to about 145 mL, about 140 mL to about 145 mL, about 100 mL to about 140 mL, about 105 mL to about 140 mL, about 110 mL to about 140 mL, about 115 mL to about 140 mL, about 120 mL to about 140 mL, about 125 mL to about 140 mL, about 130 mL to about 140 mL, about 135 mL to about 140 mL, about 100 mL to about 135 mL, about 105 mL to about 135 mL, about 110 mL to about 135 mL, about 115 mL to about 135 mL, about 120 mL to about 135 mL, about 125 mL to about 135 mL, about 130 mL to about 135 mL, about 100 mL to about 130 mL, about 105 mL to about 130 mL, about 110 mL to about 130 mL, about 115 mL to about 130 mL, about 120 mL to about 130 mL, about 125 mL to about 130 mL, about 100 mL to about 125 mL, about 105 mL to about 125 mL, about 110 mL to about 125 mL, about 115 mL to about 125 mL, about 120 mL to about 125 mL, about 100 mL to about 120 mL, about 105 mL to about 120 mL, about 110 mL to about 120 mL, about 115 mL to about 120 mL, about 100 mL to about 115 mL, about 105 mL to about 115 mL, about 110 mL to about 115 mL, about 100 mL to about 110 mL, about 105 mL to about 110 mL, about 100 mL to about 105 mL, about 1750 mL to about 2550 mL, about 1850 mL to about 2550 mL, about 1950 mL to about 2550 mL, about 2050 mL to about 2550 mL, about 2150 mL to about 2550 mL, about 2250 mL to about 2550 mL, about 2350 mL to about 2550 mL, about 2450 mL to about 2550 mL, about 1750 mL to about 2450 mL, about 1850 mL to about 2450 mL, about 1950 mL to about 2450 mL, about 2050 mL to about 2450 mL, about 2150 mL to about 2450 mL, about 2250 mL to about 2450 mL, about 2350 mL to about 2450 mL, about 1750 mL to about 2350 mL, about 1850 mL to about 2350 mL, about 1950 mL to about 2350 mL, about 2050 mL to about 2350 mL, about 2150 mL to about 2350 mL, about 2250 mL to about 2350 mL, about 1750 mL to about 2250 mL, about 1850 mL to about 2250 mL, about 1950 mL to about 2250 mL, about 2050 mL to about 2250 mL, about 2150 mL to about 2250 mL, about 1750 mL to about 2150 mL, about 1850 mL to about 2150 mL, about 1950 mL to about 2150 mL, about 2050 mL to about 2150 mL, about 1750 mL to about 2050 mL, about 1850 mL to about 2050 mL, about 1950 mL to about 2050 mL, about 1750 mL to about 1950 mL, about 1850 mL to about 1950 mL, or about 1750 mL to about 1850 mL.

Each package in a liquid heavy sterilization load may have a length of about 135 mm to about 145 mm, such as, for example, about 135 mm, about 136 mm, about 137 mm, about 138 mm, about 139 mm, about 140 mm, about 141 mm, about 142 mm, about 143 mm, about 144 mm, or about 145 mm.

Each package in a liquid heavy sterilization load may have a width of about 70 mm to about 85 mm, about 70 mm to about 80 mm, about 70 mm to about 75 mm, about 75 mm to about 85 mm, about 75 mm to about 85 mm, or about 75 mm to about 80 mm, such as, for example, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, or about 85 mm.

Each package in a liquid heavy sterilization load may have a height of about 20 mm to about 30 mm, about 20 mm to about 25 mm, or about 25 mm to about 30 mm, such as, for example, about 20 mm, about 21 mm, about 22 mm, about 23 mm, about 24 mm, about 25 mm, about 26 mm, about 27 mm, about 28 mm, about 29 mm, or about 30 mm.

As described herein, a package within a sterilization load may comprise a flashspun high-density semi-permeable polyethylene polymer. In a liquid heavy sterilization load, the flashspun high-density semi-permeable polyethylene polymer may have a thickness of about 0.2 mm to about 1.5 mm, about 0.5 mm to about 1.5 mm, or about 1.0 to about 1.5 mm, such as, for example, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, or about 1.5 mm.

Each package in a liquid heavy sterilization load may have a volume of about 250 mL to about 300 mL, about 275 mL to about 300 mL, or about 250 mL to about 275 mL, such as for example, about 250 mL, about 255 mL, about 260 mL, about 265 mL, about 270 mL, about 275 mL, about 280 mL, about 285 mL, about 290 mL, about 295 mL, or about 300 mL.

A liquid heavy sterilization load may have a volume of about 155 L to about 3200 L, about 155 L to about 170 L, or about 2900 L to about 3000 L, such as, for example, about 155 L, about 160 L, about 165 L, about 170 L, about 2900 L, about 2950 L, about 3000 L, about 3050 L, about 3150 L, or about 3200 L.

A liquid heavy sterilization load may include a ratio of liquid mass to total load mass of about 0.005 to about 0.02, or about 0.011 to about 0.013, such as, for example, about 0.005, about 0.006, about 0.007, about 0.008, about 0.009, about 0.01, about 0.011, about 0.012, about 0.013, about 0.014, about 0.015, about 0.016, about 0.017, about 0.018, about 0.019, or about 0.2.

A liquid heavy sterilization load may include a ratio of liquid volume to total load volume of about 0.0005 to about 0.001, or about 0.0006 to about 0.00075, such as, for example, about 0.0005, about 0.00055, about 0.0006, about 0.00065, about 0.0007, about 0.00075, about 0.0008, about 0.00085, about 0.0009, about 0.00095, or about 0.001.

During a sterilization process, medical products may be located within different portions of a sterilization chamber. Throughout the sterilization process, each portion of the sterilization chamber may exhibit conditions that may differ from those in other parts of the chamber, such as temperature, pressure, water vapor concentration, humidity, or sterilant concentration. The differing conditions may affect sterilization efficacy. Maintenance of a consistent environment throughout a sterilization chamber may be beneficial to ensuring sterilization efficacy is adequate as to all parts of a load.

An environment within a sterilization chamber may change during a sterilization cycle, such as in a way that affects the movement, state, or efficacy of sterilant and/or fluids in the chamber. For example, as sterilant is added to a chamber, a pressure within the chamber may increase. The pressure within the chamber may affect the ratio of condensed sterilant to vaporized sterilant. Changes in temperature, humidity, or other environmental characteristics may also affect how sterilant within the chamber and additional sterilant added to the chamber behaves. Sterilization systems and methods that adapt to changes in the environment or climate within the sterilization chamber during a sterilization phase, to maximize efficacy of sterilization, may be beneficial. Systems and methods that adapt to changes in the environment or climate of an area to maximize aeration and drying of that area may also be beneficial.

Using a combination of a vaporized chemical sterilant (e.g., VHP) and vaporized water in an environment in which temperature and pressure may be precisely controlled may allow for specific management of the environment to maximize the sterilant's contact with a sterilization load during a sterilization phase, and/or to maximize removal of the sterilant from a load during one or more subsequent aeration or drying phases. Some embodiments of the present disclosure are related to precisely controlling one or more of temperature, pressure, humidity, exposure times, and other environmental conditions. Environmental conditions may be adjusted in any portion of the sterilizing apparatus, before, during, and/or after a sterilization process is performed with the apparatus. For example, the environment of one or more portions of the apparatus where sterilant is introduced or removed, may be maintained or controlled to be within pre-determined conditions. As such, embodiments of the present disclosure may aid in improving the introduction and/or removal of a chemical sterilant in a sterilization apparatus (e.g., between the sterilization apparatus and an exterior of the apparatus, or between portions of the apparatus).

For example, embodiments of the present disclosure may allow for improved infiltration of sterilant through one or more layers of a medical device and/or packaging, compared to conventional methods. In addition or alternatively, embodiments of the present disclosure may allow for improved exfiltration of sterilant through one or more layers of medical device and/or packaging, compared to conventional methods and systems. The improved infiltration of sterilant and/or improved exfiltration of sterilant described herein may be more pronounced when the methods and systems disclosed herein are used with liquid heavy sterilization loads.

Several characteristics of a vaporized chemical sterilant may (positively or negatively) affect the safety, efficacy, efficiency, and other aspects of sterilization processes for medical products. Chemical sterilant vapors and water vapors in an environment may adsorb to, and/or condense on, surfaces having relatively cooler temperatures within the sterilization chamber. For example, during vapor sterilization of sterilization loads including pre-filled delivery devices or medical produces, “cold spots” created by liquid medicament (e.g., a liquid with a high heat capacity), may attract vapor adsorption and/or promote surface condensation. The relative size and mass of the “cold spots” of liquid medicament may affect how chemical sterilant and water vapors adsorb on surfaces, condense on surfaces, or traverse surfaces (e.g., permeable packaging layers). Parameters of a sterilization method may be adjusted, depending on the relative sizes and distributions of liquid medicaments within the sterilization load, in order to promote improved transportation of the sterilant through the sterilization load.

Adjustments in the temperature of a sterilization chamber may generate areas of relative warmth and coolness within the environment, which may in turn affect the relative temperature of a sterilization load in an area of relative warmth or coolness. For example, heating a sterilization chamber using a temperature control jacket may result in areas of the chamber closest to the jacket (e.g., a periphery of the chamber) becoming warmer than areas farther from the jacket (e.g., a middle of the chamber). The ambient heat in warmer areas may cause parts of a sterilization load in those areas to become relatively warmer as well. Chemical sterilant vapors and water vapors may preferentially adsorb to the surfaces of areas having relatively cooler temperatures as compared to the rest of the environment (“cold spots”); thus, vaporized chemical sterilant (e.g., VHP) may not distribute evenly between areas of relative warmth and coolness. While cooler areas may be subject to more thorough exposure to sterilant, warmer areas may experience more thorough aeration and drying.

VHP may preferentially adsorb onto surfaces as compared to water vapor, due to the fact that hydrogen peroxide is more dense and less volatile than water. In some instances, hydrogen peroxide and water vapor may be adsorbing and condensing on surfaces at the same time, with hydrogen peroxide adsorbing and condensing in greater quantities and percentages as compared to the water vapor, and in closer proximity to the surfaces of the sterilization load than the water vapor. Multiple layers of adsorption may form on a single surface in a sterilization environment. In some instances, each layer of adsorption and/or condensation further away from the surface may contain less hydrogen peroxide and more water vapor, such that a gradient of hydrogen peroxide to water is formed on the surface. Hydrogen peroxide may preferentially adsorb and condense closer to the surface than water because of the thermodynamic behavior of binary mixtures of VHP and water vapor near or at saturation (e.g., a binary mixture of hydrogen peroxide and water at vapor/liquid equilibrium). Vapor/liquid equilibrium may be analogous to gas/adsorbate equilibrium for binary mixtures of VHP and water vapor in sterilization applications.

In some instances, condensed or adsorbed hydrogen peroxide may be difficult to remove from surfaces. For example, condensation of water vapor over the condensed/adsorbed hydrogen peroxide, or adsorption of water around the condensed/adsorbed hydrogen peroxide, may trap the hydrogen peroxide on the sterilized surface, or otherwise inhibit the removal of the hydrogen peroxide. This effect may be more pronounced in sterilization loads having relatively higher masses or volumes of liquid medicament (e.g., a higher ratio of mass of liquid medicament to mass of sterilization load).

Differences in pressure throughout an environment, such as a sterilization chamber, may also affect efficacy of vaporized chemical sterilant. For example, sterilization efficacy may be greater at compressed air injection points of a sterilization chamber, compared to the rest of the chamber. Without being limited by theory, this may be due to the nature of gas within the chamber at a partial vacuum. Areas local to a compressed air injection point may experience a pressure wave or pulse having greater magnitude than areas farther from a compressed air injection point while the chamber is saturated with chemical sterilant. The pressure wave may cause greater condensation of chemical sterilant in areas local to compressed air injection points. The characteristics and movement of the pressure wave may be affected by characteristics of the sterilization load. For example, sterilization loads including relatively higher masses or volumes of liquid medicament may impede or dampen pressure waves generated in the sterilization chamber, compared to sterilization loads with lower masses and volumes of liquid medicament.

In some cases where it is desired that a sterilant traverse a semi-permeable membrane of a load to sterilize an interior area or volume covered by the membrane, a delay in migration of at least a portion of sterilant through the load has been observed. For example, in sterilization loads including a semi-permeable Tyvek® membrane, the hydrogen peroxide concentration within the membrane lagged, or was slower to equilibrate with, the hydrogen peroxide concentration outside of the membrane. No such delay or lag was observed with respect to water concentration. Thus, the relative strength of a sterilant may be lower for loads or portions of a load within a semi-permeable membrane (either for part or for all of a sterilization cycle) as compared to the strength of the sterilant outside of the membrane. This effect may be more pronounced in sterilization loads having relatively higher masses or volumes of liquid medicament (e.g., a higher ratio of mass of liquid medicament to mass of sterilization load). Accordingly, parameters of sterilization methods for such loads may need to be adjusted in order to ensure proper aeration, drying, and/or removal of sterilant.

The speed with which pressure increases during introduction of vaporized sterilant into a sterilization chamber may negatively affect sterilization efficacy. A pressure increase to some degree may benefit introduction of sterilant to a load, promoting adsorption of the sterilant to the load. However, excessive pressure increases when an environment is at or near a level of VHP saturation, for example, may result in aggressive condensation of the VHP, which may compromise sterilization efficacy or subsequent aeration or drying efficacy. Allowing the pressure of an environment to be held at a level where vaporized sterilant may condense over time may result in over-condensation of vaporized sterilant. Conversely, decreasing the pressure of an environment after introducing vaporized sterilant (e.g., VHP) may allow more sterilant to remain in a vapor phase, which may improve the sterilant's efficacy in migrating through semi-permeable membranes and effecting sterilization of the interior(s) of a sterilization load.

During a sterilization phase (e.g., a sterilization pulse), increases in pressure may be faster than decreases in pressure (e.g., the rate of pressure increase during a sterilization pulse may be at least 150 millibar/minute faster than the rate of pressure decrease during the same pulse). This may promote travel of the sterilant (e.g., promote travel of sterilant through one or more layers of packaging). During aeration, the reverse may be employed to promote travel of sterilant from within layers of packaging to exterior of the packaging, and through the exhaust of the sterilization apparatus. For example, the rate of pressure decrease during an aeration pulse may be at least 150 millibar/minute faster than the rate of pressure increase during the same pulse. Increased chamber temperatures may also increase the efficiency of aeration.

The saturation level of the sterilization chamber may also factor into the rate or direction of pressure adjustment. For example, pressure increases near atmospheric pressure should be avoided while the sterilization chamber is near saturation. For example, greater pressure changes may be used at lower sterilant concentrations, while large pressure changes at high sterilant concentrations may cause excess condensation, decreasing the efficiency of the sterilization.

Sterilizing agents may also need to be successfully removed from the load to avoid remaining as residue on or in a medical product. For example, in embodiments utilizing packaging including a semi-permeable membrane, traversal of the semi-permeable membrane may only be possible for sterilizing agents in a particular form, such as a vapor or gas. In some cases, stimulating mechanical movement (e.g., rocking, rotation, agitation, etc.) of some or all of a load may dislodge sterilant molecules adhered to a load, and may promote aeration and removal of a sterilant from the load. Low frequency pressure waves or acoustics generated within the sterilization chamber (for example, via a diaphragm or piston located within the chamber) may dislodge sterilant adhered to the load.

Immediately increasing the pressure following a sterilization pulse can cause unnecessary condensation and result in less efficacious aeration. Prior to aeration, the humidity of the sterilization chamber may be reduced to prevent excessive condensation. For example, the contents of the closed system may be passed through a condenser (e.g., a desiccant wheel) to reduce the humidity of the environment. In addition or alternatively, dry air may be injected, prior to, or during exhaustion, to reduce the overall humidity of the system.

The effects of cold spots or areas of relative warmth and coolness on the distribution and movement of sterilant during sterilization processes may be more pronounced in sterilization loads including relatively greater masses of liquid medicament, and/or relatively greater ratios of medicament mass to total mass of the sterilization load. Accordingly, the parameters of sterilization methods should be tailored to characteristics of the sterilization load.

Systems and methods disclosed herein may advantageously be used in improving the efficacy of sterilization, aeration, and/or drying cycles involving vaporized chemical sterilant. For example, systems and methods of the present disclosure may inject more total vaporized sterilant, and provide more sterilant exposure time to the product load, resulting in effective sterilization and aeration within a shorter total cycle duration, compared to conventional methods and systems. In particular, systems and methods disclosed herein may provide improved sterilization efficiency for liquid heavy sterilization loads.

For example, systems and methods disclosed herein may provide for full (e.g., 100%) sterilization of medical products using VHP, followed by full (e.g., 100%) removal of VHP from sterilized products. Systems and methods disclosed herein may, e.g., increase efficiency, safety, and efficacy of sterilization, and/or decrease sterilization cycle time. While aspects of the present disclosure may be described with respect to the use of VHP in terminal sterilization of PFS, the present disclosure contemplates using the techniques and systems herein for movement of VHP and other chemical sterilants in other contexts as well (e.g., sterilization of other products, cleaning areas, addition/removal of vaporized chemical to any environment, etc.).

The present disclosure also contemplates performance of “moist chemical sterilization,” by which chemical sterilization may be achieved in the presence of water vapor. Comparison of “moist chemical sterilization” to “chemical sterilization” may be analogous, in some cases, to comparison of “moist heat sterilization” to “heat sterilization.” In some instances, moist chemical sterilization may be a more effective and efficient means of achieving sterilization than chemical sterilization technology that currently exists, in the same way that “moist heat sterilization” is considered to be, in some cases, more effective and efficient than only “heat sterilization.”

“Moist chemical sterilization” may take place when environmental conditions of relatively high chemical concentration, high water vapor concentration, and high pressure (e.g., above 400 millibar) act in concert to force the chemical and water vapor to behave as a binary mixture. In order to achieve the desired relatively high chemical concentration, high water vapor concentration, and high pressure, the area to be sterilized may be saturated with a combination of water vapor and sterilizing chemical (e.g., VHP), forcing vapor to condense on surfaces of the load. Most commercially available hydrogen peroxide is available and sold as aqueous liquid mixtures in varying concentrations (e.g., 3%, 15%, 35%, 59%), and thus, vaporizing hydrogen peroxide will generally simultaneously include vaporizing water.

In some embodiments, a method of sterilizing a packaging assembly housing a drug delivery device may comprise:
positioning the packaging assembly inside a sterilization chamber, wherein the drug delivery device housed within the packaging assembly is pre-filled with a VEGF antagonist;
sterilizing the packaging assembly by conducting: a primary sterilant exposure that includes: adjusting a pressure in the sterilization chamber to a first sterilization pressure; injecting at least a first amount of a gaseous sterilant into the sterilization chamber; and injecting a first amount of a distribution gas into the sterilization chamber to raise the pressure in the sterilization chamber to a first transition pressure that is greater than the first sterilization pressure; a secondary sterilant exposure that includes: adjusting the pressure in the sterilization chamber to a second sterilization pressure; injecting at least a second amount of the gaseous sterilant into the sterilization chamber; and injecting a second amount of the distribution gas into the sterilization chamber to raise the pressure in the sterilization chamber to a second transition pressure that is greater than the second sterilization pressure.

In a further embodiment of the method, each of the primary sterilant exposure and the secondary sterilant exposure comprises: directing the gaseous sterilant through a removable cover of the packaging assembly and into a cavity of the packaging assembly that houses the drug delivery device, wherein the drug delivery device includes a body that is silicone-free and stores the VEGF antagonist.

In a further embodiment of the method, the first sterilization pressure and the second sterilization pressure each includes at least about 500 millibars, and the first transition pressure and the second transition pressure each include at least about 900 millibars.

In a further embodiment of the method, the gaseous sterilant includes vaporized hydrogen peroxide; and wherein the first amount of the gaseous sterilant includes between about 125 grams to about 150 grams of the vaporized hydrogen peroxide, and the second amount of the gaseous sterilant includes between about 50 grams to about 75 grams of the vaporized hydrogen peroxide.

In a further embodiment of the method, prior to sterilizing the packaging assembly, the method comprises: conditioning the sterilization chamber by: adjusting the pressure in the sterilization chamber to a conditioning pressure; and adding a conditioning gas into the sterilization chamber to raise the pressure in the sterilization chamber to at least about 150 millibar greater than the conditioning pressure.

In a further embodiment of the method, the conditioning pressure is between about 450 millibars to about 650 millibars.

In a further embodiment of the method, conditioning the sterilization chamber includes one of a plurality of conditioning pulses.

In a further embodiment of the method, the first transition pressure is at least about 400 millibar greater than the first sterilization pressure, and the second transition pressure is at least about 400 millibar greater than the second sterilization pressure.

In a further embodiment of the method, the method further comprises: aerating the sterilization chamber by: adjusting the pressure within the sterilization chamber to a first aeration pressure that is less than or equal to the first sterilization pressure; and adding an aeration gas into the sterilization chamber to raise the pressure in the sterilization chamber to an exhaust pressure that is greater than the first sterilization pressure.

In a further embodiment of the method, the first aeration pressure includes between about 400 millibars to about 500 millibars, and the exhaust pressure includes between about 700 millibars to about 1000 millibars.

In a further embodiment of the method, aerating the sterilization chamber further comprises: adjusting the pressure within the sterilization chamber to a second aeration pressure over an exhaust time, wherein the second aeration pressure is equal to or greater than the first aeration pressure.

In a further embodiment of the method, the second aeration pressure is at least 900 millibar, and the exhaust time is at least about 25 minutes.

In a further embodiment of the method, the method further comprises: sterilizing the packaging assembly by conducting a tertiary sterilant exposure that includes: adjusting the pressure in the sterilization chamber to a third sterilization pressure; injecting at least a third amount of the gaseous sterilant into the sterilization chamber; and injecting a third amount of the distribution gas into the sterilization chamber to raise the pressure in the sterilization chamber to a third transition pressure that is greater than the second sterilization pressure.

In a further embodiment of the method, the third transition pressure is at least about 400 millibar greater than the third sterilization pressure.

In a further embodiment of the method, a total amount of the gaseous sterilant injected into the sterilization chamber during the primary sterilant exposure, the secondary sterilant exposure, and the tertiary sterilant exposure is greater than or equal to about 800 grams.

Evaluation & Amin of Dose Delivery Device with Medicament

In some embodiments, the purity of the devices may be evaluated. For example, the devices may have a purity evaluated by reduced capillary electrophoresis sodium dodecyl sulfate (CE-SDS). The purity may be in the range from approximately 95.08% to 95.66%. The R1 (region 1) purity may be in the range from approximately 0.92% to 1.23%. The low molecular weight (LMW) purity less the region 1 purity may be in the range from approximately 3.42% to 3.71%.

In some embodiments, the devices may have a purity evaluated by non-reduced capillary electrophoresis sodium dodecyl sulfate (CE-SDS). For example, the main peak purity may be in the range from approximately 99.09% to 99.34%. In some embodiments, the devices may have a purity evaluated by size-exclusion ultra-performance liquid chromatography (SE-UPLC). For example, the main peak purity may be in the range from approximately 98.0% to 98.8%, and the aggregate purity may be approximately 1.2% to 2.0%.

In some embodiments, the change variants may be evaluated by imaged capillary isoelectric focusing (iCIEF). For example, the change variants may be evaluated in three regions. Region 1 (acidic) may have a percent change variant in the range from approximately 29.48% to 31.38%. Region 2 (neutral) may have a percent change variant in the range from approximately 43.78% to 45.50%. Region 3 (basic) may have a percent change variant in the range from approximately 23.31% to 26.06%.

In further embodiments, batches of the device may be evaluated for their physical properties, identity, total protein content, container closure integrity, maximum use torque, and dye leak of the blister. For example, the contents of the batches may be found to have liquid essentially free from visible particulates. The batches of devices may have a clarity of less than or equal to III. The batches of devices may have a color of less than or equal to BY5. The pH of the contents of the batches of devices may have a pH of approximately 5.9 and may conform to dot blot tests. The total protein contents, measured in absorbance at 280 nm, may be in the range from approximately 113.0 mg/mL to 113.5 mg/mL. The container closure integrity of the batches of devices may be evaluated such that no leaks may be detected. The maximum use torque may be less than or equal to approximately 13 N·cm. In some embodiments, the batches of blister packs may be found to have no dye detected.

In some embodiments, batches of devices according to the present disclosure may have a potency in the range from approximately 109% to 113%. The isoaspartate content in mol isoaspartate/mol aflibercept of the batches of device may be in the range from approximately 0.06 to 0.08. The particulate matter of the contents of the devices may be evaluated based on particles/mL, including particles at least equal to or greater than 10 μm, at least equal to or greater than 25 μm, and/or at least equal to or greater than 50 μm. The contents of the batches of devices may have a quantity of particles at least equal to or greater than 10 μm in the range from approximately 5 to 8; approximately 2 particles of at least equal to or greater than 25 km; and/or a quantity of particles at least equal to or greater than 50 m in the range from approximately 0 to 1. The volume of the contents of the deliverable volume in the batches of syringes may be in the range from approximately 0.079 mL to 0.081 mL. The maximum injection force of the syringe may be in the range from approximately 14.2 N to 19.2 N. The contents of the device may be considered sterile when the sterility of the container is found to meet USP and/or Ph. Eur. Requirements. The endotoxin content may be in the range from less than approximately 0.05 EU/mL to less than 0.15 EU/mL.

In other embodiments, the purity of the batches of devices may be evaluated. For example, the devices may have a purity evaluated by reduced capillary electrophoresis sodium dodecyl sulfate (CE-SDS). The purity may be in the range from approximately 95.08% to 95.2%. The region 1 purity may be in the range from approximately 1.1% to 1.2%. The low molecular weight purity less the region 1 purity may be in the range from approximately 3.7% to 3.71%. In some embodiments, the batches of the devices may have a purity evaluated by non-reduced capillary electrophoresis sodium dodecyl sulfate (CE-SDS). For example, the main peak purity may be in the range from approximately 98.9% to 99.3%.

In some embodiments, the batches of the devices may have a purity evaluated by size-exclusion ultra-performance liquid chromatography (SE-UPLC). For example, the main peak purity may be in the range from approximately 97.2% to 98.1%. The aggregate purity may be approximately 1.9% to 2.8%.

In some embodiments, the change variants of the batches of devices may be evaluated by imaged capillary isoelectric focusing (iCIEF). For example, the change variants may be evaluated in three regions. Region 1 may have a percent change variant in the range from approximately 29.9% to 31.4%. Region 2 may have a percent change variant in the range from approximately 44.7% to 45.3%. Region 3 may have a percent change variant in the range from approximately 23.3% to 25.4%.

Devices according to the present disclosure may be evaluated according to the International Council of Harmonisation (ICH) Q5C recommended long-term and accelerated storage conditions maintained by qualified controlled temperature chambers to establish a stability profile. The shelf life of the device may be determined from the date of manufacture of the syringe and/or the contents of the syringe. In some embodiments, the shelf life of the device may be approximately 24 months when stored at approximately 2° C. to 8° C. In some embodiments, devices according to the present disclosure may be evaluated for approximately 36 months at 5° C. to evaluate the long-term stability and approximately 6 months at 25° C. to evaluate the accelerated stability. The long-term stability may be evaluated to determine the extent of changes to quality during long-term storage at the recommended storage conditions and to establish shelf-life. The accelerated stability may be evaluated to support manufacturing and handling at ambient temperature for limited time periods, to examine the effects of room temperature storage and handling on product quality, and to help identify likely degradation pathways. In some embodiments, the devices may experience no appreciable change in any of the monitored attributes when evaluated for their long-term or accelerated stability. In some embodiments, there may be no meaningful change observed for one or more of appearance (physical form and/or condition), color, clarity, pH, total protein, charge variants, polysorbate 20 content, and particulate matter observed after 6 months at the accelerated conditions.

Under accelerated conditions, a decrease in potency by bioassay may be observed after 6 months. A decrease in purity and an increase in LMW species may be observed by reduced capillary electrophoresis sodium dodecyl sulfate. A decrease in purity may be observed by non-reduced Capillary electrophoresis sodium dodecyl sulfate after 6 months. A decrease in purity and an increase in aggregate species may be observed by size exclusion ultra-performance liquid chromatography after 6 months. An increase in isoaspartate content may be observed after 6 months. A decrease in max injection force may be observed after 6 months. Accordingly, it should be appreciated that devices of the present disclosure may be stable at the long-term storage conditions due to the stability data showing little to no change in quality attributes.

Devices of the present disclosure may be evaluated after various time periods. The following examples are intended to illustrate the present disclosure without being limiting in nature. It is understood that the present disclosure encompasses additional aspects and embodiments consistent with the foregoing description and following examples.

### Example 11

In one example, a plunger rod and flange piece were evaluated for aging under approximately 55° C. and 50% relative humidity. The aging test method may include one or more of the following aspects: evaluating the override torque, the force required to over-torque the plunger rod in the ready to dose state; evaluating the syringe assembly force, the force required to assemble the syringe into the finger flange; and the plunger rod pullout force, the force required to move the plunger rod from the finger flange in the new state. As an illustrative example, an exemplary summary of these results are shown in Table 10.

TABLE 10

| | Summary | Timepoint (months) | | | |
|---|---|---|---|---|---|
| Test | Statistics | t = 0 | t = 12 | t = 36 | t = 60 |
| Override | Mean | 28.537 | NA | NA | 28.970 |
| Torque (N) | Standard | 1.943 | | | 2.694 |
| | deviation | | | | |
| | Minimum | 22.03 | | | 22.52 |
| | Maximum | 31.05 | | | 31.91 |
| Syringe | Mean | 15.528 | 15.628 | 15.241 | 16.251 |
| Assembly | Standard | 2.048 | 2.339 | 1.544 | 1.562 |
| Force (N) | deviation | | | | |
| | Minimum | 12.799 | 11.496 | 12.101 | 13.811 |
| | Maximum | 19.077 | 20.439 | 18.159 | 19.079 |
| Plunger Rod | Mean | 46.212 | NA | NA | 45.144 |
| Pullout | Standard | 6.118 | | | 1.107 |
| Force (N) | deviation | | | | |
| | Minimum | 36.252 | | | 42.929 |
| | Maximum | 57.226 | | | 48.081 |

### Example 12

In one example, delivery device 2800 and packaging 3000 may be evaluated for aging under ASTM F1980-21 (Standard Guide for Accelerated Aging of Sterile Barrier Systems for Medical Devices) at 50% relative humidity. To investigate the effects of long-term storage, the packaged devices were stored at 50% relative humidity. In accordance with ASTM F1980-21, testing was first performed after 63 days of accelerated aging conditions to simulate a minimum of 3 years (36 months, 1095 days,) of real-time aging. All tested and inspected samples met the acceptance criteria after accelerated aging. As an illustrative example, an exemplary summary of these results are shown in Table 11.

TABLE 11

| Requirement | Summary Statistics | Acceptance Criteria | Result |
|---|---|---|---|
| The packed and labeled product | N/A | A = 0, R = 1 | Pass |
| must be sterile^{a} | | | |
| The secondary packaging carton | N/A | A = 0, R = 1 | Pass |
| shall contain and demonstrate | | | |
| tamper evidence^{b} | | | |
| The blister lid shall have a peel- | K ≥ 2.555 | K = 6.591 | Pass |
| off force of 1.6 ≤ x ≤ 23 N post- | | | |
| transportation and throughout | | | |
| shelf life^{a} | | | |
| The PFS label linear bar code | N/A | A = 0, R = 1 | Pass |
| shall be scannable and contain | | | |
| the appropriate NDC^{b} | | | |
| The blister lid linear bar code | N/A | A = 0, R = 1 | Pass |
| shall be scannable and contain | | | |
| the appropriate NDC^{b} | | | |
| The carton lid linear bar code | N/A | A = 0, R = 1 | Pass |
| shall be scannable and contain | | | |
| the appropriate NDC^{b} | | | |
| The labels shall remain attached | N/A | A = 0, R = 1 | Pass |
| to the product post- | | | |
| transportation and throughout | | | |
| shelf life^{b} | | | |
| The device label and secondary | N/A | A = 0, R = 1 | Pass |
| packaging content shall be | | | |
| legible throughout shelf life and | | | |
| transportation^{c} | | | |
| The blister package shall | N/A | A = 0, R = 1 | Pass |
| accommodate the assembled | | | |
| device^{c} | | | |

^{a}n = 30;
^{b}n = 60;
^{c}n = 120

### Example 13

In another example, the device was functionally tested after aging. A total of 30 devices were tested per test point. For attribute testing, sample sizes used during design verification ensured that a probability content based on risk level (95% for minor risk, 97.5% for medium risk, or 99% for high risk) is compliant to the acceptance criteria considering a confidence level of 95%. For variable testing, the design verification test results are evaluated through statistical tolerance interval calculation considering a confidence level of 95% and a probability content based on the respective risk level (95% for minor risk, 97.5% for medium risk, and 99% for high risks). As an illustrative example, exemplary results of the functional testing are shown in Table 12.

TABLE 12

| | Acceptance | Test | Probability | Summary | Acceptance | |
|---|---|---|---|---|---|---|
| Requirement | Criteria | Group | Content | Statistics | Criteria | Results |
| Deliverable | 0.070 mL ≤ | t = 0 | 97.5% | Mean: 0.086 mL | K ≥ 2.291 | K = 14.835 |
| Volume | X < 0.10 mL | | | Std dev: 0.001 mL | | |
| | | | | Range: 0.084-0.089 mL | | |
| | | Aging | | Mean: 0.081 mL | | K = 3.637 |
| | | (6 mo.) | | Std dev: 0.003 mL | | |
| | | | | Range: 0.071 mL-0.085 mL | | |
| | | Aging | | Mean: 0.084 nL | | K = 5.364 |
| | | (12 mo.) | | Std dev: 0.003 mL | | |
| | | | | Range: 0.075-0.088 mL | | |
| | | Aging | | Mean: 0.084 mL | | K = 7.602 |
| | | (24 mo.) | | Std dev: 0.002 mL | | |
| | | | | Range: 0.079-0.086 mL | | |
| | | Aging | | Mean: 0.085 mL | K ≥ 3.654 | K = 7.095 |
| | | (36 mo.) | | Std dev: 0.002 mL | | |
| | | | | Range: 0.078-0.087 mL | | |
| Maximum | ≤28.2 N | t = 0 | 97.5% | Mean: 13.1 N | K ≥ 2.608 | K = 8.952 |
| Injection | | | | Std dev: 1.69 N | | |
| Force | | | | Range: 10.04-15.70 N | | |
| | | Aging | | Mean: 14.5 N | | K = 11.902 |
| | | (6 mo.) | | Std dev: 1.2 N | | |
| | | | | Range: 9.9-16.5 N | | |
| | | Aging | | Mean: 13.2 N | | K = 13.091 |
| | | (12 mo.) | | Std dev: 1.1 N | | |
| | | | | Range: 10.6-16.2 N | | |
| | | Aging | | Mean: 11.6 N | | K = 17.086 |
| | | (24 mo.) | | Std dev: 0.97 N | | |
| | | | | Range: 9.01-14.08 N | | |
| | | Aging | | Mean: 15.3 N | | K = 10.496 |
| | | (36 mo.) | | Std dev: 1.23 N | | |
| | | | | Range: 11.7-17.8 N | | |
| The Snap-Off | The snap-off | All ages | 95% | N/A | A = 0, R = 1 | Pass |
| Tip Cap Shall | tip cap shall | | | | | |
| Be Removable | be removable | | | | | |
| at Time of Use | at time of use. | | | | | |
| Plunger Rod | ≤13 N · cm | t = 0 | 95% | Mean: 1.6 N · cm | K ≥ 4.022 | K = 28.550 |
| Twisting | | | | Std dev: 0.40 N · cm | | |
| Torque | | | | Range: 1.2-3.6 N · cm | | |
| | | Aging | | Mean: 2.2 N · cm | K ≥ 2.220 | K = 97.492 |
| | | (6 mo.) | | Std dev: 0.11 N · cm | | |
| | | | | Range : 1.9-2.4 N · cm | | |
| | | Aging | | Mean: 1.9 N · cm | | K = 73.969 |
| | | (12 mo.) | | Std dev: 0.15 N · cm | | |
| | | | | Range: 1.5-2.2 N · cm | | |
| | | Aging | | Mean: 2.1 N · cm | | K = 60.906 |
| | | (24 mo.) | | Std dev: 0.18 N · cm | | |
| | | | | Range: 1.7-2.5 N · cm | | |
| | | Aging | | Mean: 1.7 N · cm | | K = 52.147 |
| | | (36 mo.) | | Std dev: 0.22 N · cm | | |
| | | | | Range: 1.2-2.2 N · cm | | |
| Plunger Rod | >13 N · cm | t = 0 | 95% | Mean: 27.5 N · cm | K ≥ 2.220 | K = 7.578 |
| Twisting | | | | Std dev: 1.9 N · cm | | |
| Override | | | | Range: 23.0-31.4 N · cm | | |
| Torque | | Aging | | Mean: 26.5 N · cm | | K = 10.365 |
| | | (6 mo.) | | Std dev: 1.3 N · cm | | |
| | | | | Range: 24.8-30.2 N · cm | | |
| | | Aging | | Mean: 27.4 N · cm | | K = 7.872 |
| | | (12 mo.) | | Std dev: 1.8 N · cm | | |
| | | | | Range: 24.3-31.2 N · cm | | |
| | | Aging | | Mean: 26.0 N · cm | | K = 5.571 |
| | | (24 mo.) | | Std dev: 2.3 N · cm | | |
| | | | | Range: 20.5-29.5 N · cm | | |
| | | Aging | | Mean: 26.5 N · cm | | K = 7.670 |
| | | (36 mo.) | | Std dev: 1.7 N · cm | | |
| | | | | Range: 22.2-29.2 N · cm | | |

### Example 14

In some examples, pre-filled syringe (PFS) drug products (DP) may be evaluated in bulk and/or individually for a variety of characteristics. As an illustrative example, exemplary results of functional testing are shown in Table 13.

TABLE 13

| | | PFS Acceptance Criteria | | |
|---|---|---|---|---|
| | | Release | Release | |
| | | Testing of | Testing of | End of Shelf |
| Test | Method | Bulk PFS | PFS | Life of PFS |
| Physical | Visual | Liquid | Results reported from | Same as |
| Form/ | inspection | essentially free | bulk PFS | release |
| Condition | USP <790> | from visible | | |
| | Ph. Eur. 2.9.20 | particulates | | |
| | JP <6.06> | | | |
| Clarity | Comparison to | Not more | Results reported from | Same as |
| | reference | turbid than | Bulk PFS | release |
| | suspension | reference | | |
| | USP <855> | suspension IV | | |
| | Ph. Eur. 2.2.1 | | | |
| | JP <2.61> | | | |
| Color | Comparison to | Not more | Results reported from | Same as |
| | reference | colored than | Bulk PFS | release |
| | solutions | reference | | |
| | USP <1061> | solution BY4 | | |
| | Ph. Eur. 2.2.2 | | | |
| | JP <2.65> | | | |
| pH | Potentiometry | 5.6-6.1 | Results reported from | Same as |
| | USP <791> | | Bulk PFS | release |
| | Ph. Eur. 2.2.3 | | | |
| | JP <2.54> | | | |
| Identity by | Dot blot | Conforms to | Conforms to standard | Not tested |
| Dot Blot | | standard | | |
| Total | UV | 103.0-126.0 | Results reported from | Same as |
| Protein | spectrophotometry | mg/mL | Bulk PFS | release |
| Content | | | | |
| (A₂₈₀) | | | | |
| Potency by | Cell based | 70-130% | Results reported from | Same as |
| Bioassay | bioassay | of reference | Bulk PFS | release |
| | | standard | | |
| Purity by | Capillary | a. ≥93.0% | Results reported from | a. ≥91.5% |
| Reduced | electrophoresis - | b. ≤2.2% | Bulk PFS | b. ≤2.5% |
| CE-SDS | sodium dodecyl | c. ≤5.0% | | c. ≤6.0% |
| a. % Purity | sulfate | | | |
| b. % R1 | | | | |
| c. % LMW | | | | |
| Less R1 | | | | |
| Purity by | | a. ≥97.5% | Results reported from | a. ≥96.5% |
| Non- | | | Bulk PFS | |
| reduced CE- | | | | |
| SDS | | | | |
| a. % Main | | | | |
| Peak Purity | | | | |
| Purity by | Liquid | a. ≥97.0% | Results reported from | a. ≥94.0% |
| SE-UPLC | chromatography | b. ≤3.0% | Bulk PFS | b. ≤5.0% |
| a. % Purity | | | | |
| b. % | | | | |
| Aggregate | | | | |
| Charge | Isoelectric | The test article | Results reported from | Same as |
| Variant | focusing | profile should | Bulk PFS | release |
| Analysis by | | be qualitatively | | |
| iCIEF | | similar to the | | |
| a. % | | Reference | | |
| Region 1 | | Standard | | |
| b. % | | electropherogram | | |
| Region 2 | | in terms of | | |
| c. % | | intensity, | | |
| Region 3 | | number, and | | |
| | | pattern of | | |
| | | peaks. | | |
| | | a. 21.5-38.4% | | |
| | | b. 38.5-50.4% | | |
| | | c. 13.5-36.4% | | |
| Polysorbate | Mixed mode | Not tested | Not tested | 0.022-0.038% |
| 20 | chromatography | | | |
| | with evaporative | | | |
| | light scattering | | | |
| | detection | | | |
| | (ELSD) | | | |
| Isoaspartate | Enzyme-linked | ≤0.12 mol | Results reported from | ≤0.22 mol |
| | detection of | isoaspartate/mol | Bulk PFS | isoaspartate/ |
| | isoaspartate | aflibercept | | mol aflibercept |
| | with reverse | | | |
| | phase | | | |
| | HPLC/UV | | | |
| Endotoxin | KTA | ≤0.2 | Results reported from | Not tested |
| Content | USP <85> | EU/mL | Bulk PFS | |
| | Ph. Eur. 2.6.14 | | | |
| | JP <4.01> | | | |
| Sterility | Membrane | Meets USP and | Results reported from | Not tested |
| (Filled | filtration | Ph. Eur. | Bulk PFS | |
| Container) | USP <71> | requirements | | |
| | Ph. Eur. 2.6.1 | | | |
| | JP <4.06> | | | |
| Particulate | Microscopic | a. ≤50 | Results reported from | Same as |
| Matter | USP <787> | particles/mL | Bulk PFS | release |
| (Microscopic) | USP <788> | b. ≤5 | | |
| a. Particles | USP <789> | particles/mL | | |
| ≥10 μm | Ph. Eur. 2.9.19 | c. ≤2 | | |
| b. Particles | JP <6.07> | particles/mL | | |
| ≥25 μm | | | | |
| c. Particles | | | | |
| ≥50 μm | | | | |
| Volume in | Volume in | 0.07 mL | Not tested | Not tested |
| Container | container | minimum | | |
| | USP <697> | expellable | | |
| | Ph. Eur. 2.9.17 | volume | | |
| | JP <6.05> | | | |
| Container | Mass extraction | Not tested | No leak detected | Same as |
| Closure | USP <1207.2> | | | release |
| Integrity | | | | |
| Maximum | Determination | Not tested | ≤28.2 N | Same as |
| Injection | of maximum | | | release |
| Force | injection force | | | |
| | ISO 11040-4 | | | |
| | ISO 11040-8 | | | |
| Deliverable | Measurement of | N/A | 0.070 mL ≤ | Same as |
| Volume | expelled liquid | | X < 0.10 mL | release |
| | USP <697> | | | |
| | ISO 11040-8 | | | |
| Maximum | Force | N/A | ≤13 | Same as |
| Use Torque | measurement | | N · cm | release |
| Dye Leak | Package | N/A | No dye detected | Same as |
| (Blister) | integrity | | | release |
| | USP <1207.2> | | | |

### Example 15

In other embodiments, pre-filled syringe (PFS) drug products (DP) may be evaluated in bulk and individually for a variety of characteristics when stored at 5° C. As an illustrative example, exemplary results of the functional testing are shown in Table 14. The drug products may be composed of 114.3 mg/mL aflibercept (as an active ingredient), in an aqueous buffered solution, pH 5.8, containing 50 mM arginine monohydrochloride (as a stabilizing agent), 10 mM histidine (as a buffering agent), 5% (w/v) sucrose (as a stabilizing agent), and 0.03% (w/v) polysorbate 20 (as a stabilizing agent), and may be stored in a prefilled syringe. Of the 10 mM histidine, the drug products may include 3.65 mM L-histidine and 6.35 mM L-histidine monohydrochloride monohydrate (as buffering agents). The syringe may be a 0.5 mL Type 1 glass syringe barrel protected by a cap closure; have an elastomeric plunger stopper (4023/50 formulation) with FluroTec® barrier film; be assembled with a plunger rod and finger flange; packaged in a blister tray with a Tyvek® lid; and be sterilized with VHP. The syringe may be stored at 5° C. and stored horizontally.

TABLE 14

| | End-of-Shelf-Life | | | | |
|---|---|---|---|---|---|
| | Acceptance | Length of Storage (months) | | | |
| Assay | Criteria | t = 0 | Initial/11 | 12 | 18 |
| Physical | Liquid essentially | LEFFVP | LEFFVP | LEFFVP | LEFFVP |
| Form/ | free from visible | | | | |
| Condition | particulates | | | | |
| (Appearance) | | | | | |
| Clarity | Not more turbid | Not > | Not > | Not > | Not > |
| (Turbidity) | than Reference | III | III | III | III |
| | Suspension IV | | | | |
| Color | Not more | Not > | Not > | Not > | Not > |
| | intensely colored | BY5 | BY5 | BY5 | BY5 |
| | than Reference | | | | |
| | Solution BY4 | | | | |
| pH | 5.6-6.1 | 5.9 | 5.9 | 5.9 | 5.9 |
| Total Protein | 103.0-126.0 | 113.0 | 114.4 | 114.0 | 113.4 |
| Content (A₂₈₀) | mg/mL | | | | |
| Potency by | 70%-130% of | 113 | 95 | 85 | 75 |
| Bioassay | reference standard | | | | |
| Purity by | a. ≥91.5% purity | 95.1 | 95.1 | 94.7 | 94.5 |
| Reduced CE- | b. ≤2.5% | 1.2 | 1.2 | 1.2 | 1.4 |
| SDS | c. ≤6.0% | 3.7 | 3.7 | 4.0 | 4.1 |
| a. % Purity | | | | | |
| b. % R1 | | | | | |
| c. % LMW | | | | | |
| less R1 | | | | | |
| Purity by | a. % Main peak | 99.3 | 98.9 | 98.7 | 98.9 |
| Non-Reduced | purity ≥96.5% | | | | |
| CE-SDS | total area | | | | |
| a. % Main | | | | | |
| peak purity | | | | | |
| Purity by SE- | a. aflibercept main | 98.0 | 97.2 | 97.1 | 96.6 |
| UPLC | peak ≥94.0% | | | | |
| a. % Main | total area | | | | |
| Peak | b. ≤5.0% | 2.0 | 2.8 | 2.9 | 3.3 |
| b. % | aggregate | | | | |
| Aggregate | | | | | |
| Charged | The test article | Conforms | Conforms | Conforms | Conforms |
| Variant | profile should be | | | | |
| Analysis by | qualitatively | | | | |
| iCIEF | similar to the | | | | |
| a. % Region 1 | Reference | | | | |
| b. % Region 2 | Standard | | | | |
| c. % Region 3 | electropherogram | | | | |
| | in terms of | | | | |
| | intensity, number, | | | | |
| | and pattern of | | | | |
| | peaks. | | | | |
| | a. 21.5-38.4% | 29 | 30 | 29 | 30 |
| | b. 38.5-50.4% | 45 | 45 | 45 | 45 |
| | c. 13.5-36.4% | 25 | 25 | 26 | 25 |
| Isoaspartate | ≤0.22 mol | 0.08 | 0.11 | 0.12 | 0.13 |
| | isoaspartate/mol | | | | |
| | aflibercept | | | | |
| Polysorbate | 0.022%-0.038% | NR | 0.027 | 0.030 | 0.032 |
| 20 | (w/v) | | | | |
| Particulate | ≤50 particles/mL | 5 | 8 | 3 | 2 |
| Matter | (≥10 μm) | | | | |
| (microscopic) | ≤5 particles/mL | 2 | 1 | 0 | 1 |
| | (≥25 μm) | | | | |
| | ≤2 particles/mL | 0 | 1 | 0 | 0 |
| | (≥50 μm) | | | | |
| Container | No leak detected | NR | NLD | NLD | NR |
| Closure | | | | | |
| Integrity | | | | | |
| (CCI) | | | | | |
| Max Injection | ≤28.2 Newtons | NR | 12.7 | 15.0 | 15.0 |
| Force | (N) | | | | |
| Deliverable | 0.07 mL ≤ | NR | 0.086 | 0.087 | 0.086 |
| Volume | X < 0.10 mL | | | | |
| Maximum | ≤13 N · cm | NR | ≤13 N · cm | ≤13 N · cm | ≤13 N · cm |
| Use Torque | | | | | |
| Dye Leak | No dye detected | NR | NDD | NDD | NR |
| (Blister) | | | | | |

### Example 16

In further embodiments, pre-filled syringe (PFS) drug products (DP) were evaluated in bulk and individually for a variety of characteristics when stored at 25° C. As an illustrative example, exemplary results of the functional testing are shown in Table 15. The drug products may be composed of 114.3 mg/mL aflibercept, in an aqueous buffered solution, pH 5.8, containing 50 mM arginine monohydrochloride, 10 mM histidine, 5% (w/v) sucrose, and 0.03% (w/v) polysorbate 20 and may be stored in a prefilled syringe. The syringe may be a 0.5 mL Type 1 glass syringe barrel protected by a cap closure; have an elastomeric plunger stopper (4023/50 formulation) with FluroTec® barrier film; be assembled with a plunger rod and finger flange; packaged in a blister tray with a Tyvek® lid; and be sterilized with VHP. The syringe may be stored at 25° C. and stored horizontally.

TABLE 15

| | End-of-Shelf-Life | | | | |
|---|---|---|---|---|---|
| | Acceptance | Length of Storage (months) | | | |
| Assay | Criteria^{a} | t = 0^{b} | 1 | 3 | 6 |
| Physical Form/ | Liquid essentially | LEFFVP | LEFFVP | LEFFVP | LEFFVP |
| Condition | free from visible | | | | |
| (Appearance) | particulates | | | | |
| Clarity | Not more turbid | Not > | Not > | Not > | Not > |
| (Turbidity) | than Reference | III | III | III | III |
| | Suspension IV | | | | |
| Color | Not more intensely | Not > | Not > | Not > | Not > |
| | colored than | BY5 | BY5 | BY5 | BY5 |
| | Reference Solution | | | | |
| | BY4 | | | | |
| pH | 5.6-6.1 | 5.9 | 5.9 | 5.9 | 5.9 |
| Total Protein | 103.0-126.0 | 113.0 | 114.4 | 113.6 | 115.2 |
| Content (A₂₈₀) | mg/mL | | | | |
| Potency by | 70%-130% of | 113 | 86 | 91 | 79 |
| Bioassay | reference standard | | | | |
| Purity by | a. ≥91.5% purity | 95.1 | 94.1 | 92.9 | 91.1 |
| Reduced CE- | b. ≤2.5% | 1.2 | 1.6 | 2.1 | 2.6 |
| SDS | c. ≤6.0% | 3.7 | 4.3 | 4.9 | 6.3 |
| a. % Purity | | | | | |
| b. % R1 | | | | | |
| c. % LMW less | | | | | |
| R1 | | | | | |
| Purity by Non- | a. % Main peak | 99.3 | 98.5 | 98.2 | 96.9 |
| Reduced CE- | purity ≥96.5% total | | | | |
| SDS | area | | | | |
| a. % Main peak | | | | | |
| purity | | | | | |
| Purity by SE- | a. aflibercept main | 98.0 | 96.3 | 94.5 | 91.9 |
| UPLC | peak ≥94.0% total | | | | |
| a. % Main Peak | area | | | | |
| b. % Aggregate | b. ≤5.0% aggregate | 2.0 | 3.4 | 5.0 | 7.4 |
| Charged | The test article | Conforms | Conforms | Conforms | Conforms |
| Variant | profile should be | | | | |
| Analysis by | qualitatively similar | | | | |
| iCIEF | to the Reference | | | | |
| a. % Region 1 | Standard | | | | |
| b. % Region 2 | electropherogram in | | | | |
| c. % Region 3 | terms of intensity, | | | | |
| | number, and pattern | | | | |
| | of peaks. | | | | |
| | a. 21.5-38.4% | 29 | 29 | 30 | 30 |
| | b. 38.5-50.4% | 45 | 45 | 45 | 46 |
| | c. 13.5-36.4% | 25 | 26 | 0.26 | 0.41 |
| Isoaspartate | ≤0.22 mol | 0.08 | 0.16 | 0.26 | 0.41 |
| | isoaspartate/mol | | | | |
| | aflibercept | | | | |
| Polysorbate 20 | 0.022%-0.038% | n/a | 0.030 | 0.028 | 0.032 |
| | (w/v) | | | | |
| Particulate | ≤50 particles/mL | 5 | 5 | 1 | 1 |
| Matter | (≥10 μm) | | | | |
| (microscopic) | ≤5 particles/mL | 2 | 1 | 0 | 1 |
| | (≥25 μm) | | | | |
| | ≤2 particles/mL | 0 | 0 | 0 | 0 |
| | (≥50 μm) | | | | |
| Maximum | ≤28.2 Newtons (N) | n/a | n/a | n/a | 15.0 |
| Injection Force | | | | | |
| Deliverable | 0.07 mL ≤ | n/a | n/a | n/a | 0.086 |
| Volume | X < 0.10 mL | | | | |
| Maximum Use | ≤13 N · cm | n/a | n/a | n/a | ≤13 N · cm |
| Torque | | | | | |

### Example 17

In some embodiments, devices according to the present disclosure may be evaluated for a variety of quality attributes. As an illustrative example as shown below in Table 16, one or more exemplary test methods may be used for evaluating the various quality attributes of the device, each with a respective rationale.

TABLE 16

| Quality Attribute | Test Method | Rationale |
|---|---|---|
| Physical | Visual inspection | Assessed to describe the physical |
| Form/Condition | USP <790> (United States | state of the solution and ensure |
| | Pharmacopeia) | process consistency. |
| | Ph. Eur. 2.9.20 (European | |
| | Pharmacopoeia) | |
| | JP <6.06> (Japanese | |
| | Pharmacopoeia) | |
| Clarity | Comparison to reference | Assessed to describe the physical |
| | suspension | state of the solution and ensure |
| | USP <855> | process consistency. |
| | Ph. Eur. 2.2.1 | |
| | JP <2.61> | |
| Color | Comparison to reference | Assessed to describe the color of |
| | solutions | aflibercept solution and ensure |
| | USP <1061> | process consistency. |
| | Ph. Eur. 2.2.2 | |
| | JP<2.65> | |
| pH | Potentiometry | The proper pH range of the solution |
| | USP <791> | must be maintained to ensure protein |
| | Ph. Eur. 2.2.3 | stability. |
| | JP <2.54> | |
| Identity | Dot blot | Identifies the presence or absence of |
| | | aflibercept. |
| Total Protein Content | UV (ultraviolet) | Testing ensures protein concentration |
| | spectrophotometry | is suitable for use. |
| | | Formulation development robustness |
| | | studies demonstrated that the |
| | | aflibercept formulation is robust |
| | | against a ± 10% variation in protein |
| | | concentration. |
| Potency | Cell-based bioassay | Bioassay quantifies aflibercept |
| | | activity and ensures product efficacy. |
| Purity | Reduced CE-SDS | Assures purity of the protein. |
| | (capillary electrophoresis- | |
| | sodium dodecyl sulfate) | |
| | Non-reduced | |
| | CE-SDS | |
| | SE-UPLC (size exclusion | |
| | ultra-performance liquid | |
| | chromatography) | |
| LMW (low | Reduced CE-SDS | Assesses levels of LMW species (R1 |
| molecular weight) | | and LMW less R1) under reducing |
| | | conditions. R1 is characterized as |
| | | aflibercept half molecule internally |
| | | clipped within the VEGFR-1 domain. |
| | | LMW less R1 are the remaining |
| | | aflibercept LMW species detected by |
| | | reduced CE-SDS. |
| Aggregate | SE-UPLC | Assesses levels of aggregates |
| | | (characterized as dimeric forms of |
| | | aflibercept in extended |
| | | characterization studies) and some |
| | | higher-order aggregates. |
| Charge Variant | iCIEF (imaged capillary | iCIEF measures the intrinsic charge |
| Analysis | isoelectric focusing) | of the protein to evaluate charge |
| | | heterogeneity, which is related to the |
| | | amino acid composition, |
| | | oligosaccharide groups/sialic acid |
| | | levels, and other PTMs. |
| | | The isoelectric point and |
| | | consequently the charge profile of |
| | | aflibercept is predominantly |
| | | influenced by the heterogeneity of |
| | | sialic acid on N-linked glycans. |
| | | Characterization studies indicate that |
| | | the extent of sialic acid occupancy |
| | | does not affect the potency or purity |
| | | of aflibercept but can impact the |
| | | overall exposure to free drug. |
| | | Based on the potential for sialic acid |
| | | levels to impact pharmacokinetics |
| | | and the influence sialic acid levels |
| | | have on the isoelectric point of |
| | | aflibercept, iCIEF is used to monitor |
| | | the aflibercept charge profile. |
| Polysorbate 20 | Mixed mode | PS20 is a surfactant and interfacial |
| | chromatography with | stabilizing agent which stabilizes the |
| | ELSD (evaporative light | protein with regard to agitation stress. |
| | scattering detection) | End-of-shelf-life testing of DP for |
| | | polysorbate 20 confirms that the level |
| | | of PS20 remains appropriate to |
| | | ensure protein stability. |
| Isoaspartate | Reversed-phase HPLC | This assay provides a measure of the |
| | | amount of isoaspartate in aflibercept, |
| | | which itself is a measure of the |
| | | amount of protein deamidation and |
| | | isomerization. |
| Endotoxin Content | Kinetic turbidimetric assay | To control maximum allowable |
| | USP <85> | endotoxin to below guidelines for |
| | Ph. Eur. 2.6.14 | parenteral products. |
| | JP<4.01> | |
| Sterility | Membrane filtration | Ensures sterility of drug product. |
| | USP <71> | |
| | Ph. Eur. 2.6.1 | |
| | JP <4.06> | |
| Particulate Matter | Microscopic | Provides a measurement of the |
| | USP<787> | particulate matter content. |
| | USP<788> | |
| | USP<789> | |
| | Ph. Eur. 2.9.19 | |
| Volume in Container | USP <697> | Ensures that the volume in container |
| | Ph. Eur. 2.9.17 | is sufficient to provide a dose of |
| | JP <6.05> | aflibercept. |
| Container Closure | Mass extraction | Ensure integrity of container is |
| Integrity | USP <1207.2> | maintained during shelf-life. |
| Maximum Injection | Determination of maximum | Provides a measurement of the |
| Force | injection force | maximum injection force required to |
| | ISO-11040-4 | push a plunger stopper to expel the |
| | ISO 11040-8 | liquid out of the syringe. |
| Deliverable Volume | Measurement of expelled | Ensures the correct dosage of the |
| | liquid | drug is administered to the patient. |
| | USP <697> | |
| | ISO 11040-8 | |
| Maximum Use | Force measurement | Ensure that the maximum force |
| Torque | | required for a 5th percentile female to |
| | | remove a twist off cap with a dry |
| | | hand does not exceed the acceptable |
| | | limit. |
| Dye Leak (Blister) | Package integrity | Ensure integrity of the blister package |
| | USP <1207.2> | seal is maintained during shelf-life. |

In some embodiments, each of a bulk prefilled syringe drug product and a prefilled syringe drug product may be configured to meet a “liquid essentially free from visible particulates” specification according to the United States Pharmacopeia (USP). Additionally, during 100% visual inspection, each syringe may be inspected for the presence of observable foreign and particulate matter in its contents, such that syringes whose contents show evidence of visible particulates may be rejected. The devices may be evaluated based on the bulk prefilled syringe drug product packaging and the prefilled syringe drug product packaging. As an example, Table 17 below shows the results of exemplary quality attribute tests of the different drug product forms.

TABLE 17

| | | Product Form | |
|---|---|---|---|
| | | Bulk Prefilled Syringe | Prefilled Syringe Drug |
| Quality Attribute | | Drug Product | Product |
| Physical Form/Condition | | Liquid essentially free from | Results reported from bulk |
| | | visible particulates | PFS |
| Clarity | | Not more turbid than a | Results reported from bulk |
| | | reference suspension IV, | PFS |
| | | which is one standard | |
| | | greater than III | |
| Color | | Not more colored than | Results reported from bulk |
| | | reference solution BY4, | PFS |
| | | which is one standard | |
| | | greater than BY5 | |
| pH | | 5.6-6.1 | Results reported from bulk |
| | | | PFS |
| Identity by Dot Blot | | Conforms to standard | Conforms to standard |
| Total Protein Content | | 103.0 mg/mL-126.0 | Results reported from bulk |
| | | mg/mL | PFS |
| | | (±10% of the targeted total | |
| | | protein concentration of | |
| | | 114.3 mg/mL) | |
| Potency by Bioassay | | 70%-130% | Results reported from bulk |
| | | | PFS |
| Reduced CE- | % Purity | ≥91.5-93.0 | Results reported from bulk |
| SDS (capillary | | | PFS |
| electrophoresis | % R1 | ≤2.2-2.5 | Results reported from bulk |
| sodium dodecyl | | | PFS |
| sulfate) | % LMW | ≤5.0-6.0 | Results reported from bulk |
| | less R1 | | PFS |
| Non-Reduced | % Purity | ≥96.5-97.5 | Results reported from bulk |
| CE- SDS | | | PFS |
| SE-UPLC | % Purity | ≥94.0-97.0 | Results reported from bulk |
| | | | PFS |
| | % | ≤3.0-5.0 | Results reported from bulk |
| | Aggregate | | PFS |
| iCIEF | % Region 1 | 21.5-38.4 | Results reported from bulk |
| | | | PFS |
| | % Region 2 | 38.5-50.4 | Results reported from bulk |
| | | | PFS |
| | % Region 3 | 13.5-36.4 | Results reported from bulk |
| | | | PFS |

| | | |
|---|---|---|
| Polysorbate 20 Content (% w/v) | 0.022-0.038 | Results reported from bulk |
| | | PFS |
| Isoaspartate | ≤0.12-0.22 | Results reported from bulk |
| | | PFS |
| Endotoxin (EU/mL) | ≤0.2 | Results reported from bulk |
| | | PFS |
| Sterility | Meets USP and Ph. Eur. | Results reported from bulk |
| | requirements | PFS |
| Particles ≥10 μm (particles/mL) | ≤50 | Results reported from bulk |
| | | PFS |
| Particles ≥25 μm (particles/mL) | ≤5 | Results reported from bulk |
| | | PFS |
| Particles ≥50 μm (particles/mL) | ≤2 | Results reported from bulk |
| | | PFS |
| Volume in Container | 0.07 mL minimum expellable | Results reported from bulk |
| | volume | PFS |
| Container Closure Integrity | n/a | No leak detected |
| Maximum injection force (N) | ≤28.2 | Results reported from bulk |
| | | PFS |
| Deliverable Volume (mL) | n/a | 0.070 ≤ x < 0.10 |
| Maximum use torque | ≤13 | Results reported from bulk |
| (N · m) | | PFS |
| Dye Leak (Blister) | n/a | No dye detected |

The Identity by Dot Blot test may be performed by an immunoblotting (dot blot) method to ensure material identity, and identify of the drug product may be confirmed by immunoassay. The Total Protein Content method may be conducted to ensure process consistency in formulating to the target concentration. As indicated in the table above, formulation development robustness studies demonstrated that the formulation is robust against a ±10% variation in protein concentration. The capillary electrophoresis sodium dodecyl sulfate (CE-SDS) method may be conducted under reducing conditions to monitor truncated forms of aflibercept and assure a minimum purity of intact aflibercept. Purity, R1, and LMW (low molecular weight) less R1 may be tested at release for bulk prefilled syringe drug products to ensure material is produced consistently with low levels of R1 and LMW less R1. LMW formation is a stability-indicating attribute and increased levels have the potential to impact potency; therefore, this test may also be performed on stability for bulk prefilled syringes and prefilled syringe drug products to ensure R1 and LMW less R1 levels do not exceed the end-of-shelf-life.

The CE-SDS method may be conducted under non-reducing conditions to assure disulfide bond integrity and a minimum purity of intact aflibercept. Purity may be tested to ensure the material is produced consistently with high purity levels and maintains a high purity level through the end-of-shelf-life. Size-exclusion ultra-performance liquid chromatography (SE-UPLC) may be used to control size variants (aggregated forms) under native conditions. Purity and HMW may be tested at bulk prefilled syringe drug products release to ensure material is produced consistently with low levels of HMW. HMW formation is a stability-indicating attribute and increased levels have the potential to impact potency and immunogenicity. Thus, this test may be performed on stability for bulk prefilled syringes, and prefilled syringe drug products to ensure HMW levels do not exceed the end-of-shelf-life criteria.

Imaged capillary isoelectric focusing (iCIEF) may be used to control charge distribution. In addition, the test article electropherogram must be qualitatively similar to the reference standard electropherogram provided in the procedure. Polysorbate 20 (PS20) may be added to the formulation at low levels to stabilize the protein at air-liquid or solid-liquid interfaces. Such destabilization could occur due to agitation stress during product handling during different stages of manufacturing, filling, shipping, and use, and is typically manifested as an increase in the level of HMW protein species in the product. Isoaspartate is measured using an ISOQUANT® assay to estimate the level of degradation due to isomerization and deamidation of aspartate and asparagine residues in aflibercept samples. The endotoxin release acceptance criteria of ≤0.2 EU/mL are set based on regulatory safety criteria for intraocular fluids. The acceptance criteria are supported by the DP manufacturing experience with endotoxin levels remaining <0.05 EU/mL for all DP batches.

Sterility is tested at bulk prefilled syringe drug product release to ensure microbial control of the final product. Container closure integrity testing is performed in lieu of sterility as part of the stability program. The device labeling and assembly activities are performed using validated automatic equipment, which reduces the process variability associated with manual assembly. Assembly steps with the potential to impact sterility and container closure integrity of the filled syringes are 100% monitored for force-displacement requirements as specified in the equipment User Requirement Specification. Assembled syringes that do not meet the force-displacement requirements are automatically rejected by the equipment. Additionally, during the PPQ, it was demonstrated that the sterility of the drug product and container closure integrity were maintained throughout the automatic manufacturing process. The acceptance criteria for sterility may be set in accordance with USP <71>, Ph. Eur. 2.6.1, and/or JP <4.06>. A microscopic method is used to measure the subvisible particulate matter content in the bulk prefilled syringe drug product according to USP <787> Subvisible Particulate Matter in Therapeutic Protein Injections, USP <788> Particulate Matter in Injections, USP <789> Particulate Matter In Ophthalmic Solutions, Ph. Eur. 2.9.19. Particulate contamination—sub-visible particulates, and JP<6.07> Insoluble Particulate Matter Test for Injections.

The sterility method may use a mass extraction technique to perform a nondestructive, quantitative measurement to detect leakage (USP <1207> Package Integrity Evaluation—Sterile Products). Sterility testing may be performed at release in lieu of container closure integrity for the bulk prefilled syringe drug product.

Maximum injection force may be measured at bulk prefilled syringe drug product end-of-shelf-life and prefilled syringe drug product release and end-of-shelf-life. An instrument may be used to measure the maximum injection force required to push a plunger stopper to expel the liquid out of the syringe. The criteria are set to ensure that the maximum force required to expel the liquid out of the syringe does not exceed the capability of the 5th percentile female pressing with an index finger. This method is adapted from International Organization Standard (ISO) ISO11040-4 and ISO 11040-8.

Devices of the present disclosure are operable to provide accurate measurements in delivering large volumes of vitreous with high precision by minimizing instances of user error in improperly setting a dose line. As described in detail above, the various designs and configurations of the one or more components of the devices described herein (e.g., a plunger rod, a flange piece, etc.) may provide dosage precision by controlling a priming distance and a dosage delivery distance of the device, thereby removing user determination in setting the device at each respective configuration.

Features enumerated above have been described within the context of particular embodiments. However, as one of ordinary skill in the art would understand, features and aspects of each embodiment may be combined, added to other embodiments, subtracted from an embodiment, etc. in any manner suitable to assist with controlled preparation and/or delivery of a drug.

Aspects of the embodiments disclosed herein are described with respect to priming drug delivery devices and removing excess air bubbles from within drug delivery devices, and some embodiments disclosed herein are described as being particular types of drug delivery devices (e.g., pre-filled syringes). Aspects of the present disclosure may also be employed and/or found in other types of drug delivery devices (e.g., fillable syringes, pipettes, and the like). For example, devices having features according to the present disclosure may provide more precise means for transferring a volume of a drug substance or other fluid from one container to another, such as from a vial to a syringe. The precision in fluid transfer afforded by embodiments disclosed herein may reduce or minimize unwanted overfilling and/or decrease wastage of a drug substance.

While a number of embodiments are presented herein, multiple variations on such embodiments, and combinations of elements from one or more embodiments, are possible and are contemplated to be within the scope of the present disclosure. Moreover, those skilled in the art will appreciate that the conception upon which this disclosure is based may readily be used as a basis for designing other devices, methods, and systems for carrying out the several purposes of the present disclosure.

Embodiments of the present disclosure may include the following features:

Item 1. A drug delivery device, comprising: a body storing a medicament, wherein the body is silicone-free; a plunger rod having a proximal portion, a distal portion contacting a stopper inside the body, and a pair of protrusions extending outwards from the proximal portion; and a flange coupled to the body, the flange including a proximal collar and a pair of slots positioned along the proximal collar; wherein the flange is configured such that when the pair of protrusions and the pair of slots are misaligned with one another, distal movement of the plunger rod relative to the body is limited upon the pair of protrusions contacting the proximal collar to prime the drug delivery device by expelling air or at least a portion of the medicament stored in the body; and wherein the flange is configured such that when the pair of protrusions and the pair of slots are aligned with one another, distal movement of the plunger rod relative to the body is limited upon the pair of protrusions extending into the pair of slots to deliver the medicament from the body.

Item 2. The drug delivery device of item 1, wherein the plunger rod includes a pair of extensions extending distally from the proximal portion, and the flange includes a channel defined by an interior surface and a pair of grooves formed on the interior surface.

Item 3. The drug delivery device of item 2, wherein the flange is configured such that the pair of extensions are misaligned with the pair of grooves when the pair of protrusions and the pair of slots are misaligned with one another.

Item 4. The drug delivery device of item 2, wherein the interior surface is configured to engage the pair of extensions when the pair of protrusions and the pair of slots are misaligned and the plunger rod translates distally through the channel via a first stroke; and wherein the pair of grooves is configured to receive the pair of extensions when the pair of protrusions and the pair of slots are aligned and the plunger rod translates distally through the channel via a second stroke.

Item 5. The drug delivery device of item 4, wherein the flange is configured to compress the pair of extensions radially inwards relative to the proximal portion of the plunger rod upon the interior surface engaging the pair of extensions as the plunger rod translates distally relative to the flange by the first stroke while the pair of protrusions and the pair of slots are misaligned.

Item 6. The drug delivery device of item 4, wherein the flange is configured to receive the pair of extensions in the pair of grooves upon the plunger rod rotating relative to the flange to align the pair of protrusions with the pair of slots.

Item 7. The drug delivery device of item 5, wherein the pair of extensions are configured to expand radially outwards relative to the proximal portion upon the pair of grooves receiving the pair of extensions as the plunger rod rotates relative to the flange to align the pair of protrusions with the pair of slots.

Item 8. The drug delivery device of item 5, wherein the pair of extensions are configured to translate distally through the pair of grooves in response to the plunger rod translating distally relative to the flange by the second stroke to position the pair of protrusions into the pair of slots.

Item 9. The drug delivery device of item 2, wherein the flange includes a pair of openings formed in the proximal collar, the pair of openings are configured to at least partially receive the pair of extensions when the plunger rod is coupled to the flange prior to the pair of protrusions contacting the proximal collar to prime the drug delivery device.

Item 10. The drug delivery device of item 9, wherein the flange is configured to inhibit proximal and rotational movement of the plunger rod relative to the body while the pair of extensions are received within the pair of openings; and wherein the pair of extensions are configured to exit the pair of openings upon distal movement of the plunger rod relative to the body to prime the drug delivery device.

Item 11. The drug delivery device of item 1, wherein the body and the stopper are silicone-free such that an interior surface of the body and an exterior surface of the stopper each excludes a coating layer that includes silicone.

Item 12. The drug delivery device of item 1, wherein the distal portion of the plunger rod includes a neck and a stem that is positioned distally of the neck, the plunger rod includes a cross-sectional shape that varies along a longitudinal length of the neck; and
wherein the flange includes an opening that is configured to interface with the neck and the stem to control longitudinal and rotational movement of the plunger rod relative to the flange.

Item 13. The drug delivery device of item 12, wherein the neck includes a first portion having a first cross-sectional shape that is configured to permit distal movement of the plunger rod relative to the flange and inhibit rotational movement of the plunger rod relative to the flange when the first portion is received within the opening.

Item 14. The drug delivery device of item 13, wherein the neck includes a second portion that is positioned distally relative to the first portion, the second portion having a second cross-sectional shape that is configured to permit distal movement and rotational movement of the plunger rod relative to the flange when the second portion is received within the opening.

Item 15. The drug delivery device of item 14, wherein the neck includes a third portion that is positioned distally relative to the second portion, the third portion having a third cross-sectional shape that is configured to permit distal movement of the plunger rod relative to the flange when the third portion is received within the opening and inhibit proximal movement of the plunger rod relative to the flange when the third portion is rotatably misaligned with the opening.

Item 16. A drug delivery device, comprising: a body containing an aqueous pharmaceutical formulation comprising a VEGF antagonist; a plunger rod having a proximal end, a distal end contacting a stopper inside the body, and a pair of protrusions extending outwards from the proximal end; and a flange coupled to the body, the flange including a proximal collar and a pair of slots positioned along the proximal collar; wherein the flange is configured such that when the pair of protrusions and the pair of slots are misaligned with one another, distal movement of the plunger rod relative to the body is limited upon the pair of protrusions contacting the proximal collar to prime the drug delivery device by expelling air or at least a portion of the aqueous pharmaceutical formulation in the body; and wherein the flange is configured such that when the pair of protrusions and the pair of slots are aligned with one another, distal movement of the plunger rod relative to the body is limited upon the pair of protrusions extending into the pair of slots to deliver the VEGF antagonist from the body.

Item 17. The drug delivery device of item 16, wherein the VEGF antagonist includes ranibizumab, bevacizumab, conbercept, pegaptanib, brolucizumab, aflibercept.

Item 18. The drug delivery device of item 17, wherein the VEGF antagonist is aflibercept in a concentration of about 114.3 mg/mL.

Item 19. The drug delivery device of item 16, wherein the VEGF antagonist is aflibercept and the aqueous pharmaceutical formulation further comprises a stabilizing agent and a buffering agent.

Item 20. The drug delivery device of item 19, wherein the stabilizing agent includes one of arginine monohydrochloride, Sucrose, and Polysorbate 20; and wherein the buffering agent includes histidine.

Item 21. The drug delivery device of item 16, wherein the body is configured to store a predetermined volume of the VEGF antagonist stored in a volume from about 150 L to about 250 L.

Item 22. The drug delivery device of item 16, wherein the aqueous pharmaceutical formulation includes: about 100 mg/ml of a VEGF receptor fusion protein; about 10-100 mM L-arginine; sucrose; a histidine-based buffer; and a surfactant.

Item 23. The drug delivery device of item 22, wherein the VEGF receptor fusion protein includes two polypeptides that each comprises an immunoglobin-like (Ig) domain 2 of VEGFR1, an Ig domain 3 of VEGFR2, and a multimerizing component.

Item 24. The drug delivery device of item 23, wherein the VEGF receptor fusion protein has less than about 3.5% high molecular weight species.

Item 25. The drug delivery device of item 22, wherein the aqueous pharmaceutical formulation has a pH of about 5.0 to about 6.8.

Item 26. The drug delivery device of item 16, wherein the aqueous pharmaceutical formulation comprises a VEGF receptor fusion protein having a mass ranging between about 2 mg to about 8 mg.

Item 27. The drug delivery device of item 26, wherein the VEGF receptor fusion protein includes aflibercept.

Item 28. The drug delivery device of item 16, wherein the aqueous pharmaceutical formulation comprises about 114.3 mg/ml of a VEGF receptor fusion protein as the VEGF antagonist, about 50 mM arginine monohydrochloride as the stabilizing agent, and about 10 mM histidine as the buffering agent.

Item 29. The drug delivery device of item 16, wherein the aqueous pharmaceutical formulation comprises: about 40 mg/ml VEGF receptor fusion protein; about 10 mM sodium phosphate; about 40 mM NaCl; about 0.03% polysorbate 20; about 5% sucrose; and a pH of about 6.2.

Item 30. The drug delivery device of item 16, wherein the VEGF antagonist is prefilled within the body during an assembly of the body, the stopper, the plunger rod, and the flange of the drug delivery device such that the drug delivery device is a pre-filled syringe.

Item 31. A packaging for housing a drug delivery device, comprising: a flexible tray and a removable cover adhered to the flexible tray, wherein the flexible tray includes: an opening; a cavity including a plurality of cavity portions; a plurality of sidewalls at least partially defining the cavity; a base at least partially defining the cavity; and a lip surrounding the opening and extending radially outwards from the cavity to define a periphery of the flexible tray, wherein the removable cover is adhered to the lip; wherein the flexible tray is configured to house the drug delivery device in the cavity with at least a portion of the drug delivery device contained within each of the plurality of cavity portions; and wherein the drug delivery device is a pre-filled syringe including an aqueous pharmaceutical formulation comprising a single dose of about 8 mg or more of a VEGF antagonist.

Item 32. The packaging of item 31, wherein the plurality of cavity portions are connected to one another by narrowed portions, and each of the narrowed portions includes a first sidewall, a second sidewall, and a base that are collectively configured to receive at least a portion of a body of the drug delivery device.

Item 33. The packaging of item 32, wherein one of the first sidewall, the second sidewall, or the base of each of the narrowed portions includes a geometric feature extending away from the first sidewall, the second sidewall, and the base and projecting into the narrowed portion.

Item 34. The packaging of item 33, wherein the geometric feature is configured to abut against the portion of the body of the drug delivery device that is contained within the narrowed portion to position the body away from the first sidewall, the second sidewall, and the base of the narrowed portion.

Item 35. The packaging of item 34, wherein the geometric feature is configured to suspend the body of the drug delivery device within the cavity, thereby forming a gap between an exterior of the body and the plurality of sidewalls and the base of the flexible tray.

Item 36. The packaging of item 31, wherein the drug delivery device includes a body, a plunger rod, and a flange that are securely disposed inside the plurality of cavity portions and sealed inside the flexible tray by the removable cover closing the opening; and wherein the packaging and the drug delivery device are sterilized.

Item 37. The packaging of item 31, wherein the removable cover is permeable to a gaseous sterilant that is configured to sterilize the flexible tray and the drug delivery device disposed inside the cavity during a sterilization of the packaging.

Item 38. The packaging of item 31, wherein the flexible tray includes a projection positioned along the base and aligned with one of the plurality of cavity portions, wherein the projection extends outwards from the flexible tray in a direction opposite from the cavity.

Item 39. The packaging of item 38, wherein the projection is hollow and open towards the cavity of the flexible tray.

Item 40. The packaging of item 38, wherein the projection is configurable to be at least partially deformable in response to an application of force on the projection.

Item 41. The packaging of item 38, wherein the projection is configured to flex the flexible tray in response to an application of force on the projection, thereby at least partially deforming one or more of the plurality of cavity portions and partially decoupling the drug delivery device from the cavity.

Item 42. The packaging of item 38, wherein the projection is dome-shaped and a height of the projection from an apex of the projection to a base of the projection is greater than a distance between the base of the projection and the drug delivery device contained within the cavity and overlying the projection.

Item 43. The packaging of item 38, wherein the projection includes a bulbous shape, and a diameter of the projection defines a widest portion of the bulbous shape where the projection meets the base of the flexible tray.

Item 44. The packaging of item 38, wherein the projection includes a dome having a widest diameter at the base of the flexible tray, wherein the projection extends directly outwards from the base of the flexible tray such that the widest diameter of the dome is connected at the base.

Item 45. The packaging of item 38, wherein the VEGF antagonist that is pre-filled in the drug delivery device includes aflibercept with a concentration of about 114.3 mg/mL.

Item 46. A method of assembling a drug delivery device, comprising: filling a body of the drug delivery device with a substance; inserting a stopper of the drug delivery device into the body to fluidly seal the substance inside the body; coupling the body with a finger flange of the drug delivery device by inserting a proximal flange of the body into a lateral slot of the finger flange; inserting a plunger rod of the drug delivery device through the finger flange and into the body to an extent such that the plunger rod contacts the stopper; and coupling the plunger rod to the finger flange by inserting an extension of the plunger rod into a lateral opening of the finger flange.

Item 47. The method of item 46, wherein filling the body with the substance comprises: filling the body to a maximum target fill volume, wherein the maximum target fill volume includes a single dose having a predetermined volume that is less than the maximum target fill volume.

Item 48. The method of item 47, wherein inserting the stopper into the body comprises: forming a space within the body between the stopper and the substance as the stopper is inserted into the body, wherein the space defines an air pocket.

Item 49. The method of item 48, wherein the space includes a headspace volume such that the maximum target fill volume of the substance and the headspace volume of the air pocket are collectively disposed between a distal end of the body and the stopper.

Item 50. The method of item 49, wherein the substance includes a VEGF antagonist, the maximum target fill volume is between about 180 μL and about 250 μL, the predetermined volume of the single dose delivered from the drug delivery device is about 90 L or less, and the body and the stopper are silicone-free.

Item 51. The method of item 46, wherein inserting the stopper into the body comprises: positioning the stopper near a proximal portion of the body that is adjacent to the proximal flange; and generating a vacuum pressure within the body to reposition the stopper from the proximal portion of the body towards a distal portion of the body that contains the substance, thereby sealing the substance in the body with the stopper.

Item 52. The method of item 51, wherein the vacuum pressure generated within the body is to an extent such that the stopper is repositioned to a predetermined location within the distal portion of the body to maintain a maximum target fill volume of the substance that is pre-filled within the body.

Item 53. The method of item 46, wherein coupling the body and the finger flange comprises: engaging an upper surface of the proximal flange with a pair of ribs of the finger flange and a lower surface of the proximal flange with a lip of the finger flange as the proximal flange is inserted into the lateral slot.

Item 54. The method of item 53, wherein the pair of ribs are at least partially deflectable in response to engaging the proximal flange, the pair of ribs being positioned inside the lateral slot of the finger flange.

Item 55. The method of item 46, wherein coupling the body and the finger flange comprises: engaging a side surface of the body with a pair of movable tabs of the finger flange as the proximal flange is inserted into the lateral slot.

Item 56. The method of item 55, wherein the pair of movable tabs are at least partially deflectable in response to engaging the body, the pair of movable tabs being positioned outside the lateral slot of the finger flange.

Item 57. The method of item 46, wherein inserting the plunger rod through the finger flange and into the body comprises: applying an insertion force of about 15 N or less to the plunger rod for positioning a distal end of the plunger rod within the body adjacent to the stopper.

Item 58. The method of item 57, wherein inserting the plunger rod through the finger flange and into the body comprises: abutting a distal end of the plunger rod against a proximal end of the stopper without coupling the plunger rod to the stopper.

Item 59. The method of item 57, wherein inserting the plunger rod through the finger flange and into the body comprises: inserting a distal end of the plunger rod into a proximal end of the stopper without coupling the plunger rod to the stopper.

Item 60. The method of item 46, wherein coupling the plunger rod to the finger flange comprises: deflecting the extension of the plunger rod laterally inwards upon engaging a proximal edge of the finger flange as the plunger rod is inserted distally through the finger flange; and deflecting the extension laterally outwards upon entering the lateral opening of the finger flange as the plunger rod is inserted further distally through the finger flange.

Item 61. A method of treating or preventing an angiogenic eye disorder, comprising: administering a single initial dose of about 8 mg or more of a VEGF antagonist that is pre-filled in a first drug delivery device to an eye of a patient; administering one or more secondary doses of about 8 mg or more of the VEGF antagonist to the eye of the patient by one or more second drug delivery devices that are pre-filled with the VEGF antagonist; and administering one or more tertiary doses of about 8 mg or more of the VEGF antagonist to the eye of the patient by one or more third drug delivery devices that are pre-filled with the VEGF antagonist; wherein the first drug delivery device, the one or more second drug delivery devices, and the one or more third drug delivery devices each include a body storing the VEGF antagonist, a finger flange coupled to the body, and a plunger rod movably coupled to the finger flange for priming the body prior to administering the single initial dose, the one or more secondary doses, and the one or more tertiary doses.

Item 62. The method of item 61, wherein the plunger rod of each of the first drug delivery device, the one or more second drug delivery devices, and the one or more third drug delivery devices includes a protrusion extending outwards from a proximal end of the plunger rod; and wherein the finger flange of each of the first drug delivery device, the one or more second drug delivery devices, and the one or more third drug delivery devices includes a proximal collar and a slot extending distally from the proximal collar.

Item 63. The method of item 62, wherein the protrusion is configured to abut against the proximal collar upon the plunger rod moving distally relative to the finger flange to prime the body; and wherein the protrusion is configured to abut against the slot upon the plunger rod moving distally relative to the finger flange to administer the single initial dose, the one or more secondary doses, and the one or more tertiary doses.

Item 64. The method of item 61, wherein the body of each of the first drug delivery device, the one or more second drug delivery devices, and the one or more third drug delivery devices is silicone-free.

Item 65. The method of item 64, wherein the body of each of the first drug delivery device, the one or more second drug delivery devices, and the one or more third drug delivery devices is a syringe having a maximum volume of 0.5 mL or 1.0 mL.

Item 66. The method of item 61, wherein the body of each of the first drug delivery device, the one or more second drug delivery devices, and the one or more third drug delivery devices includes a stopper that is silicone-free.

Item 67. The method of item 61, wherein administering the one or more secondary doses comprises: administering each of the one or more secondary doses about 2 weeks to about 4 weeks after an immediately preceding dose from the one or more secondary doses.

Item 68. The method of item 67, wherein a time interval for administering each of the one or more secondary doses is based on an indication of the angiogenic eye disorder.

Item 69. The method of item 61, wherein administering the one or more tertiary doses comprises: administering each of the one or more tertiary doses about 4 weeks, about 8 weeks, or about 12 weeks after an immediately preceding dose from the one or more tertiary doses.

Item 70. The method of item 61, wherein the VEGF antagonist includes aflibercept and the single initial dose, the one or more secondary doses, and the one or more tertiary doses have a volume of about 100 μL or less.

Item 71. The method of item 70, wherein the first drug delivery device, the one or more second drug delivery devices, and the one or more third drug delivery devices are each configured to treat or prevent the angiogenic eye disorder by administering doses of aflibercept at an interval and a quantity whereby a clearance of free aflibercept from an ocular compartment in the eye is about 0.3 mL/day to about 0.46 mL/day after intravitreal injection of aflibercept to the eye.

Item 72. The method of item 71, wherein a time for the clearance for free aflibercept to reach a lower limit of quantitation (LLOQ) in the ocular compartment of the eye after said intravitreal injection of aflibercept is about 15 weeks.

Item 73. The method of item 71, wherein a time for free aflibercept to reach a lower limit of quantitation (LLOQ) in a plasma of the eye after said intravitreal injection of aflibercept is about 3.5 weeks to about 3.8 weeks.

Item 74. The method of item 61, further comprising: administering one or more non-scheduled doses of the VEGF antagonist to the eye of the patient by one or more additional drug delivery devices that are pre-filled with the VEGF antagonist on a pro re nata basis in response to anatomic findings from the eye of the patient; wherein the one or more non-scheduled doses are capped at a minimum dosing frequency of one at least every 2 months to 4 months.

Item 75. The method of item 61, wherein the angiogenic eye disorder includes one of age-related macular degeneration (AMD), macular edema (ME), retinal vein occlusion (RVO), diabetic macular edema (DME), neovascularization, neovascular glaucoma, post-surgical fibrosis in glaucoma, proliferative vitreoretinopathy (PVR), pannus, pterygium, vascular retinopathy, and diabetic retinopathy.

1. A drug delivery device, comprising:
a body storing a medicament, wherein the body is silicone-free;
a plunger rod having a proximal end, a distal end contacting a stopper inside the body, and a pair of protrusions extending outwards from the proximal end; and
a flange coupled to the body, the flange including a proximal collar and a pair of slots positioned along the proximal collar;
wherein the flange is configured such that when the pair of protrusions and the pair of slots are misaligned with one another, distal movement of the plunger rod relative to the body is limited upon the pair of protrusions contacting the proximal collar to prime the drug delivery device by expelling air or at least a portion of the medicament stored in the body; and
wherein the flange is configured such that when the pair of protrusions and the pair of slots are aligned with one another, distal movement of the plunger rod relative to the body is limited upon the pair of protrusions extending into the pair of slots to deliver the medicament from the body.

2. The drug delivery device of claim 1, wherein the plunger rod includes a pair of extensions extending distally from the proximal end, and the flange includes a channel defined by an interior surface and a pair of grooves formed on the interior surface.

3. The drug delivery device of claim 2, wherein the flange is configured such that the pair of extensions are misaligned with the pair of grooves when the pair of protrusions and the pair of slots are misaligned with one another.

4. The drug delivery device of claim 2, wherein the interior surface is configured to engage the pair of extensions when the pair of protrusions and the pair of slots are misaligned and the plunger rod translates distally through the channel via a first stroke; and
wherein the pair of grooves is configured to receive the pair of extensions when the pair of protrusions and the pair of slots are aligned and the plunger rod translates distally through the channel via a second stroke.

5. The drug delivery device of claim 4, wherein the flange is configured to compress the pair of extensions radially inwards relative to the proximal end of the plunger rod upon the interior surface engaging the pair of extensions as the plunger rod translates distally relative to the flange by the first stroke while the pair of protrusions and the pair of slots are misaligned.

6. The drug delivery device of claim 4, wherein the flange is configured to receive the pair of extensions in the pair of grooves upon the plunger rod rotating relative to the flange to align the pair of protrusions with the pair of slots.

7. The drug delivery device of claim 5, wherein the pair of extensions are configured to expand radially outwards relative to the proximal end upon the pair of grooves receiving the pair of extensions as the plunger rod rotates relative to the flange to align the pair of protrusions with the pair of slots.

8. The drug delivery device of claim 5, wherein the pair of extensions are configured to translate distally through the pair of grooves in response to the plunger rod translating distally relative to the flange by the second stroke to position the pair of protrusions into the pair of slots.

9. The drug delivery device of claim 2, wherein the flange includes a pair of openings formed in the proximal collar, the pair of openings are configured to at least partially receive the pair of extensions when the plunger rod is coupled to the flange prior to the pair of protrusions contacting the proximal collar to prime the drug delivery device.

10. The drug delivery device of claim 9, wherein the flange is configured to inhibit proximal and rotational movement of the plunger rod relative to the body while the pair of extensions are received within the pair of openings; and
wherein the pair of extensions are configured to exit the pair of openings upon distal movement of the plunger rod relative to the body to prime the drug delivery device.

11. The drug delivery device of claim 1, wherein the body and the stopper are silicone-free such that an interior surface of the body and an exterior surface of the stopper each excludes a coating layer that includes silicone.

12. The drug delivery device of claim 1, wherein the distal portion of the plunger rod includes a neck and a stem that is positioned distally of the neck, the plunger rod includes a cross-sectional shape that varies along a longitudinal length of the neck; and
wherein the flange includes an opening that is configured to interface with the neck and the stem to control longitudinal and rotational movement of the plunger rod relative to the flange.

13. The drug delivery device of claim 12, wherein the neck includes a first portion having a first cross-sectional shape that is configured to permit distal movement of the plunger rod relative to the flange and inhibit rotational movement of the plunger rod relative to the flange when the first portion is received within the opening.

14. The drug delivery device of claim 13, wherein the neck includes a second portion that is positioned distally relative to the first portion, the second portion having a second cross-sectional shape that is configured to permit distal movement and rotational movement of the plunger rod relative to the flange when the second portion is received within the opening.

15. The drug delivery device of claim 14, wherein the neck includes a third portion that is positioned distally relative to the second portion, the third portion having a third cross-sectional shape that is configured to permit distal movement of the plunger rod relative to the flange when the third portion is received within the opening and inhibit proximal movement of the plunger rod relative to the flange when the third portion is rotatably misaligned with the opening.

16.-75. (canceled)
